(19) United States
(12) Patent Application Publication
(10) Pub. No.: US 2026/0107682 A1
(43) Pub. Date: Apr. 16, 2026

(54) COMPOUND, ORGANIC ELECTROLUMINESCENT DEVICE, AND DISPLAY APPARATUS

(71) Applicant: BOE TECHNOLOGY GROUP CO., LTD., Beijing (CN)

(72) Inventors: Rongrong GAO, Beijing (CN); Wenzhu JING, Beijing (CN); Lei CHEN, Beijing (CN); Tong WU, Beijing (CN)

(73) Assignee: BOE TECHNOLOGY GROUP CO., LTD., Beijing (CN)

(21) Appl. No.: 19/281,797

(22) Filed: Jul. 28, 2025

(30) Foreign Application Priority Data
Oct. 14, 2024 (CN) 202411435021.6

(51) Int. Cl. H10K 85/60; C07B 59/00; C07C 13/64; C07D 215/02; C07D 217/02; C07D 307/91; C07D 307/92; C07D 333/76; C07D 493/04; C07D 495/04; C09K 11/02; H10K 50/11

(52) U.S. Cl. CPC H10K 85/624; C07B 59/001; C07B 59/002; C07C 13/64; C07D 215/02; C07D 217/02; C07D 307/91; C07D 307/92; C07D 333/76; C07D 493/04; C07D 495/04; C09K 11/02; H10K 85/626; H10K 85/6574; C07B 2200/05; H10K 50/11

(57) ABSTRACT

The disclosure provides a compound, an organic electroluminescent device, and a display apparatus, relating to the field of display technology. The structure of the compound is represented by Chemical Formula 1. When applied to organic electroluminescent devices, this compound can enhance device performance.

### CROSS-REFERENCE TO RELATED APPLICATION

The present disclosure claims priority to Chinese Patent Application No. 202411435021.6, entitled “Compound, Organic Electroluminescent Devices, and Display Apparatus”, filed on Oct. 14, 2024. The entire content of the aforementioned Chinese patent application is incorporated herein by reference.

### TECHNICAL FIELD

The present disclosure relates to the field of display technology, specifically to a compound, an organic electroluminescent device, and a display apparatus.

### BACKGROUND

OLED (Organic Light-Emitting Device) is an electroluminescent device that has gained widespread application in solid-state lighting and display fields due to its advantages of self-emission, high resolution, low power consumption, high color saturation, wide color gamut, fast screen response time, and flexibility. The rapid adoption of OLEDs, substantial market demand, and the fast iteration of electronic products have driven research and development in functional materials, including hole injection materials, hole transport materials, hole blocking materials, light-emitting host materials, light-emitting dopants, electron blocking materials, electron transport materials, and electron injection materials. These efforts aim to improve OLED) performances such as luminous efficiency, driving voltage, lifespan, and color purity. Developing more compounds suitable for OLEDs is of great significance for advancing the further development and application of OLED technology.

It should be noted that the information disclosed in the Background section above is only for enhancing the understanding of the background of the present disclosure, and thus may include information that does not constitute prior art known to those of ordinary skill in the art.

### SUMMARY

The present disclosure provides a compound, an organic electroluminescent device, and a display apparatus, which improve the performance of the organic electroluminescent device.

According to one aspect of the present disclosure, a compound is provided, having a structural formula represented by Chemical Formula 1:

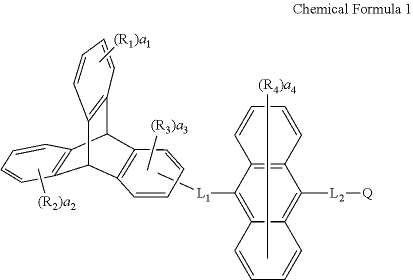

wherein, L₁ and L₂ are each independently selected from a single bond or a linking group, the linking group being selected from divalent arylene or divalent heteroarylene;
Q is a substituted or unsubstituted 2 to 5 ring-fused aryl group, or a substituted or unsubstituted 2 to 5 ring-fused heteroaryl group;
R₁ to R₃ are the same or different from each other and are each independently selected from a group consisting of deuterium, fluorine, chlorine, substituted or unsubstituted alkyl having 1 to 12 carbon atoms, substituted or unsubstituted haloalkyl having 1 to 12 carbon atoms, substituted or unsubstituted alkoxy having 1 to 12 carbon atoms, substituted or unsubstituted cycloalkyl having 3 to 10 carbon atoms, substituted or unsubstituted aryl having 6 to 60 carbon atoms, and substituted or unsubstituted heteroaryl having 3 to 60 carbon atoms;
R4 is selected from a group consisting of deuterium, halogen, cyano, substituted or unsubstituted alkylamino having 1 to 20 carbon atoms, substituted or unsubstituted alkyl having 1 to 20 carbon atoms, substituted or unsubstituted alkoxy having 1 to 20 carbon atoms, substituted or unsubstituted aryloxy having 6 to 30 ring carbon atoms, substituted or unsubstituted arylthio having 6 to 30 ring carbon atoms, substituted or unsubstituted aryl having 6 to 30 ring carbon atoms, and substituted or unsubstituted heterocyclic group having 5 to 30 ring carbon atoms;
a₁ is an integer from 0 to 4; when a₁ is greater than 1, any two R₁ are the same or different;
a₂ is an integer from 0 to 4; when a₂ is greater than 1, any two R₂ are the same or different;
a₂ is an integer from 0 to 3; when a₃ is greater than 1, any two R₃ are the same or different;
a₄ is an integer from 0 to 8; when a₄ is greater than 1, any two R₄ are the same or different.

According to another aspect of the present disclosure, an organic electroluminescent device is provided, including an anode, an organic light-emitting layer, and a cathode sequentially stacked in layers; wherein the organic light-emitting layer includes the aforementioned compound.

According to yet another aspect of the present disclosure, a display apparatus is provided, including the aforementioned organic electroluminescent device.

It is to be understood that the general description above and the detailed description below are exemplary and explanatory only, and do not limit the present disclosure.

### BRIEF DESCRIPTION OF THE DRAWINGS

The drawings here are incorporated into the specification and constitute a part of the specification, show embodiments in consistent with the present disclosure, and are used together with the specification to explain principles of the present disclosure, Obviously, the drawings in the following description are only some embodiments of the present disclosure. For those skilled in the art, other drawings can be obtained based on these drawings without creative work.

FIG. 1 shows the preparation materials and yields of some compounds.

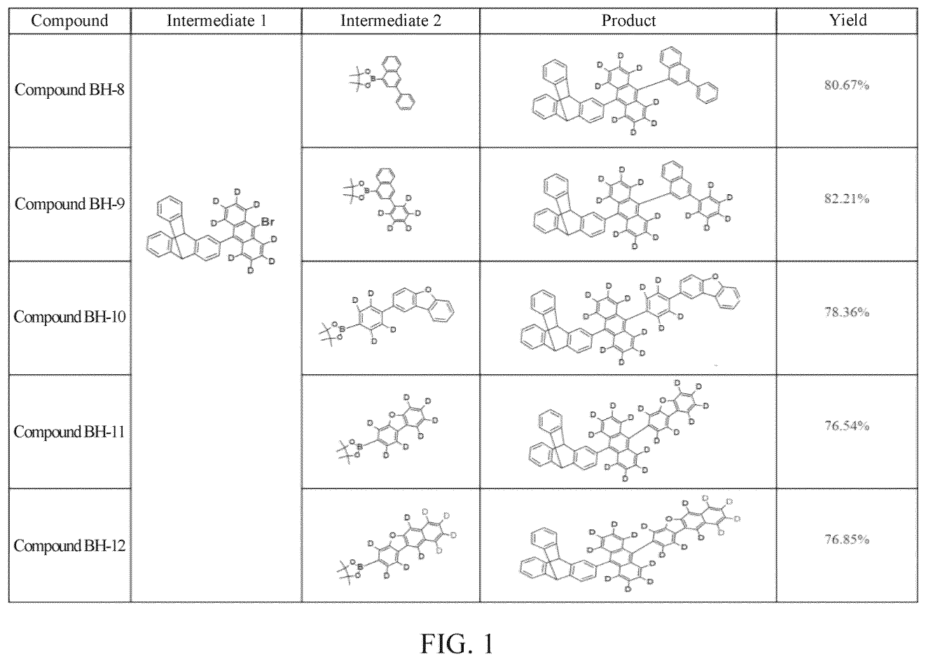

FIG. 2 shows the preparation materials and yields of some compounds.

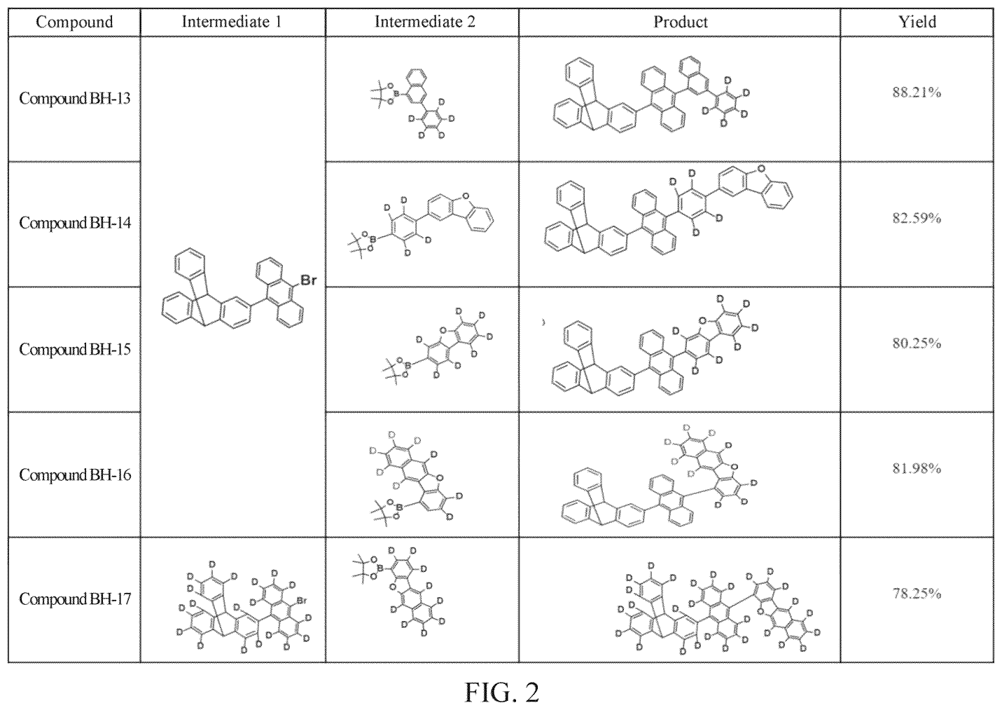

FIG. 3 shows the electron cloud distribution diagrams of some compounds simulated using molecular modeling software.

FIG. 4 shows the performance parameters of some compounds.

FIG. 5 shows the performance parameters of some compounds.

FIG. 6 is a schematic structural diagram of an organic electroluminescent device in one embodiment of the present disclosure.

### DETAILED DESCRIPTION

Example embodiments will now be described more fully with reference to the accompanying drawings. However, the example embodiments can be embodied in a variety of forms and should not be construed as limited to the embodiments set forth herein; rather, these embodiments are provided so that the present disclosure will be thorough and complete and fully convey the concepts of the example embodiments to those skilled in the art. The described features, structures, or characteristics may be incorporated in one or more embodiments in any suitable manner. Like reference numerals refer to the same or similar structures, and detailed description thereof will be omitted. In addition, the drawings are merely schematic illustrations of the present disclosure and are not necessarily drawn to scale.

The terms “a”, “an”, “the”, “said”, and “at least one” are used to indicate that there are one or more elements/components/and the like; the terms “including” and “having” are used to refer to an open-ended inclusion and refer to that there may be additional elements/components/and the like in addition to the listed elements/components/and the like; the terms “first”, “second”, “third”, and the like are used only as labels, not to the number of objects thereof.

In the present disclosure, the descriptive phrase “ . . . each independently selected from” should be interpreted broadly. It may indicate that, among different groups, the specific options represented by identical symbols do not affect each other, or it may mean that, within the same group, the specific options represented by identical symbols do not affect each other. For example, in the statement

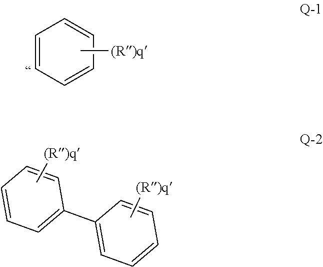

wherein each q′ is independently 0, 1, 2, or 3, and each R″ is independently selected from hydrogen, deuterium, fluorine, or chlorine″, the interpretation is as follows: Formula Q-1 indicates that there are q′ substituents R″ on the benzene ring, where each R″ may be the same or different, and the selection of each R″ is independent of the others; formula Q-2 indicates that each benzene ring in the biphenyl structure has q′ substituents R″, the number of R″ substituents (q′) on the two benzene rings may be same or different, each R″ may be the same or different, and the selection of each R″ is independent of the others.

In the present disclosure, an unpositioned connecting bond refers to a single bond “T” extending from a ring system, which means that one end of the connecting bond can be connected to any position of the ring system through which the bond passes, and the other end can be connected to the rest of the compound molecule.

For example, as shown in Formula (f) below, the naphthyl group represented by Formula (f) is connected to other positions of the molecule through two unpositioned connecting bonds which pass through a dicyclic ring. It means that it can include any possible connection way as shown in Formulas (f-1) to (f-10).

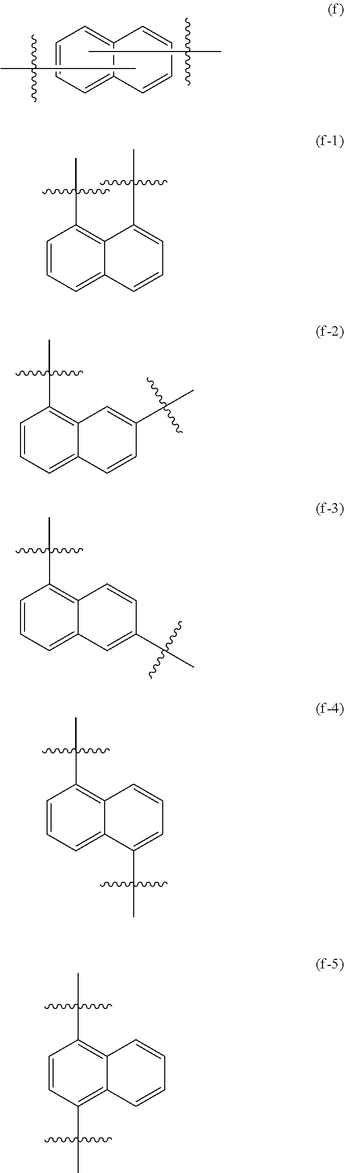

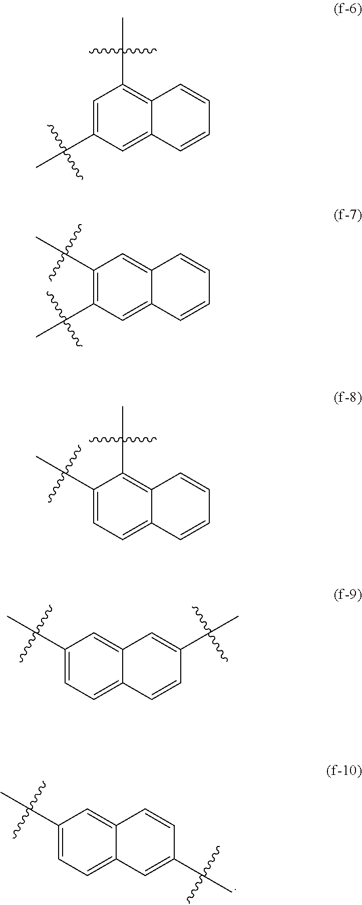 [SMILES: *c1cc(*)c2ccccc2c1 ; *c1cc2ccccc2cc1* ; *c1ccc2ccccc2c1* ; *c1ccc2ccc(*)cc2c1 ; *c1ccc2cc(*)ccc2c1]

For example again, as shown in Formula (X′) below, the phenyl group represented by Formula (X′) is connected to other positions of the molecule through an unpositioned connecting bond extending from one side of the benzene ring. It means that it can include any possible connection way as shown in Formulas (X′-1) to (X′-4).

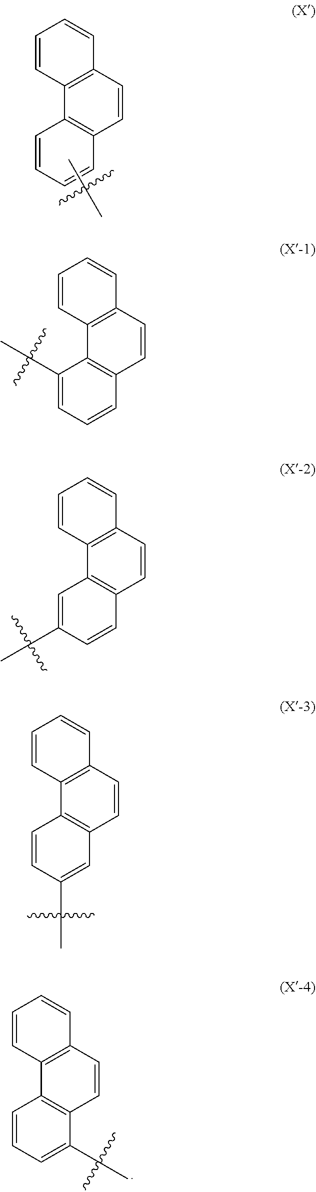

In the embodiment of the disclosure, the unpositioned substituent refers to a substituent connected by a single bond extending from the ring system, which means that the substituent can be connected at any possible position of the ring system. For example, as shown in Formula (Y) below, the substituent R′ group represented by Formula (Y) is connected to the quinoline ring through an unpositioned connecting bond. It means that it can include any possible connection way as shown in Formulas (Y-1) to (Y-7).

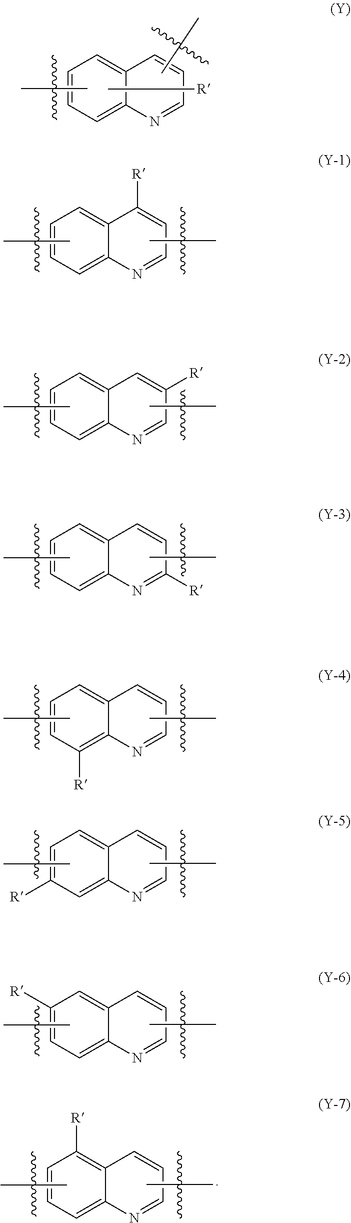

In the present disclosure, for a substituted or unsubstituted group G with M carbon atoms, the number M of carbon atoms of the G group does not count carbon atoms on substituents. For example, the total number of carbon atoms of a methyl-substituted group G having M carbon atoms is M+1. Correspondingly, for a substituted or unsubstituted aryl group or heteroaryl group having M carbon atoms, the number M of carbon atoms does not count carbon atoms on substituents, but counts only carbon atoms on the aromatic ring or the heteroaromatic ring. For instance:

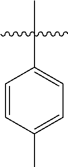 [SMILES: *c1ccc(C)cc1]

is an aryl group having 6 carbon atoms substituted with a methyl group;

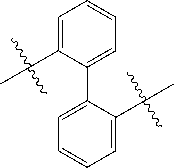 [SMILES: *c1ccccc1-c1ccccc1*]

is an unsubstituted arylene group having 12 carbon atoms.

In the disclosure, unless otherwise specifically defined, the term “hetero” refers to a functional group containing at least one heteroatom selected from B, N, O, S, Se, Si, or P, with the remaining atoms being carbon, hydrogen, and/or deuterium. An unsubstituted alkyl group may be a “saturated alkyl group” without any double or triple bonds.

In the present disclosure, “alkyl” may include a linear alkyl group or a branched alkyl group. The alkyl group may have 1 to 18 carbon atoms. In the present disclosure, numerical ranges such as “1-18” refer to each integer within the given range; for example, “an alkyl group having 1 to 18 carbon atoms” means alkyl groups that may contain 1 carbon atom, 2 carbon atoms, 3 carbon atoms, 4 carbon atoms, 5 carbon atoms, 6 carbon atoms, 7 carbon atoms, 8 carbon atoms, 9 carbon atoms, 10 carbon atoms, 11 carbon atoms, 12 carbon atoms, 13 carbon atoms, 14 carbon atoms, 15 carbon atoms, 16 carbon atoms, 17 carbon atoms, or 18 carbon atoms. In some examples, the alkyl group may also be a small alkyl group having 1 to 5 carbon atoms.

Optionally, the alkyl group is selected from alkyl groups having 1 to 5 carbon atoms. Specific examples include but are not limited to methyl, ethyl, n-propyl, isopropyl, n-butyl, isobutyl, sec-butyl, tert-butyl, and pentyl.

In the present disclosure, “cycloalkyl” refers to a group derived from a saturated cyclic carbon chain structure. The cycloalkyl group may have 5 to 10 carbon atoms. In the present disclosure, numerical ranges such as “5-10” in “a cycloalkyl group having 5 to 10 carbon atoms” refer to each integer within the given range; for example, “a cycloalkyl group having 5 to 10 carbon atoms” means cycloalkyl groups that may contain 5 carbon atoms, 6 carbon atoms, 7 carbon atoms, 8 carbon atoms, 9 carbon atoms, or 10 carbon atoms.

Optionally, specific examples of cycloalkyl groups include but are not limited to cyclopentyl, cyclohexyl, adamantyl, norbornyl, and the like.

In the present disclosure, “aryl” refers to an optional functional group or substituent derived from an aromatic carbon ring. The aryl group may be a monocyclic aryl group (e.g., phenyl) or a polycyclic aryl group. In other words, the aryl group may be a monocyclic aryl group, a fused-ring aryl group, two or more monocyclic aryl groups connected via conjugated carbon-carbon bonds, a monocyclic aryl group and a fused-ring aryl group connected via conjugated carbon-carbon bonds, or two or more fused-ring aryl groups connected via conjugated carbon-carbon bonds. That is, unless otherwise specified, two or more aromatic groups connected via conjugated carbon-carbon bonds may also be regarded as aryl groups in the present disclosure. Among these, fused-ring aryl groups may include bicyclic fused aryl groups (e.g., naphthyl), tricyclic fused aryl groups (e.g., phenanthrenyl, fluorenyl, anthracenyl), and the like. The aryl group does not contain heteroatoms such as B, N, O, S, P, Se, or Si. For example, in the present disclosure, biphenyl, terphenyl, and the like are considered aryl groups. Examples of aryl groups may include but are not limited to phenyl, naphthyl, fluorenyl, anthracenyl, phenanthrenyl, biphenyl, terphenyl, quaterphenyl, pentaphenyl, benzo[9,10]phenanthrenyl, pyrenyl, benzofluoranthenyl, chrysyl, and the like. In the present disclosure, “biphenyl” may be understood as a phenyl-substituted aryl group or as an unsubstituted aryl group. In the present disclosure, the term “arylene” refers to a divalent group formed by an aryl group losing one additional hydrogen atom.

In the present disclosure, a substituted aryl group may be an aryl group in which one or two or more hydrogen atoms are substituted with groups such as deuterium atoms, aryl groups, heteroaryl groups, alkyl groups, cycloalkyl groups, alkoxy groups, or other substituents.

In the present disclosure, “heteroaryl” refers to a monovalent aromatic ring or its derivative containing at least one heteroatom in the ring, where the heteroatom may be at least one selected from B, O, N, P, Si, Se, and S. The heteroaryl group may be a monocyclic heteroaryl group or a polycyclic heteroaryl group. In other words, the heteroaryl group may be a single aromatic ring system or multiple aromatic ring systems connected via conjugated carbon-carbon bonds, where each aromatic ring system is an aromatic monocycle or an aromatic fused ring. Examples of heteroaryl groups may include thienyl, furyl, pyrrolyl, imidazolyl, thiazolyl, oxazolyl, oxadiazolyl, triazolyl, pyridyl, bipyridyl, pyrimidinyl, triazinyl, acridinyl, pyridazinyl, pyrazinyl, quinolinyl, quinazolinyl, quinoxalinyl, phenoxazinyl, phthalazinyl, pyridopyrimidinyl, pyridopyrazinyl, pyrazinopyrazinyl, isoquinolinyl, indolyl, carbazolyl, benzoxazolyl, benzimidazolyl, benzothiazolyl, benzocarbazolyl, benzothienyl, dibenzothienyl, thienothienyl, benzofuryl, phenanthrolinyl, isoxazolyl, thiadiazolyl, benzothiazolyl, phenothiazinyl, silafluorenyl, dibenzofuryl, N-aryl carbazolyl (e.g., N-phenyl carbazolyl), N-heteroaryl carbazolyl (e.g., N-pyridyl carbazolyl), N-alkyl carbazolyl (e.g., N-methyl carbazolyl), and the like, without limitation. Among these, thienyl, furyl, phenanthrolinyl, and the like are heteroaryl groups of the single aromatic ring system type, while N-aryl carbazolyl (e.g., N-phenyl carbazolyl) and N-heteroaryl carbazolyl are heteroaryl groups of the polycyclic system type connected via conjugated carbon-carbon bonds.

In the present disclosure, a substituted heteroaryl group may be a heteroaryl group in which one or two or more hydrogen atoms are substituted with groups such as deuterium atoms, aryl groups, heteroaryl groups, alkyl groups, cycloalkyl groups, alkoxy groups, and the like.

The present disclosure provides a compound represented by Chemical Formula 1:

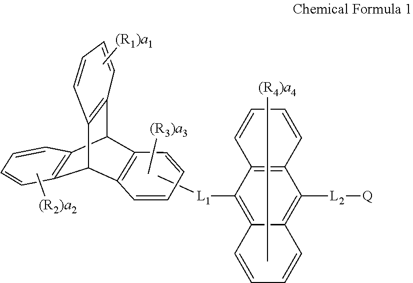

wherein, L₁ and L₂ are each independently selected from a single bond or a linking group, the linking group being selected from divalent arylene or divalent heteroarylene;
Q is a substituted or unsubstituted 2 to 5 ring-fused aryl group, or a substituted or unsubstituted 2 to 5 ring-fused heteroaryl group;
R₁ to R₃ are the same or different from each other and are each independently selected from a group consisting of deuterium, fluorine, chlorine, substituted or unsubstituted alkyl having 1 to 12 carbon atoms, substituted or unsubstituted haloalkyl having 1 to 12 carbon atoms, substituted or unsubstituted alkoxy having 1 to 12 carbon atoms, substituted or unsubstituted cycloalkyl having 3 to 10 carbon atoms, substituted or unsubstituted aryl having 6 to 60 carbon atoms, and substituted or unsubstituted heteroaryl having 3 to 60 carbon atoms;
R₄ is selected from deuterium, halogen, cyano, substituted or unsubstituted alkylamino having 1 to 20 carbon atoms, substituted or unsubstituted alkyl having 1 to 20 carbon atoms, substituted or unsubstituted alkoxy having 1 to 20 carbon atoms, substituted or unsubstituted aryloxy having 6 to 30 ring carbon atoms, substituted or unsubstituted arylthio having 6 to 30 ring carbon atoms, substituted or unsubstituted aryl having 6 to 30 ring carbon atoms, and substituted or unsubstituted heterocyclic group having 5 to 30 ring carbon atoms;
a₁ is an integer from 0 to 4; when a₁ is greater than 1, any two R₁ are the same or different;
a₂ is an integer from 0 to 4; when a₂ is greater than 1, any two R₂ are the same or different;
a₃ is an integer from 0 to 3; when a₃ is greater than 1, any two R₃ are the same or different;
a₄ is an integer from 0 to 8; when a₄ is greater than 1, any two R₄ are the same or different.

The compound provided in the present disclosure is an anthracene-based compound. The anthracene core inherently possesses a high fluorescence quantum yield, enabling blue light emission. However, the anthracene core consists of three benzene rings fused into a planar structure, which is prone to π-π stacking, leading to crystallization of the material. This crystallization can cause clogging of the crucible orifice during the evaporation process. The compound of Chemical Formula 1 according to the present disclosure introduces a triptycene group in addition to the anthracene core. The triptycene group features a three-dimensional paddle-like structure with a large steric volume. The incorporation of this bulky triptycene group modulates the intermolecular forces of the compound, reducing molecular stacking effects and crystallization tendency, thereby improving device lifetime. Moreover, the introduction of the triptycene group reduces the crystallization tendency of the compound, thereby mitigating or eliminating crucible clogging during the evaporation process caused by crystallization, and enhancing the manufacturability of the compound.

Additionally, the bulky triptycene group reduces intermolecular interactions, preventing spectral red-shift of the compound in thin-film states. This avoids efficiency degradation of the organic electroluminescent device due to spectral red-shift. For example, this compound can serve as a blue light host material. Generally, if a blue light host material undergoes spectral red-shift in thin-film states, excitons formed on the host material cannot be efficiently transferred to the blue light dopant material, leading to reduced luminescence efficiency of the dopant and ultimately decreasing the efficiency of the organic electroluminescent device.

In one embodiment of the present disclosure, Q is selected from a group consisting of the groups represented by Chemical Formulas 3-1 to 3-13:

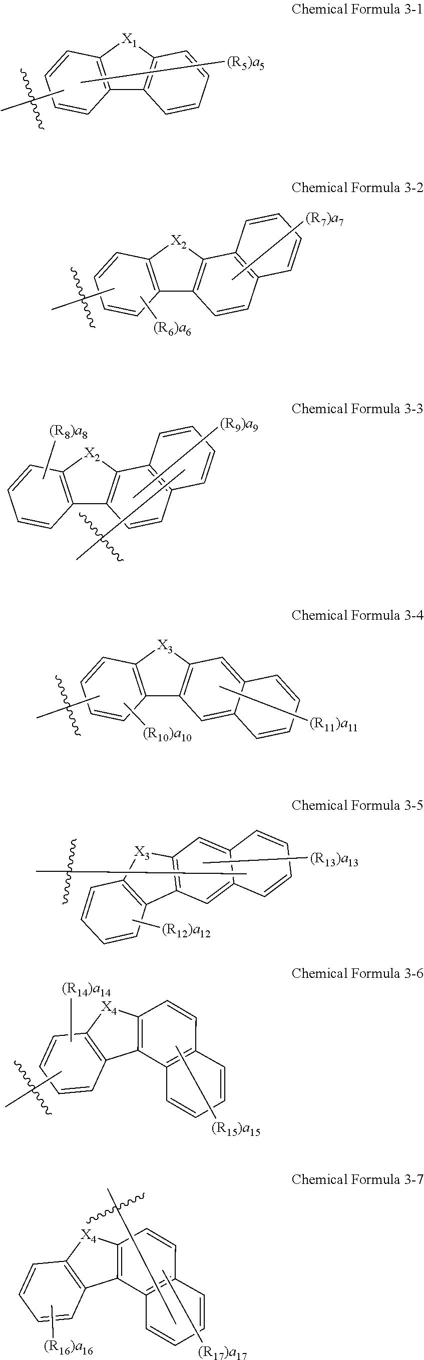

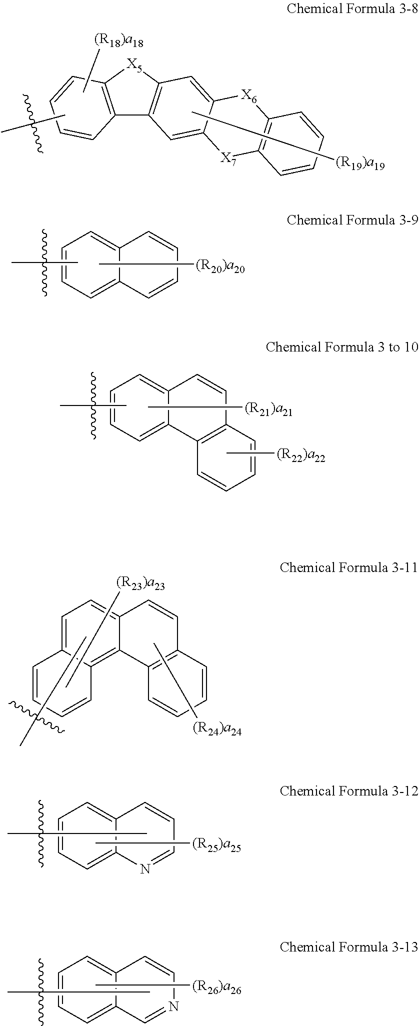

Wherein, X₁ to X₅ are each independently selected from O, S, C(R)₂, N(R), or Si(R)₂;
X₆ to X₇ are each independently selected from a single bond, O, S, C(R)₂, N(R), or Si(R)₂, with the proviso that X₆ and X₇ are not simultaneously a single bond;
R is selected from the group consisting of hydrogen, deuterium, fluorine, chlorine, substituted or unsubstituted alkyl having 1 to 12 carbon atoms, substituted or unsubstituted haloalkyl having 1 to 12 carbon atoms, substituted or unsubstituted alkoxy having 1 to 12 carbon atoms, substituted or unsubstituted cycloalkyl having 3 to 10 carbon atoms, substituted or unsubstituted aryl having 6 to 60 carbon atoms, and substituted or unsubstituted heteroaryl having 3 to 60 carbon atoms;
R₅ to R₂₆ are the same or different from each other and are each independently selected from the group consisting of deuterium, fluorine, chlorine, substituted or unsubstituted alkyl having 1 to 12 carbon atoms, substituted or unsubstituted haloalkyl having 1 to 12 carbon atoms, substituted or unsubstituted alkoxy having 1 to 12 carbon atoms, substituted or unsubstituted cycloalkyl having 3 to 10 carbon atoms, substituted or unsubstituted aryl having 6 to 60 carbon atoms, and substituted or unsubstituted heteroaryl having 3 to 60 carbon atoms;
a₅ is an integer from 0 to 7; when a₅ is greater than 1, any two R₅ may be the same or different;
a₆ is an integer from 0 to 3; when a₆ is greater than 1, any two R₆ may be the same or different;
a₇ is an integer from 0 to 6; when a₇ is greater than 1, any two R₇ may be the same or different;
a₈ is an integer from 0 to 4; when a₈ is greater than 1, any two R₈ may be the same or different;
a₉ is an integer from 0 to 5; when a₉ is greater than 1, any two R₉ may be the same or different;
a₁₀ is an integer from 0 to 3; when a₁₀ is greater than 1, any two R₁₀ may be the same or different;
a₁₁ is an integer from 0 to 6; when a₁₁ is greater than 1, any two Ru may be the same or different;
a₁₂ is an integer from 0 to 4; when a₁₂ is greater than 1, any two R₁₂ may be the same or different;
a₁₃ is an integer from 0 to 5; when a₁₃ is greater than 1, any two R₁₃ may be the same or different;
a₁₄ is an integer from 0 to 3; when a₁₄ is greater than 1, any two R₁₄ may be the same or different;
a₁₅ is an integer from 0 to 6; when a₁₅ is greater than 1, any two R₁₅ may be the same or different;
a₁₆ is an integer from 0 to 4; when a₁₆ is greater than 1, any two R₁₆ may be the same or different;
a₁₇ is an integer from 0 to 5; when a₁₇ is greater than 1, any two R₁₇ may be the same or different;
a₁₈ is an integer from 0 to 3; when a₁₈ is greater than 1, any two R₁₈ may be the same or different;
a₁₉ is an integer from 0 to 6; when a₁₉ is greater than 1, any two R₁₉ may be the same or different;
a₂₀ is an integer from 0 to 7; when a₂₀ is greater than 1, any two R₂₀ may be the same or different;
a₂₁ is an integer from 0 to 5; when a₂₁ is greater than 1, any two R₂₁ may be the same or different;
a₂₂ is an integer from 0 to 4; when a₂₂ is greater than 1, any two R₂₂ may be the same or different;
a₂₃ is an integer from 0 to 5; when a₂₃ is greater than 1, any two R₂₃ may be the same or different;
a₂₄ is an integer from 0 to 6; when a₂₄ is greater than 1, any two R₂₄ may be the same or different;
a₂₅ is an integer from 0 to 6; when a₂₅ is greater than 1, any two R₂₅ may be the same or different;
a₂₆ is an integer from 0 to 6; when a₂₆ is greater than 1, any two R₂₆ may be the same or different.

The groups represented by Chemical Formulas 3-1 to 3-13 all possess planar conjugated structures. When these structures are introduced into Chemical Formula 1 and connected to the anthracene group, they enable the compound to form an extended conjugated system, which facilitates improved charge carrier mobility and consequently enhances device efficiency.

In some embodiments, group Q features a dibenzoheterocyclic structure. For example, when Q is selected from a group consisting of the groups represented by Chemical Formulas 3-1 to 3-8 and X₁ to X₅ are each independently selected from O, S, or N(R), the dibenzoheterocyclic group Q can form effective conjugation with the anthracene core. This extends the conjugation between electron-withdrawing and electron-donating moieties, endowing the compound with enhanced charge mobility for more efficient electron transport, thereby improving device efficiency. Furthermore, the dibenzoheterocyclic group Q containing heteroatoms (such as O, S, or N) imparts significant polarity to the molecule. For instance, when this compound is used as a blue light host material, it improves the energy level alignment at interfaces with adjacent functional layers (e.g., light-emitting auxiliary layers or hole-blocking layers). This enhances the injection properties of the blue host material, strengthens interfacial interactions with adjacent layers, and reduces the operating voltage of the device.

In certain embodiment, Q is selected from the group consisting of a group represented by Chemical Formula 3-1, a group represented by Chemical Formula 3-2, a group represented by Chemical Formula 3-4, a group represented by Chemical Formula 3-6, a group represented by Chemical Formula 3-8a, a group represented by Chemical Formula 3-8b, a group represented by Chemical Formula 3-9, a group represented by Chemical Formula 3-10, a group represented by Chemical Formula 3-11a, a group represented by Chemical Formula 3-12a, and a group represented by Chemical Formula 3-13;

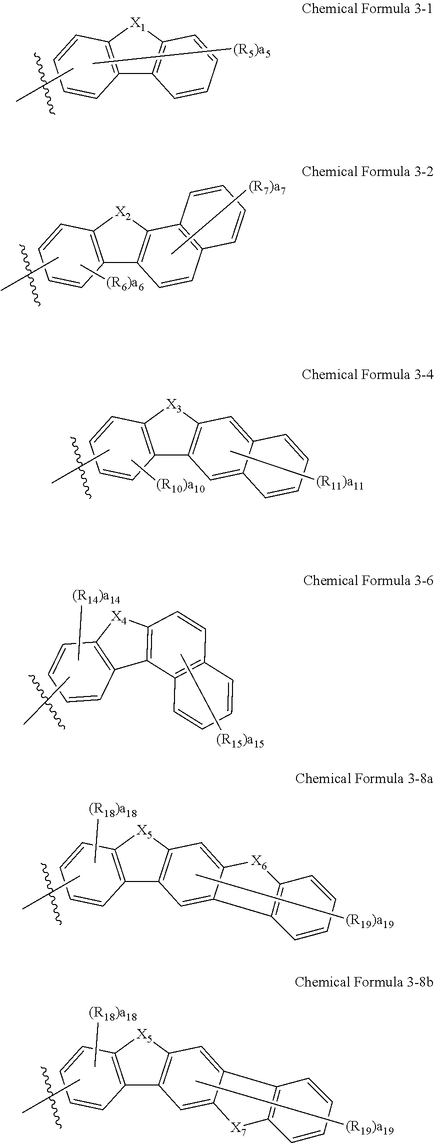

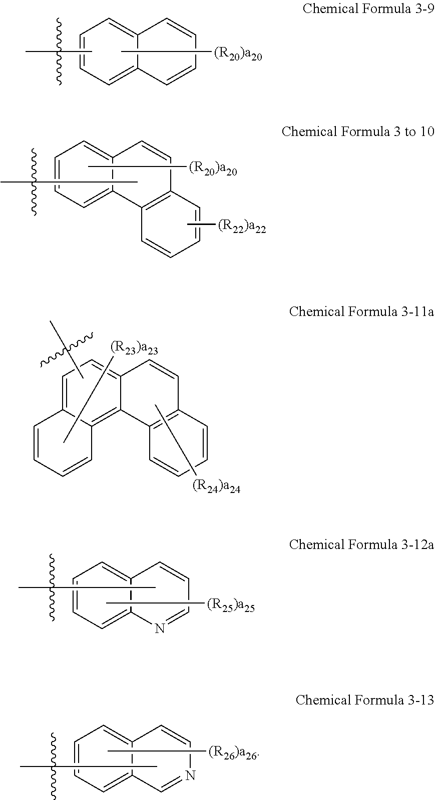

Thus, by selecting the connection position between group Q and L₂, greater steric competition can be created between group Q and the anthracene moiety or L₂, thereby further enhancing the three-dimensional character of the compound represented by Chemical Formula 1 and further reducing various issues caused by excessive planarity of the compound, such as easy crystallization and overly strong intermolecular interactions.

In one embodiment of the present disclosure, X₁ to X₅ are each independently selected from O or S; X₆ to X₇ are each independently selected from a single bond, O, or S, with the proviso that X₆ and X₇ are not simultaneously a single bond.

In one embodiment of the present disclosure, R₅ to R₂₆ are each independently selected from the group consisting of deuterium, fully deuterated or non-deuterated methyl, and aryl groups, wherein the aryl groups are selected from fully deuterated or non-deuterated phenyl, fully deuterated or non-deuterated biphenyl, fully deuterated or non-deuterated naphthyl, fully deuterated or non-deuterated anthryl, fully deuterated or non-deuterated phenanthryl, and groups formed by connecting any two or three of the above via single bonds.

In one exemplary embodiment, R₅ to R₂₆ are each independently selected from the group consisting of deuterium, fully deuterated methyl, and aryl groups, wherein the aryl groups are selected from fully deuterated phenyl, fully deuterated biphenyl, fully deuterated naphthyl, fully deuterated anthryl, fully deuterated phenanthryl, and groups formed by connecting any two or three of the above via single bonds. In this way, the deuteration of the Q group can enhance the electrical stability of the compound, improve its durability and stability, and thereby increase the lifetime of devices containing the compound.

In one embodiment of the present disclosure, L₁ and L₂ are each independently selected from a single bond, fully deuterated or non-deuterated phenylene, fully deuterated or non-deuterated biphenylene, fully deuterated or non-deuterated naphthylene, fully deuterated or non-deuterated anthrylene, fully deuterated or non-deuterated phenanthrylene, and groups formed by connecting any two or three of the above via single bonds.

For example, L₁ is selected from fully deuterated phenylene, fully deuterated biphenylene, fully deuterated naphthylene, fully deuterated anthrylene, fully deuterated phenanthrylene, and groups formed by connecting any two or three of the above via single bonds. In this way, deuteration (D-substitution) of the L₁ group can enhance the electrical stability of the compound, improve its durability and stability, and thereby increase the lifetime of devices containing the compound.

For example, L₂ is selected from fully deuterated phenylene, fully deuterated biphenylene, fully deuterated naphthylene, fully deuterated anthrylene, fully deuterated phenanthrylene, and groups formed by connecting any two or three of the above via single bonds. In this way, deuteration (D-substitution) of the L₂ group can enhance the electrical stability of the compound, improve its durability and stability, and thereby increase the lifetime of devices containing the compound.

In one embodiment of the present disclosure, R₁ to R₃ are all deuterium; a₁ is 0 or 4; a₂ is 0 or 4; a₃ is 0 or 3; and a₁ to a₃ are simultaneously 0, or simultaneously non-zero. In other words, the triptycene moiety is either fully deuterated or unsubstituted. This can reduce the synthesis difficulty of the compound. When a₁ is 4, a₂ is 4, and a₃ is 3, the triptycene is fully deuterated, which can enhance the electrical stability of the compound, improve its durability and stability, thereby increasing the lifetime of devices containing this compound.

In one embodiment of the present disclosure, R₄ is deuterium; a₄ is 0 or 8. In other words, the anthracene moiety is either fully deuterated or unsubstituted. This can reduce the synthesis difficulty of the compound. When a₄ is 8, the anthracene is fully deuterated, which can enhance the electrical stability of the compound, improve its durability and stability, thereby increasing the lifetime of devices containing this compound.

As an exemplary application, the compound may serve as a blue light host material. Typically, the host material has a T1 energy level lower than that of the guest dopant (guest material), enabling efficient Triplet-Triplet Fusion (TTF) in the host material. That is, the triplet excitons on the host material do not transfer to the dopant but instead efficiently collide with each other on the host material to produce singlet excitons, which then transfer energy to the guest material through energy transfer, causing the guest material to emit light radiatively, thereby improving device efficiency. The blue host material of the present disclosure employs deuteration on the anthracene moiety, which can improve device lifetime. Empirically, TTF primarily occurs in the host material. However, when triplet excitons collide, they generate high-energy intermediates. If the energy of these intermediates exceeds the bond dissociation energy of the material itself (typically at active sites where bonds are most prone to breakage), it may lead to degradation of the organic material. By strategically deuterating the host material within a certain range, the electrical stability of the material can be enhanced, improving the tolerance and stability of host materials, thereby extending device lifetime.

In one embodiment of the present disclosure, the compound is selected from the group consisting of the following compounds:

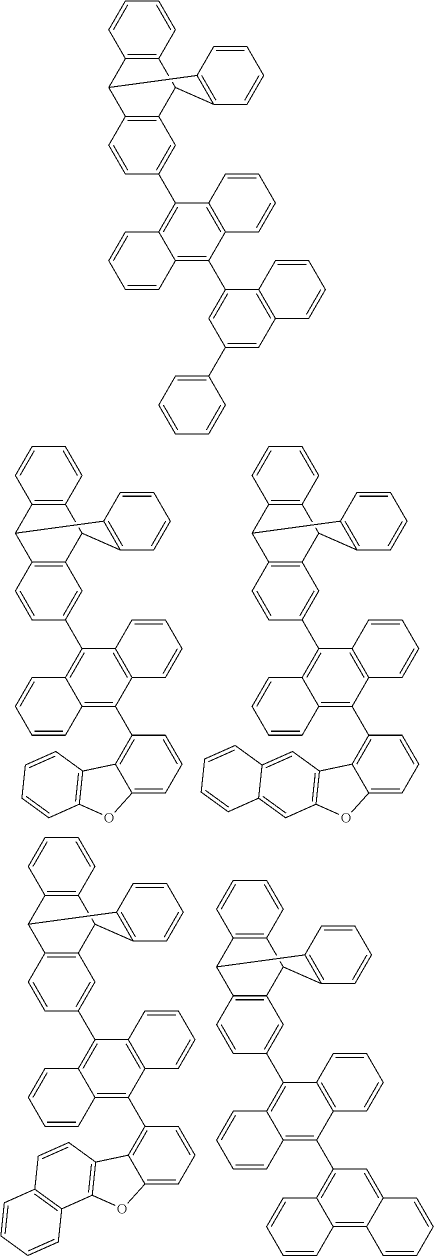

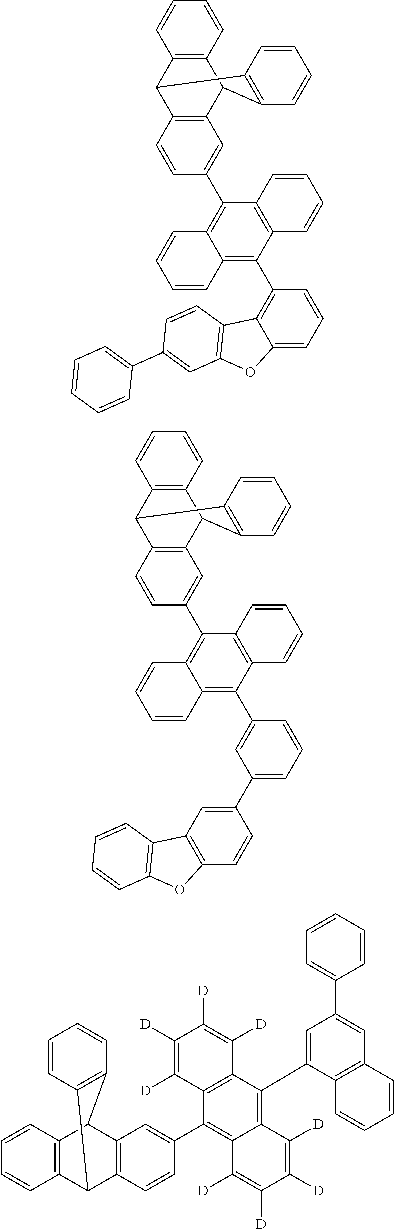

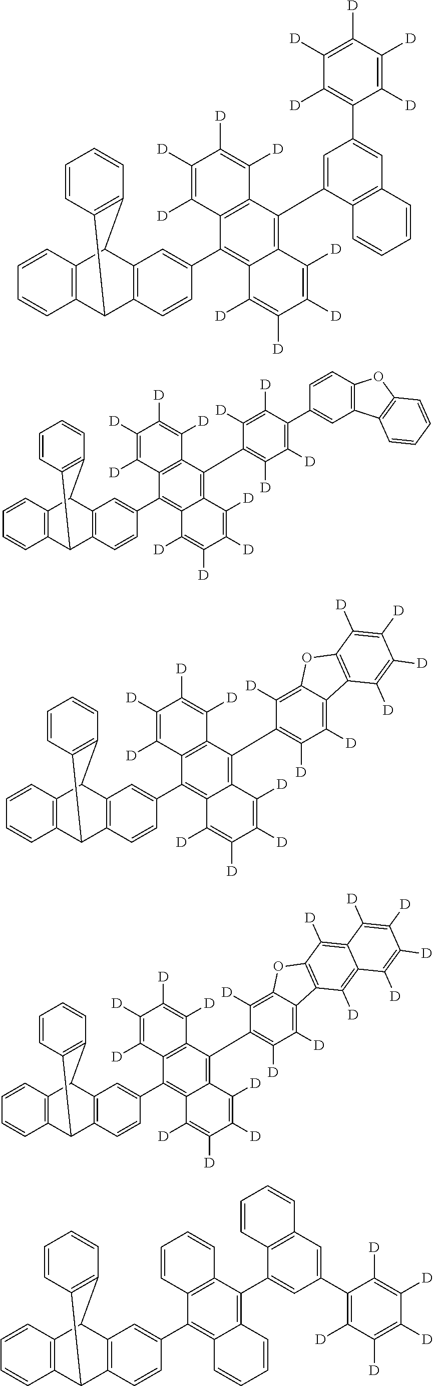

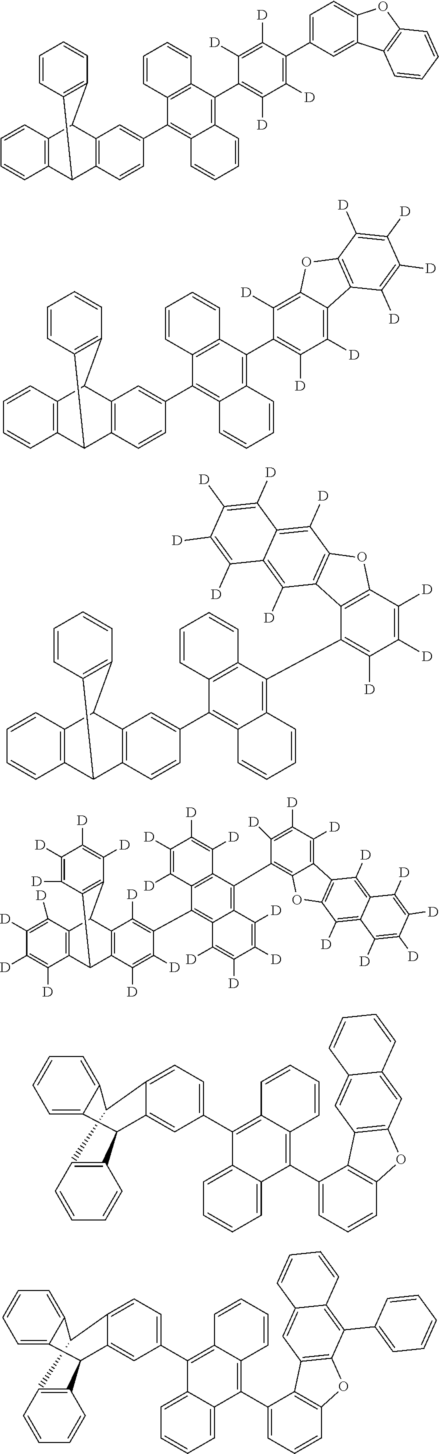

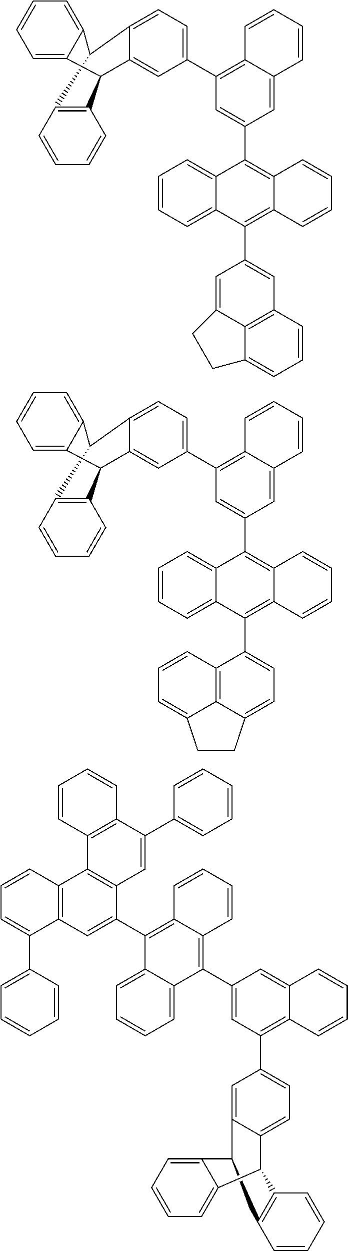

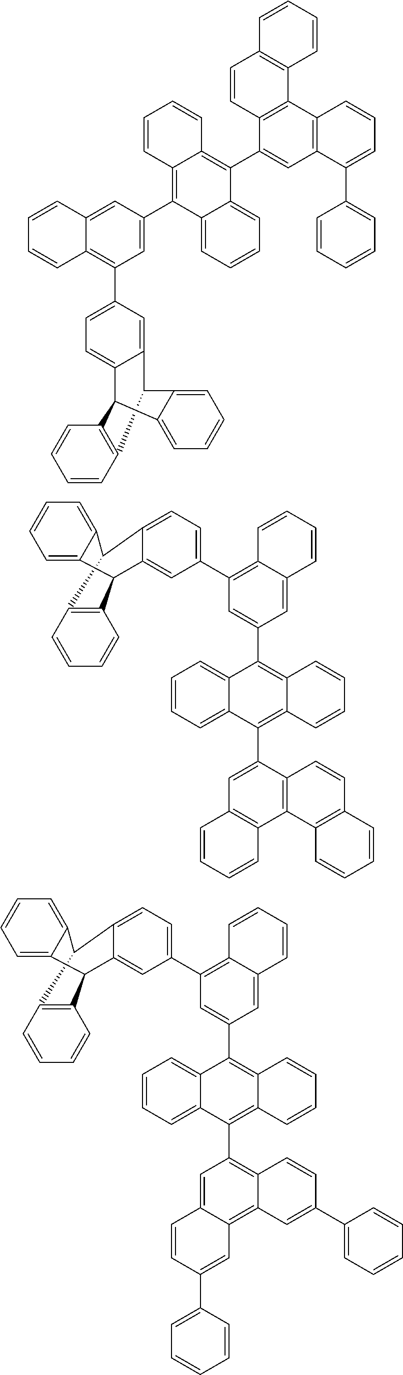

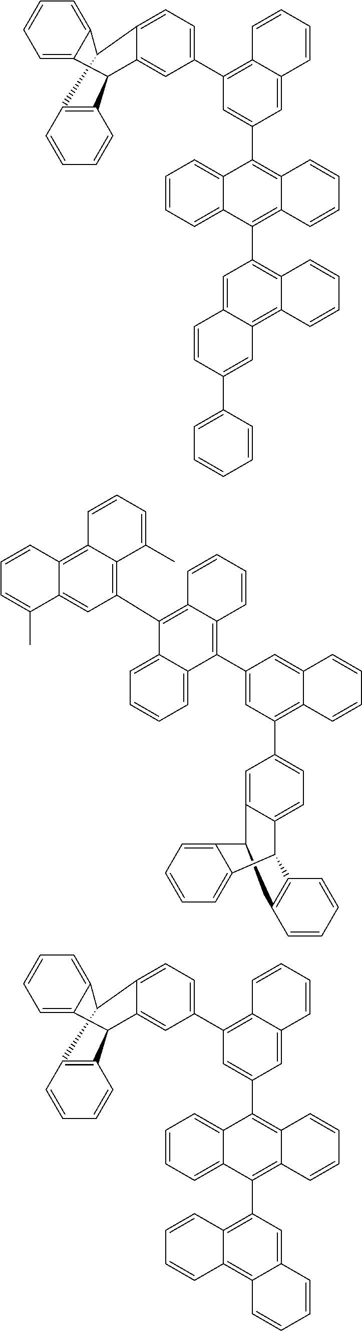

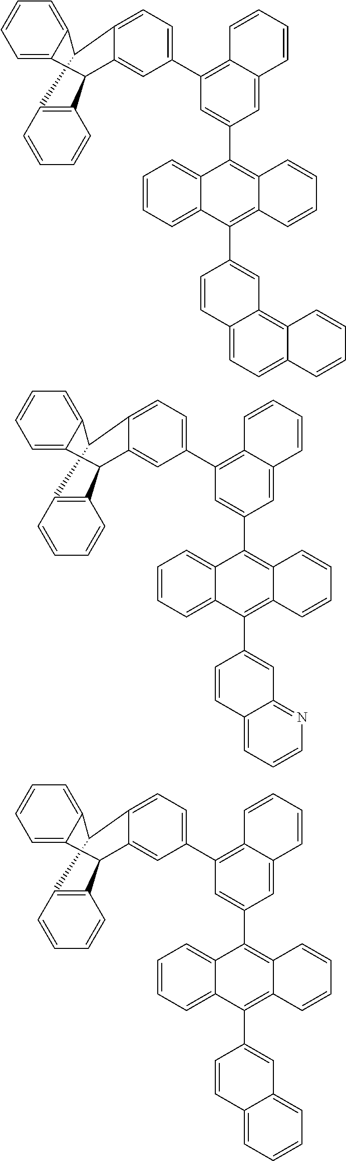

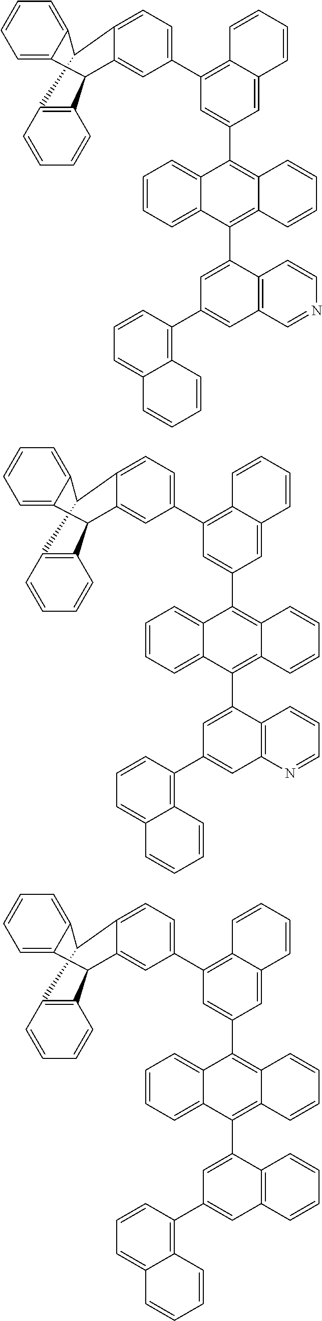

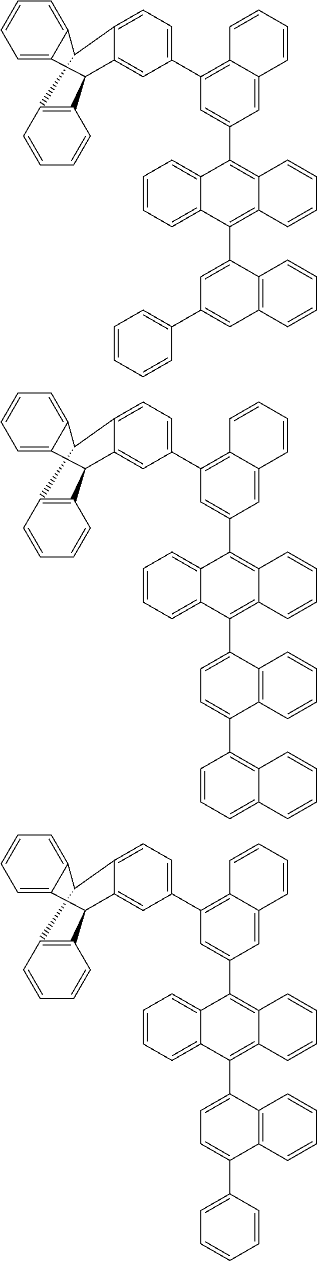

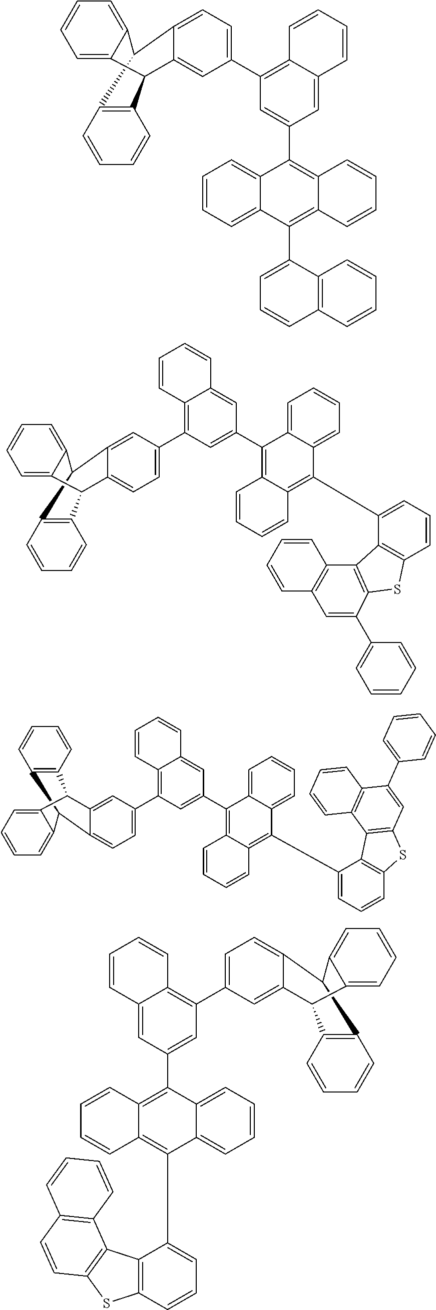

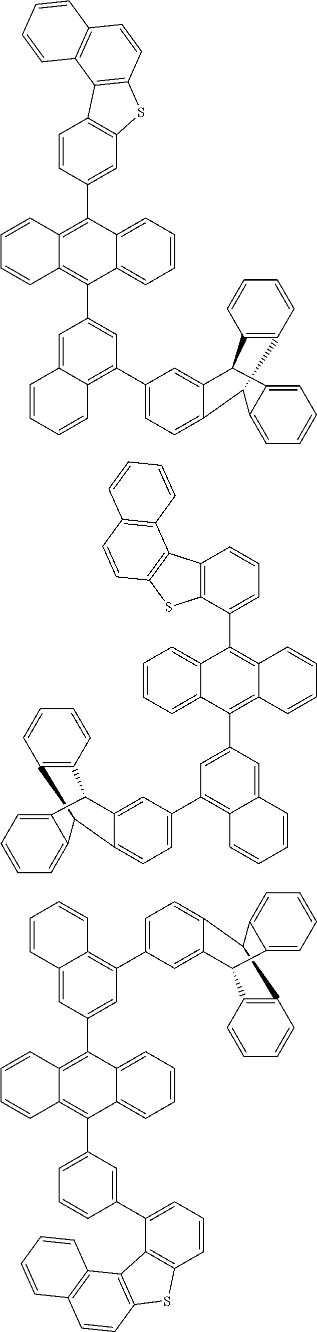

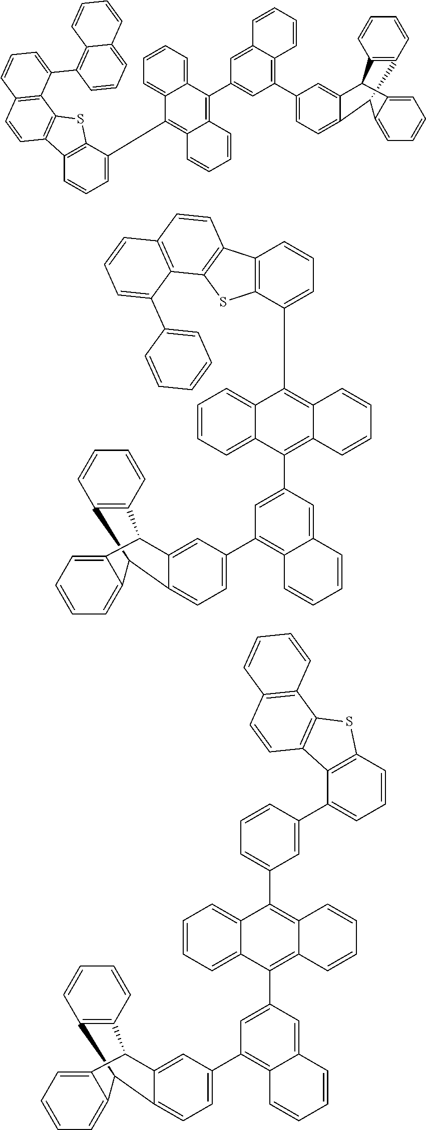

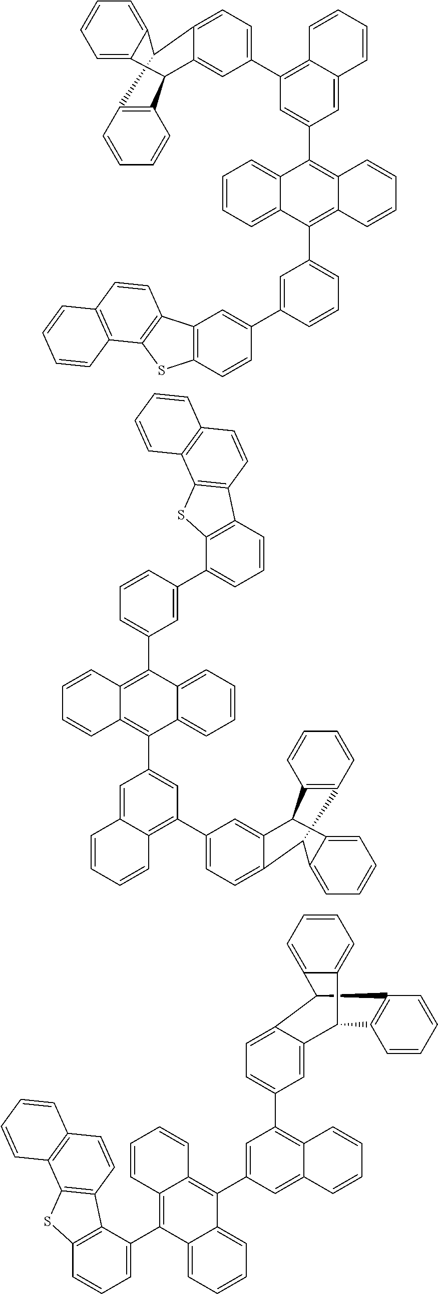

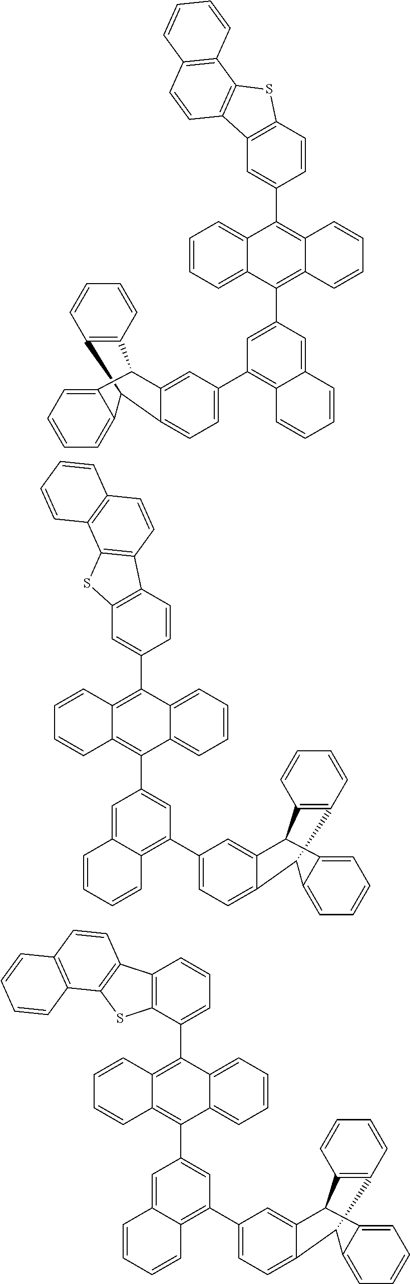

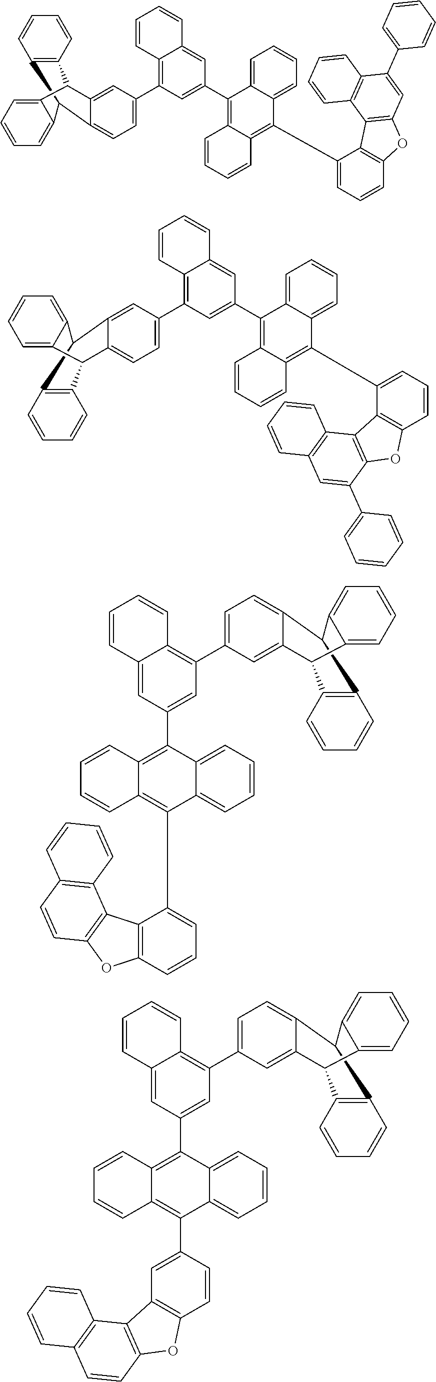

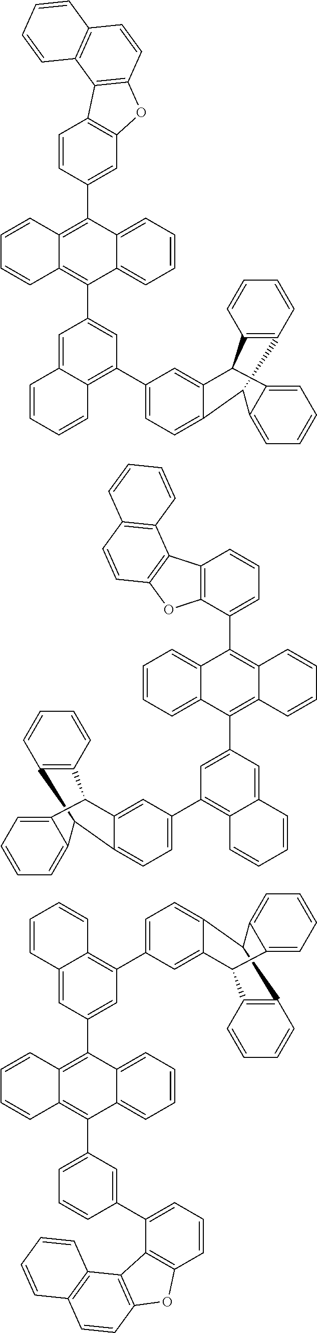

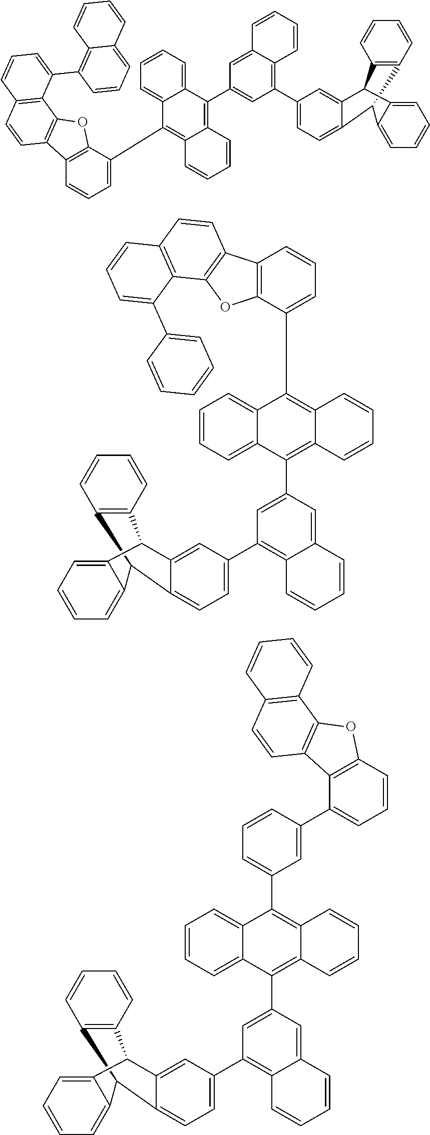

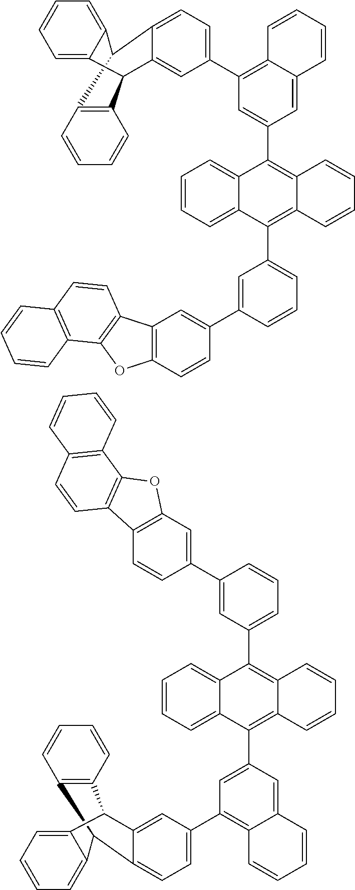

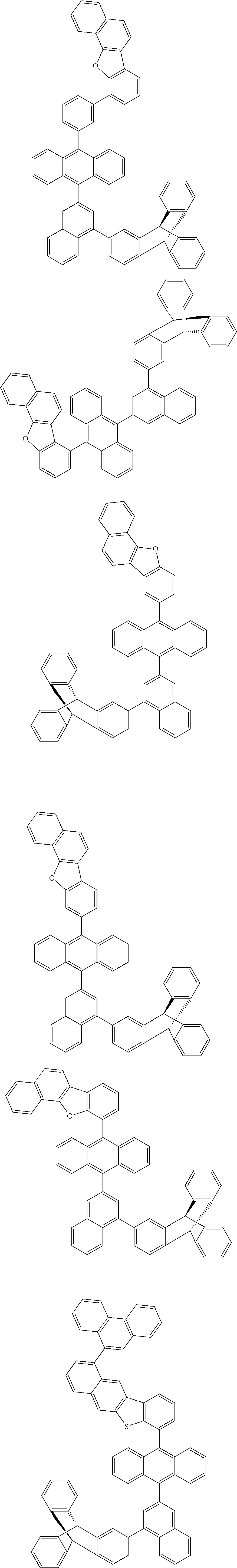

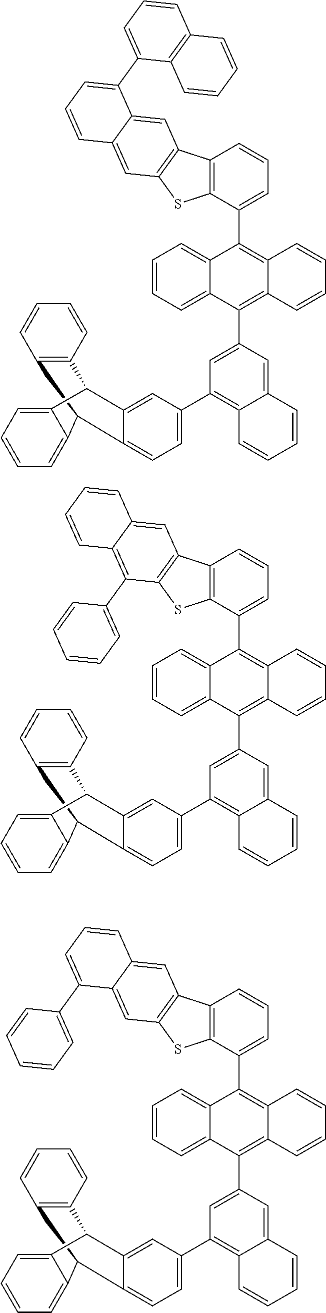

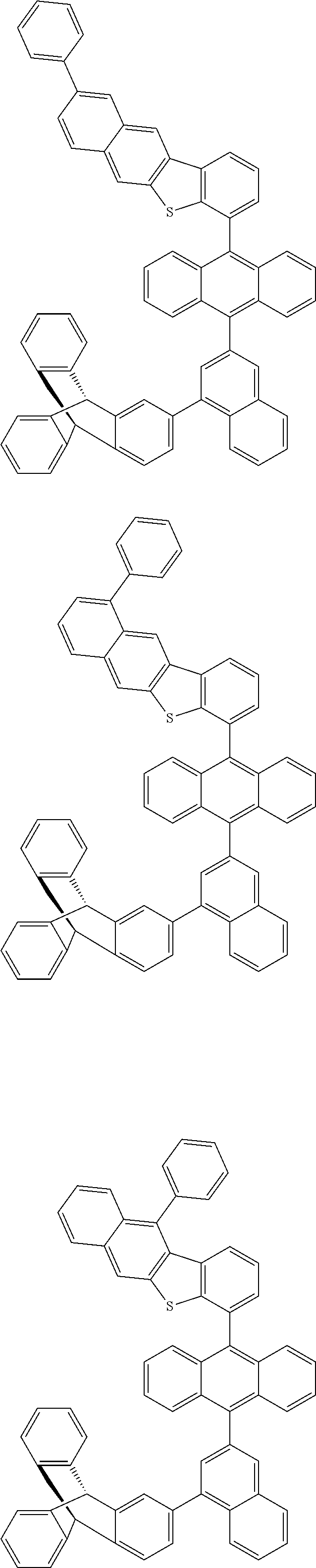 [SMILES: c1ccc(-c2ccc3cc4sc5c(-c6c7ccccc7c(-c7cc(-c8ccc9c(c8)[C@H]8c%10ccccc%10[C@@H]9c9ccccc98)c8ccccc8c7)c7ccccc67)cccc5c4cc3c2)cc1 ; c1ccc(-c2cccc3cc4sc5c(-c6c7ccccc7c(-c7cc(-c8ccc9c(c8)[C@H]8c%10ccccc%10[C@@H]9c9ccccc98)c8ccccc8c7)c7ccccc67)cccc5c4cc23)cc1 ; c1ccc(-c2c3ccccc3cc3sc4c(-c5c6ccccc6c(-c6cc(-c7ccc8c(c7)[C@H]7c9ccccc9[C@@H]8c8ccccc87)c7ccccc7c6)c6ccccc56)cccc4c23)cc1]

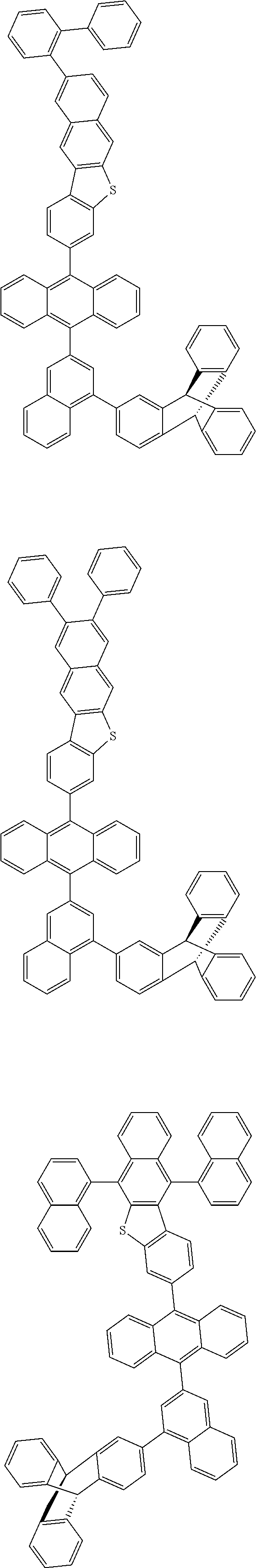 [SMILES: c1ccc(-c2ccccc2-c2ccc3cc4sc5cc(-c6c7ccccc7c(-c7cc(-c8ccc9c(c8)[C@H]8c%10ccccc%10[C@@H]9c9ccccc98)c8ccccc8c7)c7ccccc67)ccc5c4cc3c2)cc1 ; c1ccc(-c2cc3cc4sc5cc(-c6c7ccccc7c(-c7cc(-c8ccc9c(c8)[C@H]8c%10ccccc%10[C@@H]9c9ccccc98)c8ccccc8c7)c7ccccc67)ccc5c4cc3cc2-c2ccccc2)cc1 ; c1ccc2c(-c3ccc4c(c3)[C@H]3c5ccccc5[C@@H]4c4ccccc43)cc(-c3c4ccccc4c(-c4ccc5c(c4)sc4c(-c6cccc7ccccc67)c6ccccc6c(-c6cccc7ccccc67)c45)c4ccccc34)cc2c1]

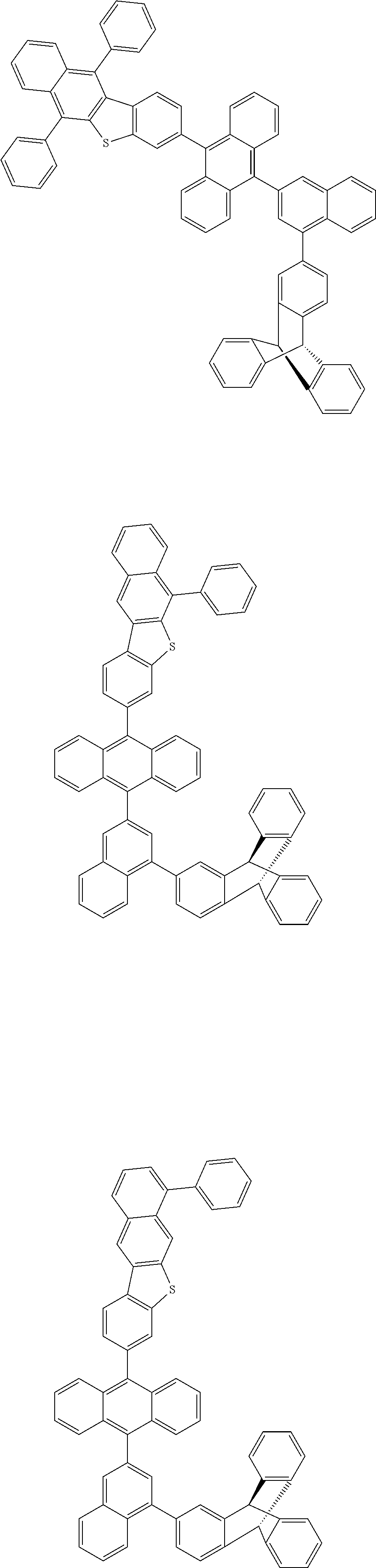 [SMILES: c1ccc(-c2c3ccccc3c(-c3ccccc3)c3c2sc2cc(-c4c5ccccc5c(-c5cc(-c6ccc7c(c6)[C@H]6c8ccccc8[C@@H]7c7ccccc76)c6ccccc6c5)c5ccccc45)ccc23)cc1 ; c1ccc(-c2c3ccccc3cc3c2sc2cc(-c4c5ccccc5c(-c5cc(-c6ccc7c(c6)[C@H]6c8ccccc8[C@@H]7c7ccccc76)c6ccccc6c5)c5ccccc45)ccc23)cc1 ; c1ccc(-c2cccc3cc4c(cc23)sc2cc(-c3c5ccccc5c(-c5cc(-c6ccc7c(c6)[C@H]6c8ccccc8[C@@H]7c7ccccc76)c6ccccc6c5)c5ccccc35)ccc24)cc1]

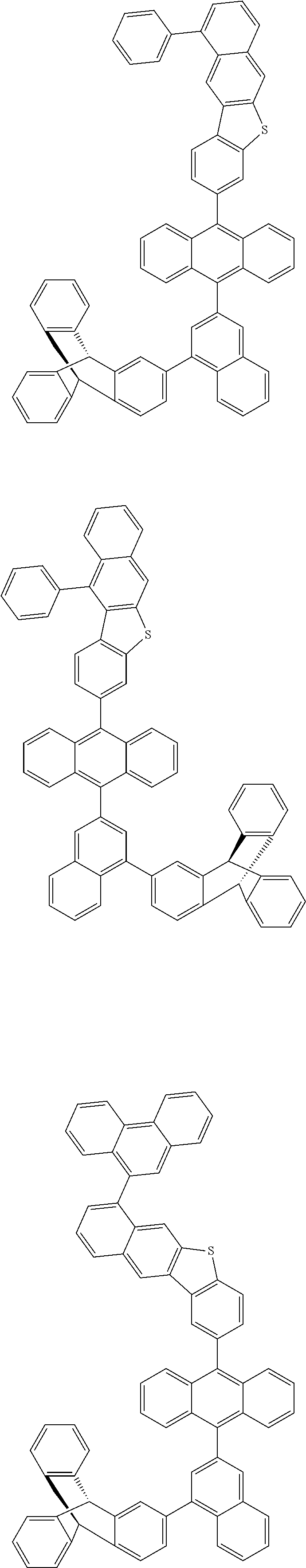 [SMILES: c1ccc(-c2cccc3cc4sc5cc(-c6c7ccccc7c(-c7cc(-c8ccc9c(c8)[C@H]8c%10ccccc%10[C@@H]9c9ccccc98)c8ccccc8c7)c7ccccc67)ccc5c4cc23)cc1 ; c1ccc(-c2c3ccccc3cc3sc4cc(-c5c6ccccc6c(-c6cc(-c7ccc8c(c7)[C@H]7c9ccccc9[C@@H]8c8ccccc87)c7ccccc7c6)c6ccccc56)ccc4c23)cc1 ; c1ccc2c(-c3ccc4c(c3)[C@H]3c5ccccc5[C@@H]4c4ccccc43)cc(-c3c4ccccc4c(-c4ccc5sc6cc7c(-c8cc9ccccc9c9ccccc89)cccc7cc6c5c4)c4ccccc34)cc2c1]

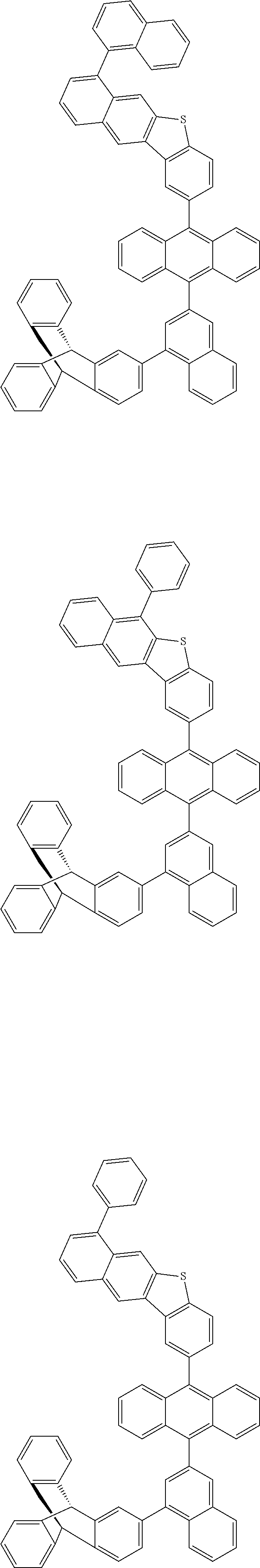 [SMILES: c1ccc2c(-c3ccc4c(c3)[C@H]3c5ccccc5[C@@H]4c4ccccc43)cc(-c3c4ccccc4c(-c4ccc5sc6cc7c(-c8cccc9ccccc89)cccc7cc6c5c4)c4ccccc34)cc2c1 ; c1ccc(-c2c3ccccc3cc3c2sc2ccc(-c4c5ccccc5c(-c5cc(-c6ccc7c(c6)[C@H]6c8ccccc8[C@@H]7c7ccccc76)c6ccccc6c5)c5ccccc45)cc23)cc1 ; c1ccc(-c2cccc3cc4c(cc23)sc2ccc(-c3c5ccccc5c(-c5cc(-c6ccc7c(c6)[C@H]6c8ccccc8[C@@H]7c7ccccc76)c6ccccc6c5)c5ccccc35)cc24)cc1]

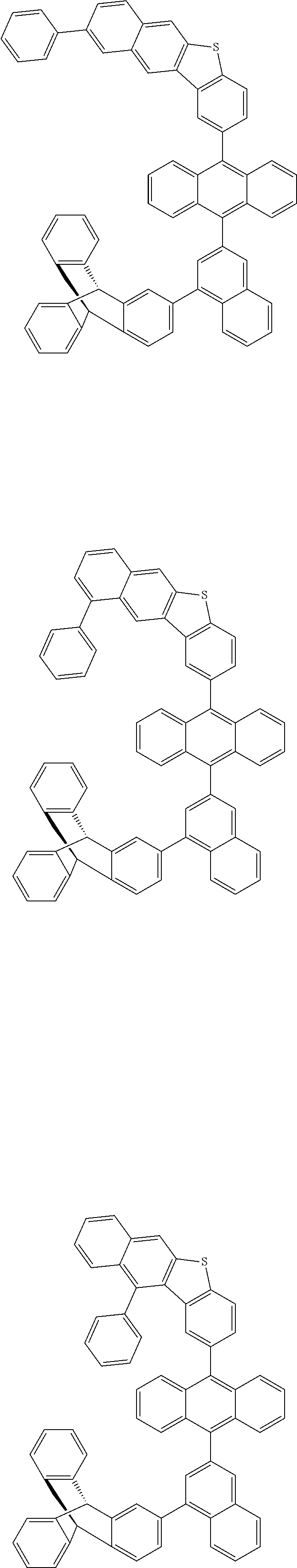 [SMILES: c1ccc(-c2ccc3cc4sc5ccc(-c6c7ccccc7c(-c7cc(-c8ccc9c(c8)[C@H]8c%10ccccc%10[C@@H]9c9ccccc98)c8ccccc8c7)c7ccccc67)cc5c4cc3c2)cc1 ; c1ccc(-c2cccc3cc4sc5ccc(-c6c7ccccc7c(-c7cc(-c8ccc9c(c8)[C@H]8c%10ccccc%10[C@@H]9c9ccccc98)c8ccccc8c7)c7ccccc67)cc5c4cc23)cc1 ; c1ccc(-c2c3ccccc3cc3sc4ccc(-c5c6ccccc6c(-c6cc(-c7ccc8c(c7)[C@H]7c9ccccc9[C@@H]8c8ccccc87)c7ccccc7c6)c6ccccc56)cc4c23)cc1]

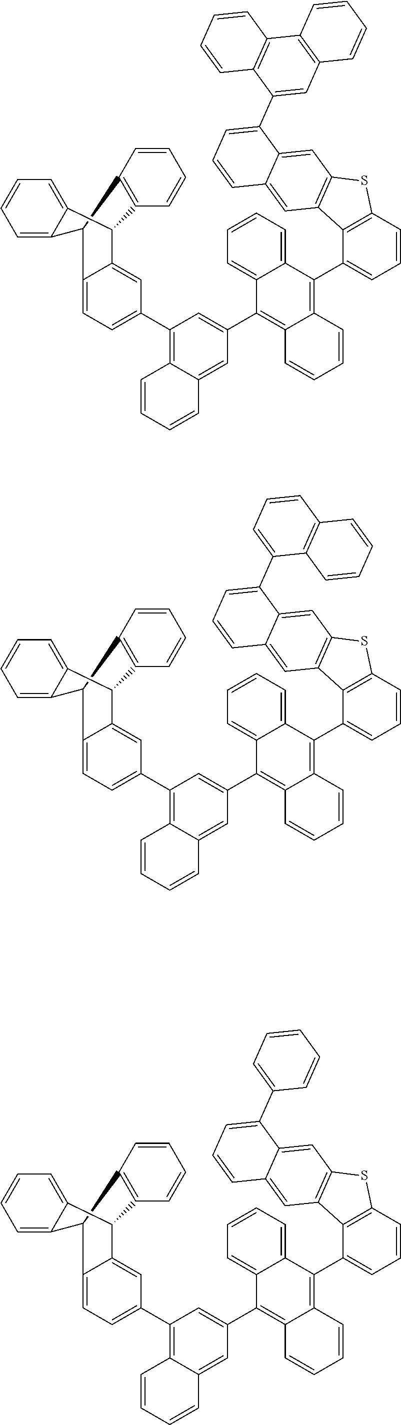 [SMILES: c1ccc2c(-c3ccc4c(c3)[C@H]3c5ccccc5[C@@H]4c4ccccc43)cc(-c3c4ccccc4c(-c4cccc5sc6cc7c(-c8cc9ccccc9c9ccccc89)cccc7cc6c45)c4ccccc34)cc2c1 ; c1ccc2c(-c3ccc4c(c3)[C@H]3c5ccccc5[C@@H]4c4ccccc43)cc(-c3c4ccccc4c(-c4cccc5sc6cc7c(-c8cccc9ccccc89)cccc7cc6c45)c4ccccc34)cc2c1 ; c1ccc(-c2cccc3cc4c(cc23)sc2cccc(-c3c5ccccc5c(-c5cc(-c6ccc7c(c6)[C@H]6c8ccccc8[C@@H]7c7ccccc76)c6ccccc6c5)c5ccccc35)c24)cc1]

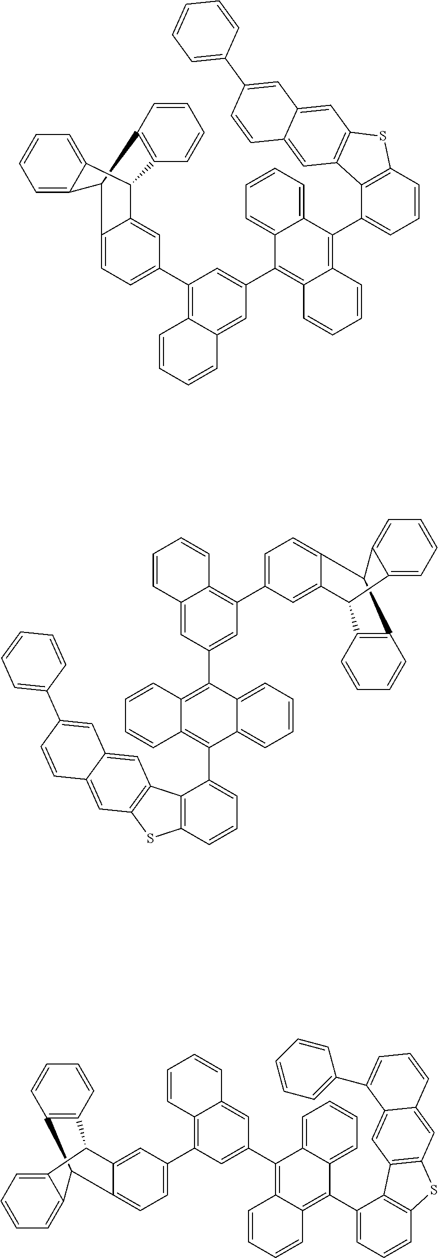 [SMILES: c1ccc(-c2ccc3cc4c(cc3c2)sc2cccc(-c3c5ccccc5c(-c5cc(-c6ccc7c(c6)[C@H]6c8ccccc8[C@@H]7c7ccccc76)c6ccccc6c5)c5ccccc35)c24)cc1 ; c1ccc(-c2ccc3cc4sc5cccc(-c6c7ccccc7c(-c7cc(-c8ccc9c(c8)[C@H]8c%10ccccc%10[C@@H]9c9ccccc98)c8ccccc8c7)c7ccccc67)c5c4cc3c2)cc1 ; c1ccc(-c2cccc3cc4sc5cccc(-c6c7ccccc7c(-c7cc(-c8ccc9c(c8)[C@H]8c%10ccccc%10[C@@H]9c9ccccc98)c8ccccc8c7)c7ccccc67)c5c4cc23)cc1]

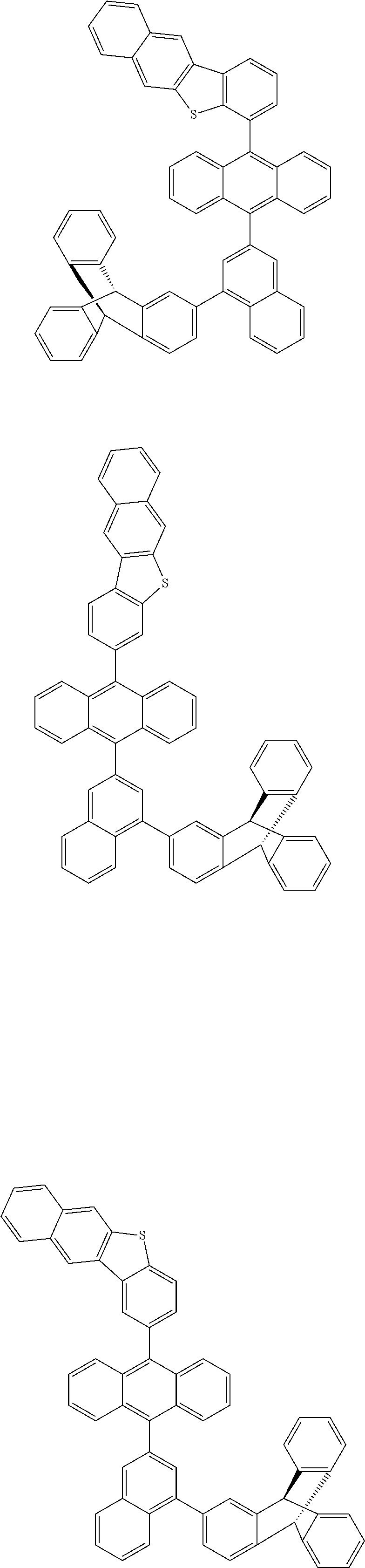 [SMILES: c1ccc2cc3c(cc2c1)sc1c(-c2c4ccccc4c(-c4cc(-c5ccc6c(c5)[C@H]5c7ccccc7[C@@H]6c6ccccc65)c5ccccc5c4)c4ccccc24)cccc13 ; c1ccc2cc3c(cc2c1)sc1cc(-c2c4ccccc4c(-c4cc(-c5ccc6c(c5)[C@H]5c7ccccc7[C@@H]6c6ccccc65)c5ccccc5c4)c4ccccc24)ccc13 ; c1ccc2cc3c(cc2c1)sc1ccc(-c2c4ccccc4c(-c4cc(-c5ccc6c(c5)[C@H]5c7ccccc7[C@@H]6c6ccccc65)c5ccccc5c4)c4ccccc24)cc13]

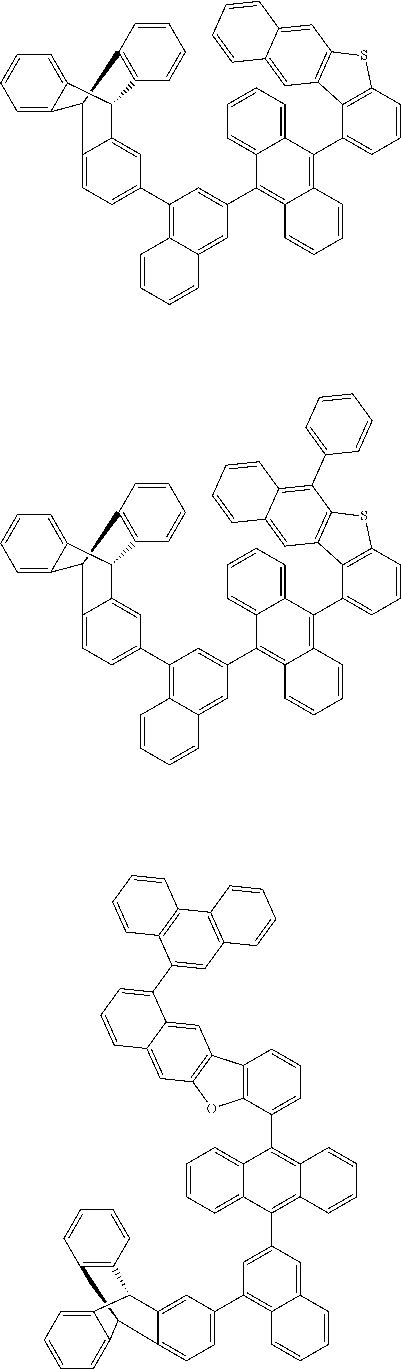 [SMILES: c1ccc2cc3c(cc2c1)sc1cccc(-c2c4ccccc4c(-c4cc(-c5ccc6c(c5)[C@H]5c7ccccc7[C@@H]6c6ccccc65)c5ccccc5c4)c4ccccc24)c13 ; c1ccc(-c2c3ccccc3cc3c2sc2cccc(-c4c5ccccc5c(-c5cc(-c6ccc7c(c6)[C@H]6c8ccccc8[C@@H]7c7ccccc76)c6ccccc6c5)c5ccccc45)c23)cc1 ; c1ccc2c(-c3ccc4c(c3)[C@H]3c5ccccc5[C@@H]4c4ccccc43)cc(-c3c4ccccc4c(-c4cccc5c4oc4cc6cccc(-c7cc8ccccc8c8ccccc78)c6cc45)c4ccccc34)cc2c1]

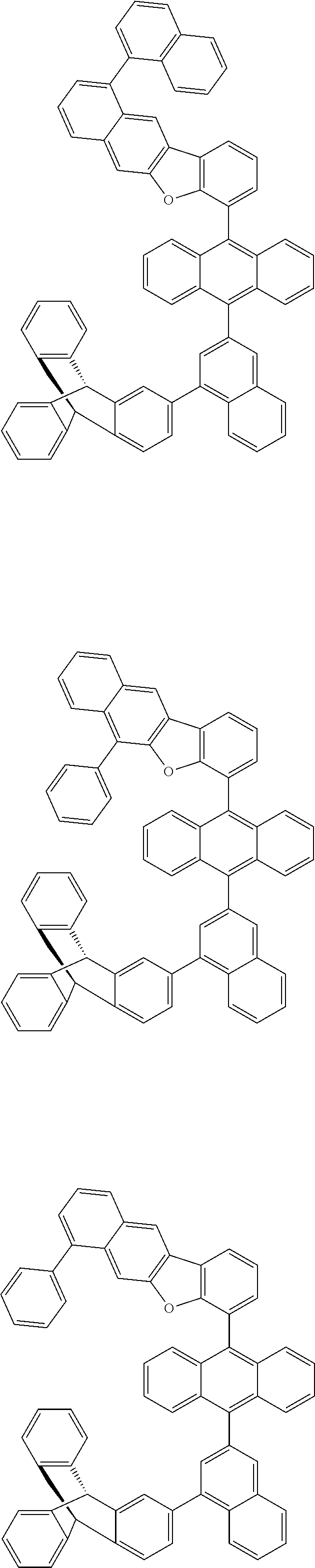 [SMILES: c1ccc2c(-c3ccc4c(c3)[C@H]3c5ccccc5[C@@H]4c4ccccc43)cc(-c3c4ccccc4c(-c4cccc5c4oc4cc6cccc(-c7cccc8ccccc78)c6cc45)c4ccccc34)cc2c1 ; c1ccc(-c2c3ccccc3cc3c2oc2c(-c4c5ccccc5c(-c5cc(-c6ccc7c(c6)[C@H]6c8ccccc8[C@@H]7c7ccccc76)c6ccccc6c5)c5ccccc45)cccc23)cc1 ; c1ccc(-c2cccc3cc4c(cc23)oc2c(-c3c5ccccc5c(-c5cc(-c6ccc7c(c6)[C@H]6c8ccccc8[C@@H]7c7ccccc76)c6ccccc6c5)c5ccccc35)cccc24)cc1]

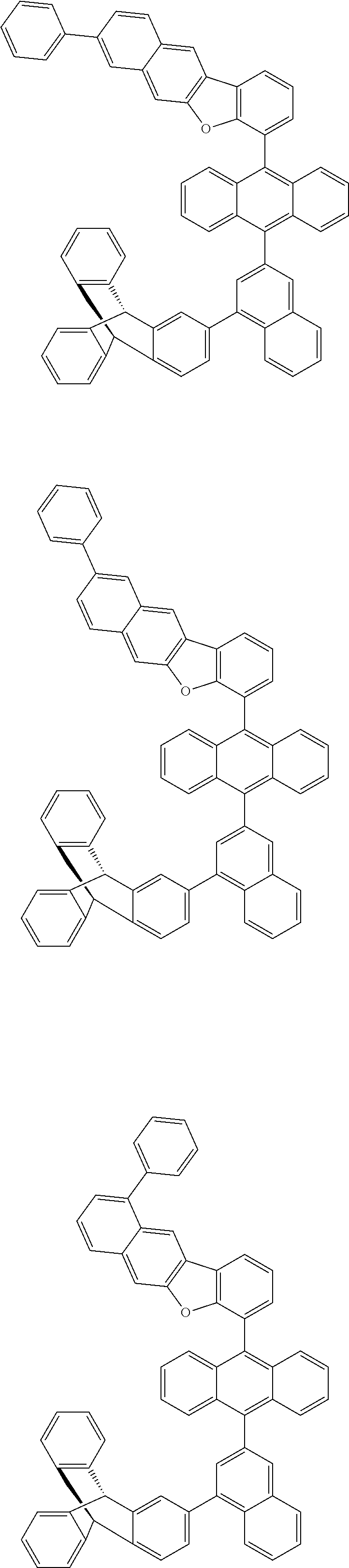 [SMILES: c1ccc(-c2ccc3cc4c(cc3c2)oc2c(-c3c5ccccc5c(-c5cc(-c6ccc7c(c6)[C@H]6c8ccccc8[C@@H]7c7ccccc76)c6ccccc6c5)c5ccccc35)cccc24)cc1 ; c1ccc(-c2ccc3cc4oc5c(-c6c7ccccc7c(-c7cc(-c8ccc9c(c8)[C@H]8c%10ccccc%10[C@@H]9c9ccccc98)c8ccccc8c7)c7ccccc67)cccc5c4cc3c2)cc1 ; c1ccc(-c2cccc3cc4oc5c(-c6c7ccccc7c(-c7cc(-c8ccc9c(c8)[C@H]8c%10ccccc%10[C@@H]9c9ccccc98)c8ccccc8c7)c7ccccc67)cccc5c4cc23)cc1]

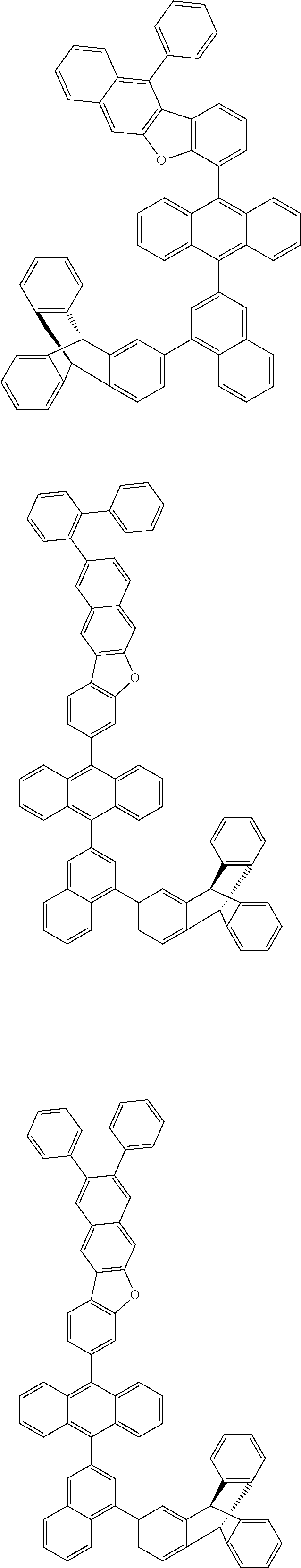 [SMILES: c1ccc(-c2c3ccccc3cc3oc4c(-c5c6ccccc6c(-c6cc(-c7ccc8c(c7)[C@H]7c9ccccc9[C@@H]8c8ccccc87)c7ccccc7c6)c6ccccc56)cccc4c23)cc1 ; c1ccc(-c2ccccc2-c2ccc3cc4oc5cc(-c6c7ccccc7c(-c7cc(-c8ccc9c(c8)[C@H]8c%10ccccc%10[C@@H]9c9ccccc98)c8ccccc8c7)c7ccccc67)ccc5c4cc3c2)cc1 ; c1ccc(-c2cc3cc4oc5cc(-c6c7ccccc7c(-c7cc(-c8ccc9c(c8)[C@H]8c%10ccccc%10[C@@H]9c9ccccc98)c8ccccc8c7)c7ccccc67)ccc5c4cc3cc2-c2ccccc2)cc1]

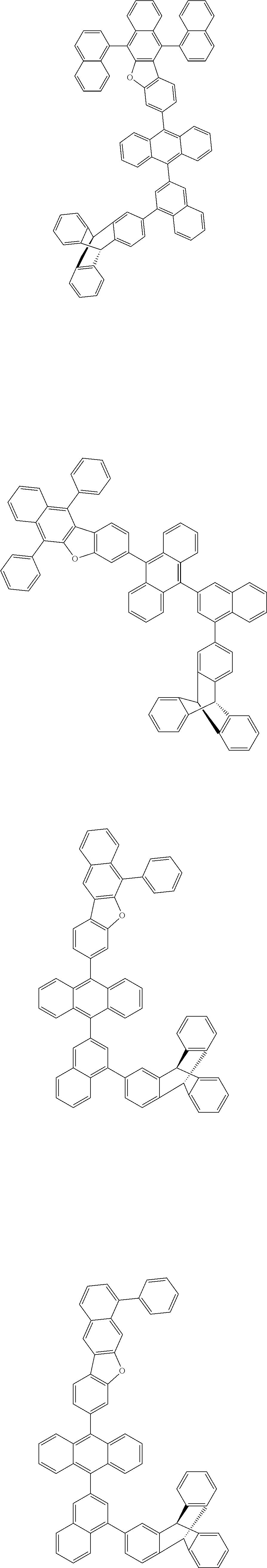

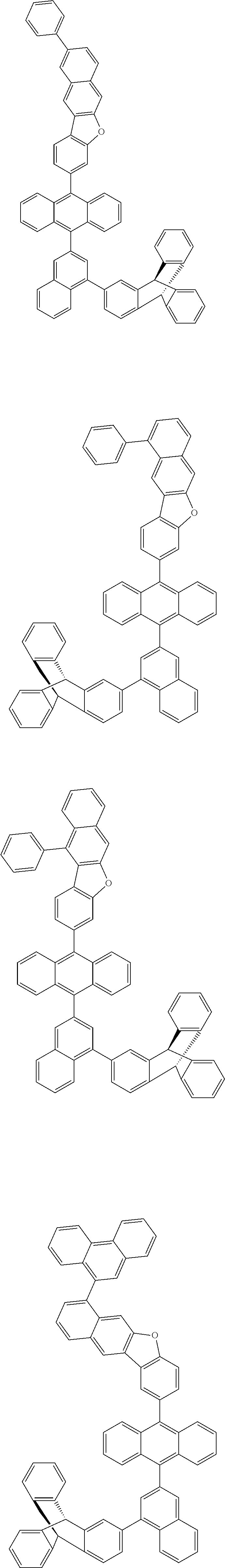

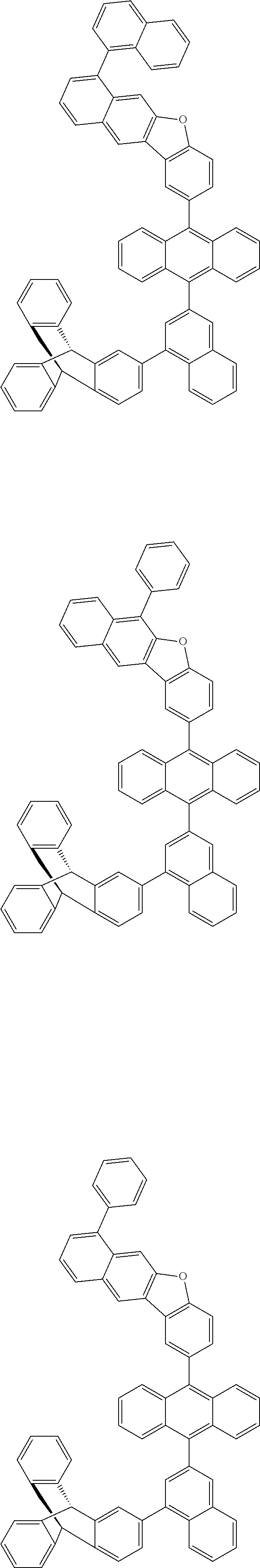 [SMILES: c1ccc2c(-c3ccc4c(c3)[C@H]3c5ccccc5[C@@H]4c4ccccc43)cc(-c3c4ccccc4c(-c4ccc5oc6cc7c(-c8cccc9ccccc89)cccc7cc6c5c4)c4ccccc34)cc2c1 ; c1ccc(-c2c3ccccc3cc3c2oc2ccc(-c4c5ccccc5c(-c5cc(-c6ccc7c(c6)[C@H]6c8ccccc8[C@@H]7c7ccccc76)c6ccccc6c5)c5ccccc45)cc23)cc1 ; c1ccc(-c2cccc3cc4c(cc23)oc2ccc(-c3c5ccccc5c(-c5cc(-c6ccc7c(c6)[C@H]6c8ccccc8[C@@H]7c7ccccc76)c6ccccc6c5)c5ccccc35)cc24)cc1]

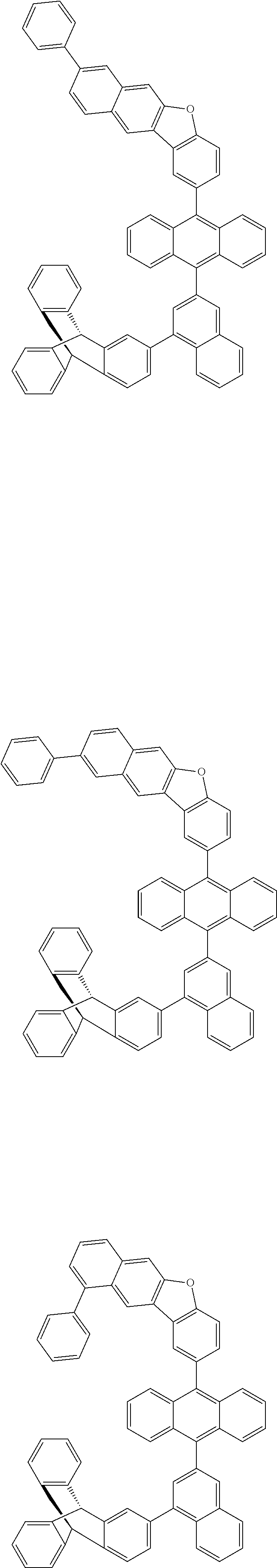 [SMILES: c1ccc(-c2ccc3cc4c(cc3c2)oc2ccc(-c3c5ccccc5c(-c5cc(-c6ccc7c(c6)[C@H]6c8ccccc8[C@@H]7c7ccccc76)c6ccccc6c5)c5ccccc35)cc24)cc1 ; c1ccc(-c2ccc3cc4oc5ccc(-c6c7ccccc7c(-c7cc(-c8ccc9c(c8)[C@H]8c%10ccccc%10[C@@H]9c9ccccc98)c8ccccc8c7)c7ccccc67)cc5c4cc3c2)cc1 ; c1ccc(-c2cccc3cc4oc5ccc(-c6c7ccccc7c(-c7cc(-c8ccc9c(c8)[C@H]8c%10ccccc%10[C@@H]9c9ccccc98)c8ccccc8c7)c7ccccc67)cc5c4cc23)cc1]

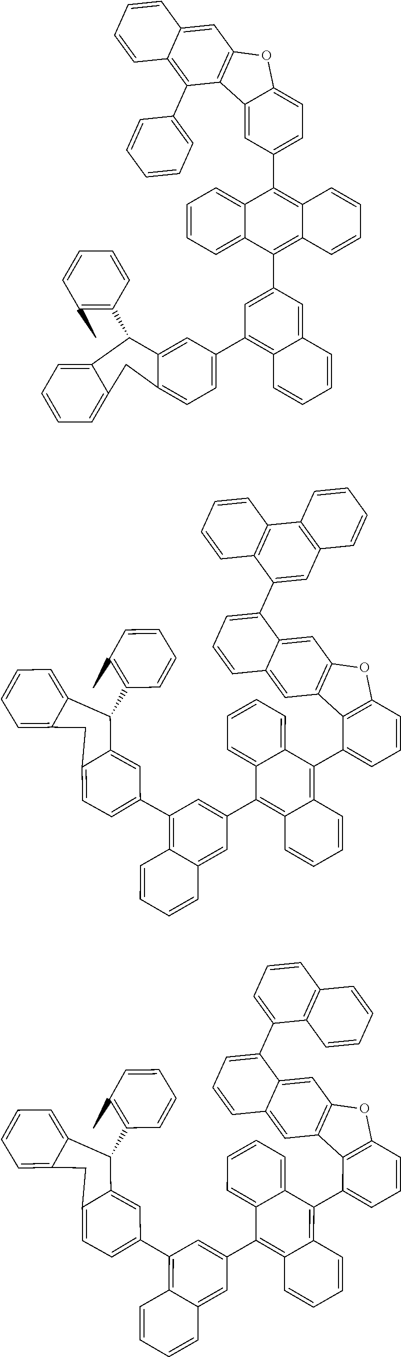

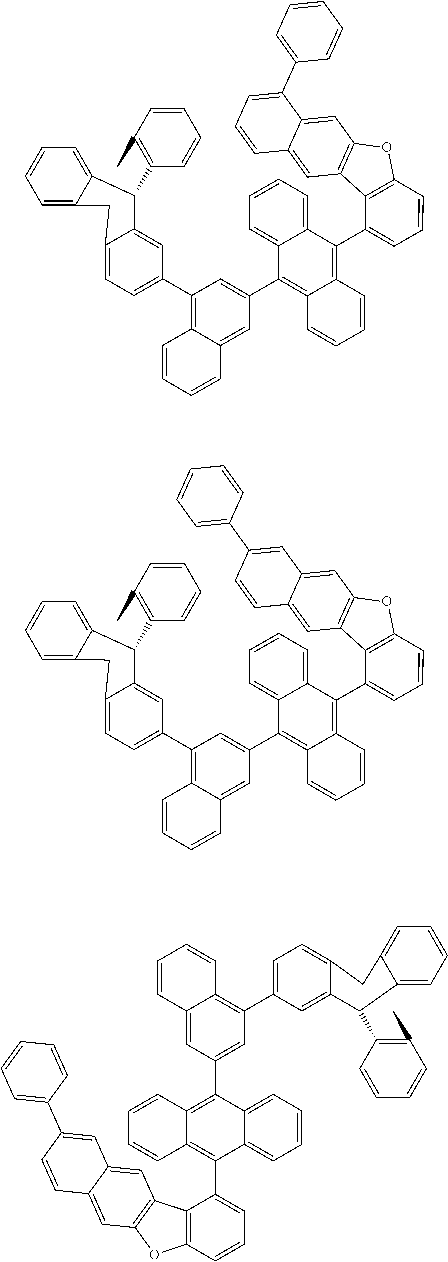

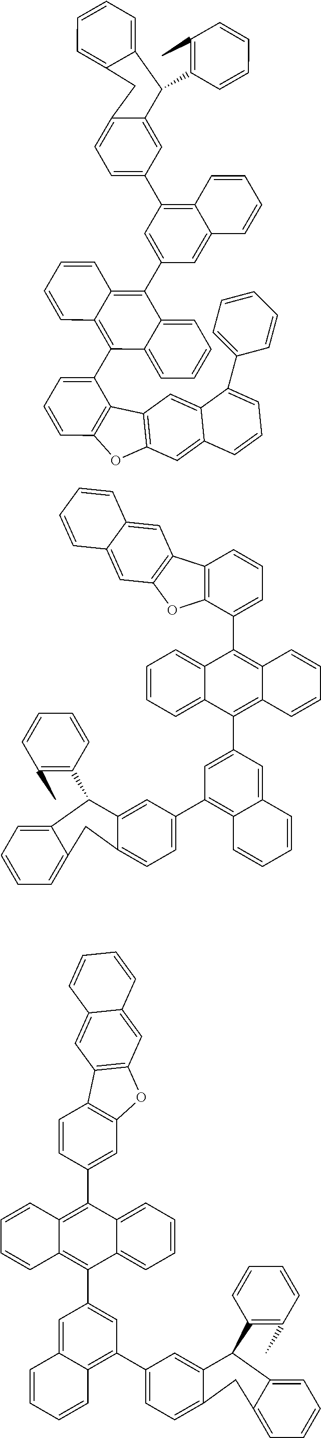

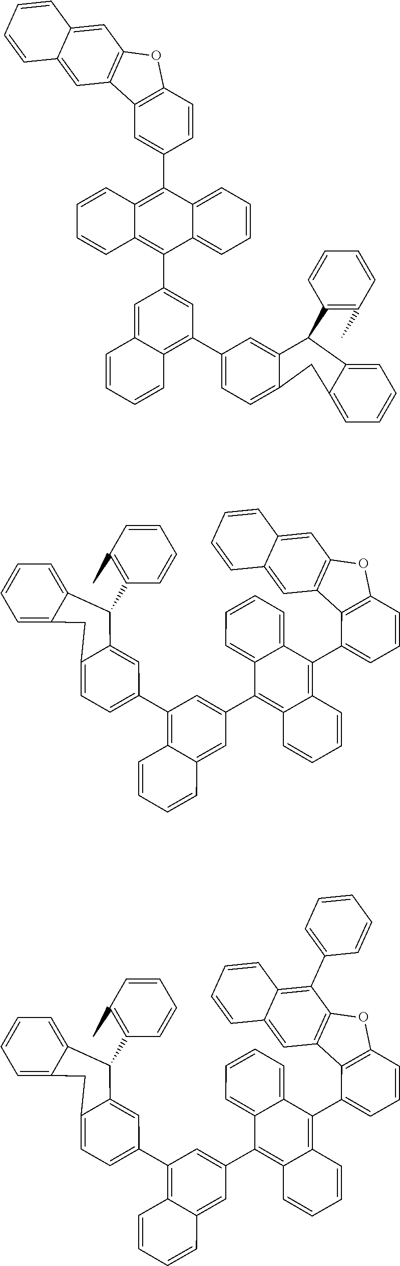

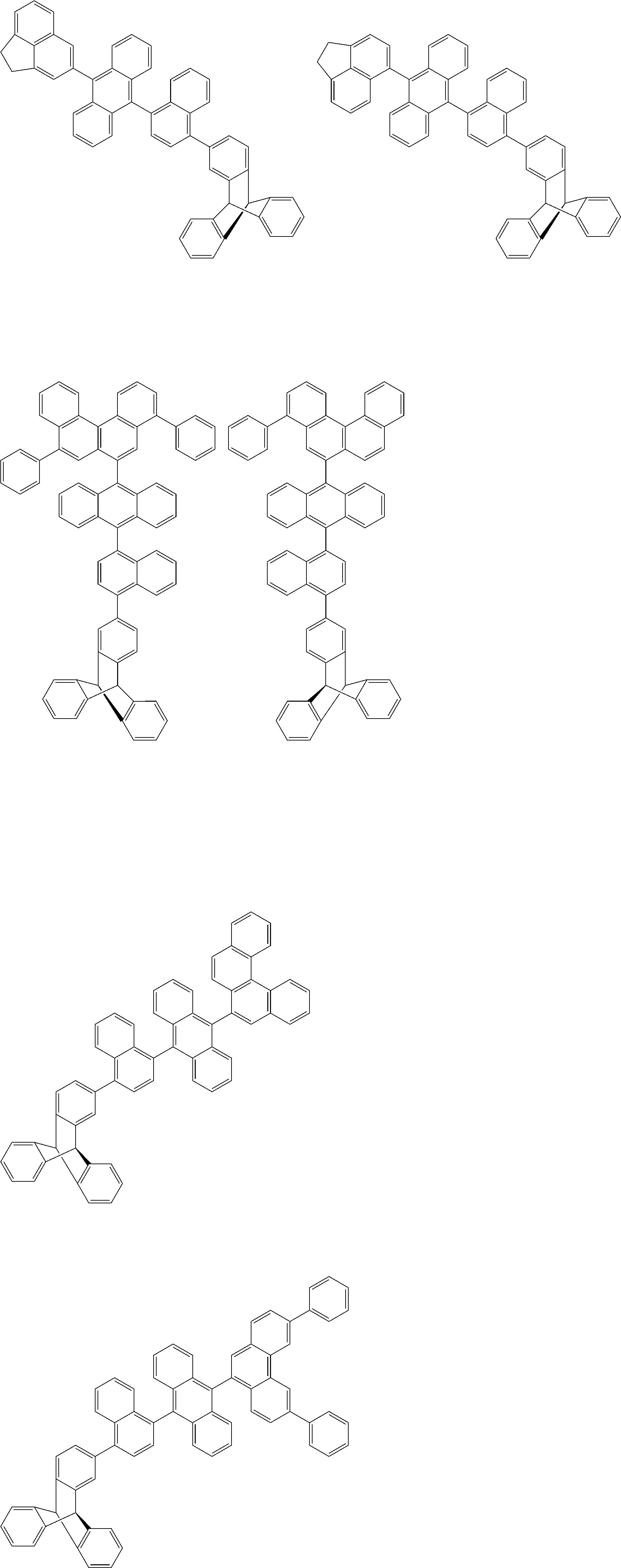

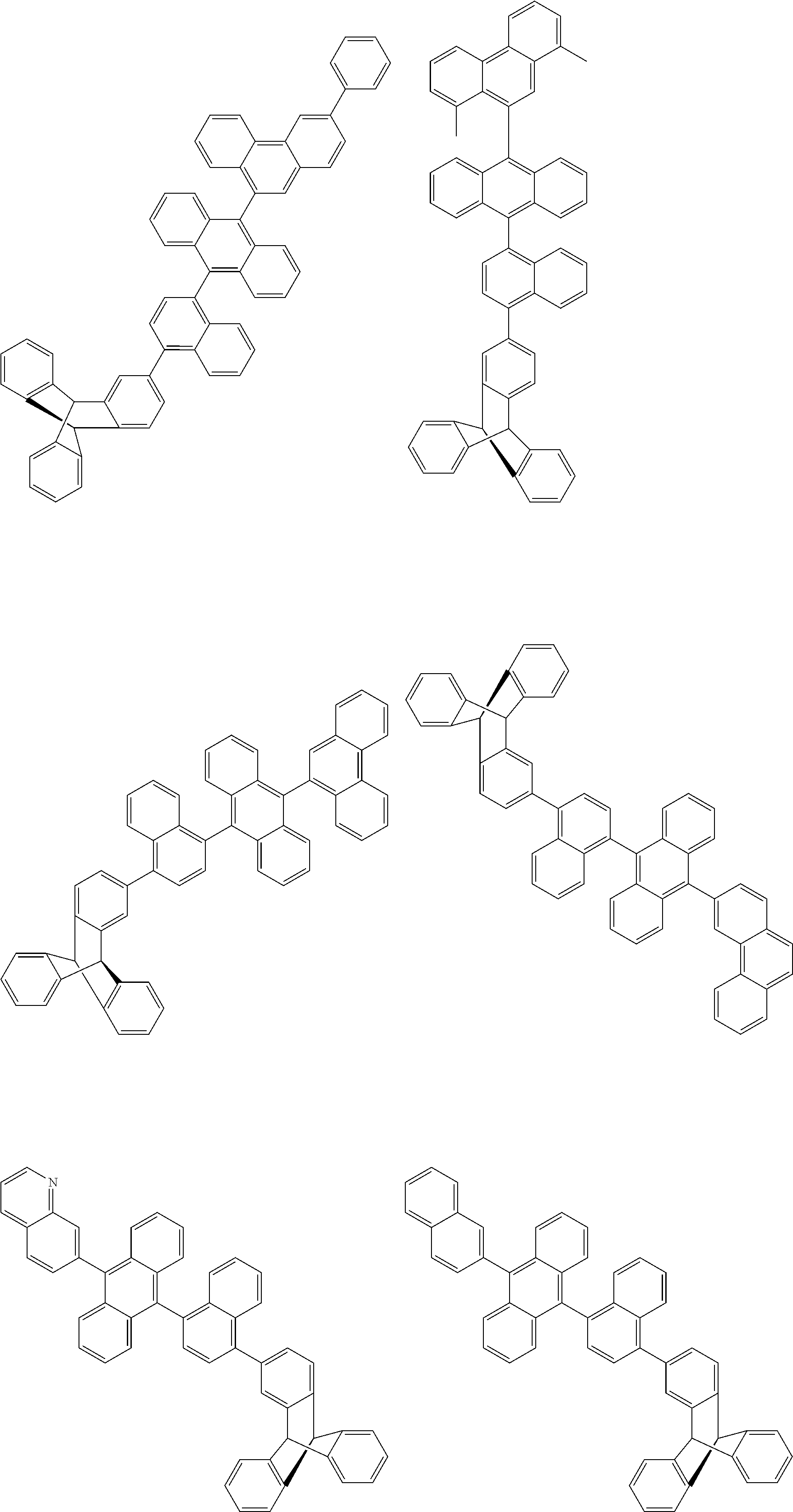

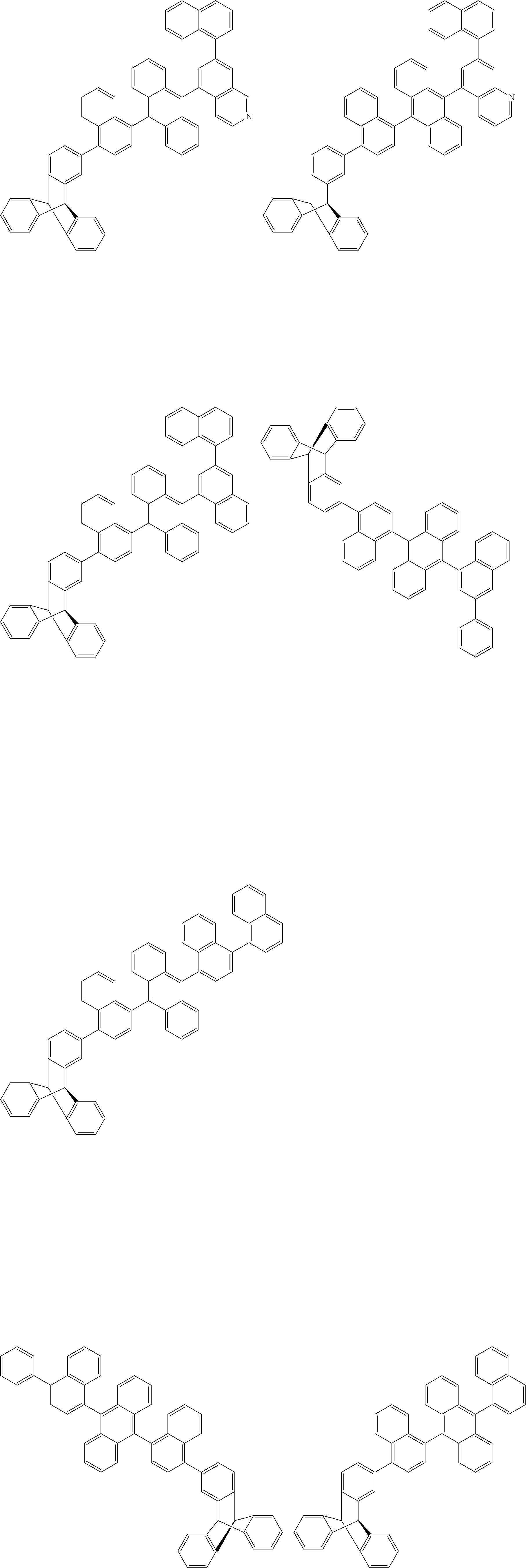

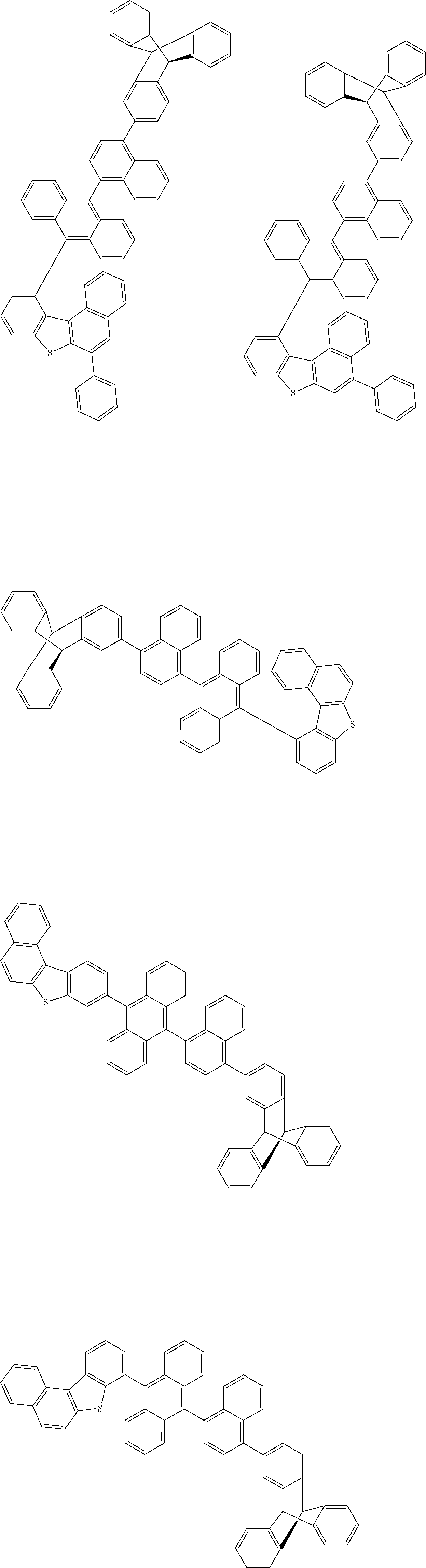

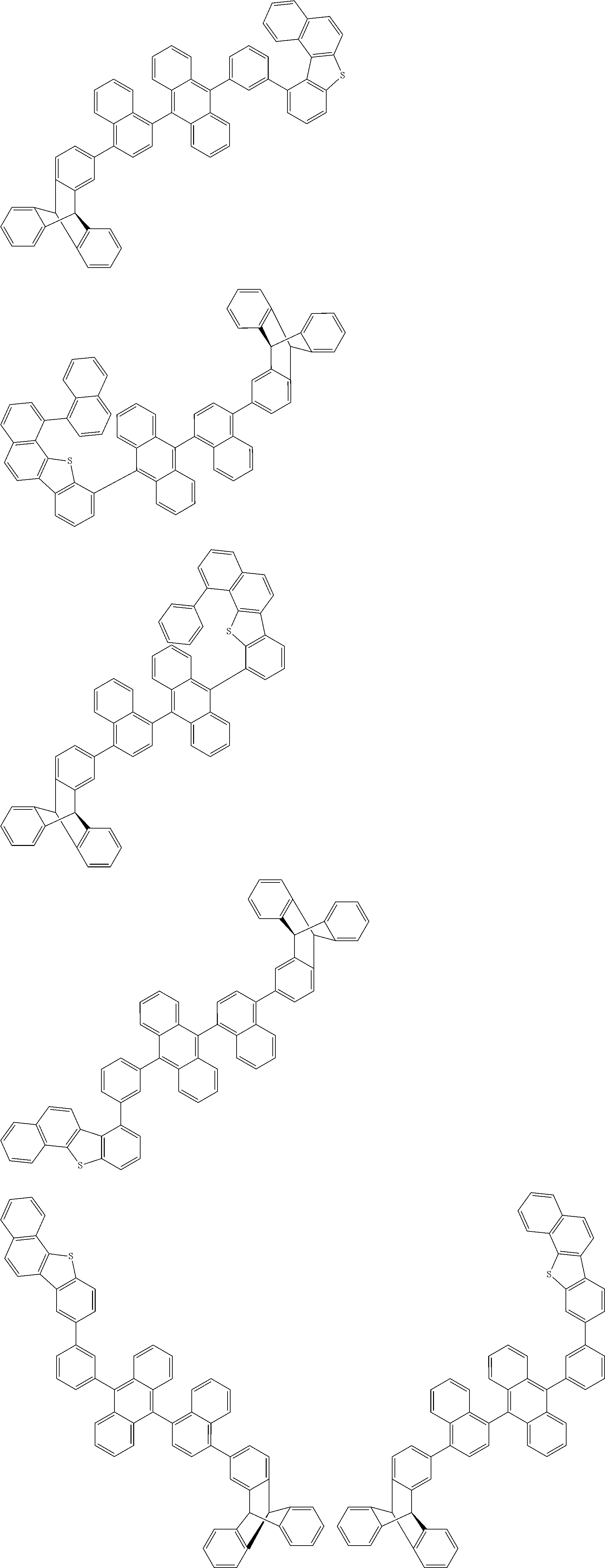

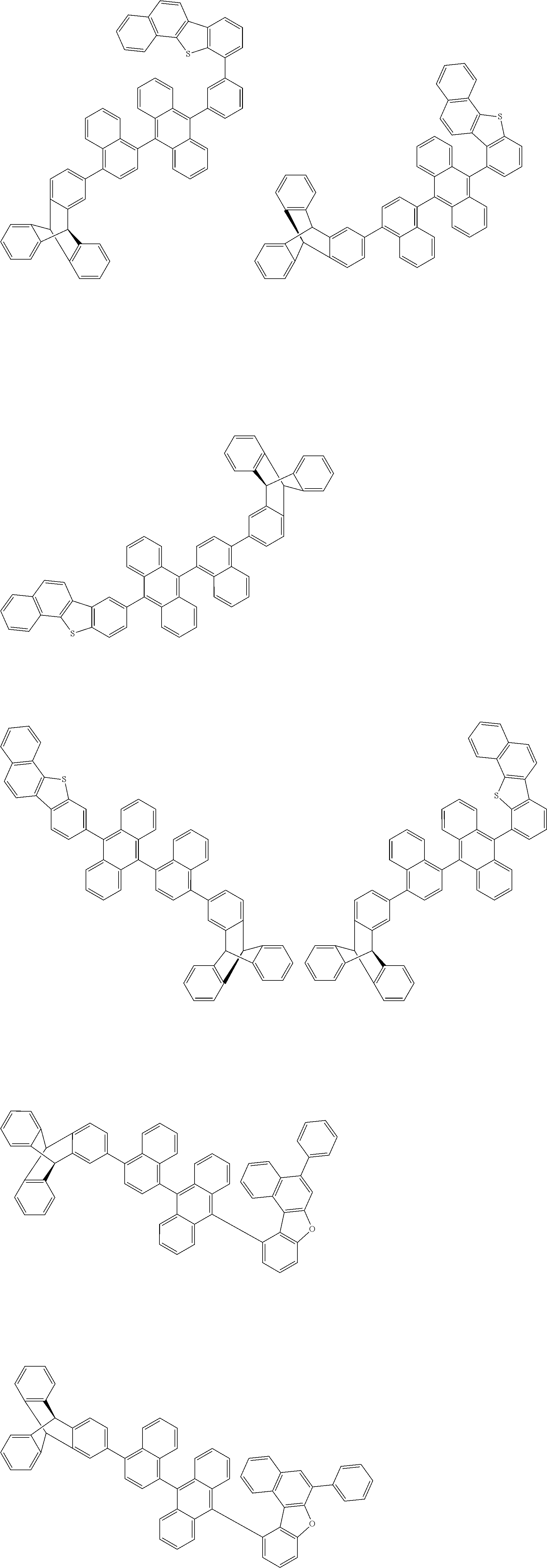

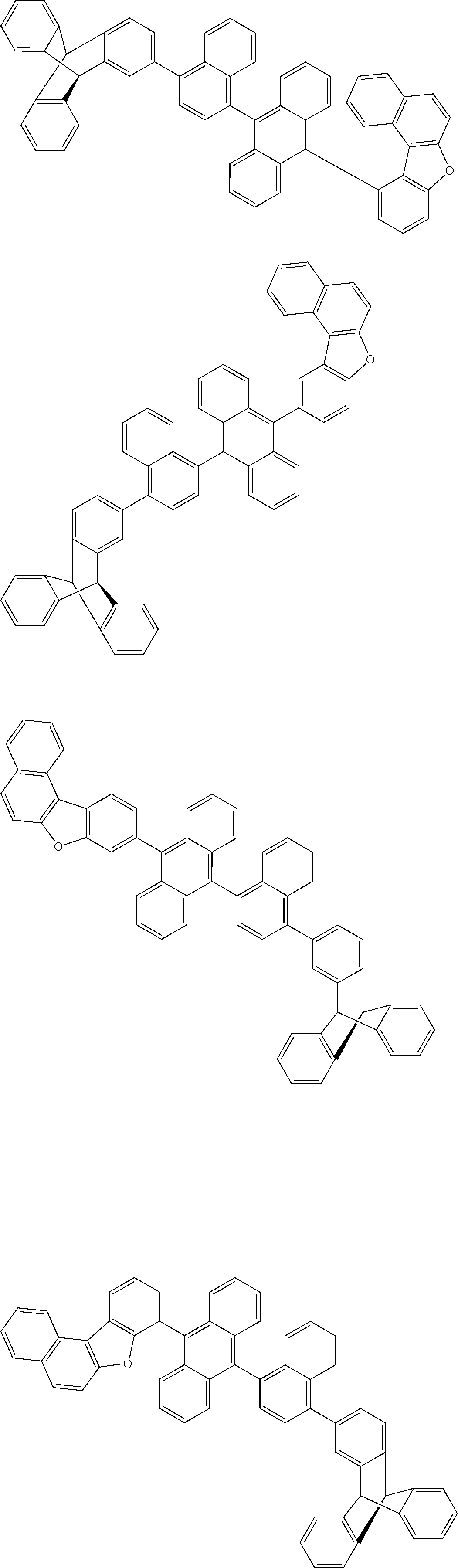

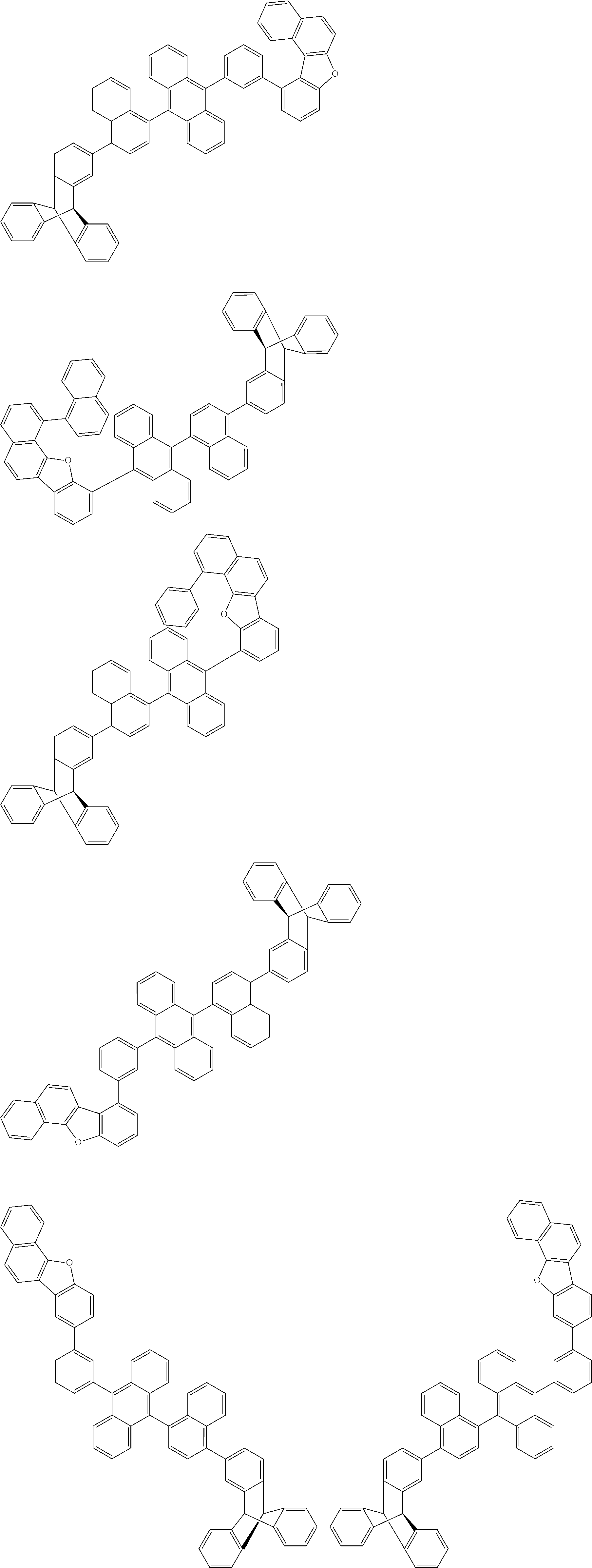

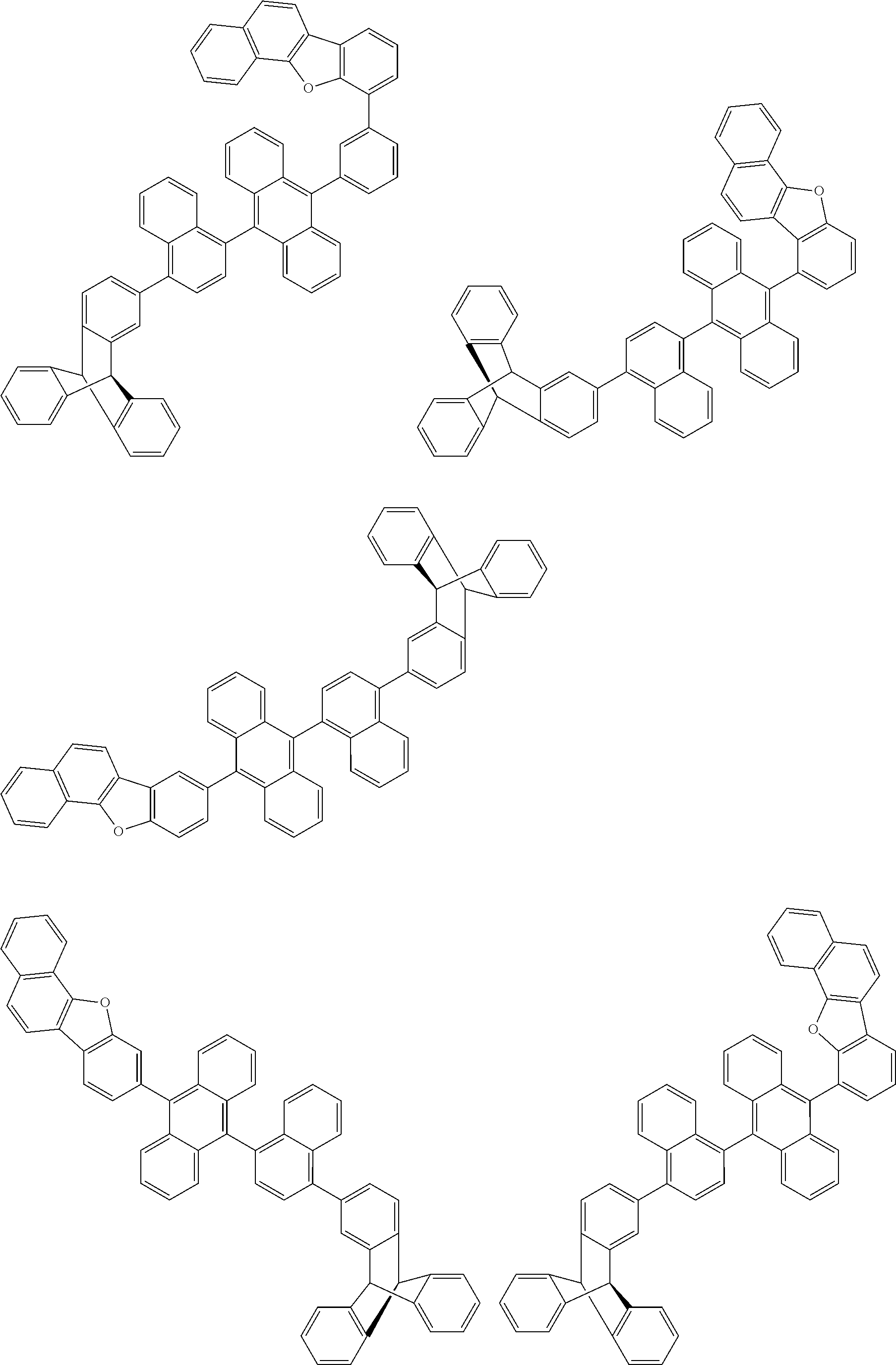

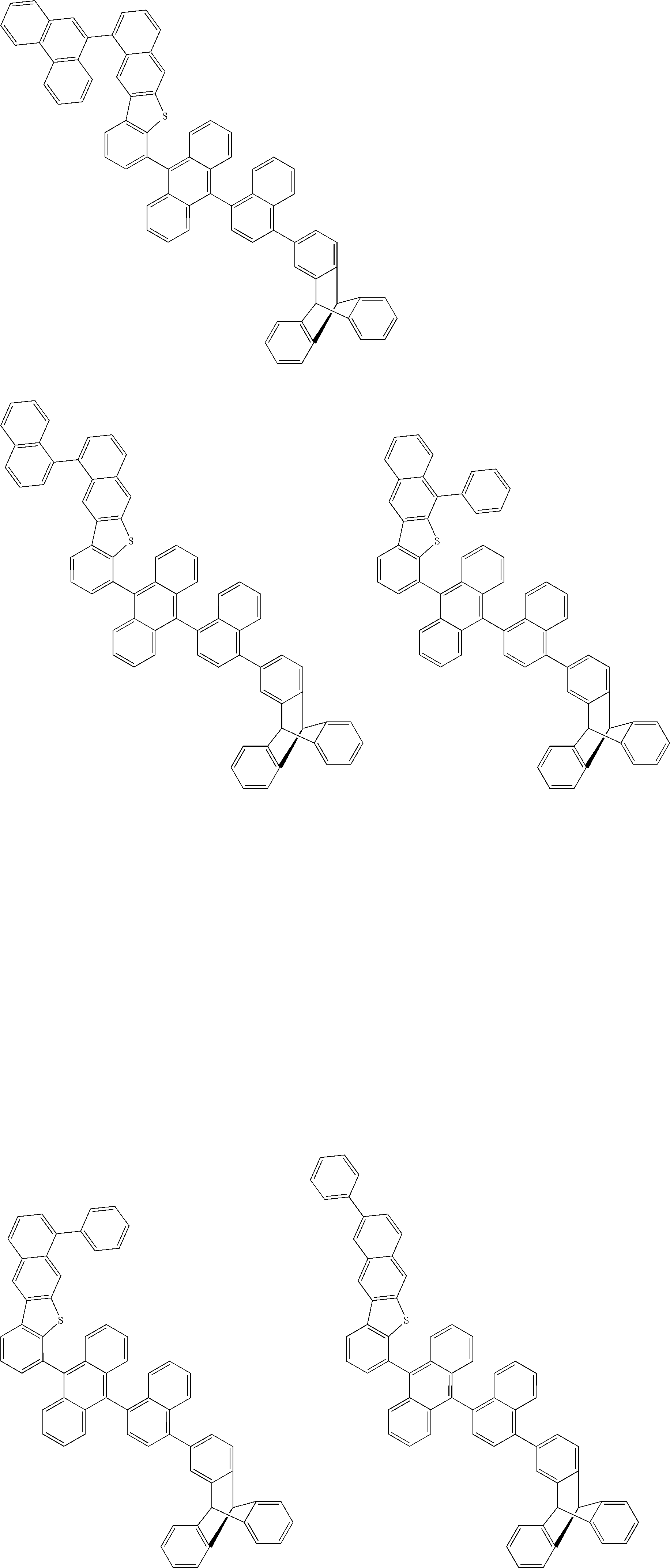

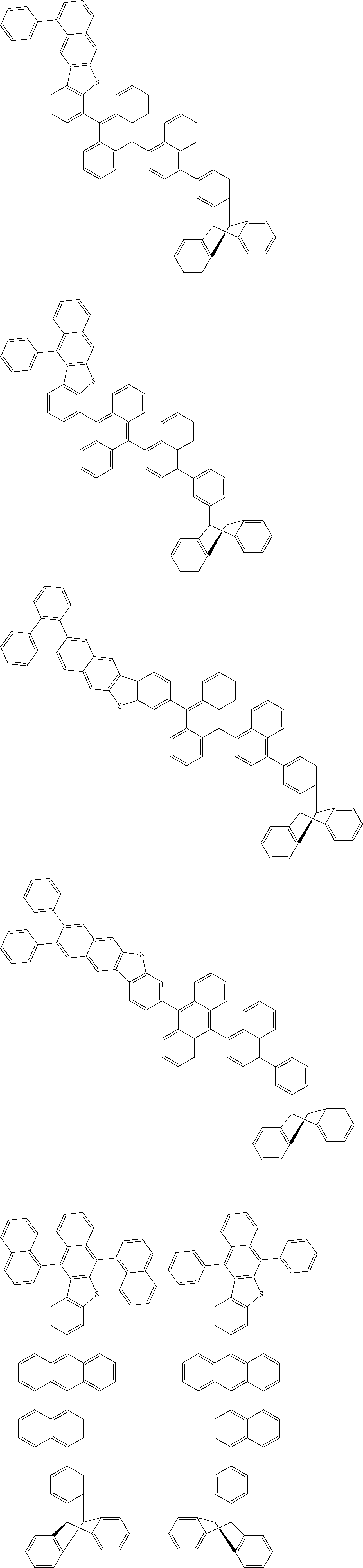

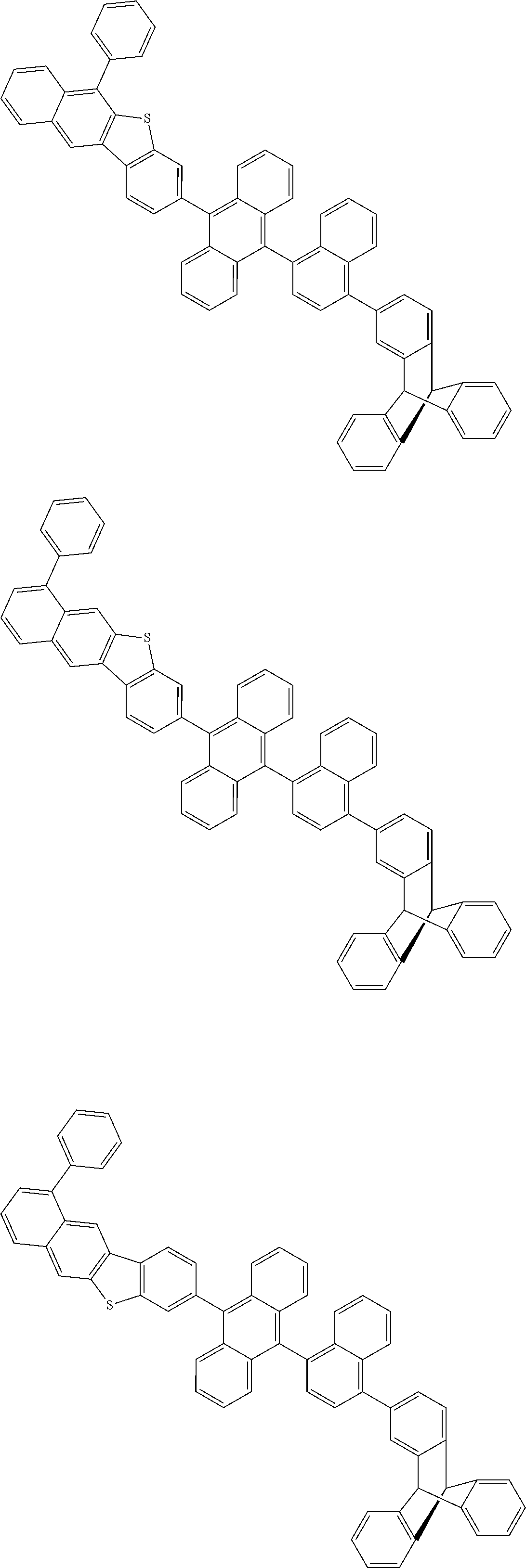

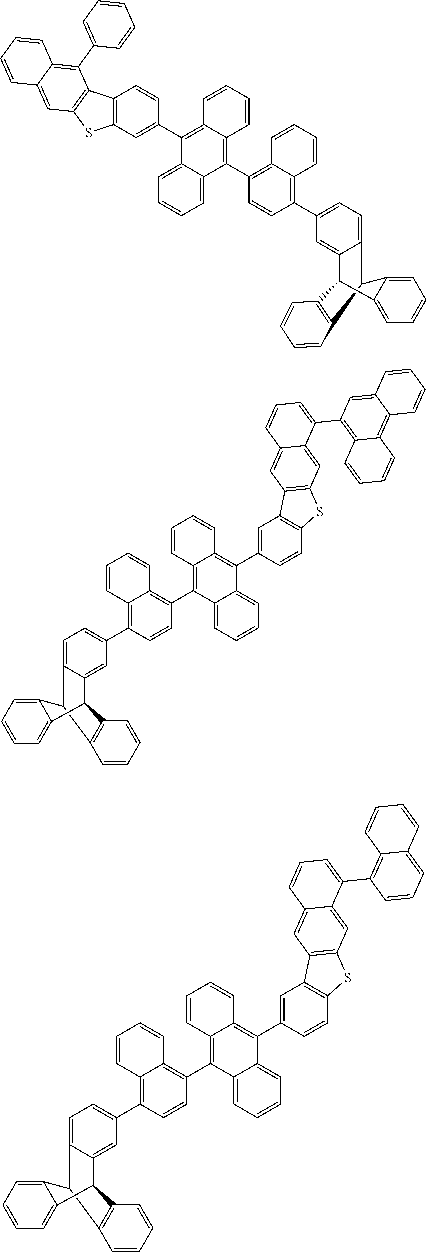

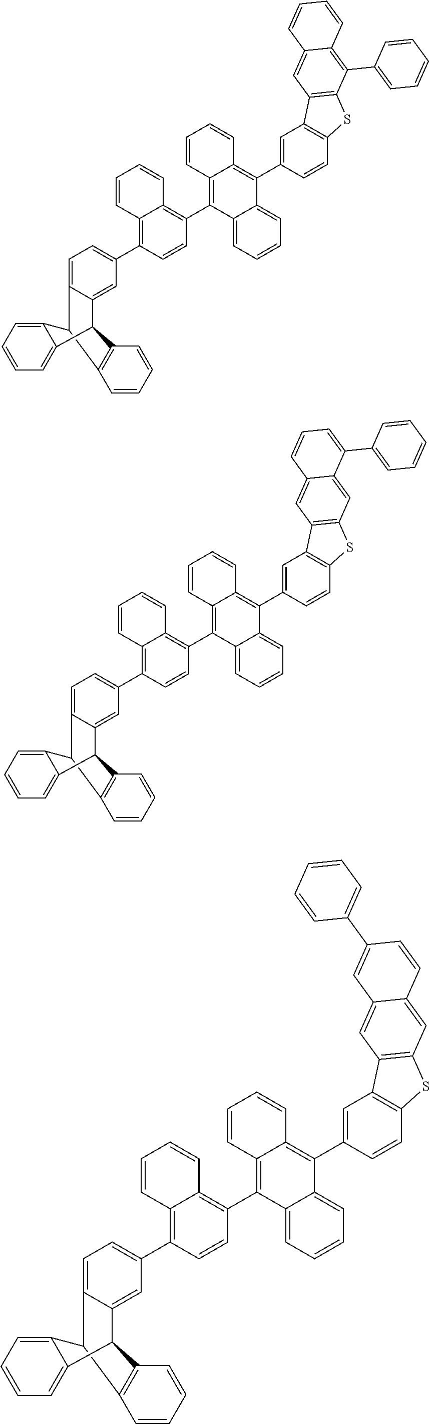

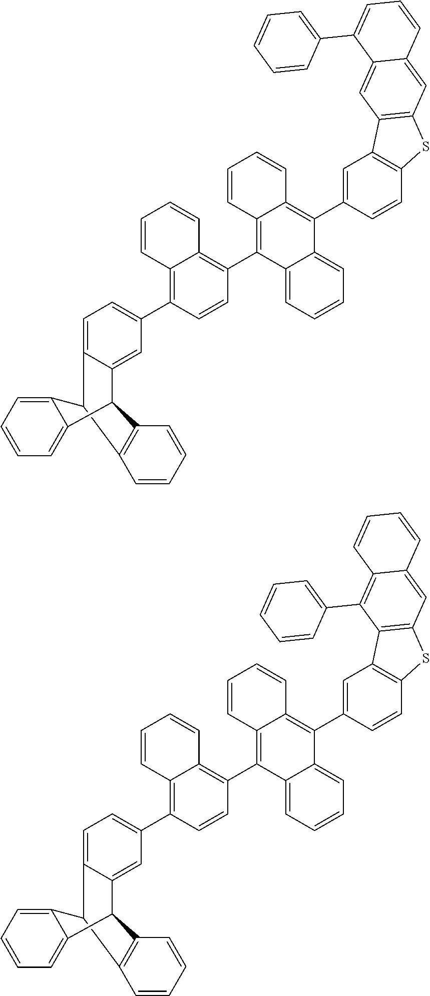

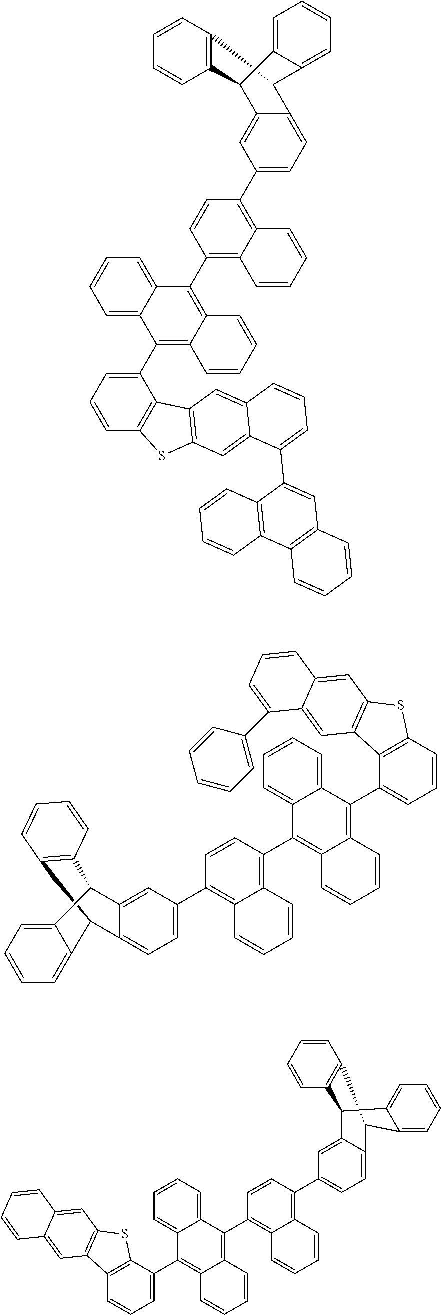 [SMILES: c1cc(-c2cc3ccccc3c3ccccc23)c2cc3sc4cccc(-c5c6ccccc6c(-c6ccc(-c7ccc8c(c7)[C@H]7c9ccccc9[C@@H]8c8ccccc87)c7ccccc67)c6ccccc56)c4c3cc2c1 ; c1ccc(-c2cccc3cc4sc5cccc(-c6c7ccccc7c(-c7ccc(-c8ccc9c(c8)[C@H]8c%10ccccc%10[C@@H]9c9ccccc98)c8ccccc78)c7ccccc67)c5c4cc23)cc1 ; c1ccc2cc3c(cc2c1)sc1c(-c2c4ccccc4c(-c4ccc(-c5ccc6c(c5)[C@H]5c7ccccc7[C@@H]6c6ccccc65)c5ccccc45)c4ccccc24)cccc13]

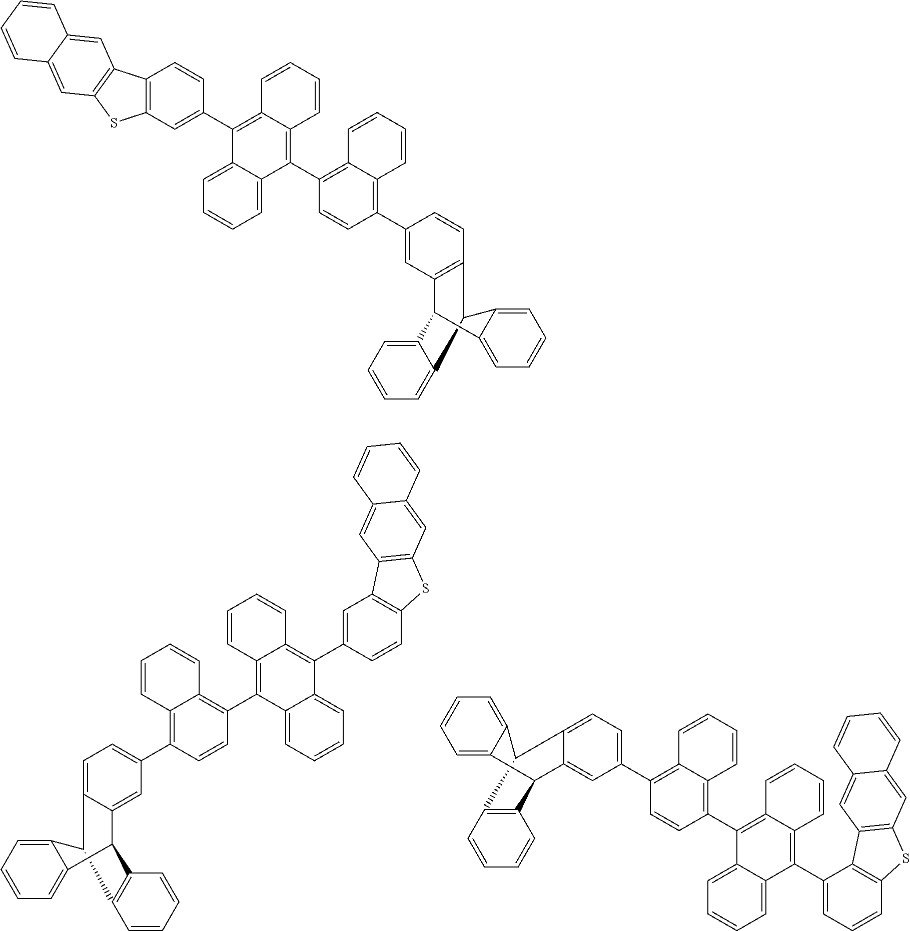 [SMILES: c1ccc2cc3c(cc2c1)sc1cc(-c2c4ccccc4c(-c4ccc(-c5ccc6c(c5)[C@H]5c7ccccc7[C@@H]6c6ccccc65)c5ccccc45)c4ccccc24)ccc13 ; c1ccc2cc3c(cc2c1)sc1ccc(-c2c4ccccc4c(-c4ccc(-c5ccc6c(c5)[C@H]5c7ccccc7[C@@H]6c6ccccc65)c5ccccc45)c4ccccc24)cc13 ; c1ccc2cc3c(cc2c1)sc1cccc(-c2c4ccccc4c(-c4ccc(-c5ccc6c(c5)[C@H]5c7ccccc7[C@@H]6c6ccccc65)c5ccccc45)c4ccccc24)c13]

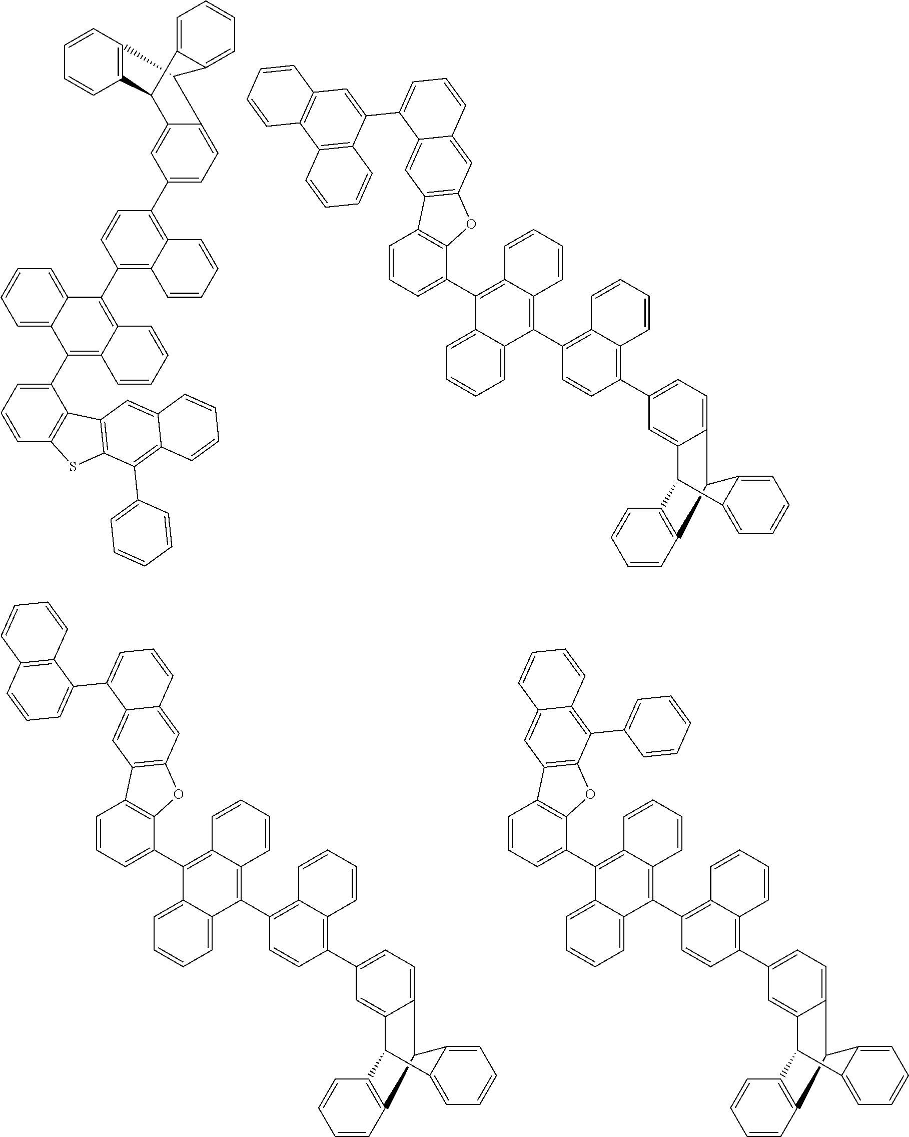

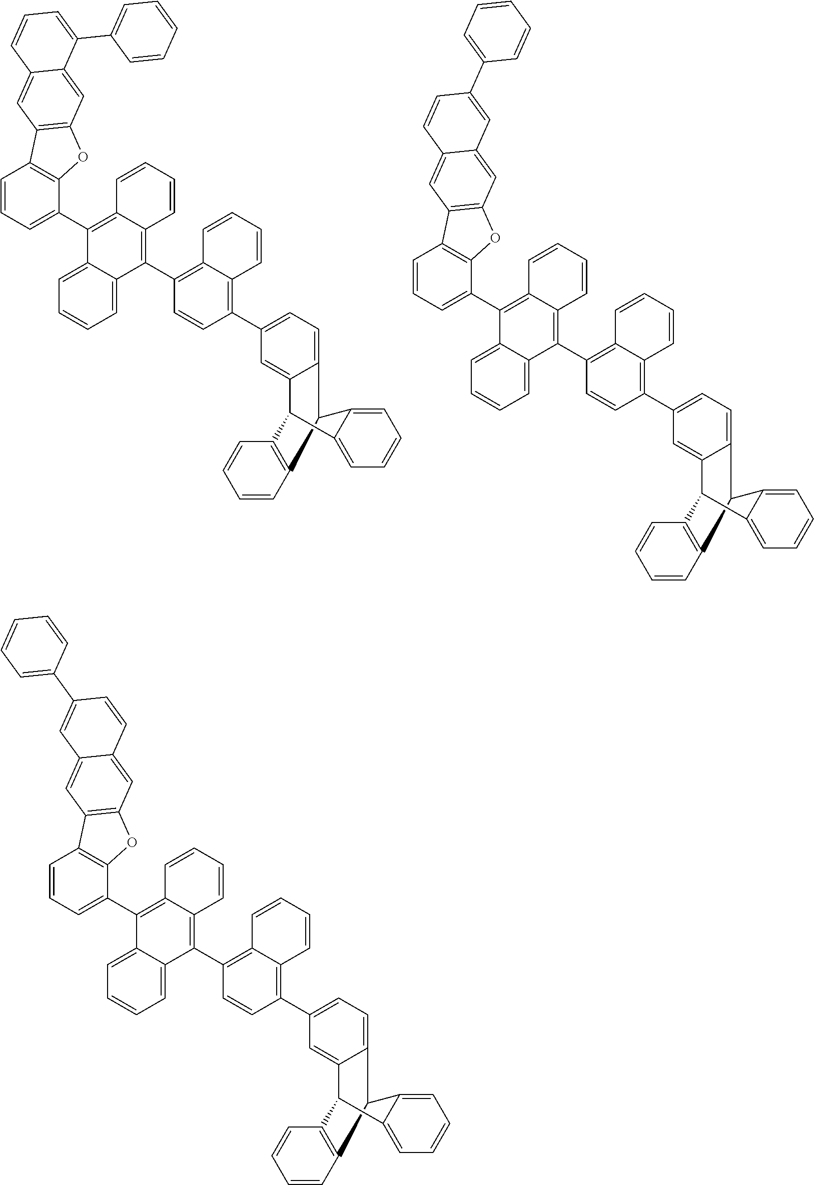 [SMILES: c1ccc(-c2cccc3cc4c(cc23)oc2c(-c3c5ccccc5c(-c5ccc(-c6ccc7c(c6)[C@H]6c8ccccc8[C@@H]7c7ccccc76)c6ccccc56)c5ccccc35)cccc24)cc1 ; c1ccc(-c2ccc3cc4c(cc3c2)oc2c(-c3c5ccccc5c(-c5ccc(-c6ccc7c(c6)[C@H]6c8ccccc8[C@@H]7c7ccccc76)c6ccccc56)c5ccccc35)cccc24)cc1 ; c1ccc(-c2ccc3cc4oc5c(-c6c7ccccc7c(-c7ccc(-c8ccc9c(c8)[C@H]8c%10ccccc%10[C@@H]9c9ccccc98)c8ccccc78)c7ccccc67)cccc5c4cc3c2)cc1]

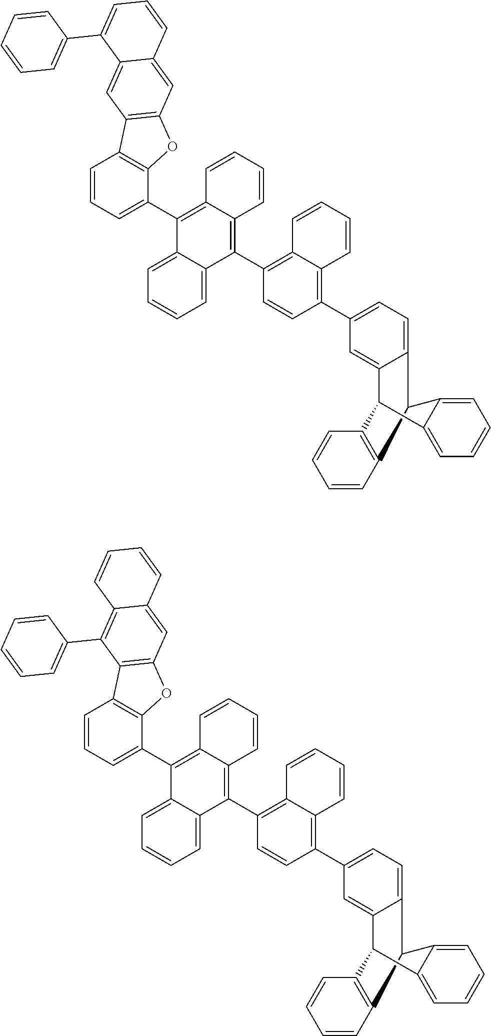 [SMILES: c1ccc(-c2cccc3cc4oc5c(-c6c7ccccc7c(-c7ccc(-c8ccc9c(c8)[C@H]8c%10ccccc%10[C@@H]9c9ccccc98)c8ccccc78)c7ccccc67)cccc5c4cc23)cc1 ; c1ccc(-c2c3ccccc3cc3oc4c(-c5c6ccccc6c(-c6ccc(-c7ccc8c(c7)[C@H]7c9ccccc9[C@@H]8c8ccccc87)c7ccccc67)c6ccccc56)cccc4c23)cc1]

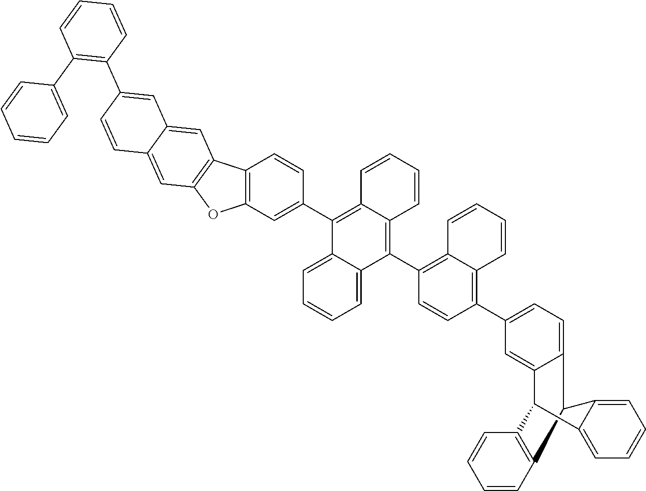 [SMILES: c1ccc(-c2ccccc2-c2ccc3cc4oc5cc(-c6c7ccccc7c(-c7ccc(-c8ccc9c(c8)[C@H]8c%10ccccc%10[C@@H]9c9ccccc98)c8ccccc78)c7ccccc67)ccc5c4cc3c2)cc1]

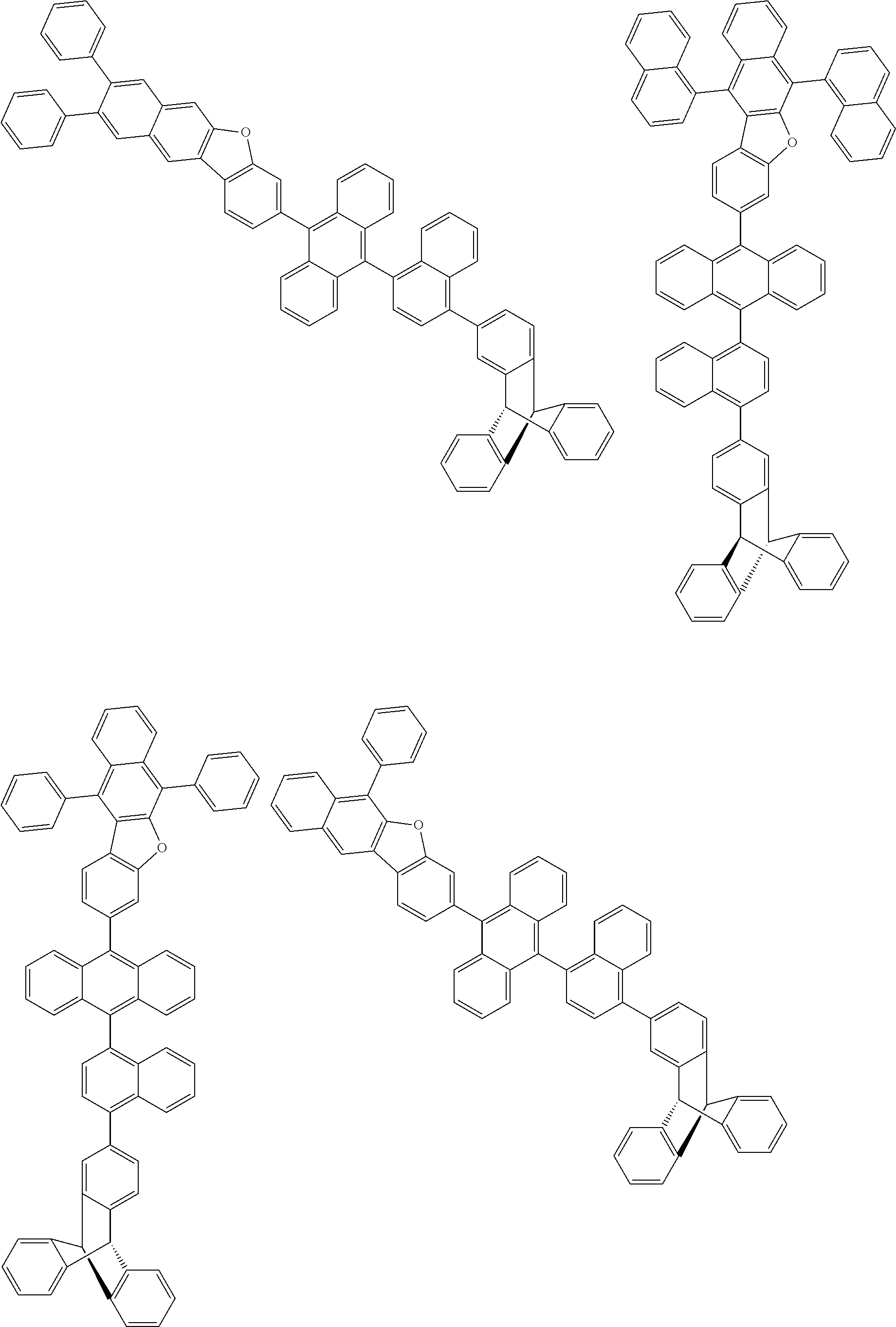

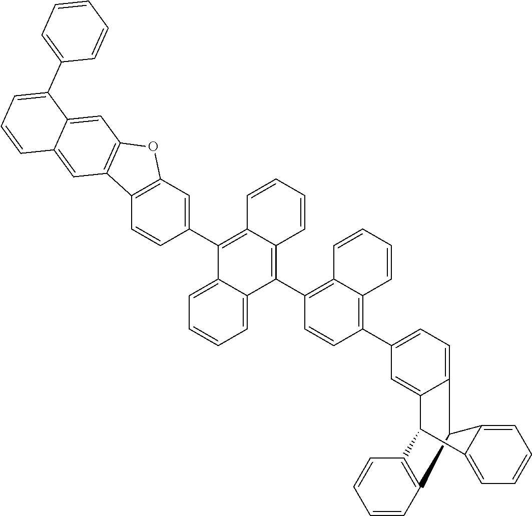 [SMILES: c1ccc(-c2cccc3cc4c(cc23)oc2cc(-c3c5ccccc5c(-c5ccc(-c6ccc7c(c6)[C@H]6c8ccccc8[C@@H]7c7ccccc76)c6ccccc56)c5ccccc35)ccc24)cc1]

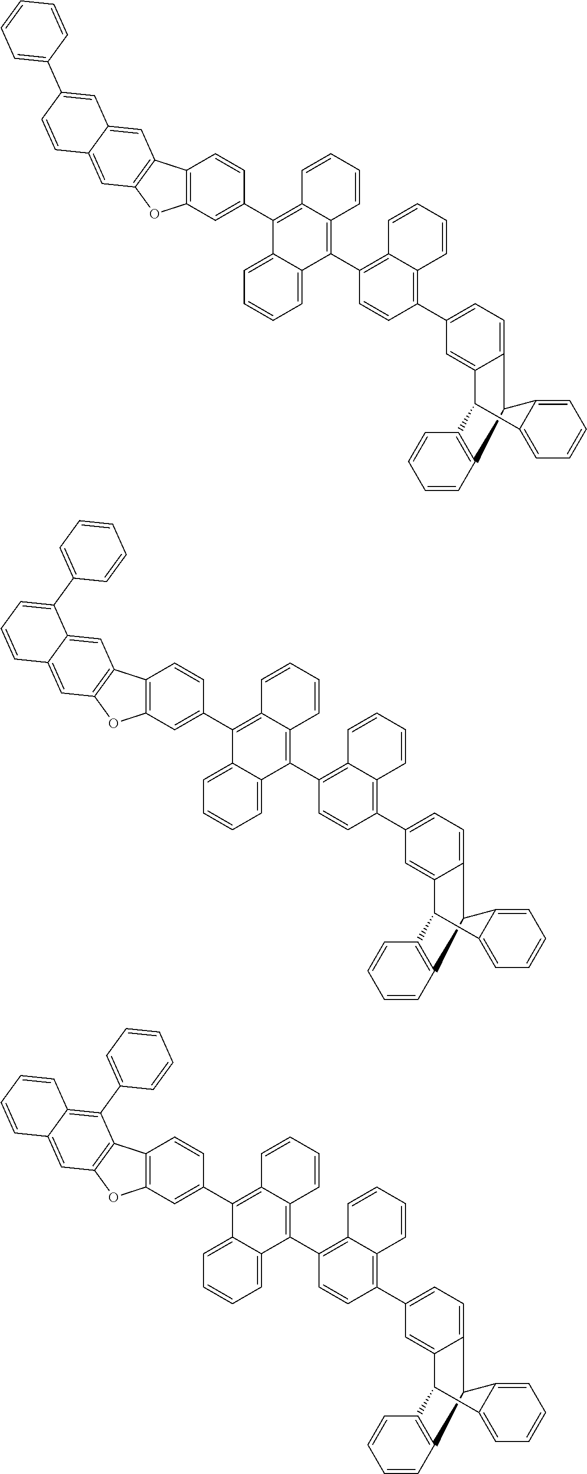 [SMILES: c1ccc(-c2ccc3cc4oc5cc(-c6c7ccccc7c(-c7ccc(-c8ccc9c(c8)[C@H]8c%10ccccc%10[C@@H]9c9ccccc98)c8ccccc78)c7ccccc67)ccc5c4cc3c2)cc1 ; c1ccc(-c2cccc3cc4oc5cc(-c6c7ccccc7c(-c7ccc(-c8ccc9c(c8)[C@H]8c%10ccccc%10[C@@H]9c9ccccc98)c8ccccc78)c7ccccc67)ccc5c4cc23)cc1 ; c1ccc(-c2c3ccccc3cc3oc4cc(-c5c6ccccc6c(-c6ccc(-c7ccc8c(c7)[C@H]7c9ccccc9[C@@H]8c8ccccc87)c7ccccc67)c6ccccc56)ccc4c23)cc1]

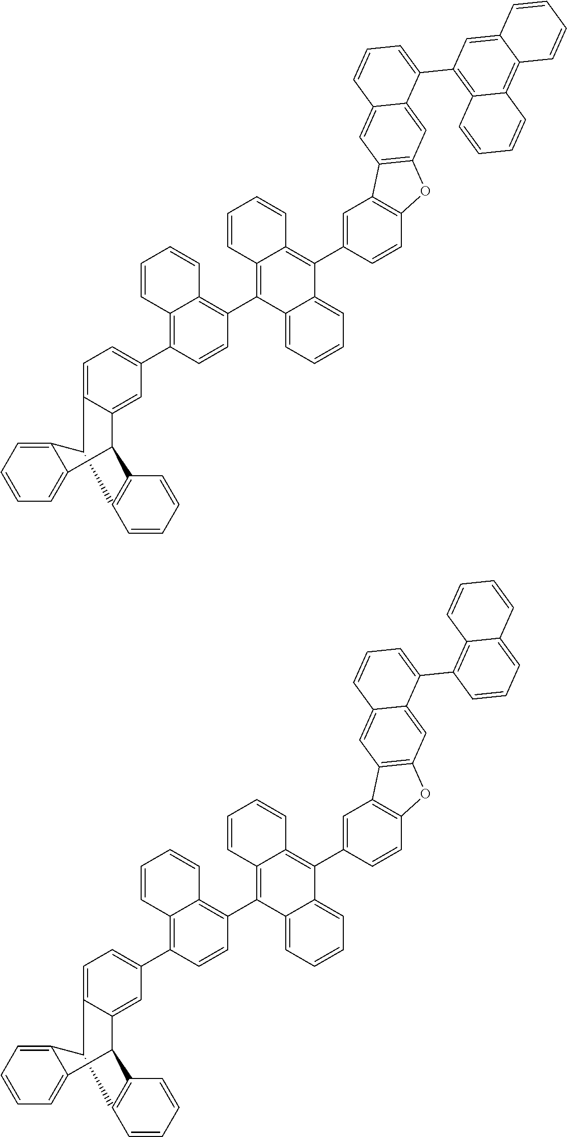 [SMILES: c1cc(-c2cc3ccccc3c3ccccc23)c2cc3oc4ccc(-c5c6ccccc6c(-c6ccc(-c7ccc8c(c7)[C@H]7c9ccccc9[C@@H]8c8ccccc87)c7ccccc67)c6ccccc56)cc4c3cc2c1 ; c1ccc2c(-c3cccc4cc5c(cc34)oc3ccc(-c4c6ccccc6c(-c6ccc(-c7ccc8c(c7)[C@H]7c9ccccc9[C@@H]8c8ccccc87)c7ccccc67)c6ccccc46)cc35)cccc2c1]

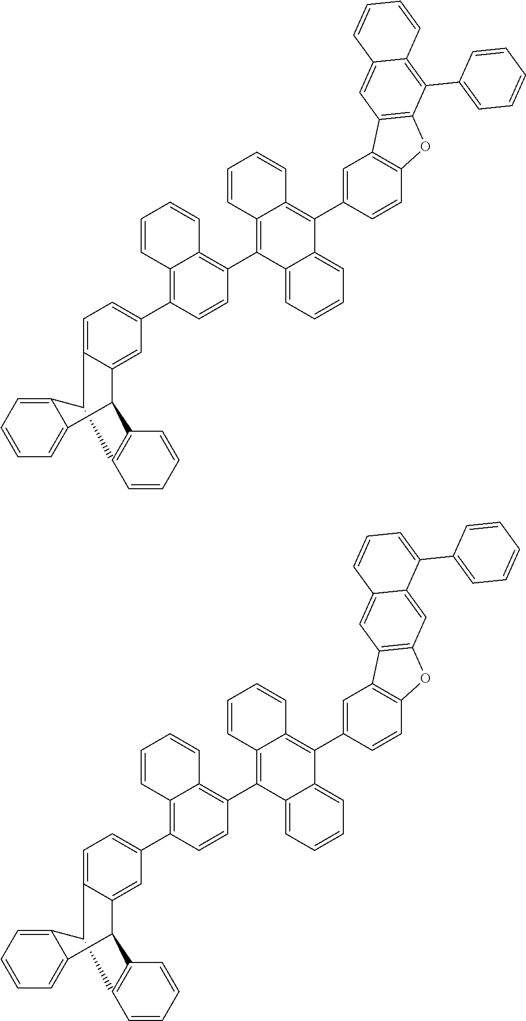 [SMILES: c1ccc(-c2c3ccccc3cc3c2oc2ccc(-c4c5ccccc5c(-c5ccc(-c6ccc7c(c6)[C@H]6c8ccccc8[C@@H]7c7ccccc76)c6ccccc56)c5ccccc45)cc23)cc1 ; c1ccc(-c2cccc3cc4c(cc23)oc2ccc(-c3c5ccccc5c(-c5ccc(-c6ccc7c(c6)[C@H]6c8ccccc8[C@@H]7c7ccccc76)c6ccccc56)c5ccccc35)cc24)cc1]

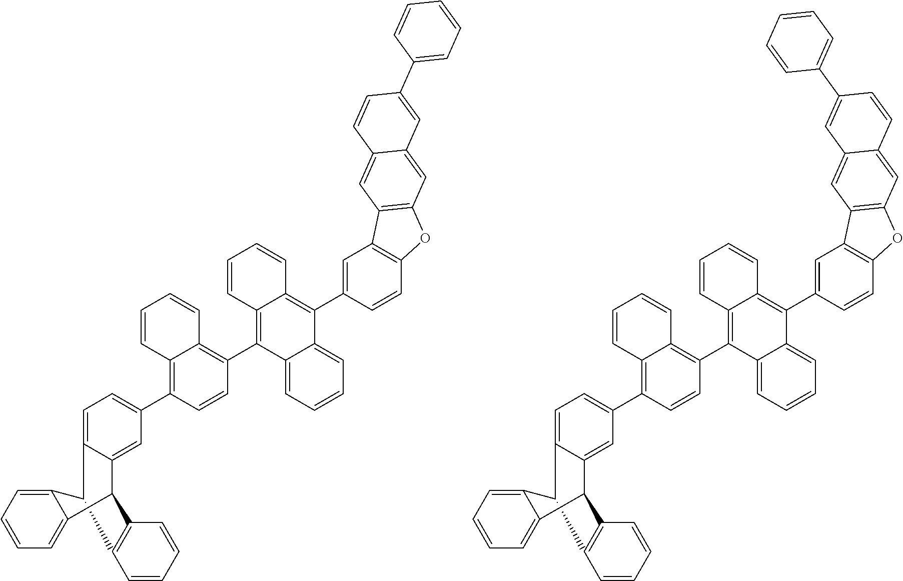 [SMILES: c1ccc(-c2ccc3cc4c(cc3c2)oc2ccc(-c3c5ccccc5c(-c5ccc(-c6ccc7c(c6)[C@H]6c8ccccc8[C@@H]7c7ccccc76)c6ccccc56)c5ccccc35)cc24)cc1 ; c1ccc(-c2ccc3cc4oc5ccc(-c6c7ccccc7c(-c7ccc(-c8ccc9c(c8)[C@H]8c%10ccccc%10[C@@H]9c9ccccc98)c8ccccc78)c7ccccc67)cc5c4cc3c2)cc1]

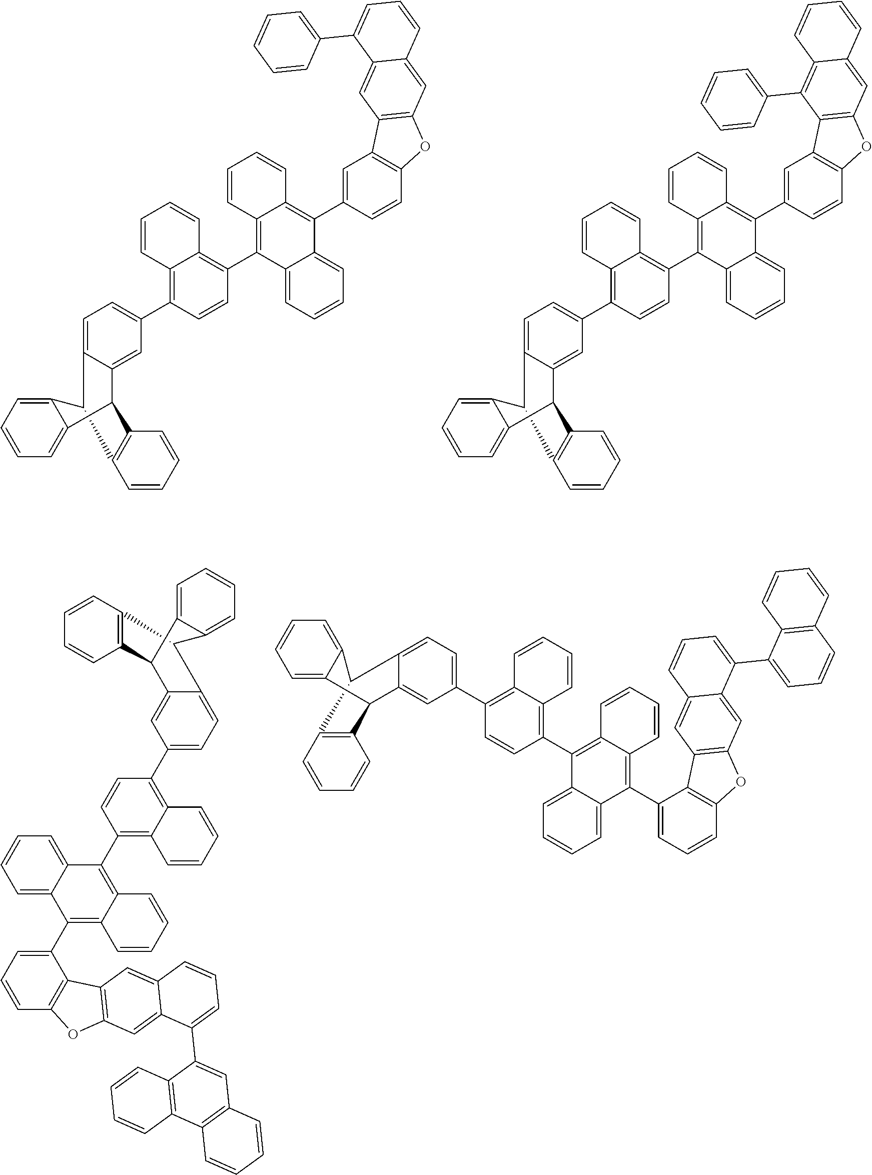

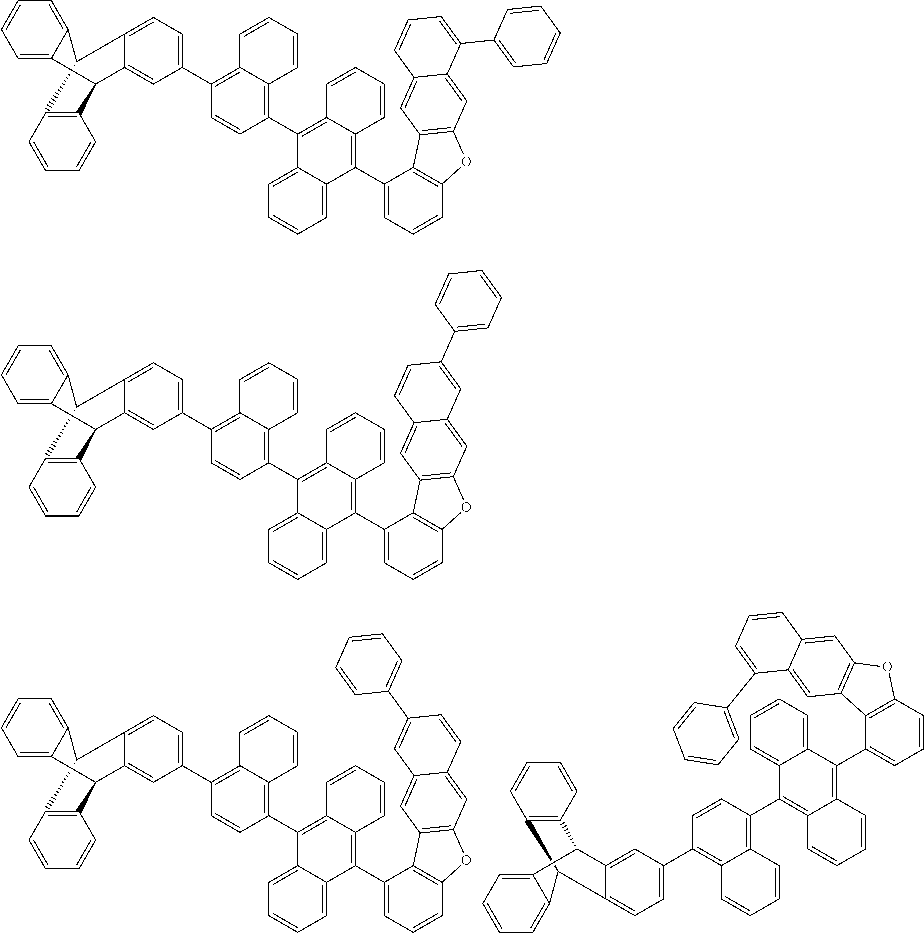

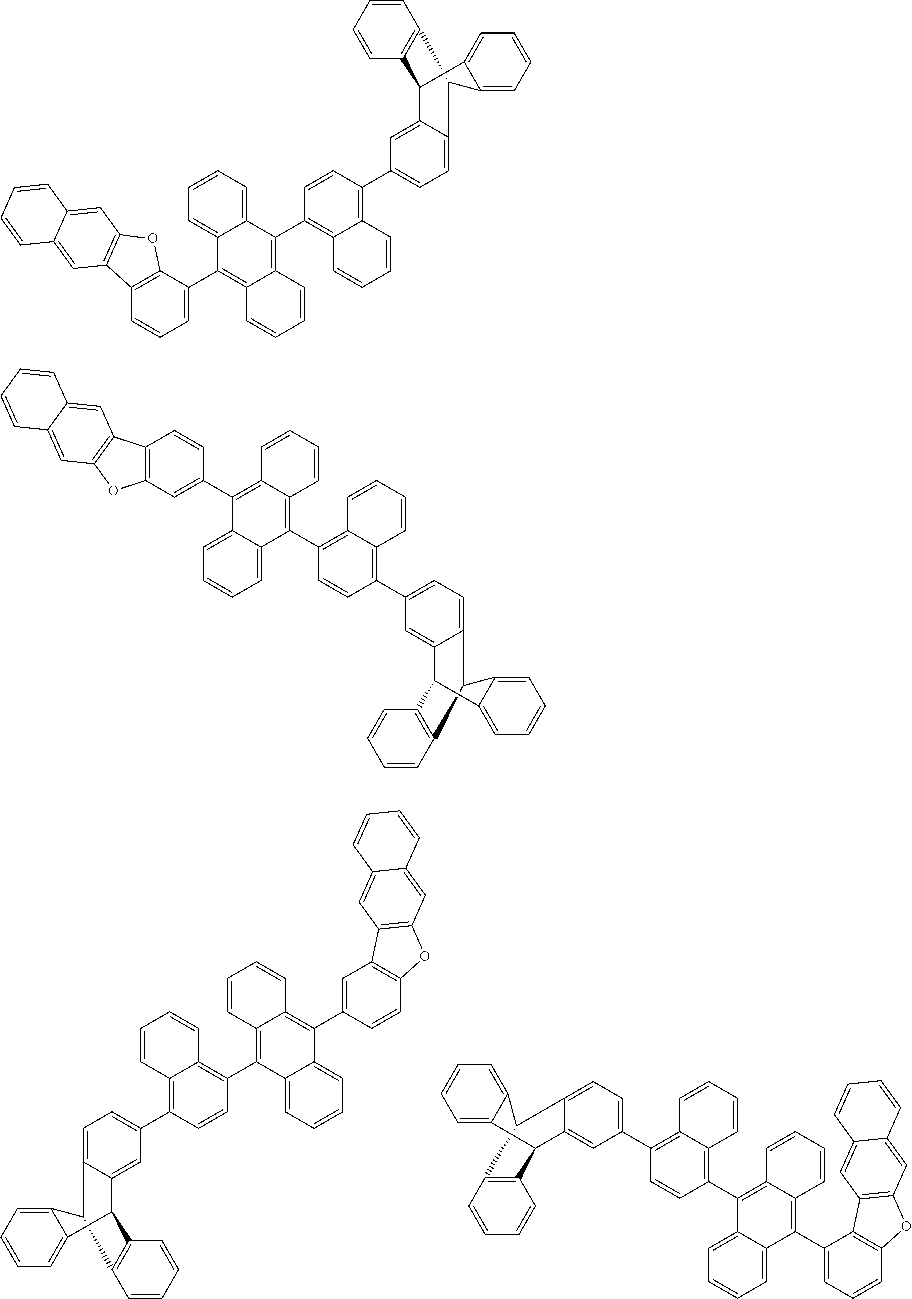

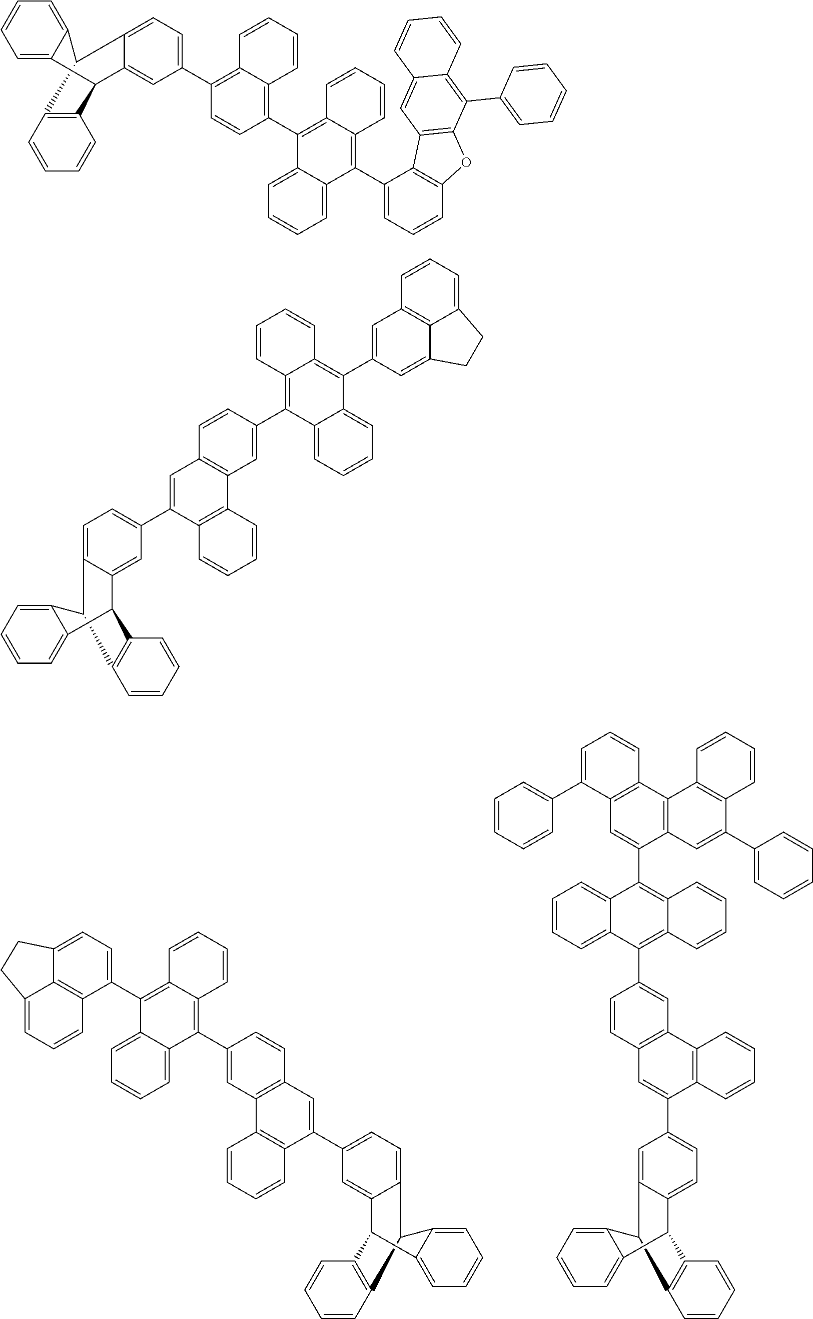

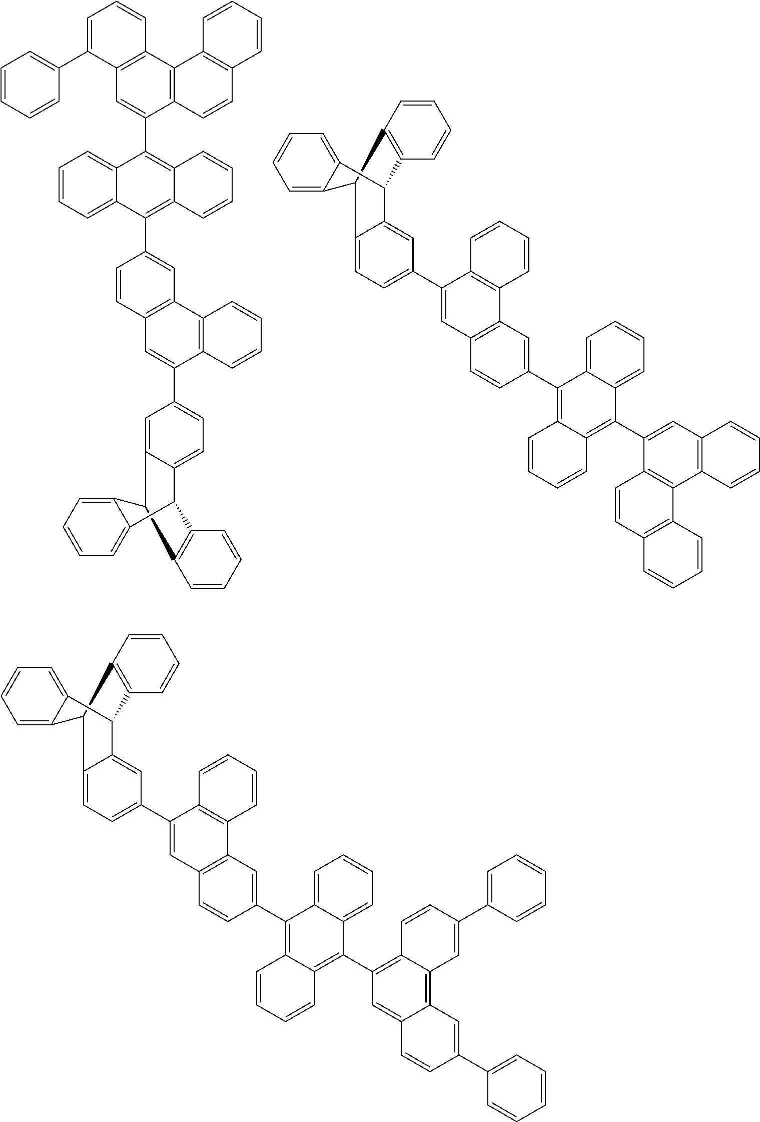

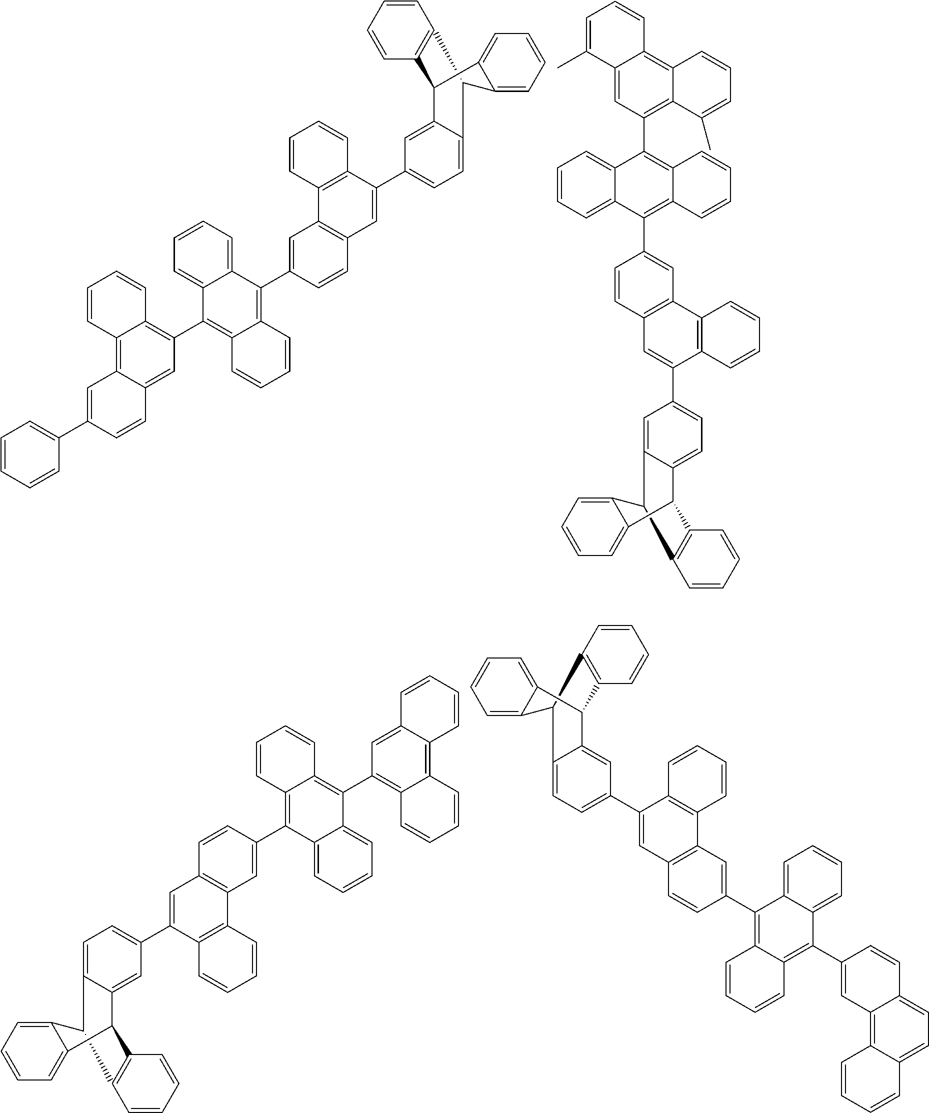

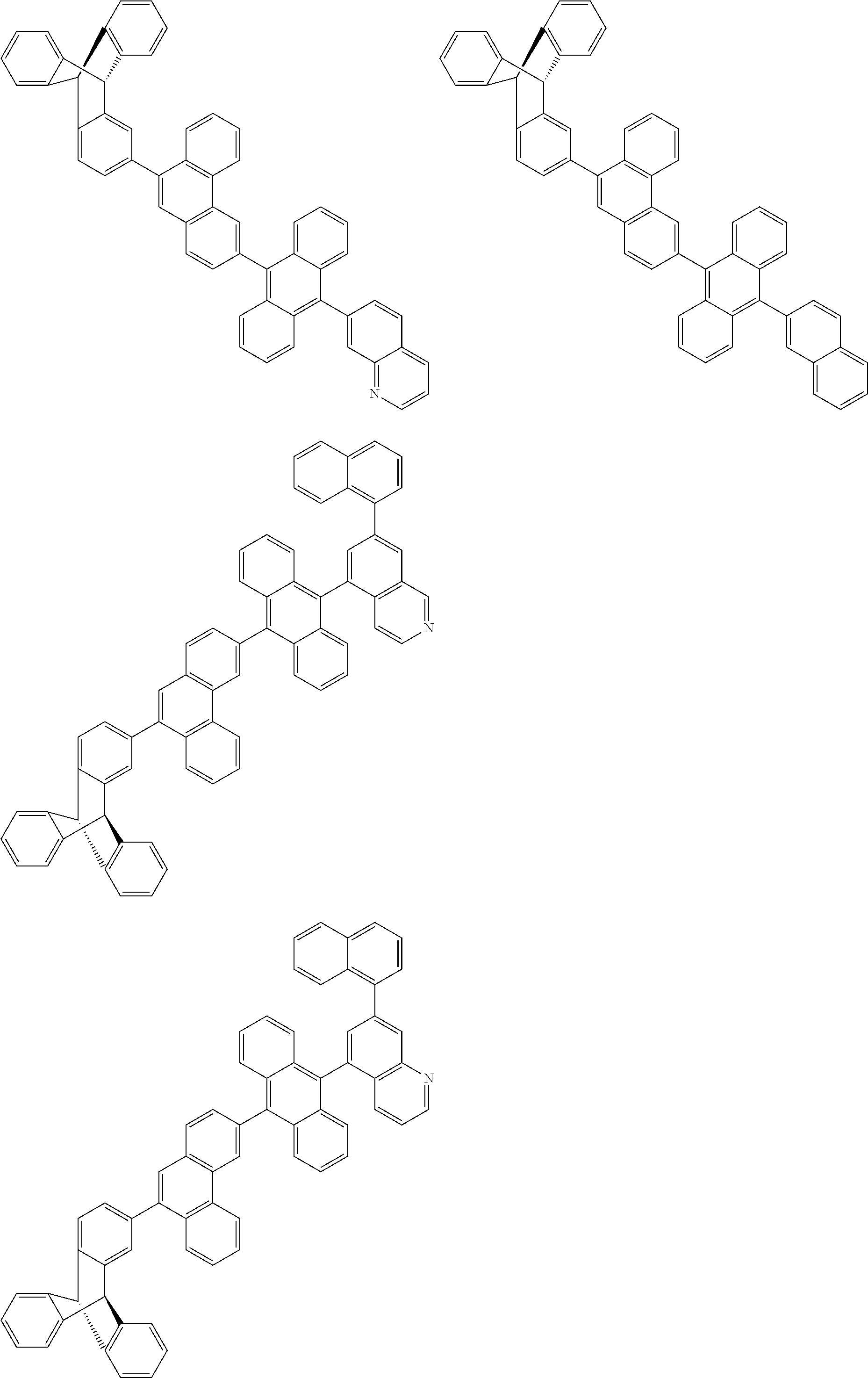

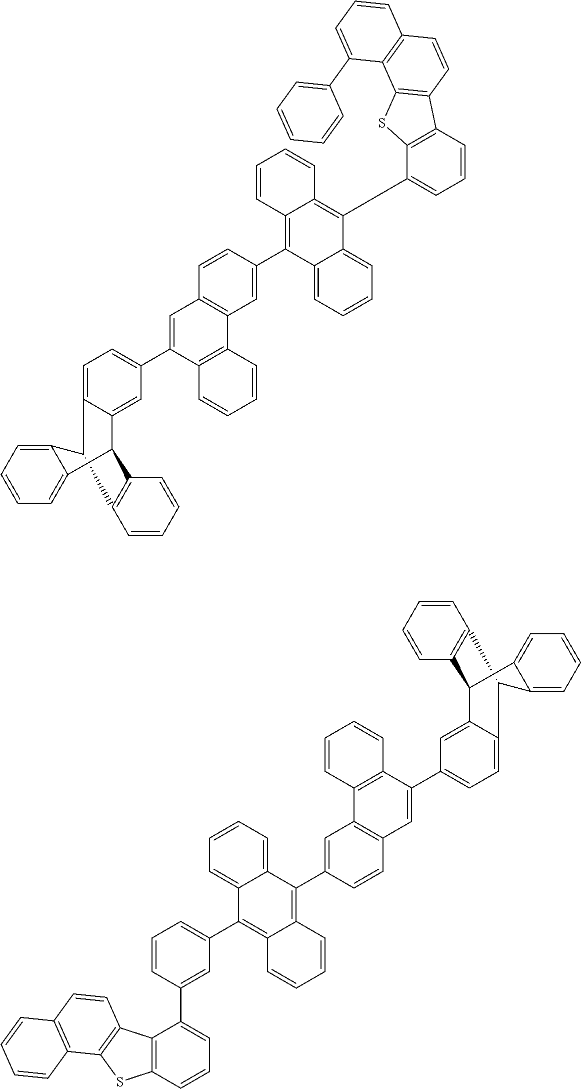 [SMILES: c1ccc(-c2cccc3ccc4c5cccc(-c6c7ccccc7c(-c7ccc8cc(-c9ccc%10c(c9)[C@H]9c%11ccccc%11[C@@H]%10c%10ccccc%109)c9ccccc9c8c7)c7ccccc67)c5sc4c23)cc1 ; c1cc(-c2c3ccccc3c(-c3ccc4cc(-c5ccc6c(c5)[C@H]5c7ccccc7[C@@H]6c6ccccc65)c5ccccc5c4c3)c3ccccc23)cc(-c2cccc3sc4c5ccccc5ccc4c23)c1]

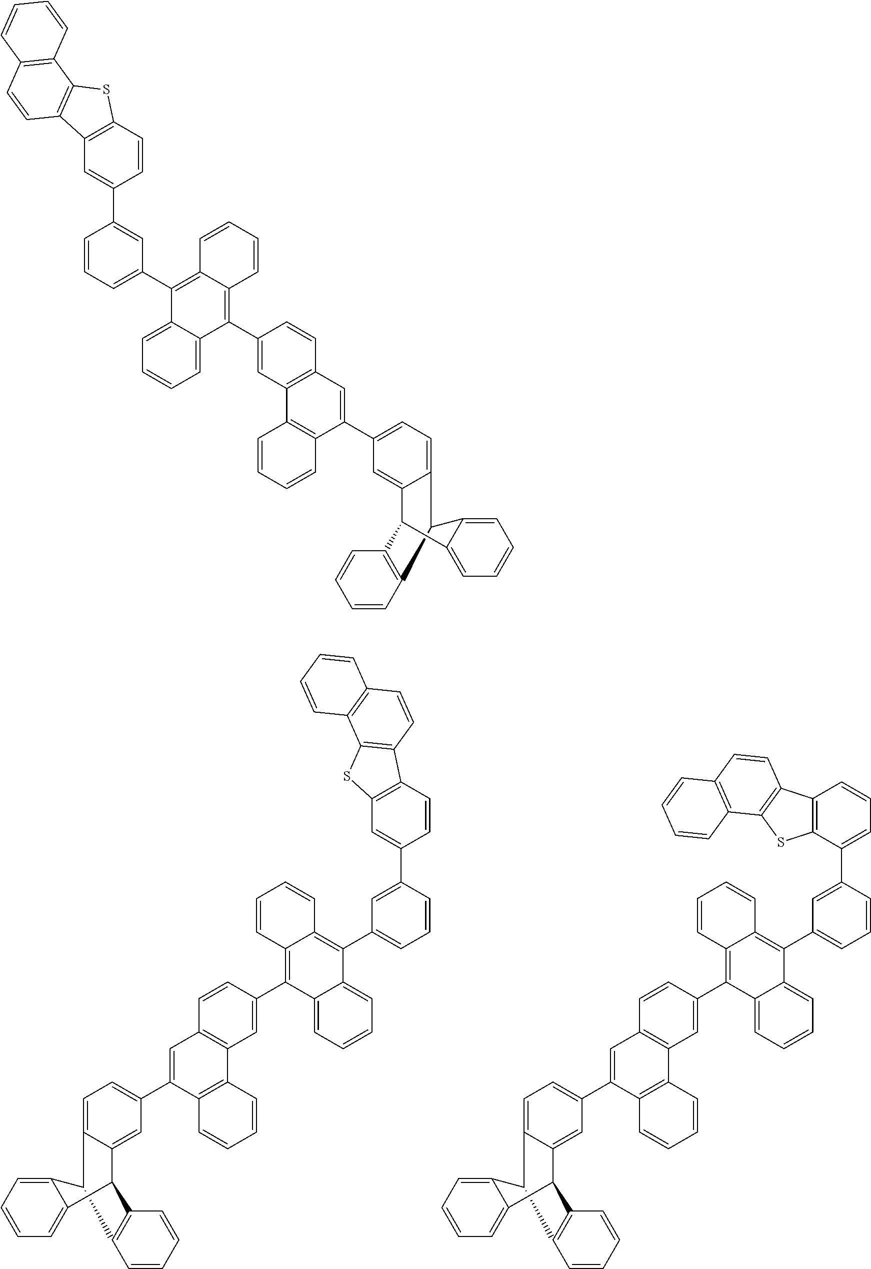 [SMILES: c1cc(-c2ccc3sc4c5ccccc5ccc4c3c2)cc(-c2c3ccccc3c(-c3ccc4cc(-c5ccc6c(c5)[C@H]5c7ccccc7[C@@H]6c6ccccc65)c5ccccc5c4c3)c3ccccc23)c1 ; c1cc(-c2ccc3c(c2)sc2c4ccccc4ccc32)cc(-c2c3ccccc3c(-c3ccc4cc(-c5ccc6c(c5)[C@H]5c7ccccc7[C@@H]6c6ccccc65)c5ccccc5c4c3)c3ccccc23)c1 ; c1cc(-c2c3ccccc3c(-c3ccc4cc(-c5ccc6c(c5)[C@H]5c7ccccc7[C@@H]6c6ccccc65)c5ccccc5c4c3)c3ccccc23)cc(-c2cccc3c2sc2c4ccccc4ccc32)c1]

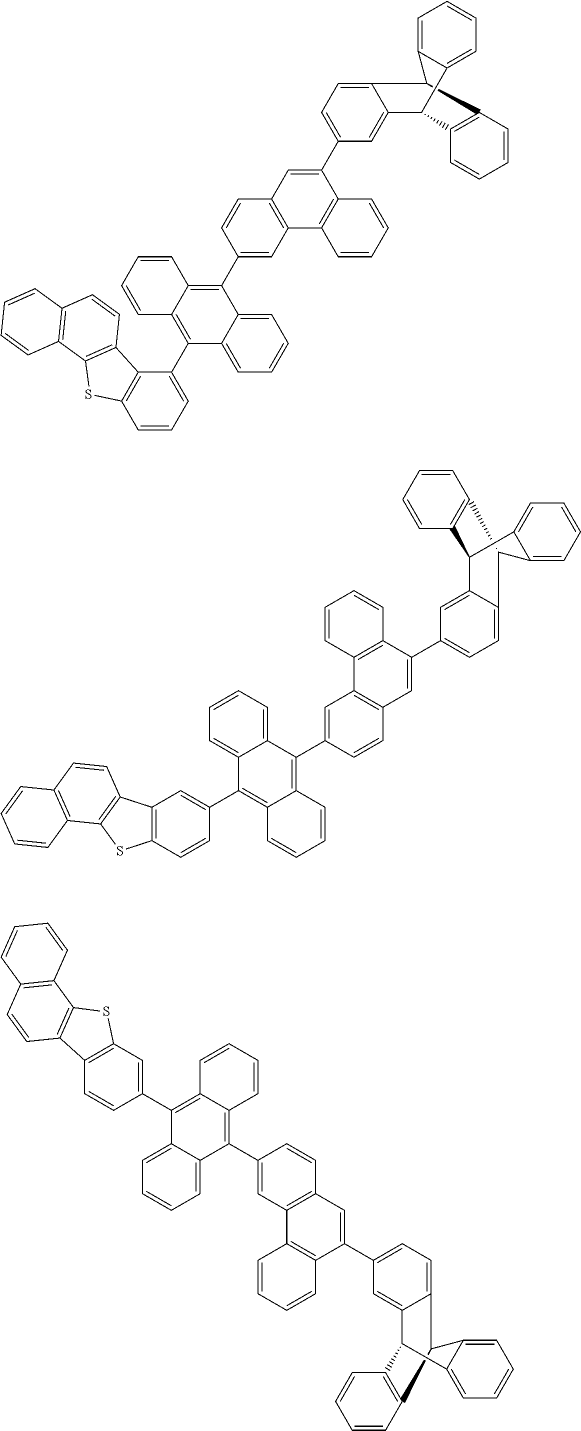 [SMILES: c1ccc2c(c1)ccc1c2sc2cccc(-c3c4ccccc4c(-c4ccc5cc(-c6ccc7c(c6)[C@H]6c8ccccc8[C@@H]7c7ccccc76)c6ccccc6c5c4)c4ccccc34)c21 ; c1ccc2c(c1)ccc1c3cc(-c4c5ccccc5c(-c5ccc6cc(-c7ccc8c(c7)[C@H]7c9ccccc9[C@@H]8c8ccccc87)c7ccccc7c6c5)c5ccccc45)ccc3sc21 ; c1ccc2c(c1)ccc1c3ccc(-c4c5ccccc5c(-c5ccc6cc(-c7ccc8c(c7)[C@H]7c9ccccc9[C@@H]8c8ccccc87)c7ccccc7c6c5)c5ccccc45)cc3sc21]

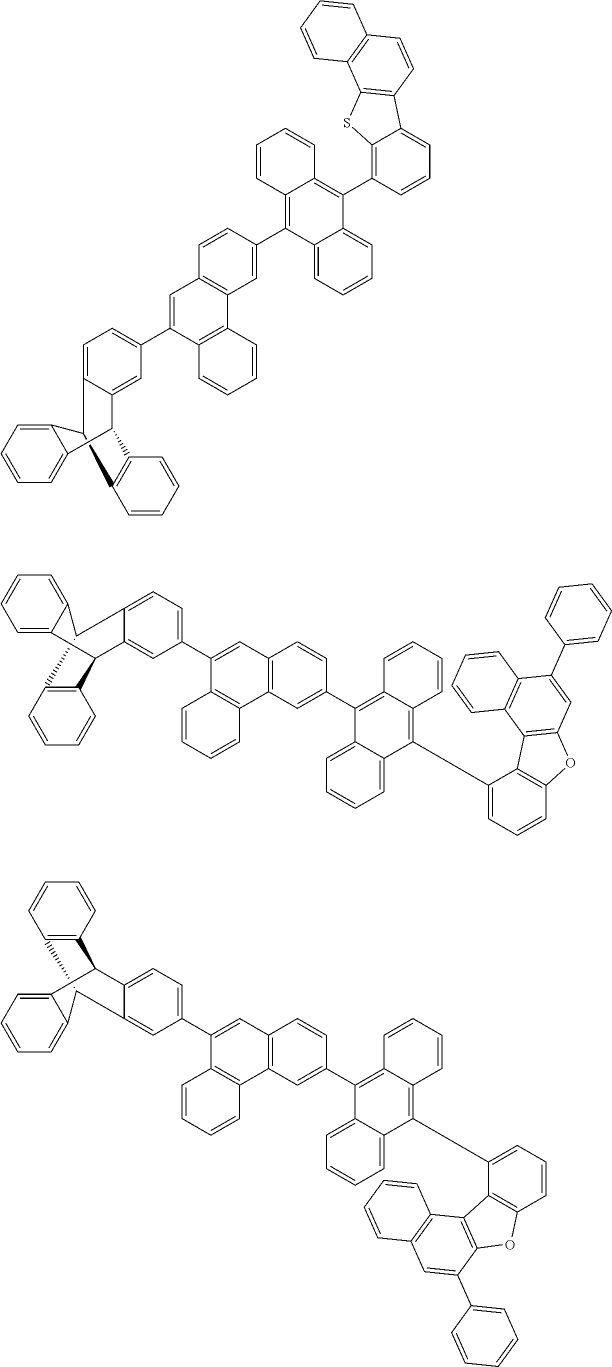 [SMILES: c1ccc2c(c1)ccc1c3cccc(-c4c5ccccc5c(-c5ccc6cc(-c7ccc8c(c7)[C@H]7c9ccccc9[C@@H]8c8ccccc87)c7ccccc7c6c5)c5ccccc45)c3sc21 ; c1ccc(-c2cc3oc4cccc(-c5c6ccccc6c(-c6ccc7cc(-c8ccc9c(c8)[C@H]8c%10ccccc%10[C@@H]9c9ccccc98)c8ccccc8c7c6)c6ccccc56)c4c3c3ccccc23)cc1 ; c1ccc(-c2cc3ccccc3c3c2oc2cccc(-c4c5ccccc5c(-c5ccc6cc(-c7ccc8c(c7)[C@H]7c9ccccc9[C@@H]8c8ccccc87)c7ccccc7c6c5)c5ccccc45)c23)cc1]

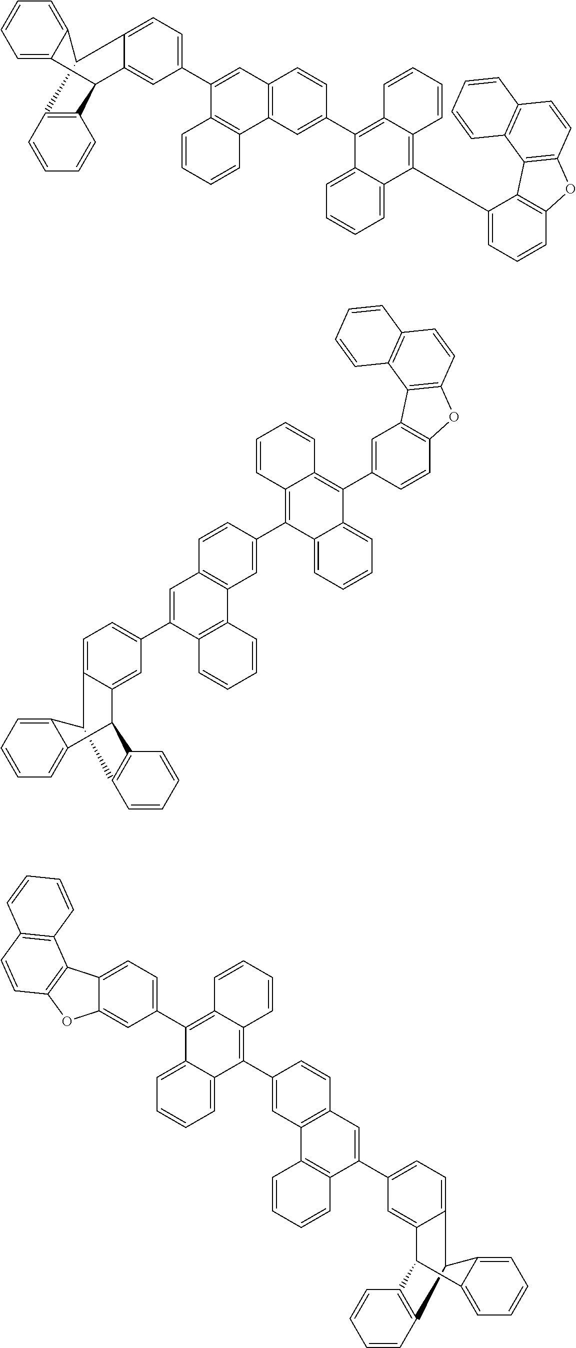 [SMILES: c1ccc2c(c1)ccc1oc3cccc(-c4c5ccccc5c(-c5ccc6cc(-c7ccc8c(c7)[C@H]7c9ccccc9[C@@H]8c8ccccc87)c7ccccc7c6c5)c5ccccc45)c3c12 ; c1ccc2c(c1)ccc1oc3ccc(-c4c5ccccc5c(-c5ccc6cc(-c7ccc8c(c7)[C@H]7c9ccccc9[C@@H]8c8ccccc87)c7ccccc7c6c5)c5ccccc45)cc3c12 ; c1ccc2c(c1)ccc1oc3cc(-c4c5ccccc5c(-c5ccc6cc(-c7ccc8c(c7)[C@H]7c9ccccc9[C@@H]8c8ccccc87)c7ccccc7c6c5)c5ccccc45)ccc3c12]

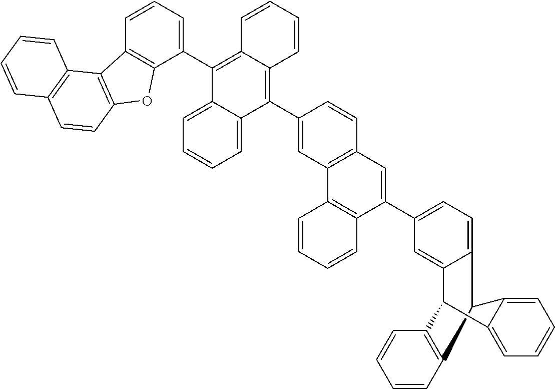 [SMILES: c1ccc2c(c1)ccc1oc3c(-c4c5ccccc5c(-c5ccc6cc(-c7ccc8c(c7)[C@H]7c9ccccc9[C@@H]8c8ccccc87)c7ccccc7c6c5)c5ccccc45)cccc3c12]

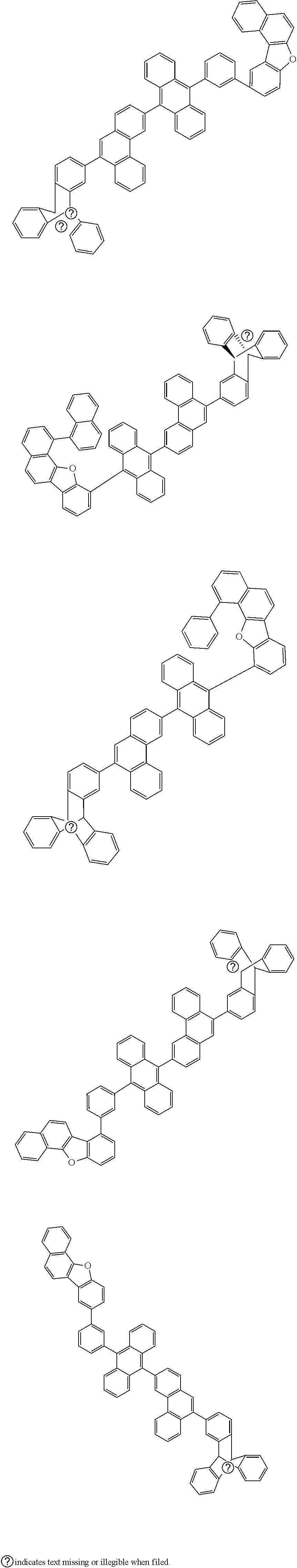

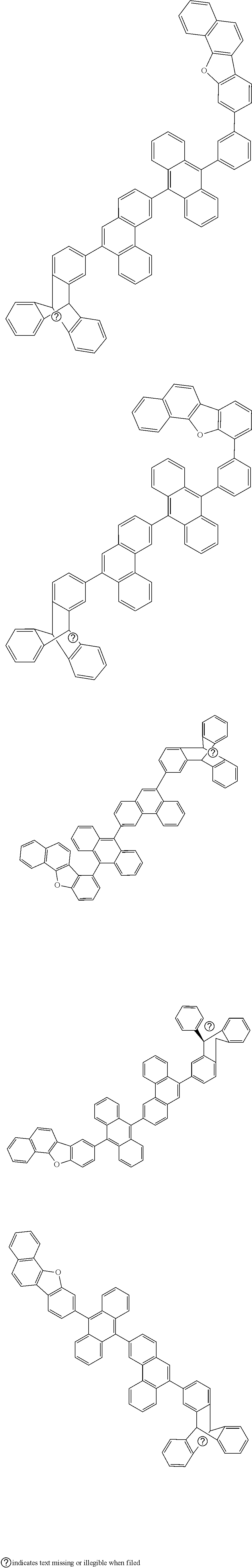

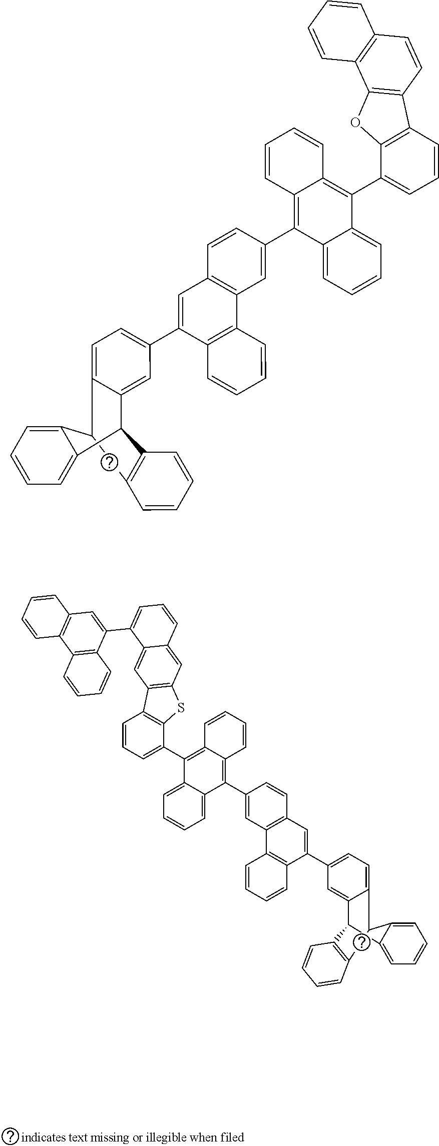

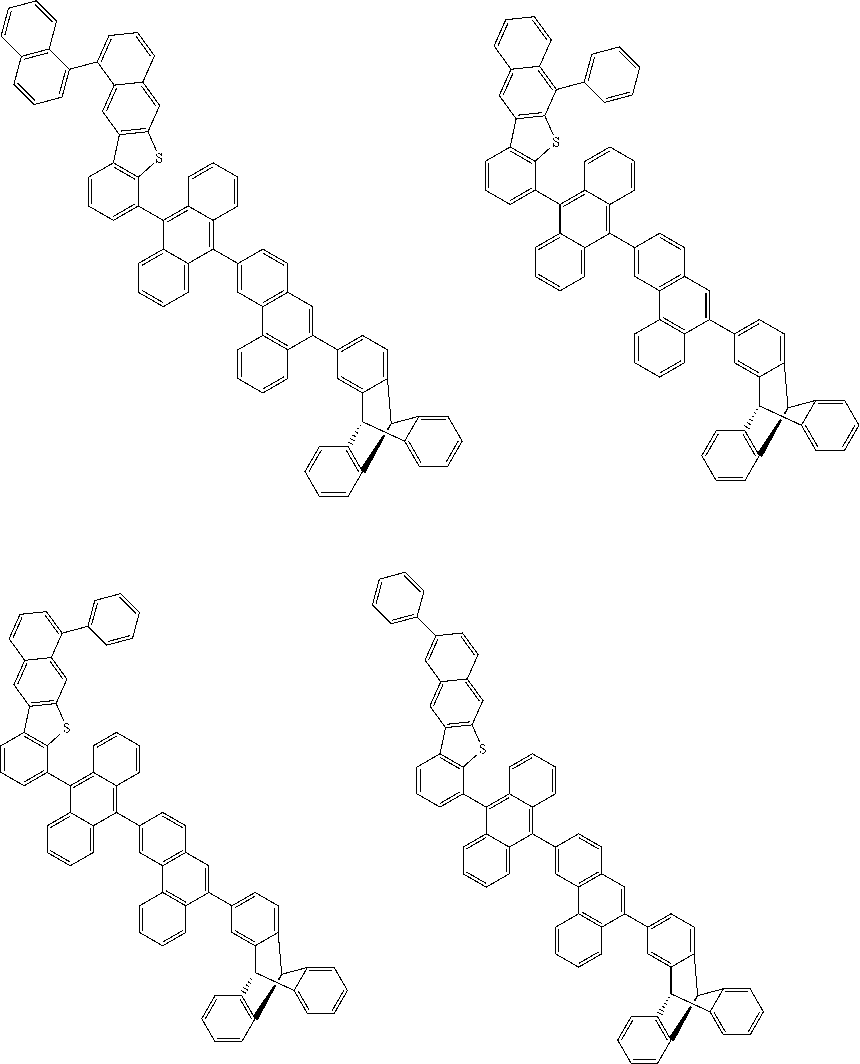

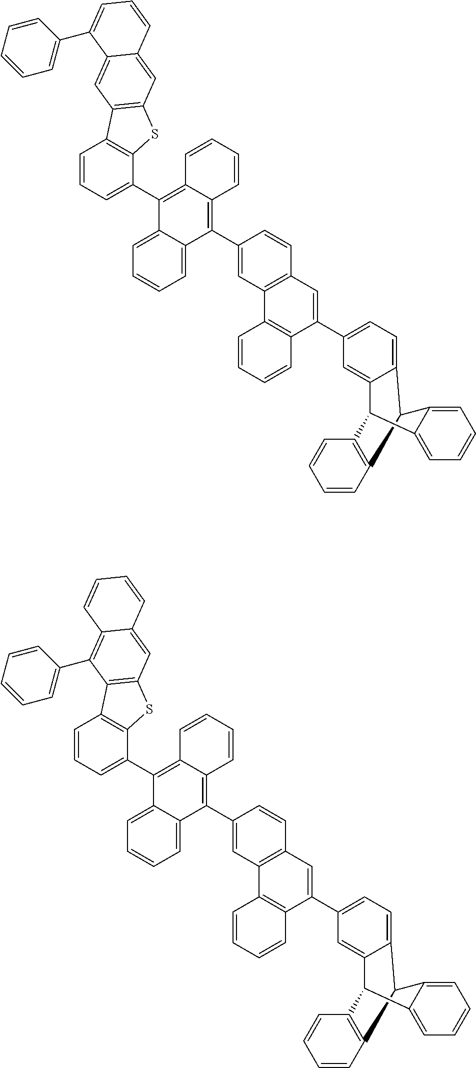 [SMILES: c1ccc(-c2cccc3cc4sc5c(-c6c7ccccc7c(-c7ccc8cc(-c9ccc%10c(c9)[C@H]9c%11ccccc%11[C@@H]%10c%10ccccc%109)c9ccccc9c8c7)c7ccccc67)cccc5c4cc23)cc1 ; c1ccc(-c2c3ccccc3cc3sc4c(-c5c6ccccc6c(-c6ccc7cc(-c8ccc9c(c8)[C@H]8c%10ccccc%10[C@@H]9c9ccccc98)c8ccccc8c7c6)c6ccccc56)cccc4c23)cc1]

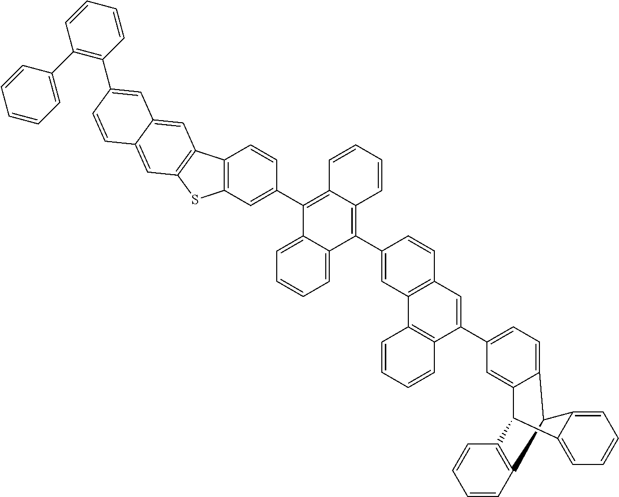 [SMILES: c1ccc(-c2ccccc2-c2ccc3cc4sc5cc(-c6c7ccccc7c(-c7ccc8cc(-c9ccc%10c(c9)[C@H]9c%11ccccc%11[C@@H]%10c%10ccccc%109)c9ccccc9c8c7)c7ccccc67)ccc5c4cc3c2)cc1]

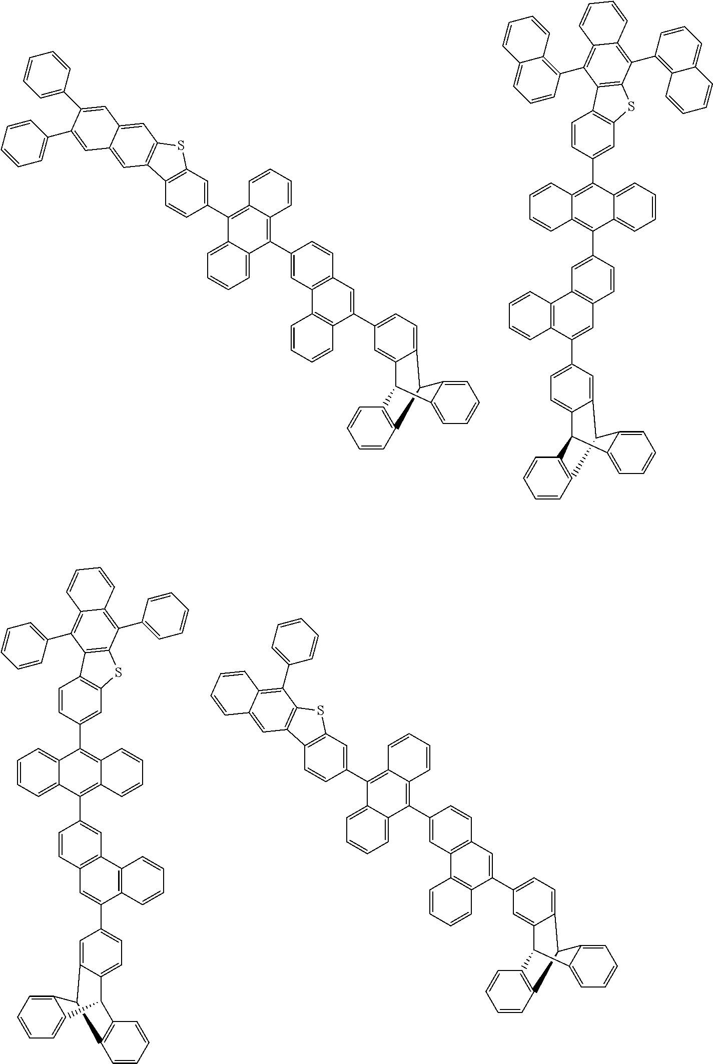

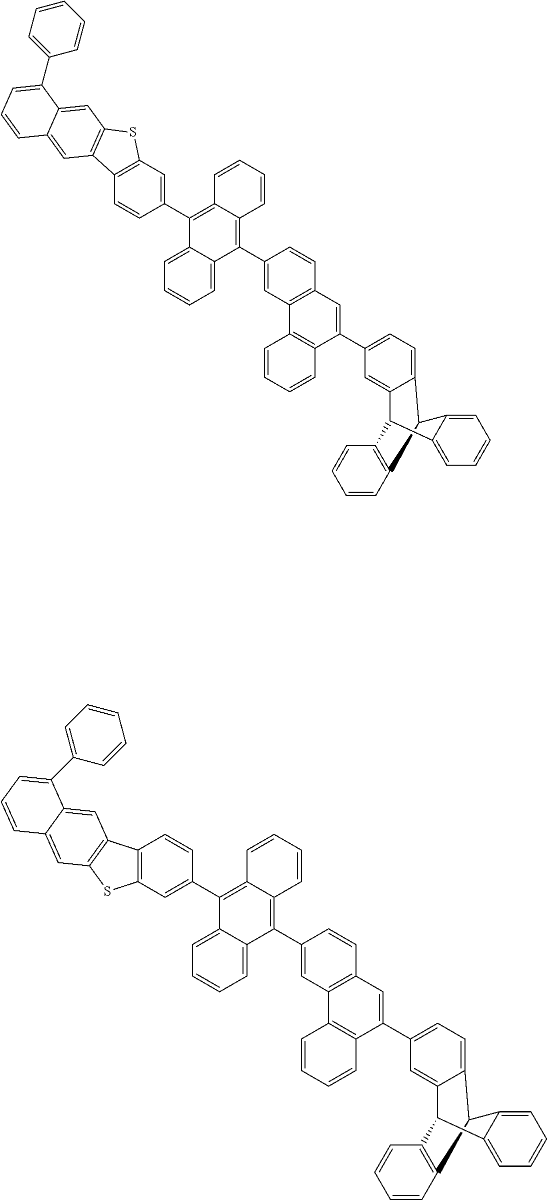

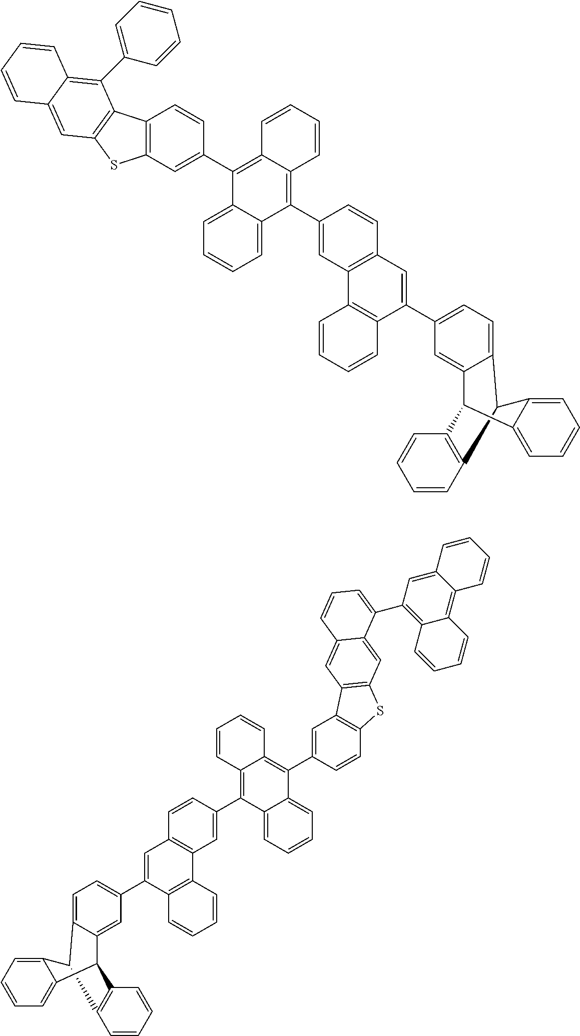 [SMILES: c1ccc(-c2c3ccccc3cc3sc4cc(-c5c6ccccc6c(-c6ccc7cc(-c8ccc9c(c8)[C@H]8c%10ccccc%10[C@@H]9c9ccccc98)c8ccccc8c7c6)c6ccccc56)ccc4c23)cc1 ; c1cc(-c2cc3ccccc3c3ccccc23)c2cc3sc4ccc(-c5c6ccccc6c(-c6ccc7cc(-c8ccc9c(c8)[C@H]8c%10ccccc%10[C@@H]9c9ccccc98)c8ccccc8c7c6)c6ccccc56)cc4c3cc2c1]

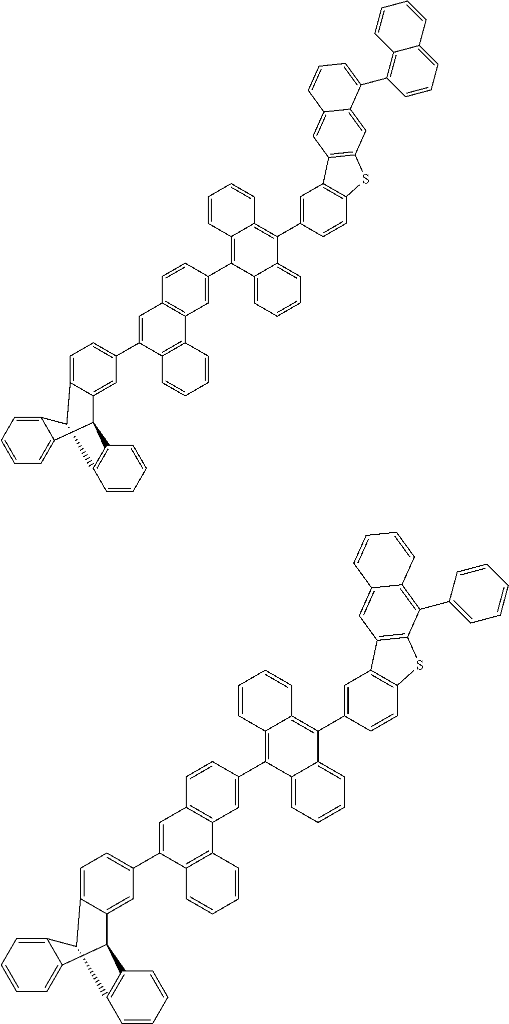 [SMILES: c1ccc2c(-c3cccc4cc5c(cc34)sc3ccc(-c4c6ccccc6c(-c6ccc7cc(-c8ccc9c(c8)[C@H]8c%10ccccc%10[C@@H]9c9ccccc98)c8ccccc8c7c6)c6ccccc46)cc35)cccc2c1 ; c1ccc(-c2c3ccccc3cc3c2sc2ccc(-c4c5ccccc5c(-c5ccc6cc(-c7ccc8c(c7)[C@H]7c9ccccc9[C@@H]8c8ccccc87)c7ccccc7c6c5)c5ccccc45)cc23)cc1]

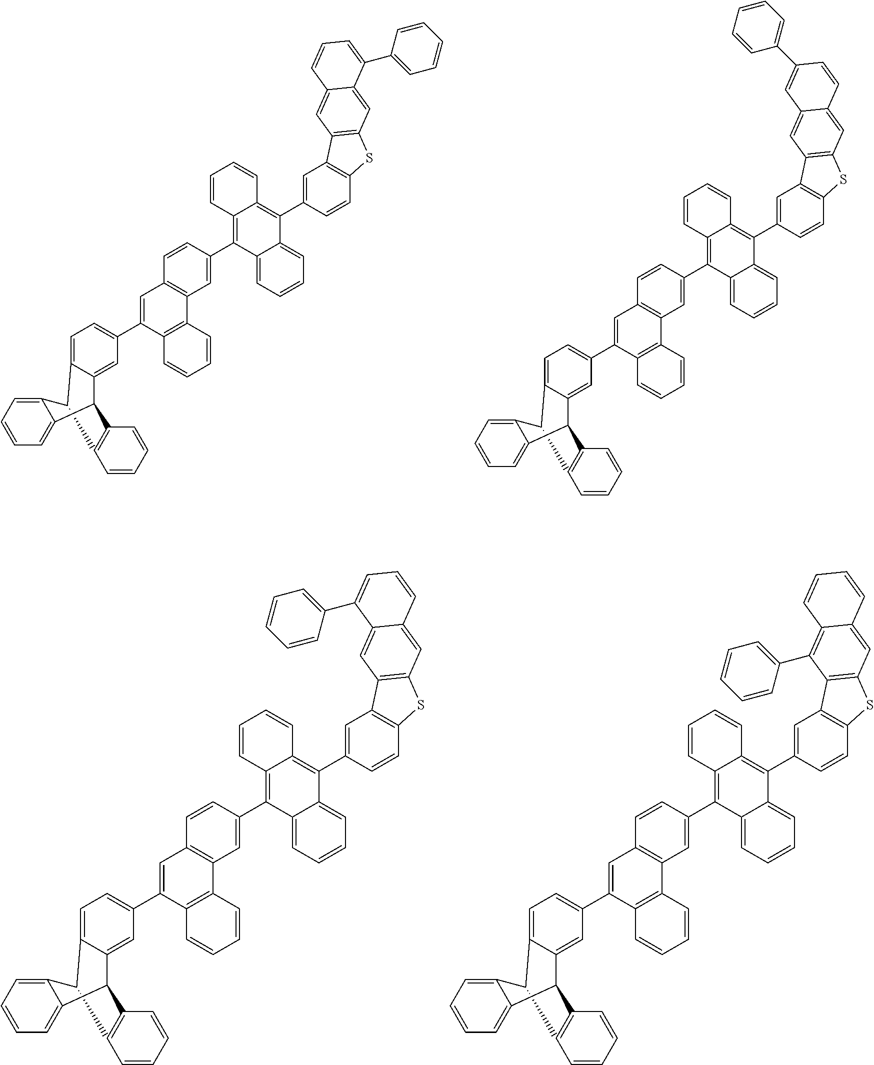

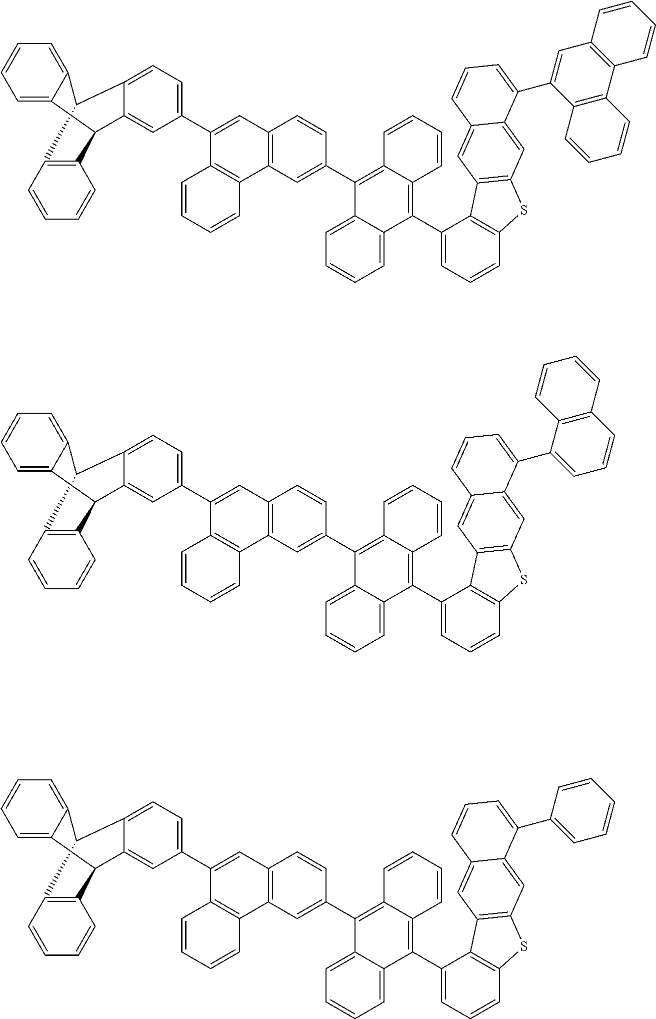 [SMILES: c1cc(-c2cc3ccccc3c3ccccc23)c2cc3sc4cccc(-c5c6ccccc6c(-c6ccc7cc(-c8ccc9c(c8)[C@H]8c%10ccccc%10[C@@H]9c9ccccc98)c8ccccc8c7c6)c6ccccc56)c4c3cc2c1 ; c1ccc2c(-c3cccc4cc5c(cc34)sc3cccc(-c4c6ccccc6c(-c6ccc7cc(-c8ccc9c(c8)[C@H]8c%10ccccc%10[C@@H]9c9ccccc98)c8ccccc8c7c6)c6ccccc46)c35)cccc2c1 ; c1ccc(-c2cccc3cc4c(cc23)sc2cccc(-c3c5ccccc5c(-c5ccc6cc(-c7ccc8c(c7)[C@H]7c9ccccc9[C@@H]8c8ccccc87)c7ccccc7c6c5)c5ccccc35)c24)cc1]

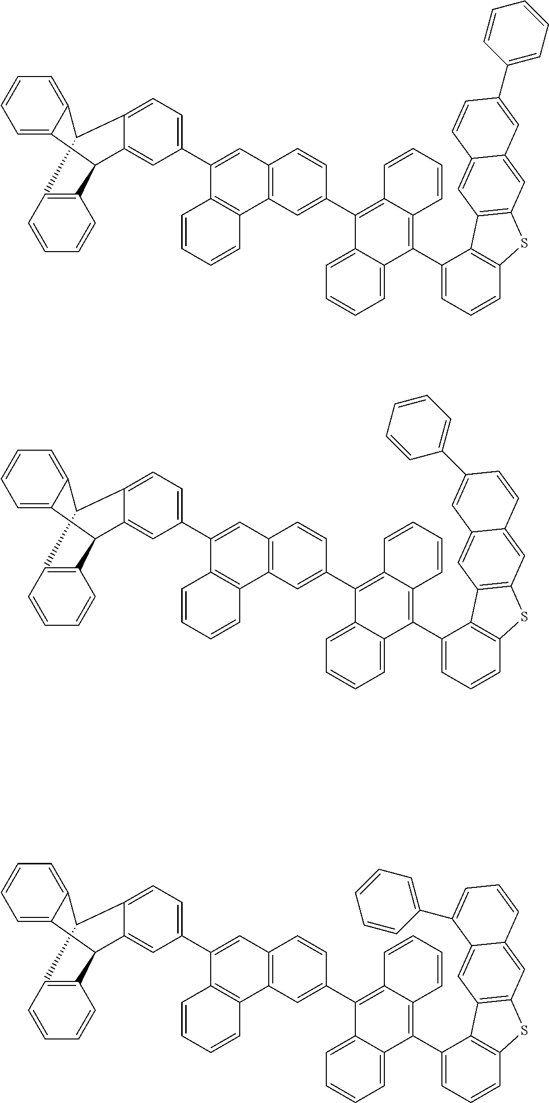 [SMILES: c1ccc(-c2ccc3cc4c(cc3c2)sc2cccc(-c3c5ccccc5c(-c5ccc6cc(-c7ccc8c(c7)[C@H]7c9ccccc9[C@@H]8c8ccccc87)c7ccccc7c6c5)c5ccccc35)c24)cc1 ; c1ccc(-c2ccc3cc4sc5cccc(-c6c7ccccc7c(-c7ccc8cc(-c9ccc%10c(c9)[C@H]9c%11ccccc%11[C@@H]%10c%10ccccc%109)c9ccccc9c8c7)c7ccccc67)c5c4cc3c2)cc1 ; c1ccc(-c2cccc3cc4sc5cccc(-c6c7ccccc7c(-c7ccc8cc(-c9ccc%10c(c9)[C@H]9c%11ccccc%11[C@@H]%10c%10ccccc%109)c9ccccc9c8c7)c7ccccc67)c5c4cc23)cc1]

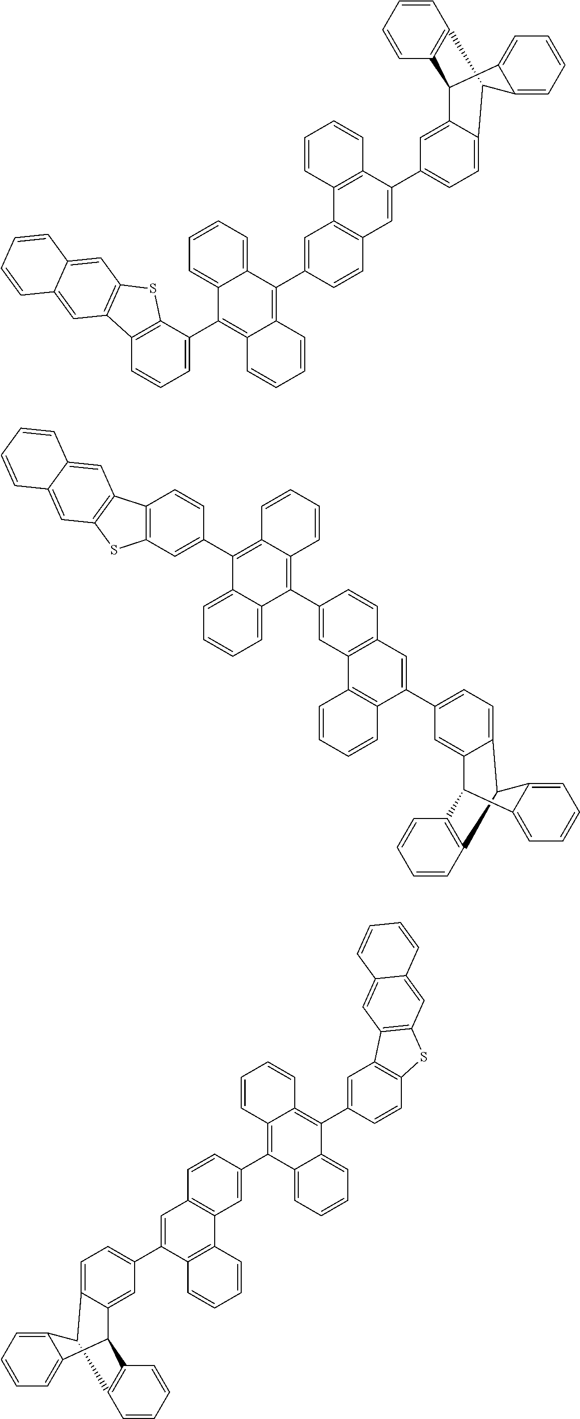 [SMILES: c1ccc2cc3c(cc2c1)sc1c(-c2c4ccccc4c(-c4ccc5cc(-c6ccc7c(c6)[C@H]6c8ccccc8[C@@H]7c7ccccc76)c6ccccc6c5c4)c4ccccc24)cccc13 ; c1ccc2cc3c(cc2c1)sc1cc(-c2c4ccccc4c(-c4ccc5cc(-c6ccc7c(c6)[C@H]6c8ccccc8[C@@H]7c7ccccc76)c6ccccc6c5c4)c4ccccc24)ccc13 ; c1ccc2cc3c(cc2c1)sc1ccc(-c2c4ccccc4c(-c4ccc5cc(-c6ccc7c(c6)[C@H]6c8ccccc8[C@@H]7c7ccccc76)c6ccccc6c5c4)c4ccccc24)cc13]

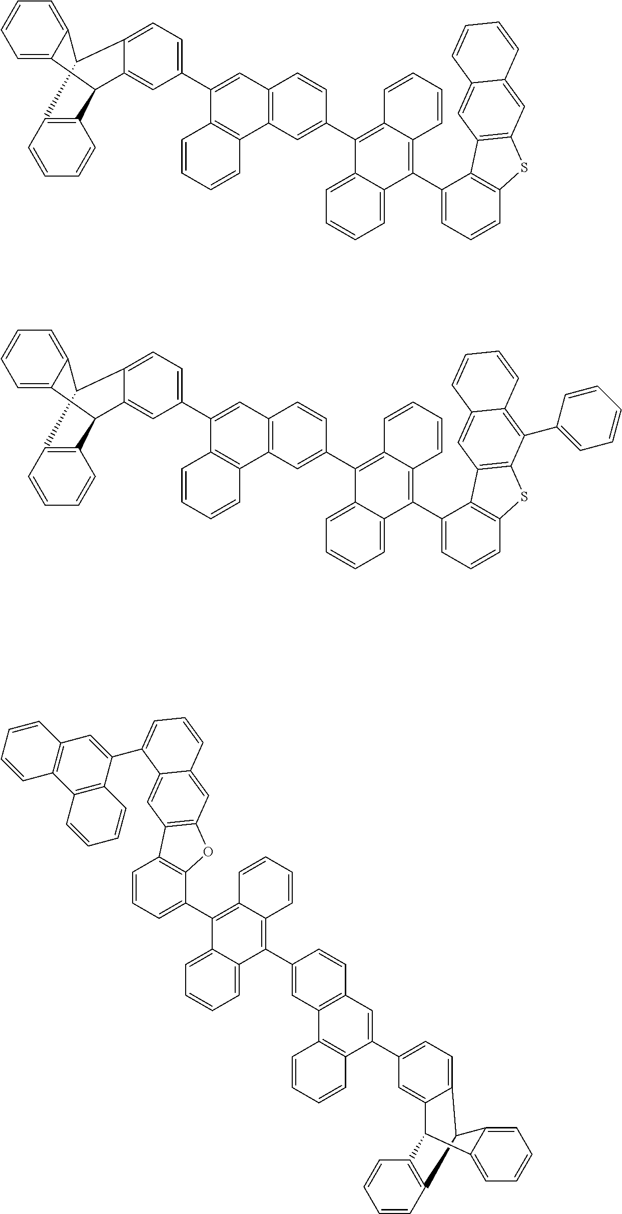 [SMILES: c1ccc2cc3c(cc2c1)sc1cccc(-c2c4ccccc4c(-c4ccc5cc(-c6ccc7c(c6)[C@H]6c8ccccc8[C@@H]7c7ccccc76)c6ccccc6c5c4)c4ccccc24)c13 ; c1ccc(-c2c3ccccc3cc3c2sc2cccc(-c4c5ccccc5c(-c5ccc6cc(-c7ccc8c(c7)[C@H]7c9ccccc9[C@@H]8c8ccccc87)c7ccccc7c6c5)c5ccccc45)c23)cc1 ; c1cc(-c2cc3ccccc3c3ccccc23)c2cc3c(cc2c1)oc1c(-c2c4ccccc4c(-c4ccc5cc(-c6ccc7c(c6)[C@H]6c8ccccc8[C@@H]7c7ccccc76)c6ccccc6c5c4)c4ccccc24)cccc13]

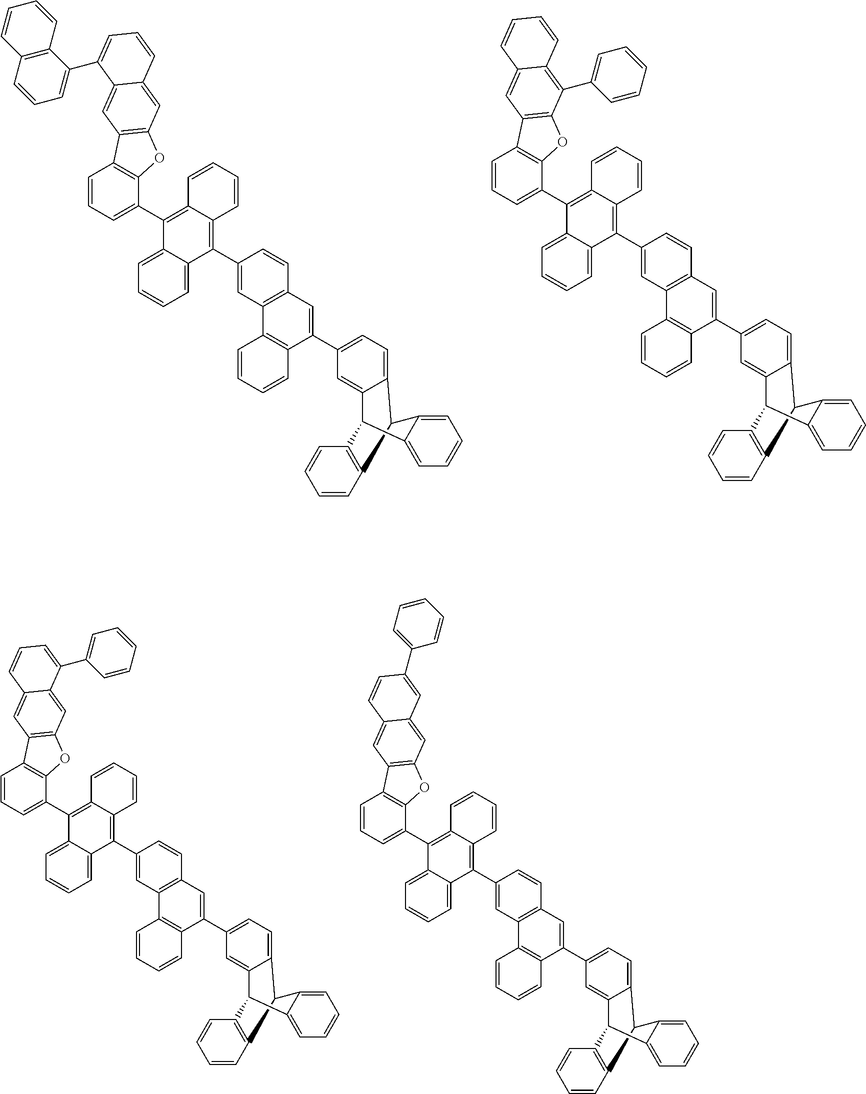

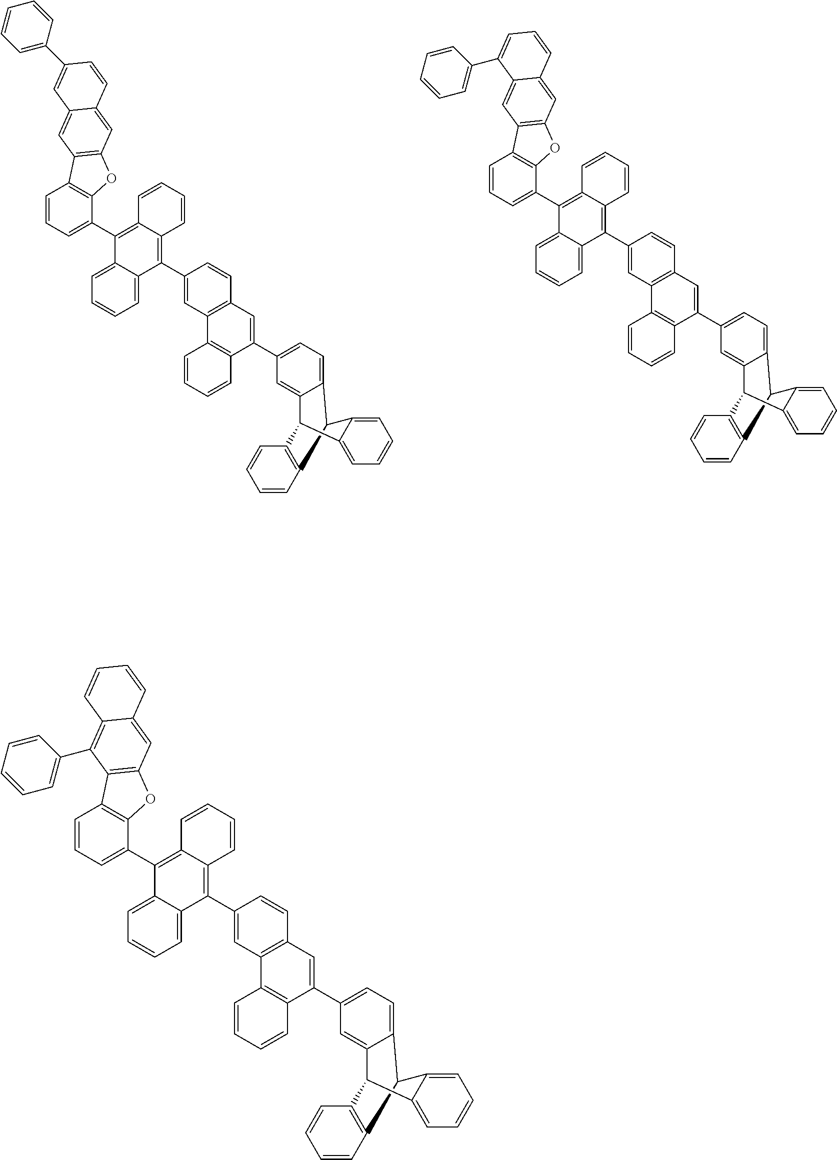 [SMILES: c1ccc(-c2ccc3cc4oc5c(-c6c7ccccc7c(-c7ccc8cc(-c9ccc%10c(c9)[C@H]9c%11ccccc%11[C@@H]%10c%10ccccc%109)c9ccccc9c8c7)c7ccccc67)cccc5c4cc3c2)cc1 ; c1ccc(-c2cccc3cc4oc5c(-c6c7ccccc7c(-c7ccc8cc(-c9ccc%10c(c9)[C@H]9c%11ccccc%11[C@@H]%10c%10ccccc%109)c9ccccc9c8c7)c7ccccc67)cccc5c4cc23)cc1 ; c1ccc(-c2c3ccccc3cc3oc4c(-c5c6ccccc6c(-c6ccc7cc(-c8ccc9c(c8)[C@H]8c%10ccccc%10[C@@H]9c9ccccc98)c8ccccc8c7c6)c6ccccc56)cccc4c23)cc1]

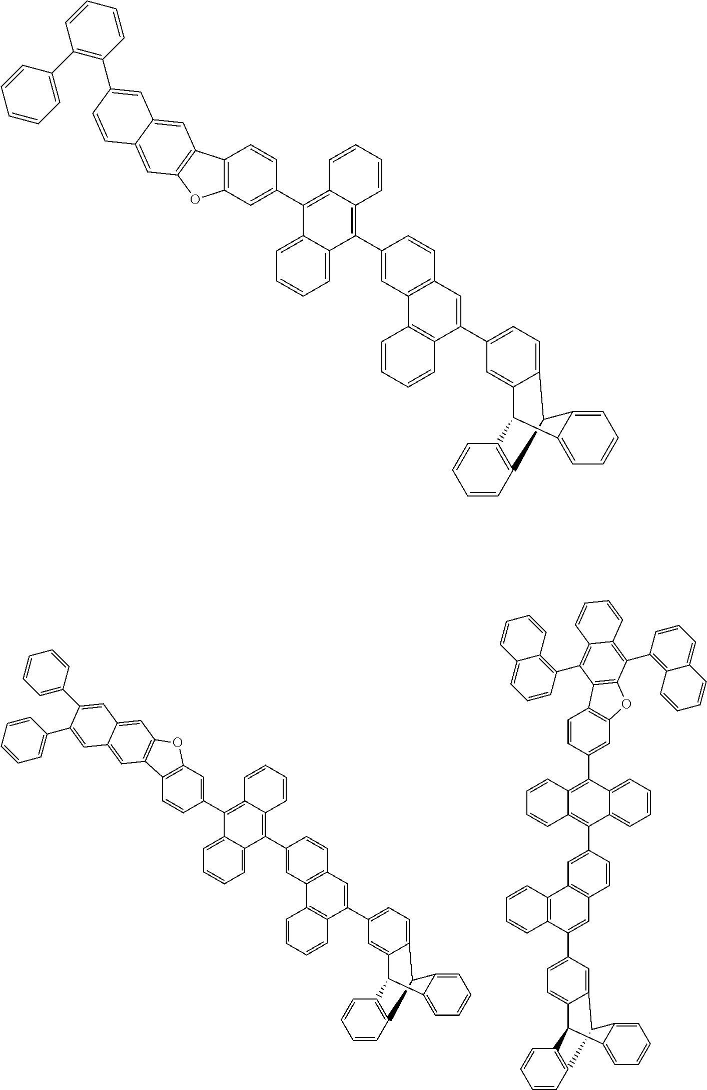 [SMILES: c1ccc(-c2ccccc2-c2ccc3cc4oc5cc(-c6c7ccccc7c(-c7ccc8cc(-c9ccc%10c(c9)[C@H]9c%11ccccc%11[C@@H]%10c%10ccccc%109)c9ccccc9c8c7)c7ccccc67)ccc5c4cc3c2)cc1 ; c1ccc(-c2cc3cc4oc5cc(-c6c7ccccc7c(-c7ccc8cc(-c9ccc%10c(c9)[C@H]9c%11ccccc%11[C@@H]%10c%10ccccc%109)c9ccccc9c8c7)c7ccccc67)ccc5c4cc3cc2-c2ccccc2)cc1 ; c1ccc2c(-c3c4ccccc4c(-c4cccc5ccccc45)c4c3oc3cc(-c5c6ccccc6c(-c6ccc7cc(-c8ccc9c(c8)[C@H]8c%10ccccc%10[C@@H]9c9ccccc98)c8ccccc8c7c6)c6ccccc56)ccc34)cccc2c1]

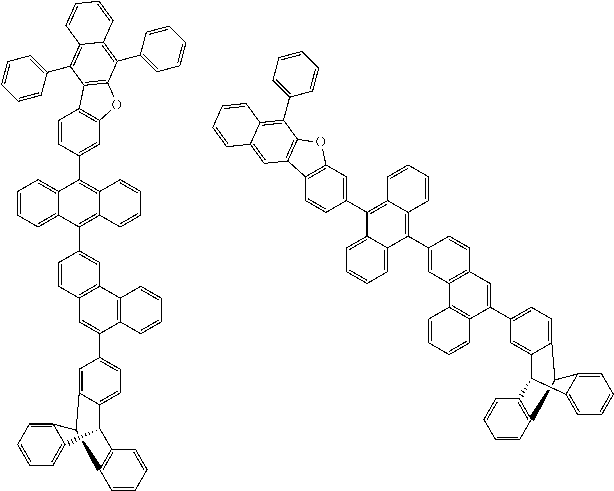 [SMILES: c1ccc(-c2c3ccccc3c(-c3ccccc3)c3c2oc2cc(-c4c5ccccc5c(-c5ccc6cc(-c7ccc8c(c7)[C@H]7c9ccccc9[C@@H]8c8ccccc87)c7ccccc7c6c5)c5ccccc45)ccc23)cc1 ; c1ccc(-c2c3ccccc3cc3c2oc2cc(-c4c5ccccc5c(-c5ccc6cc(-c7ccc8c(c7)[C@H]7c9ccccc9[C@@H]8c8ccccc87)c7ccccc7c6c5)c5ccccc45)ccc23)cc1]

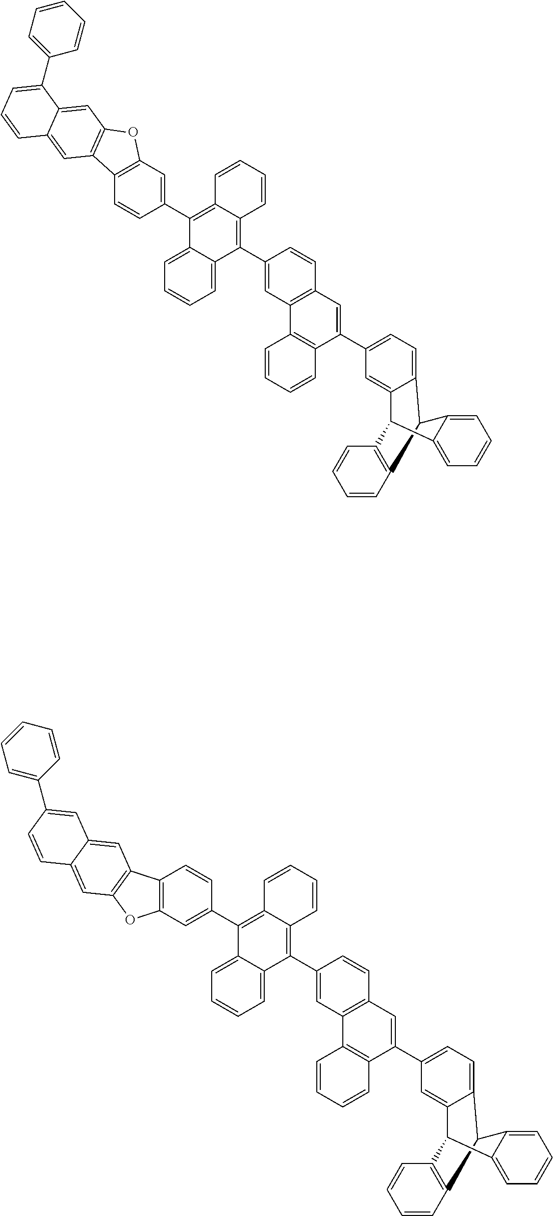 [SMILES: c1ccc(-c2cccc3cc4c(cc23)oc2cc(-c3c5ccccc5c(-c5ccc6cc(-c7ccc8c(c7)[C@H]7c9ccccc9[C@@H]8c8ccccc87)c7ccccc7c6c5)c5ccccc35)ccc24)cc1 ; c1ccc(-c2ccc3cc4oc5cc(-c6c7ccccc7c(-c7ccc8cc(-c9ccc%10c(c9)[C@H]9c%11ccccc%11[C@@H]%10c%10ccccc%109)c9ccccc9c8c7)c7ccccc67)ccc5c4cc3c2)cc1]

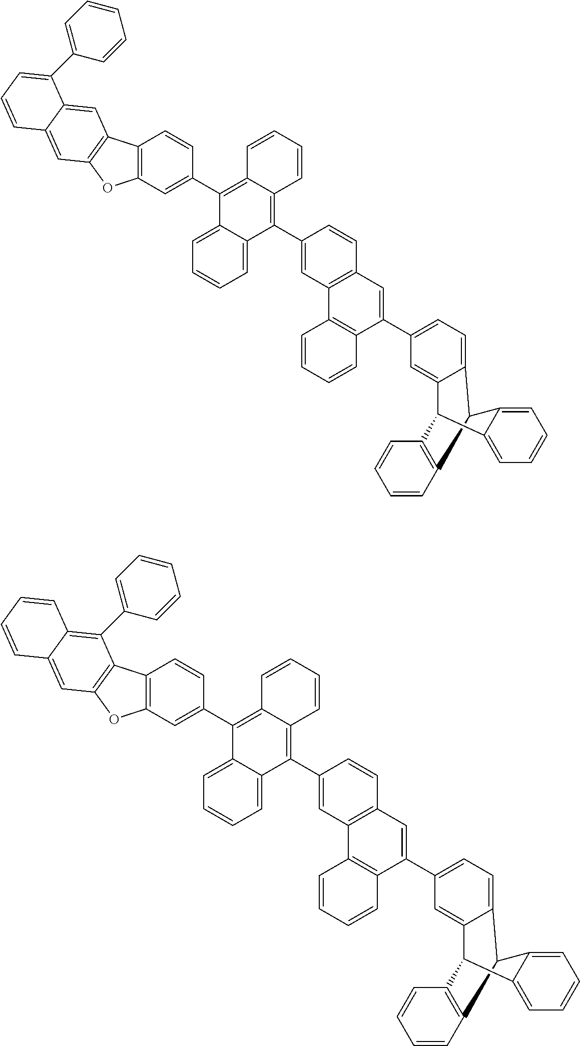 [SMILES: c1ccc(-c2cccc3cc4oc5cc(-c6c7ccccc7c(-c7ccc8cc(-c9ccc%10c(c9)[C@H]9c%11ccccc%11[C@@H]%10c%10ccccc%109)c9ccccc9c8c7)c7ccccc67)ccc5c4cc23)cc1 ; c1ccc(-c2c3ccccc3cc3oc4cc(-c5c6ccccc6c(-c6ccc7cc(-c8ccc9c(c8)[C@H]8c%10ccccc%10[C@@H]9c9ccccc98)c8ccccc8c7c6)c6ccccc56)ccc4c23)cc1]

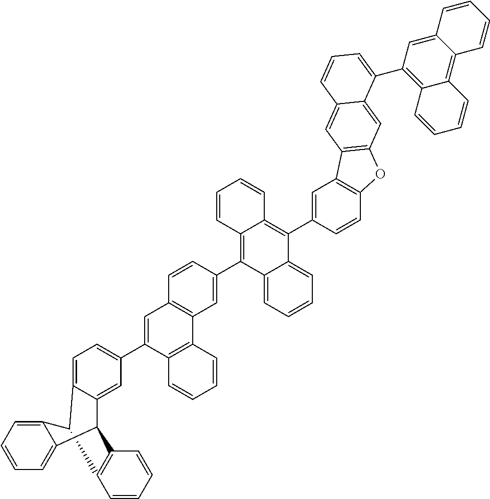 [SMILES: c1cc(-c2cc3ccccc3c3ccccc23)c2cc3oc4ccc(-c5c6ccccc6c(-c6ccc7cc(-c8ccc9c(c8)[C@H]8c%10ccccc%10[C@@H]9c9ccccc98)c8ccccc8c7c6)c6ccccc56)cc4c3cc2c1]

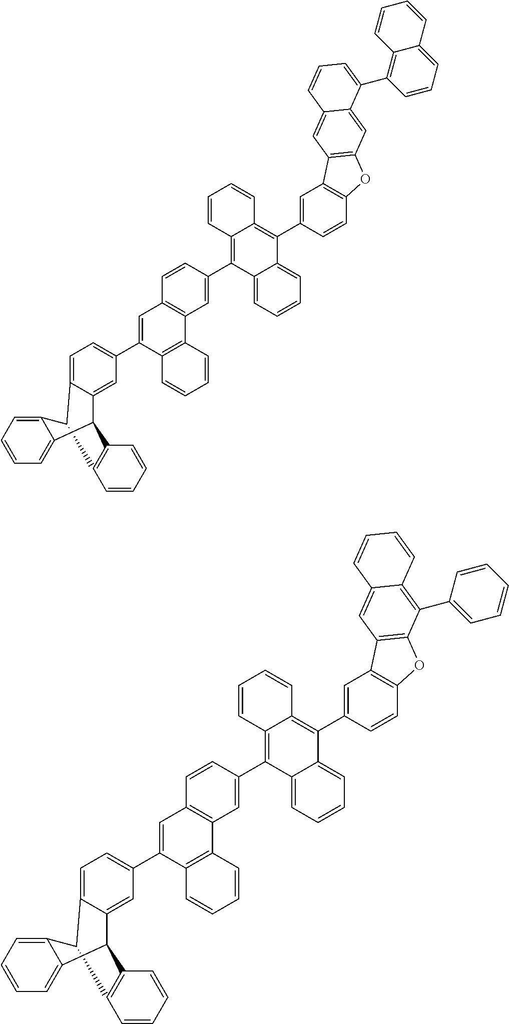 [SMILES: c1ccc2c(-c3cccc4cc5c(cc34)oc3ccc(-c4c6ccccc6c(-c6ccc7cc(-c8ccc9c(c8)[C@H]8c%10ccccc%10[C@@H]9c9ccccc98)c8ccccc8c7c6)c6ccccc46)cc35)cccc2c1 ; c1ccc(-c2c3ccccc3cc3c2oc2ccc(-c4c5ccccc5c(-c5ccc6cc(-c7ccc8c(c7)[C@H]7c9ccccc9[C@@H]8c8ccccc87)c7ccccc7c6c5)c5ccccc45)cc23)cc1]

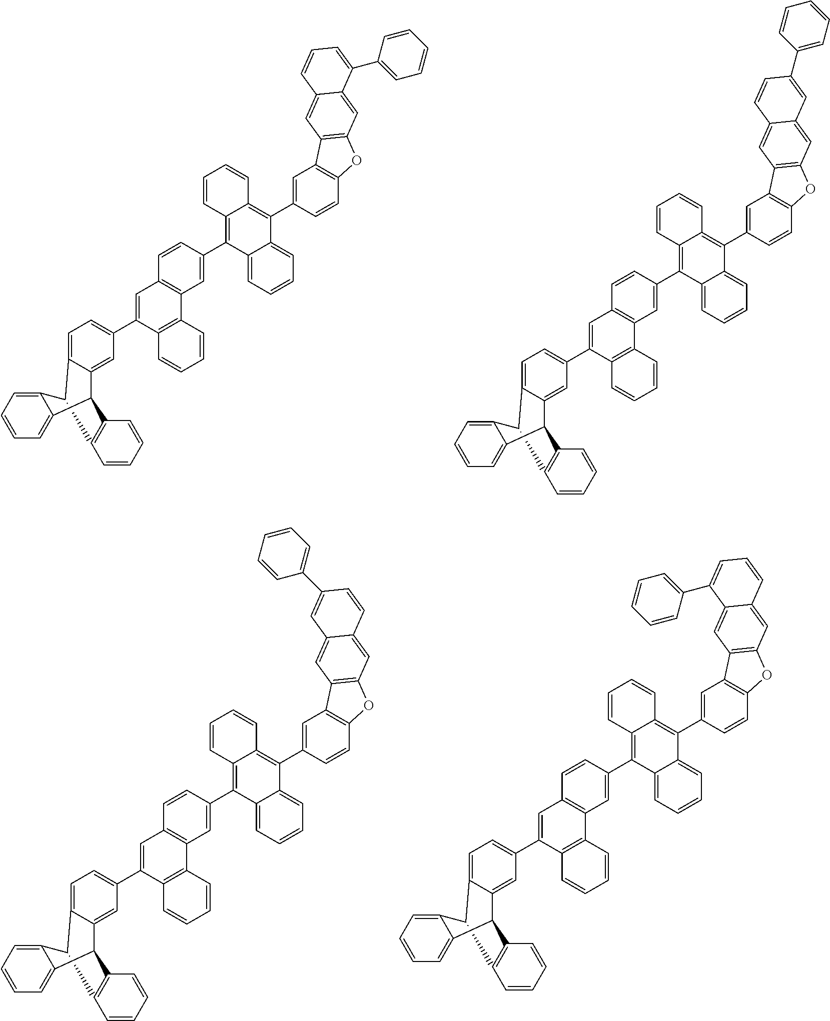

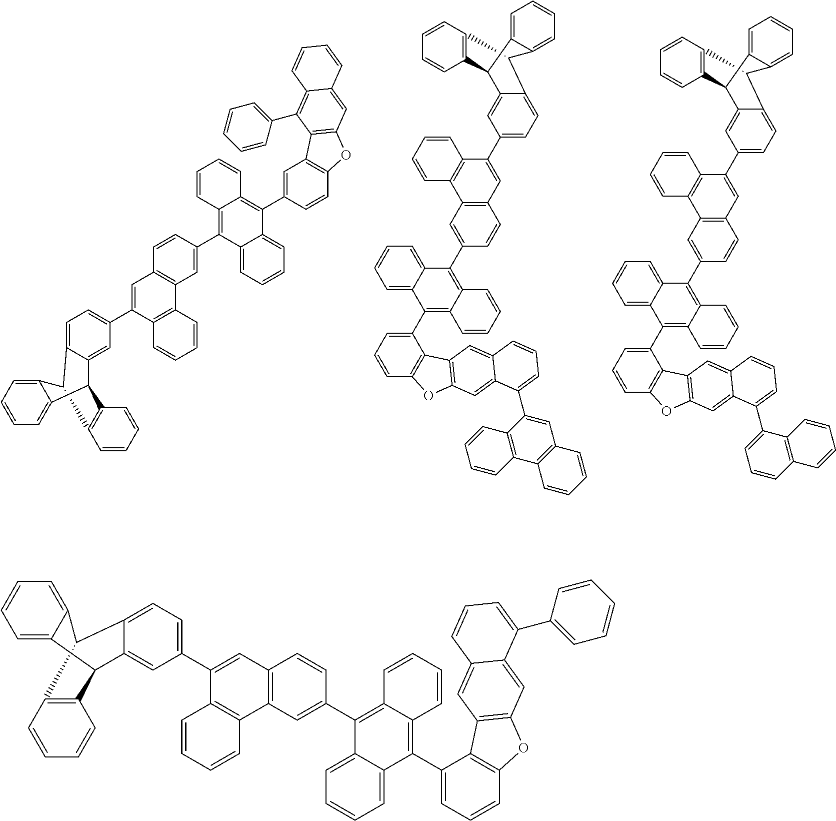

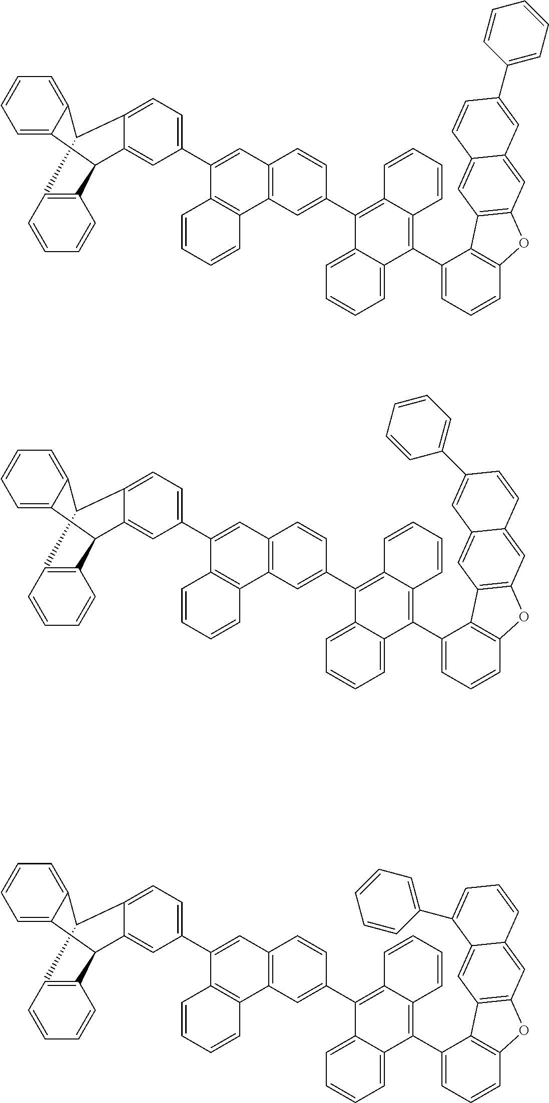 [SMILES: c1ccc(-c2ccc3cc4c(cc3c2)oc2cccc(-c3c5ccccc5c(-c5ccc6cc(-c7ccc8c(c7)[C@H]7c9ccccc9[C@@H]8c8ccccc87)c7ccccc7c6c5)c5ccccc35)c24)cc1 ; c1ccc(-c2ccc3cc4oc5cccc(-c6c7ccccc7c(-c7ccc8cc(-c9ccc%10c(c9)[C@H]9c%11ccccc%11[C@@H]%10c%10ccccc%109)c9ccccc9c8c7)c7ccccc67)c5c4cc3c2)cc1 ; c1ccc(-c2cccc3cc4oc5cccc(-c6c7ccccc7c(-c7ccc8cc(-c9ccc%10c(c9)[C@H]9c%11ccccc%11[C@@H]%10c%10ccccc%109)c9ccccc9c8c7)c7ccccc67)c5c4cc23)cc1]

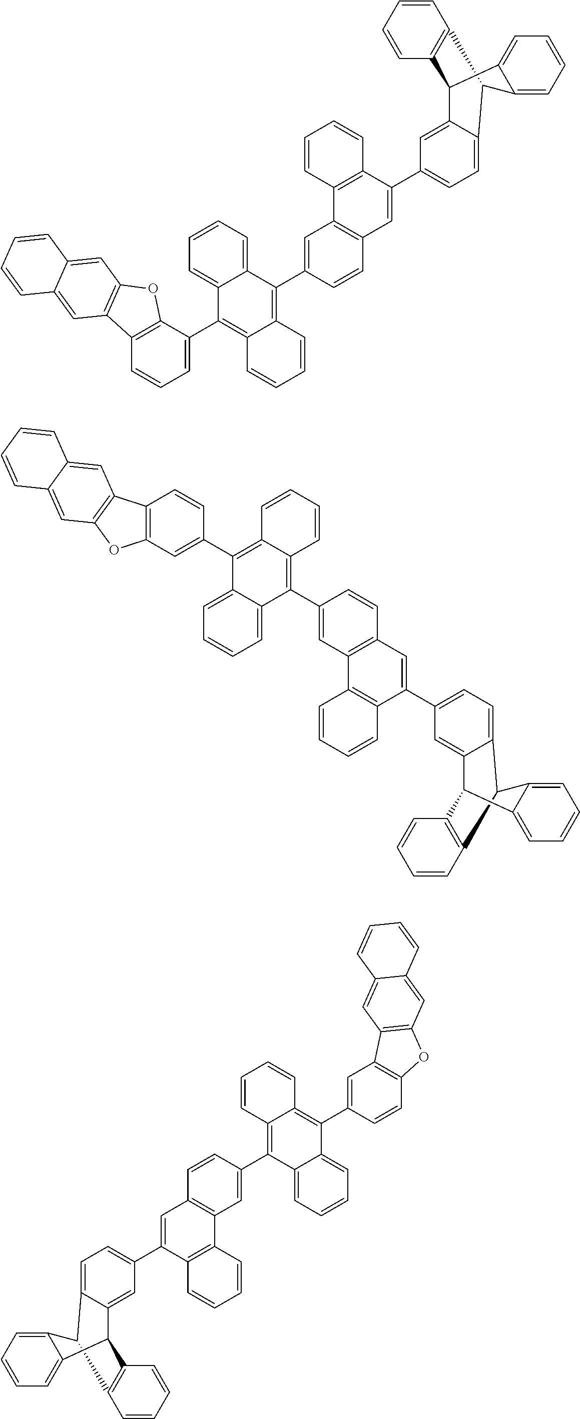 [SMILES: c1ccc2cc3c(cc2c1)oc1c(-c2c4ccccc4c(-c4ccc5cc(-c6ccc7c(c6)[C@H]6c8ccccc8[C@@H]7c7ccccc76)c6ccccc6c5c4)c4ccccc24)cccc13 ; c1ccc2cc3c(cc2c1)oc1cc(-c2c4ccccc4c(-c4ccc5cc(-c6ccc7c(c6)[C@H]6c8ccccc8[C@@H]7c7ccccc76)c6ccccc6c5c4)c4ccccc24)ccc13 ; c1ccc2cc3c(cc2c1)oc1ccc(-c2c4ccccc4c(-c4ccc5cc(-c6ccc7c(c6)[C@H]6c8ccccc8[C@@H]7c7ccccc76)c6ccccc6c5c4)c4ccccc24)cc13]

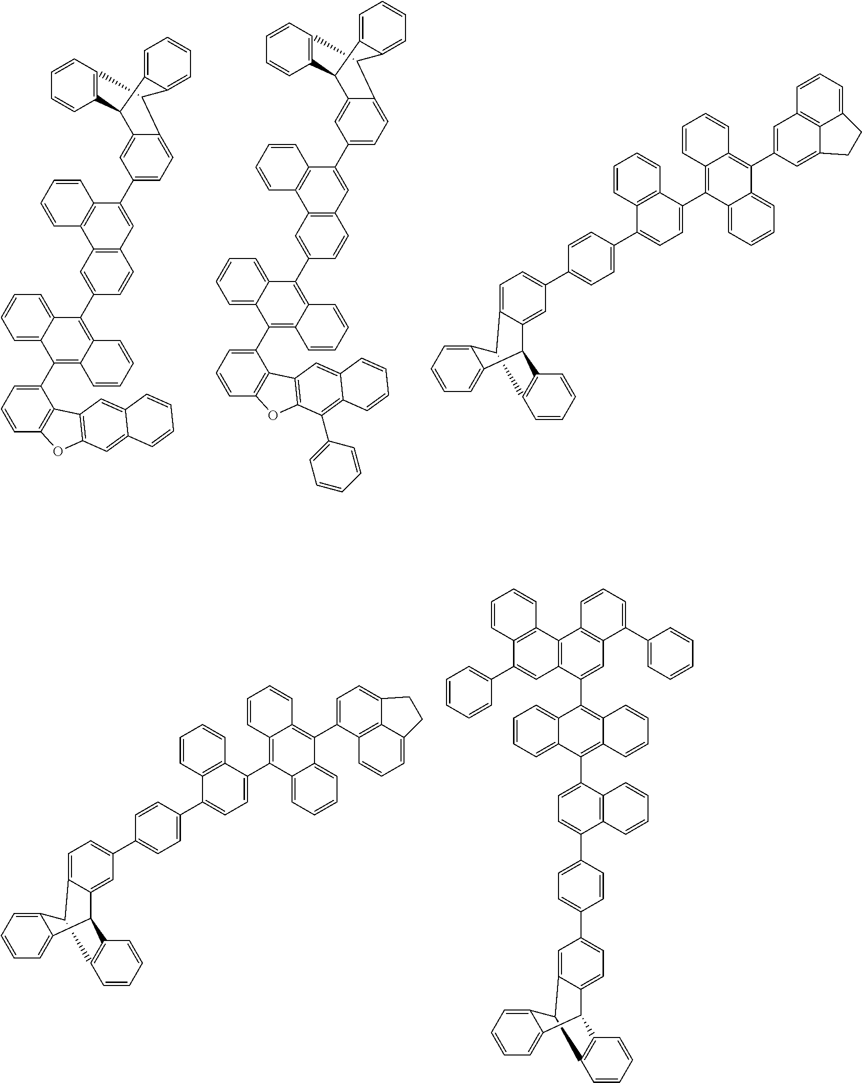

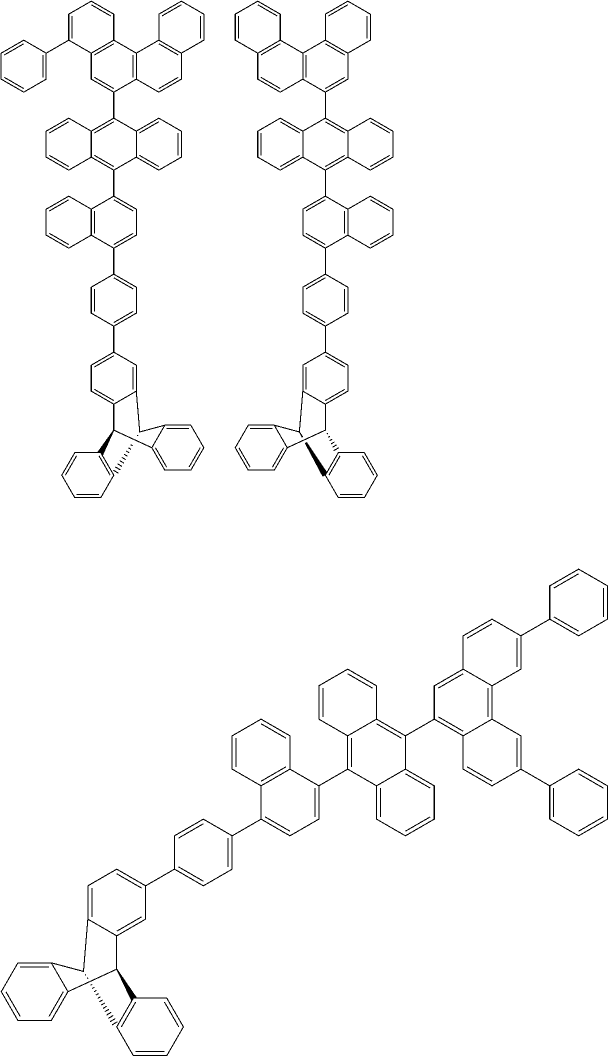

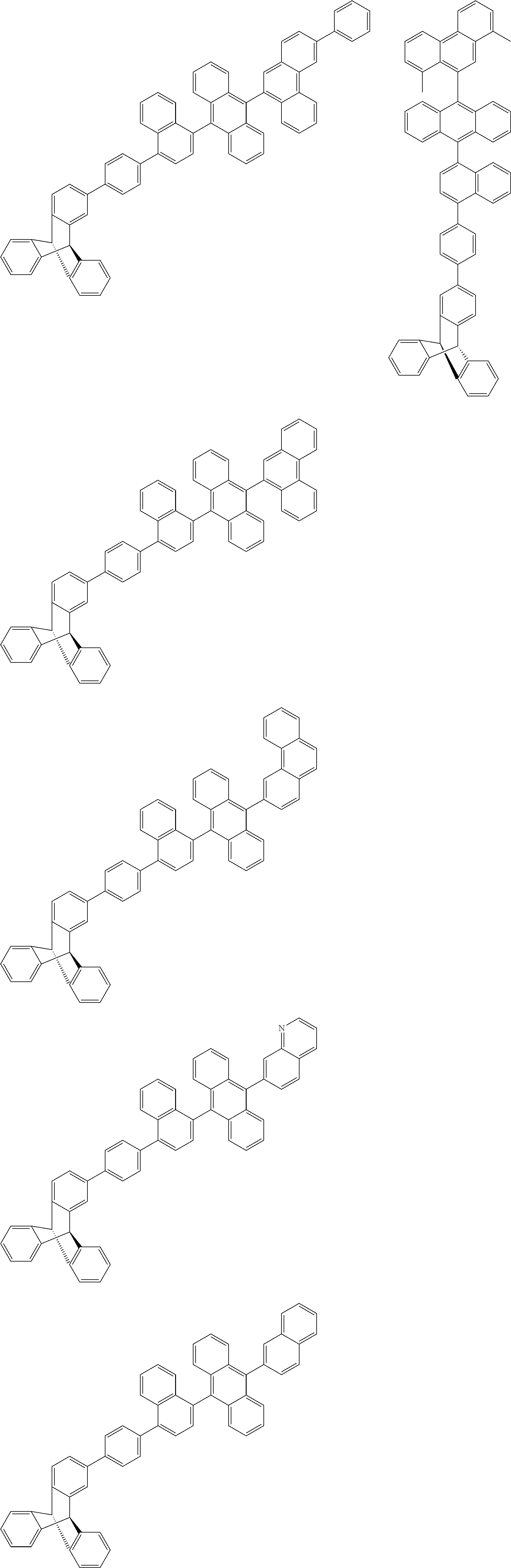

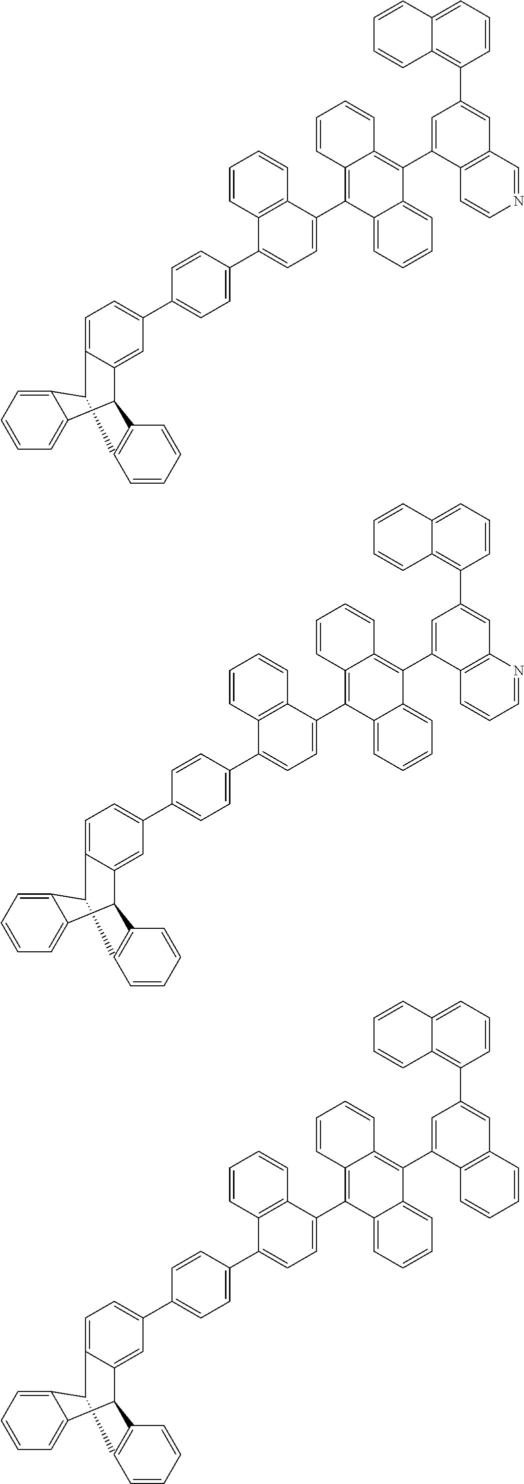

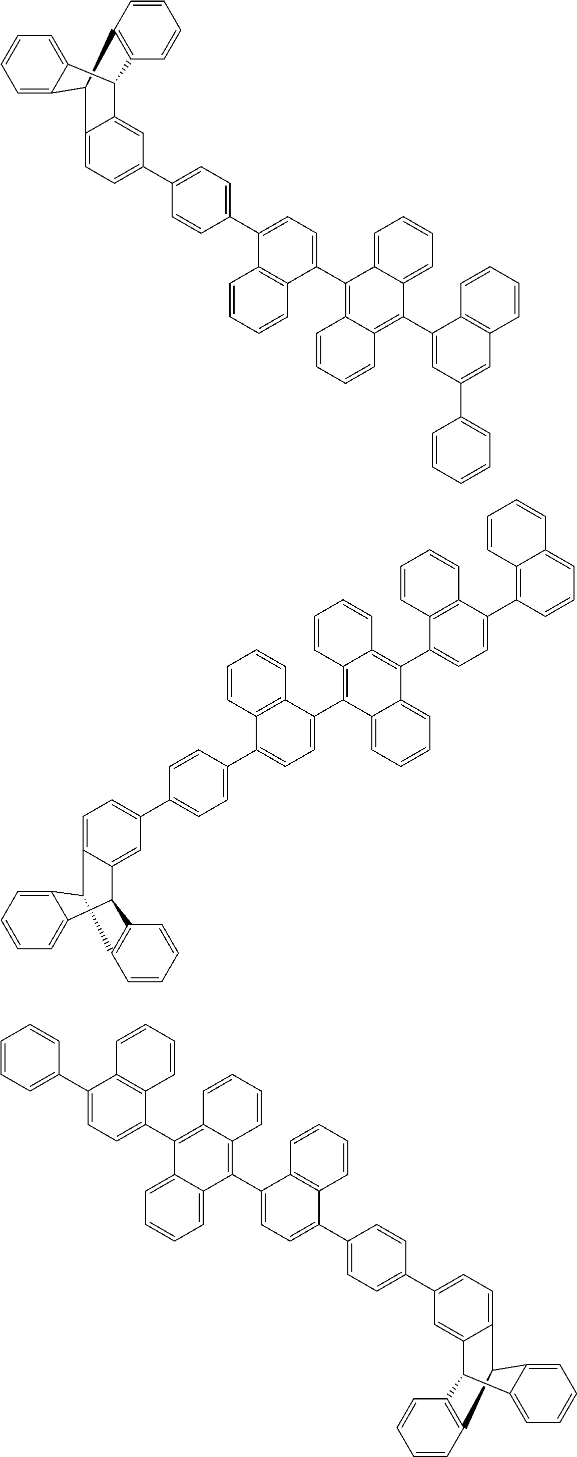

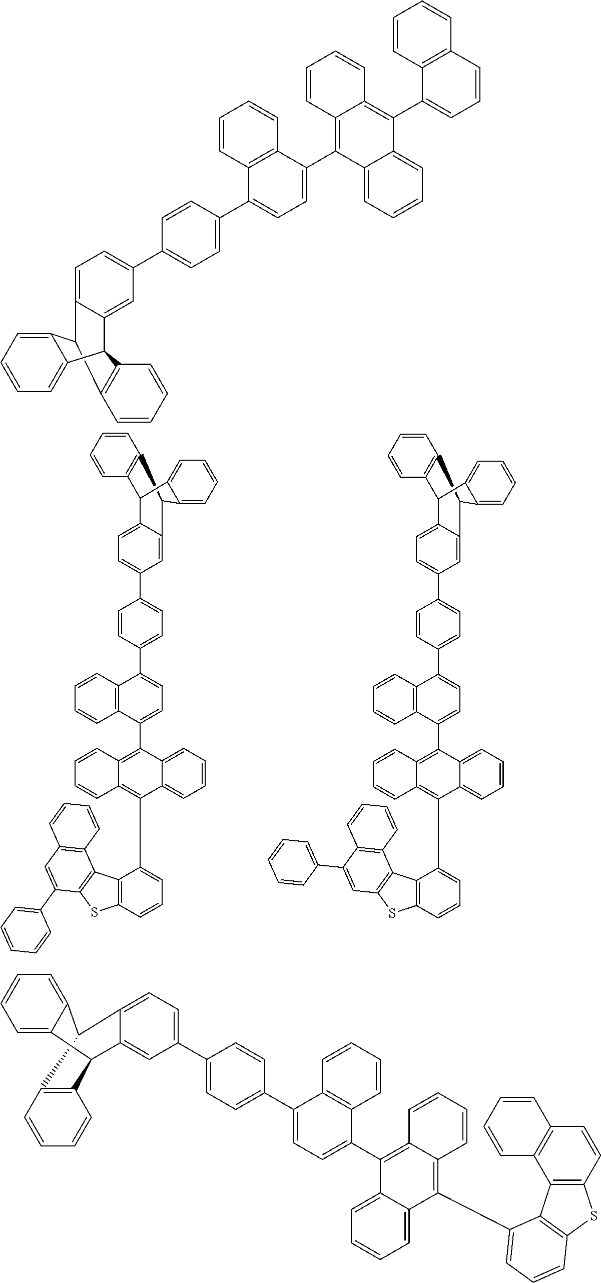

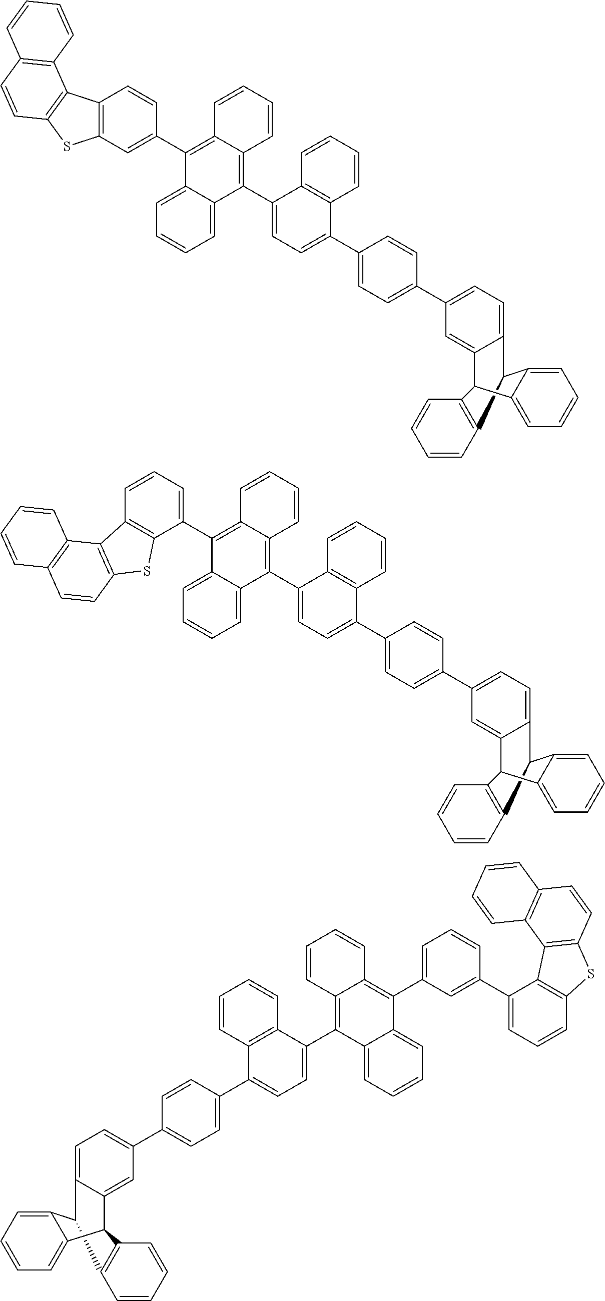

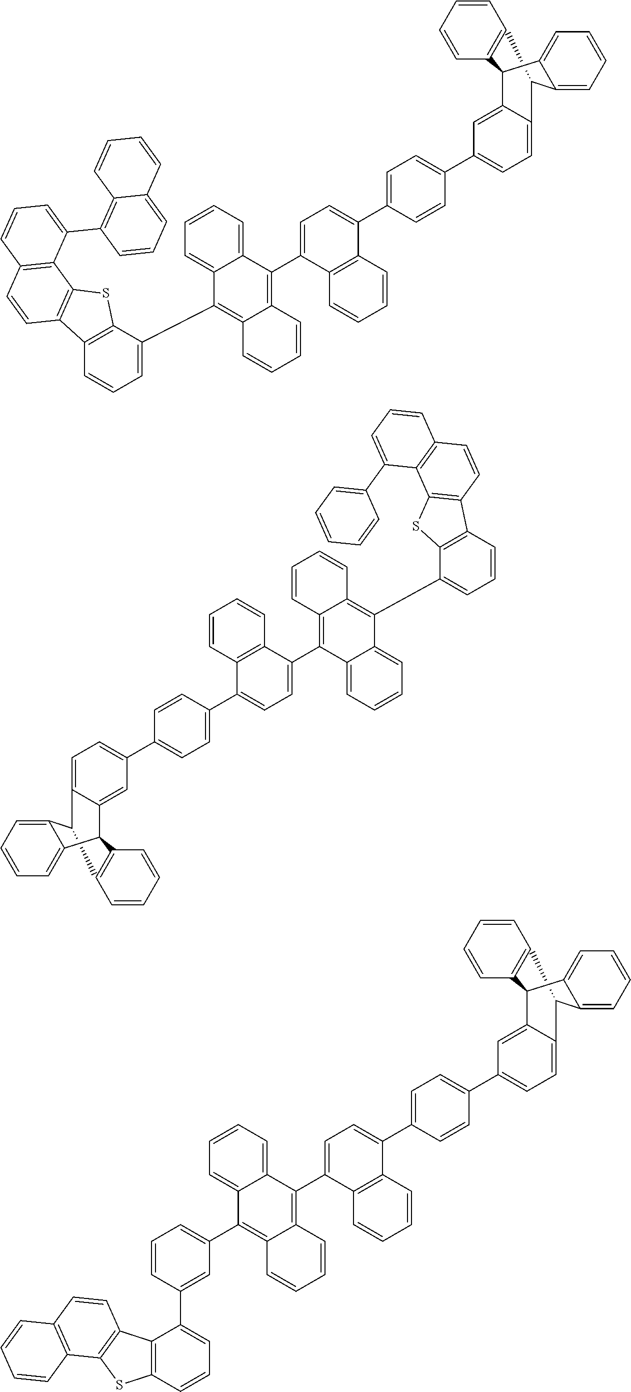 [SMILES: c1ccc2c(-c3cccc4ccc5c6cccc(-c7c8ccccc8c(-c8ccc(-c9ccc(-c%10ccc%11c(c%10)[C@H]%10c%12ccccc%12[C@@H]%11c%11ccccc%11%10)cc9)c9ccccc89)c8ccccc78)c6sc5c34)cccc2c1 ; c1ccc(-c2cccc3ccc4c5cccc(-c6c7ccccc7c(-c7ccc(-c8ccc(-c9ccc%10c(c9)[C@H]9c%11ccccc%11[C@@H]%10c%10ccccc%109)cc8)c8ccccc78)c7ccccc67)c5sc4c23)cc1 ; c1cc(-c2c3ccccc3c(-c3ccc(-c4ccc(-c5ccc6c(c5)[C@H]5c7ccccc7[C@@H]6c6ccccc65)cc4)c4ccccc34)c3ccccc23)cc(-c2cccc3sc4c5ccccc5ccc4c23)c1]

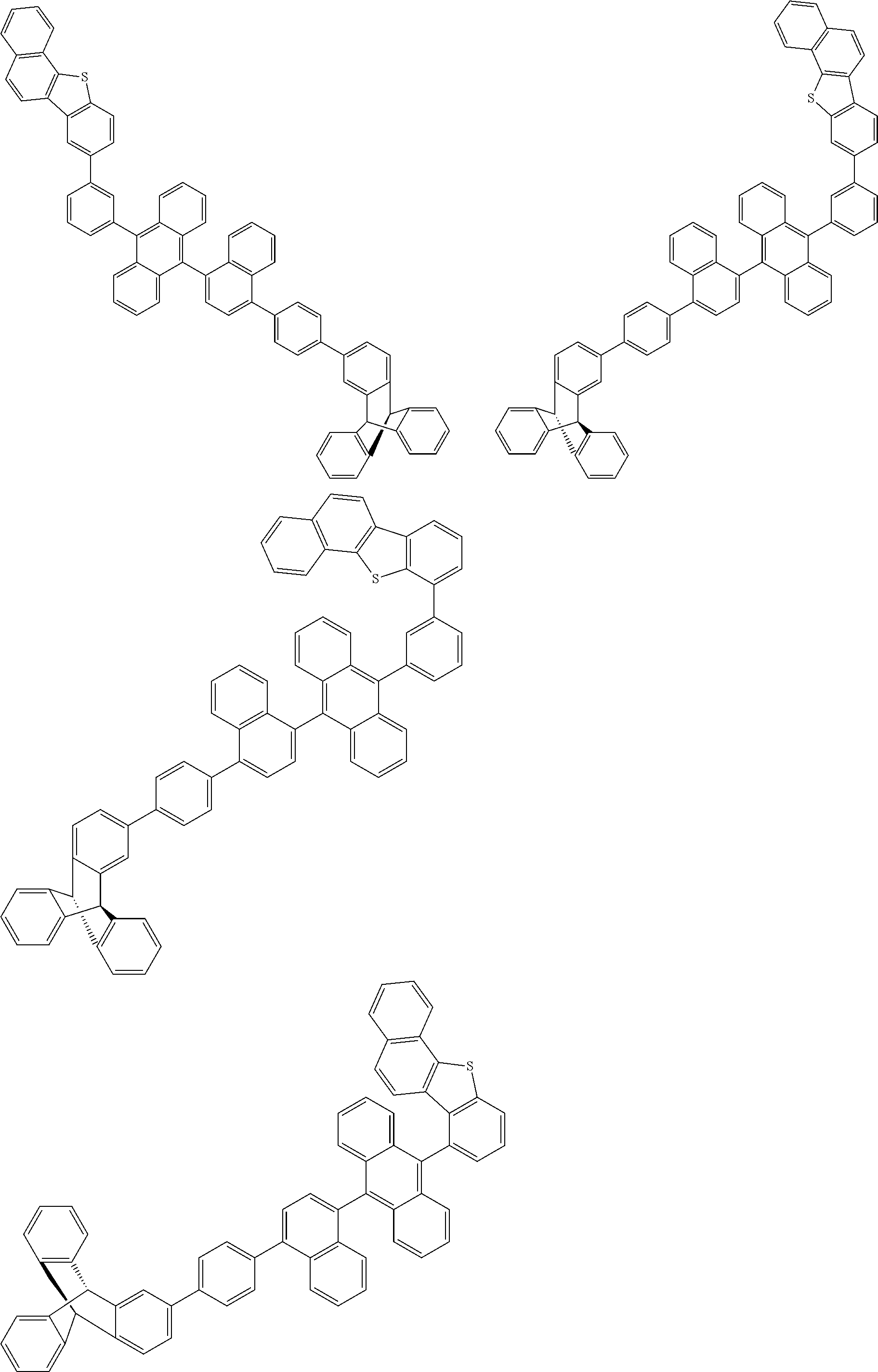

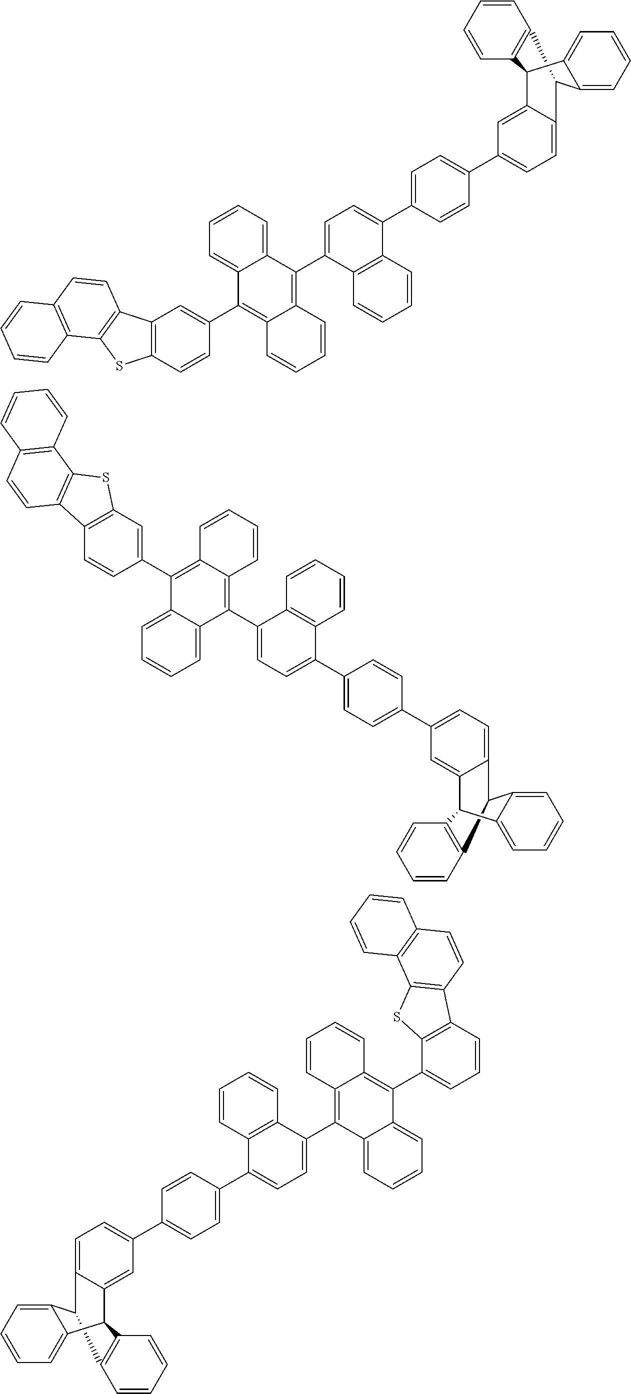

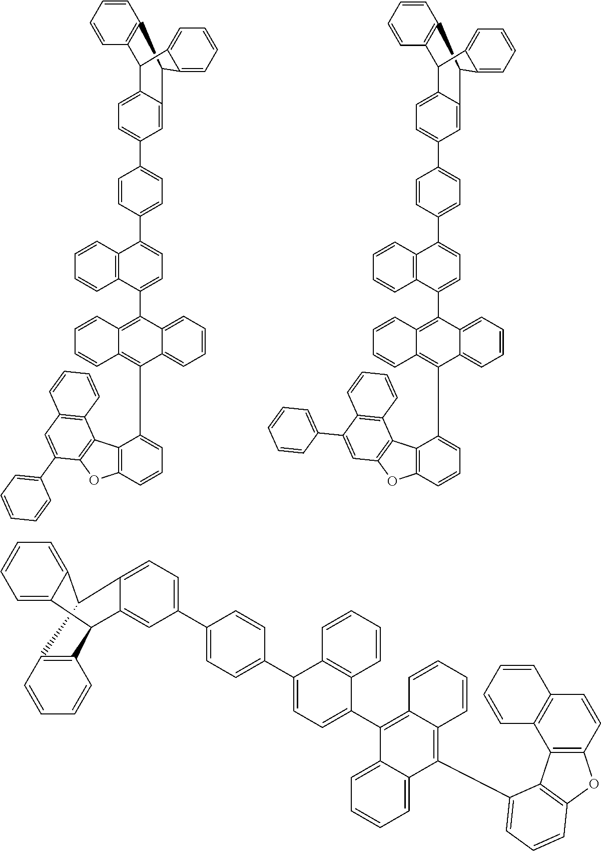

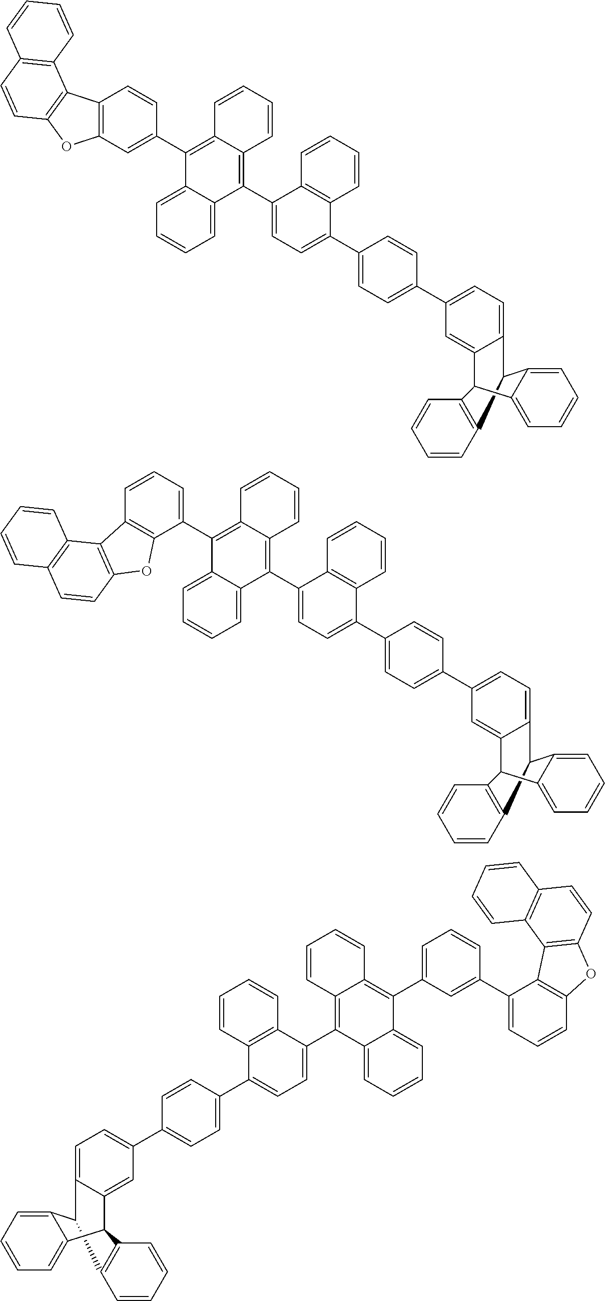

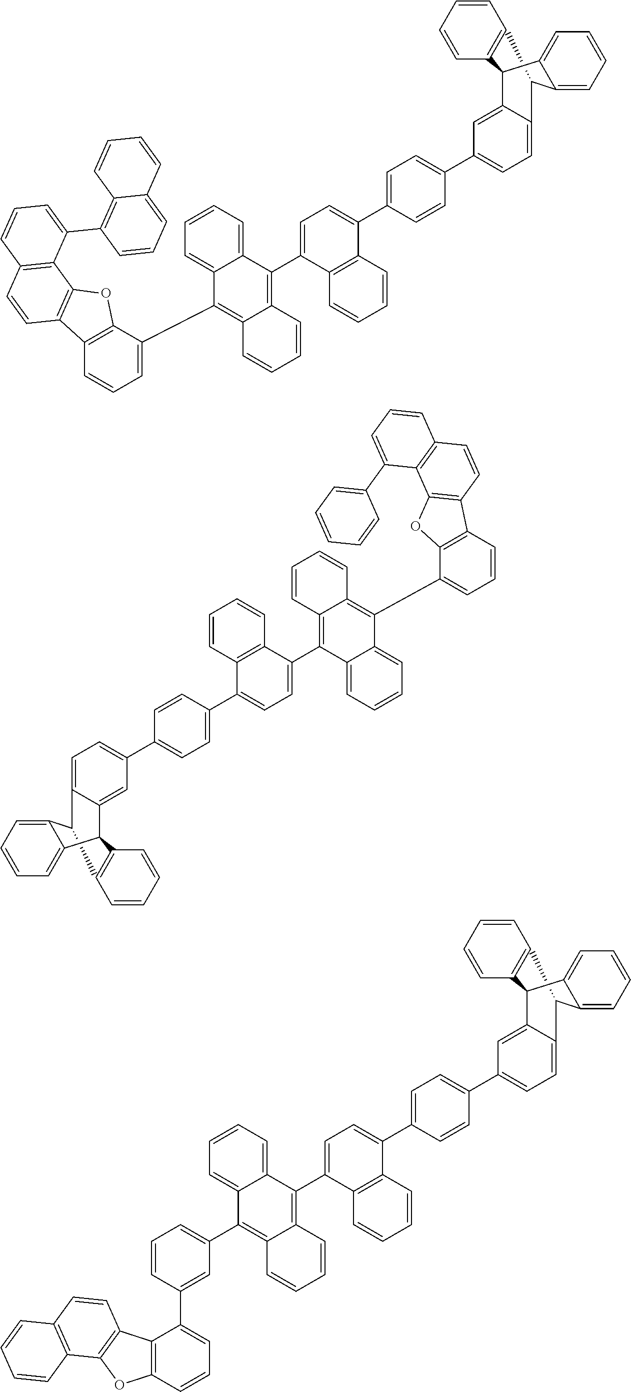 [SMILES: c1ccc2c(-c3cccc4ccc5c6cccc(-c7c8ccccc8c(-c8ccc(-c9ccc(-c%10ccc%11c(c%10)[C@H]%10c%12ccccc%12[C@@H]%11c%11ccccc%11%10)cc9)c9ccccc89)c8ccccc78)c6oc5c34)cccc2c1 ; c1ccc(-c2cccc3ccc4c5cccc(-c6c7ccccc7c(-c7ccc(-c8ccc(-c9ccc%10c(c9)[C@H]9c%11ccccc%11[C@@H]%10c%10ccccc%109)cc8)c8ccccc78)c7ccccc67)c5oc4c23)cc1 ; c1cc(-c2c3ccccc3c(-c3ccc(-c4ccc(-c5ccc6c(c5)[C@H]5c7ccccc7[C@@H]6c6ccccc65)cc4)c4ccccc34)c3ccccc23)cc(-c2cccc3oc4c5ccccc5ccc4c23)c1]

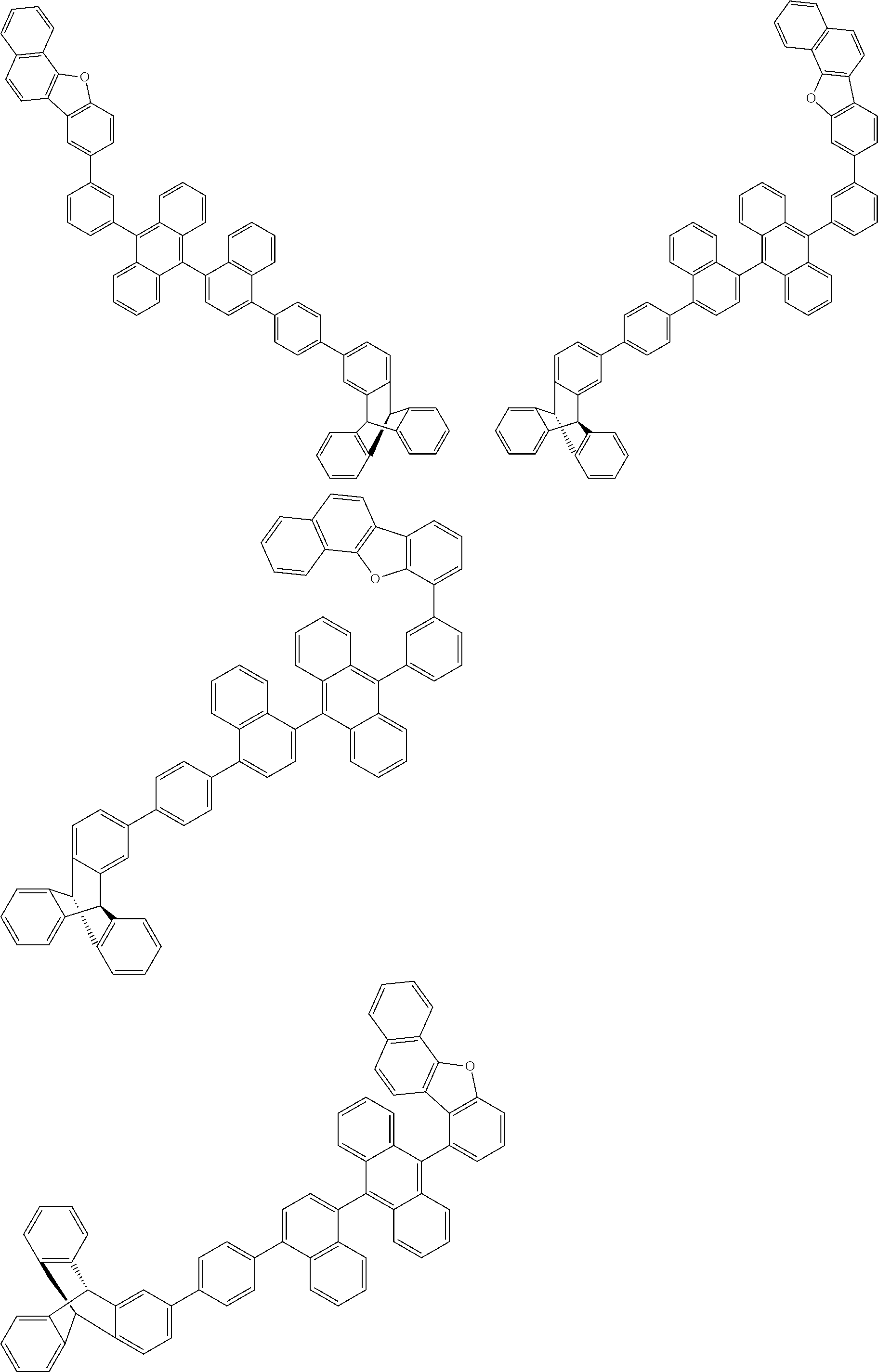

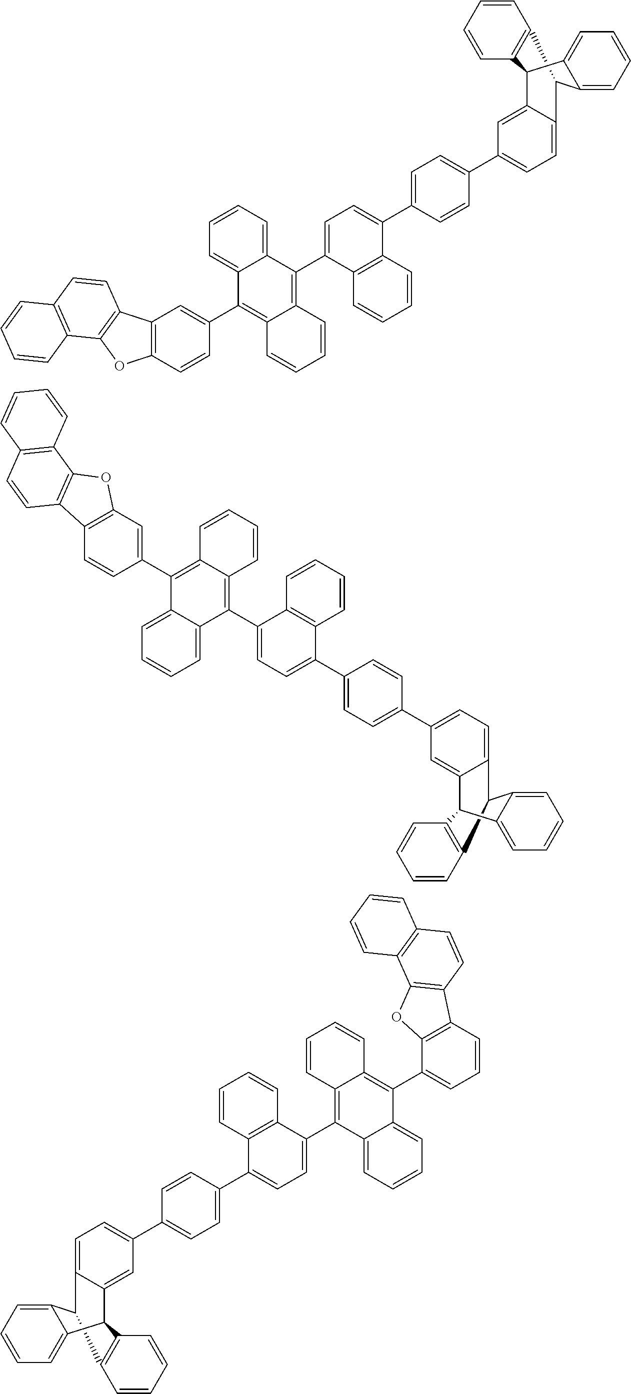

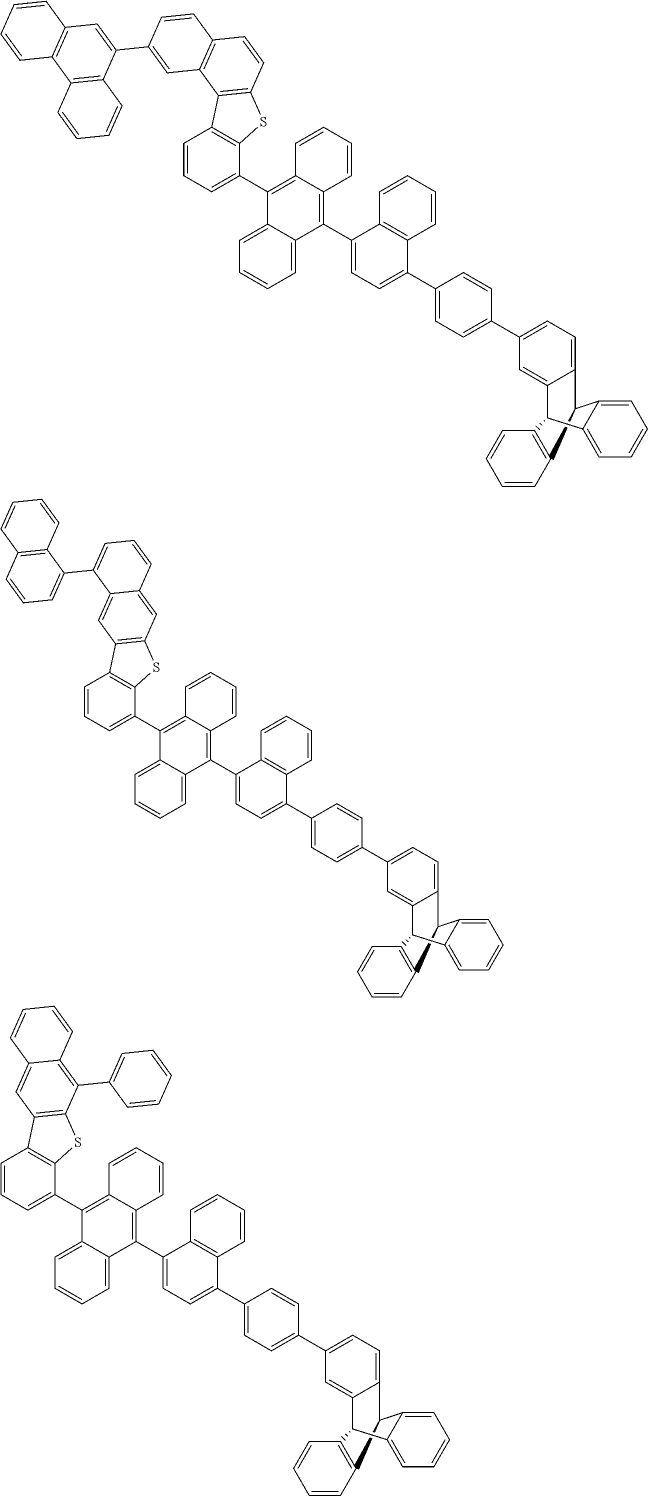 [SMILES: c1ccc2c(c1)cc(-c1ccc3ccc4sc5c(-c6c7ccccc7c(-c7ccc(-c8ccc(-c9ccc%10c(c9)[C@H]9c%11ccccc%11[C@@H]%10c%10ccccc%109)cc8)c8ccccc78)c7ccccc67)cccc5c4c3c1)c1ccccc12 ; c1ccc2c(-c3cccc4cc5sc6c(-c7c8ccccc8c(-c8ccc(-c9ccc(-c%10ccc%11c(c%10)[C@H]%10c%12ccccc%12[C@@H]%11c%11ccccc%11%10)cc9)c9ccccc89)c8ccccc78)cccc6c5cc34)cccc2c1 ; c1ccc(-c2c3ccccc3cc3c2sc2c(-c4c5ccccc5c(-c5ccc(-c6ccc(-c7ccc8c(c7)[C@H]7c9ccccc9[C@@H]8c8ccccc87)cc6)c6ccccc56)c5ccccc45)cccc23)cc1]

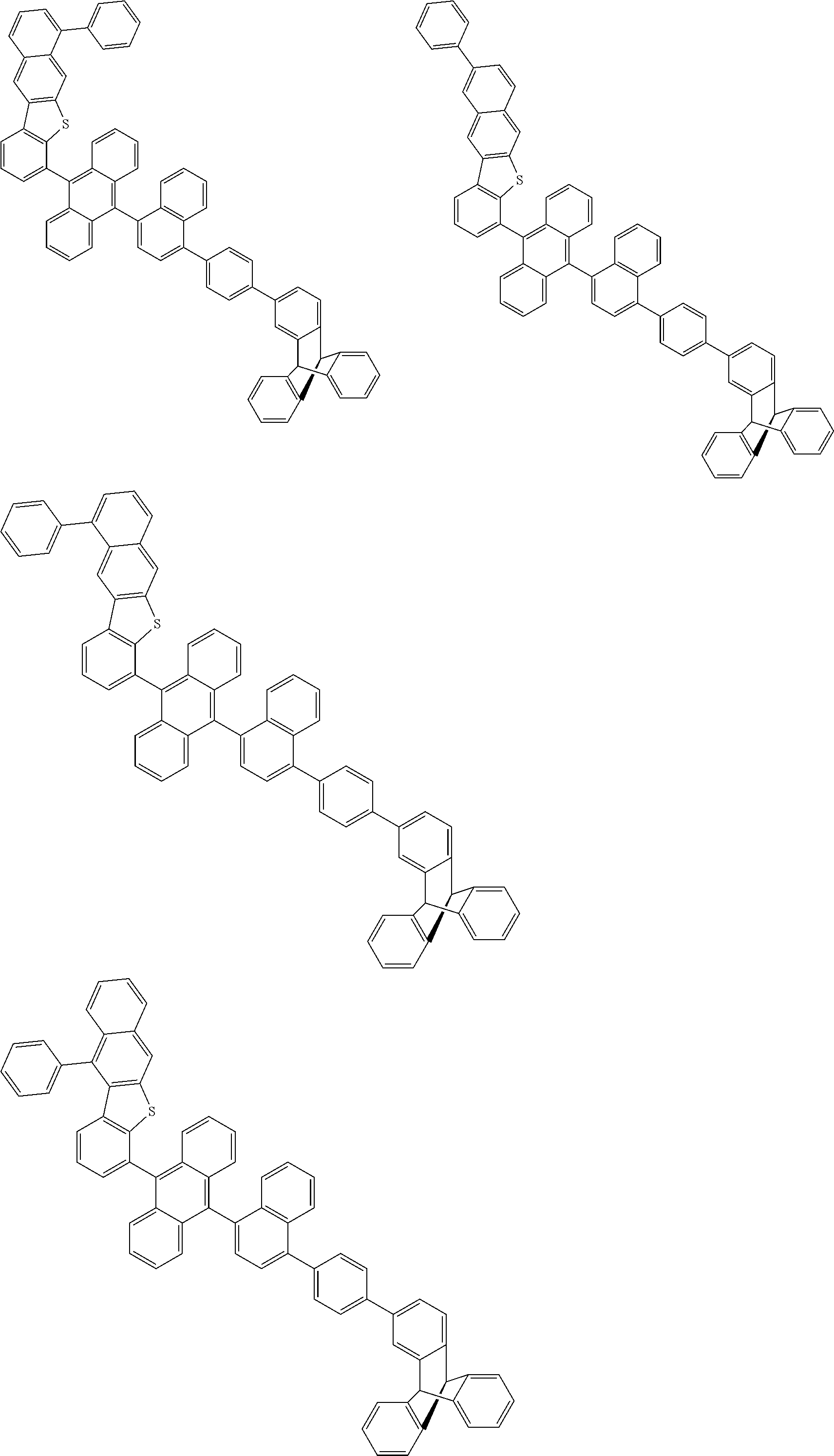

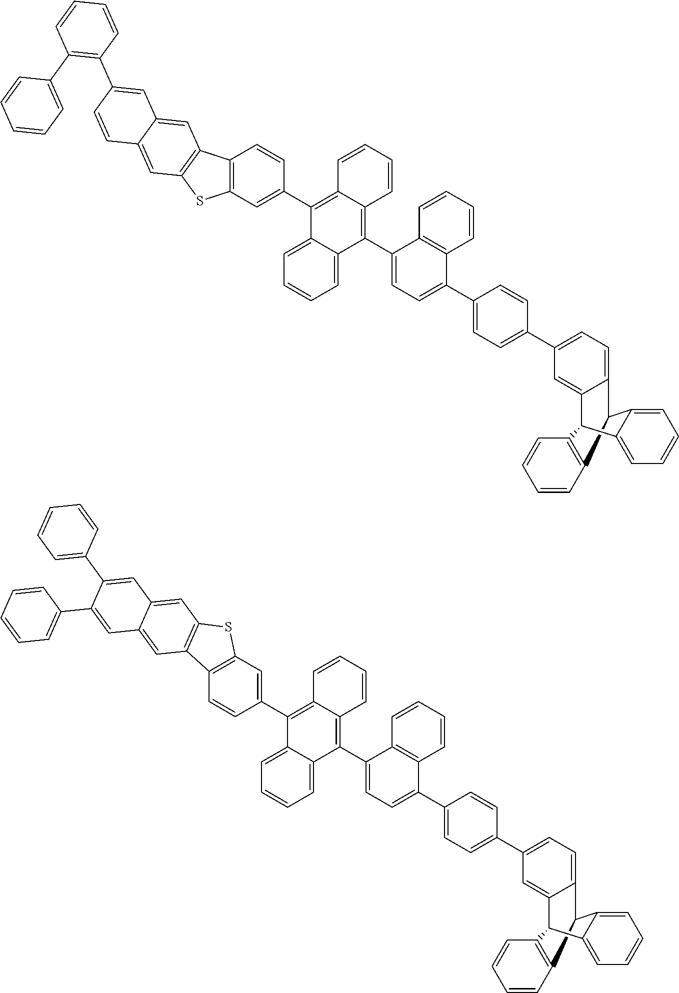 [SMILES: c1ccc(-c2ccccc2-c2ccc3cc4sc5cc(-c6c7ccccc7c(-c7ccc(-c8ccc(-c9ccc%10c(c9)[C@H]9c%11ccccc%11[C@@H]%10c%10ccccc%109)cc8)c8ccccc78)c7ccccc67)ccc5c4cc3c2)cc1 ; c1ccc(-c2cc3cc4sc5cc(-c6c7ccccc7c(-c7ccc(-c8ccc(-c9ccc%10c(c9)[C@H]9c%11ccccc%11[C@@H]%10c%10ccccc%109)cc8)c8ccccc78)c7ccccc67)ccc5c4cc3cc2-c2ccccc2)cc1]

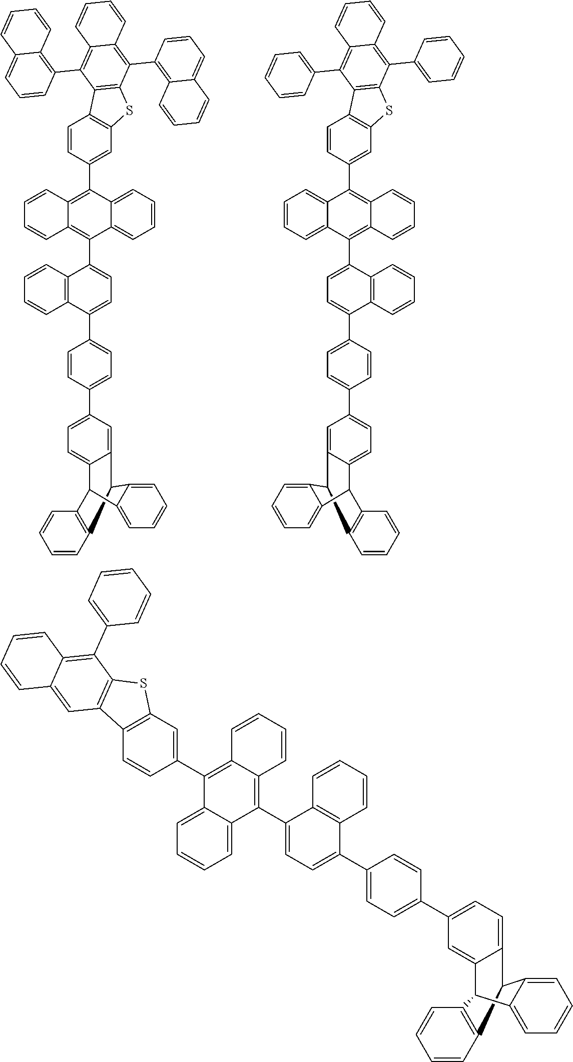

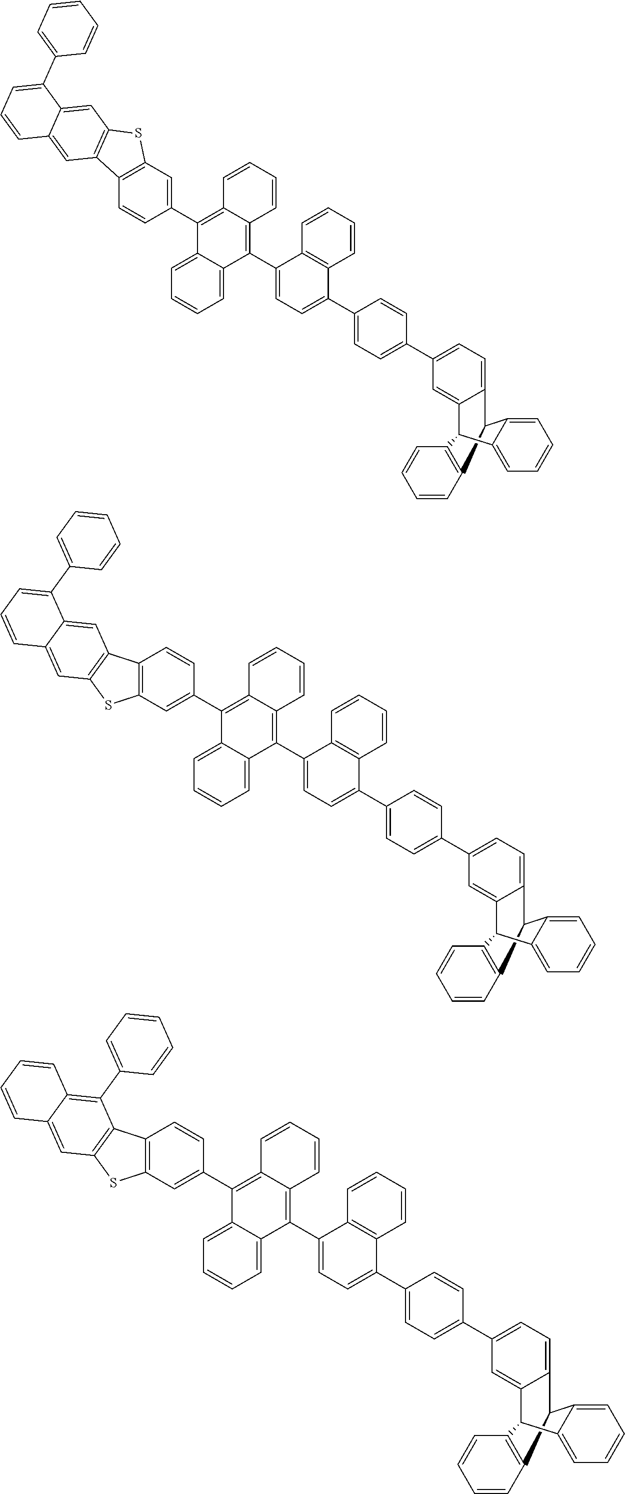 [SMILES: c1ccc(-c2cccc3cc4c(cc23)sc2cc(-c3c5ccccc5c(-c5ccc(-c6ccc(-c7ccc8c(c7)[C@H]7c9ccccc9[C@@H]8c8ccccc87)cc6)c6ccccc56)c5ccccc35)ccc24)cc1 ; c1ccc(-c2cccc3cc4sc5cc(-c6c7ccccc7c(-c7ccc(-c8ccc(-c9ccc%10c(c9)[C@H]9c%11ccccc%11[C@@H]%10c%10ccccc%109)cc8)c8ccccc78)c7ccccc67)ccc5c4cc23)cc1 ; c1ccc(-c2c3ccccc3cc3sc4cc(-c5c6ccccc6c(-c6ccc(-c7ccc(-c8ccc9c(c8)[C@H]8c%10ccccc%10[C@@H]9c9ccccc98)cc7)c7ccccc67)c6ccccc56)ccc4c23)cc1]

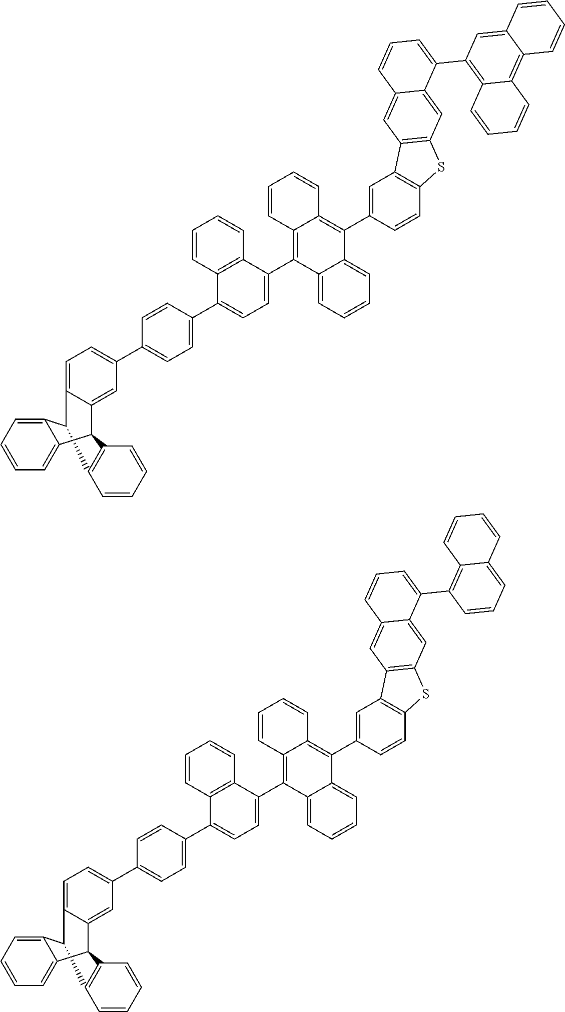 [SMILES: c1cc(-c2cc3ccccc3c3ccccc23)c2cc3sc4ccc(-c5c6ccccc6c(-c6ccc(-c7ccc(-c8ccc9c(c8)[C@H]8c%10ccccc%10[C@@H]9c9ccccc98)cc7)c7ccccc67)c6ccccc56)cc4c3cc2c1 ; c1ccc2c(-c3cccc4cc5c(cc34)sc3ccc(-c4c6ccccc6c(-c6ccc(-c7ccc(-c8ccc9c(c8)[C@H]8c%10ccccc%10[C@@H]9c9ccccc98)cc7)c7ccccc67)c6ccccc46)cc35)cccc2c1]

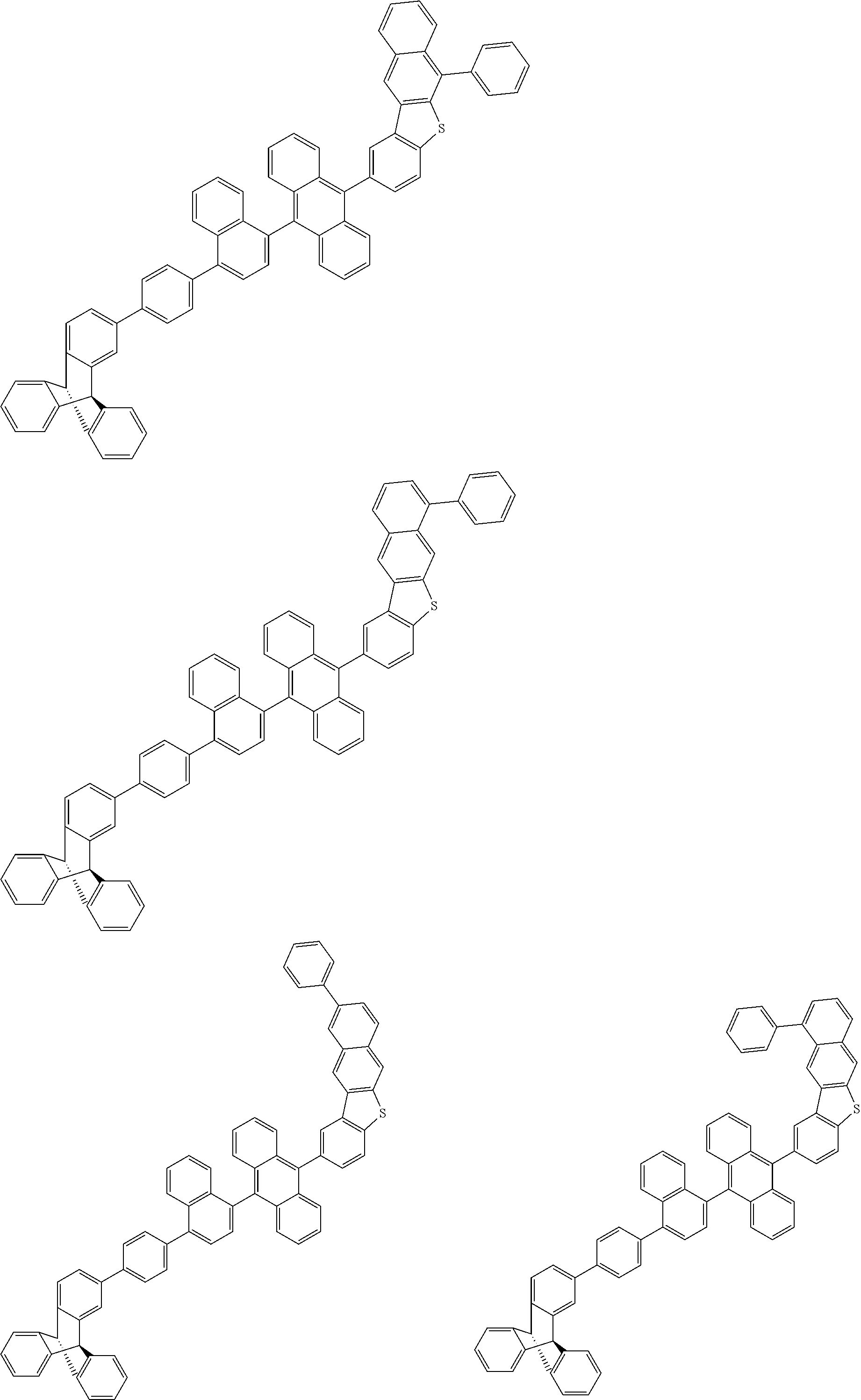

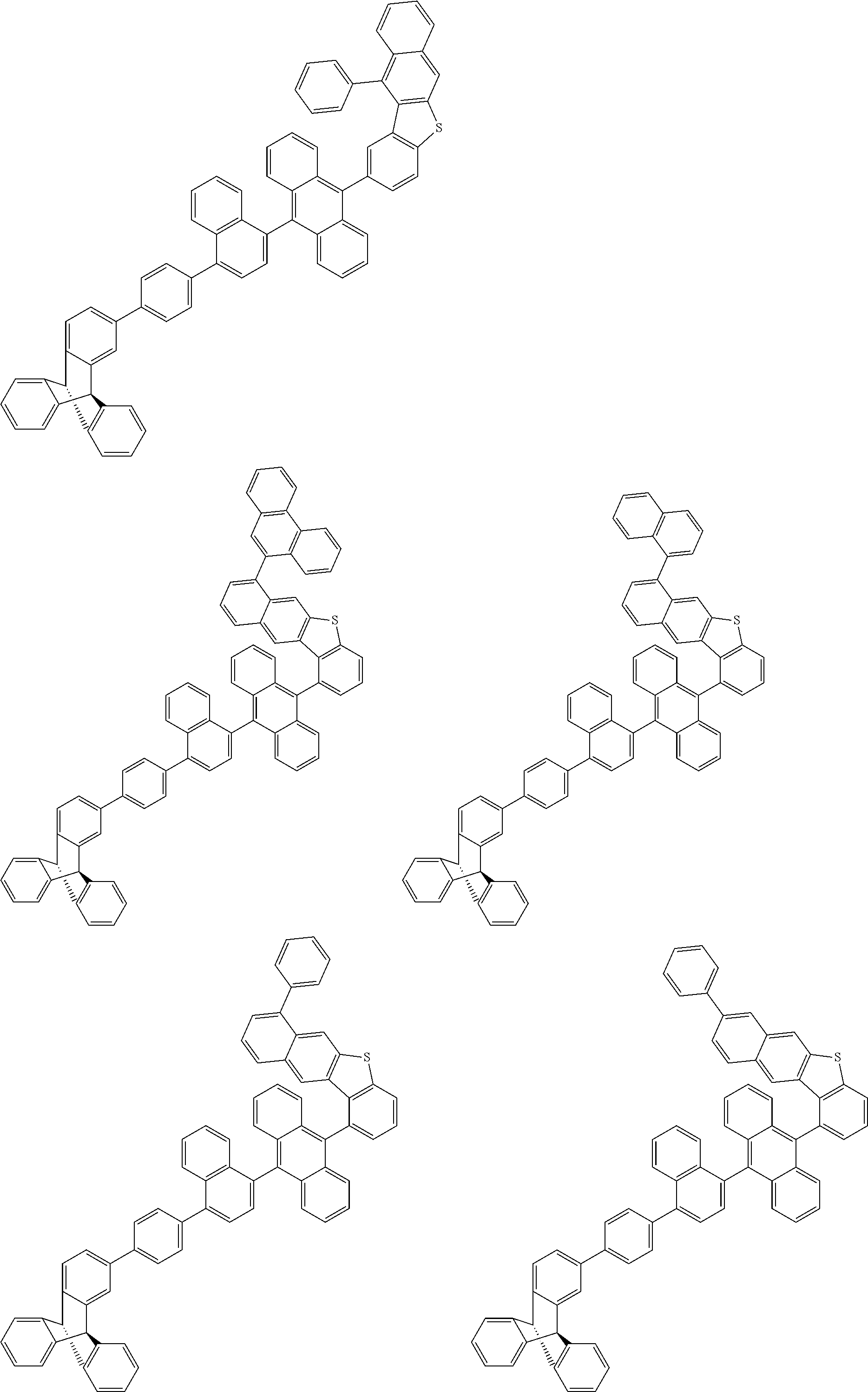

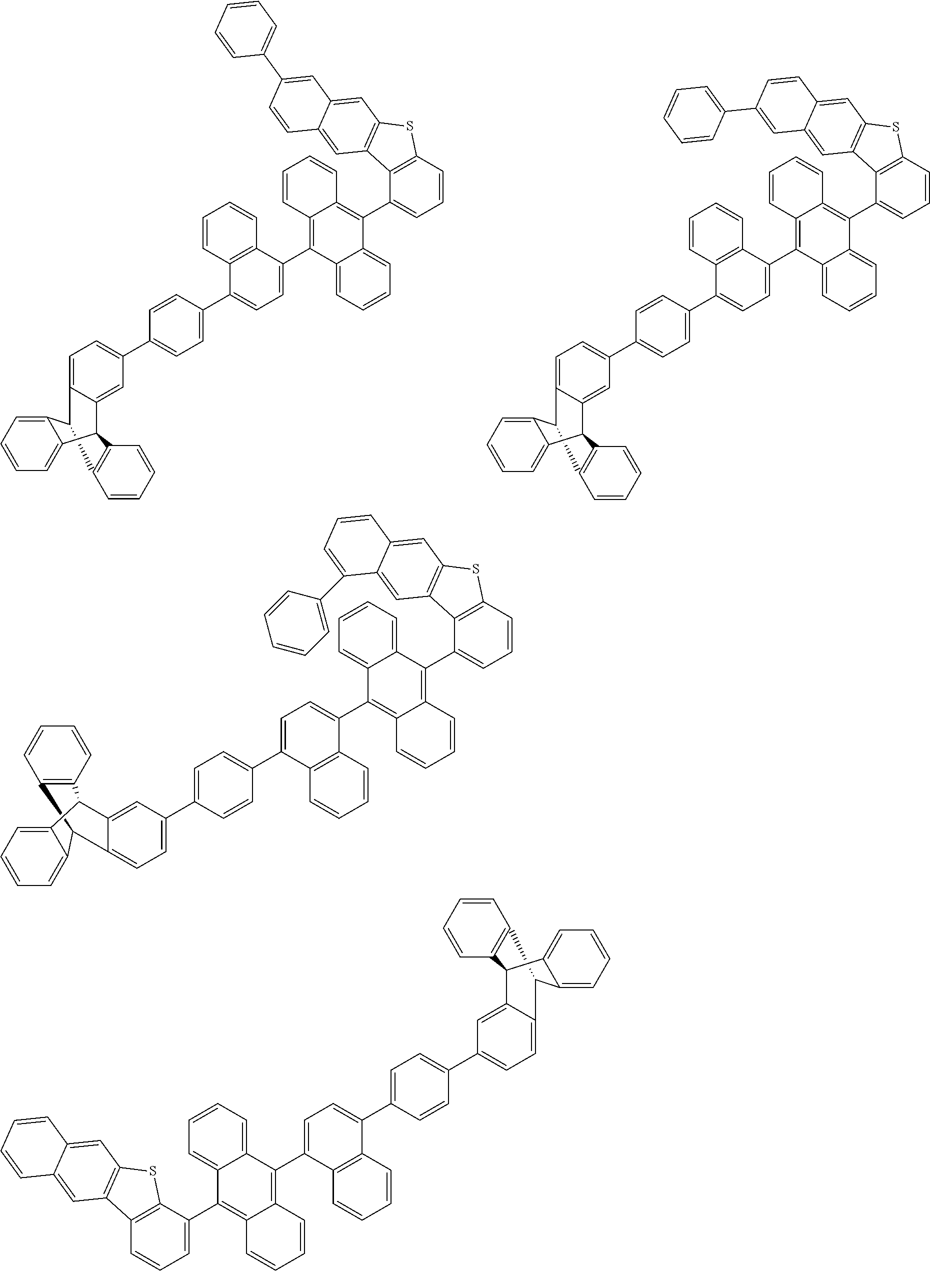

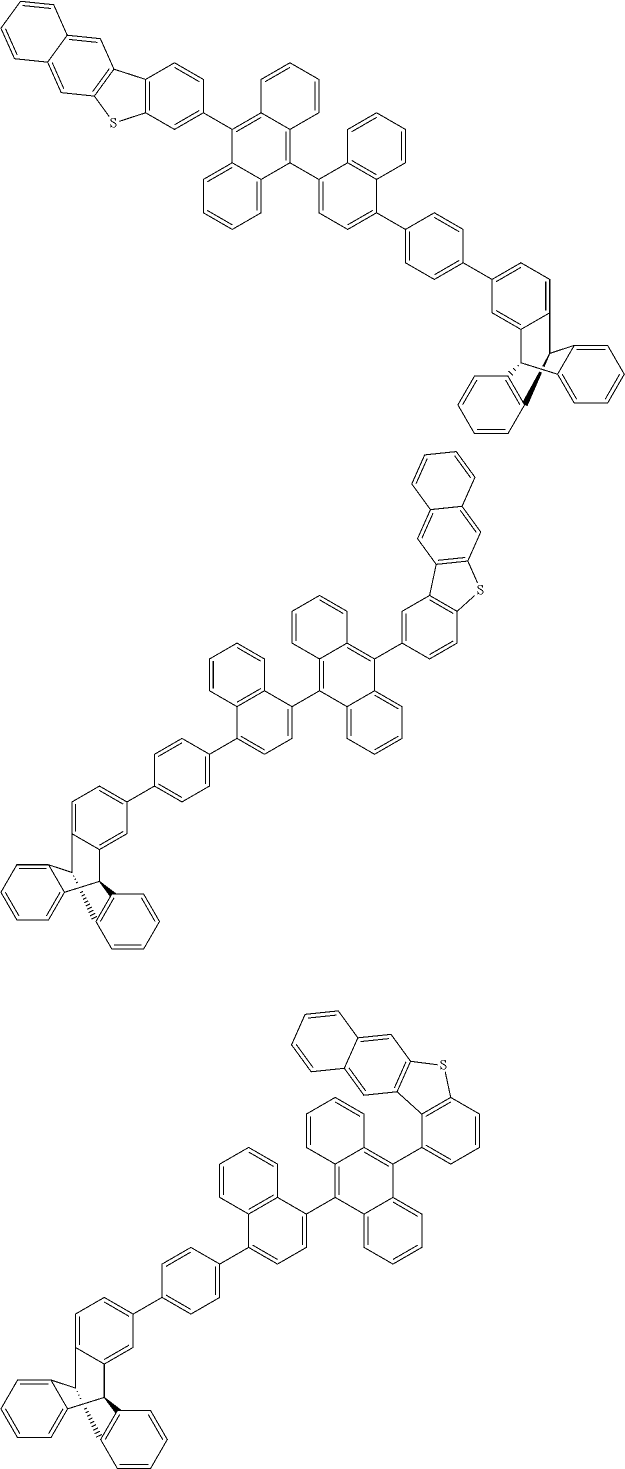

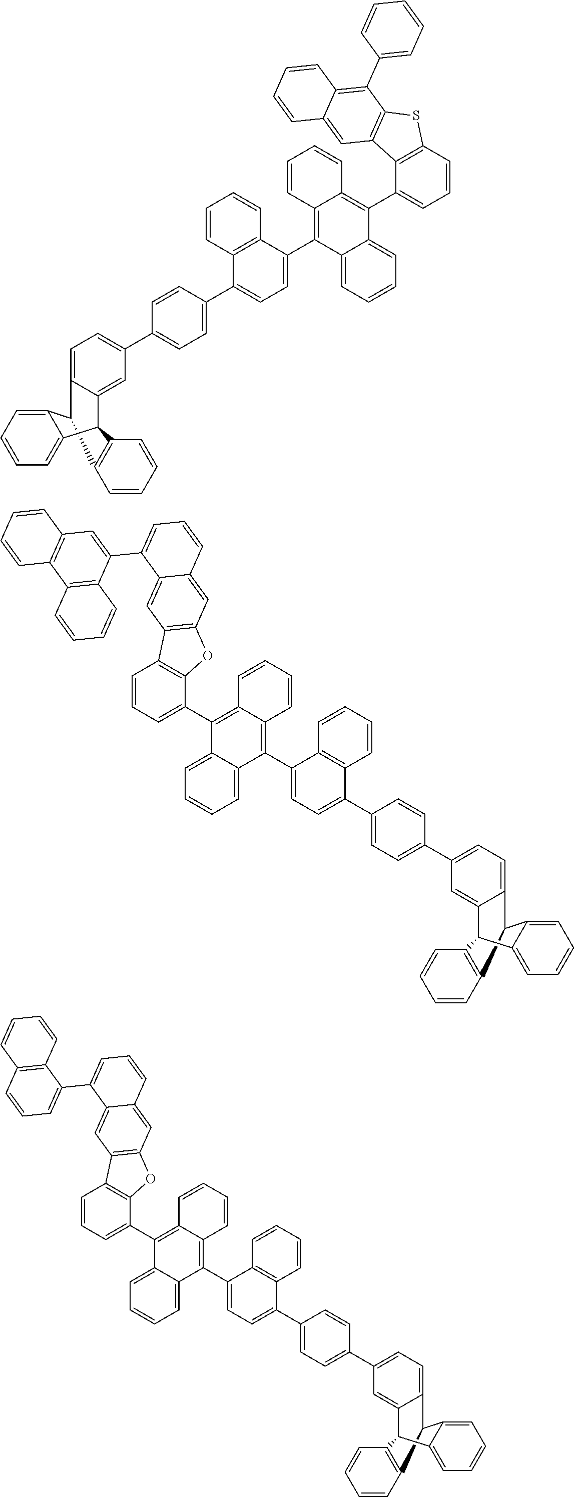

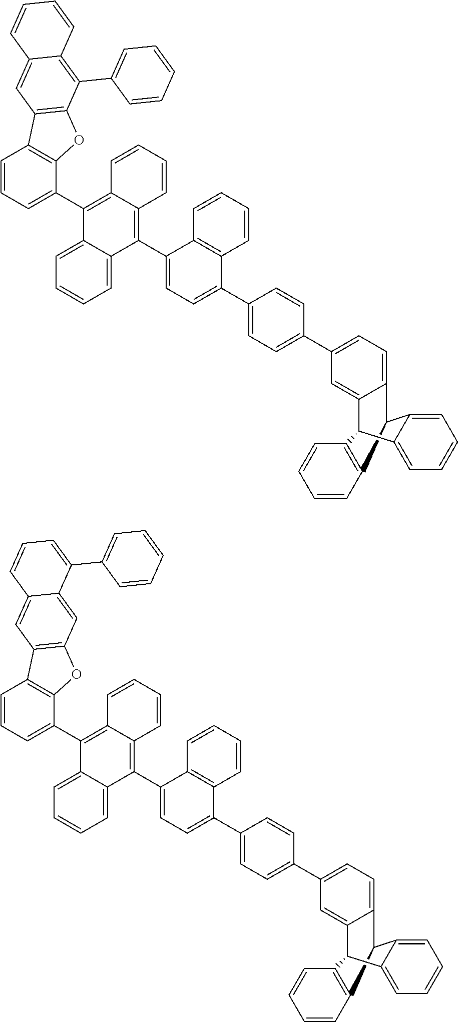 [SMILES: c1ccc(-c2c3ccccc3cc3c2oc2c(-c4c5ccccc5c(-c5ccc(-c6ccc(-c7ccc8c(c7)[C@H]7c9ccccc9[C@@H]8c8ccccc87)cc6)c6ccccc56)c5ccccc45)cccc23)cc1 ; c1ccc(-c2cccc3cc4c(cc23)oc2c(-c3c5ccccc5c(-c5ccc(-c6ccc(-c7ccc8c(c7)[C@H]7c9ccccc9[C@@H]8c8ccccc87)cc6)c6ccccc56)c5ccccc35)cccc24)cc1]

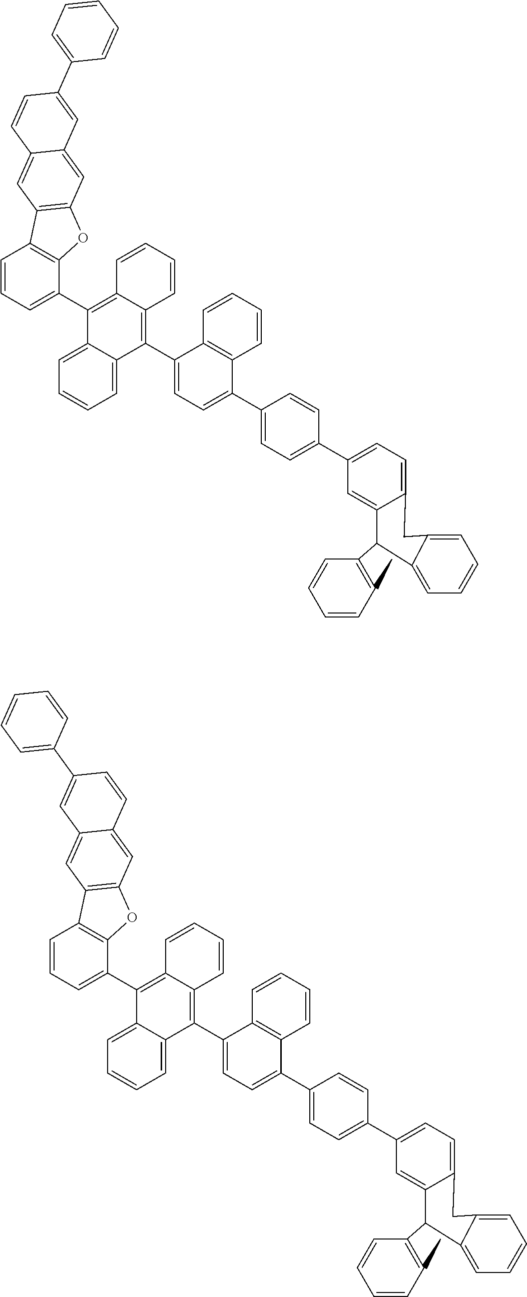

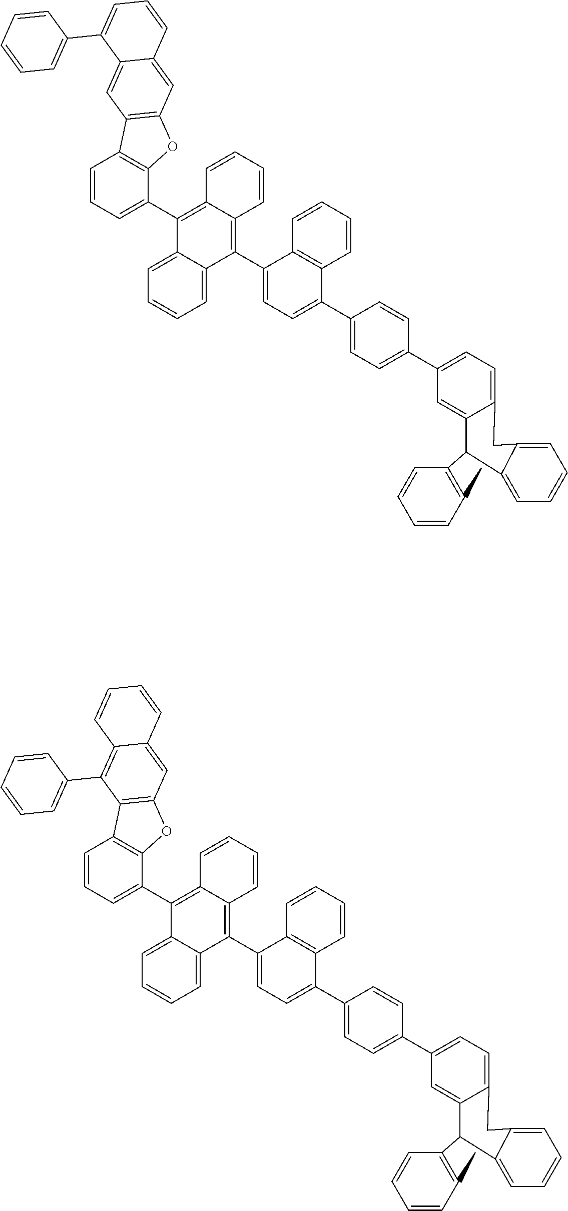

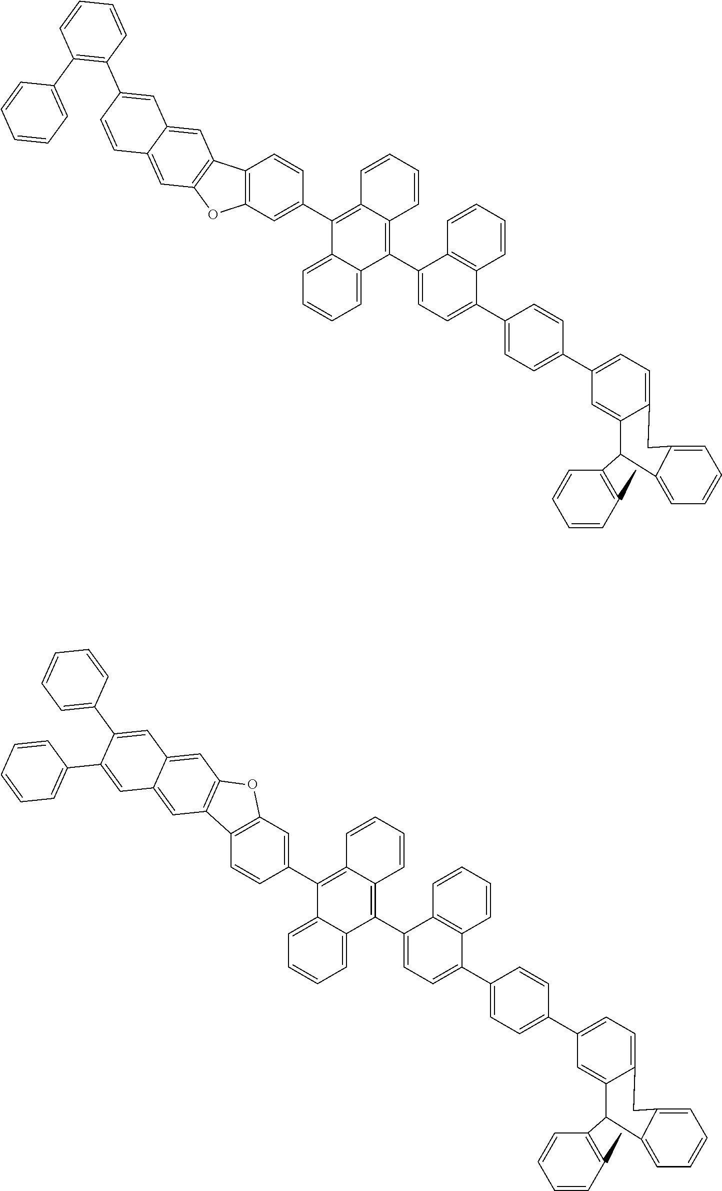

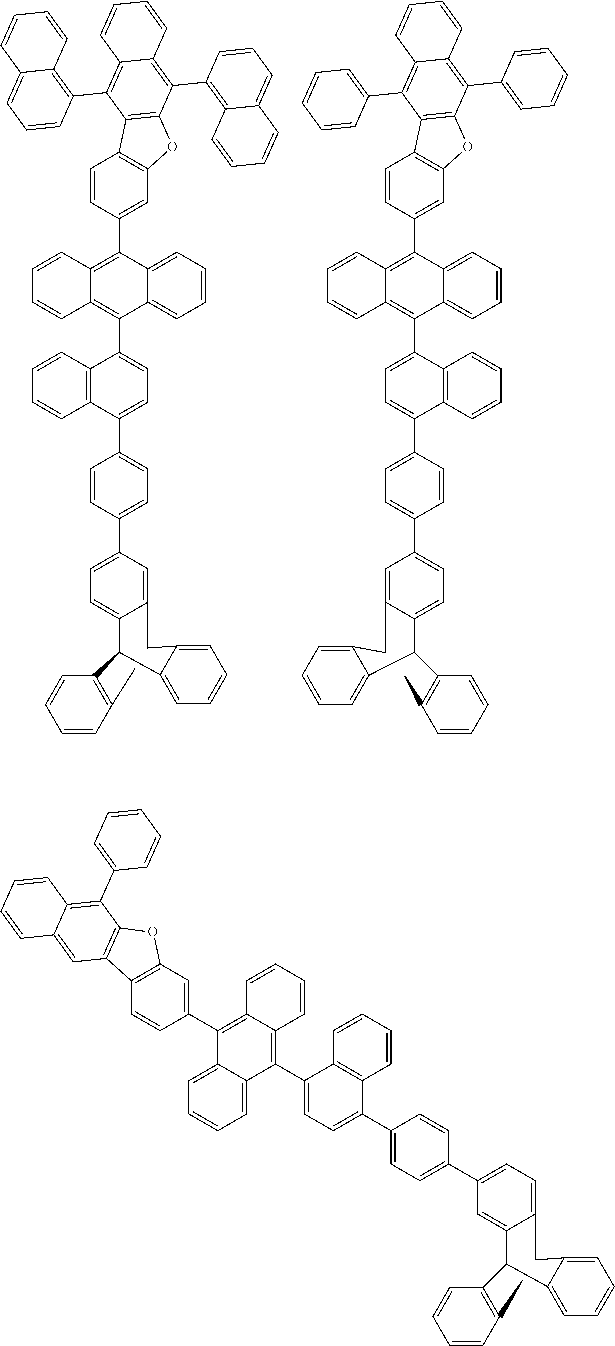

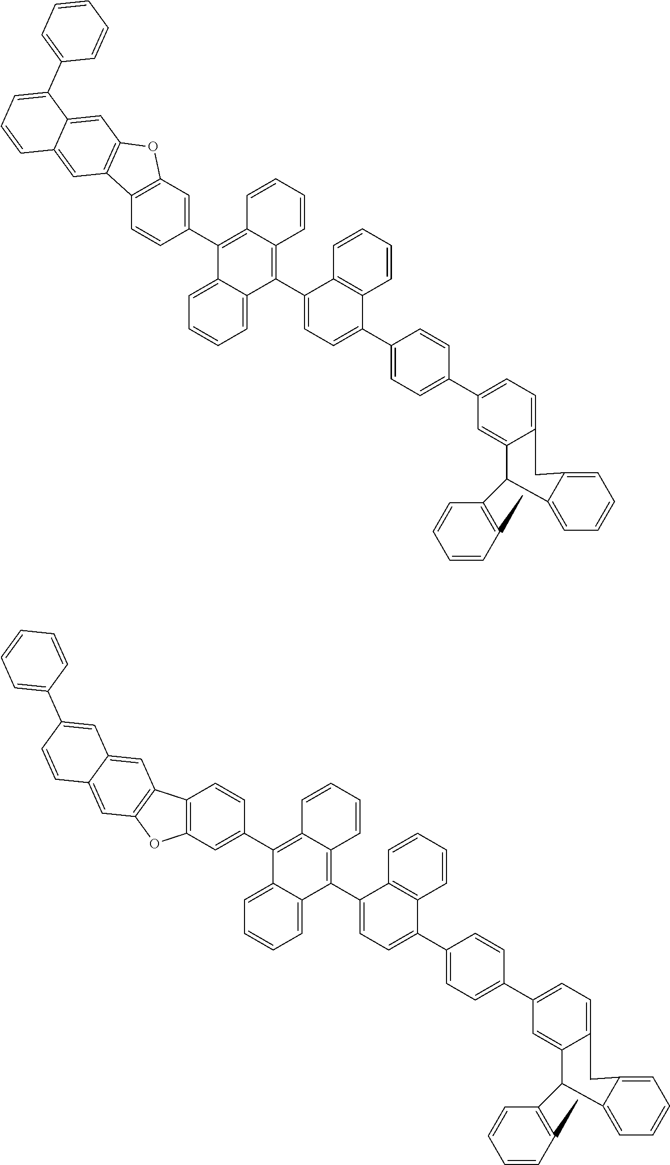

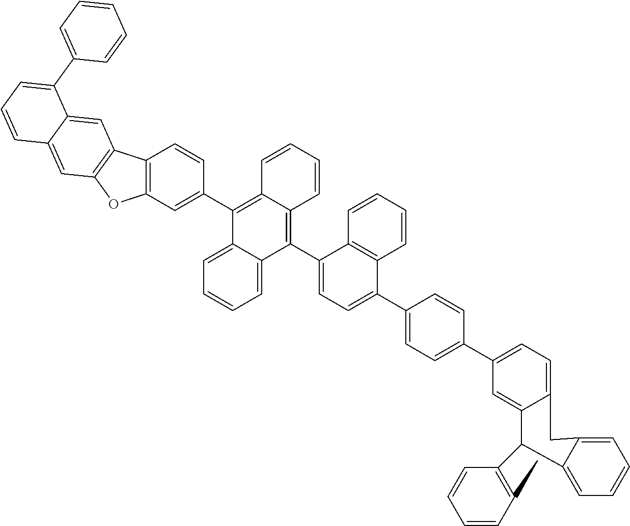

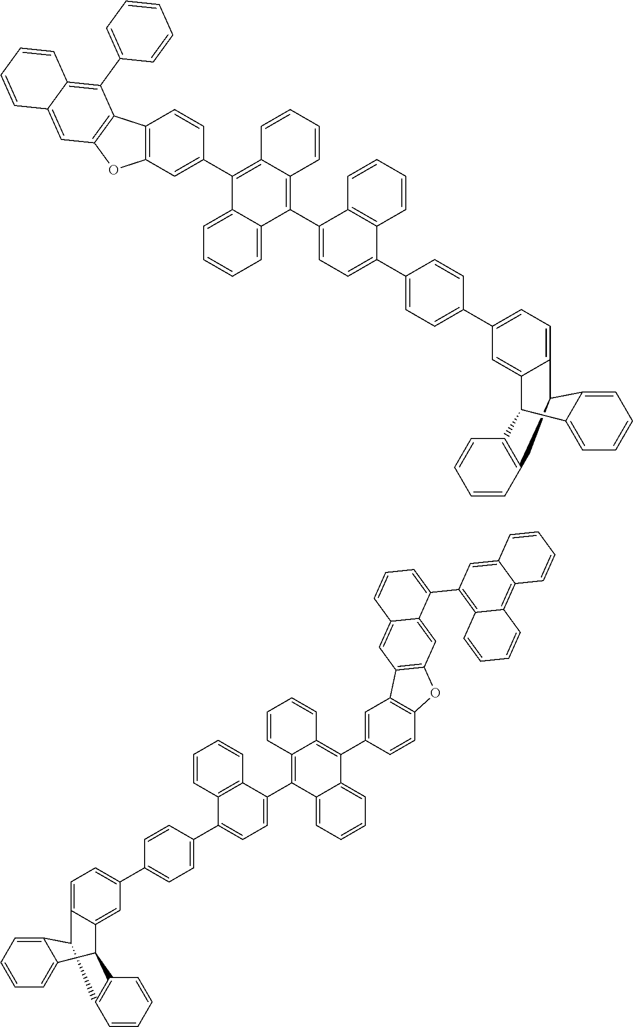

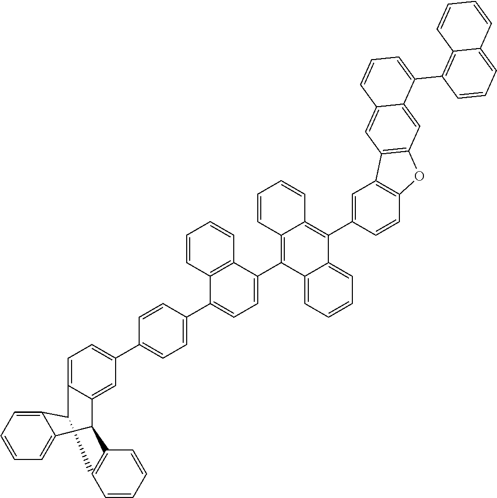 [SMILES: c1ccc2c(-c3cccc4cc5c(cc34)oc3ccc(-c4c6ccccc6c(-c6ccc(-c7ccc(-c8ccc9c(c8)[C@H]8c%10ccccc%10[C@@H]9c9ccccc98)cc7)c7ccccc67)c6ccccc46)cc35)cccc2c1]

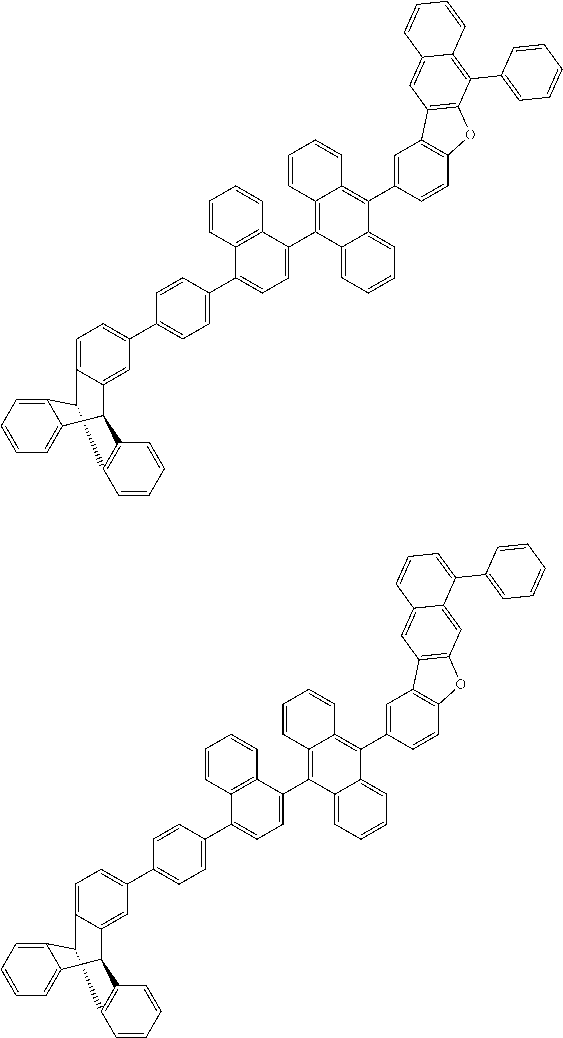 [SMILES: c1ccc(-c2c3ccccc3cc3c2oc2ccc(-c4c5ccccc5c(-c5ccc(-c6ccc(-c7ccc8c(c7)[C@H]7c9ccccc9[C@@H]8c8ccccc87)cc6)c6ccccc56)c5ccccc45)cc23)cc1 ; c1ccc(-c2cccc3cc4c(cc23)oc2ccc(-c3c5ccccc5c(-c5ccc(-c6ccc(-c7ccc8c(c7)[C@H]7c9ccccc9[C@@H]8c8ccccc87)cc6)c6ccccc56)c5ccccc35)cc24)cc1]

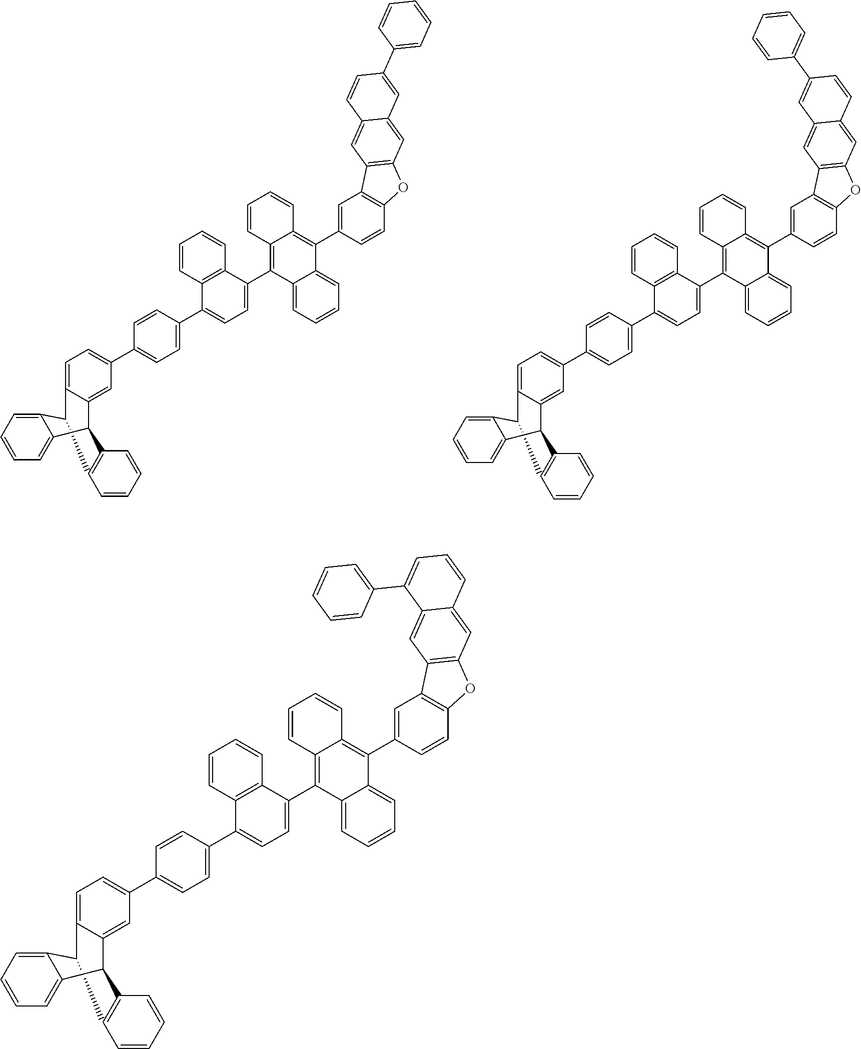 [SMILES: c1ccc(-c2ccc3cc4c(cc3c2)oc2ccc(-c3c5ccccc5c(-c5ccc(-c6ccc(-c7ccc8c(c7)[C@H]7c9ccccc9[C@@H]8c8ccccc87)cc6)c6ccccc56)c5ccccc35)cc24)cc1 ; c1ccc(-c2ccc3cc4oc5ccc(-c6c7ccccc7c(-c7ccc(-c8ccc(-c9ccc%10c(c9)[C@H]9c%11ccccc%11[C@@H]%10c%10ccccc%109)cc8)c8ccccc78)c7ccccc67)cc5c4cc3c2)cc1 ; c1ccc(-c2cccc3cc4oc5ccc(-c6c7ccccc7c(-c7ccc(-c8ccc(-c9ccc%10c(c9)[C@H]9c%11ccccc%11[C@@H]%10c%10ccccc%109)cc8)c8ccccc78)c7ccccc67)cc5c4cc23)cc1]

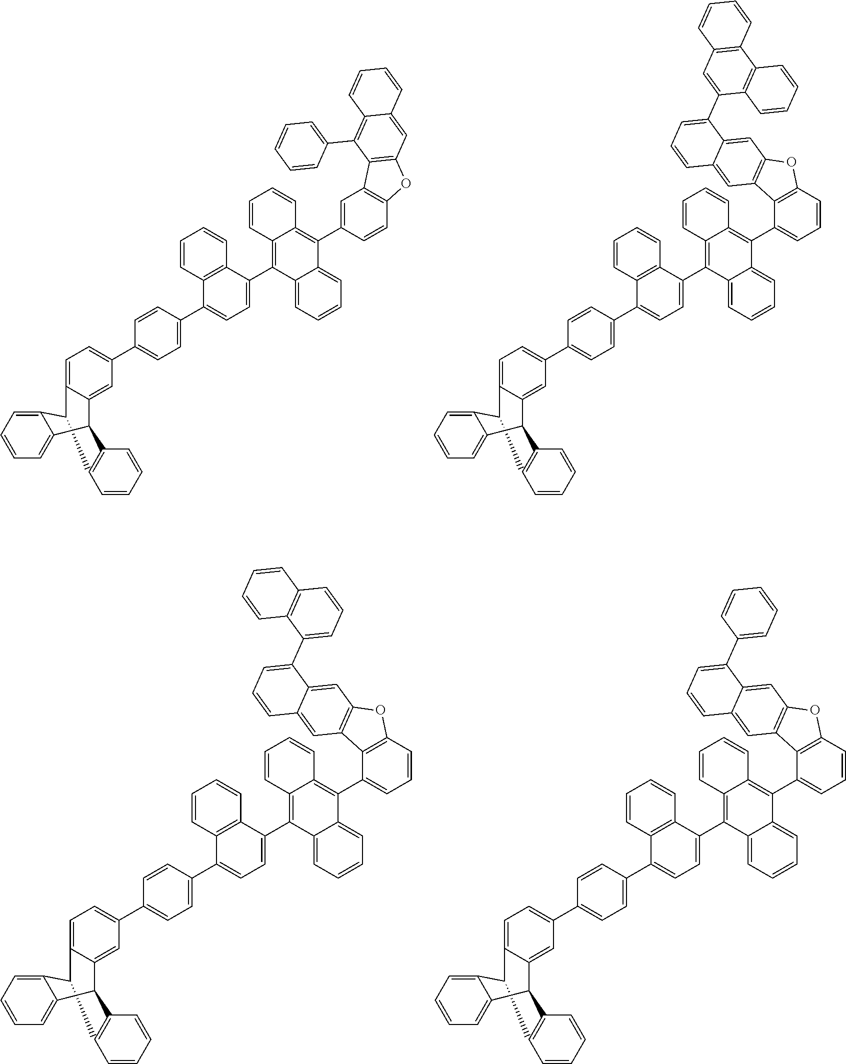

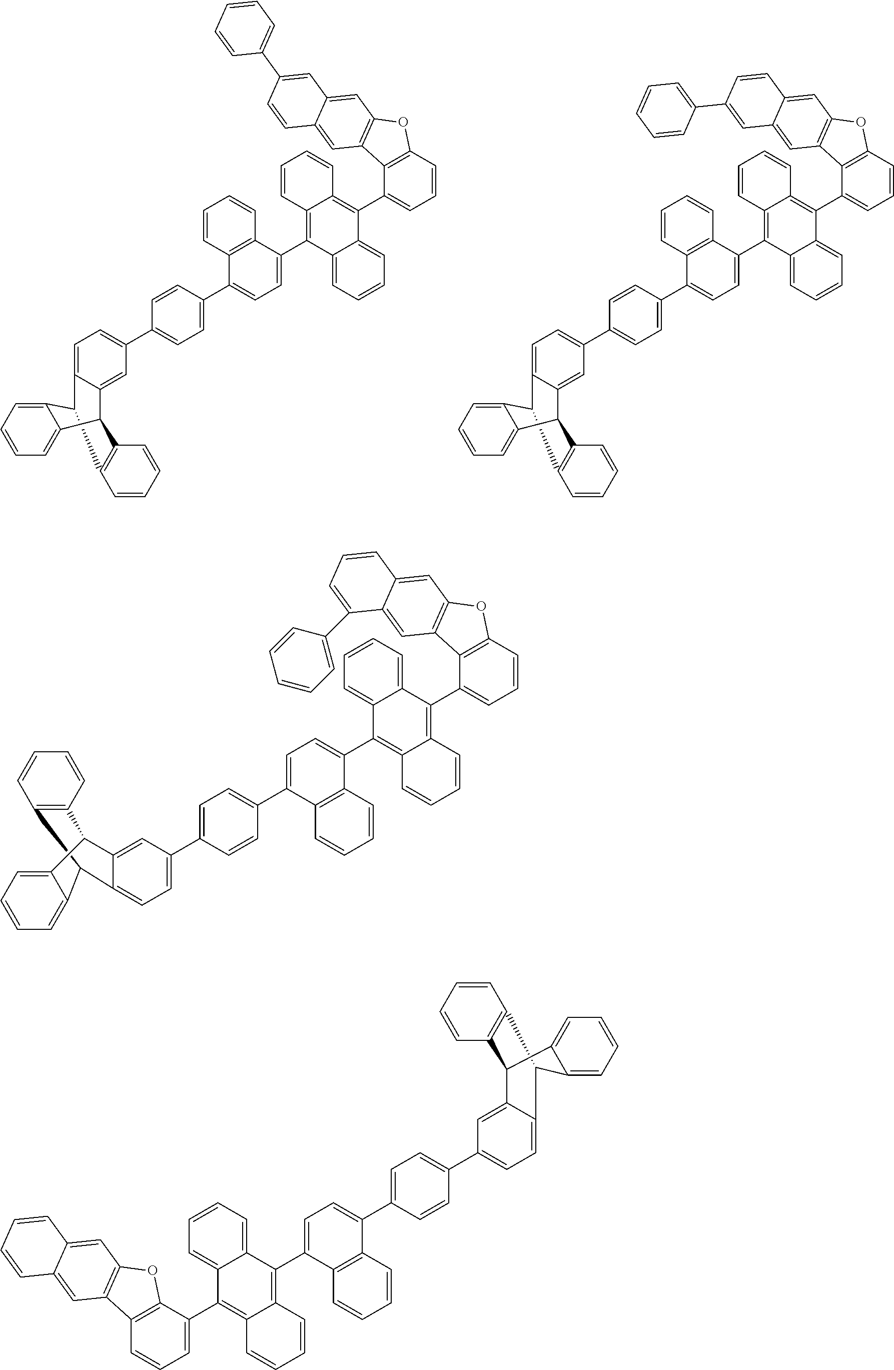

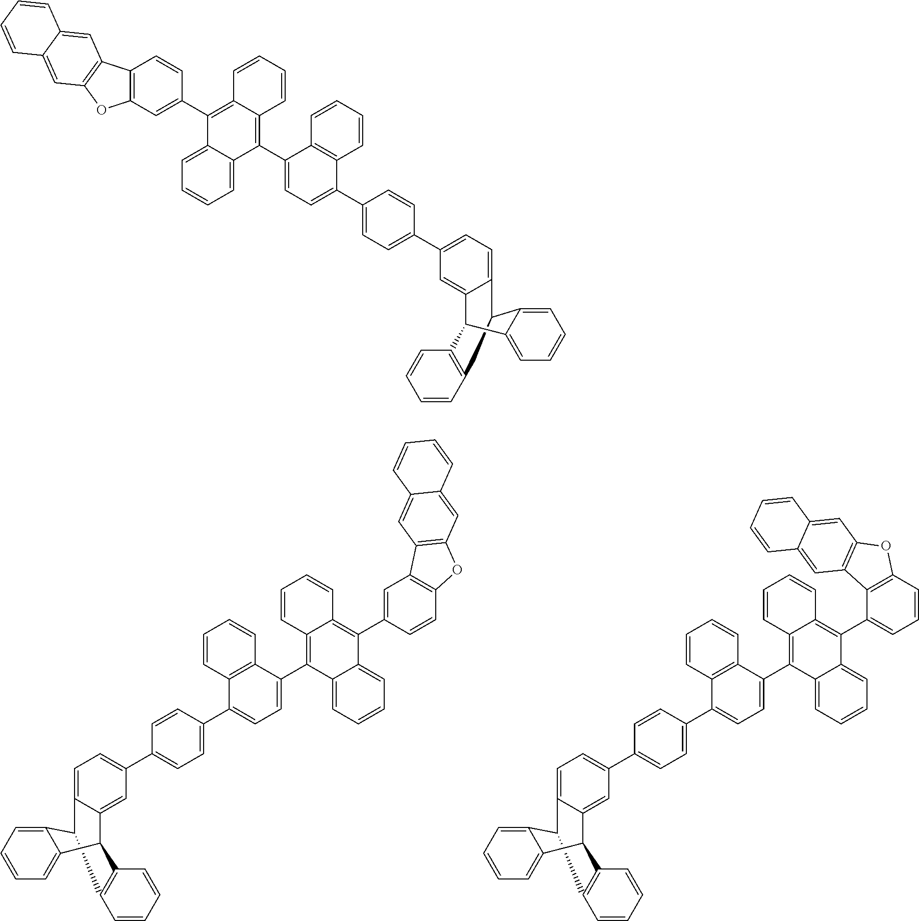

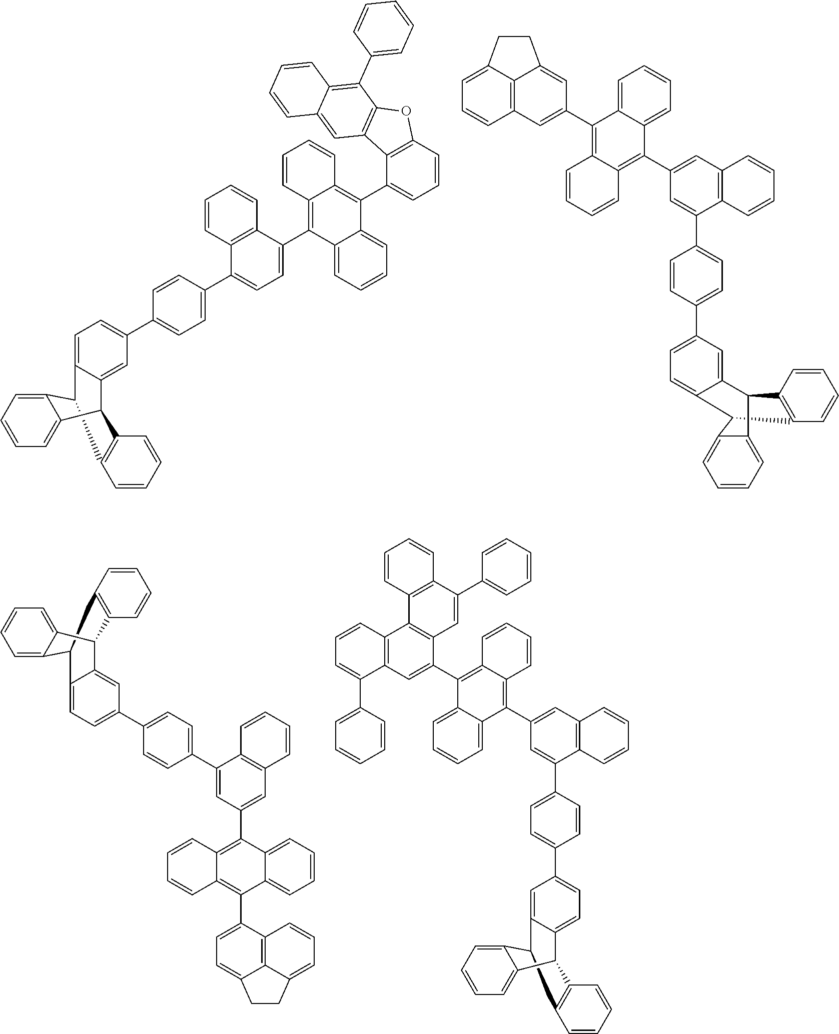

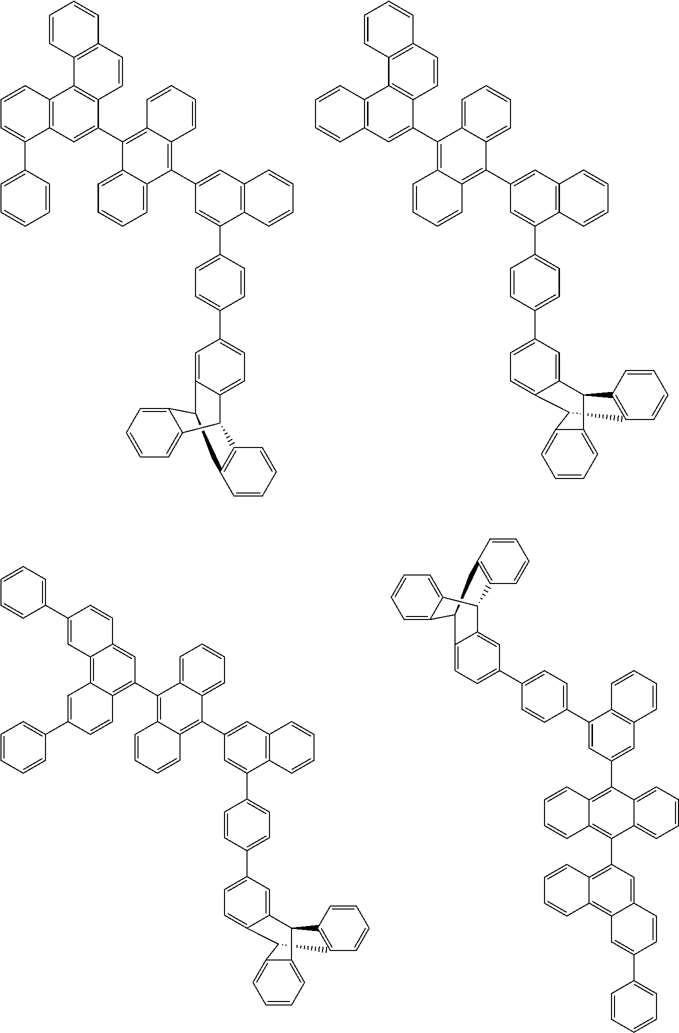

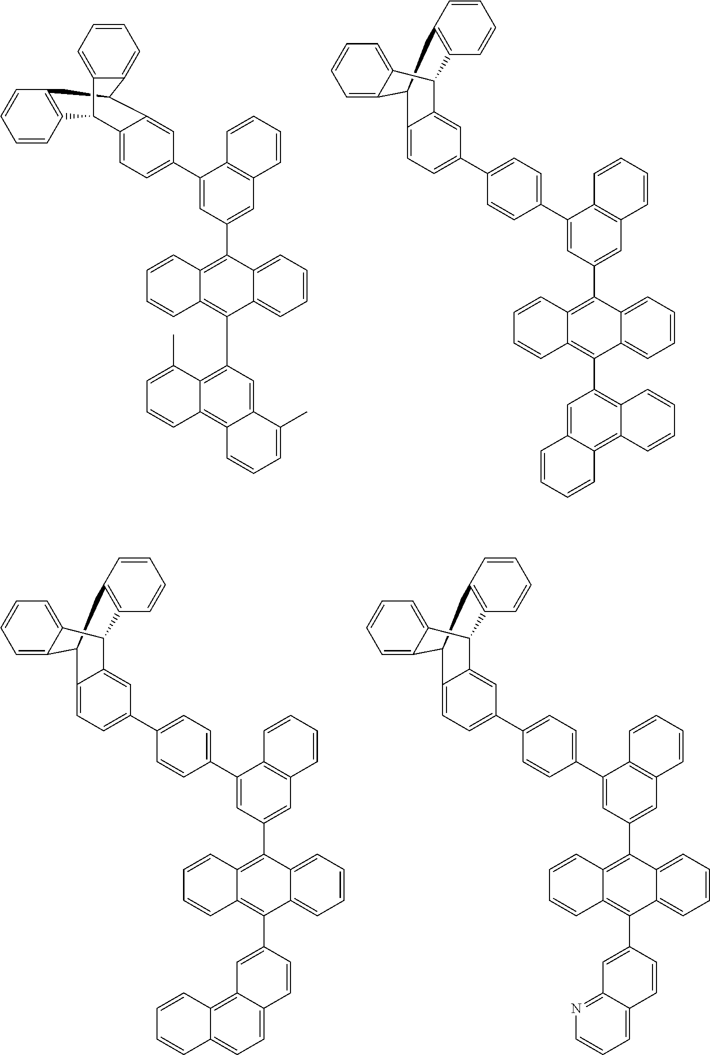

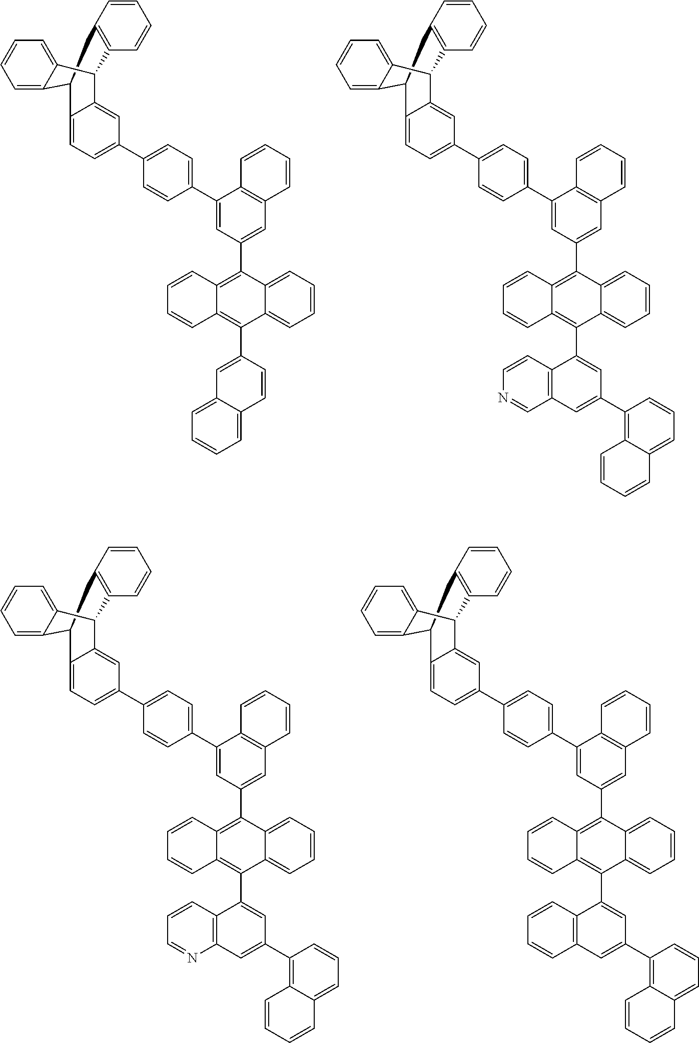

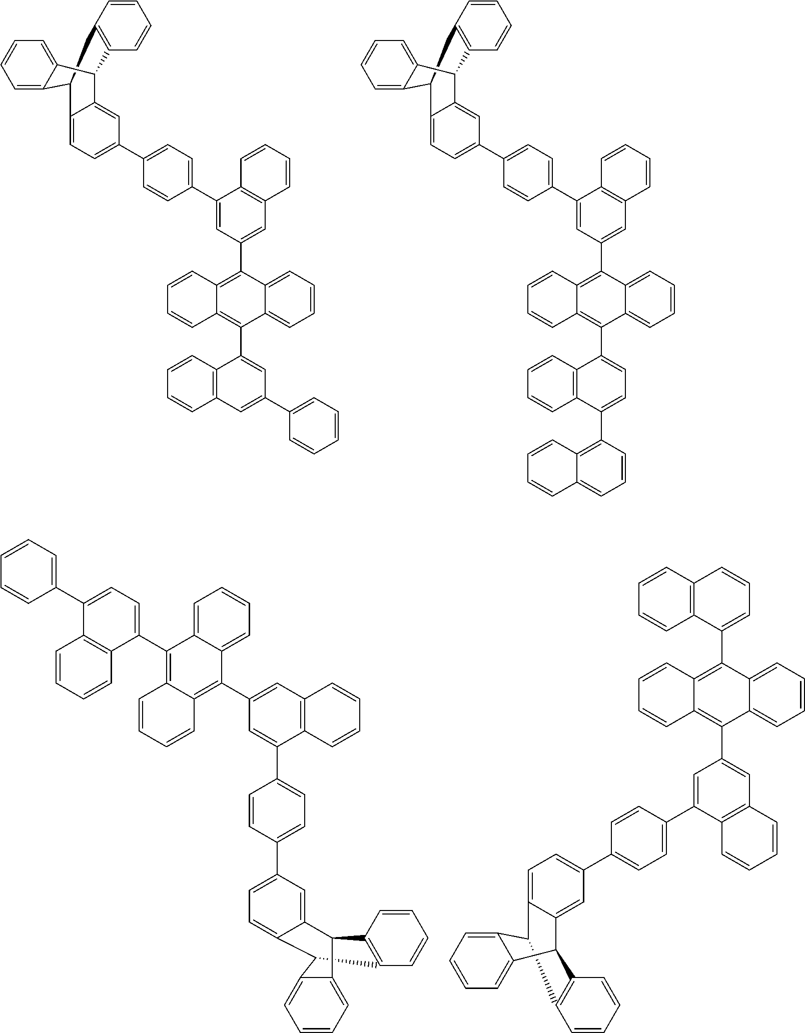

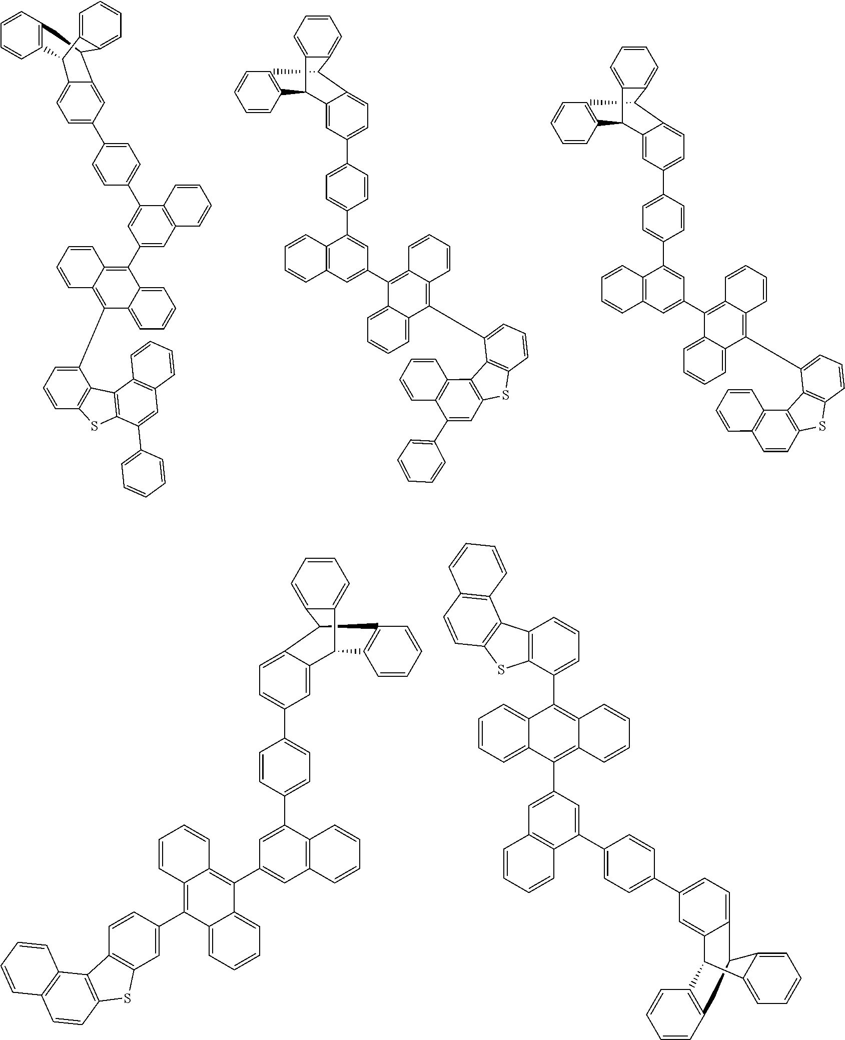

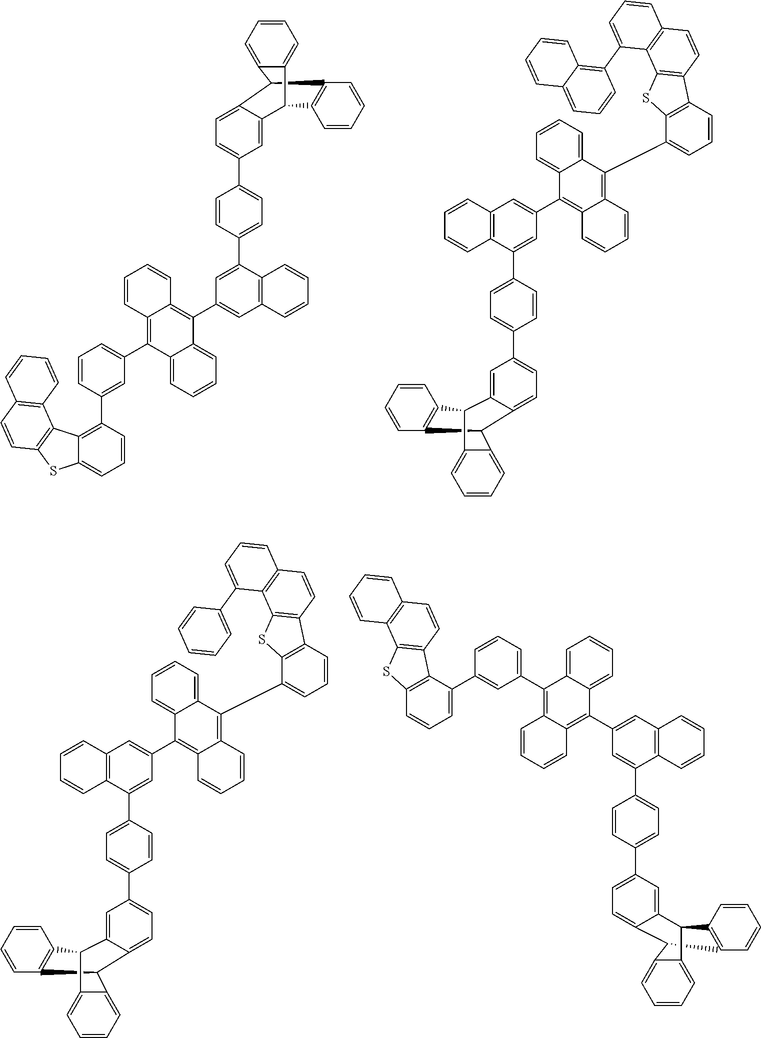

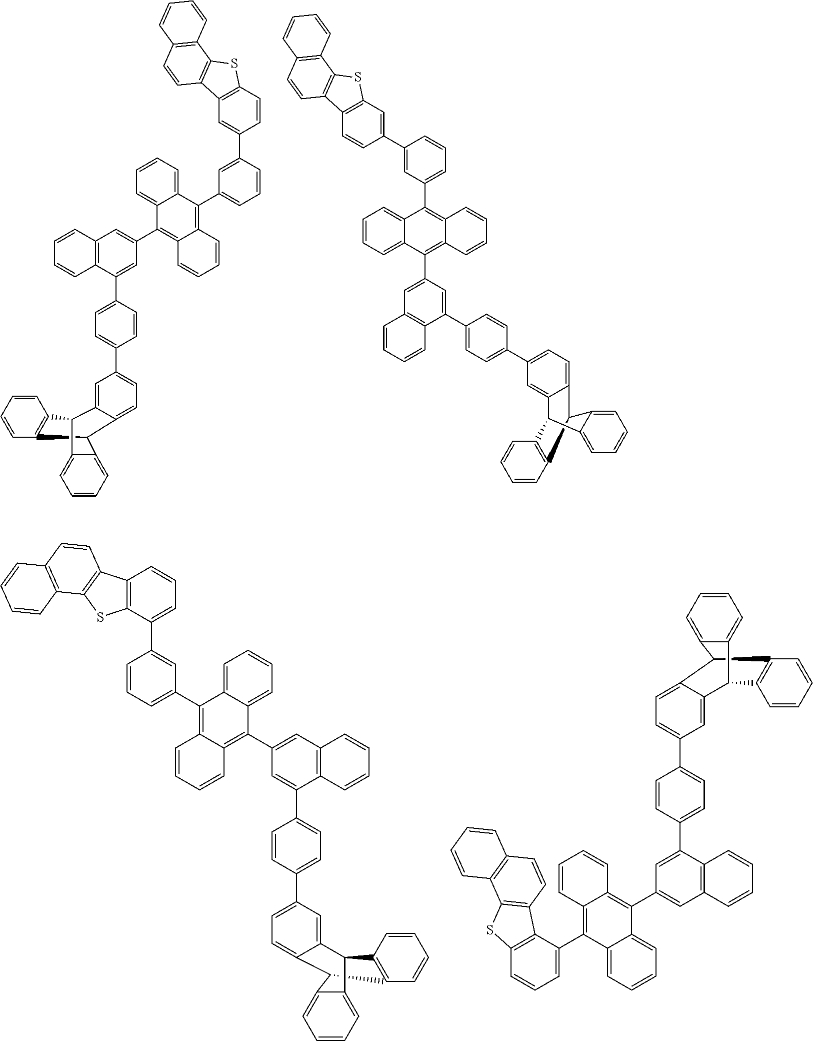

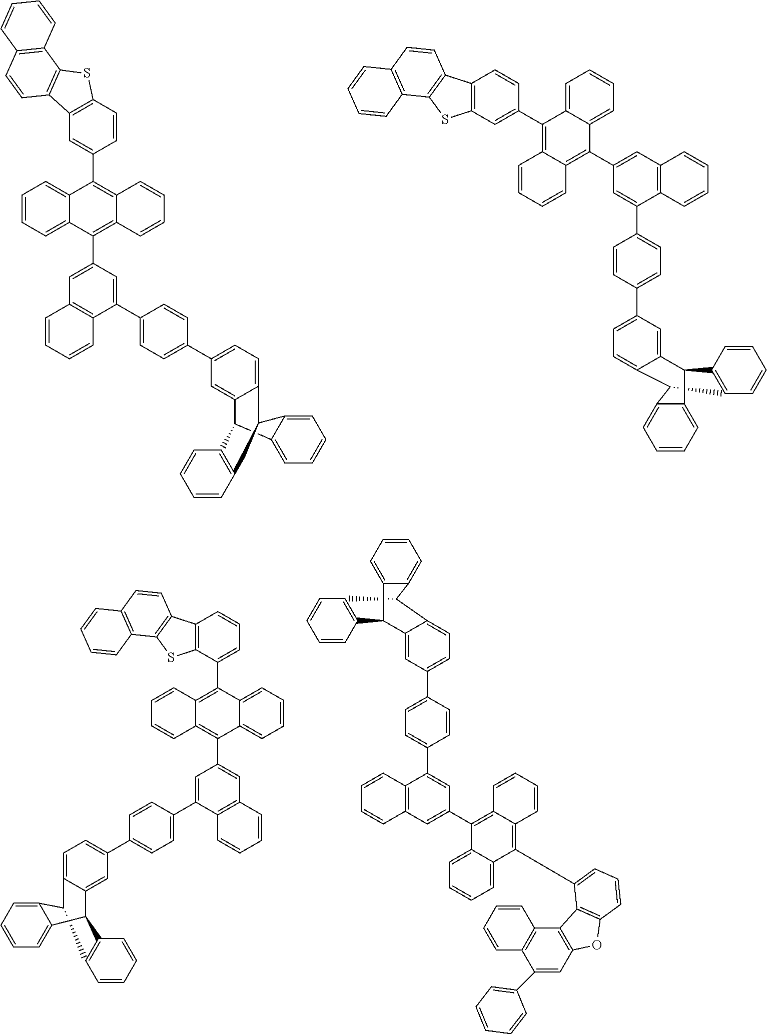

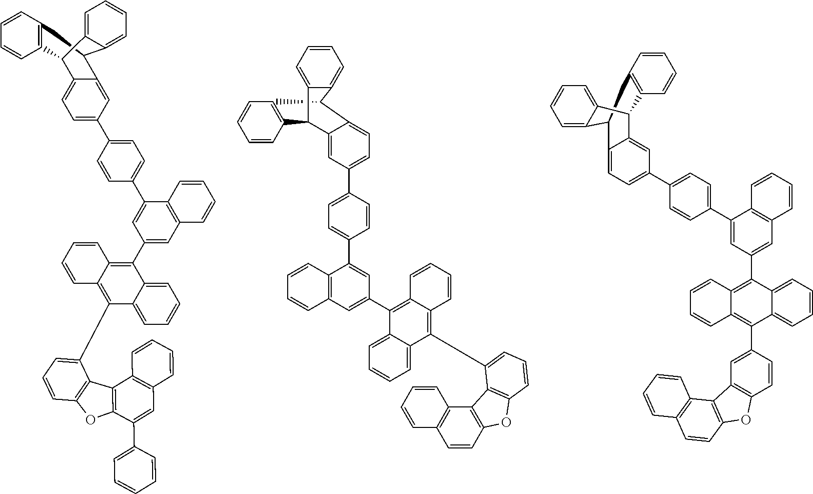 [SMILES: c1ccc(-c2cc3ccccc3c3c2oc2cccc(-c4c5ccccc5c(-c5cc(-c6ccc(-c7ccc8c(c7)[C@H]7c9ccccc9[C@@H]8c8ccccc87)cc6)c6ccccc6c5)c5ccccc45)c23)cc1 ; c1ccc2c(-c3ccc(-c4ccc5c(c4)[C@H]4c6ccccc6[C@@H]5c5ccccc54)cc3)cc(-c3c4ccccc4c(-c4cccc5oc6ccc7ccccc7c6c45)c4ccccc34)cc2c1 ; c1ccc2c(-c3ccc(-c4ccc5c(c4)[C@H]4c6ccccc6[C@@H]5c5ccccc54)cc3)cc(-c3c4ccccc4c(-c4ccc5oc6ccc7ccccc7c6c5c4)c4ccccc34)cc2c1]

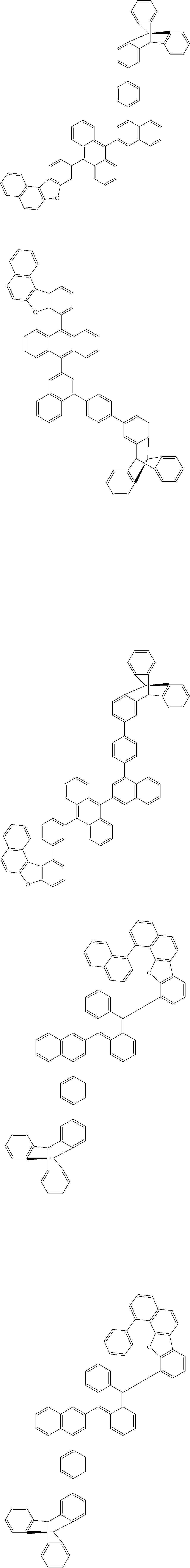

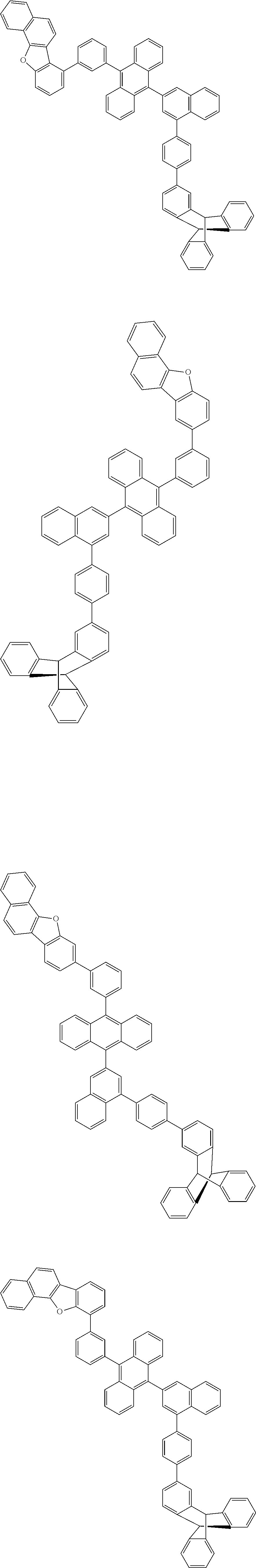

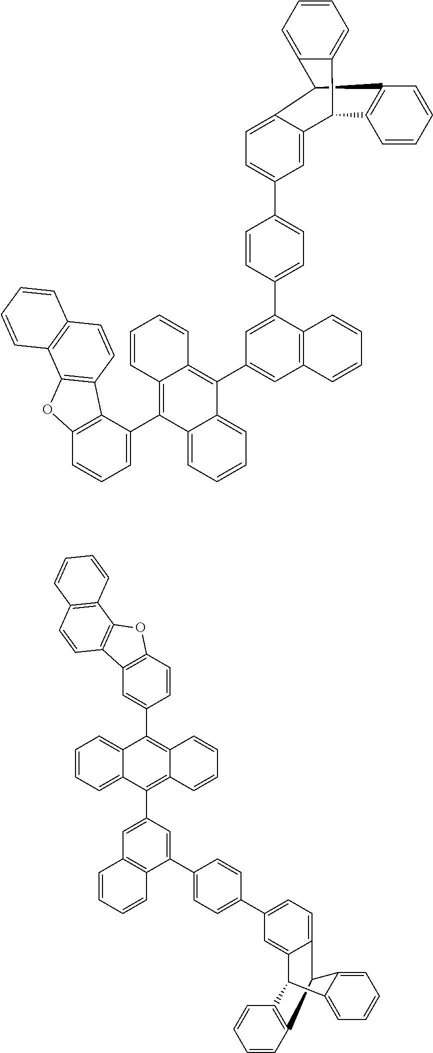 [SMILES: c1ccc2c(-c3ccc(-c4ccc5c(c4)[C@H]4c6ccccc6[C@@H]5c5ccccc54)cc3)cc(-c3c4ccccc4c(-c4cccc5oc6c7ccccc7ccc6c45)c4ccccc34)cc2c1 ; c1ccc2c(-c3ccc(-c4ccc5c(c4)[C@H]4c6ccccc6[C@@H]5c5ccccc54)cc3)cc(-c3c4ccccc4c(-c4ccc5oc6c7ccccc7ccc6c5c4)c4ccccc34)cc2c1]

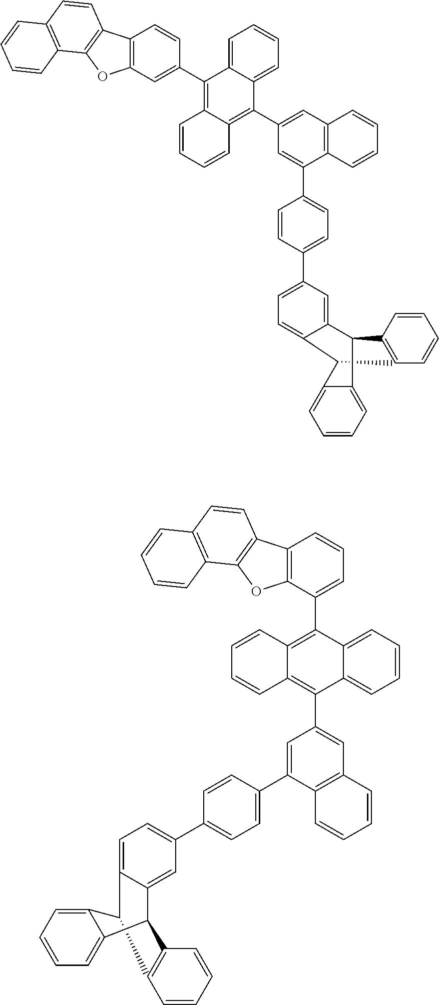 [SMILES: c1ccc2c(-c3ccc(-c4ccc5c(c4)[C@H]4c6ccccc6[C@@H]5c5ccccc54)cc3)cc(-c3c4ccccc4c(-c4ccc5c(c4)oc4c6ccccc6ccc54)c4ccccc34)cc2c1 ; c1ccc2c(-c3ccc(-c4ccc5c(c4)[C@H]4c6ccccc6[C@@H]5c5ccccc54)cc3)cc(-c3c4ccccc4c(-c4cccc5c4oc4c6ccccc6ccc54)c4ccccc34)cc2c1]

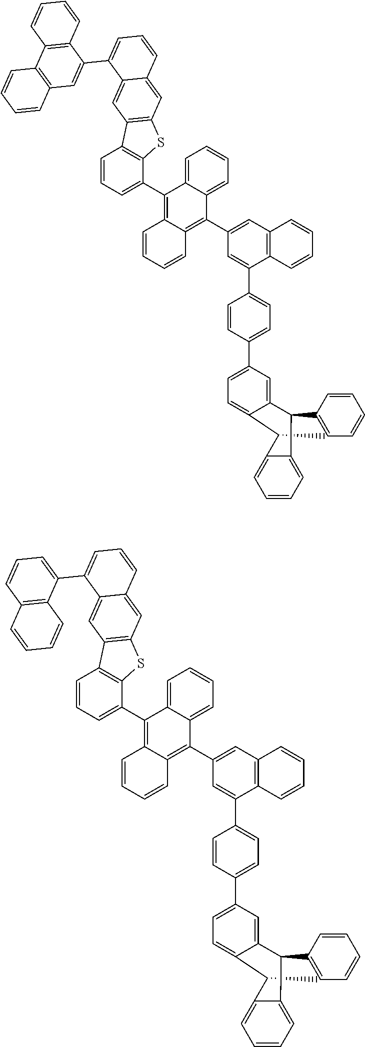 [SMILES: c1ccc2c(-c3ccc(-c4ccc5c(c4)[C@H]4c6ccccc6[C@@H]5c5ccccc54)cc3)cc(-c3c4ccccc4c(-c4cccc5c4sc4cc6cccc(-c7cc8ccccc8c8ccccc78)c6cc45)c4ccccc34)cc2c1 ; c1ccc2c(-c3ccc(-c4ccc5c(c4)[C@H]4c6ccccc6[C@@H]5c5ccccc54)cc3)cc(-c3c4ccccc4c(-c4cccc5c4sc4cc6cccc(-c7cccc8ccccc78)c6cc45)c4ccccc34)cc2c1]

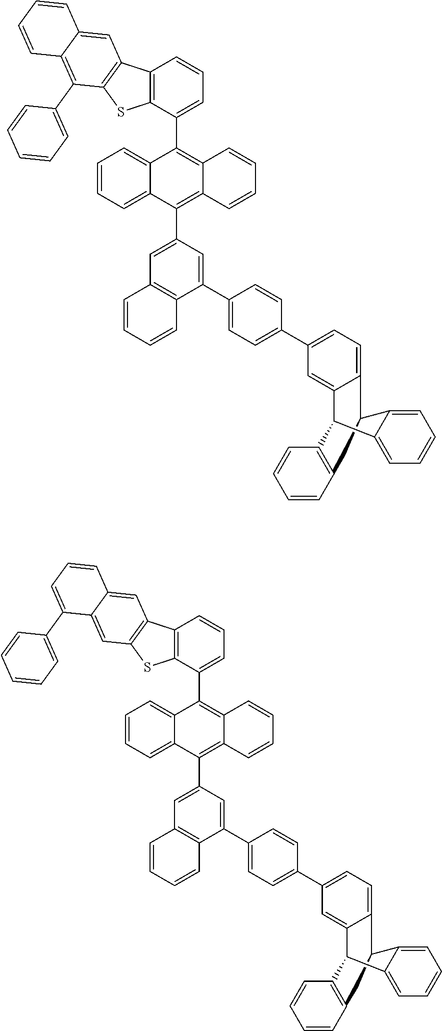 [SMILES: c1ccc(-c2c3ccccc3cc3c2sc2c(-c4c5ccccc5c(-c5cc(-c6ccc(-c7ccc8c(c7)[C@H]7c9ccccc9[C@@H]8c8ccccc87)cc6)c6ccccc6c5)c5ccccc45)cccc23)cc1 ; c1ccc(-c2cccc3cc4c(cc23)sc2c(-c3c5ccccc5c(-c5cc(-c6ccc(-c7ccc8c(c7)[C@H]7c9ccccc9[C@@H]8c8ccccc87)cc6)c6ccccc6c5)c5ccccc35)cccc24)cc1]

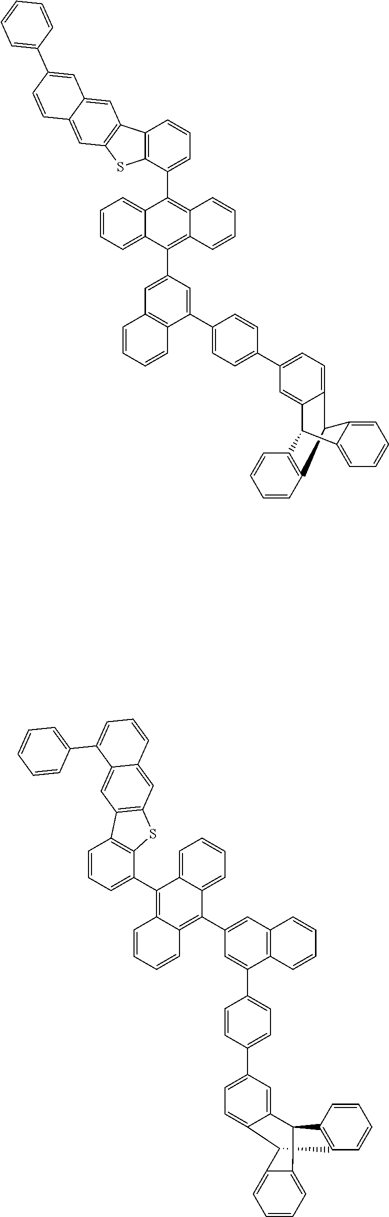 [SMILES: c1ccc(-c2ccc3cc4sc5c(-c6c7ccccc7c(-c7cc(-c8ccc(-c9ccc%10c(c9)[C@H]9c%11ccccc%11[C@@H]%10c%10ccccc%109)cc8)c8ccccc8c7)c7ccccc67)cccc5c4cc3c2)cc1 ; c1ccc(-c2cccc3cc4sc5c(-c6c7ccccc7c(-c7cc(-c8ccc(-c9ccc%10c(c9)[C@H]9c%11ccccc%11[C@@H]%10c%10ccccc%109)cc8)c8ccccc8c7)c7ccccc67)cccc5c4cc23)cc1]

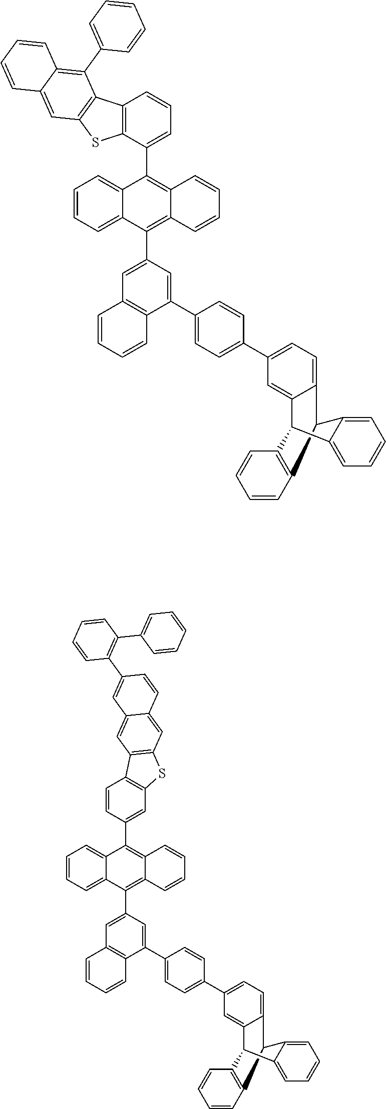 [SMILES: c1ccc(-c2c3ccccc3cc3sc4c(-c5c6ccccc6c(-c6cc(-c7ccc(-c8ccc9c(c8)[C@H]8c%10ccccc%10[C@@H]9c9ccccc98)cc7)c7ccccc7c6)c6ccccc56)cccc4c23)cc1 ; c1ccc(-c2ccccc2-c2ccc3cc4sc5cc(-c6c7ccccc7c(-c7cc(-c8ccc(-c9ccc%10c(c9)[C@H]9c%11ccccc%11[C@@H]%10c%10ccccc%109)cc8)c8ccccc8c7)c7ccccc67)ccc5c4cc3c2)cc1]

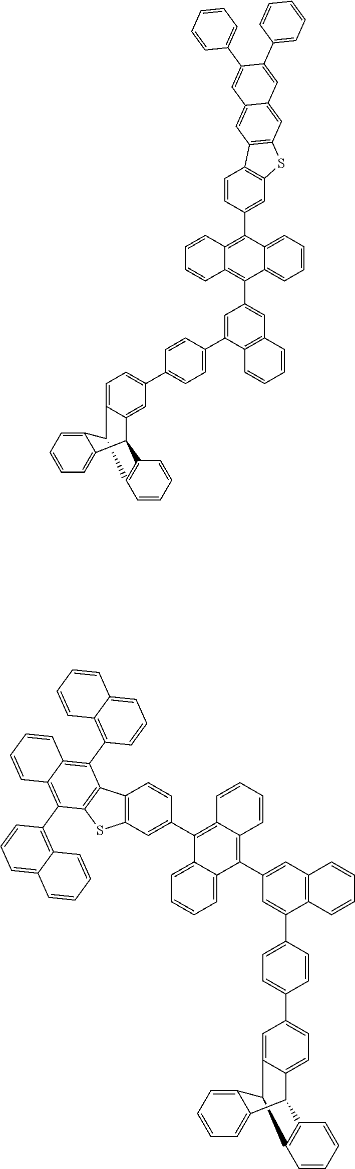 [SMILES: c1ccc(-c2cc3cc4sc5cc(-c6c7ccccc7c(-c7cc(-c8ccc(-c9ccc%10c(c9)[C@H]9c%11ccccc%11[C@@H]%10c%10ccccc%109)cc8)c8ccccc8c7)c7ccccc67)ccc5c4cc3cc2-c2ccccc2)cc1 ; c1ccc2c(-c3ccc(-c4ccc5c(c4)[C@H]4c6ccccc6[C@@H]5c5ccccc54)cc3)cc(-c3c4ccccc4c(-c4ccc5c(c4)sc4c(-c6cccc7ccccc67)c6ccccc6c(-c6cccc7ccccc67)c45)c4ccccc34)cc2c1]

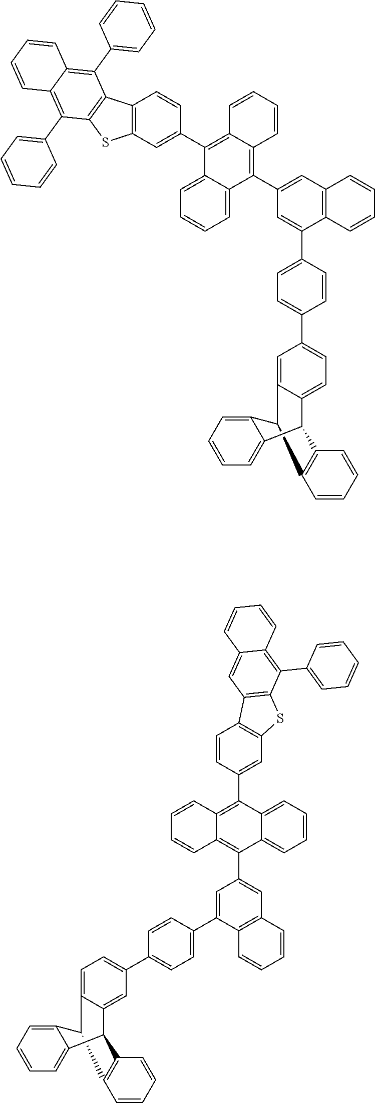 [SMILES: c1ccc(-c2c3ccccc3c(-c3ccccc3)c3c2sc2cc(-c4c5ccccc5c(-c5cc(-c6ccc(-c7ccc8c(c7)[C@H]7c9ccccc9[C@@H]8c8ccccc87)cc6)c6ccccc6c5)c5ccccc45)ccc23)cc1 ; c1ccc(-c2c3ccccc3cc3c2sc2cc(-c4c5ccccc5c(-c5cc(-c6ccc(-c7ccc8c(c7)[C@H]7c9ccccc9[C@@H]8c8ccccc87)cc6)c6ccccc6c5)c5ccccc45)ccc23)cc1]

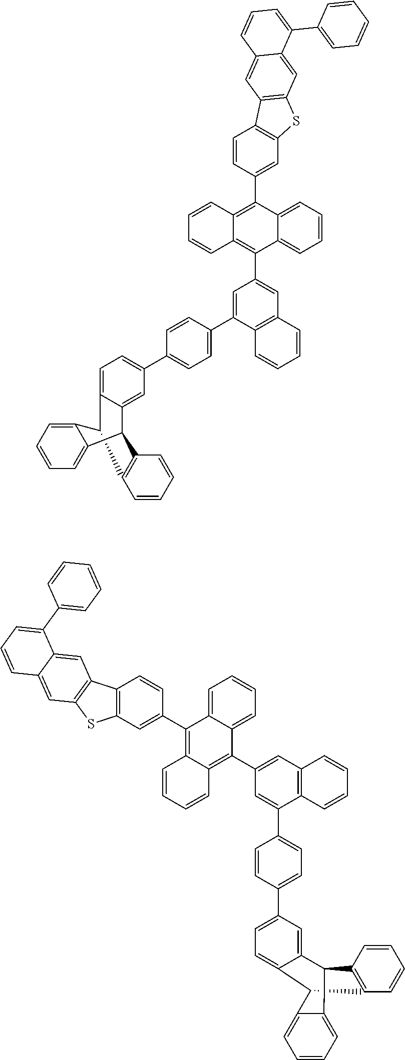 [SMILES: c1ccc(-c2cccc3cc4c(cc23)sc2cc(-c3c5ccccc5c(-c5cc(-c6ccc(-c7ccc8c(c7)[C@H]7c9ccccc9[C@@H]8c8ccccc87)cc6)c6ccccc6c5)c5ccccc35)ccc24)cc1 ; c1ccc(-c2cccc3cc4sc5cc(-c6c7ccccc7c(-c7cc(-c8ccc(-c9ccc%10c(c9)[C@H]9c%11ccccc%11[C@@H]%10c%10ccccc%109)cc8)c8ccccc8c7)c7ccccc67)ccc5c4cc23)cc1]

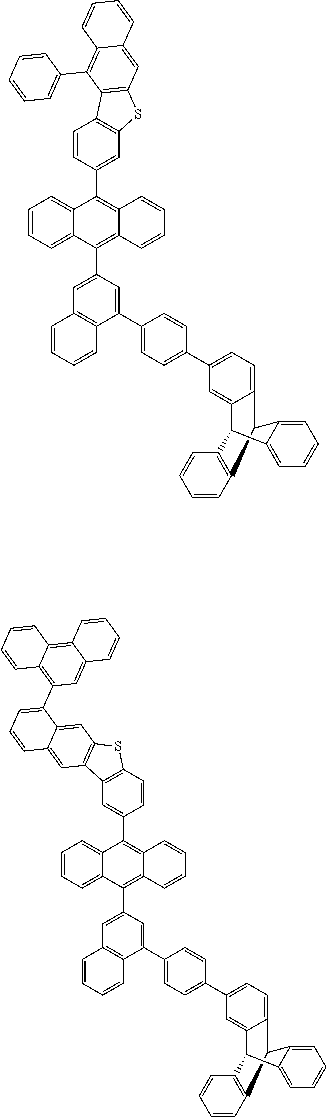 [SMILES: c1ccc(-c2c3ccccc3cc3sc4cc(-c5c6ccccc6c(-c6cc(-c7ccc(-c8ccc9c(c8)[C@H]8c%10ccccc%10[C@@H]9c9ccccc98)cc7)c7ccccc7c6)c6ccccc56)ccc4c23)cc1 ; c1ccc2c(-c3ccc(-c4ccc5c(c4)[C@H]4c6ccccc6[C@@H]5c5ccccc54)cc3)cc(-c3c4ccccc4c(-c4ccc5sc6cc7c(-c8cc9ccccc9c9ccccc89)cccc7cc6c5c4)c4ccccc34)cc2c1]

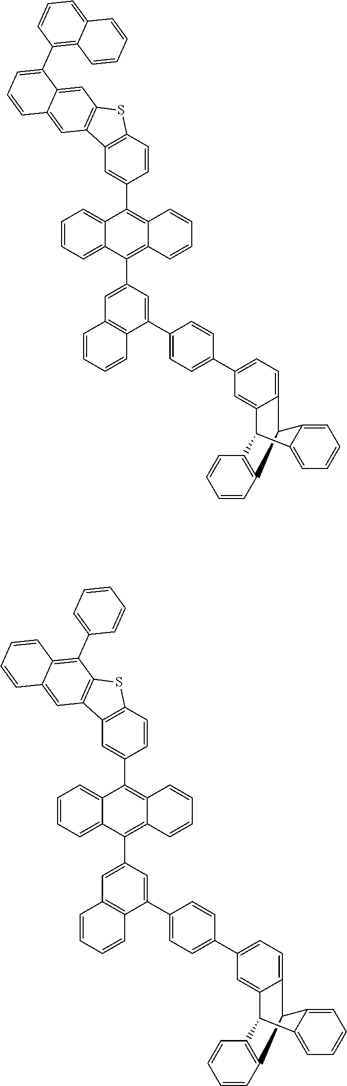 [SMILES: c1ccc2c(-c3ccc(-c4ccc5c(c4)[C@H]4c6ccccc6[C@@H]5c5ccccc54)cc3)cc(-c3c4ccccc4c(-c4ccc5sc6cc7c(-c8cccc9ccccc89)cccc7cc6c5c4)c4ccccc34)cc2c1 ; c1ccc(-c2c3ccccc3cc3c2sc2ccc(-c4c5ccccc5c(-c5cc(-c6ccc(-c7ccc8c(c7)[C@H]7c9ccccc9[C@@H]8c8ccccc87)cc6)c6ccccc6c5)c5ccccc45)cc23)cc1]

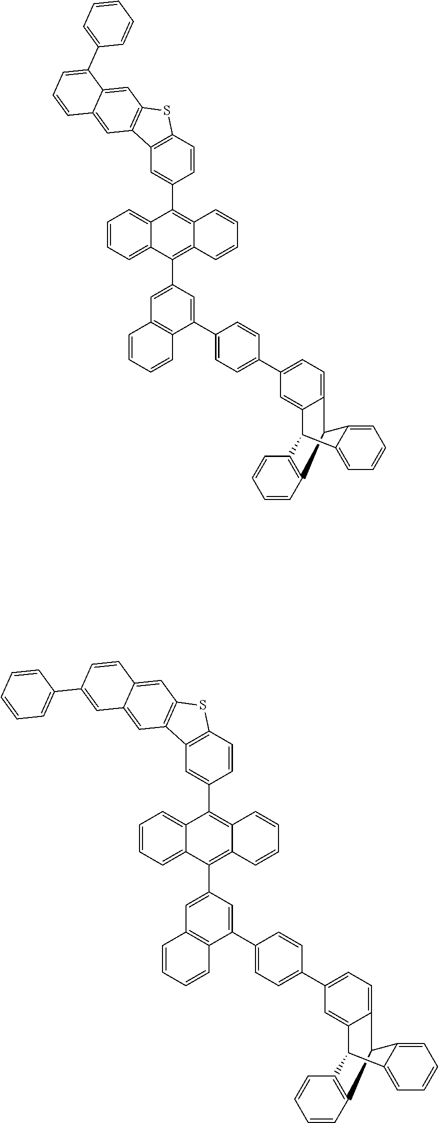 [SMILES: c1ccc(-c2cccc3cc4c(cc23)sc2ccc(-c3c5ccccc5c(-c5cc(-c6ccc(-c7ccc8c(c7)[C@H]7c9ccccc9[C@@H]8c8ccccc87)cc6)c6ccccc6c5)c5ccccc35)cc24)cc1 ; c1ccc(-c2ccc3cc4sc5ccc(-c6c7ccccc7c(-c7cc(-c8ccc(-c9ccc%10c(c9)[C@H]9c%11ccccc%11[C@@H]%10c%10ccccc%109)cc8)c8ccccc8c7)c7ccccc67)cc5c4cc3c2)cc1]

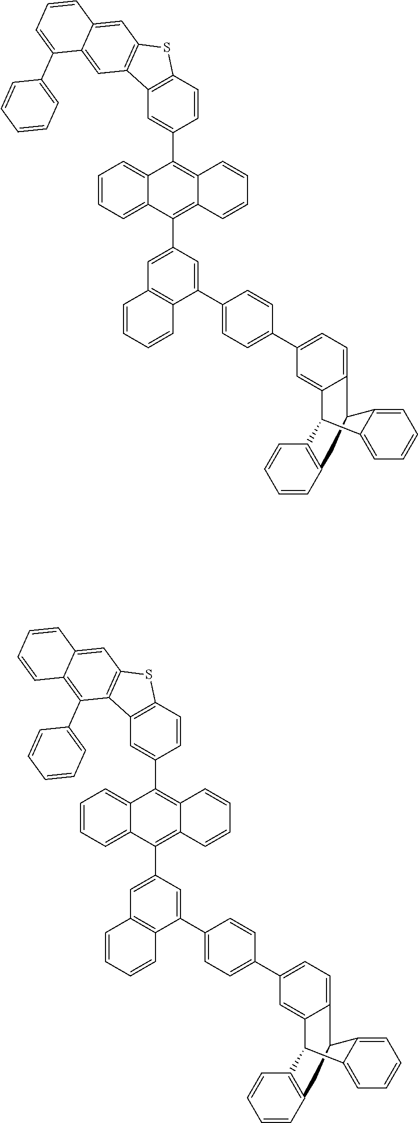 [SMILES: c1ccc(-c2cccc3cc4sc5ccc(-c6c7ccccc7c(-c7cc(-c8ccc(-c9ccc%10c(c9)[C@H]9c%11ccccc%11[C@@H]%10c%10ccccc%109)cc8)c8ccccc8c7)c7ccccc67)cc5c4cc23)cc1 ; c1ccc(-c2c3ccccc3cc3sc4ccc(-c5c6ccccc6c(-c6cc(-c7ccc(-c8ccc9c(c8)[C@H]8c%10ccccc%10[C@@H]9c9ccccc98)cc7)c7ccccc7c6)c6ccccc56)cc4c23)cc1]

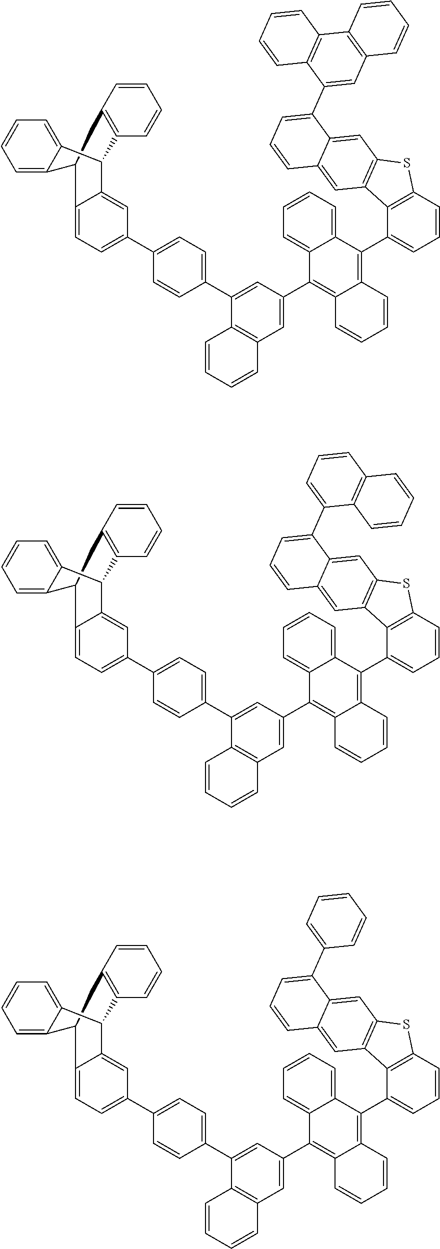 [SMILES: c1ccc2c(-c3ccc(-c4ccc5c(c4)[C@H]4c6ccccc6[C@@H]5c5ccccc54)cc3)cc(-c3c4ccccc4c(-c4cccc5sc6cc7c(-c8cc9ccccc9c9ccccc89)cccc7cc6c45)c4ccccc34)cc2c1 ; c1ccc2c(-c3ccc(-c4ccc5c(c4)[C@H]4c6ccccc6[C@@H]5c5ccccc54)cc3)cc(-c3c4ccccc4c(-c4cccc5sc6cc7c(-c8cccc9ccccc89)cccc7cc6c45)c4ccccc34)cc2c1 ; c1ccc(-c2cccc3cc4c(cc23)sc2cccc(-c3c5ccccc5c(-c5cc(-c6ccc(-c7ccc8c(c7)[C@H]7c9ccccc9[C@@H]8c8ccccc87)cc6)c6ccccc6c5)c5ccccc35)c24)cc1]

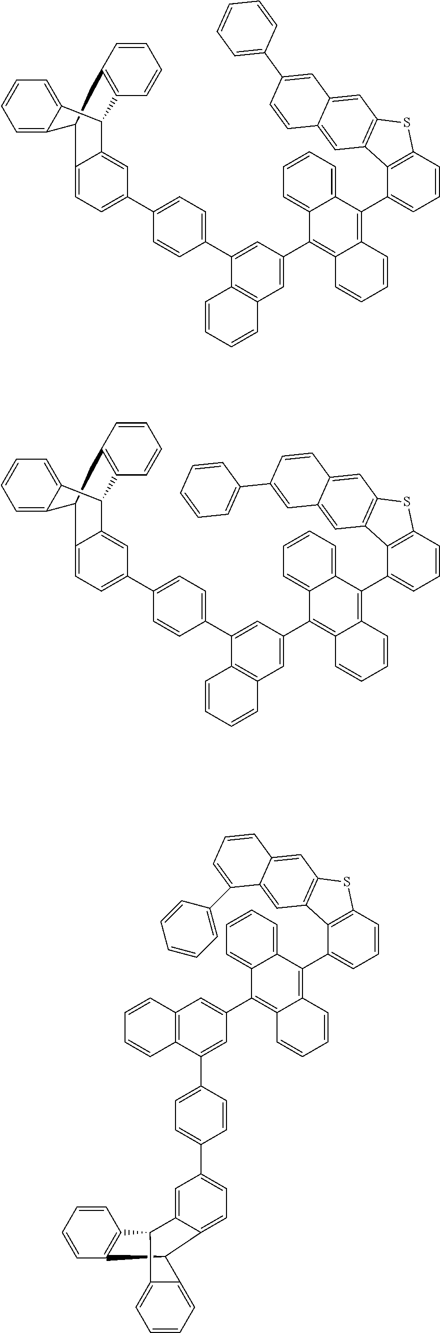 [SMILES: c1ccc(-c2ccc3cc4c(cc3c2)sc2cccc(-c3c5ccccc5c(-c5cc(-c6ccc(-c7ccc8c(c7)[C@H]7c9ccccc9[C@@H]8c8ccccc87)cc6)c6ccccc6c5)c5ccccc35)c24)cc1 ; c1ccc(-c2ccc3cc4sc5cccc(-c6c7ccccc7c(-c7cc(-c8ccc(-c9ccc%10c(c9)[C@H]9c%11ccccc%11[C@@H]%10c%10ccccc%109)cc8)c8ccccc8c7)c7ccccc67)c5c4cc3c2)cc1 ; c1ccc(-c2cccc3cc4sc5cccc(-c6c7ccccc7c(-c7cc(-c8ccc(-c9ccc%10c(c9)[C@H]9c%11ccccc%11[C@@H]%10c%10ccccc%109)cc8)c8ccccc8c7)c7ccccc67)c5c4cc23)cc1]

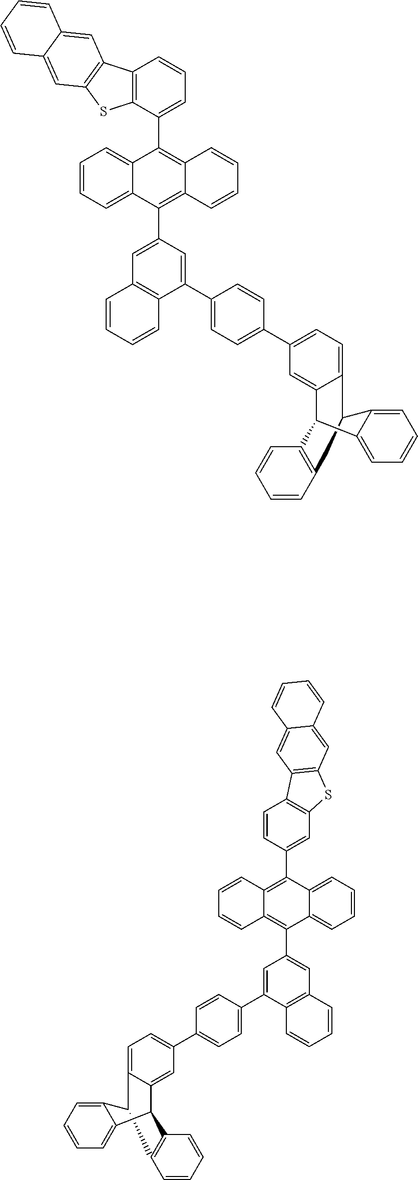 [SMILES: c1ccc2cc3c(cc2c1)sc1c(-c2c4ccccc4c(-c4cc(-c5ccc(-c6ccc7c(c6)[C@H]6c8ccccc8[C@@H]7c7ccccc76)cc5)c5ccccc5c4)c4ccccc24)cccc13 ; c1ccc2cc3c(cc2c1)sc1cc(-c2c4ccccc4c(-c4cc(-c5ccc(-c6ccc7c(c6)[C@H]6c8ccccc8[C@@H]7c7ccccc76)cc5)c5ccccc5c4)c4ccccc24)ccc13]

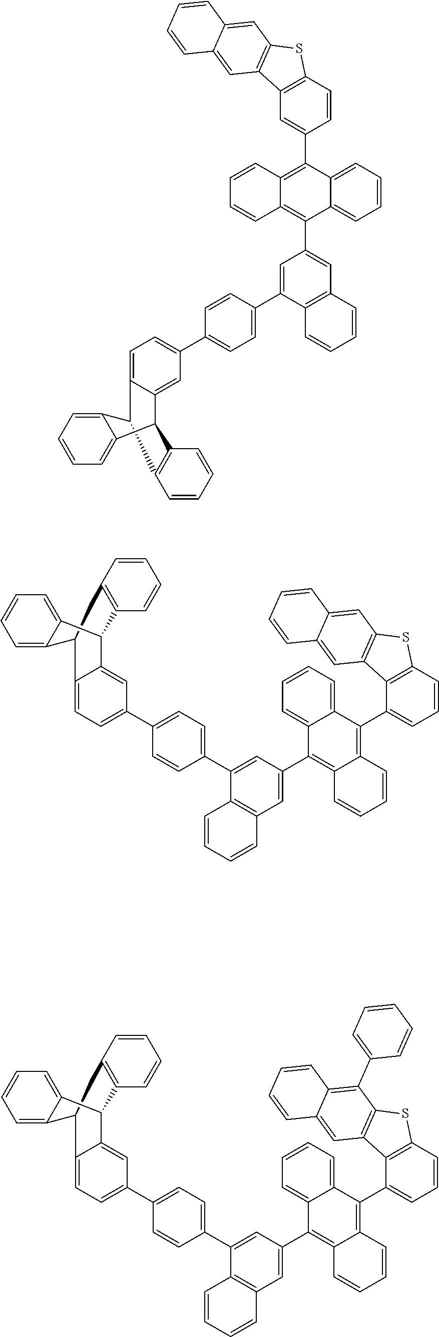 [SMILES: c1ccc2cc3c(cc2c1)sc1ccc(-c2c4ccccc4c(-c4cc(-c5ccc(-c6ccc7c(c6)[C@H]6c8ccccc8[C@@H]7c7ccccc76)cc5)c5ccccc5c4)c4ccccc24)cc13 ; c1ccc2cc3c(cc2c1)sc1cccc(-c2c4ccccc4c(-c4cc(-c5ccc(-c6ccc7c(c6)[C@H]6c8ccccc8[C@@H]7c7ccccc76)cc5)c5ccccc5c4)c4ccccc24)c13 ; c1ccc(-c2c3ccccc3cc3c2sc2cccc(-c4c5ccccc5c(-c5cc(-c6ccc(-c7ccc8c(c7)[C@H]7c9ccccc9[C@@H]8c8ccccc87)cc6)c6ccccc6c5)c5ccccc45)c23)cc1]

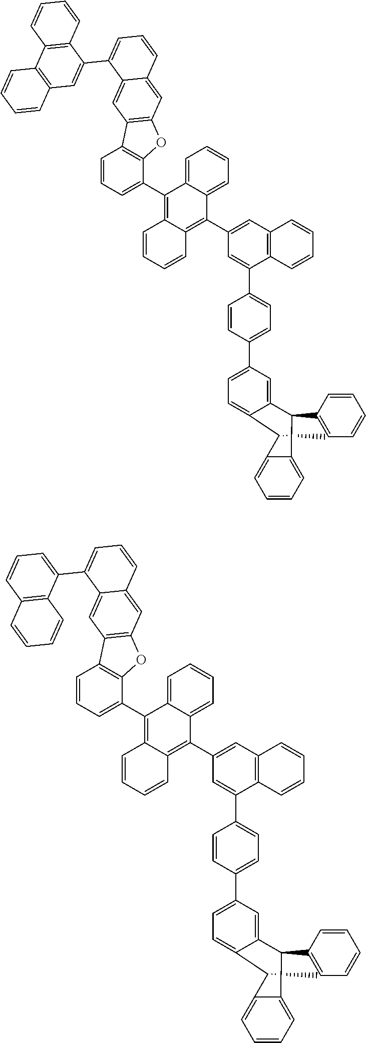 [SMILES: c1ccc2c(-c3ccc(-c4ccc5c(c4)[C@H]4c6ccccc6[C@@H]5c5ccccc54)cc3)cc(-c3c4ccccc4c(-c4cccc5c4oc4cc6cccc(-c7cc8ccccc8c8ccccc78)c6cc45)c4ccccc34)cc2c1 ; c1ccc2c(-c3ccc(-c4ccc5c(c4)[C@H]4c6ccccc6[C@@H]5c5ccccc54)cc3)cc(-c3c4ccccc4c(-c4cccc5c4oc4cc6cccc(-c7cccc8ccccc78)c6cc45)c4ccccc34)cc2c1]

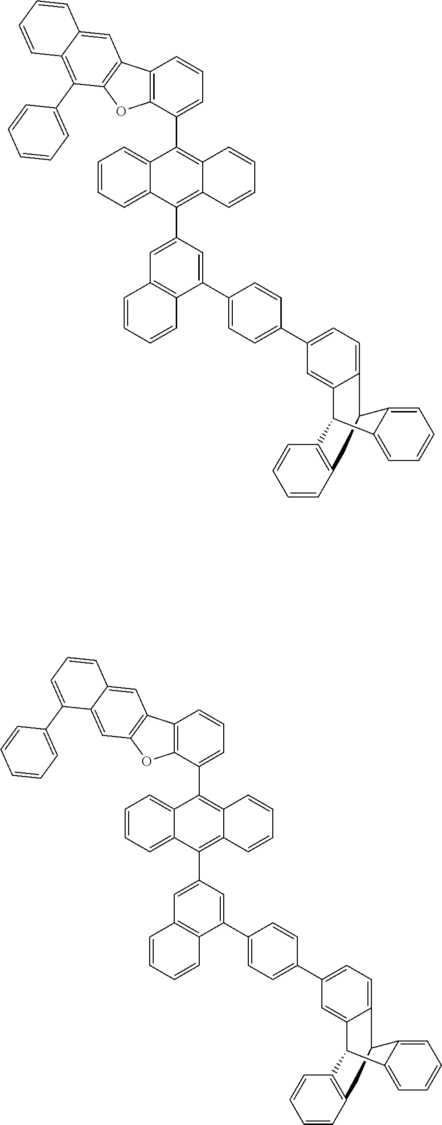 [SMILES: c1ccc(-c2c3ccccc3cc3c2oc2c(-c4c5ccccc5c(-c5cc(-c6ccc(-c7ccc8c(c7)[C@H]7c9ccccc9[C@@H]8c8ccccc87)cc6)c6ccccc6c5)c5ccccc45)cccc23)cc1 ; c1ccc(-c2cccc3cc4c(cc23)oc2c(-c3c5ccccc5c(-c5cc(-c6ccc(-c7ccc8c(c7)[C@H]7c9ccccc9[C@@H]8c8ccccc87)cc6)c6ccccc6c5)c5ccccc35)cccc24)cc1]

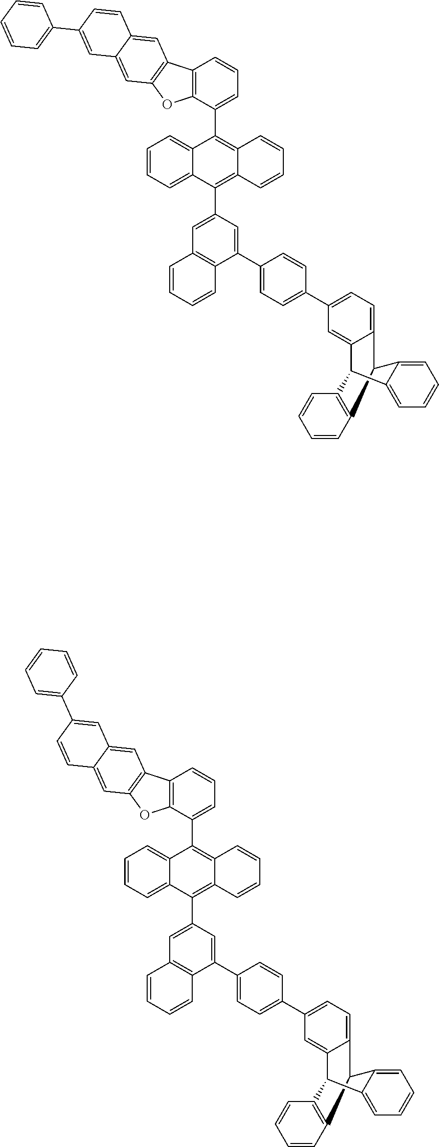 [SMILES: c1ccc(-c2ccc3cc4c(cc3c2)oc2c(-c3c5ccccc5c(-c5cc(-c6ccc(-c7ccc8c(c7)[C@H]7c9ccccc9[C@@H]8c8ccccc87)cc6)c6ccccc6c5)c5ccccc35)cccc24)cc1 ; c1ccc(-c2ccc3cc4oc5c(-c6c7ccccc7c(-c7cc(-c8ccc(-c9ccc%10c(c9)[C@H]9c%11ccccc%11[C@@H]%10c%10ccccc%109)cc8)c8ccccc8c7)c7ccccc67)cccc5c4cc3c2)cc1]

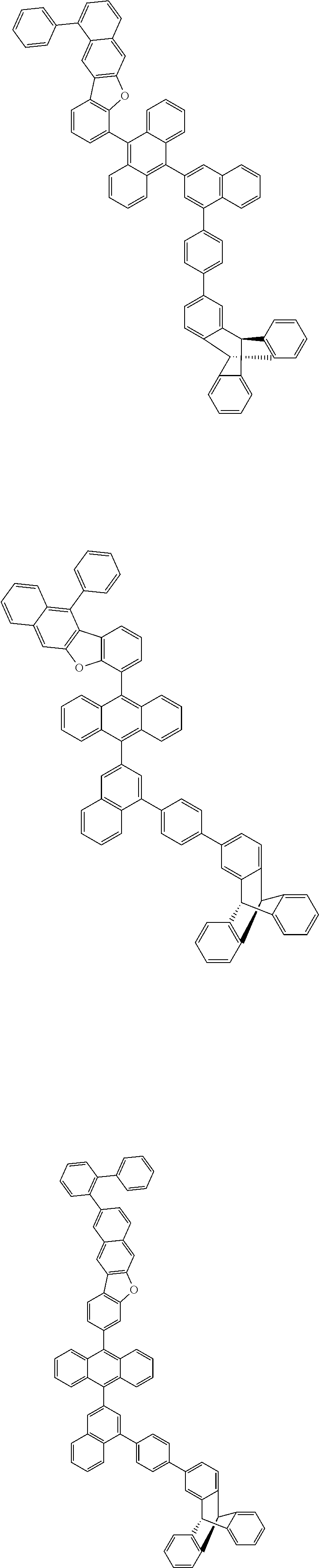 [SMILES: c1ccc(-c2cccc3cc4oc5c(-c6c7ccccc7c(-c7cc(-c8ccc(-c9ccc%10c(c9)[C@H]9c%11ccccc%11[C@@H]%10c%10ccccc%109)cc8)c8ccccc8c7)c7ccccc67)cccc5c4cc23)cc1 ; c1ccc(-c2c3ccccc3cc3oc4c(-c5c6ccccc6c(-c6cc(-c7ccc(-c8ccc9c(c8)[C@H]8c%10ccccc%10[C@@H]9c9ccccc98)cc7)c7ccccc7c6)c6ccccc56)cccc4c23)cc1 ; c1ccc(-c2ccccc2-c2ccc3cc4oc5cc(-c6c7ccccc7c(-c7cc(-c8ccc(-c9ccc%10c(c9)[C@H]9c%11ccccc%11[C@@H]%10c%10ccccc%109)cc8)c8ccccc8c7)c7ccccc67)ccc5c4cc3c2)cc1]

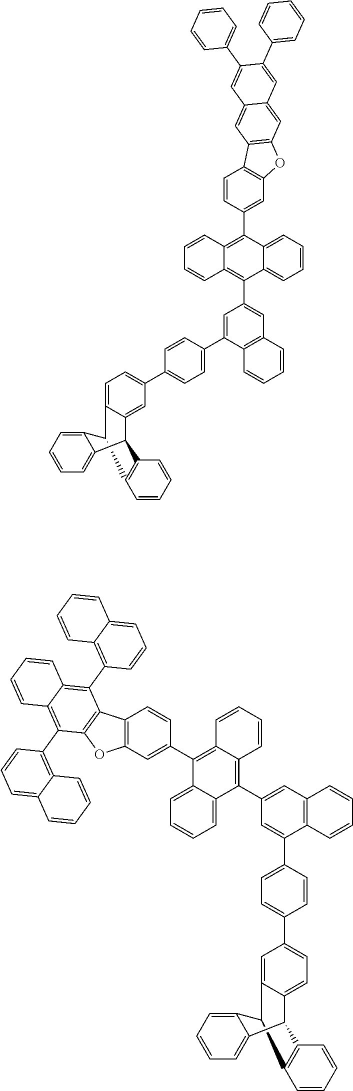 [SMILES: c1ccc(-c2cc3cc4oc5cc(-c6c7ccccc7c(-c7cc(-c8ccc(-c9ccc%10c(c9)[C@H]9c%11ccccc%11[C@@H]%10c%10ccccc%109)cc8)c8ccccc8c7)c7ccccc67)ccc5c4cc3cc2-c2ccccc2)cc1 ; c1ccc2c(-c3ccc(-c4ccc5c(c4)[C@H]4c6ccccc6[C@@H]5c5ccccc54)cc3)cc(-c3c4ccccc4c(-c4ccc5c(c4)oc4c(-c6cccc7ccccc67)c6ccccc6c(-c6cccc7ccccc67)c45)c4ccccc34)cc2c1]

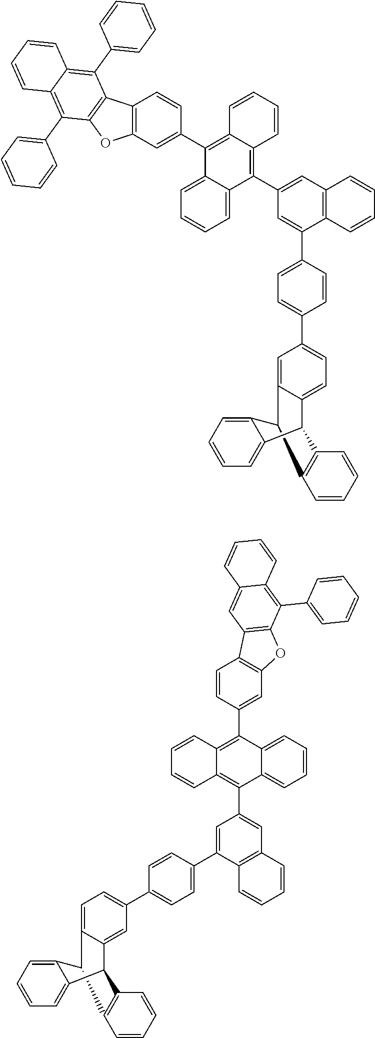 [SMILES: c1ccc(-c2c3ccccc3c(-c3ccccc3)c3c2oc2cc(-c4c5ccccc5c(-c5cc(-c6ccc(-c7ccc8c(c7)[C@H]7c9ccccc9[C@@H]8c8ccccc87)cc6)c6ccccc6c5)c5ccccc45)ccc23)cc1 ; c1ccc(-c2c3ccccc3cc3c2oc2cc(-c4c5ccccc5c(-c5cc(-c6ccc(-c7ccc8c(c7)[C@H]7c9ccccc9[C@@H]8c8ccccc87)cc6)c6ccccc6c5)c5ccccc45)ccc23)cc1]

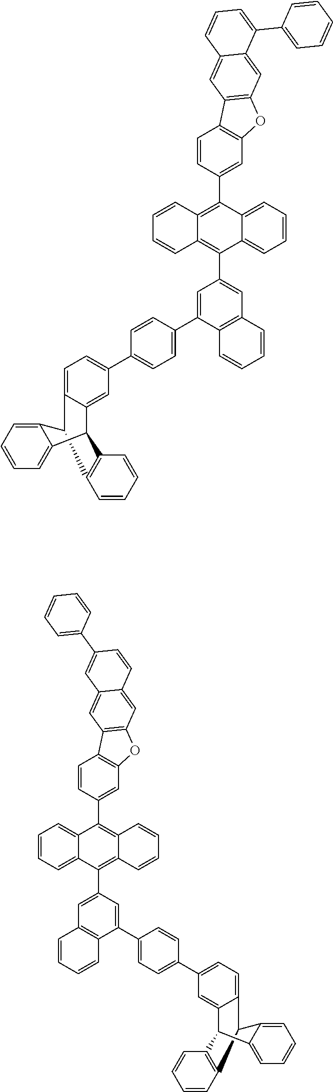 [SMILES: c1ccc(-c2cccc3cc4c(cc23)oc2cc(-c3c5ccccc5c(-c5cc(-c6ccc(-c7ccc8c(c7)[C@H]7c9ccccc9[C@@H]8c8ccccc87)cc6)c6ccccc6c5)c5ccccc35)ccc24)cc1 ; c1ccc(-c2ccc3cc4oc5cc(-c6c7ccccc7c(-c7cc(-c8ccc(-c9ccc%10c(c9)[C@H]9c%11ccccc%11[C@@H]%10c%10ccccc%109)cc8)c8ccccc8c7)c7ccccc67)ccc5c4cc3c2)cc1]

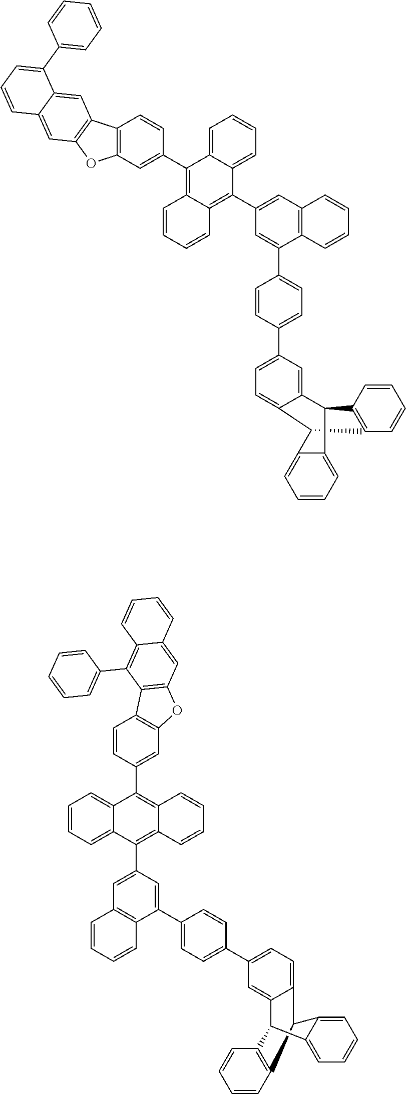 [SMILES: c1ccc(-c2cccc3cc4oc5cc(-c6c7ccccc7c(-c7cc(-c8ccc(-c9ccc%10c(c9)[C@H]9c%11ccccc%11[C@@H]%10c%10ccccc%109)cc8)c8ccccc8c7)c7ccccc67)ccc5c4cc23)cc1 ; c1ccc(-c2c3ccccc3cc3oc4cc(-c5c6ccccc6c(-c6cc(-c7ccc(-c8ccc9c(c8)[C@H]8c%10ccccc%10[C@@H]9c9ccccc98)cc7)c7ccccc7c6)c6ccccc56)ccc4c23)cc1]

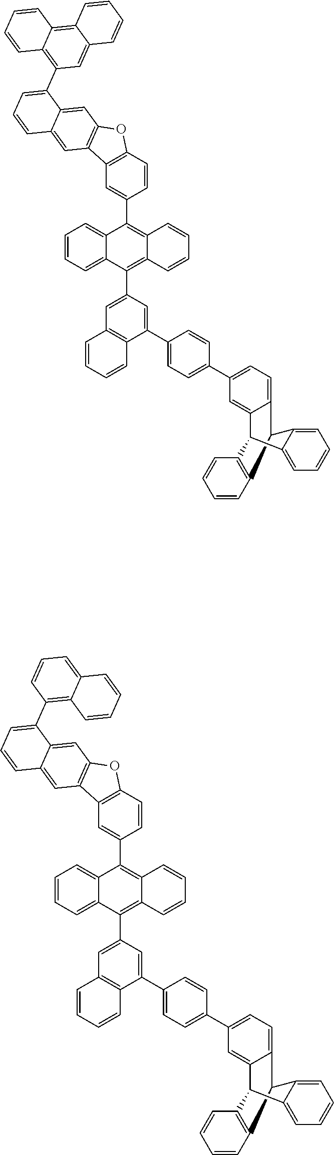 [SMILES: c1ccc2c(-c3ccc(-c4ccc5c(c4)[C@H]4c6ccccc6[C@@H]5c5ccccc54)cc3)cc(-c3c4ccccc4c(-c4ccc5oc6cc7c(-c8cc9ccccc9c9ccccc89)cccc7cc6c5c4)c4ccccc34)cc2c1 ; c1ccc2c(-c3ccc(-c4ccc5c(c4)[C@H]4c6ccccc6[C@@H]5c5ccccc54)cc3)cc(-c3c4ccccc4c(-c4ccc5oc6cc7c(-c8cccc9ccccc89)cccc7cc6c5c4)c4ccccc34)cc2c1]

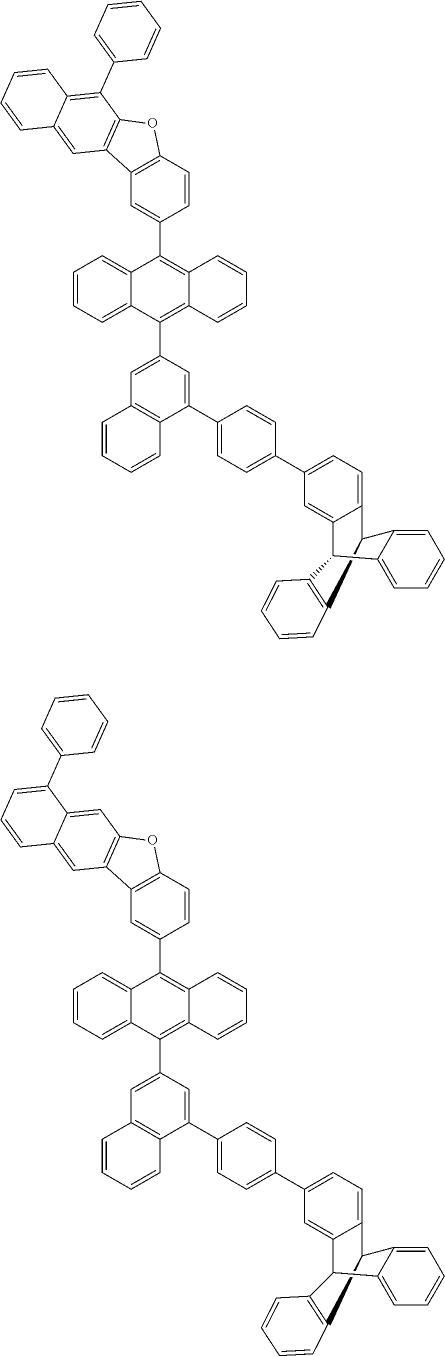

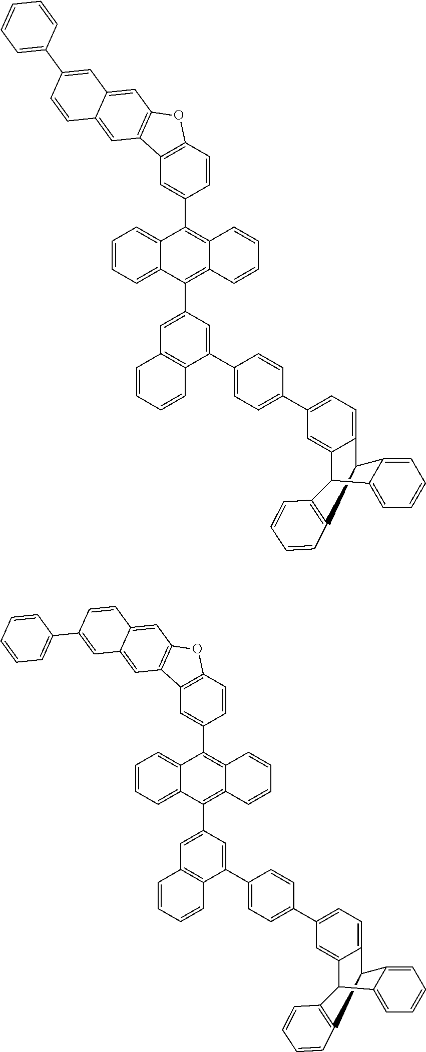

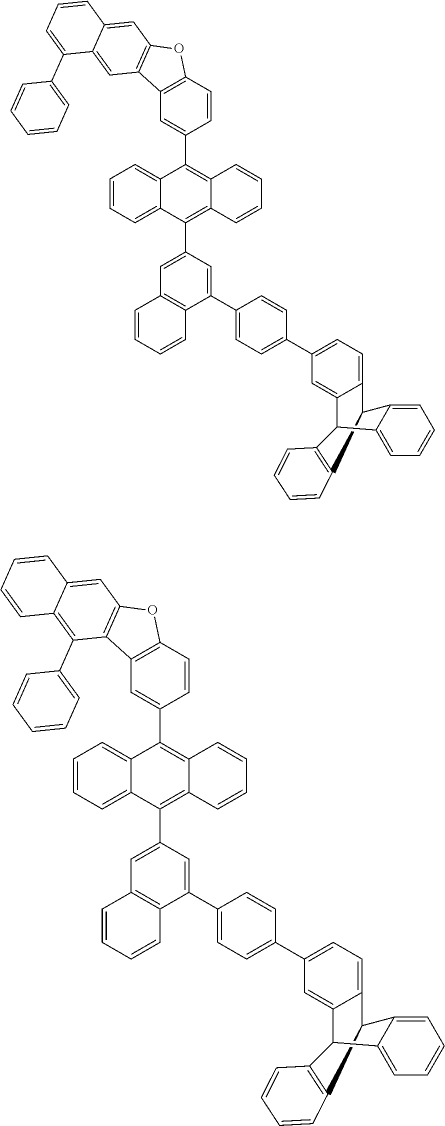

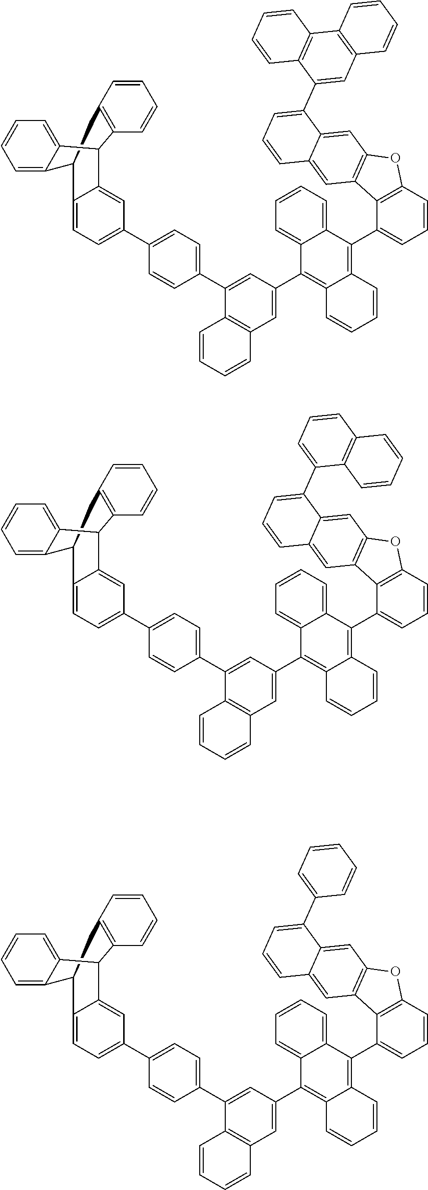

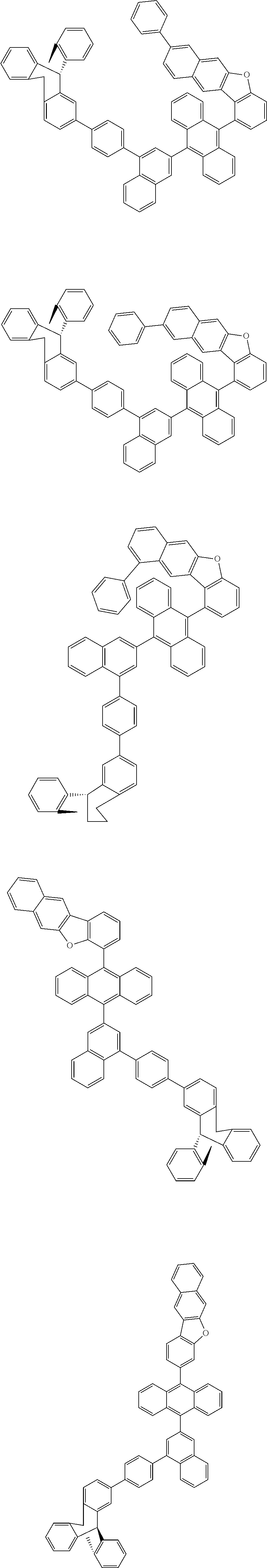

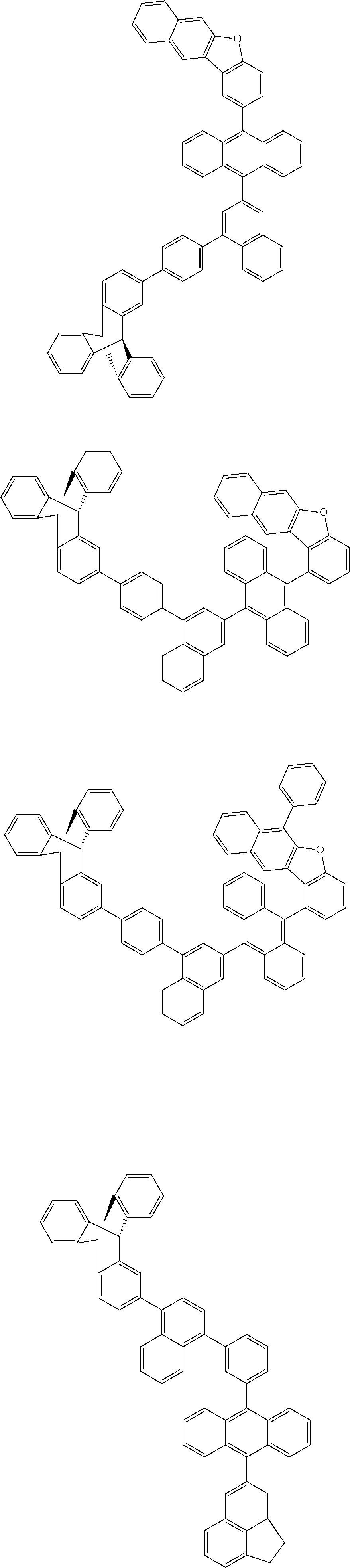

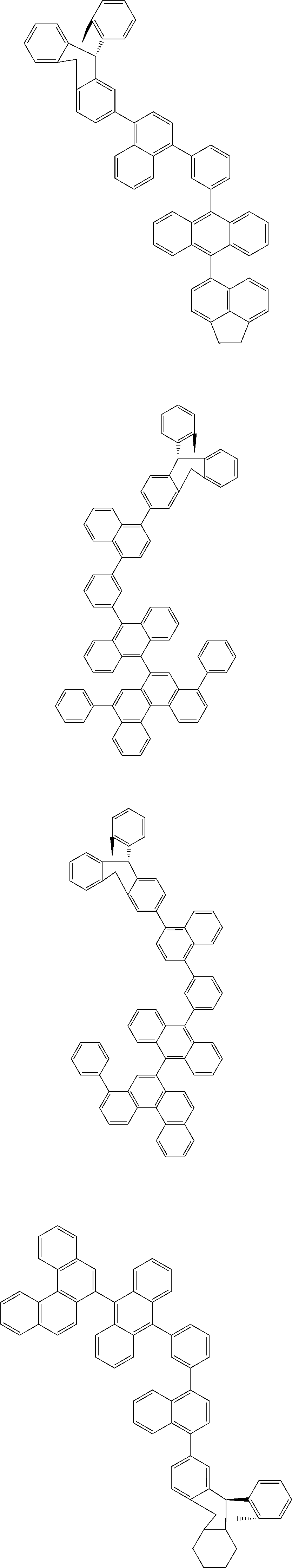

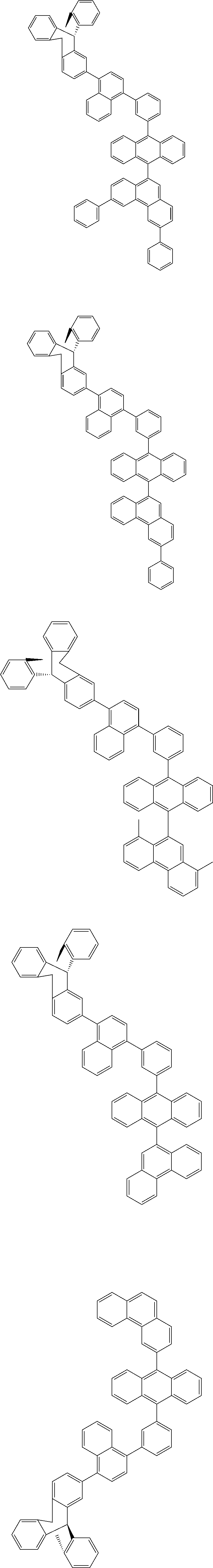

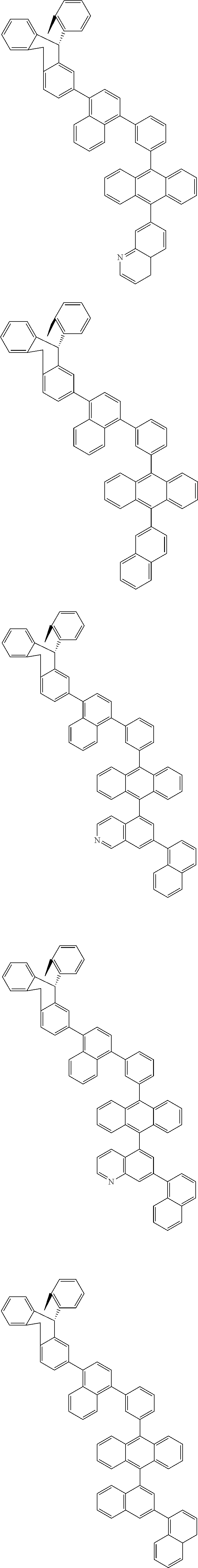

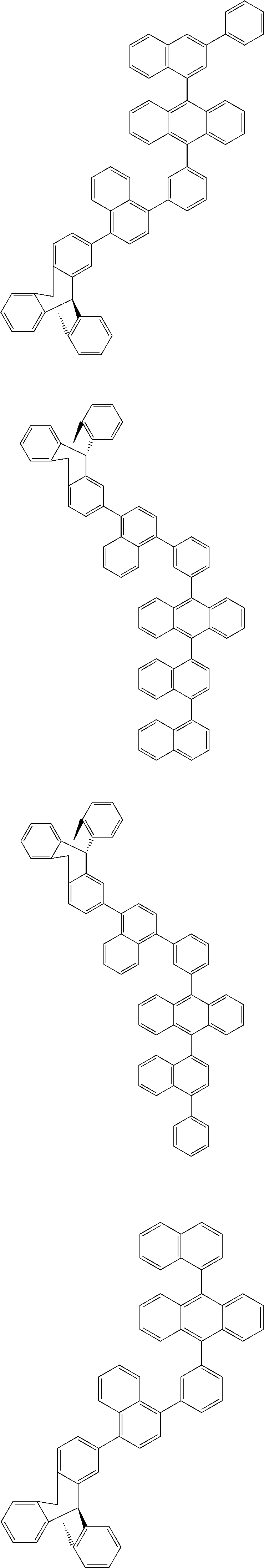

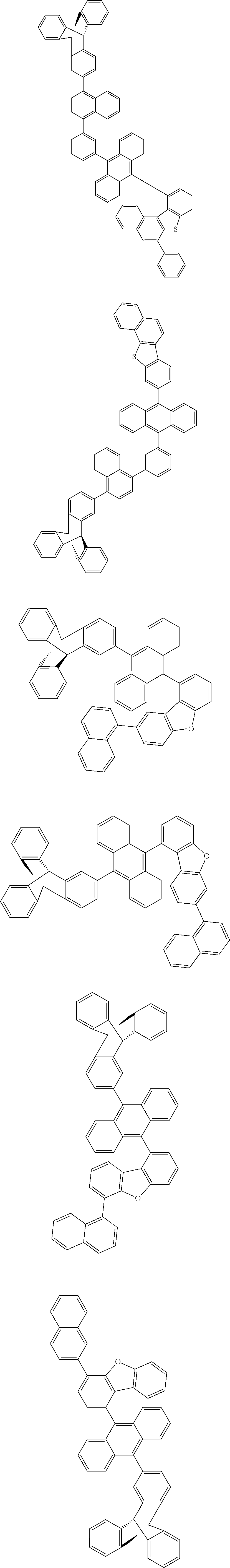

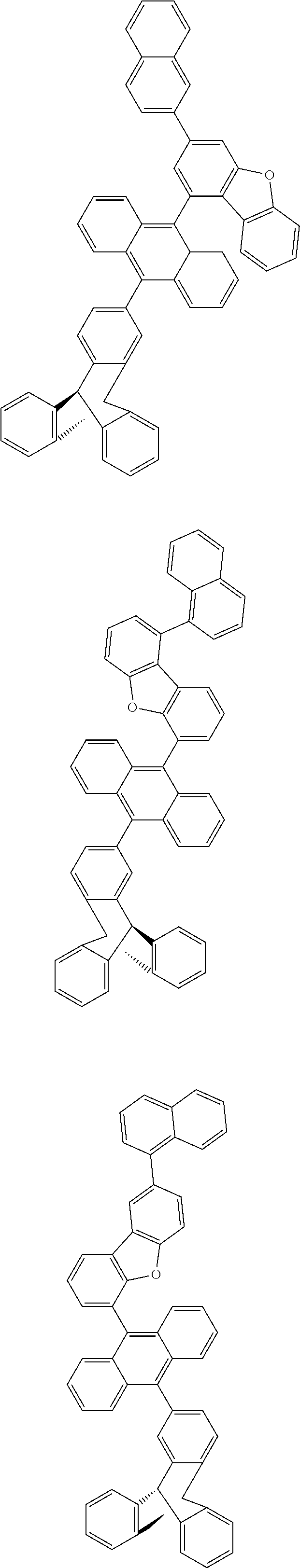

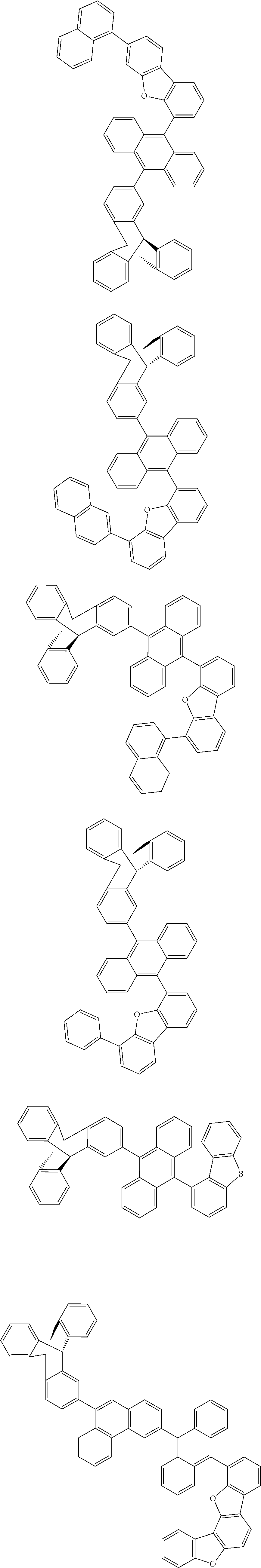

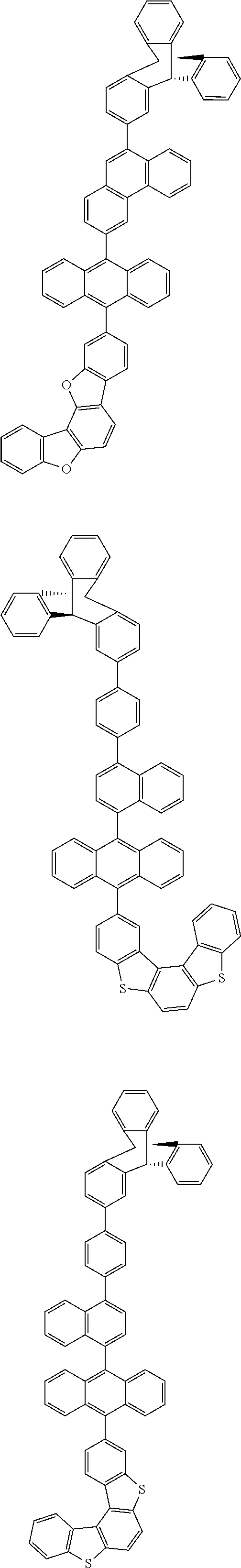

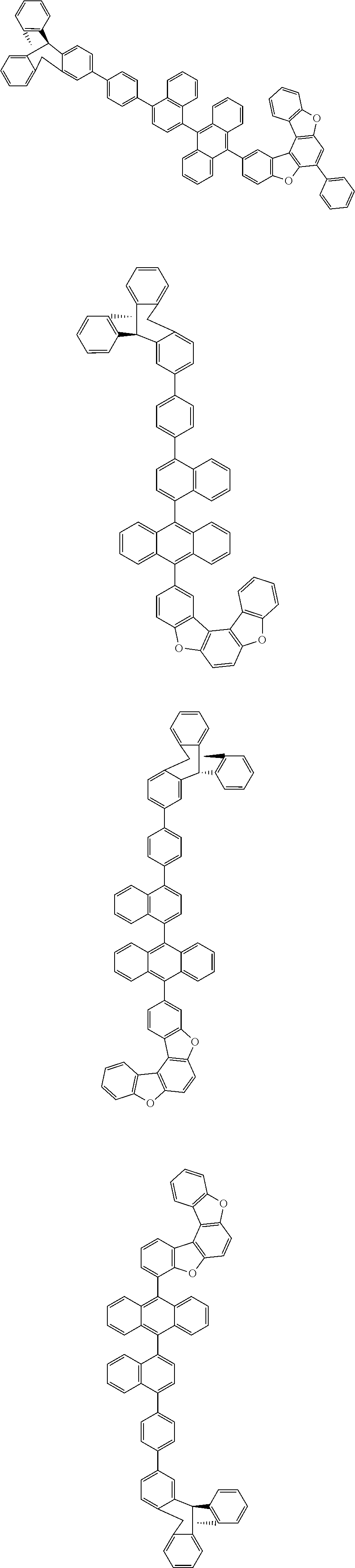

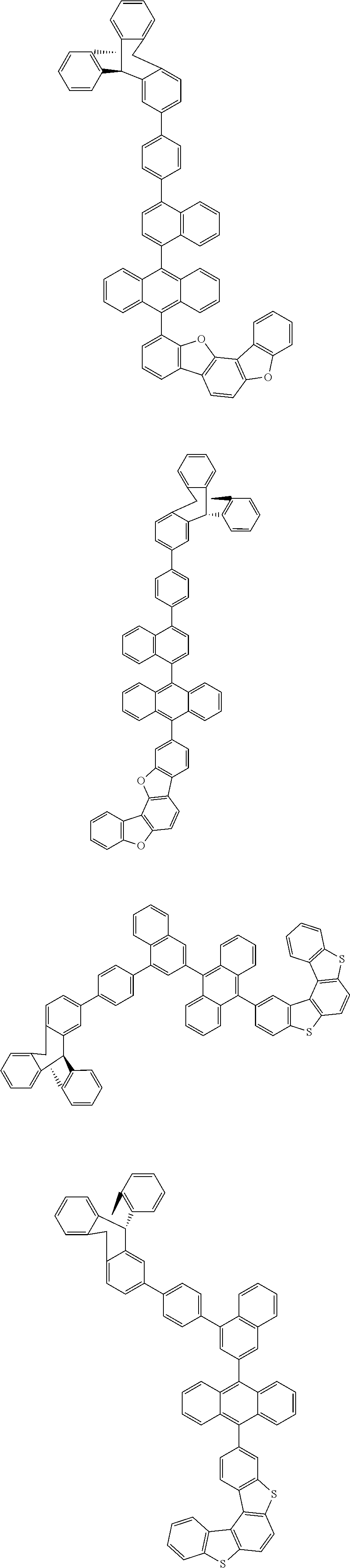

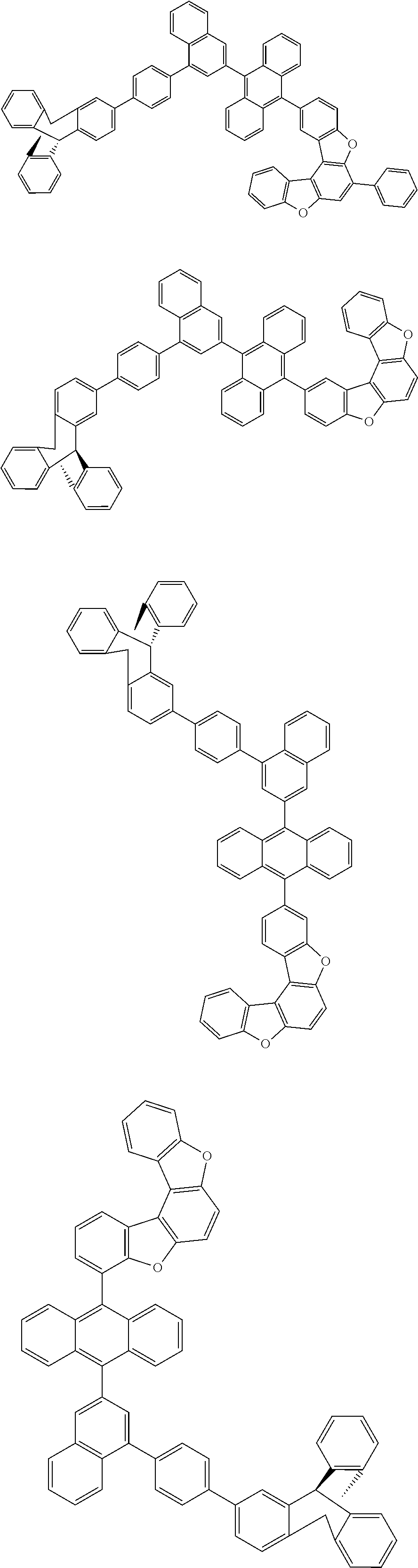

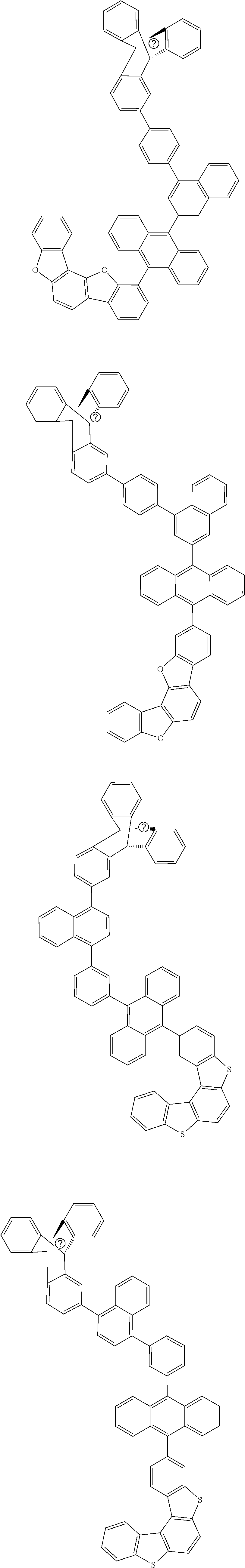

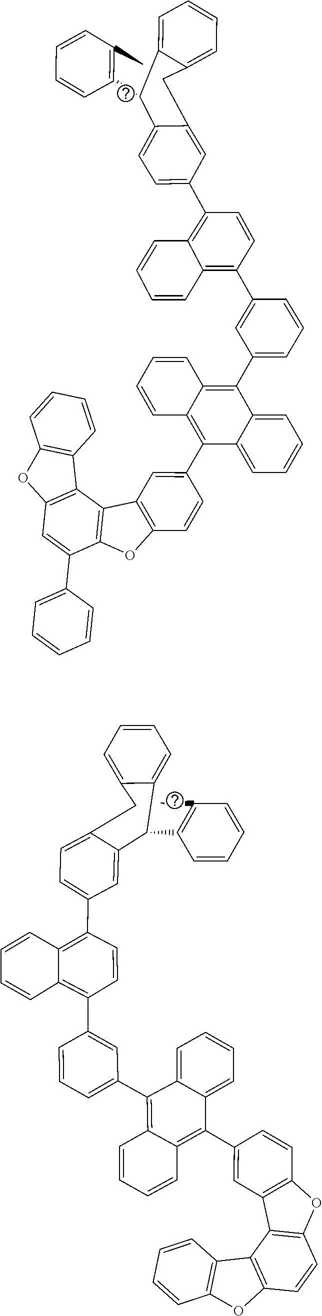

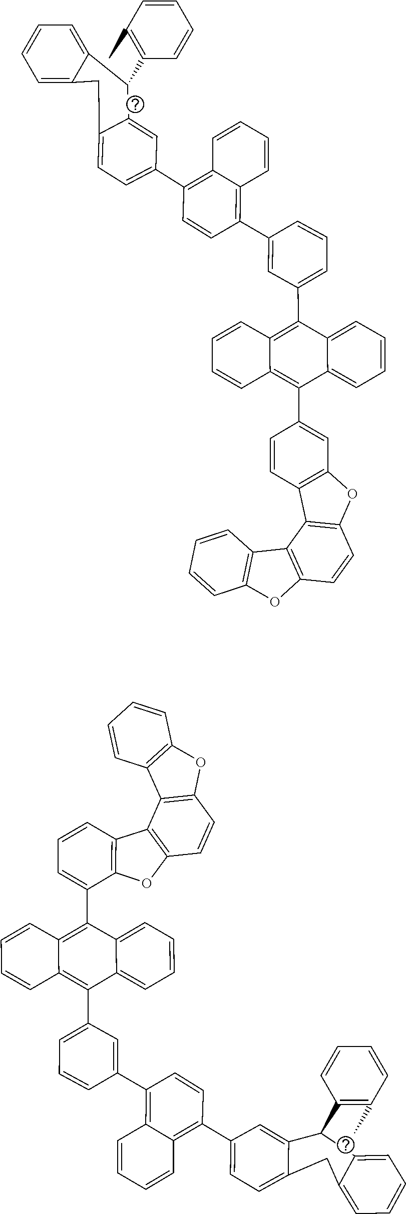

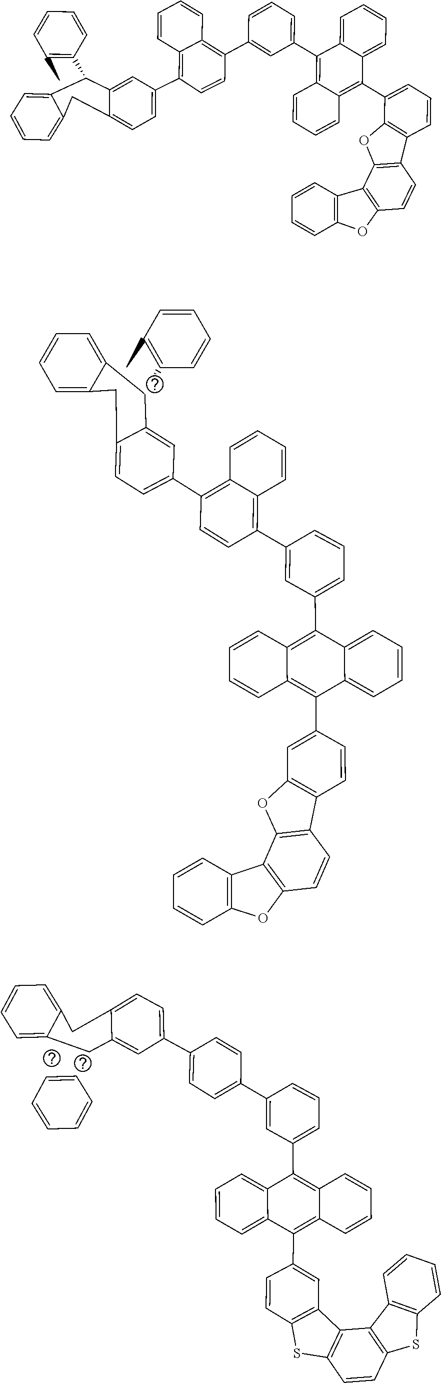

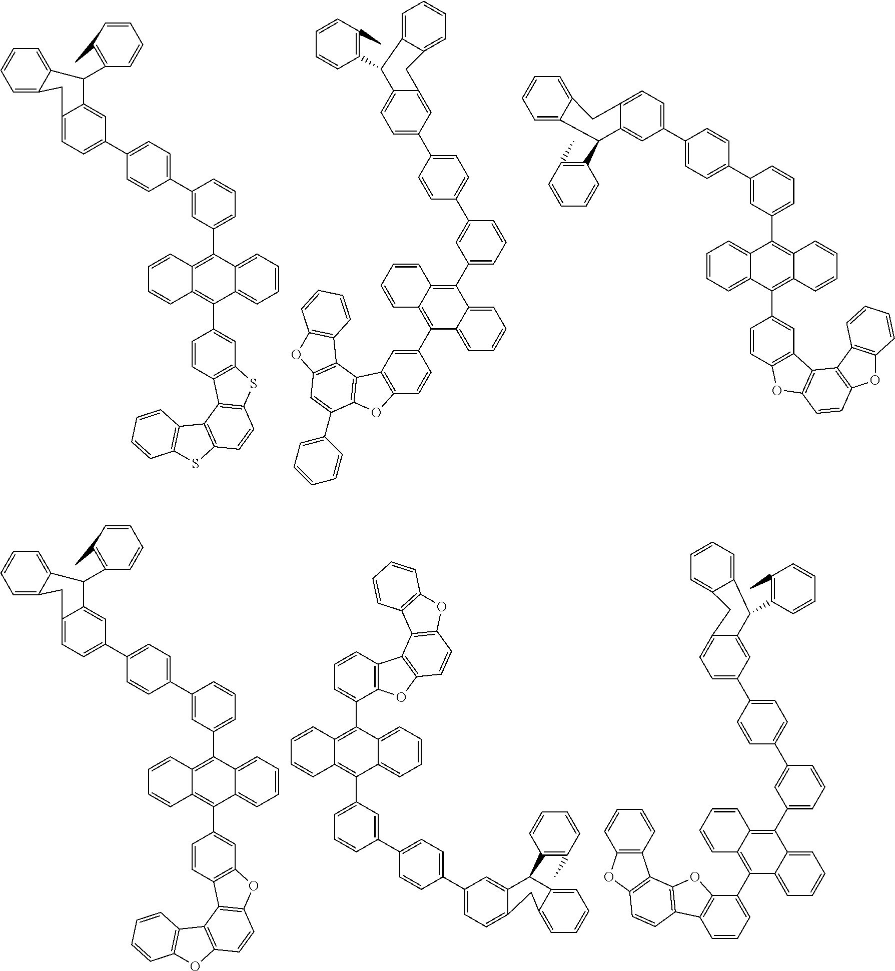

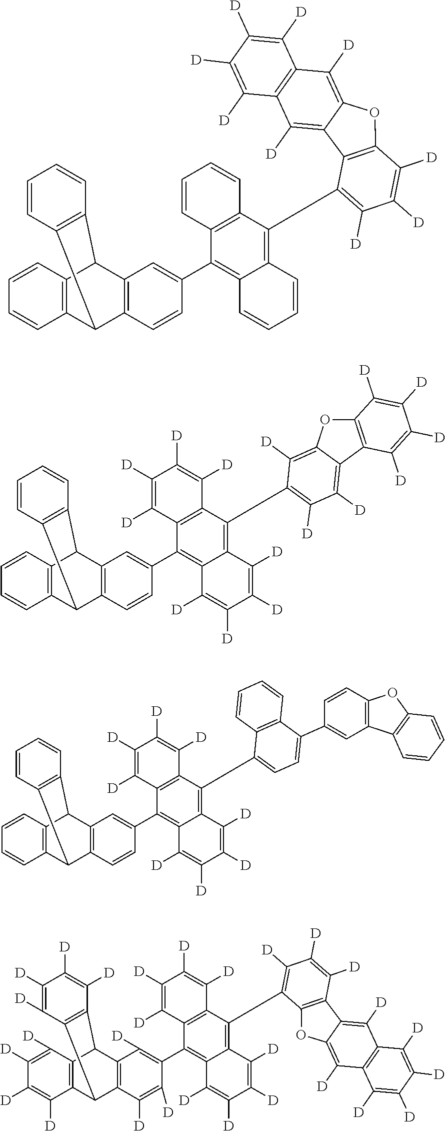

The present disclosure also provides an organic electroluminescent device including an anode, an organic light-emitting layer, and a cathode sequentially stacked, wherein the organic light-emitting layer contains the aforementioned compound.

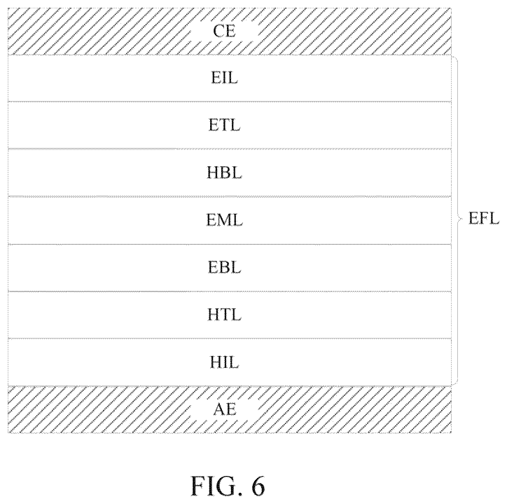

As shown in FIG. 6, the electroluminescent device is a thin-film electroluminescent device including a first electrode, a light-emitting functional layer EFL, and a second electrode sequentially stacked. The first electrode and second electrode of the device can respectively provide different charge carriers to the light-emitting function layer EFL, which recombine in the light-emitting function layer EFL to form excitons and emit light. In embodiments of the present disclosure, the second electrode may be a transparent or semi-transparent electrode capable of light emission, allowing light generated in the light-emitting functional layer EFL to exit through the second electrode. In one example, the first electrode is a reflective electrode, which can further improve the light extraction efficiency of the device.

In embodiments of the present disclosure, one of the first electrode and second electrode is the anode (AE), and the other is the cathode (CE). The anode can inject hole carriers into the light-emitting functional layer EFL, while the cathode can inject electron carriers into the light-emitting functional layer EFL. In one example, the first electrode is the anode, and the second electrode is the cathode.

As shown in FIG. 6, the electroluminescent device is an OLED (organic light-emitting diode), and the light-emitting functional layer EFL may include an organic light-emitting layer EML as the light-emitting layer, as well as one or more of a hole injection layer HIL, a hole transport layer HTL, an electron blocking layer EBL, a hole blocking layer HBL, an electron transport layer ETL, and an electron injection layer EIL.

In one example, the organic light-emitting layer EML may include a blue host material and a light-emitting guest material, wherein the guest material may be a fluorescent dopant, and the blue host material is the compound provided in the embodiments of the present disclosure.

In one example, the electroluminescent device includes an anode (as the first electrode), a hole injection layer, a hole transport layer, an electron blocking layer, an organic light-emitting layer (including host material and guest dopant material), a hole blocking layer, electron transport layer, an electron injection layer, and a cathode sequentially stacked.

Optionally, the hole injection layer may be selected from, but not limited to, inorganic oxides, p-type dopants with strong electron-withdrawing systems, or dopants of hole transport materials, such as hexacyanohexaazatriphenylene, 2,3,5,6-tetrafluoro-7,7,8,8-tetracyanoquinodimethane (F4TCNQ), 1,2,3-tris[(cyano)(4-cyano-2,3,5,6-tetrafluorophenyl)methylene]cyclopropane, and the like. Inorganic oxides include, but are not limited to, molybdenum oxide, titanium oxide, vanadium oxide, rhenium oxide, ruthenium oxide, chromium oxide, zirconium oxide, hafnium oxide, tantalum oxide, silver oxide, tungsten oxide, manganese oxide, and the like. The hole injection layer may also be formed by p-type doping of hole transport materials, with a thickness of 5 nm to 30 nm through co-evaporation.

Optionally, the hole transport layer may include arylamine or carbazole-based materials, such as NPB, TPD, BAFLP, or DFLDPBi.

Optionally, the thickness of the hole transport layer HTL ranges from 100 to 2000 nm.

Optionally, the light-emitting auxiliary layer also exhibits excellent hole transport properties and may serve as a blue light-emitting auxiliary layer. Its materials may similarly be arylamine or carbazole-based, such as CBP or PCzPA.

Optionally, the thickness of the light-emitting auxiliary layer ranges from 5 to 100 nm.

Optionally, the blue light guest material may be pyrene derivatives, fluorene derivatives, perylene derivatives, styrylamine derivatives, metal complexes, and the like, such as TBPe, BDAVBi, DPAVBi, or FIrpic.

Optionally, the hole blocking layer and electron transport layer generally include aromatic heterocyclic compounds, which may be selected from, but not limited to, benzimidazoles, triazines, pyrimidines, pyridines, pyrazines, quinoxalines, quinolines, oxadiazoles, diazaphospholes, phosphine oxides, aromatic ketones, lactams, boranes, and their derivatives, either individually or in combinations of two or more. Examples include OXD-7, TAZ, p-EtTAZ, BPhen, BCP and the like.

Optionally, the thickness of the hole blocking layer may range from 5 to 100 nm.

Optionally, the thickness of the electron transport layer may range from 20 to 100 nm.

Optionally, the electron injection layer typically includes an alkali metal or other metal, such as LiF, Yb, Mg, Ca, or a compound thereof. The thickness of the electron injection layer may range from 1 to 10 nm.

Optionally, the organic electroluminescent device may be formed on a substrate. In one example, the substrate may be a transparent rigid or flexible material, such as glass or polyimide.

Optionally, the anode may be a high-work-function electrode material, such as transparent oxides (ITO, IZO) or composite electrodes like ITO/Ag/ITO, Ag/IZO, CNT/ITO, CNT/IZO, GO/ITO, or GO/IZO.

The present disclosure also provides a display apparatus including any of the organic electroluminescent devices described in the above embodiments. The display apparatus may be a smartphone screen, smartwatch screen, or other types of display devices. Since the display apparatus incorporates the organic electroluminescent devices described in the embodiments above, it achieves the same beneficial effects, which will not be reiterated here.

### Synthetic Route

The compounds provided in the present disclosure can be synthesized via the following synthetic route:

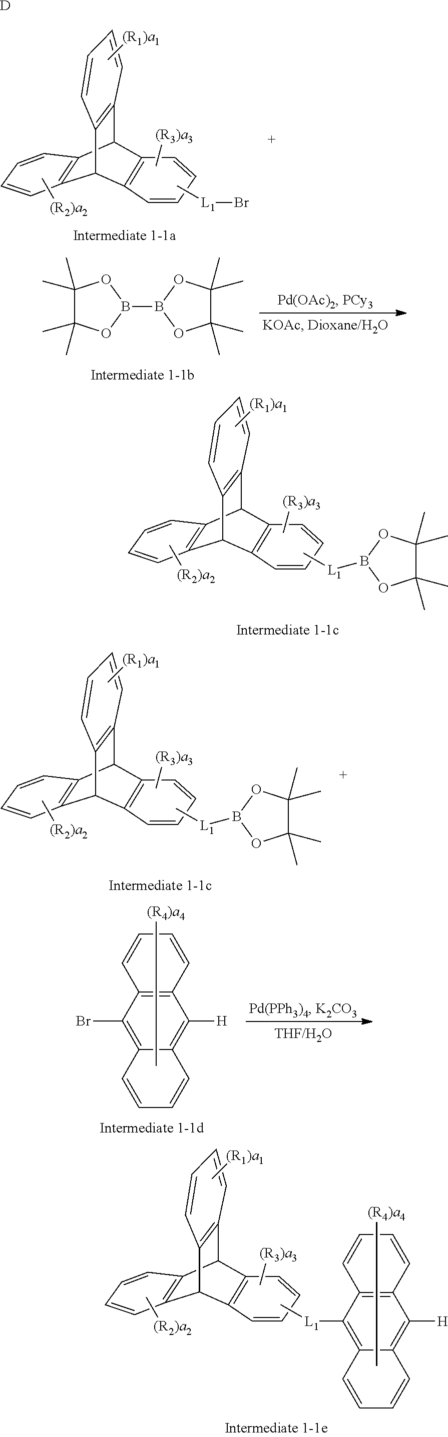

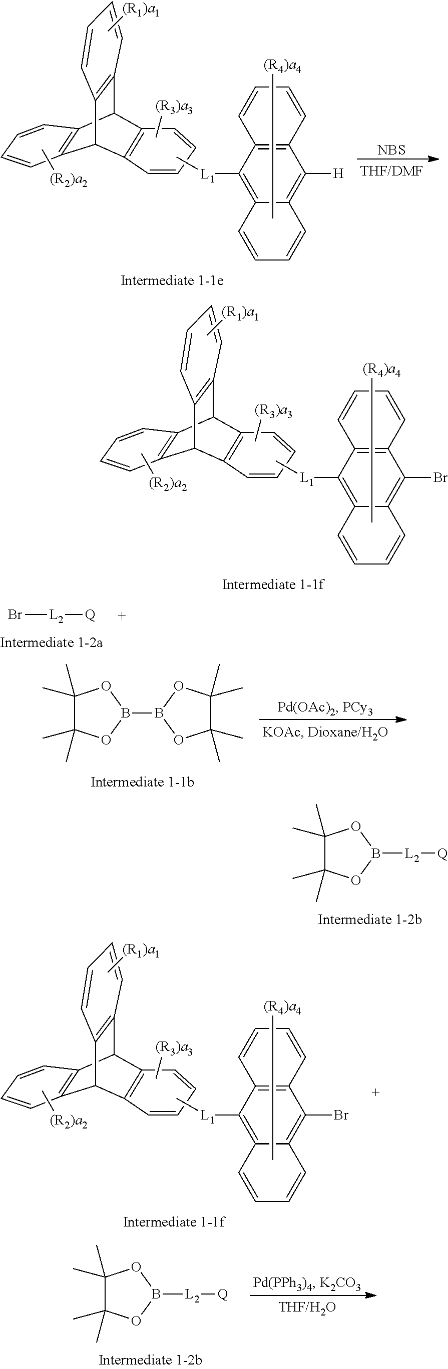

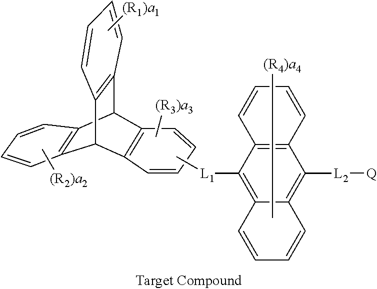

As shown in the above reaction route, Intermediate 1-1a reacts with Intermediate 1-1b to form Intermediate 1-1c; Intermediate 1-1c reacts with Intermediate 1-1d to generate Intermediate 1-1e; Intermediate 1-1e undergoes bromination to yield Intermediate 1-1f; Intermediate 1-1f couples with Intermediate 1-2b to produce the target compound. Intermediate 1-2b is obtained by reacting Intermediate 1-2a with bis(pinacolato)diboron. Specific reaction conditions for each step can be found in the detailed examples below.

### Specific Synthetic Examples

### Synthetic of Intermediate 1-3

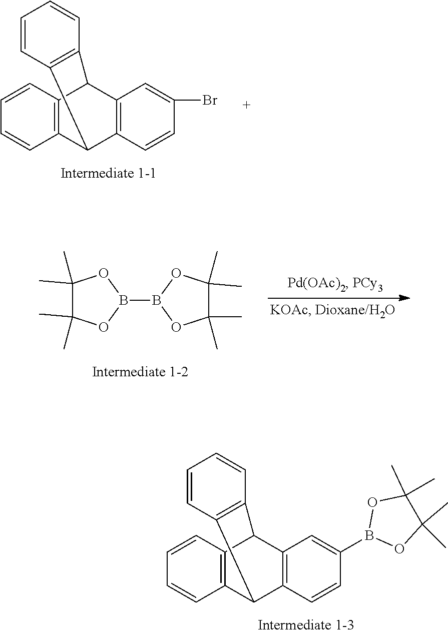

In a three-necked flask, Intermediate 1-1 (25 mmol), Intermediate 1-2 (25 mmol), Pd(OAc)₂ (1.25 mmol), (PCy₃) (2.5 mmol), and KOAc (125 mmol) were added to dioxane, refluxed and stirred for 12 hours. After the reaction, the mixture was extracted with toluene, washed with H₂O, and then recrystallized with hexane to afford Intermediate 1-3 in 74% yield.

### Synthetic of Intermediate 1-5

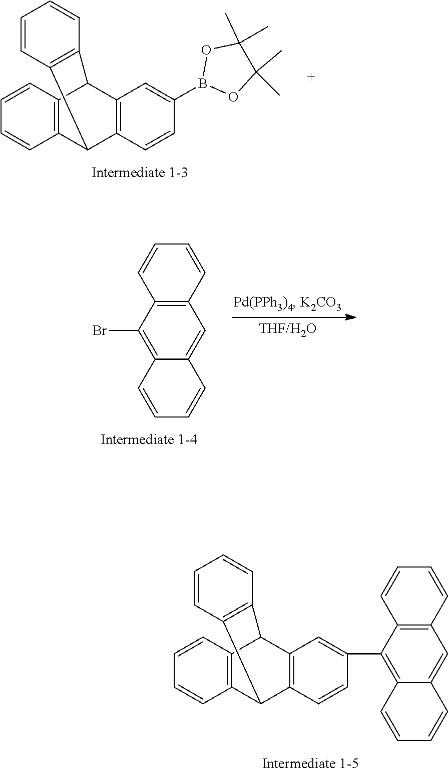

Under nitrogen atmosphere, Intermediate 1-3 (60 mmol) and Intermediate 1-4 (50 mmol) in a 1.2:1 molar ratio, potassium carbonate (150 mmol), and Pd(PPh₃)₄ (5 mmol) were dissolved in a mixed solvent of THF and water (300 ml, 3:1 v/v). The reaction mixture was heated to 90° C. and stirred for 12 hours. After cooling to room temperature, the resulting solid was collected by filtration. The solid was sequentially washed with THF (100 ml), ethyl acetate (500 ml), water (500 ml), and ethanol (300 ml). Drying afforded Intermediate 1-5 in 86.8% yield.

### Synthetic of Intermediate 1-6

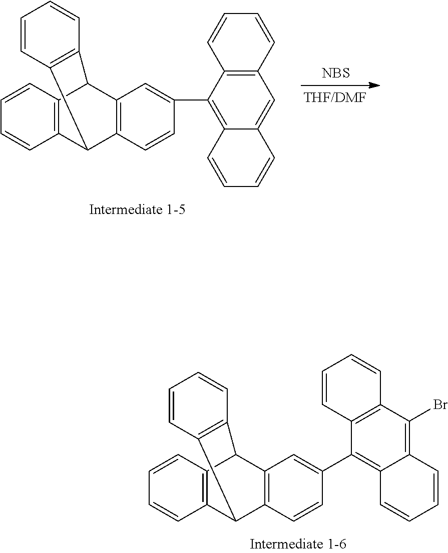

Intermediate 1-5 (46 mmol) was dissolved in THE (500 mL), followed by addition of NBS (50.7 mmol) dissolved in DMF. The mixture was stirred at room temperature for 12 hours. After completion of the reaction, the resulting solid was filtered, washed with distilled water, and dried to afford Intermediate 1-6 in 77.56% yield.

### Synthetic of Compound BH-1

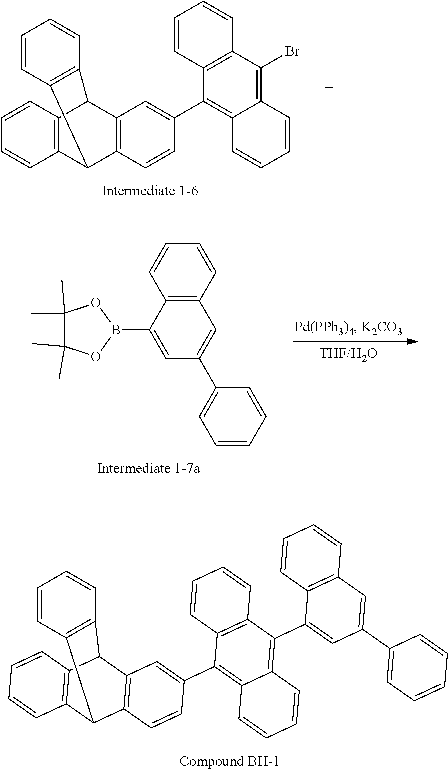

Referring to the synthesis method of Intermediate 1-3, 1-bromo-3-phenylnaphthalene was reacted with bis(pinacolato)diboron to generate Intermediate 1-7a.

Referring to the synthesis method of Intermediate 1-5, Intermediate 1-6 was coupled with Intermediate 1-7a to produce Compound BH-1 with a yield of 80.3%.

The 1H NMR (400 MHz, DMSO) characterization of Compound BH-1 is as follows: δ 9.01 (s, 1H), 8.23 (d, J=12.9 Hz, 5H), 8.06 (s, 1H), 7.94 (s, 1H), 7.86 (s, 1H), 7.74 (d, J=8.1 Hz, 3H), 7.61 (s, 1H), 7.56-7.26 (m, 13H), 7.09 (s, 4H), 4.90 (d, J=2.3 Hz, 2H). The mass spectrometry (MS) characterization of Compound BH-1 is m/z: 632.25.

### Synthetic of Compound BH-2

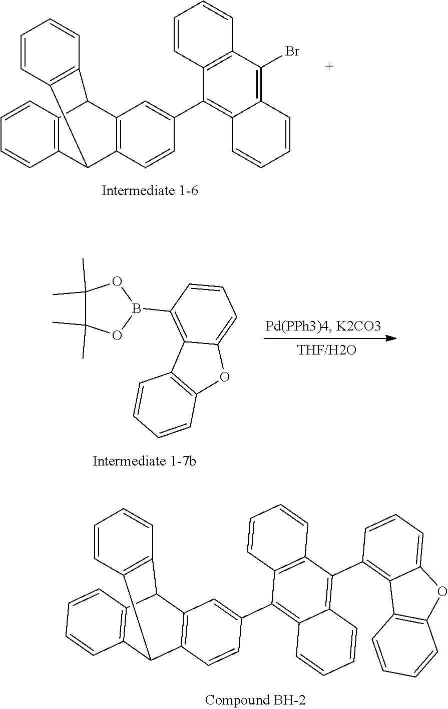 [SMILES: Brc1c2ccccc2c(-c2ccc3c(c2)C2c4ccccc4C3c3ccccc32)c2ccccc12 ; CC1(C)OB(c2cccc3oc4ccccc4c23)OC1(C)C ; c1ccc2c(c1)C1c3ccccc3C2c2cc(-c3c4ccccc4c(-c4cccc5oc6ccccc6c45)c4ccccc34)ccc21]

Intermediate 1-7b was prepared by referring to the preparation method of Intermediate 1-7a.

By referring to the synthesis method of Compound BH-1, Intermediate 1-6 was coupled with Intermediate 1-7b to produce Compound BH-2 with a yield of 88.8%.

The 1H NMR (400 MHz, DMSO) characterization of Compound BH-2 is as follows: δ 8.21 (s, 4H), 7.98 (s, 1H), 7.89 (s, 1H), 7.82 (s, 1H), 7.74 (s, 1H), 7.69 (s, 1H), 7.66-7.49 (m, 3H), 7.46-7.35 (m, 9H), 7.31 (s, 1H), 7.09 (s, 4H), 4.85 (d, J=5.2 Hz, 2H). The mass spectrometry (MS) characterization of Compound BH-2 is m/z: 596.21.

### Synthetic of Compound BH-3

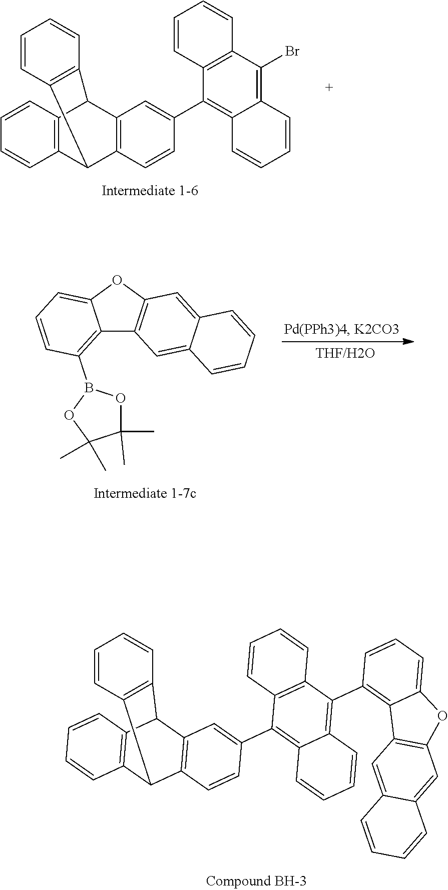 [SMILES: Brc1c2ccccc2c(-c2ccc3c(c2)C2c4ccccc4C3c3ccccc32)c2ccccc12 ; CC1(C)OB(c2cccc3oc4cc5ccccc5cc4c23)OC1(C)C ; c1ccc2c(c1)C1c3ccccc3C2c2cc(-c3c4ccccc4c(-c4cccc5oc6cc7ccccc7cc6c45)c4ccccc34)ccc21]

Intermediate 1-7c was prepared by referring to the preparation method of Intermediate 1-7a.

By referring to the synthesis method of Compound BH-1, Intermediate 1-6 was coupled with Intermediate 1-7c to produce Compound BH-3 with a yield of 87.2%.

The 1H NMR (400 MHz, DMSO) characterization of Compound BH-3 is as follows: δ 8.35-8.04 (m, 6H), 7.91 (s, 1H), 7.86-7.50 (m, 9H), 7.40 (t, J=4.6 Hz, 8H), 7.09 (s, 4H), 4.87 (d, J=4.2 Hz, 2H). The mass spectrometry (MS) characterization of Compound BH-3 is m/z: 646.23.

### Synthetic of Compound BH-4

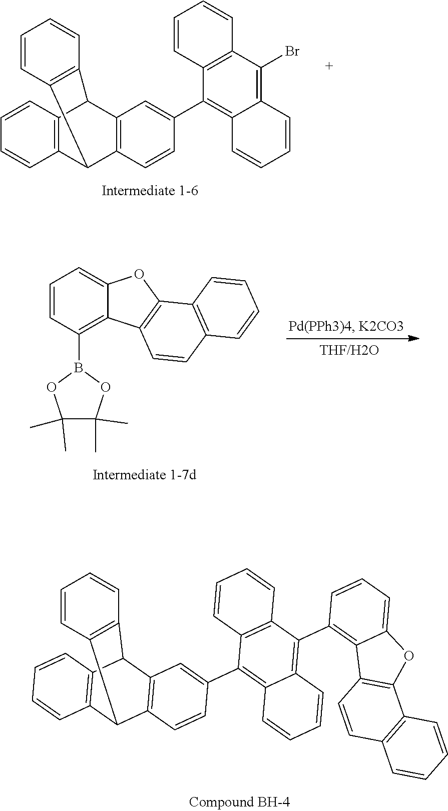 [SMILES: Brc1c2ccccc2c(-c2ccc3c(c2)C2c4ccccc4C3c3ccccc32)c2ccccc12 ; CC1(C)OB(c2cccc3oc4c5ccccc5ccc4c23)OC1(C)C ; c1ccc2c(c1)C1c3ccccc3C2c2cc(-c3c4ccccc4c(-c4cccc5oc6c7ccccc7ccc6c45)c4ccccc34)ccc21]

Intermediate 1-7d was prepared by referring to the preparation method of Intermediate 1-7a.

By referring to the synthesis method of Compound BH-1, Intermediate 1-6 was coupled with Intermediate 1-7d to produce Compound BH-4 with a yield of 84.2%.

The 1H NMR (400 MHz, DMSO) characterization of Compound BH-4 is as follows: δ 8.22 (d, J=4.0 Hz, 5H), 8.11 (s, 1H), 7.89 (s, 1H), 7.88-7.73 (m, 3H), 7.64 (dd, J=31.0, 17.0 Hz, 5H), 7.52-7.32 (m, 9H), 7.09 (s, 4H), 4.93 (d, J=2.8 Hz, 2H). The mass spectrometry (MS) characterization of Compound BH-4 is m/z: 646.23.

### Synthetic of Compound BH-5

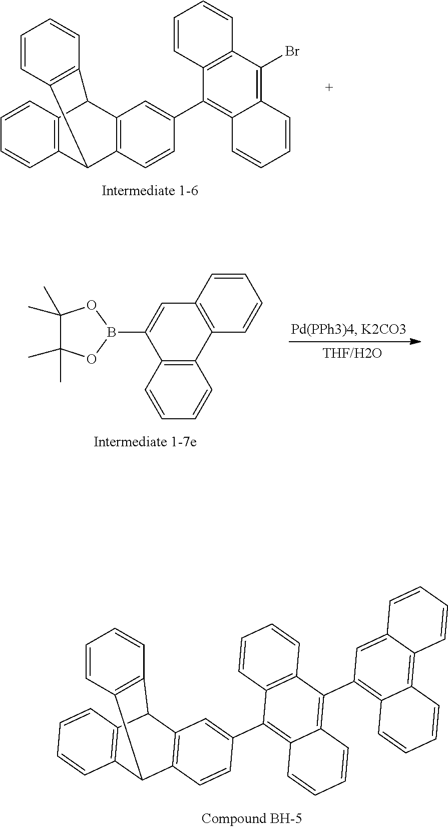

Intermediate 1-7e was prepared by referring to the preparation method of Intermediate 1-7a.

By referring to the synthesis method of Compound BH-1, Intermediate 1-6 was coupled with Intermediate 1-7e to produce Compound BH-5 with a yield of 83.9%.

The 1H NMR (400 MHz, DMSO) characterization of Compound BH-5 is as follows: δ 9.08 (s, 1H), 8.84 (s, 1H), 8.36 (s, 1H), 8.24 (d, J=24.0 Hz, 5H), 7.88 (d, J=14.5 Hz, 2H), 7.82-7.55 (m, 6H), 7.40 (t, J=3.2 Hz, 8H), 7.09 (s, 4H), 4.91 (d, J=2.4 Hz, 2H). The mass spectrometry (MS) characterization of Compound BH-5 is m/z: 606.23.

### Synthetic of Compound BH-6

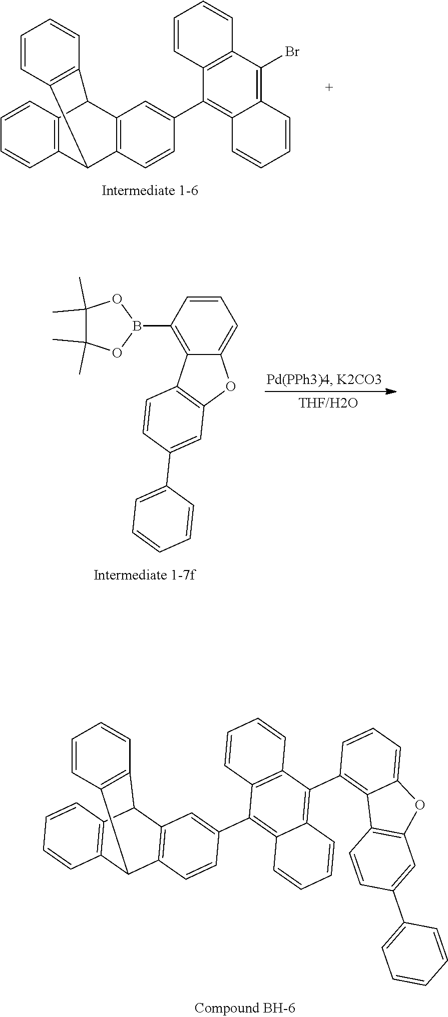 [SMILES: Brc1c2ccccc2c(-c2ccc3c(c2)C2c4ccccc4C3c3ccccc32)c2ccccc12 ; CC1(C)OB(c2cccc3oc4cc(-c5ccccc5)ccc4c23)OC1(C)C ; c1ccc(-c2ccc3c(c2)oc2cccc(-c4c5ccccc5c(-c5ccc6c(c5)C5c7ccccc7C6c6ccccc65)c5ccccc45)c23)cc1]

Intermediate 1-7f was prepared by referring to the preparation method of Intermediate 1-7a.

By referring to the synthesis method of Compound BH-1, Intermediate 1-6 was coupled with Intermediate 1-7f to produce Compound BH-6 with a yield of 86.4%.

The 1H NMR (400 MHz, DMSO) characterization of Compound BH-6 is as follows: δ 8.21 (s, 4H), 8.03 (s, 1H), 7.94 (s, 1H), 7.89 (s, 1H), 7.84 (s, 1H), 7.82 (s, 1H), 7.77 (s, 1H), 7.75 (d, J=1.0 Hz, 2H), 7.62 (s, 2H), 7.53 (m, 2H), 7.40 (dd, J=5.2, 2.8 Hz, 10H), 7.09 (s, 4H), 4.85 (d, J=3.8 Hz, 2H). The mass spectrometry (MS) characterization of Compound BH-6 is m/z: 672.25.

### Synthetic of Compound BH-7

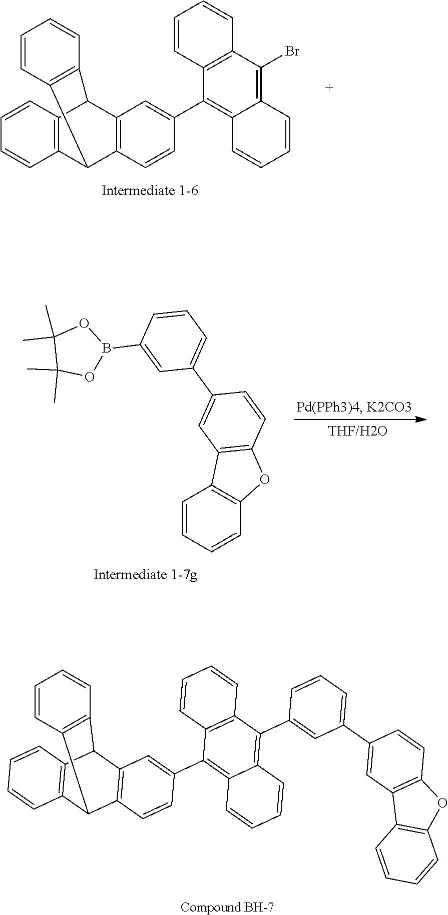 [SMILES: Brc1c2ccccc2c(-c2ccc3c(c2)C2c4ccccc4C3c3ccccc32)c2ccccc12 ; CC1(C)OB(c2cccc(-c3ccc4oc5ccccc5c4c3)c2)OC1(C)C ; c1cc(-c2ccc3oc4ccccc4c3c2)cc(-c2c3ccccc3c(-c3ccc4c(c3)C3c5ccccc5C4c4ccccc43)c3ccccc23)c1]

Intermediate 1-7g was prepared by referring to the preparation method of Intermediate 1-7a.

By referring to the synthesis method of Compound BH-1, Intermediate 1-6 was coupled with Intermediate 1-7g to produce Compound BH-7 with a yield of 87.67%.

The 1H NMR (400 MHz, DMSO) characterization of Compound BH-7 is as follows: δ 8.32-8.10 (m, 6H), 7.98 (s, 1H), 7.92-7.67 (m, 5H), 7.67-7.50 (m, 4H), 7.49-7.23 (m, 10H), 7.09 (s, 4H), 4.83 (d, J=3.7 Hz, 2H). The mass spectrometry (MS) characterization of Compound BH-7 is m/z: 672.25.

By referring to the synthesis method of Compound BH-1, compounds BH-8 to BH-17 in Table 1 and Table 2 were synthesized. For the synthesis of compounds BH-8 to BH-12, Intermediate 1 in Table 1 was used to replace Intermediate 1-6 in the synthesis method of Compound BH-1, and Intermediate 2 in Table 1 was used to replace Intermediate 1-7a in the synthesis method of Compound BH-1. For the synthesis of compounds BH-13 to BH-16, Intermediate 2 in Table 2 was used to replace Intermediate 1-7a in the synthesis method of Compound BH-1. For the synthesis of compound BH-17, Intermediate 1 in Table 2 was used to replace Intermediate 1-6 in the synthesis method of Compound BH-1, and Intermediate 2 in Table 2 was used to replace Intermediate 1-7a in the synthesis method of Compound BH-1.

The 1H NMR and mass spectra of the prepared compounds BH-8 to BH-17 are as follows:

The 1H NMR (400 MHz, DMSO) characterization of compound BH-8 is: δ 9.01 (s, 1H), 8.24 (s, 1H), 8.06 (s, 1H), 7.94 (s, 1H), 7.86 (s, 1H), 7.74 (d, J=8.1 Hz, 3H), 7.61 (s, 1H), 7.55-7.27 (m, 9H), 7.09 (s, 4H), 4.90 (d, J=2.2 Hz, 2H). The mass spectrum (MS) characterization of compound BH-8 is m/z: 640.30.

The 1H NMR (400 MHz, DMSO) characterization of compound BH-9 is: δ 9.01 (s, 1H), 8.24 (s, 1H), 8.06 (s, 1H), 7.94 (s, 1H), 7.86 (s, 1H), 7.73 (s, 1H), 7.61 (s, 1H), 7.53-7.28 (m, 6H), 7.09 (s, 4H), 4.90 (d, J=2.0 Hz, 2H). The mass spectrum (MS) characterization of compound BH-9 is m/z: 645.33.

The 1H NMR (400 MHz, DMSO) characterization of compound BH-10 is: δ 8.25 (s, 1H), 7.98 (s, 1H), 7.93-7.67 (m, 4H), 7.58 (d, J=28.3 Hz, 2H), 7.48-7.22 (m, 6H), 7.09 (s, 4H), 4.83 (d, J=5.5 Hz, 2H). The mass spectrum (MS) characterization of compound BH-10 is m/z: 684.32.

The 1H NMR (400 MHz, DMSO) characterization of compound BH-11 is: δ 7.85 (s, 1H), 7.73 (s, 1H), 7.61 (s, 1H), 7.40 (d, J=4.2 Hz, 4H), 7.09 (s, 4H), 4.83 (d, J=4.2 Hz, 2H). The mass spectrum (MS) characterization of compound BH-11 is m/z: 611.31.

The 1H NMR (400 MHz, DMSO) characterization of compound BH-12 is: δ 7.85 (s, 1H), 7.73 (s, 1H), 7.62 (s, 1H), 7.40 (d, J=3.8 Hz, 4H), 7.09 (s, 4H), 4.83 (d, J=3.4 Hz, 2H). The mass spectrum (MS) characterization of compound BH-12 is m/z: 663.34.

The 1H NMR (400 MHz, DMSO) characterization of compound BH-13 is: δ 9.01 (s, 1H), 8.23 (d, J=12.9 Hz, 5H), 8.06 (s, 1H), 7.94 (s, 1H), 7.86 (s, 1H), 7.73 (s, 1H), 7.61 (s, 1H), 7.53-7.27 (m, 10H), 7.09 (s, 4H), 4.90 (d, J=2.2 Hz, 2H). The mass spectrum (MS) characterization of compound BH-13 is m/z: 637.28.

The 1H NMR (400 MHz, DMSO) characterization of compound BH-14 is: δ 8.23 (d, J=15.1 Hz, 5H), 7.98 (s, 1H), 7.93-7.68 (m, 4H), 7.58 (d, J=28.3 Hz, 2H), 7.47-7.23 (m, 10H), 7.09 (s, 4H), 4.83 (d, J=5.5 Hz, 2H). The mass spectrum (MS) characterization of compound BH-14 is m/z: 676.27.

The 1H NMR (400 MHz, DMSO) characterization of compound BH-15 is: δ 8.21 (s, 4H), 7.85 (s, 1H), 7.73 (s, 1H), 7.61 (s, 1H), 7.49-7.28 (m, 8H), 7.09 (s, 4H), 4.83 (d, J=4.2 Hz, 2H). The mass spectrum (MS) characterization of compound BH-15 is m/z: 603.26.

The 1H NMR (400 MHz, DMSO) characterization of compound BH-16 is: δ 8.21 (s, 4H), 7.91 (s, 1H), 7.78 (s, 1H), 7.64 (s, 1H), 7.40 (t, J=4.6 Hz, 8H), 7.09 (s, 4H), 4.87 (d, J=4.2 Hz, 2H). The mass spectrum (MS) characterization of compound BH-16 is m/z: 655.29.

The 1H NMR (400 MHz, DMSO) characterization of compound BH-17 is: δ 4.85 (d, J=0.5 Hz, 2H). The mass spectrum (MS) characterization of compound BH-17 is m/z: 674.41.

### Physical Characterization:

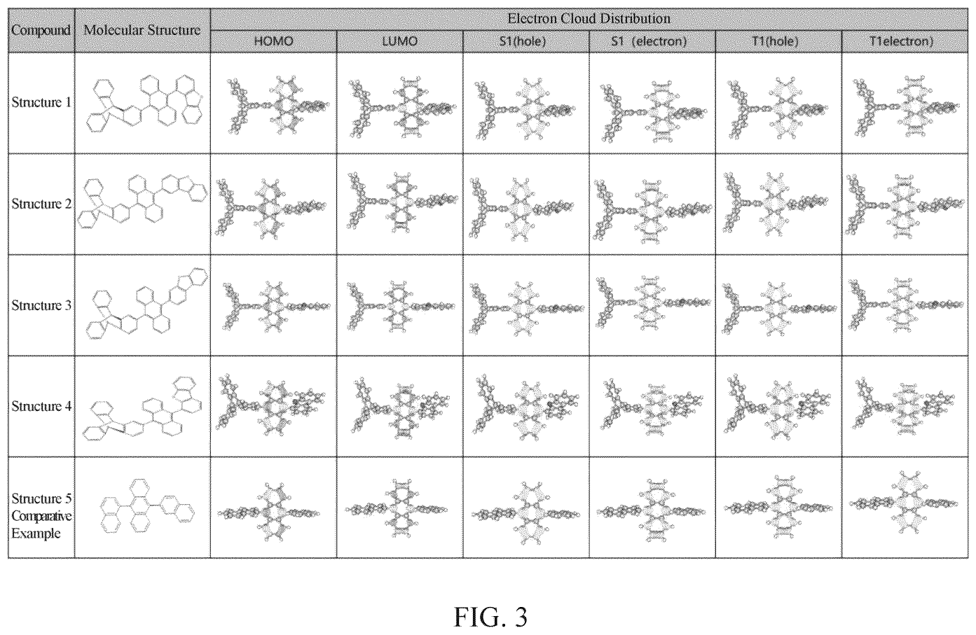

Molecular simulation software was employed to simulate the electron cloud distribution of the compounds, with results shown in FIG. 3. The simulation results demonstrate that the HOMO (Highest Occupied Molecular Orbital)/LUMO (Lowest Unoccupied Molecular Orbital)/S1 (First Singlet Excited State)/T1 (First Triplet Excited State) electron clouds of the compounds of the present disclosure are all distributed on the anthracene moiety. Blue light host materials require wide bandgap characteristics, which anthracene satisfies perfectly (exhibiting a large energy gap between HOMO and LUMO levels). This material characteristic enables better matching with blue light emission.

When using the compounds provided in the disclosure as blue light host materials, the T1 electron clouds of the host material are entirely localized on the anthracene unit. As anthracene possesses a fused-ring structure with excellent conjugation, this imparts a relatively low T1 level to the blue light host material. Consequently, when employing these compounds as host materials, adjacent functional layer materials can be selected from a wider range of options (requiring only that their T1 levels be higher than that of the host material). This effectively confines excitons within the emission layer, significantly improving exciton utilization efficiency and thereby markedly enhancing device luminescence efficiency. Furthermore, when using the inventive compounds as blue light host materials, selecting blue light guest materials with T1 levels higher than that of the host material can facilitate more efficient TTF (Triplet-Triplet Fusion) effects on the host material, leading to further improvements in device efficiency.

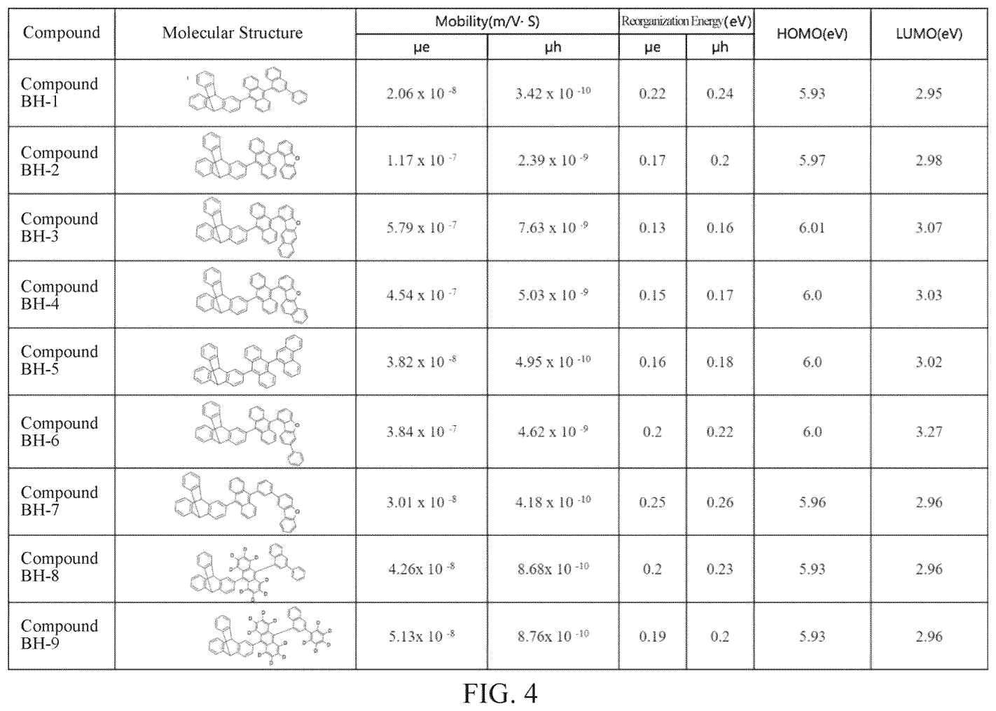

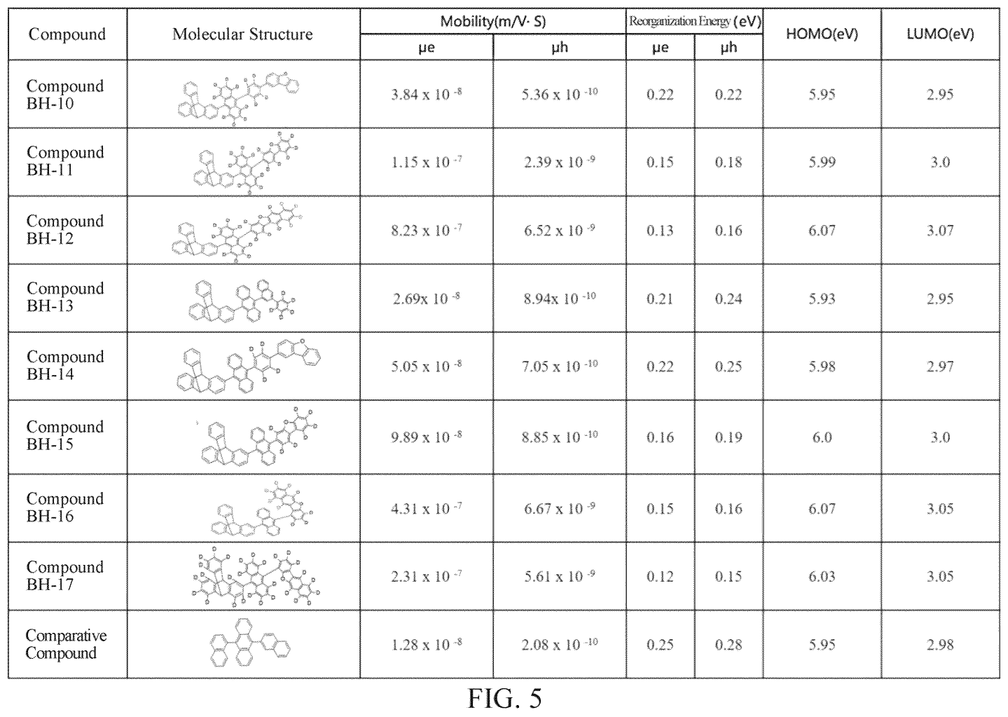

The present disclosure also measured certain performance parameters of some compounds. The HOMO/LUMO energy levels were determined using AC3, CV, and UV spectroscopy. The mobility was tested using the SCLC method (HOD: ITO/P-HTL(4%) 10 nm/BH 200 nm/Ag 80 nm; EOD: ITO/HBL 10 nm/BH 150 nm/ETL:Liq(5:5) 5 nm/Mg:Ag(8:2) 80 nm), while the reorganization energy was obtained through simulation calculations. The test results are shown in FIGS. 4 and 5.

Here, HOD refers to a device with only a hole-transport region, and EOD refers to a device with only an electron-transport region. BH represents the blue light host material to be tested, and P-HTL is a composite material of HAT-CN and NPB. The structures of the materials used are as follows:

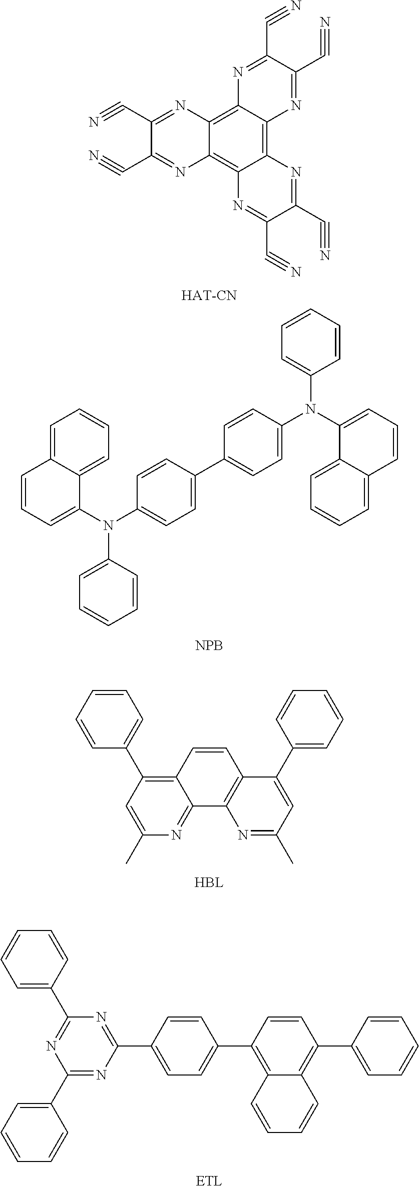

As shown in FIGS. 4 and 5, compared with the comparative compounds, the compounds of the present disclosure containing the group represented by Chemical Formula 1 exhibit no significant changes in HOMO/LUMO energy levels. This is because the electron clouds of both HOMO and LUMO are primarily localized on the anthracene ring. All tested compounds incorporate a triptycene group, whose rigid carbon framework provides exceptional stability, resulting in a more stable molecular geometry that resists deformation under applied electric fields and demonstrates lower reorganization energy. Relative to the comparative compounds, the inventive compounds exhibit higher electron mobility.

Furthermore, the introduction of bulky triptycene steric groups in these blue light host materials can effectively reduce intermolecular interactions. This prevents spectral red-shift of the host materials in thin-film states, which could otherwise lead to inefficient exciton transfer from host to guest materials. Such inefficient energy transfer would reduce the luminescence efficiency of guest materials and consequently degrade device performance. The triptycene modification successfully addresses this issue.

Additionally, these blue light host materials also incorporate a dibenzo structure (containing heteroatoms) in the molecule. The dibenzo structure has good conjugation effect. When the dibenzo structure is directly connected to anthracene, it can increase the conjugation of the material, giving the material deeper and faster mobility, enabling better electron transport, thereby improving device efficiency. At the same time, the molecule contains heteroatoms (0, S, N), and the presence of heteroatoms gives the material greater polarity, which will improve the interface energy level between the material and adjacent functional layers, enhance the injection characteristics of the material, further strengthen the interaction with adjacent functional layers, and reduce the operating voltage of the device.

### Glass Transition Temperature Tg

The glass transition temperature (Tg) determines the thermal stability of the material during evaporation deposition. A higher Tg indicates better thermal stability of the material.

The measuring instrument was a DSC differential scanning calorimeter. The test atmosphere was nitrogen, with a heating rate of 10° C./min and a temperature range of 50-380° C. The measured Tg values of each compound are listed in Table 1:

TABLE 1

| Compound | Tg(° C.) |
|---|---|
| Compound BH-1 | 130 |
| Compound BH-2 | 147 |
| Compound BH-3 | 142 |
| Compound BH-4 | 151 |
| Compound BH-5 | 135 |
| Compound BH-6 | 129 |
| Compound BH-7 | 132 |
| Compound BH-8 | 130 |
| Compound BH-9 | 129 |
| Compound BH-10 | 137 |
| Compound BH-11 | 139 |
| Compound BH-12 | 143 |
| Compound BH-13 | 132 |
| Compound BH-14 | 136 |
| Compound BH-15 | 145 |
| Compound BH-16 | 142 |
| Compound BH-17 | 145 |
| Comparative Compound | 124 |

In the blue light host materials of this embodiment, the triptycene group serves as a rigid three-dimensional structure. This rigid configuration effectively enhances the material's Tg (glass transition temperature). A higher Tg improves the thermodynamic stability of the material, preventing decomposition during evaporation deposition processes while maintaining excellent film-forming properties. These characteristics constitute fundamental requirements for enabling successful vacuum deposition and ensuring extended operational lifetime.

Device Fabrication and Testing Fabricate organic electroluminescent devices with the following structure: the anode of the device was ITO with a thickness of 10 nm; the material of the hole injection layer was HAT-CN with a thickness of 10 nm; the material of the hole transport layer was NPB with a thickness of 100 nm; the material of the electron blocking layer was mCBP with a thickness of 35 nm; the material of the light-emitting layer was a mixture of blue host material and BD (3 wt %) with a thickness of 20 nm; wherein, the compounds used as blue host material were different in different embodiments; the material of the hole blocking layer was HB with a thickness of 5 nm; the material of the electron transport layer was a mixture of ET and LiQ (1:1) with a thickness of 30 nm; the material of the electron injection layer was Yb with a thickness of 1 nm; the material of the cathode was Mg/Ag(1:9) with a thickness of 100 nm. The blue host materials corresponding to different embodiments are shown in Table 2.

The fabrication process of the organic electroluminescent devices in the embodiments of the present disclosure was as follows:
(1) A glass plate with ITO was ultrasonically treated in a cleaning agent, rinsed in deionized water, degreased by ultrasonication in an acetone-ethanol mixed solvent, and baked in a clean environment until all moisture was completely removed;
(2) The glass substrate with the anode was placed in a vacuum chamber, and the vacuum was pumped to 1×10⁻⁵˜1×10⁻⁶ Pa. A hole injection material was vacuum-deposited on the anode layer film to form a hole injection layer;
(3) A hole transport material was vacuum-deposited on the hole injection layer to form a hole transport layer;
(4) An electron blocking layer of the device was vacuum-deposited on the hole transport layer;
(5) A light-emitting layer of the device was vacuum-deposited on the electron blocking layer. The light-emitting layer included a blue light host material and a dopant material, using a multi-source co-evaporation method;
(6) A hole blocking layer of the device was vacuum-deposited on the light-emitting layer;
(7) An electron transport layer of the device was vacuum-deposited on the hole blocking layer;
(8) Yb was vacuum-deposited on the electron transport layer (ETL) as an electron injection layer;
(9) An Mg/Ag (1:9) layer was vacuum-deposited on the electron injection layer as the cathode of the device.

The materials used in the aforementioned fabrication process were as follows:

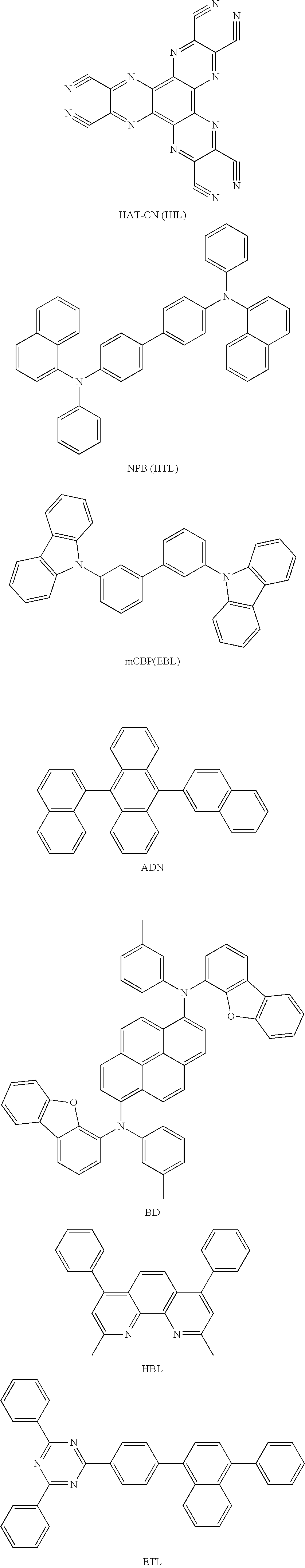

As shown in Table 2, the blue host material in the organic electroluminescent device prepared in Example 1 was compound BH-1; the blue host material in the organic electroluminescent device prepared in Example 2 was compound BH-2; similarly, the blue host material in the organic electroluminescent device prepared in Example 17 was compound BH-17; the blue host material in the organic electroluminescent device prepared in the Comparative Example was the comparative compound (ADN).

The organic electroluminescent devices prepared in the above examples were tested for driving voltage and luminous efficiency at a fixed current density of 15 mA/cm². The test results are shown in Table 2.

TABLE 2

| | Blue Host | | Emission | Effi- | Life- |
|---|---|---|---|---|---|
| Example | Material | Voltage | Peak (nm) | ciency | time |
| Example 1 | Compound BH-1 | 99% | 460 | 108% | 119% |
| Example 2 | Compound BH-2 | 95% | 460 | 122% | 116% |
| Example 3 | Compound BH-3 | 94% | 459 | 126% | 113% |
| Example 4 | Compound BH-4 | 95% | 461 | 123% | 106% |
| Example 5 | Compound BH-5 | 99% | 460 | 123% | 108% |
| Example 6 | Compound BH-6 | 96% | 459 | 127% | 116% |
| Example 7 | Compound BH-7 | 98% | 460 | 125% | 132% |
| Example 8 | Compound BH-8 | 100% | 461 | 126% | 129% |
| Example 9 | Compound BH-9 | 98% | 461 | 128% | 135% |
| Example 10 | Compound BH-10 | 97% | 461 | 127% | 123% |
| Example 11 | Compound BH-11 | 96% | 462 | 128% | 117% |
| Example 12 | Compound BH-12 | 94% | 461 | 131% | 122% |
| Example 13 | Compound BH-13 | 94% | 459 | 116% | 130% |
| Example 14 | Compound BH-14 | 98% | 460 | 122% | 128% |
| Example 15 | Compound BH-15 | 94% | 461 | 128% | 115% |
| Example 16 | Compound BH-16 | 94% | 459 | 124% | 118% |
| Example 17 | Compound BH-17 | 93% | 458 | 132% | 125% |
| Comparative | Comparative | 100% | 460 | 100% | 100% |
| Example 1 | Compound | | | | |

In Table 2, all test data are normalized values, with the results of the Comparative Example set as 100%. As shown in Table 2, although the comparative compound also contains an anthracene group, when the host material in the organic layer of the electroluminescent device is replaced with the compounds provided in the disclosure as the blue host material, the device exhibits reduced driving voltage, enhanced efficiency, and prolonged lifetime compared to the comparative compound. This demonstrates that by introducing triptycene groups and Q groups, the compounds disclosed herein can improve the lifetime and efficiency of organic electroluminescent devices while reducing their driving voltage.

What is claimed is:

1. A compound represented by Chemical Formula 1: 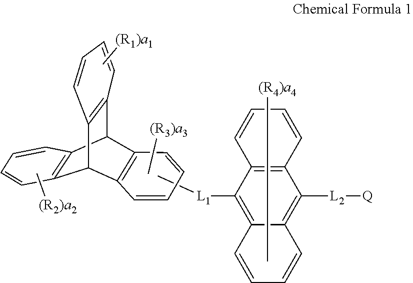
wherein, L₁ and L₂ are each independently selected from a single bond or a linking group, the linking group being selected from divalent arylene or divalent heteroarylene;
Q is a substituted or unsubstituted 2 to 5 ring-fused aryl group, or a substituted or unsubstituted 2 to 5 ring-fused heteroaryl group;
R₁ to R₃ are the same or different from each other and are each independently selected from a group consisting of deuterium, fluorine, chlorine, substituted or unsubstituted alkyl having 1 to 12 carbon atoms, substituted or unsubstituted haloalkyl having 1 to 12 carbon atoms, substituted or unsubstituted alkoxy having 1 to 12 carbon atoms, substituted or unsubstituted cycloalkyl having 3 to 10 carbon atoms, substituted or unsubstituted aryl having 6 to 60 carbon atoms, and substituted or unsubstituted heteroaryl having 3 to 60 carbon atoms;
R₄ is selected from deuterium, halogen, cyano, substituted or unsubstituted alkylamino having 1 to 20 carbon atoms, substituted or unsubstituted alkyl having 1 to 20 carbon atoms, substituted or unsubstituted alkoxy having 1 to 20 carbon atoms, substituted or unsubstituted aryloxy having 6 to 30 ring carbon atoms, substituted or unsubstituted arylthio having 6 to 30 ring carbon atoms, substituted or unsubstituted aryl having 6 to 30 ring carbon atoms, and substituted or unsubstituted heterocyclic group having 5 to 30 ring carbon atoms;
a₁ is an integer from 0 to 4; when a₁ is greater than 1, any two R₁ are the same or different;
a₂ is an integer from 0 to 4; when a₂ is greater than 1, any two R₂ are the same or different;
a₃ is an integer from 0 to 3; when a₃ is greater than 1, any two R₃ are the same or different;
a₄ is an integer from 0 to 8; when a₄ is greater than 1, any two R₄ are the same or different.

2. The compound according to claim 1, wherein Q is selected from a group consisting of the groups represented by Chemical Formulas 3-1 to 3-13: 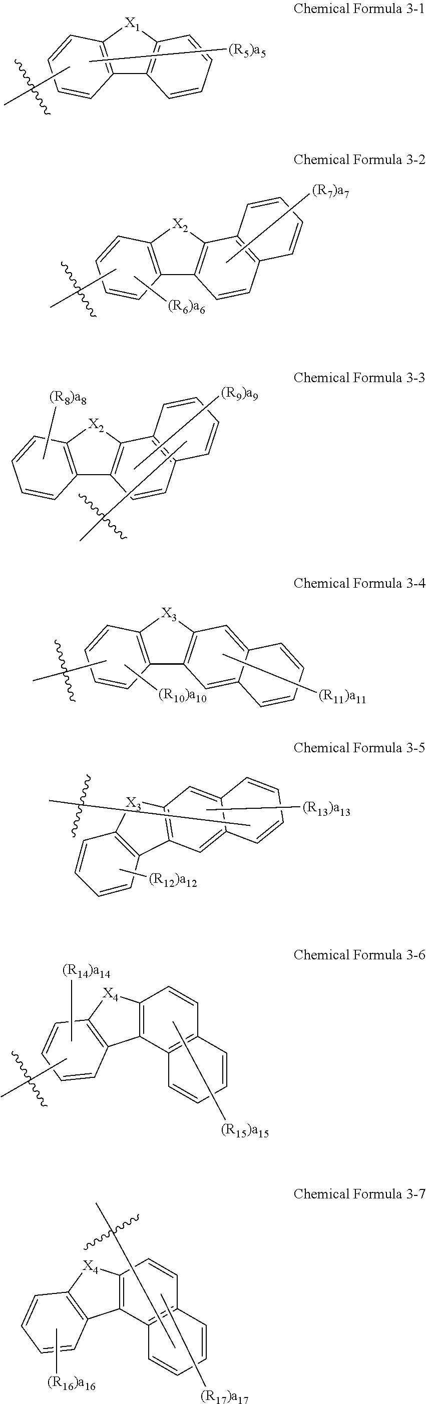 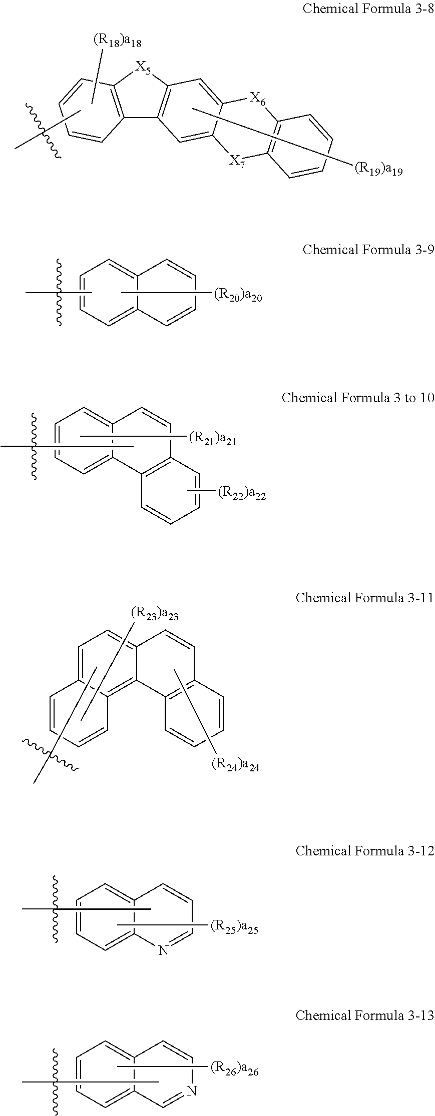
wherein, X₁ to X₅ are each independently selected from O, S, C(R)₂, N(R), or Si(R)₂;
X₆ to X₇ are each independently selected from a single bond, O, S, C(R)₂, N(R), or Si(R)₂, with the proviso that X₆ and X₇ are not simultaneously a single bond;
R is selected from the group consisting of hydrogen, deuterium, fluorine, chlorine, substituted or unsubstituted alkyl having 1 to 12 carbon atoms, substituted or unsubstituted haloalkyl having 1 to 12 carbon atoms, substituted or unsubstituted alkoxy having 1 to 12 carbon atoms, substituted or unsubstituted cycloalkyl having 3 to 10 carbon atoms, substituted or unsubstituted aryl having 6 to 60 carbon atoms, and substituted or unsubstituted heteroaryl having 3 to 60 carbon atoms;
R₅ to R₂₆ are the same or different from each other and are each independently selected from the group consisting of deuterium, fluorine, chlorine, substituted or unsubstituted alkyl having 1 to 12 carbon atoms, substituted or unsubstituted haloalkyl having 1 to 12 carbon atoms, substituted or unsubstituted alkoxy having 1 to 12 carbon atoms, substituted or unsubstituted cycloalkyl having 3 to 10 carbon atoms, substituted or unsubstituted aryl having 6 to 60 carbon atoms, and substituted or unsubstituted heteroaryl having 3 to 60 carbon atoms;
a₅ is an integer from 0 to 7; when a₅ is greater than 1, any two R₅ may be the same or different;
a₆ is an integer from 0 to 3; when a₆ is greater than 1, any two R₆ may be the same or different;
a₇ is an integer from 0 to 6; when a₇ is greater than 1, any two R₇ may be the same or different;
a₈ is an integer from 0 to 4; when a₈ is greater than 1, any two R₈ may be the same or different;
a₉ is an integer from 0 to 5; when a₉ is greater than 1, any two R₉ may be the same or different;
a₁₀ is an integer from 0 to 3; when a₁₀ is greater than 1, any two R₁₀ may be the same or different;
a₁₁ is an integer from 0 to 6; when a₁₁ is greater than 1, any two R₁₁ may be the same or different;
a₁₂ is an integer from 0 to 4; when a₁₂ is greater than 1, any two R₁₂ may be the same or different;
a₁₃ is an integer from 0 to 5; when a₁₃ is greater than 1, any two R₁₃ may be the same or different;
a₁₄ is an integer from 0 to 3; when a₁₄ is greater than 1, any two R₁₄ may be the same or different;
a₁₅ is an integer from 0 to 6; when a₁₅ is greater than 1, any two R₁₅ may be the same or different;
a₁₆ is an integer from 0 to 4; when a₁₆ is greater than 1, any two R₁₆ may be the same or different;
a₁₇ is an integer from 0 to 5; when a₁₇ is greater than 1, any two R₁₇ may be the same or different;
a₁₈ is an integer from 0 to 3; when a₁₈ is greater than 1, any two R₁₈ may be the same or different;
a₁₉ is an integer from 0 to 6; when a₁₉ is greater than 1, any two R₁₉ may be the same or different;
a₂₀ is an integer from 0 to 7; when a₂₀ is greater than 1, any two R₂₀ may be the same or different;
a₂₁ is an integer from 0 to 5; when a₂₁ is greater than 1, any two R₂₁ may be the same or different;
a₂₂ is an integer from 0 to 4; when a₂₂ is greater than 1, any two R₂₂ may be the same or different;
a₂₃ is an integer from 0 to 5; when a₂₃ is greater than 1, any two R₂₃ may be the same or different;
a₂₄ is an integer from 0 to 6; when a₂₄ is greater than 1, any two R₂₄ may be the same or different;
a₂₅ is an integer from 0 to 6; when a₂₅ is greater than 1, any two R₂₅ may be the same or different;
a₂₆ is an integer from 0 to 6; when a₂₆ is greater than 1, any two R₂₆ may be the same or different.

3. The compound according to claim 2, wherein Q is selected from the group consisting of a group represented by Chemical Formula 3-1, a group represented by Chemical Formula 3-2, a group represented by Chemical Formula 3-4, a group represented by Chemical Formula 3-6, a group represented by Chemical Formula 3-8a, a group represented by Chemical Formula 3-8b, a group represented by Chemical Formula 3-9, a group represented by Chemical Formula 3-10, a group represented by Chemical Formula 3-11a, a group represented by Chemical Formula 3-12a, and a group represented by Chemical Formula 3-13;

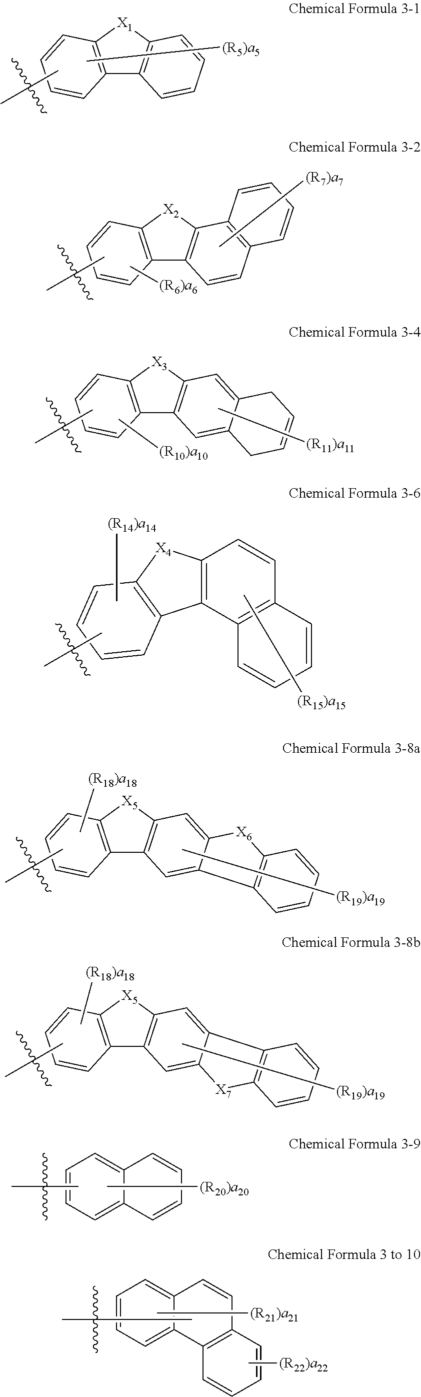

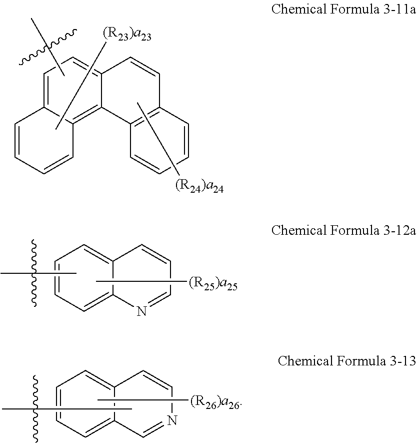

4. The compound according to claim 2, wherein X₁ to X₅ are each independently selected from O or S; X₆ to X₇ are each independently selected from a single bond, O, or S, with the proviso that X₆ and X₇ are not simultaneously a single bond.

5. The compound according to claim 3, wherein X₁ to X₅ are each independently selected from O or S; X₆ to X₇ are each independently selected from a single bond, O, or S, with the proviso that X₆ and X₇ are not simultaneously a single bond.

6. The compound according to claim 2, wherein R₅ to R₂₆ are each independently selected from the group consisting of deuterium, fully deuterated or non-deuterated methyl, and aryl groups, wherein the aryl groups are selected from fully deuterated or non-deuterated phenyl, fully deuterated or non-deuterated biphenyl, fully deuterated or non-deuterated naphthyl, fully deuterated or non-deuterated anthryl, fully deuterated or non-deuterated phenanthryl, and groups formed by connecting any two or three of the above via single bonds.

7. The compound according to claim 3, wherein R₅ to R₂₆ are each independently selected from the group consisting of deuterium, fully deuterated or non-deuterated methyl, and aryl groups, wherein the aryl groups are selected from fully deuterated or non-deuterated phenyl, fully deuterated or non-deuterated biphenyl, fully deuterated or non-deuterated naphthyl, fully deuterated or non-deuterated anthryl, fully deuterated or non-deuterated phenanthryl, and groups formed by connecting any two or three of the above via single bonds.

8. The compound according to claim 1, wherein L₁ and L₂ are each independently selected from a single bond, fully deuterated or non-deuterated phenylene, fully deuterated or non-deuterated biphenylene, fully deuterated or non-deuterated naphthylene, fully deuterated or non-deuterated anthrylene, fully deuterated or non-deuterated phenanthrylene, and groups formed by connecting any two or three of the above via single bonds.

9. The compound according to claim 1, wherein R₁ to R₄ are all deuterium; a₁ is 0 or 4; a₂ is 0 or 4; a₃ is 0 or 3; a₄ is 0 or 8; and
a₁ to a₃ are simultaneously 0, or simultaneously non-zero.

10. The compound according to claim 1, wherein the compound is selected from the group consisting of the following compounds:

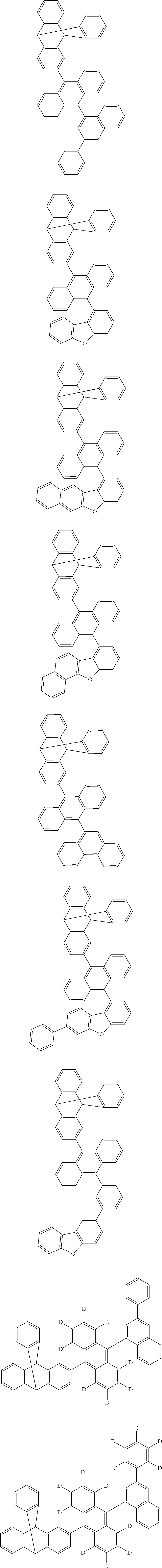

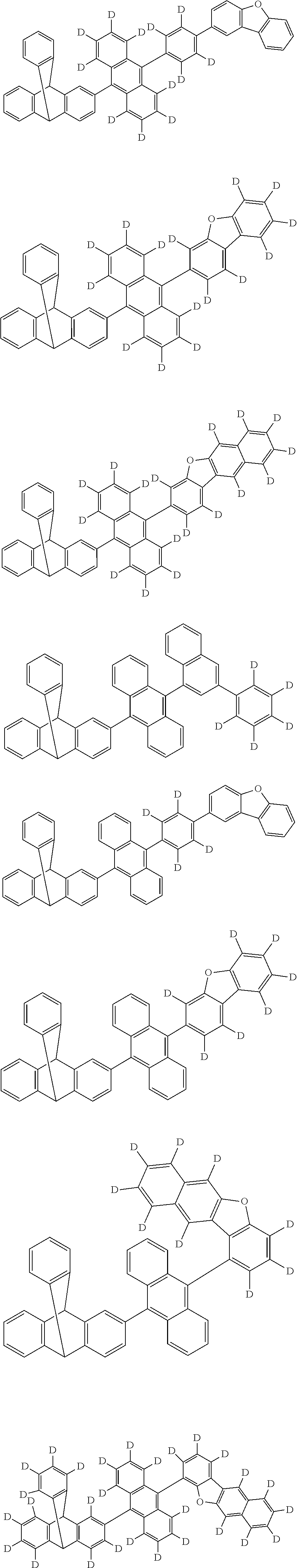

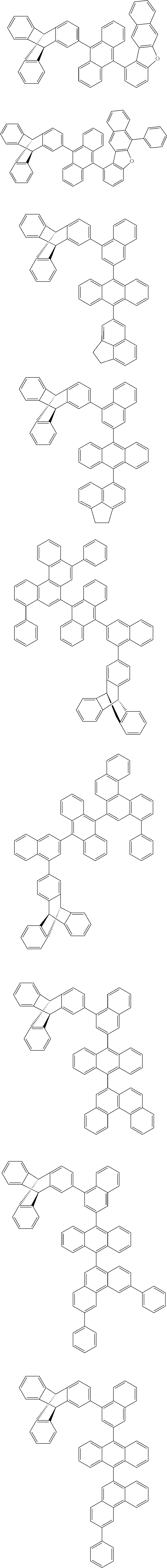

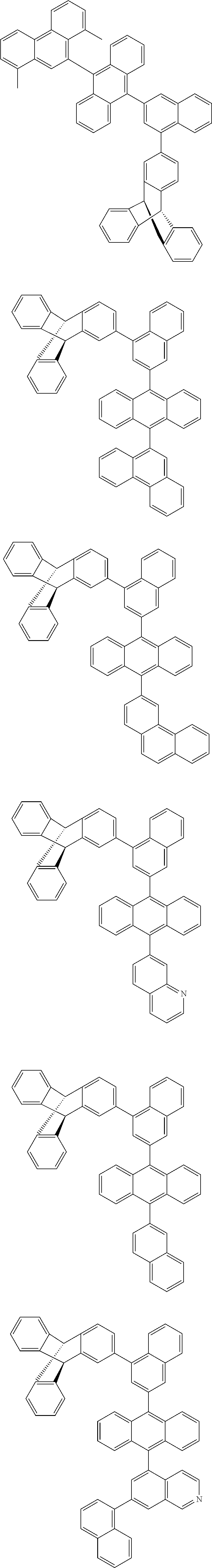

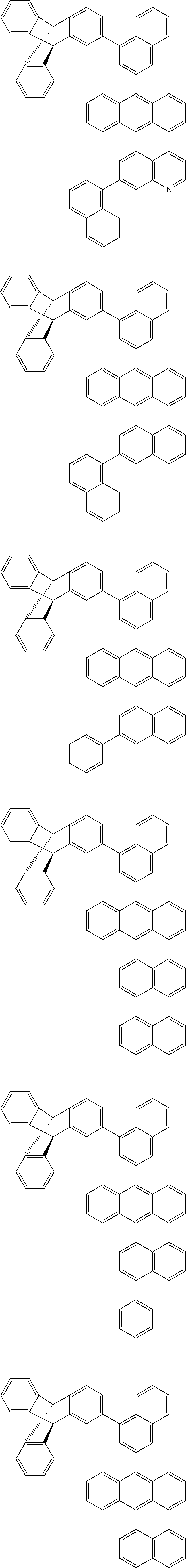

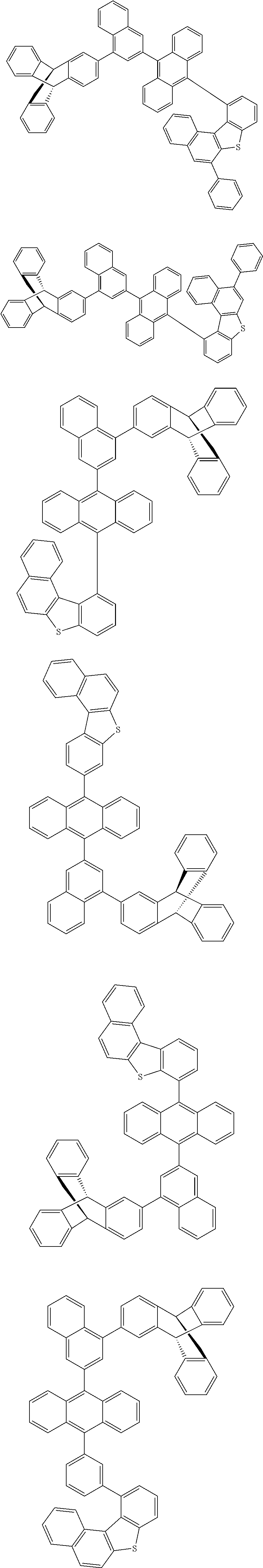

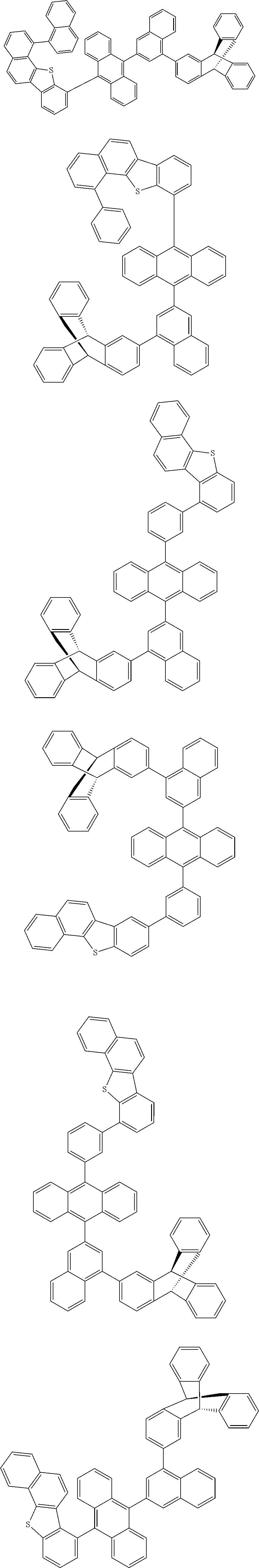

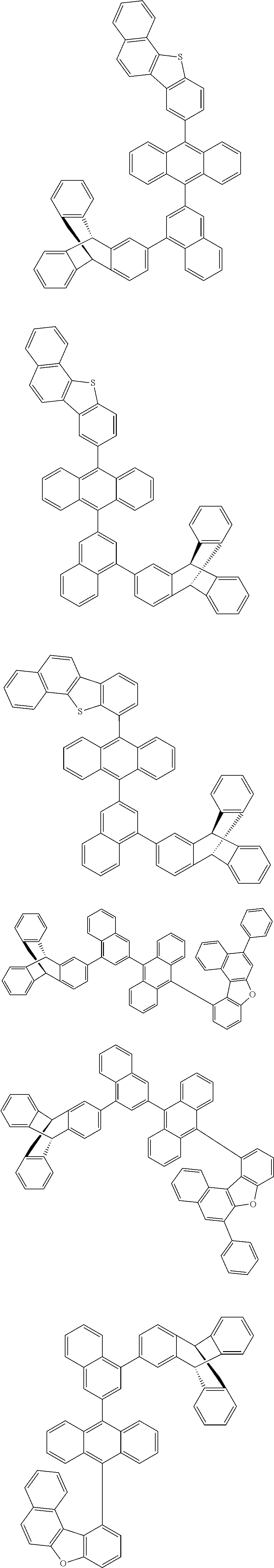

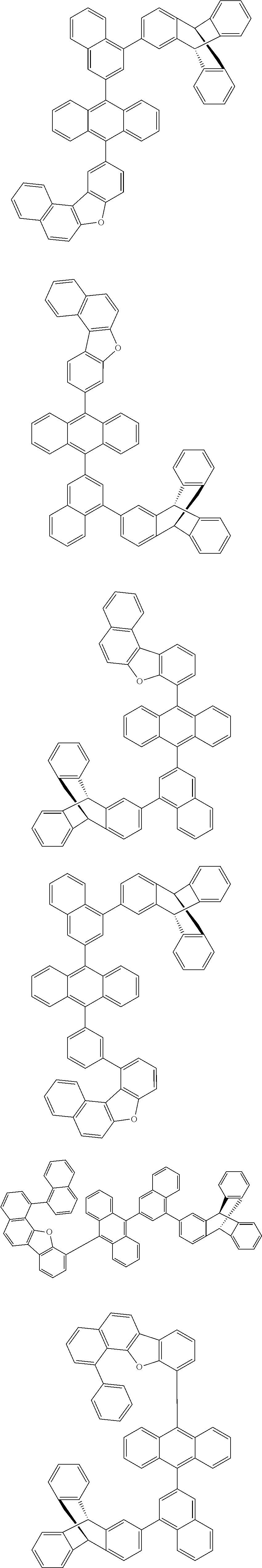

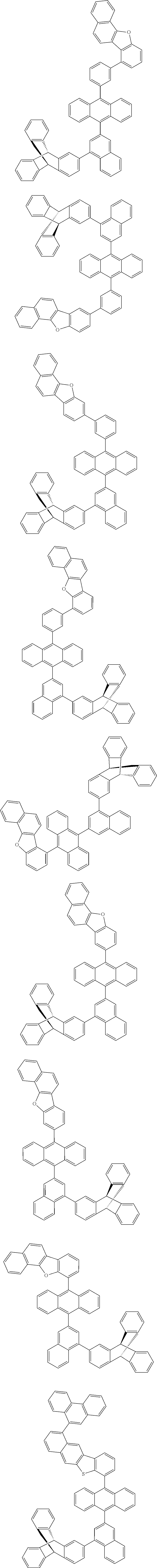

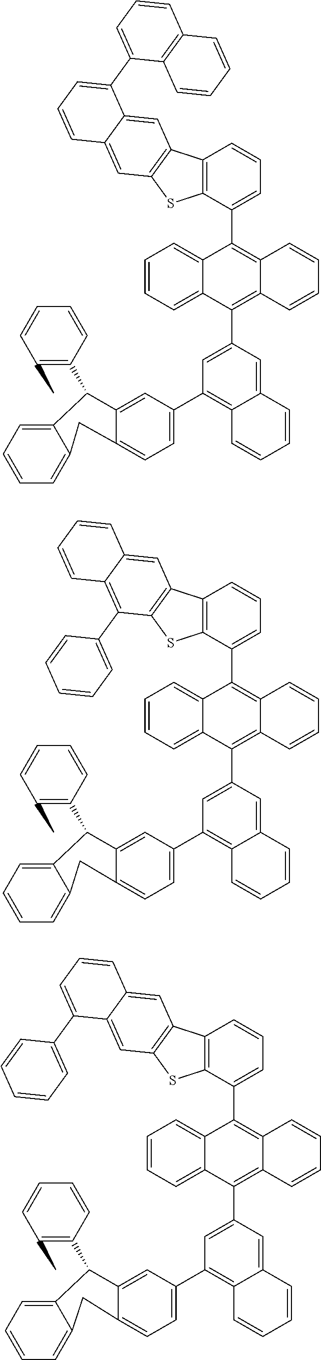

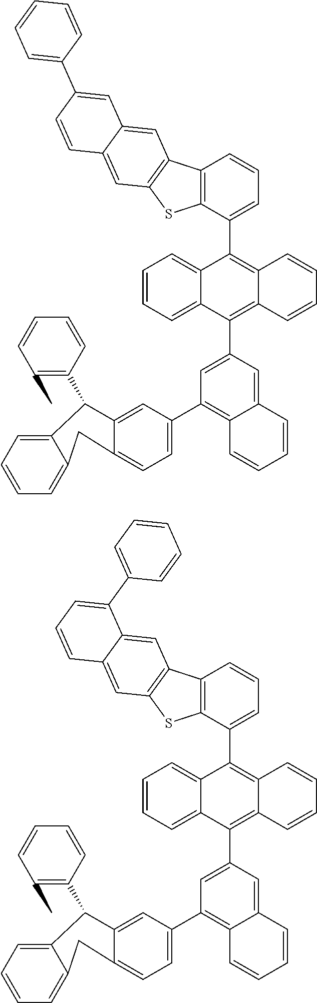

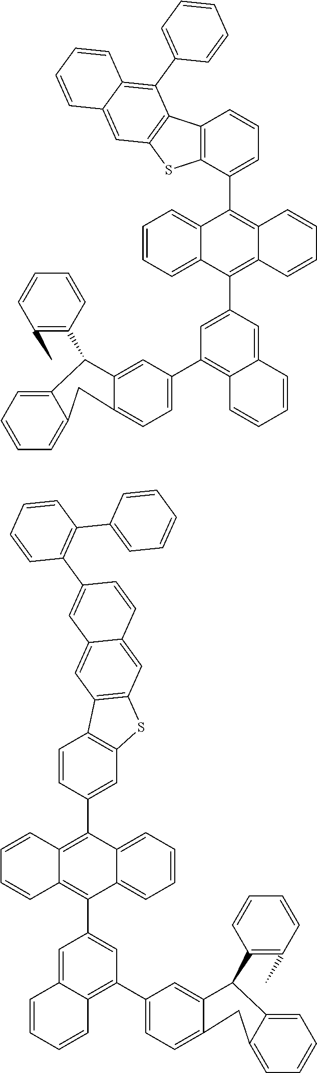

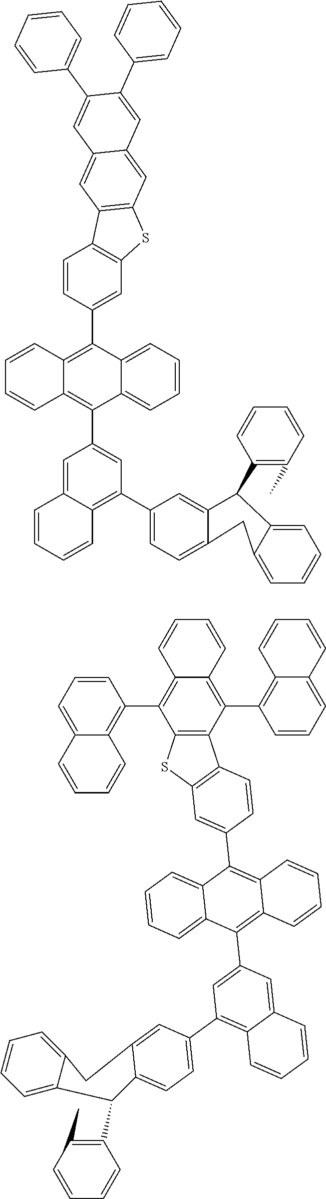

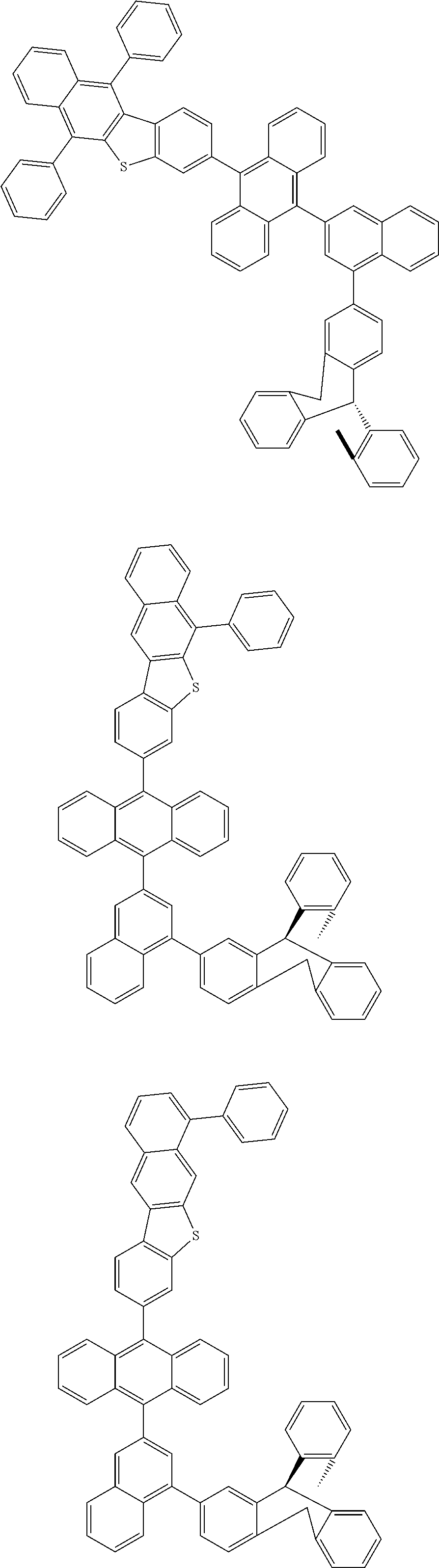

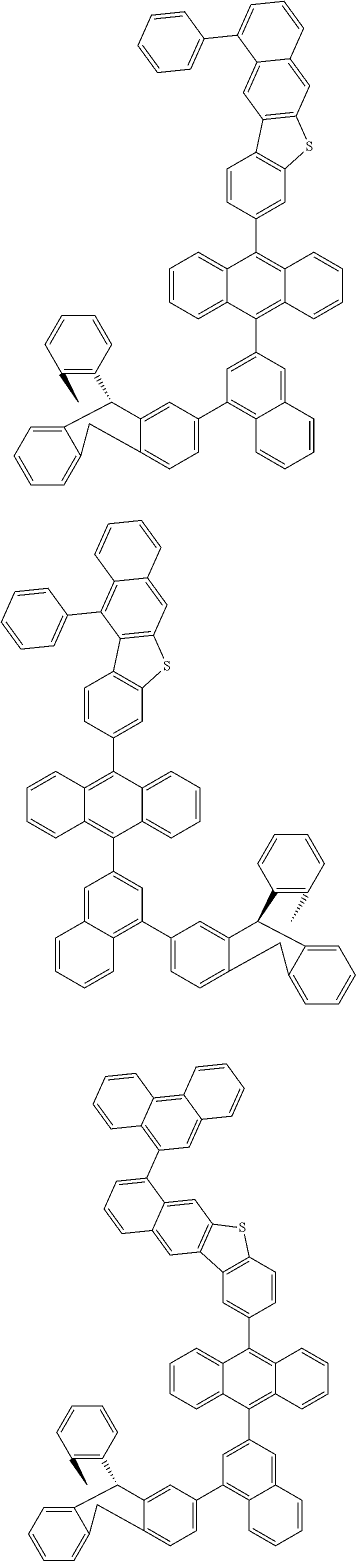

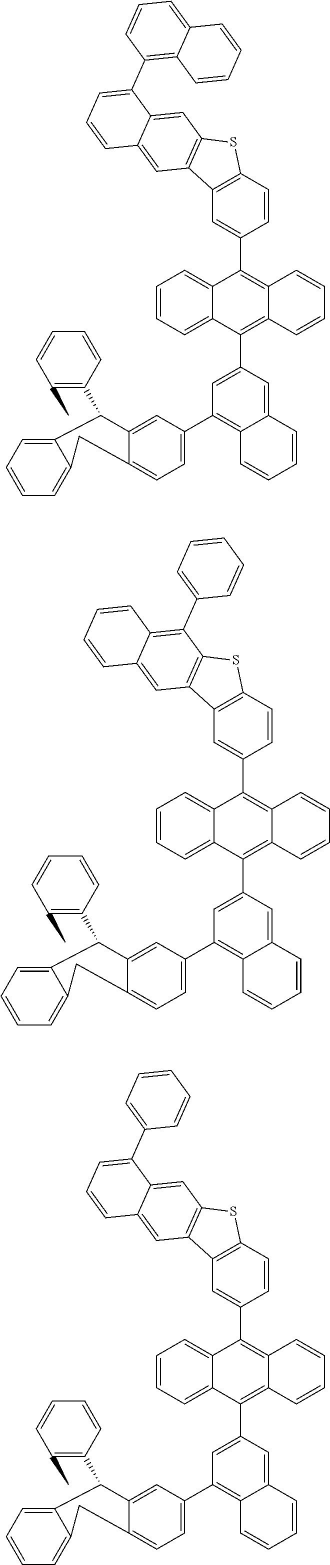

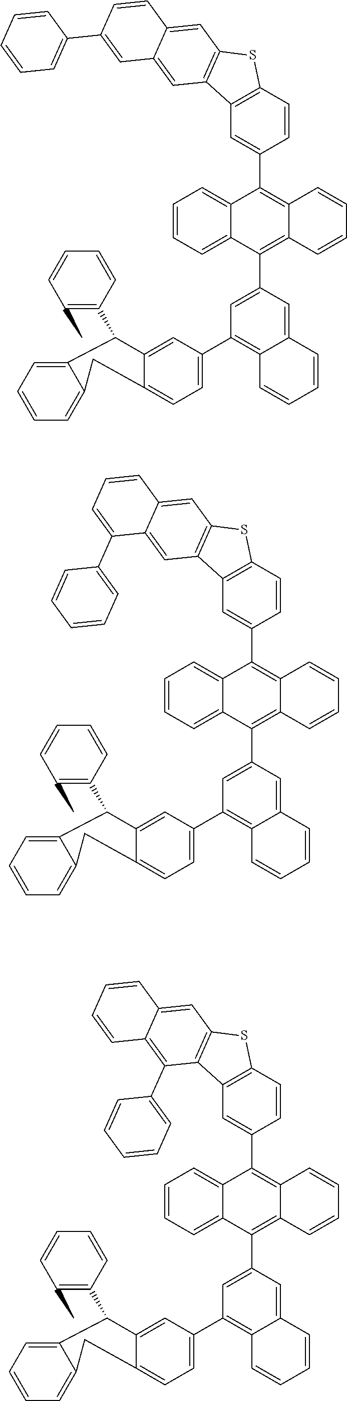

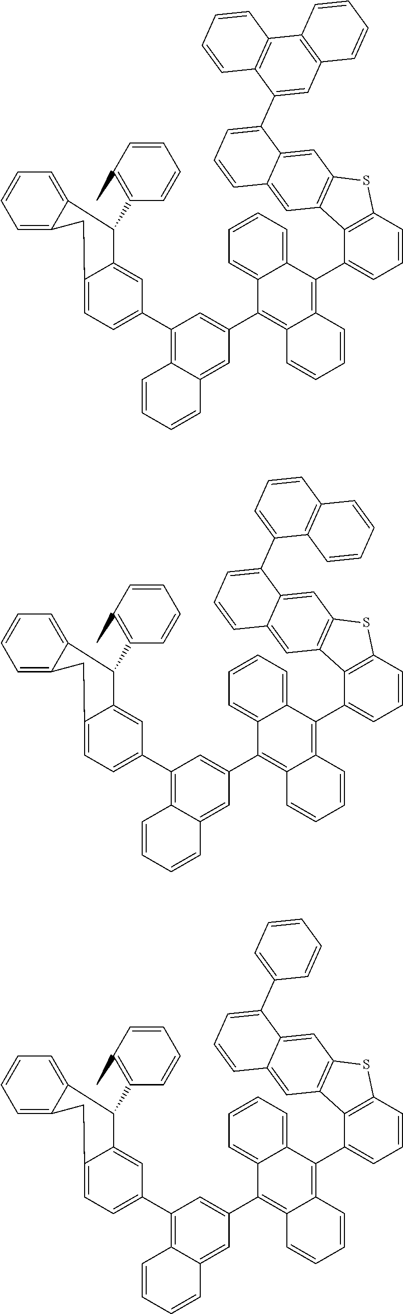

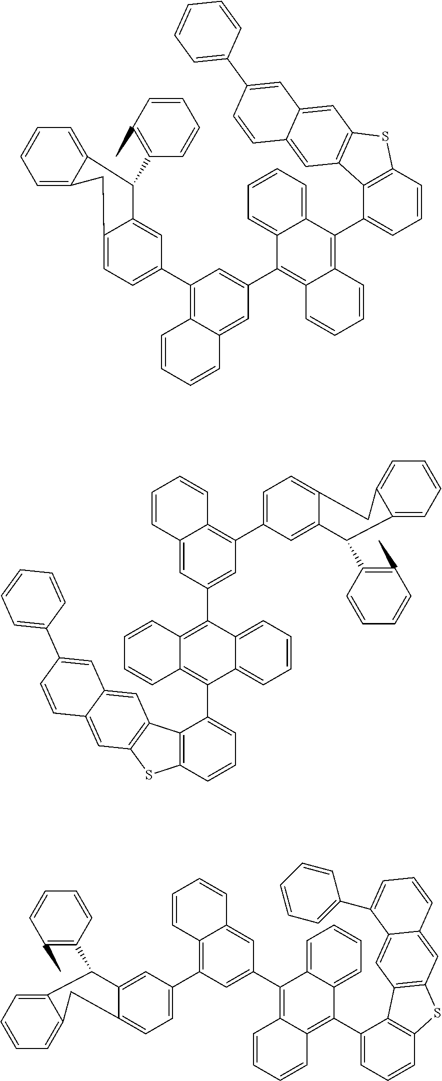

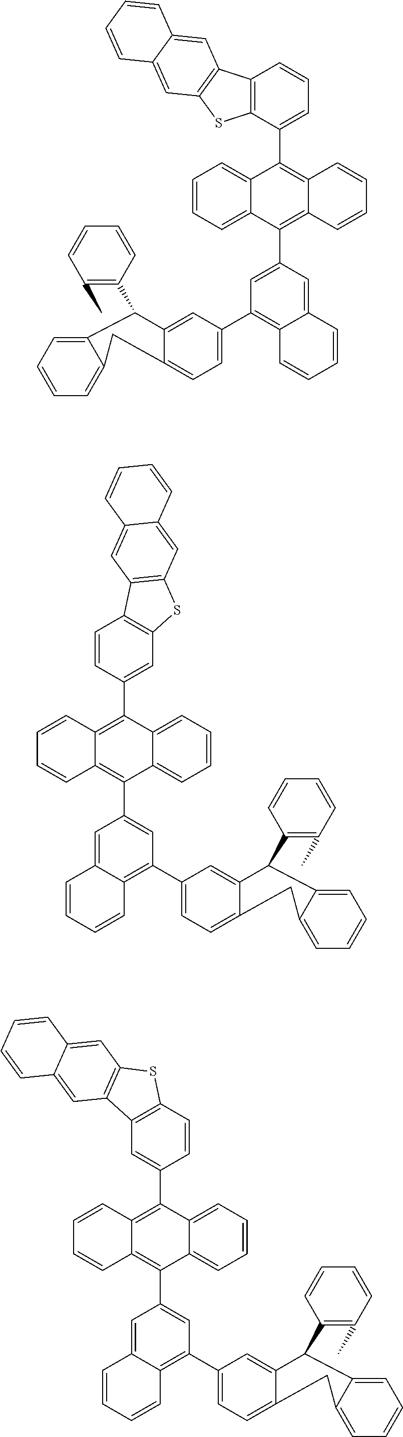

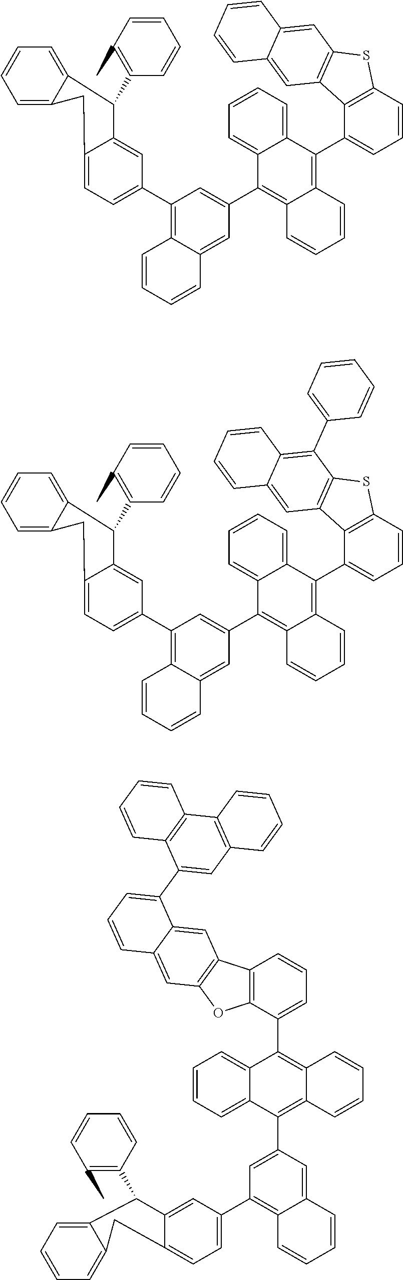

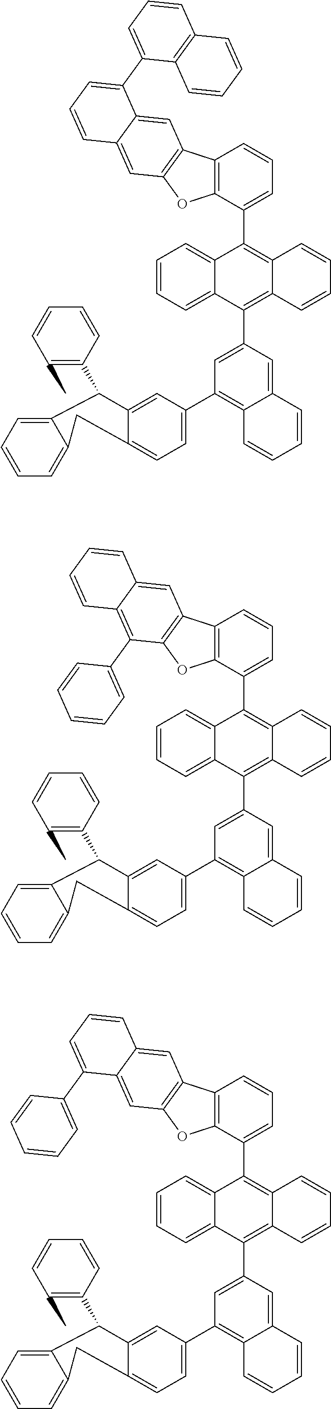

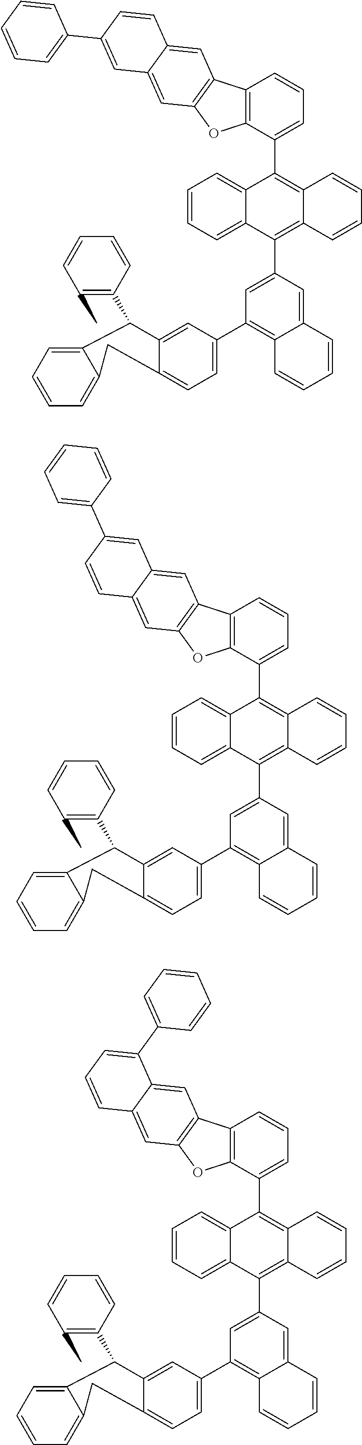

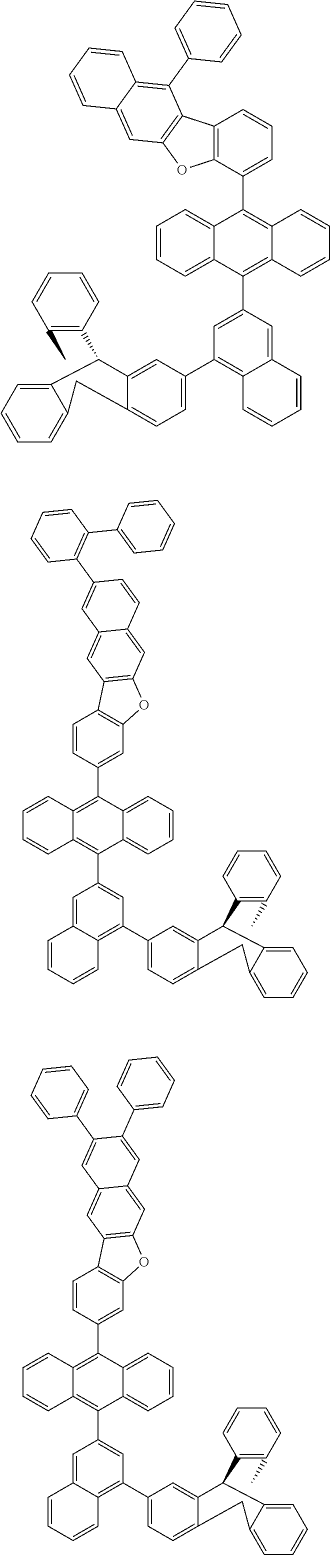

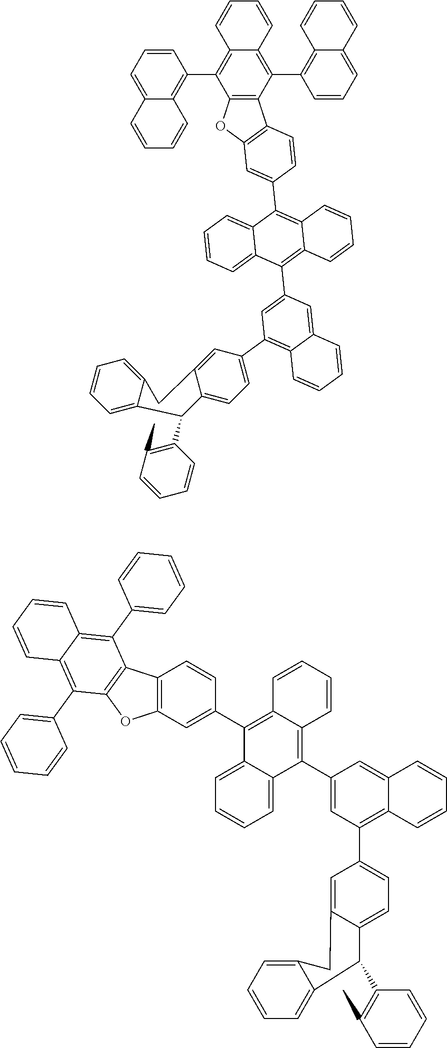

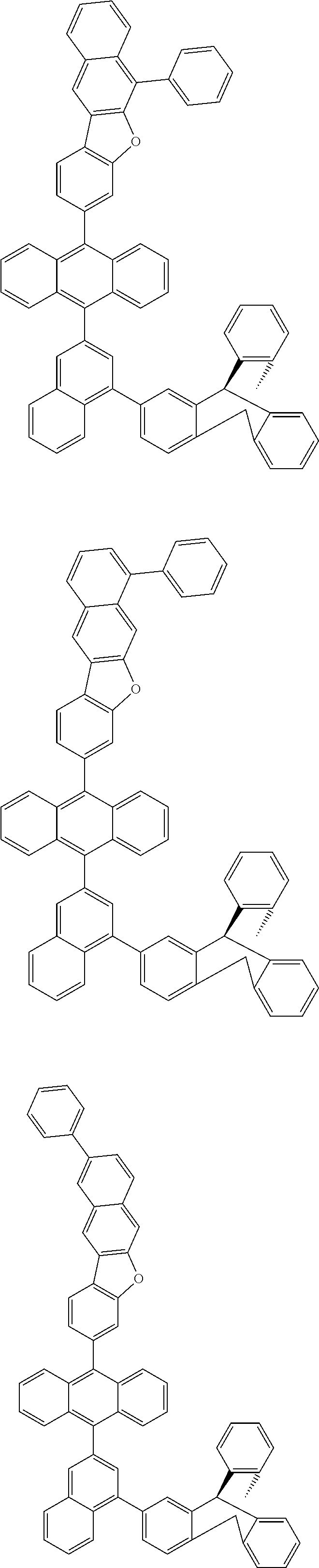

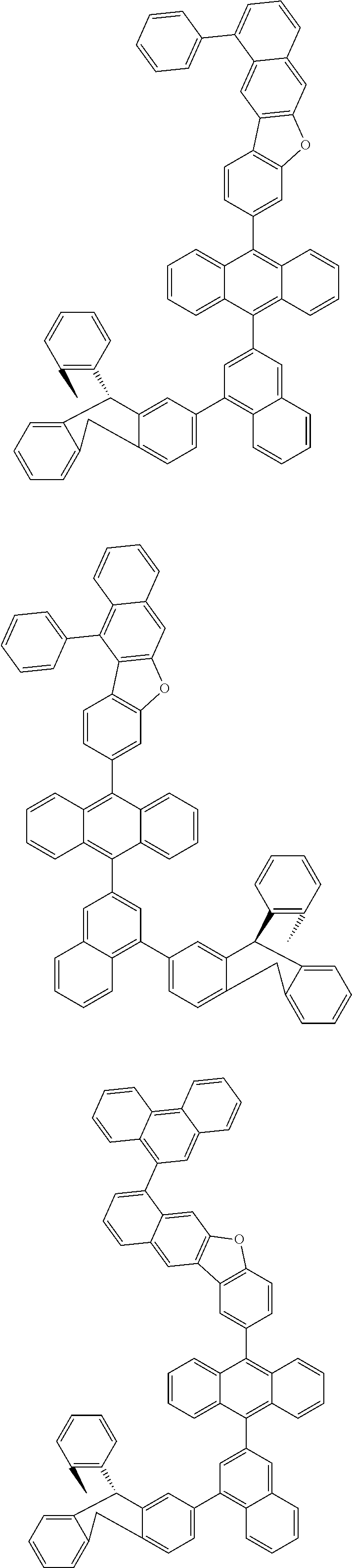

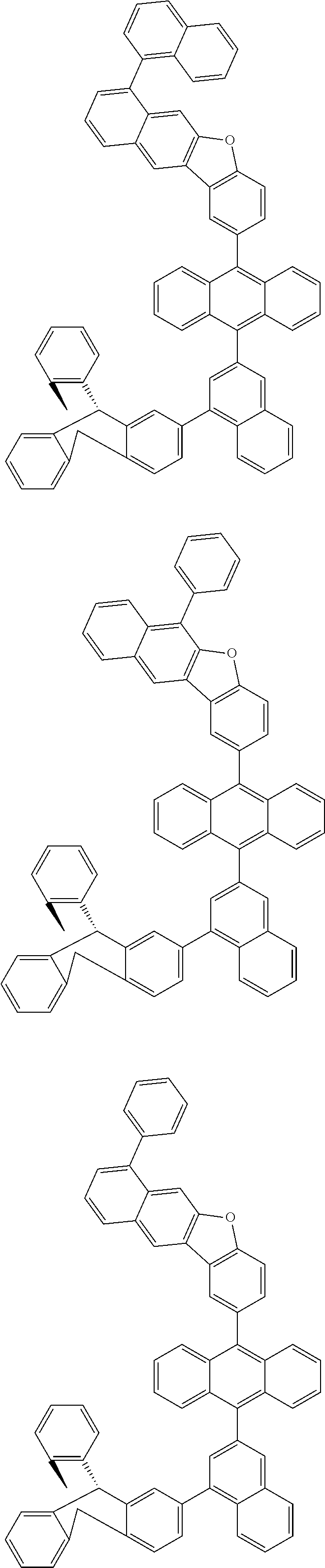

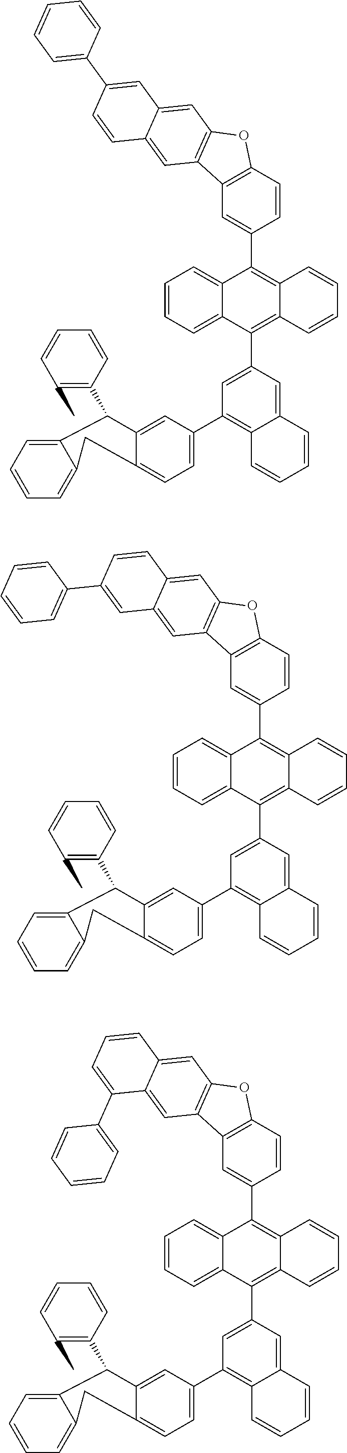

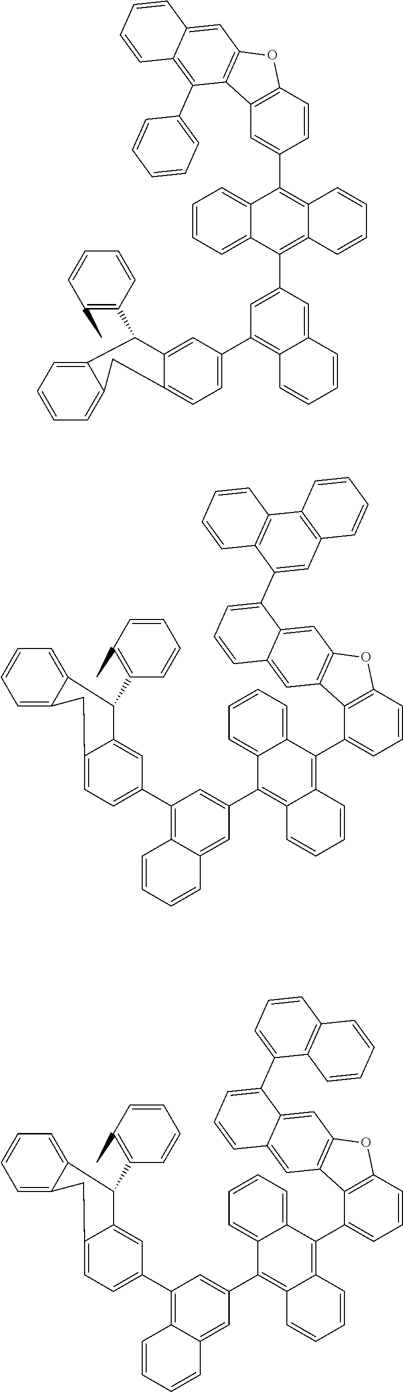

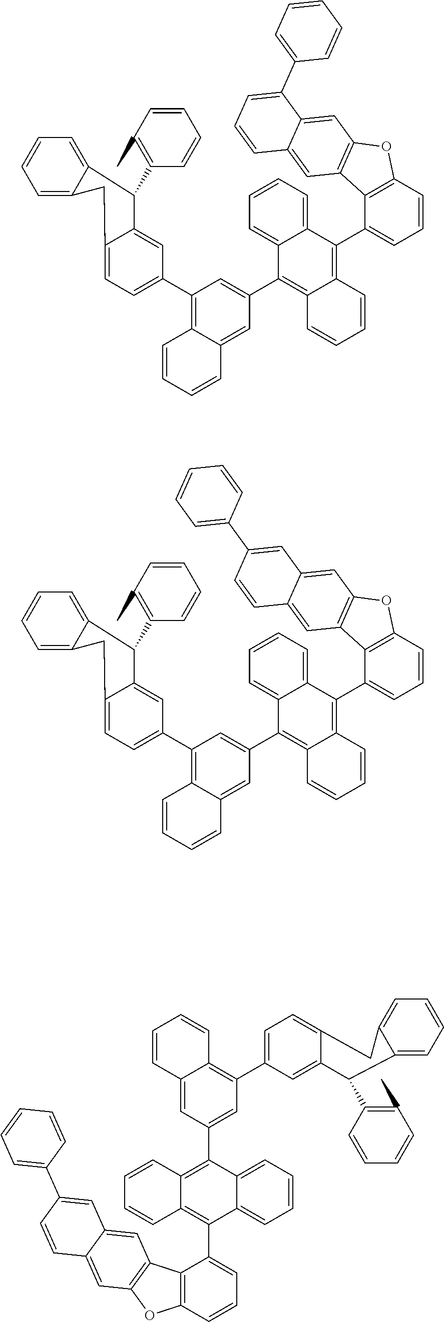

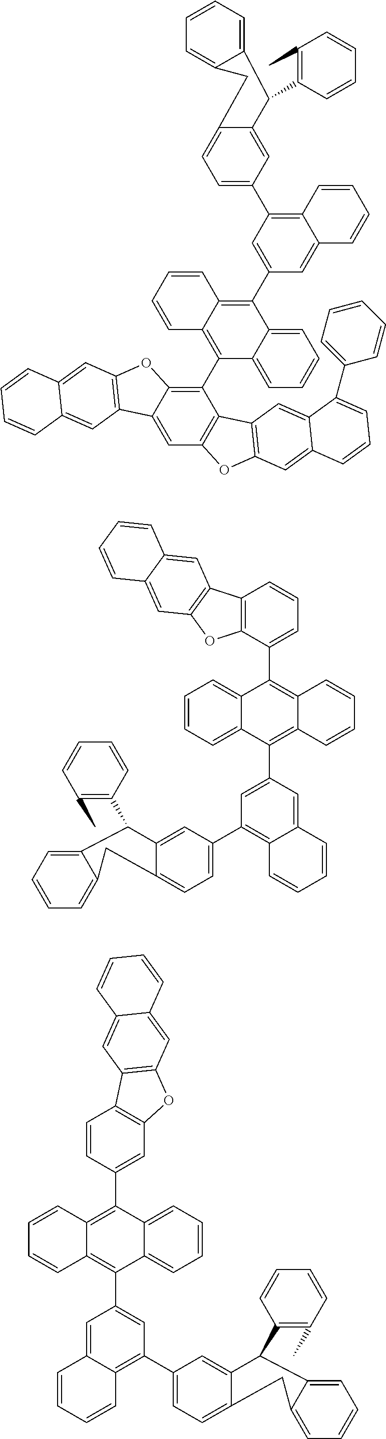

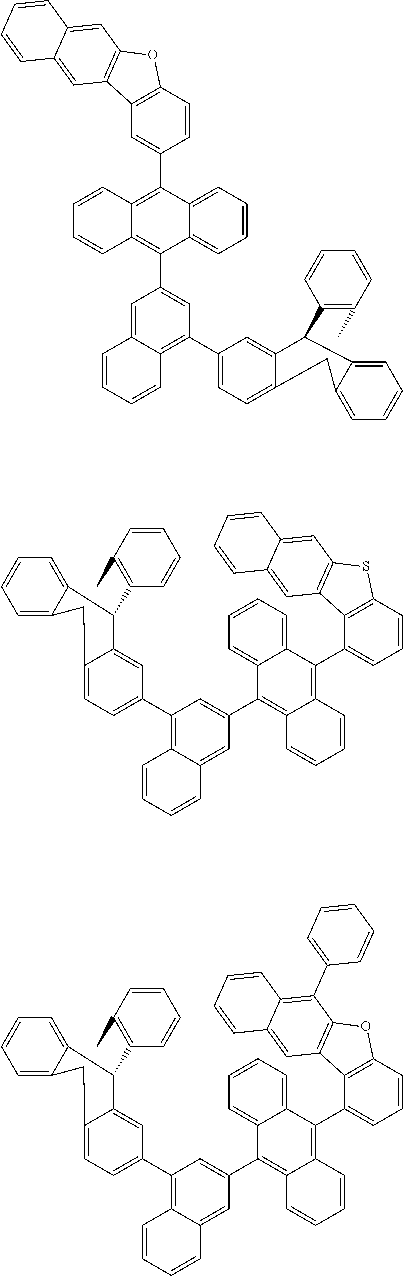

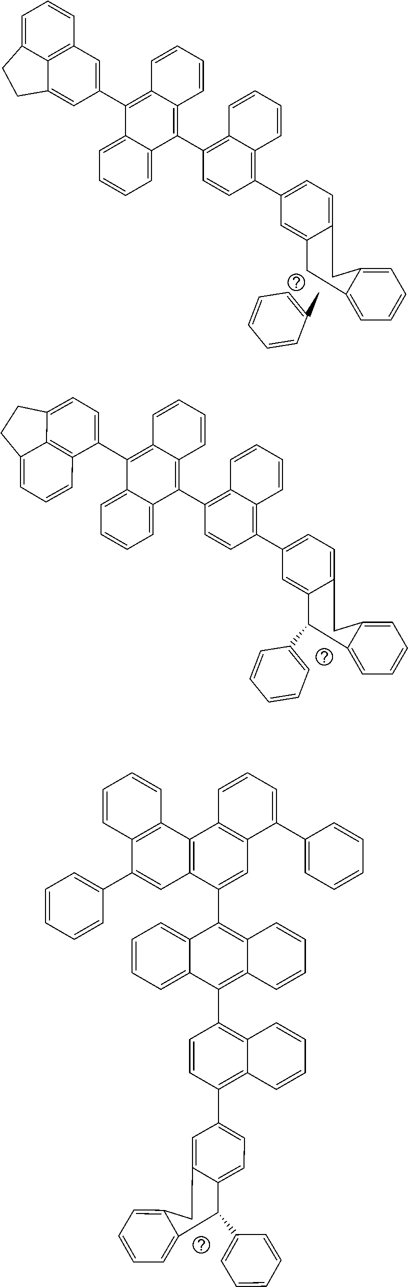

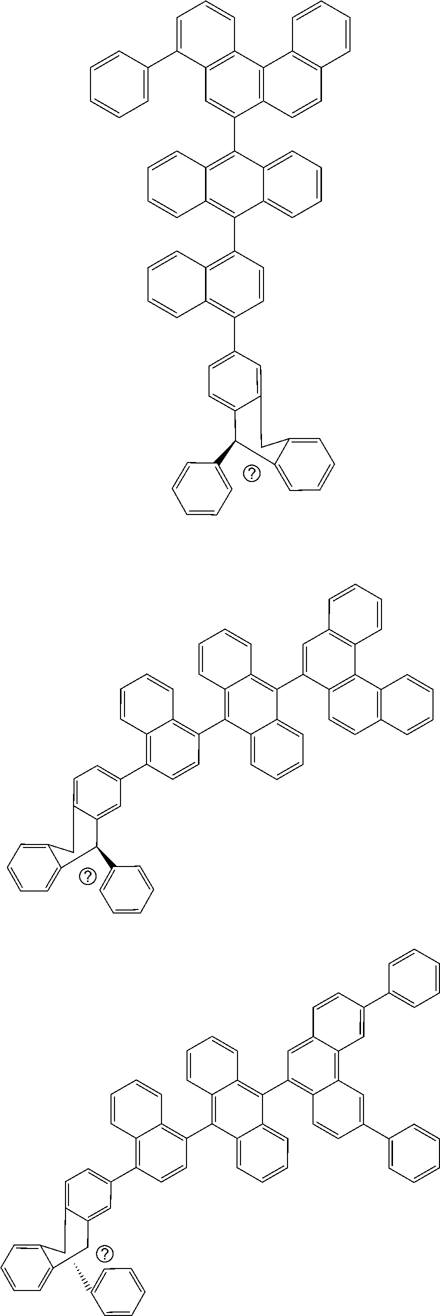

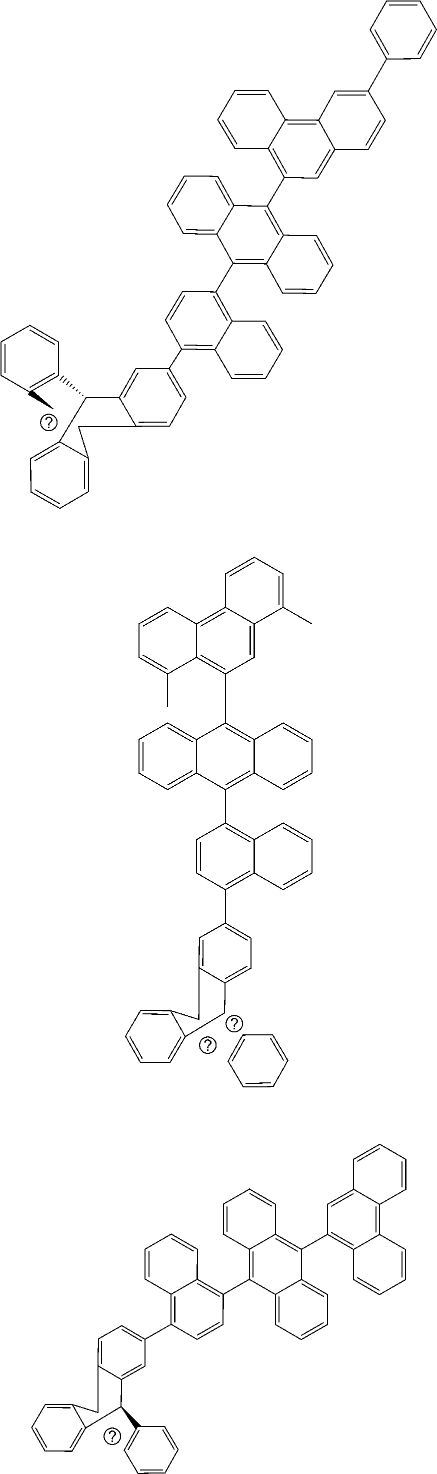

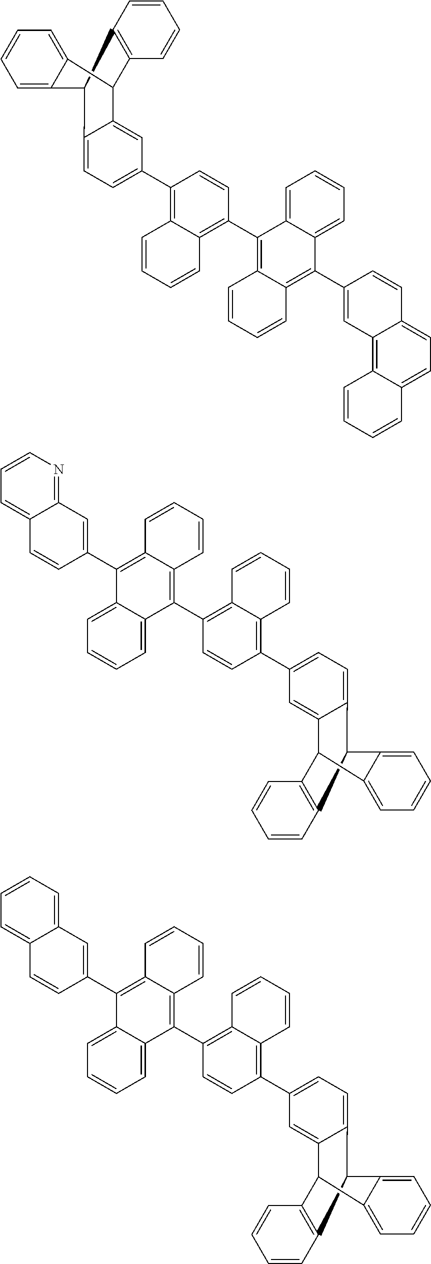

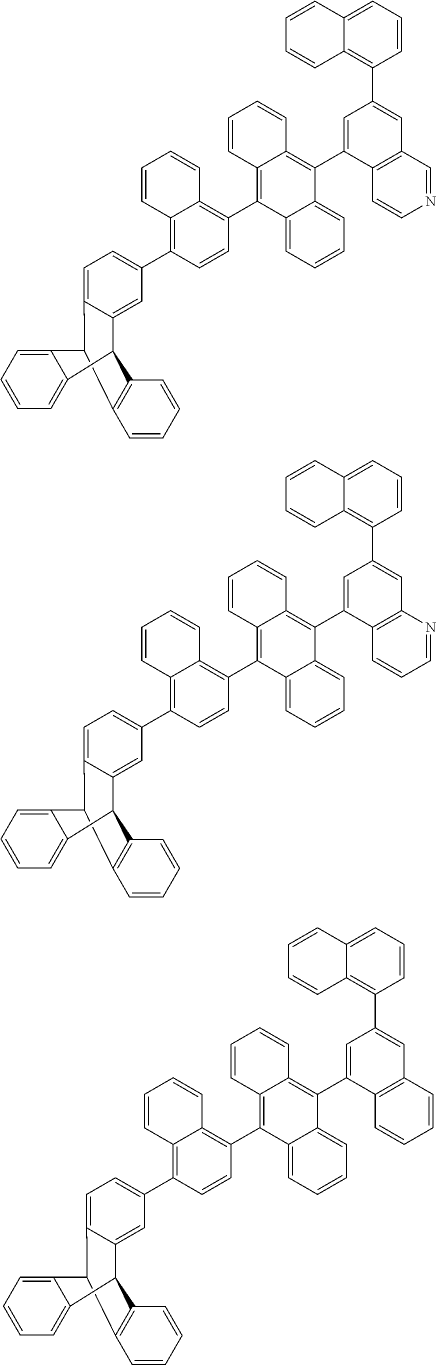

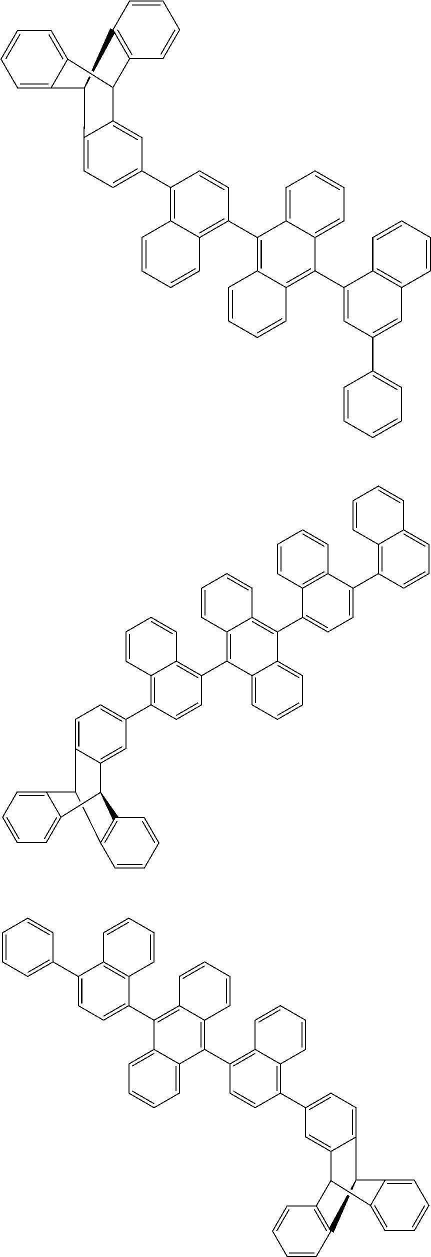

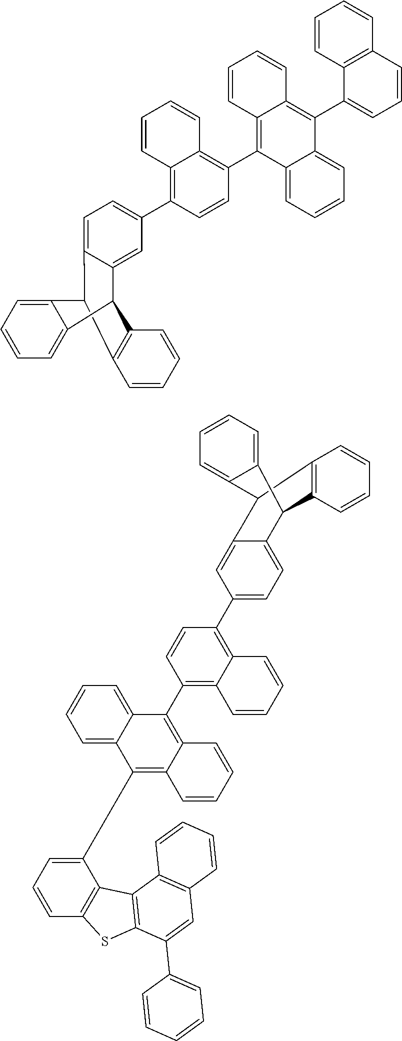

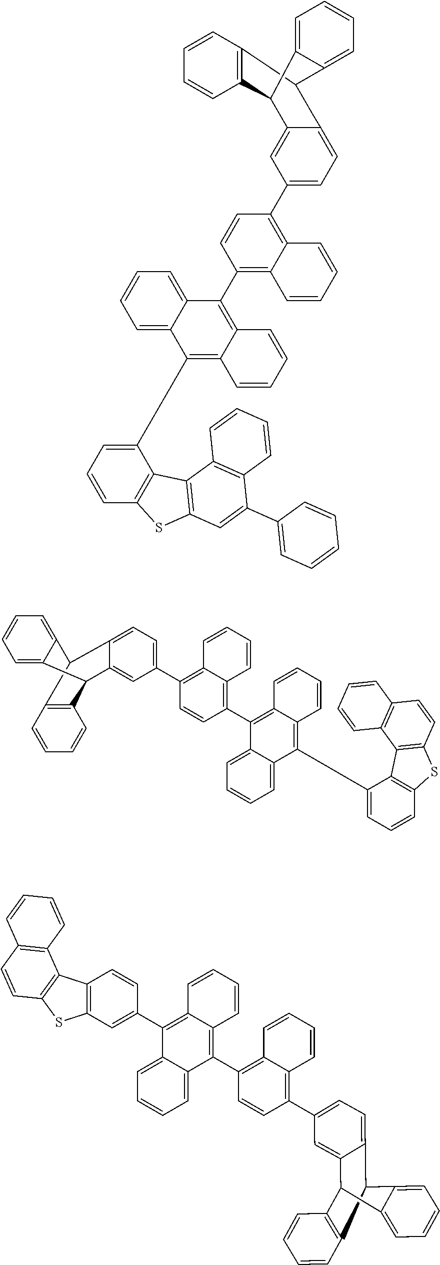

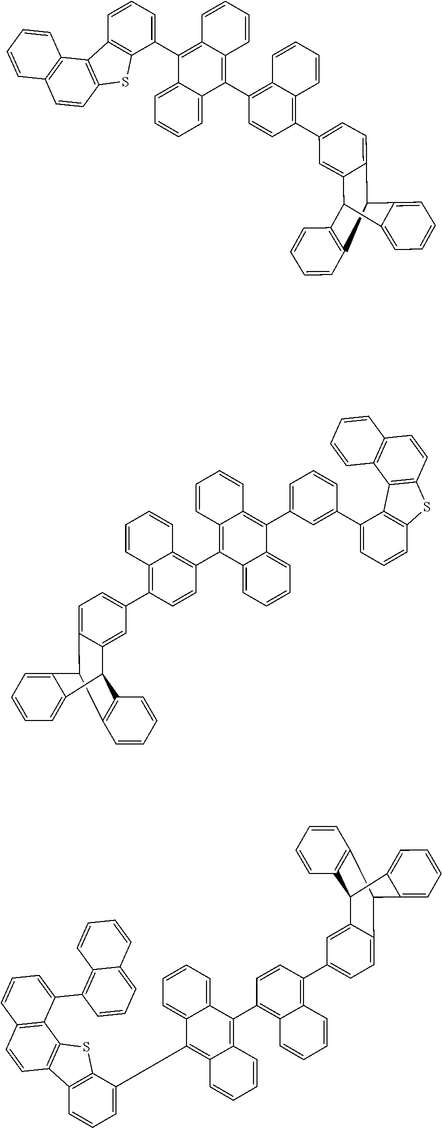

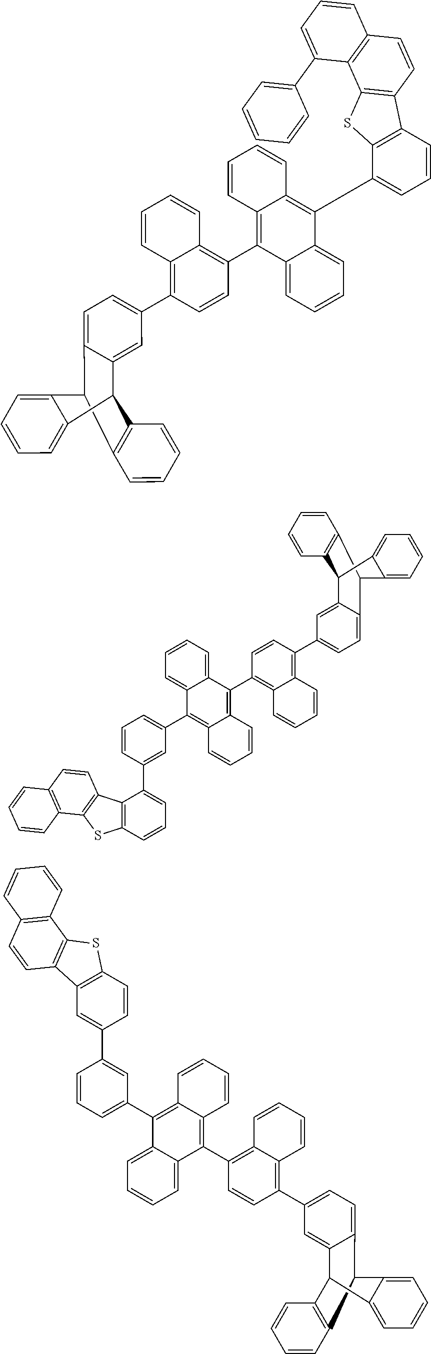

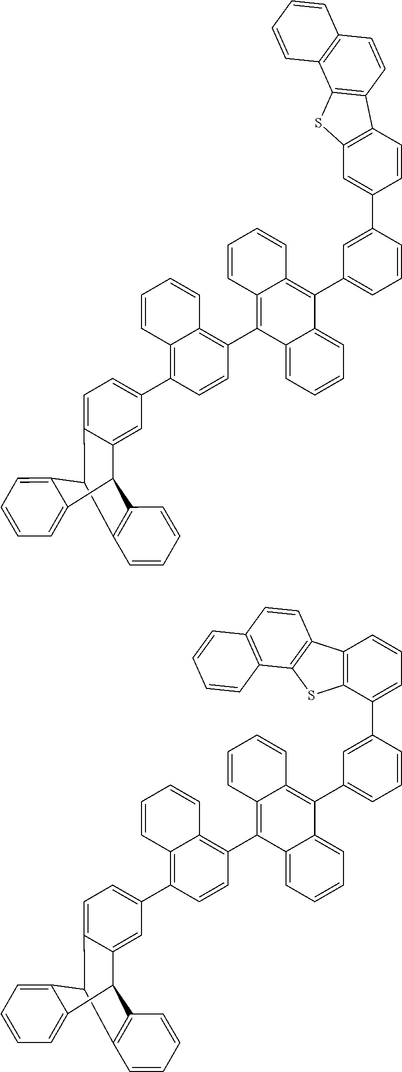

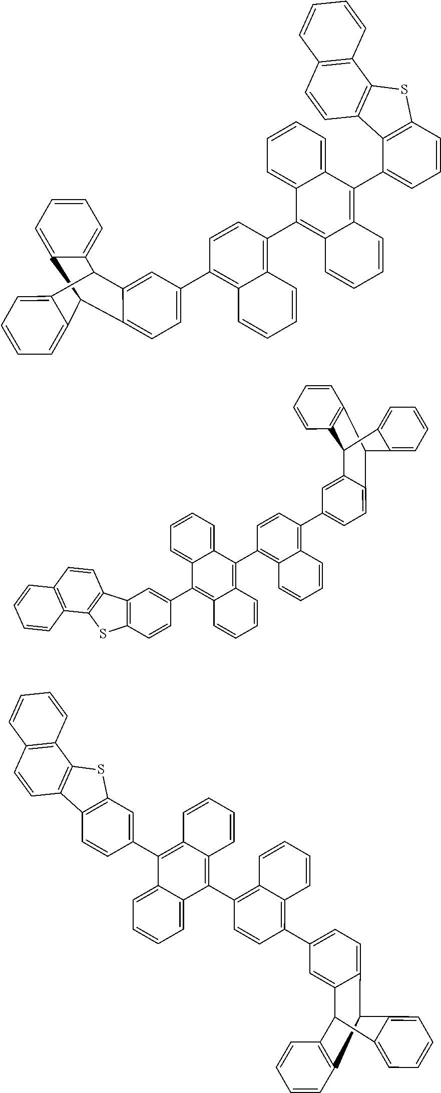

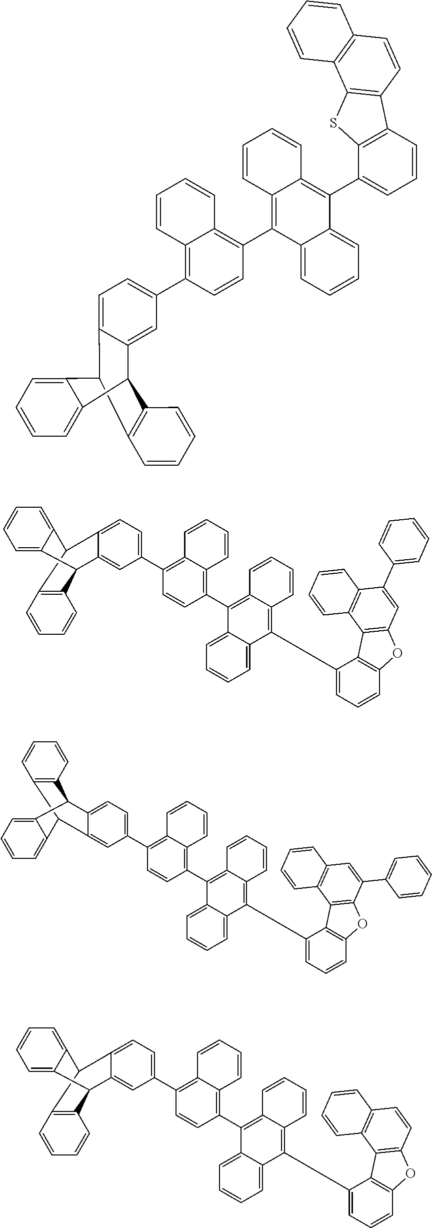

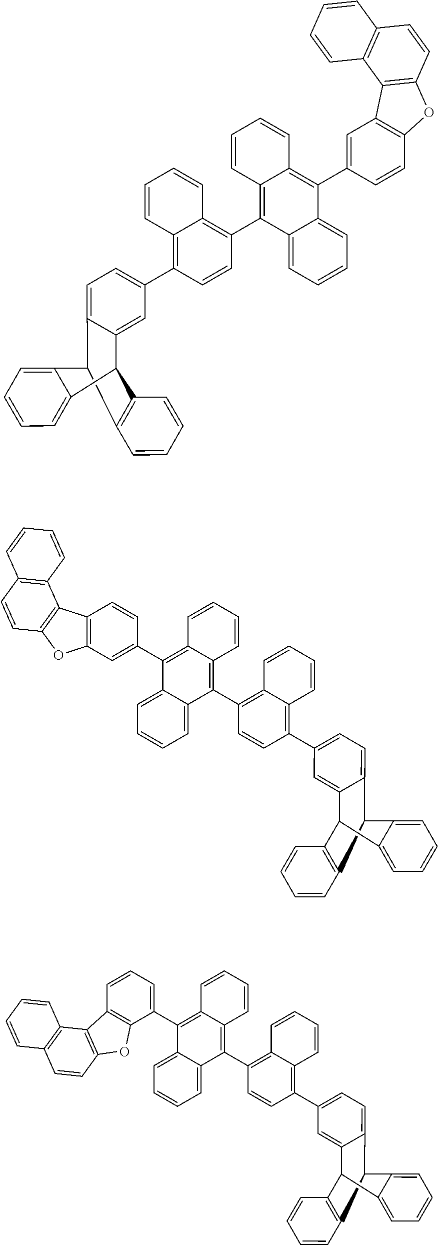

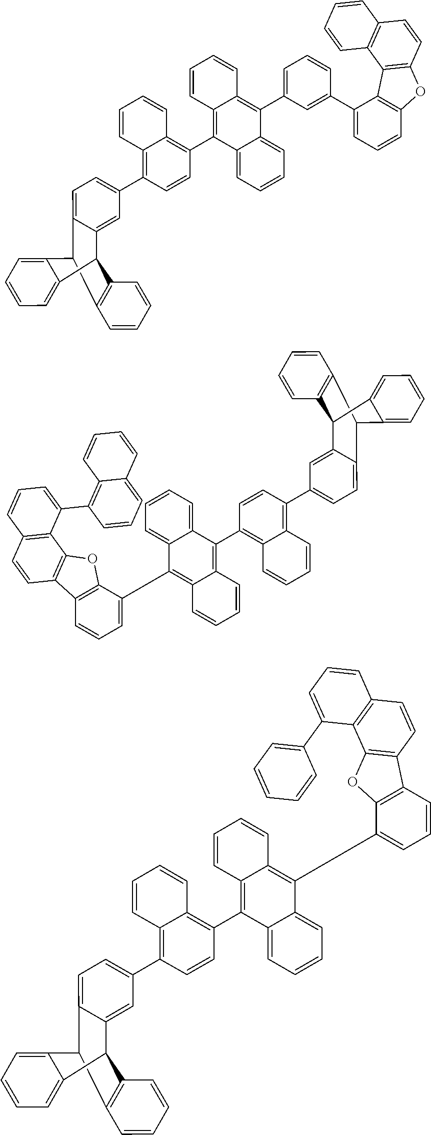

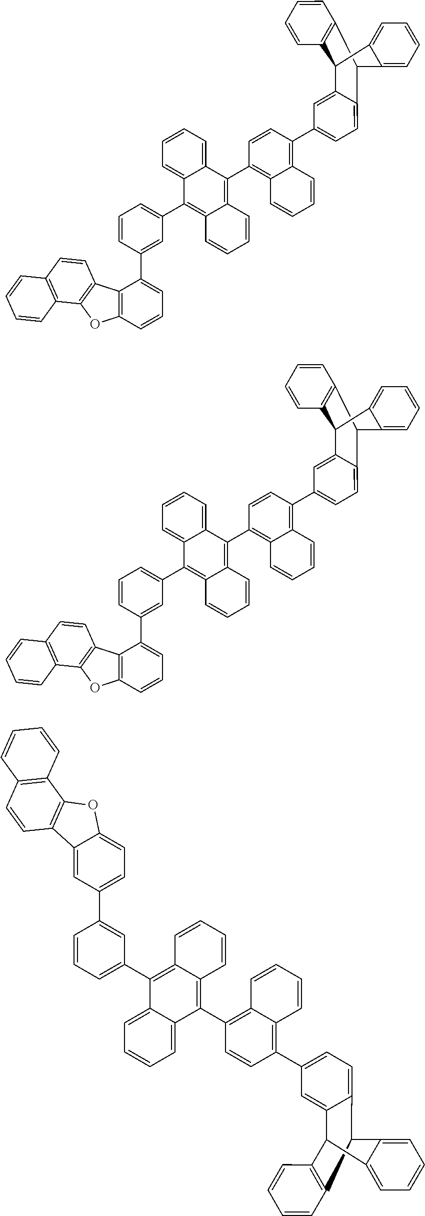

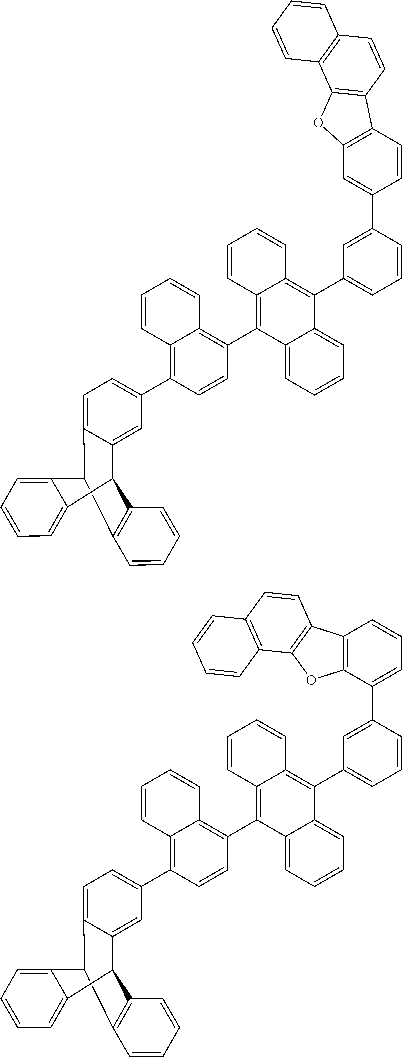

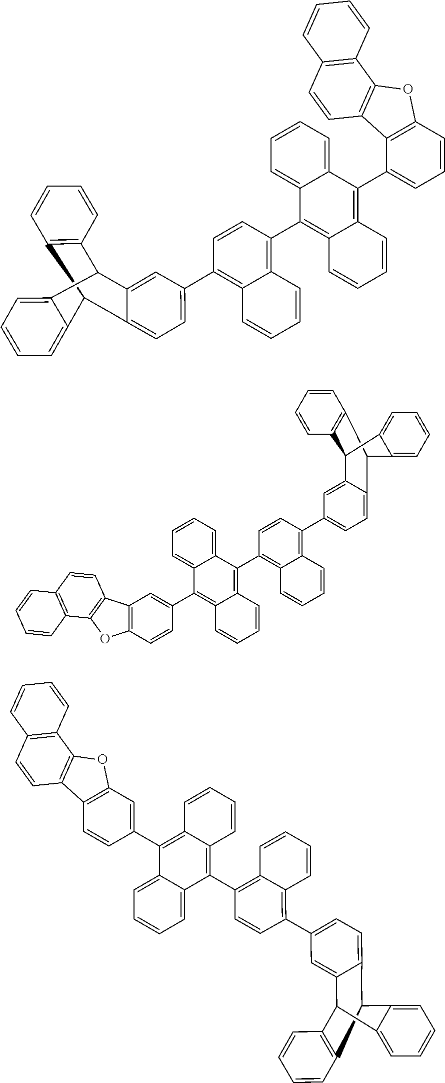

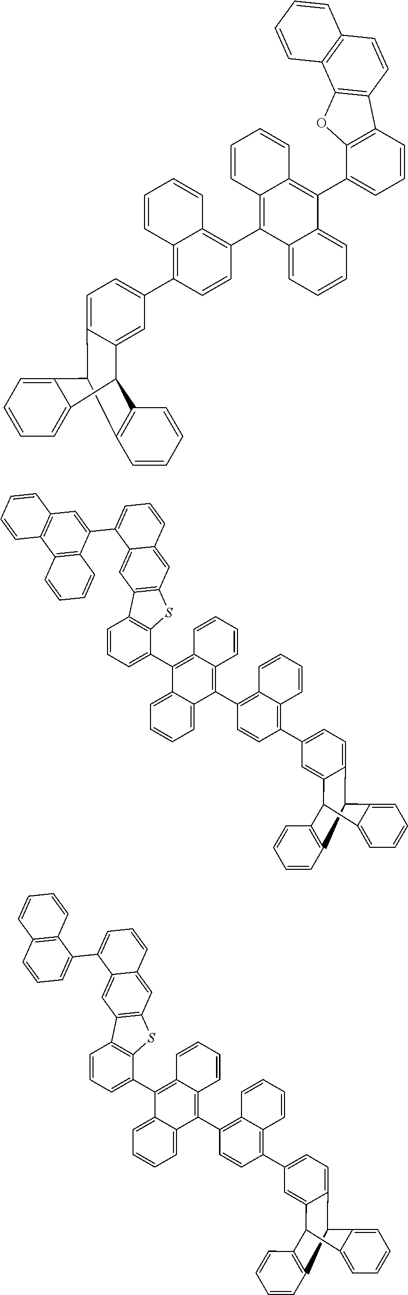

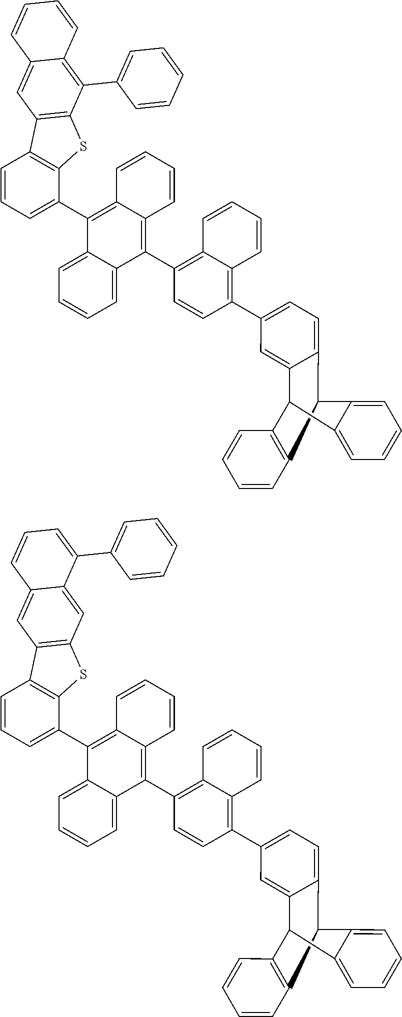

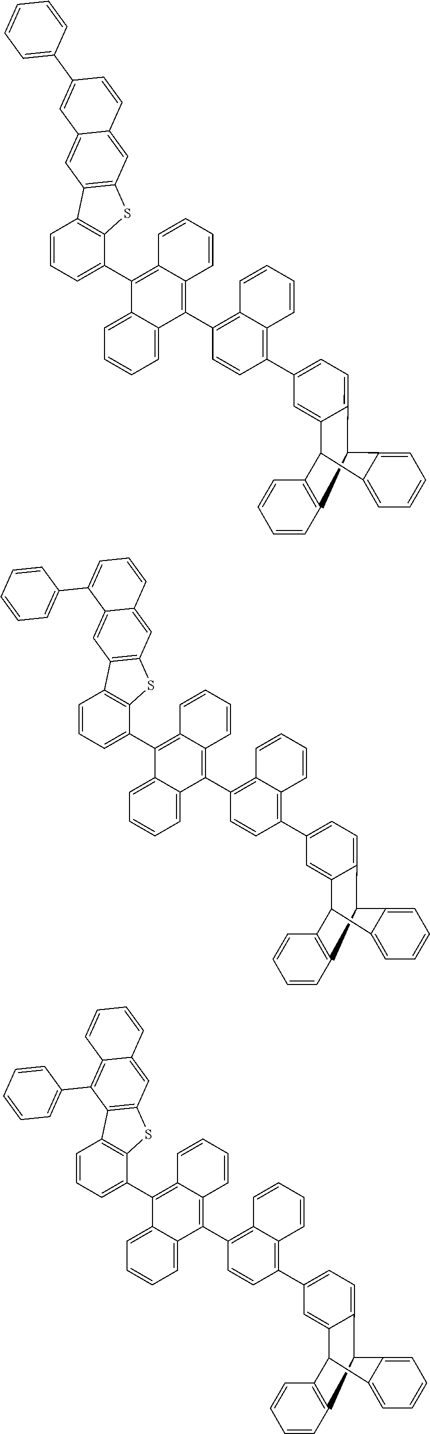

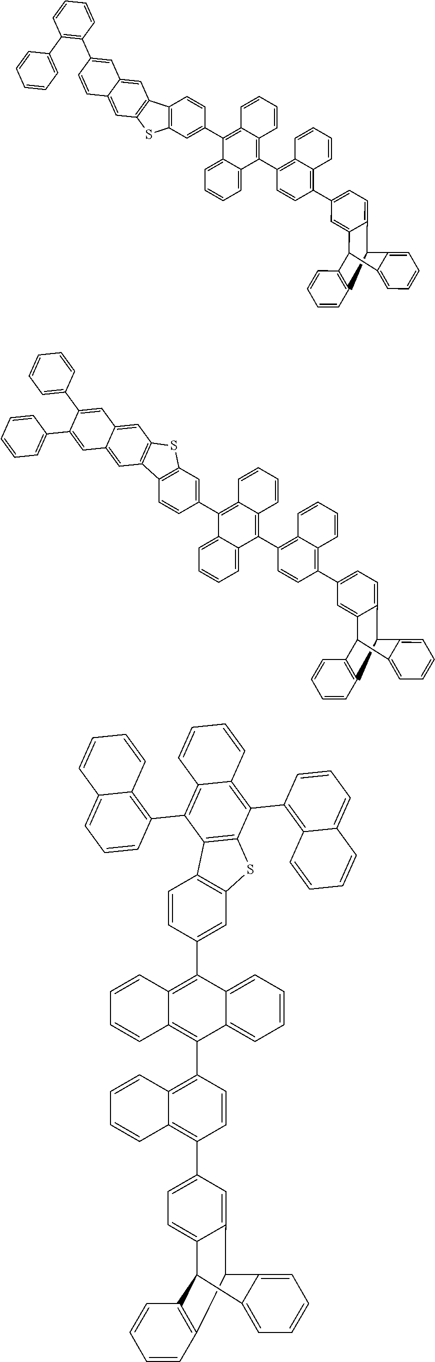

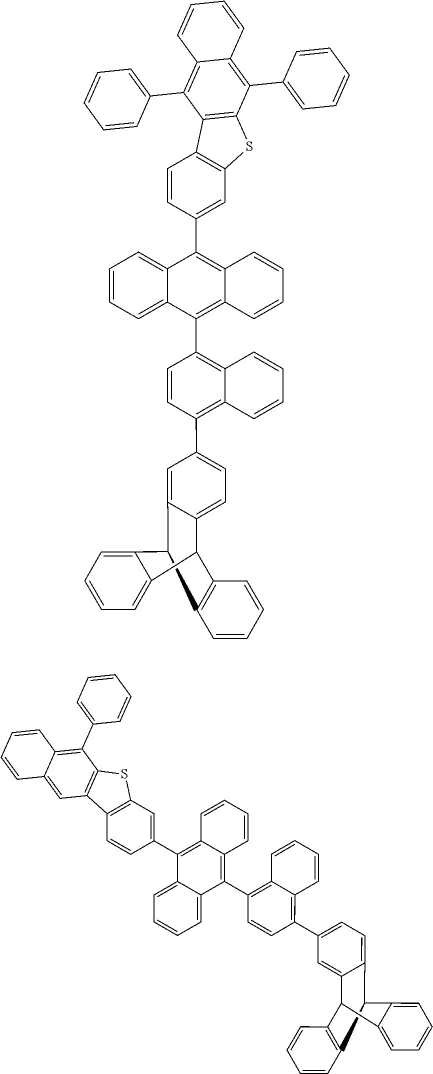

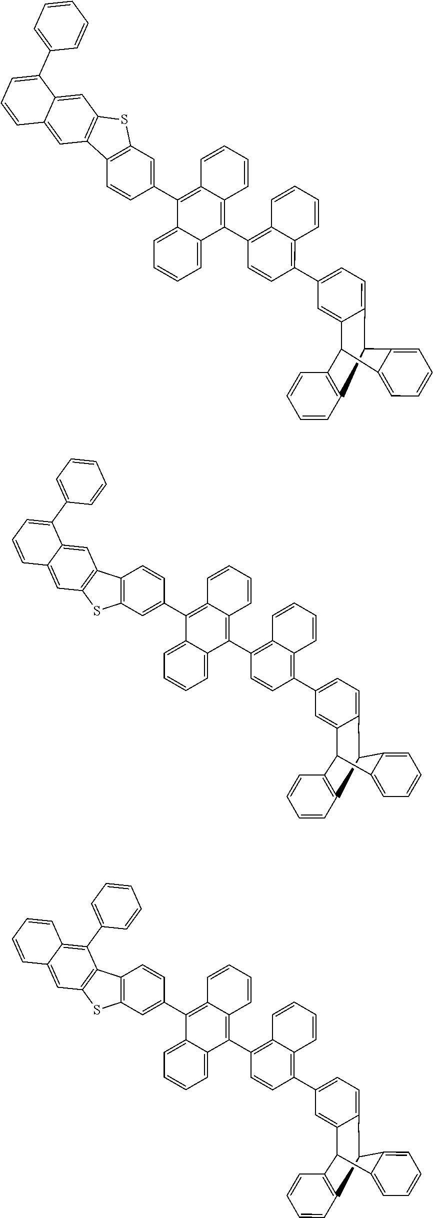

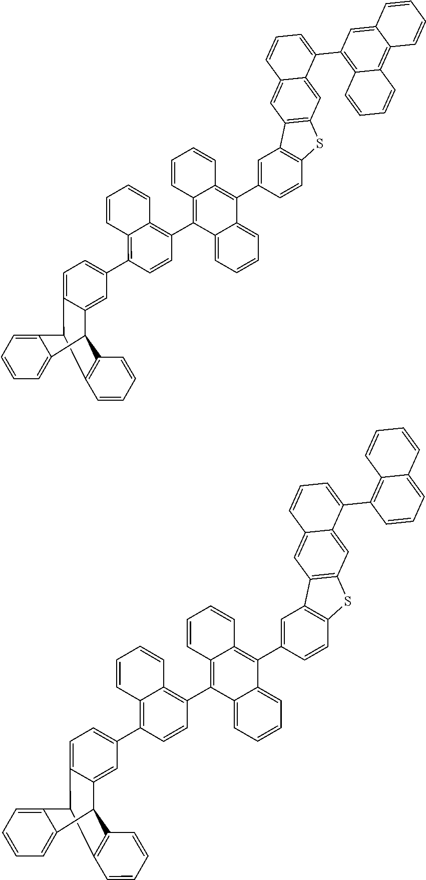

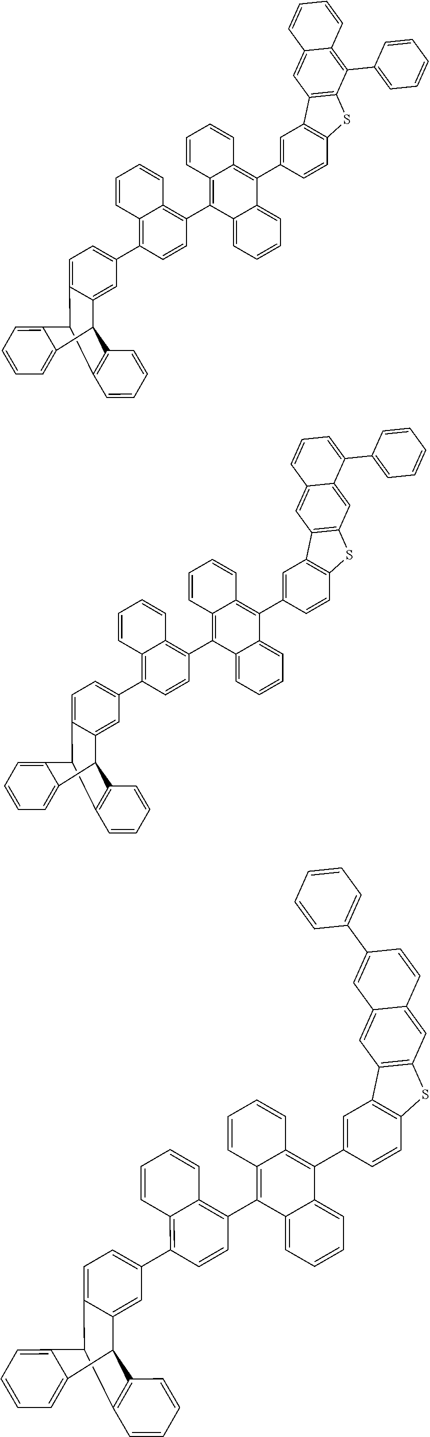

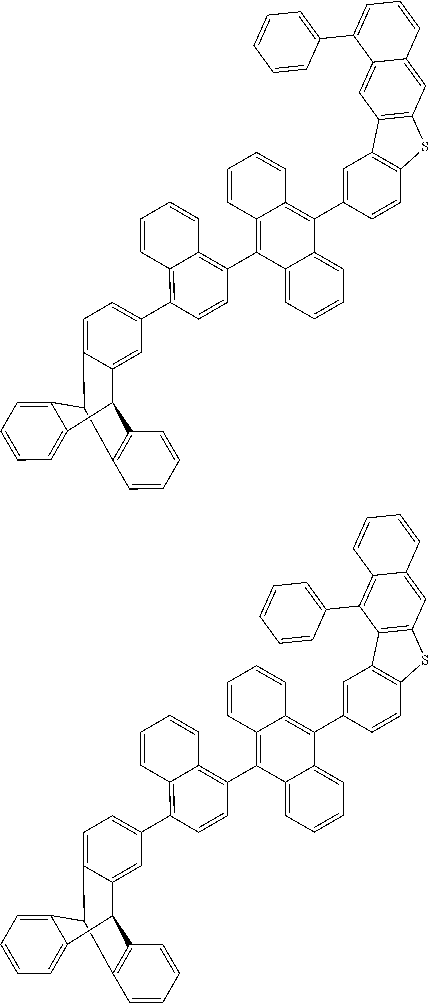

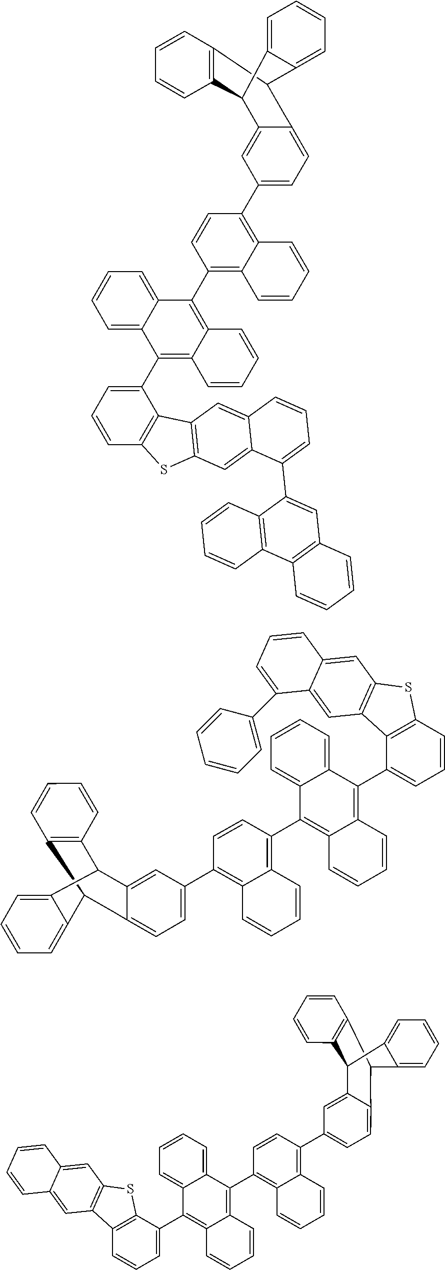

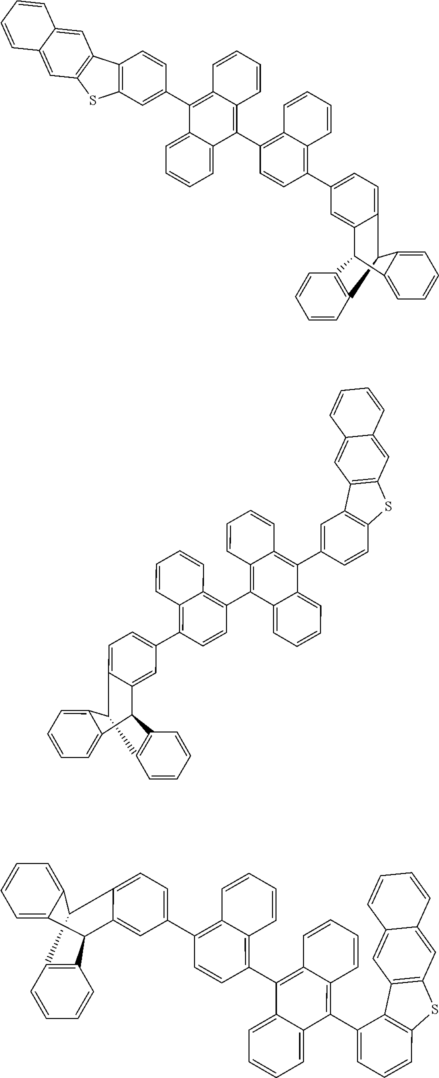 [SMILES: c1ccc2cc3c(cc2c1)sc1cc(-c2c4ccccc4c(-c4ccc(-c5ccc6c(c5)[C@H]5c7ccccc7[C@@H]6c6ccccc65)c5ccccc45)c4ccccc24)ccc13 ; c1ccc2cc3c(cc2c1)sc1ccc(-c2c4ccccc4c(-c4ccc(-c5ccc6c(c5)[C@H]5c7ccccc7[C@@H]6c6ccccc65)c5ccccc45)c4ccccc24)cc13 ; c1ccc2cc3c(cc2c1)sc1cccc(-c2c4ccccc4c(-c4ccc(-c5ccc6c(c5)[C@H]5c7ccccc7[C@@H]6c6ccccc65)c5ccccc45)c4ccccc24)c13]

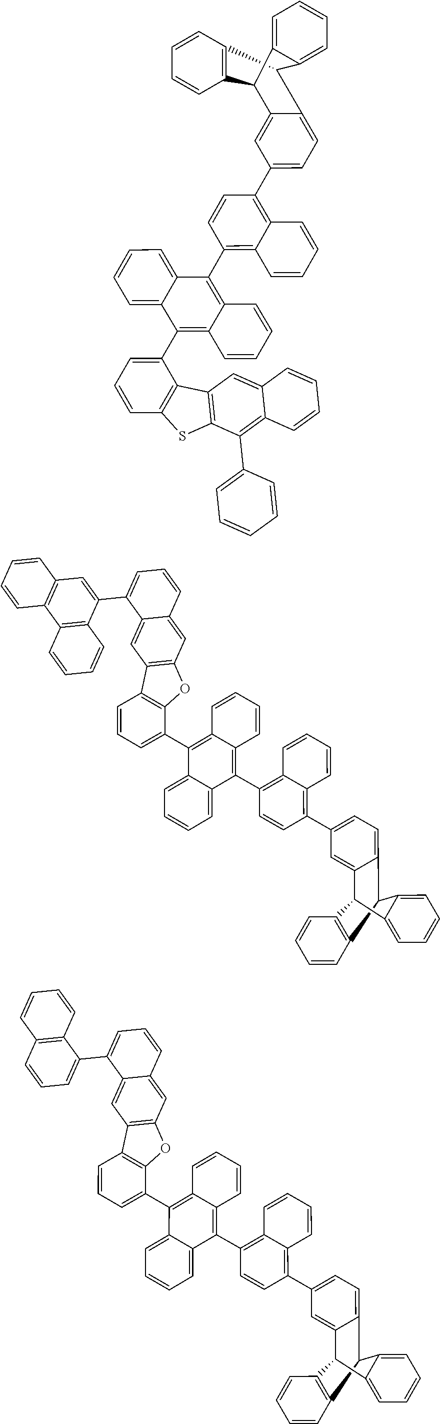 [SMILES: c1ccc(-c2c3ccccc3cc3c2sc2cccc(-c4c5ccccc5c(-c5ccc(-c6ccc7c(c6)[C@H]6c8ccccc8[C@@H]7c7ccccc76)c6ccccc56)c5ccccc45)c23)cc1 ; c1cc(-c2cc3ccccc3c3ccccc23)c2cc3c(cc2c1)oc1c(-c2c4ccccc4c(-c4ccc(-c5ccc6c(c5)[C@H]5c7ccccc7[C@@H]6c6ccccc65)c5ccccc45)c4ccccc24)cccc13 ; c1ccc2c(-c3cccc4cc5oc6c(-c7c8ccccc8c(-c8ccc(-c9ccc%10c(c9)[C@H]9c%11ccccc%11[C@@H]%10c%10ccccc%109)c9ccccc89)c8ccccc78)cccc6c5cc34)cccc2c1]

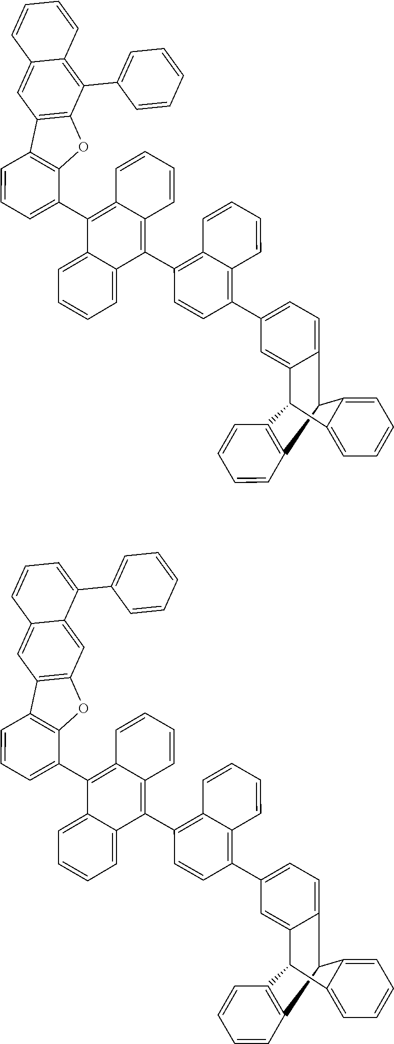 [SMILES: c1ccc(-c2c3ccccc3cc3c2oc2c(-c4c5ccccc5c(-c5ccc(-c6ccc7c(c6)[C@H]6c8ccccc8[C@@H]7c7ccccc76)c6ccccc56)c5ccccc45)cccc23)cc1 ; c1ccc(-c2cccc3cc4c(cc23)oc2c(-c3c5ccccc5c(-c5ccc(-c6ccc7c(c6)[C@H]6c8ccccc8[C@@H]7c7ccccc76)c6ccccc56)c5ccccc35)cccc24)cc1]

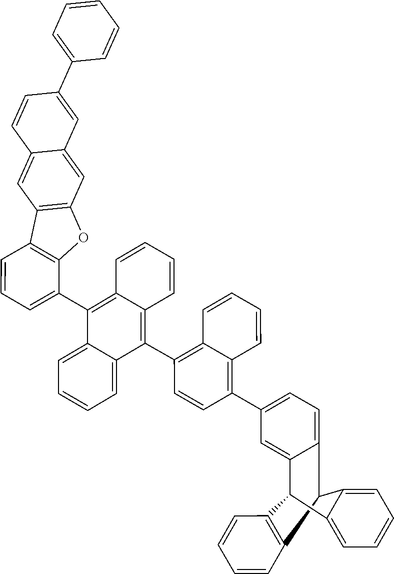 [SMILES: c1ccc(-c2ccc3cc4c(cc3c2)oc2c(-c3c5ccccc5c(-c5ccc(-c6ccc7c(c6)[C@H]6c8ccccc8[C@@H]7c7ccccc76)c6ccccc56)c5ccccc35)cccc24)cc1]

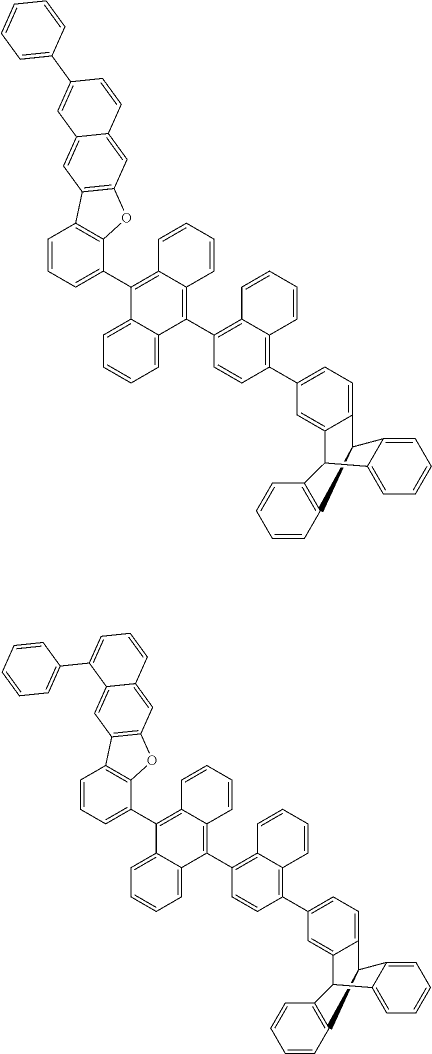

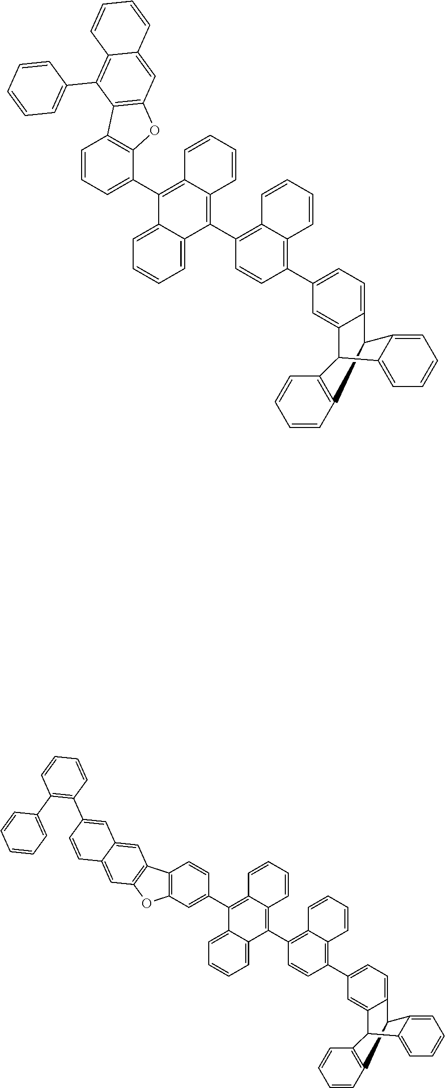

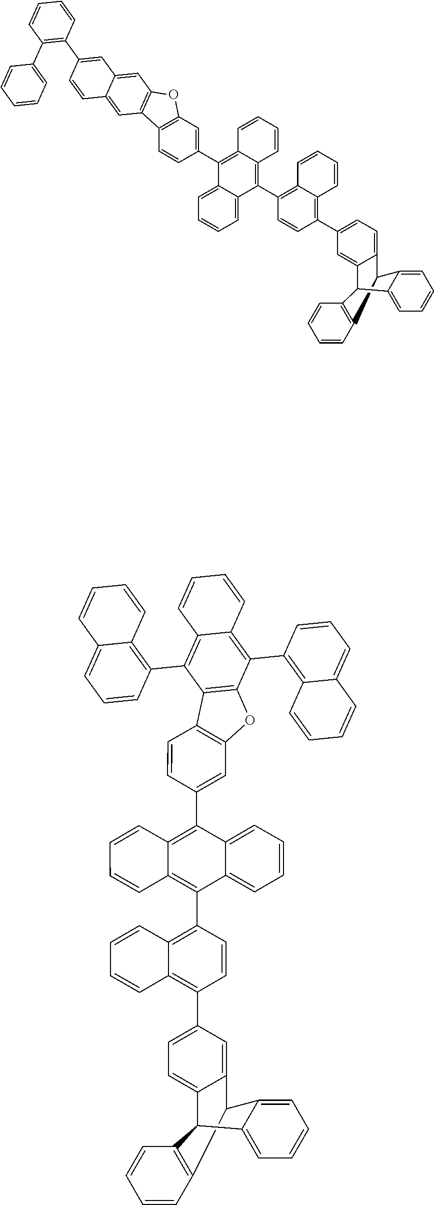

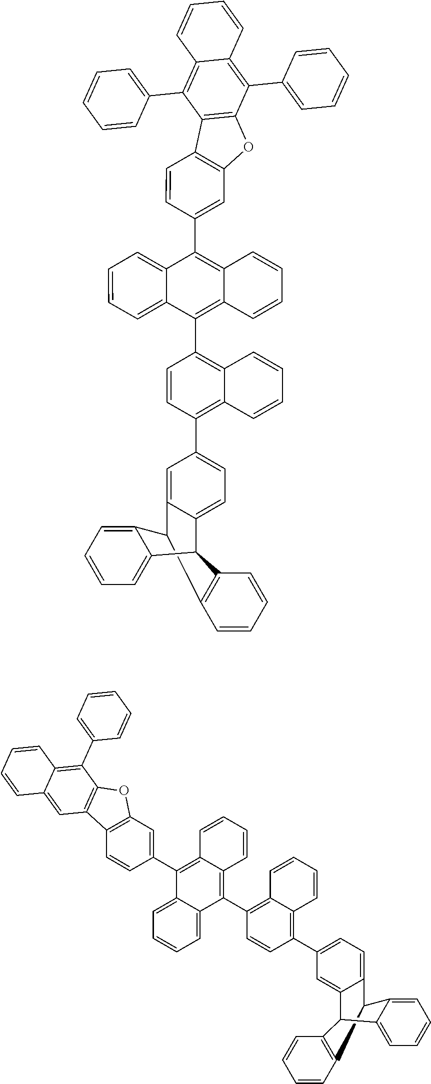

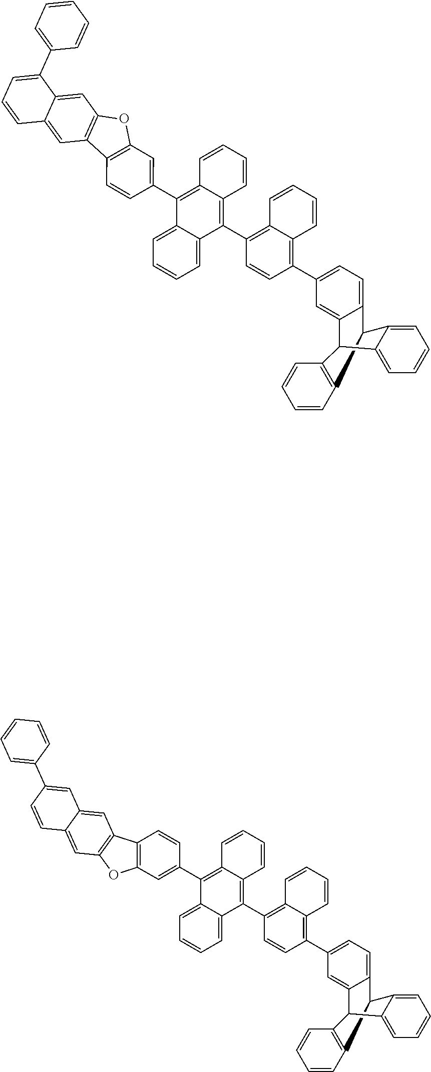

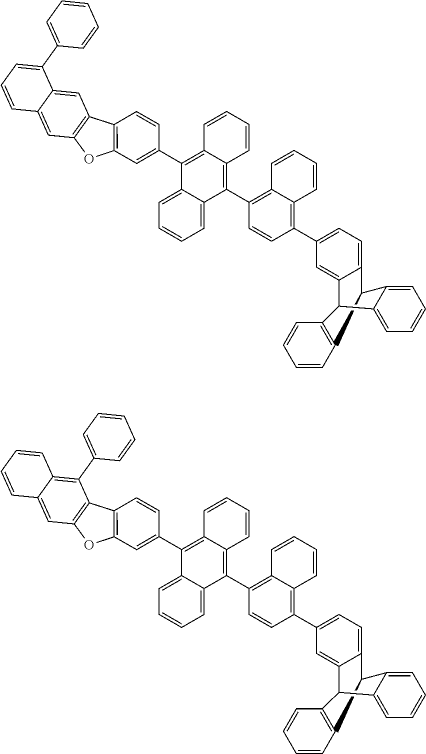

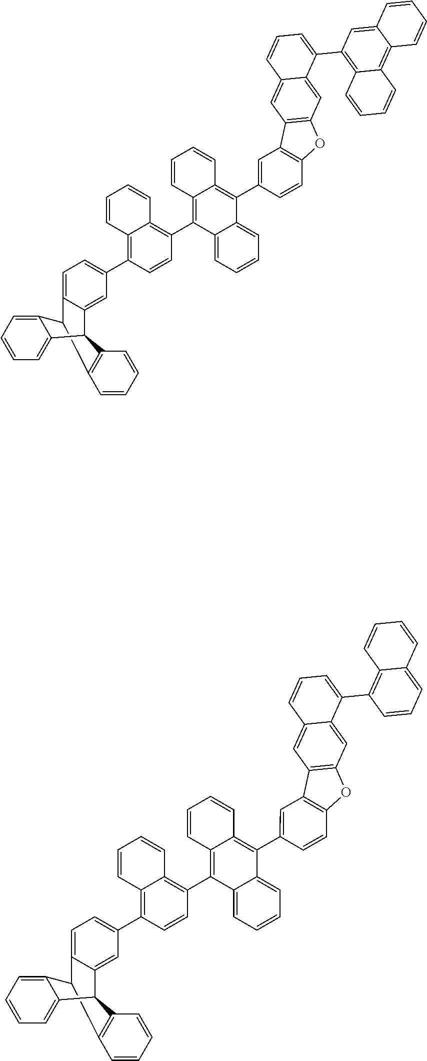

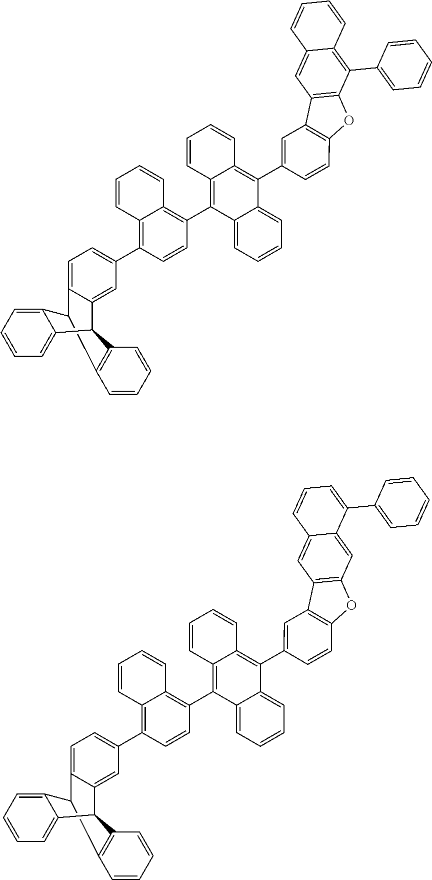

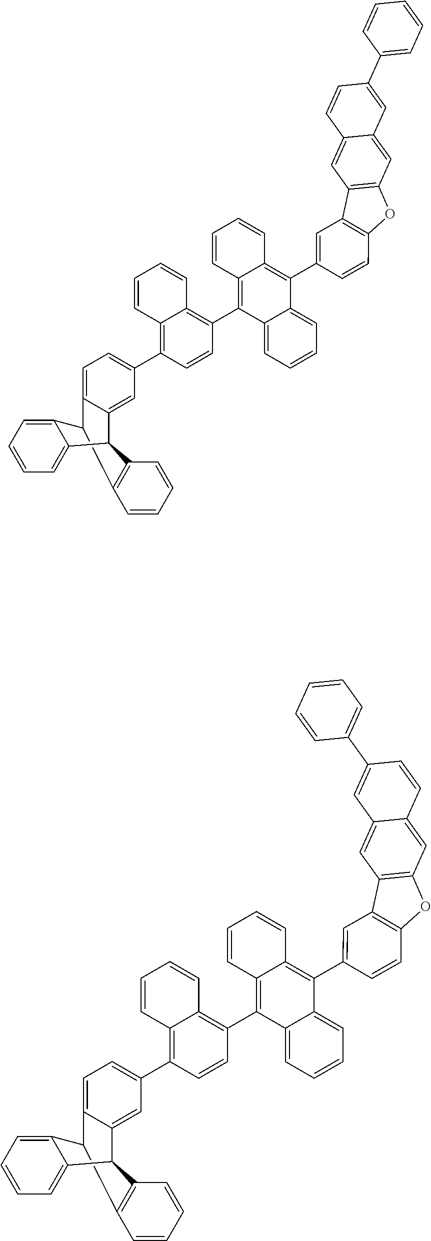

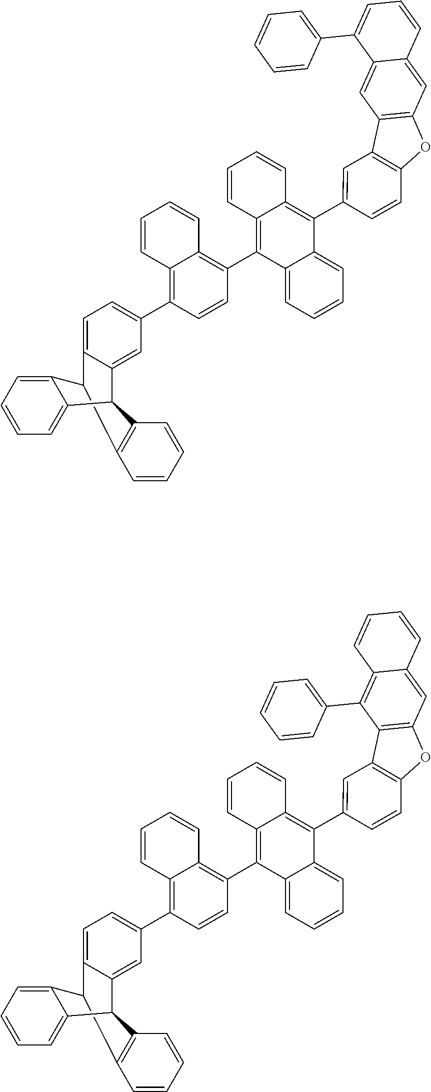

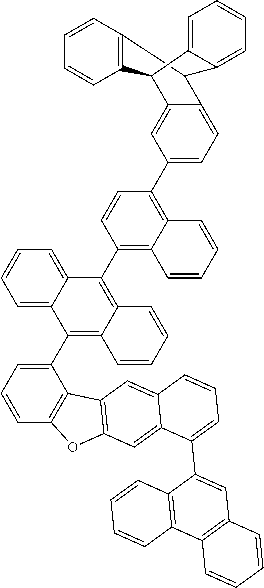

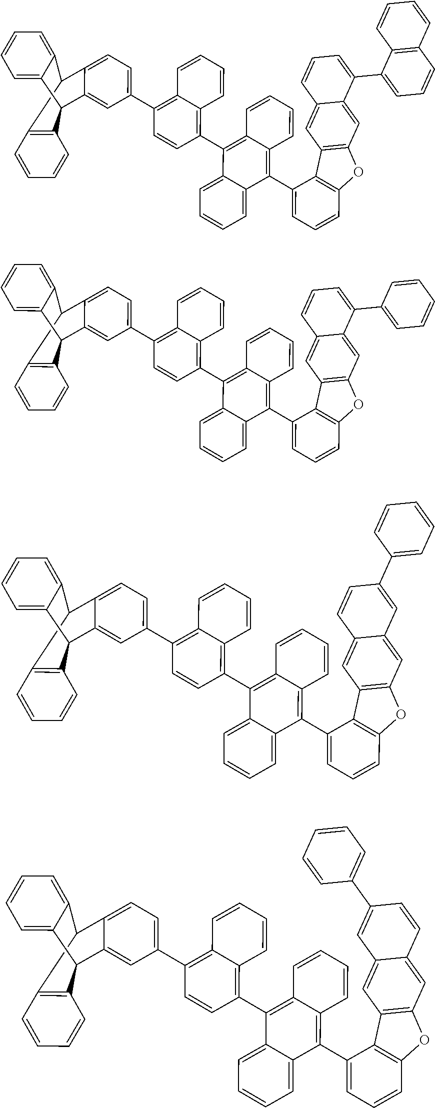

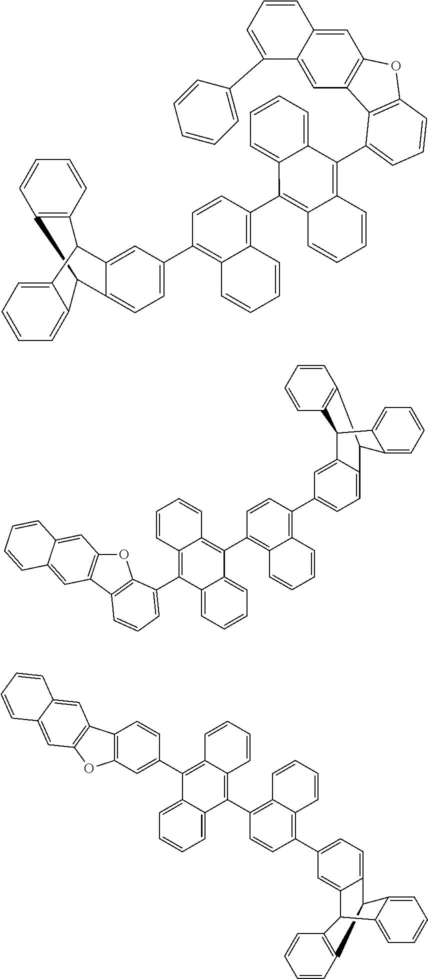

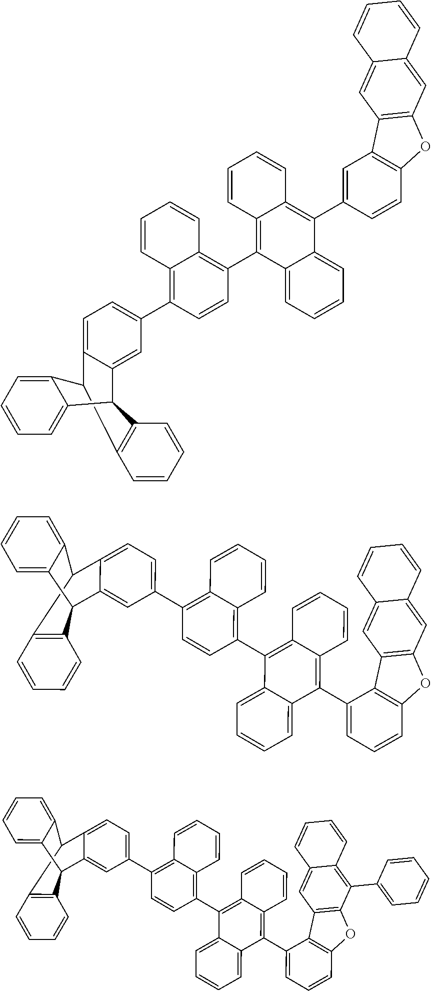

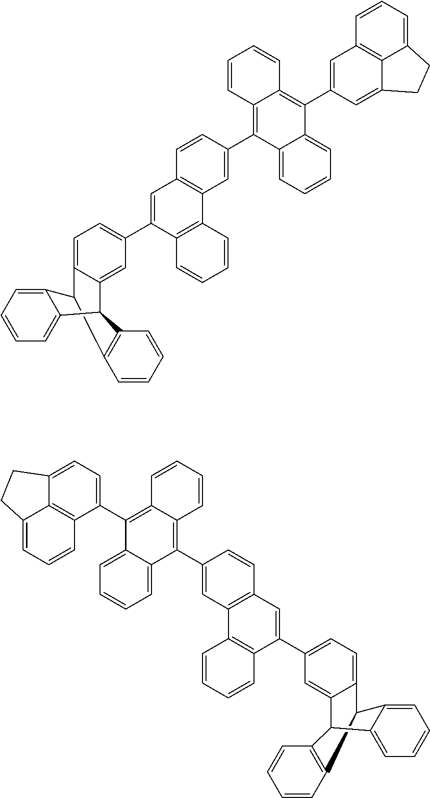

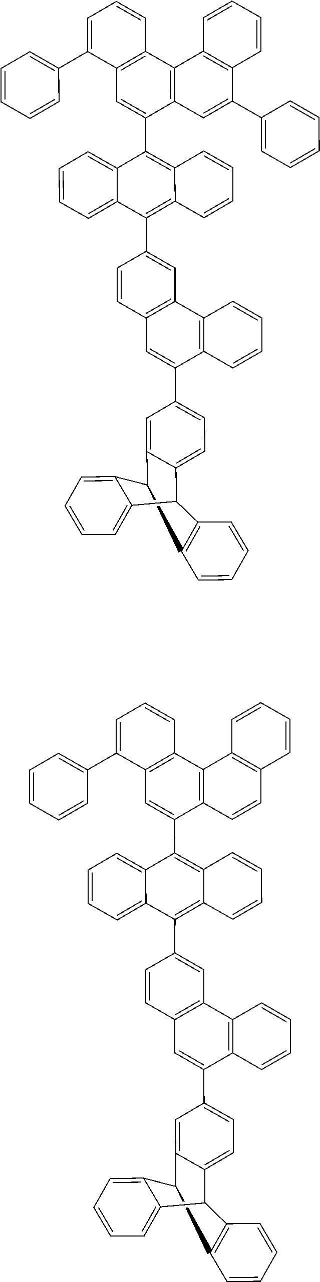

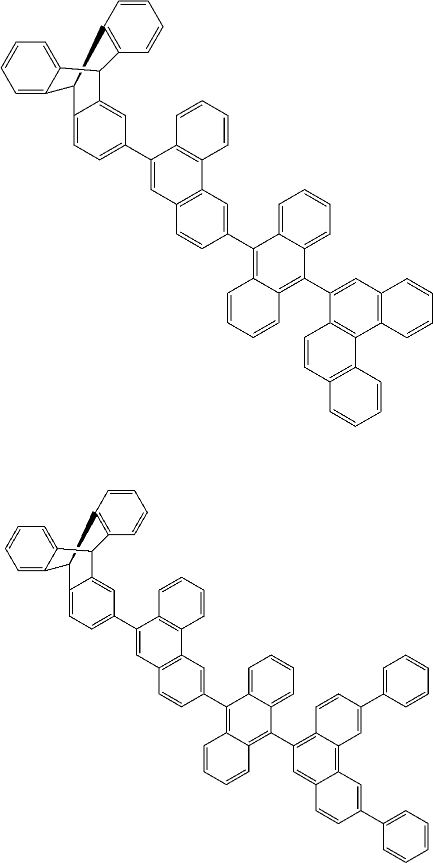

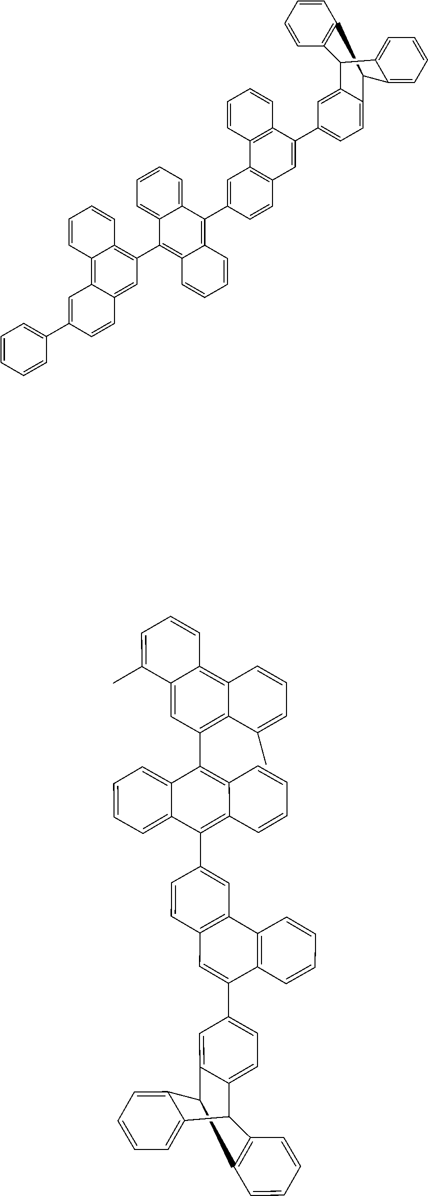

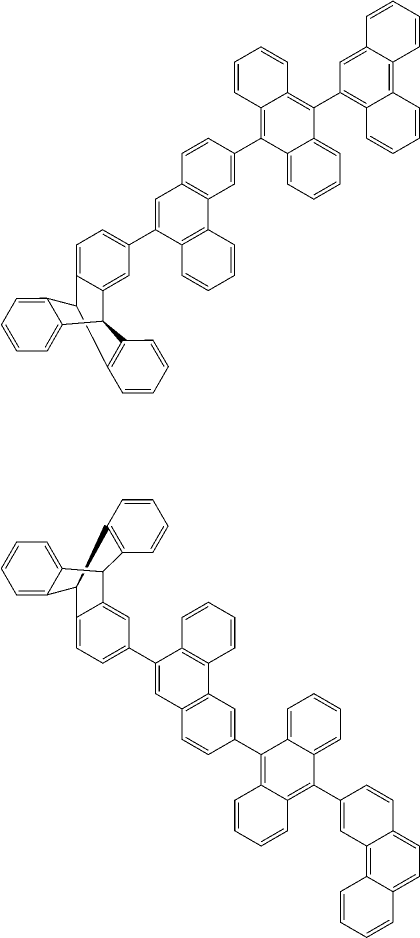

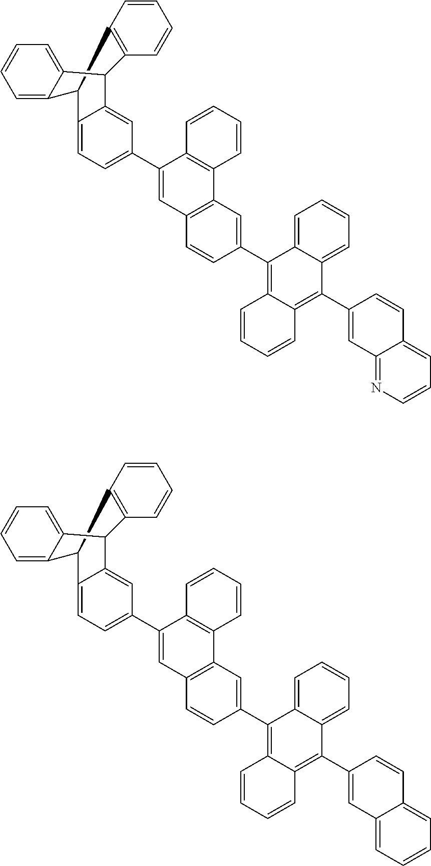

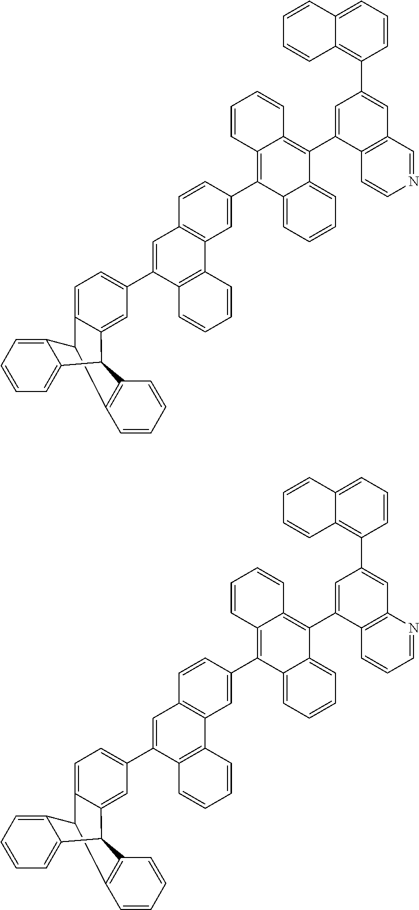

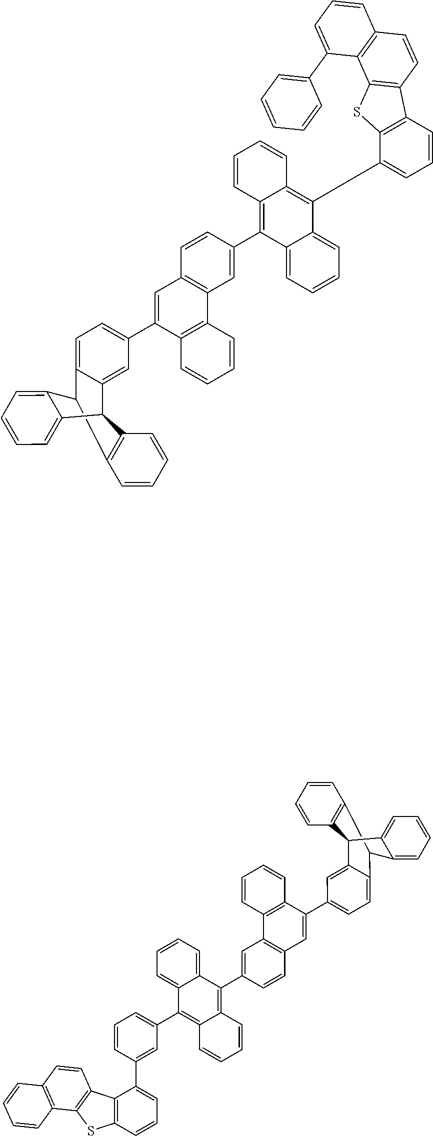

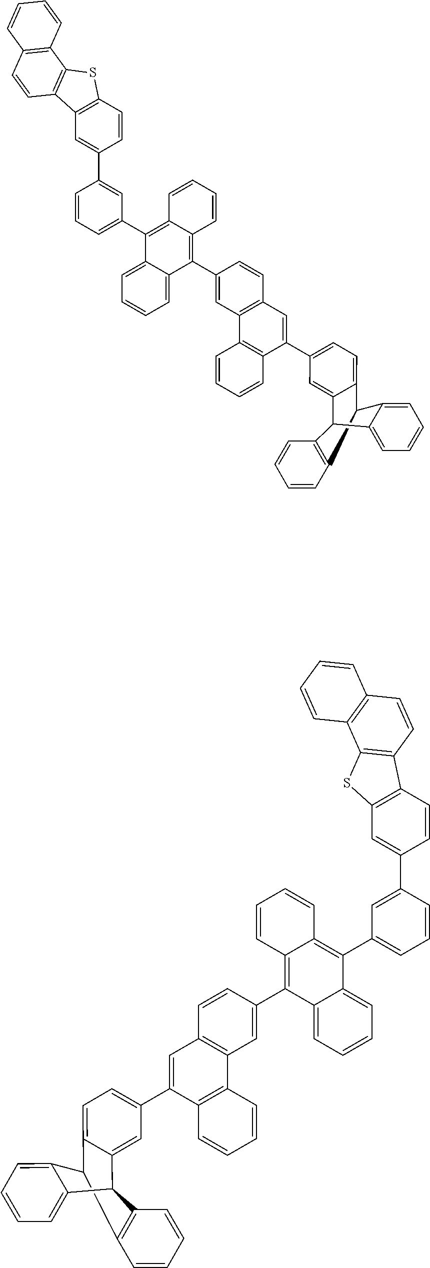

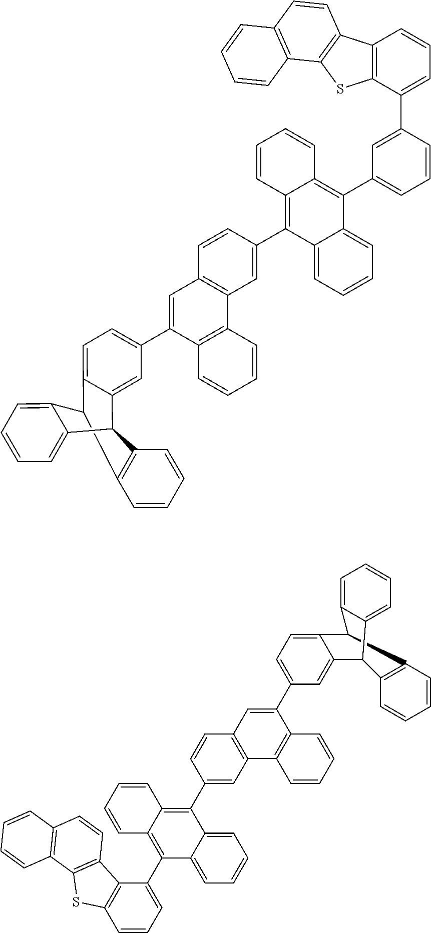

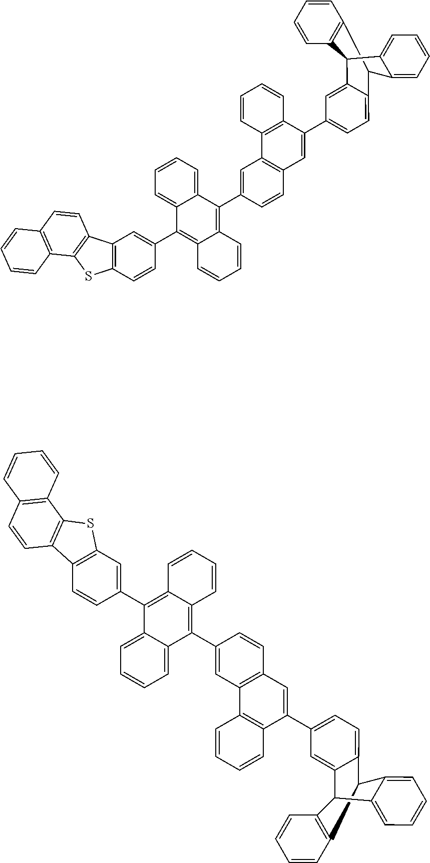

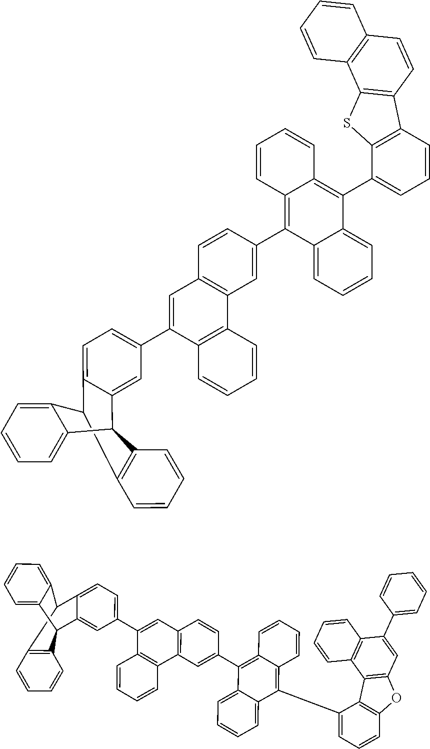

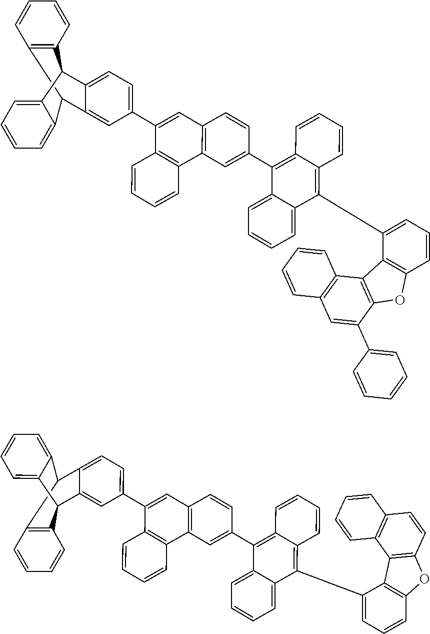

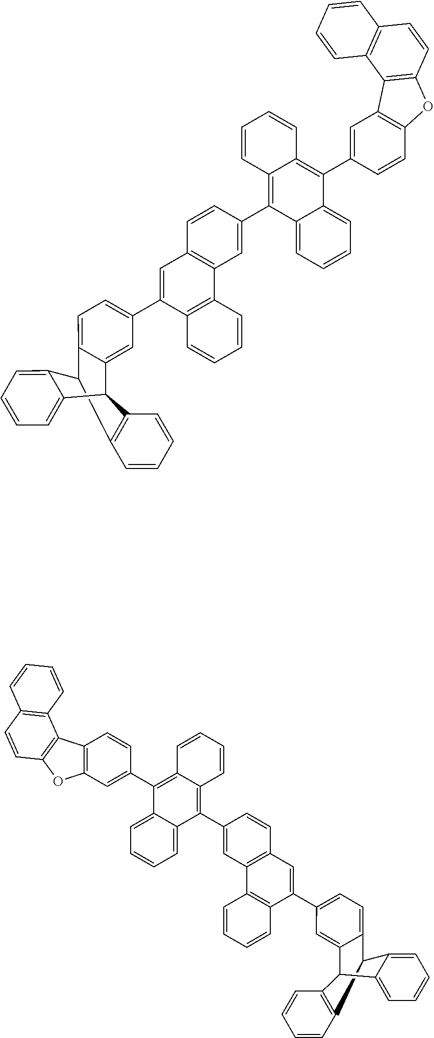

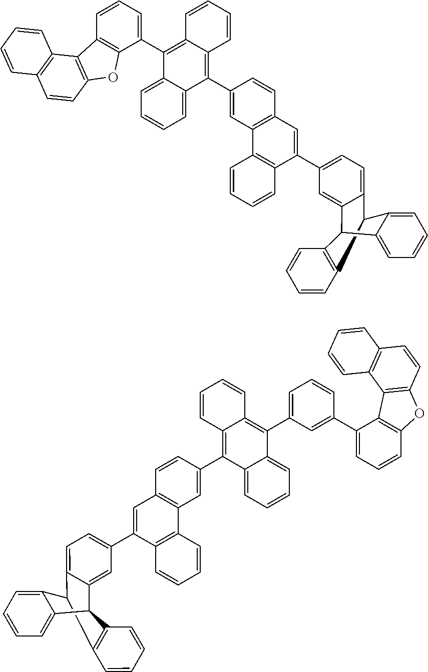

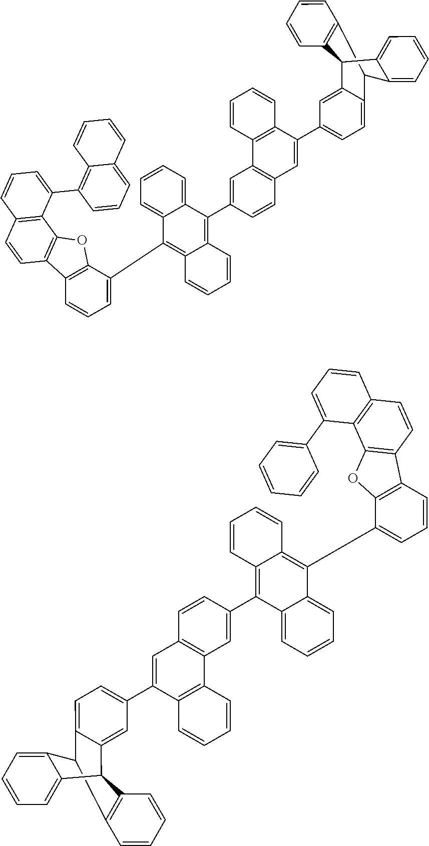

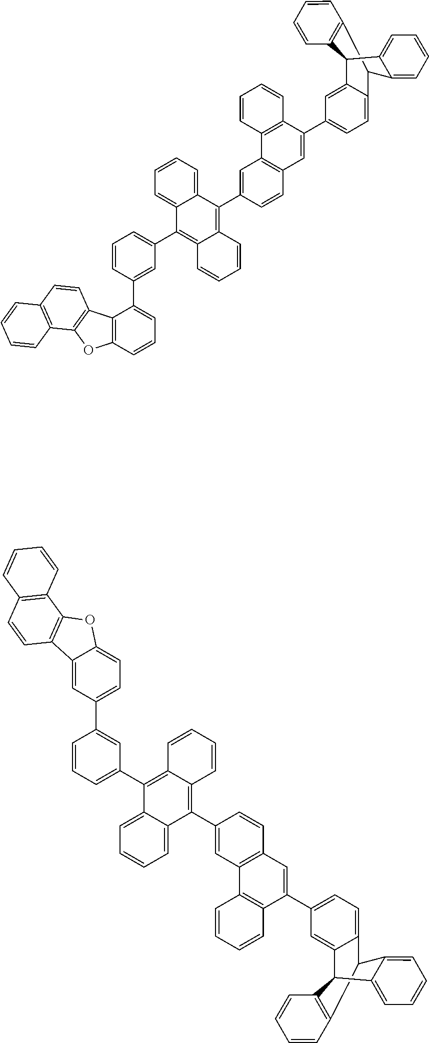

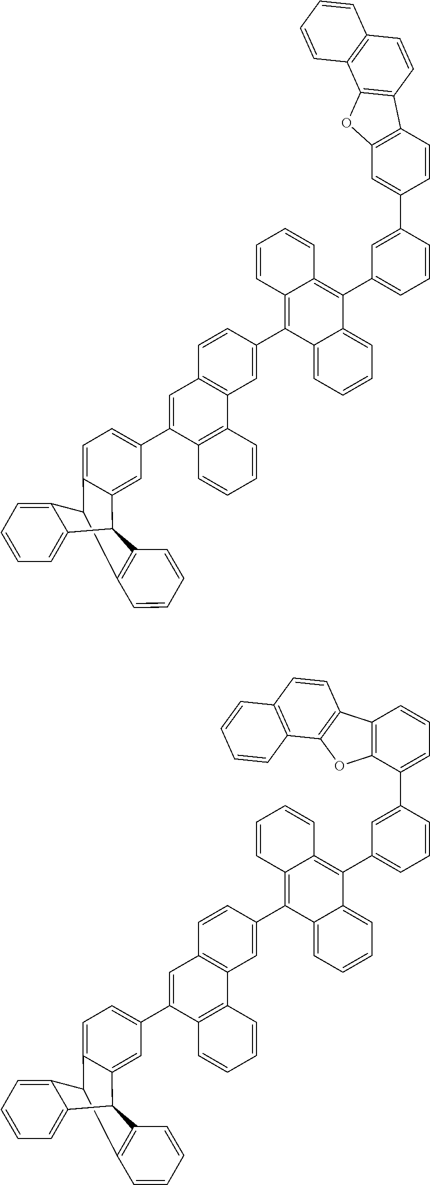

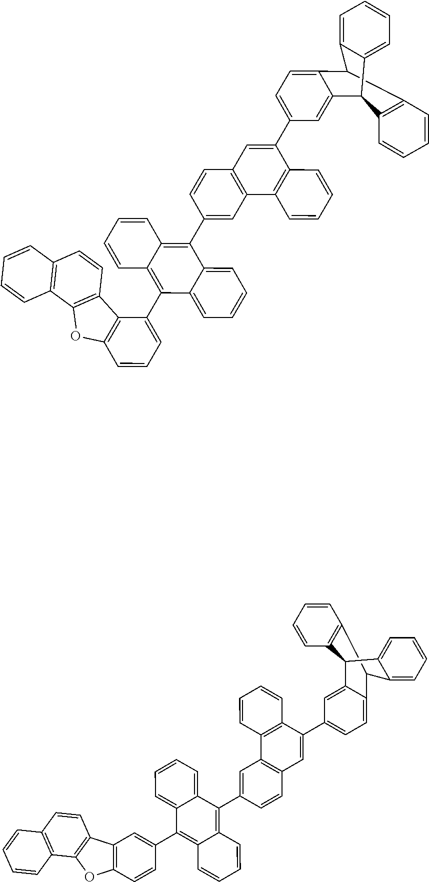

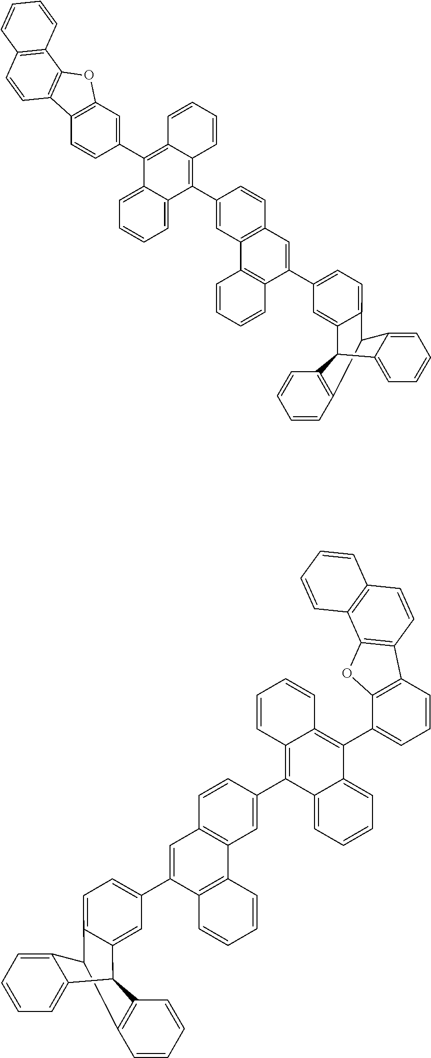

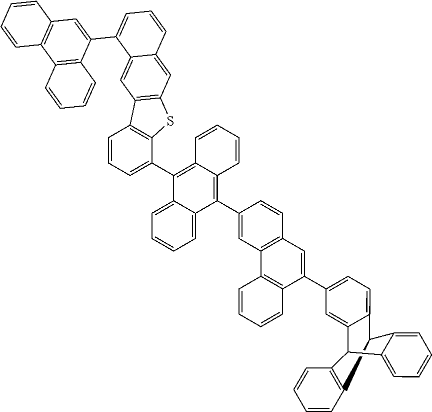

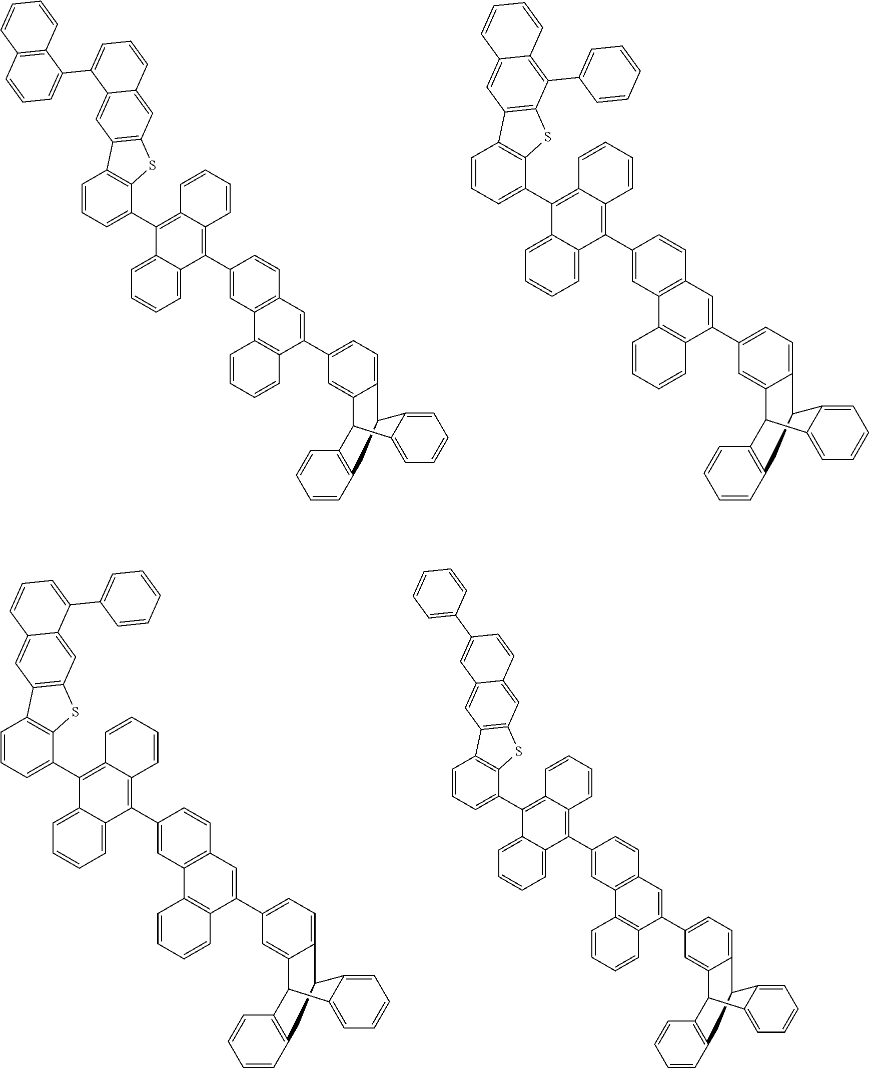

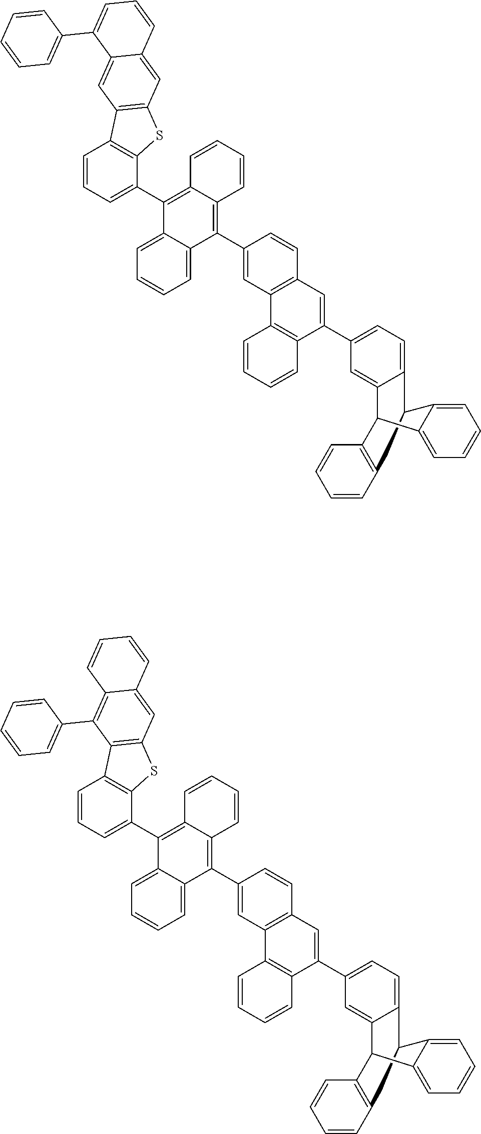

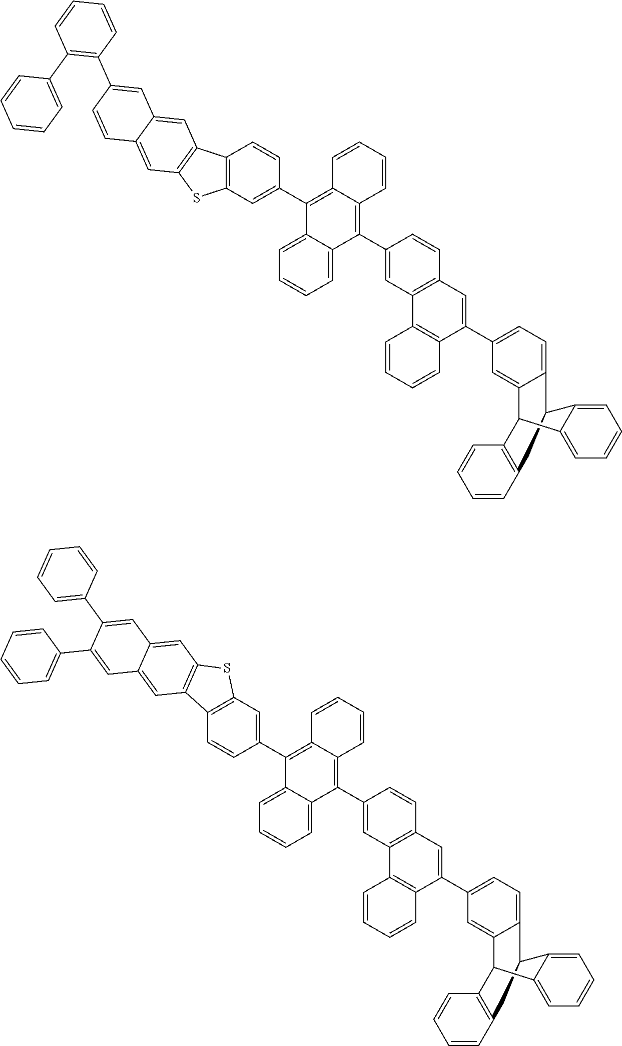

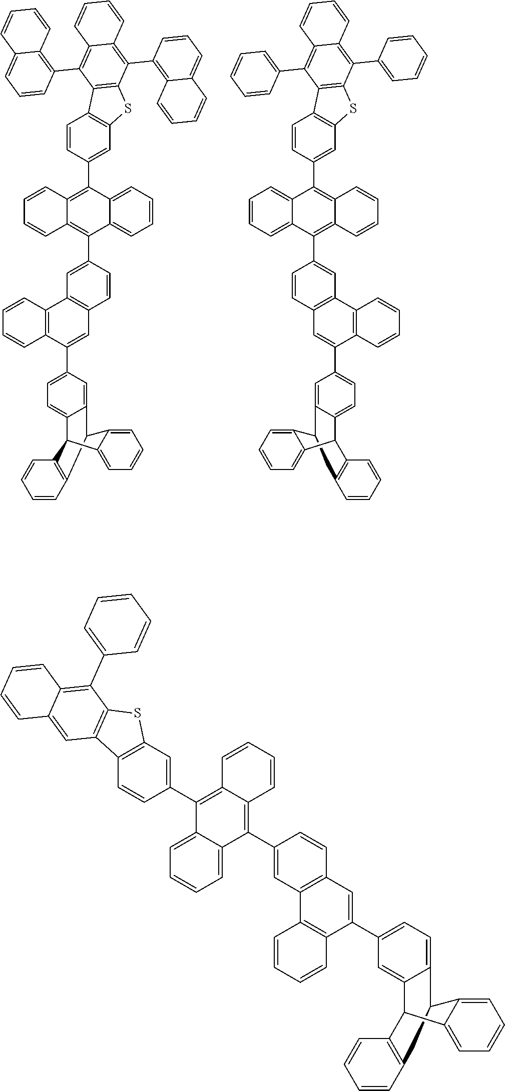

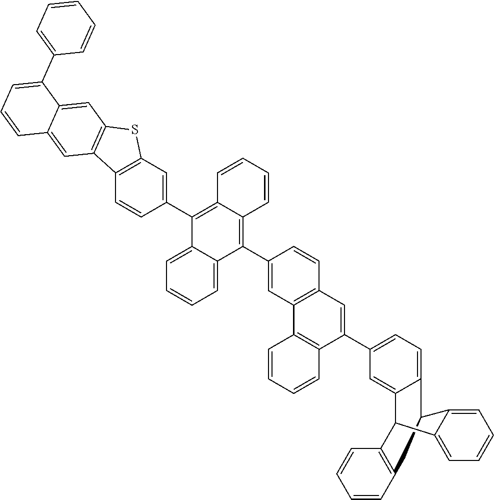

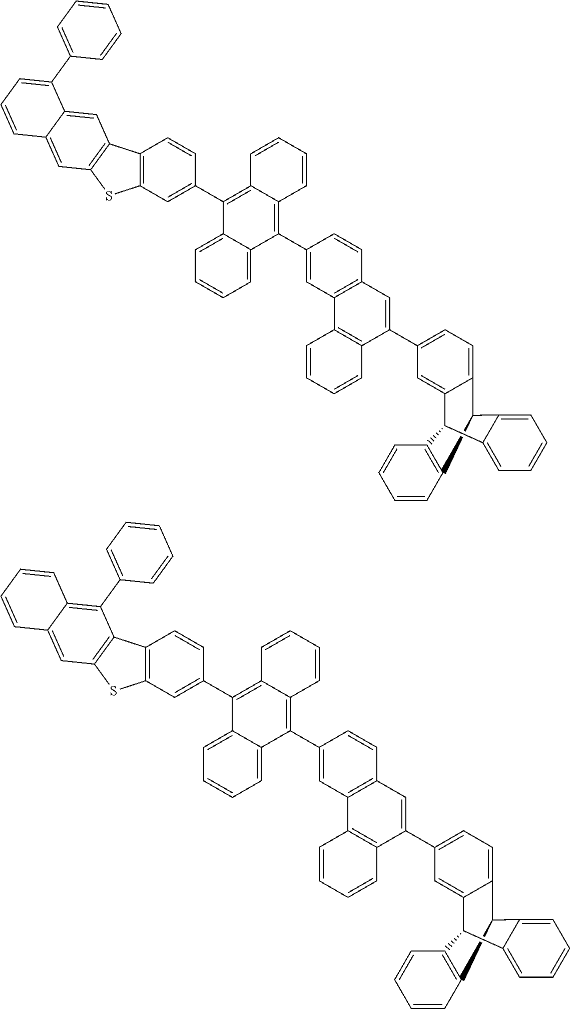 [SMILES: c1ccc(-c2cccc3cc4sc5cc(-c6c7ccccc7c(-c7ccc8cc(-c9ccc%10c(c9)[C@H]9c%11ccccc%11[C@@H]%10c%10ccccc%109)c9ccccc9c8c7)c7ccccc67)ccc5c4cc23)cc1 ; c1ccc(-c2c3ccccc3cc3sc4cc(-c5c6ccccc6c(-c6ccc7cc(-c8ccc9c(c8)[C@H]8c%10ccccc%10[C@@H]9c9ccccc98)c8ccccc8c7c6)c6ccccc56)ccc4c23)cc1]

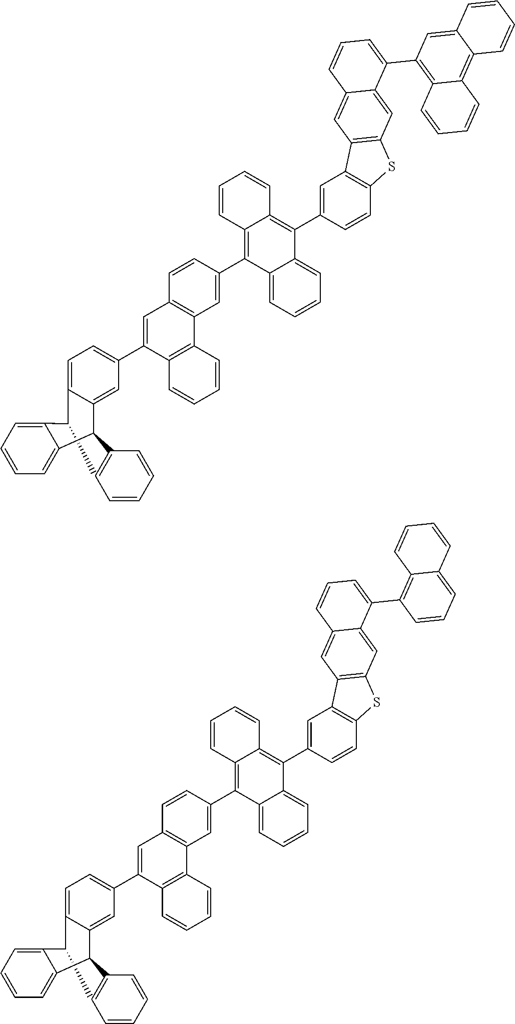 [SMILES: c1cc(-c2cc3ccccc3c3ccccc23)c2cc3sc4ccc(-c5c6ccccc6c(-c6ccc7cc(-c8ccc9c(c8)[C@H]8c%10ccccc%10[C@@H]9c9ccccc98)c8ccccc8c7c6)c6ccccc56)cc4c3cc2c1 ; c1ccc2c(-c3cccc4cc5c(cc34)sc3ccc(-c4c6ccccc6c(-c6ccc7cc(-c8ccc9c(c8)[C@H]8c%10ccccc%10[C@@H]9c9ccccc98)c8ccccc8c7c6)c6ccccc46)cc35)cccc2c1]

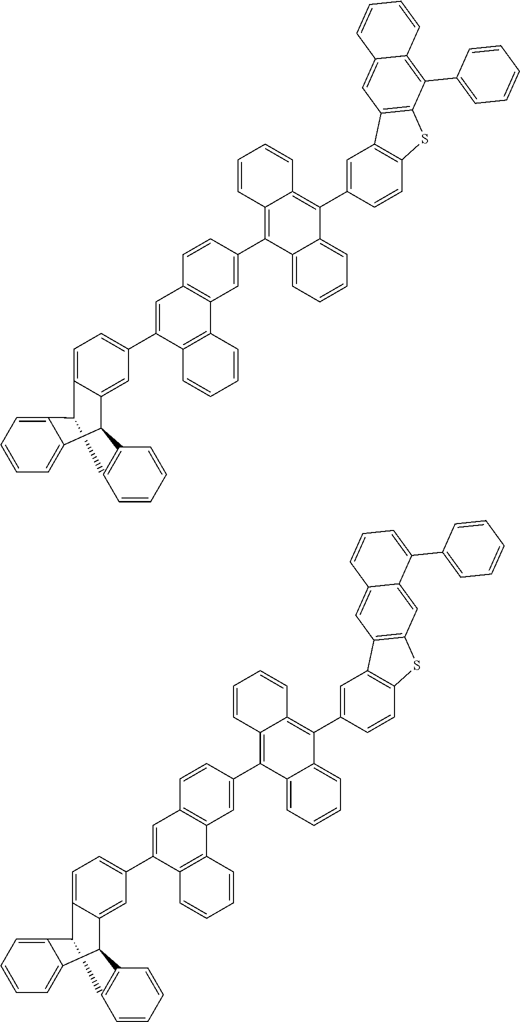 [SMILES: c1ccc(-c2c3ccccc3cc3c2sc2ccc(-c4c5ccccc5c(-c5ccc6cc(-c7ccc8c(c7)[C@H]7c9ccccc9[C@@H]8c8ccccc87)c7ccccc7c6c5)c5ccccc45)cc23)cc1 ; c1ccc(-c2cccc3cc4c(cc23)sc2ccc(-c3c5ccccc5c(-c5ccc6cc(-c7ccc8c(c7)[C@H]7c9ccccc9[C@@H]8c8ccccc87)c7ccccc7c6c5)c5ccccc35)cc24)cc1]

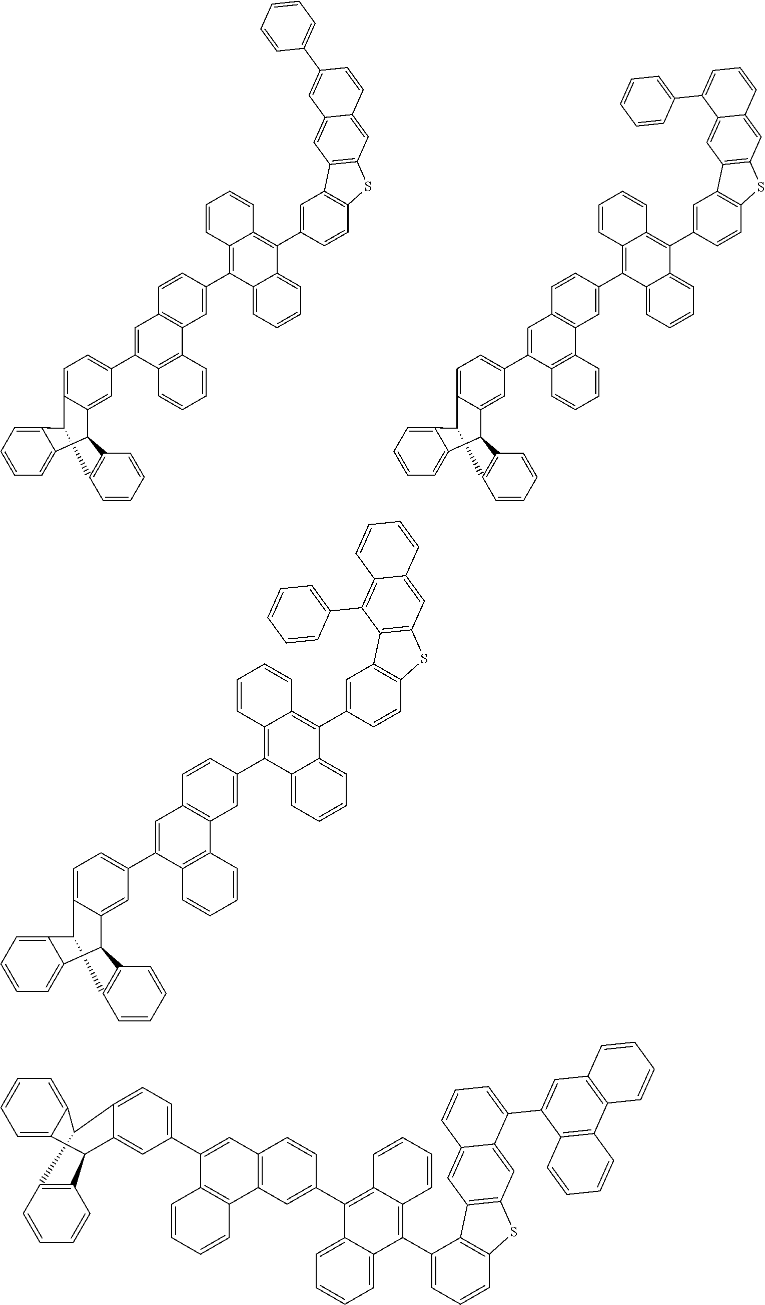

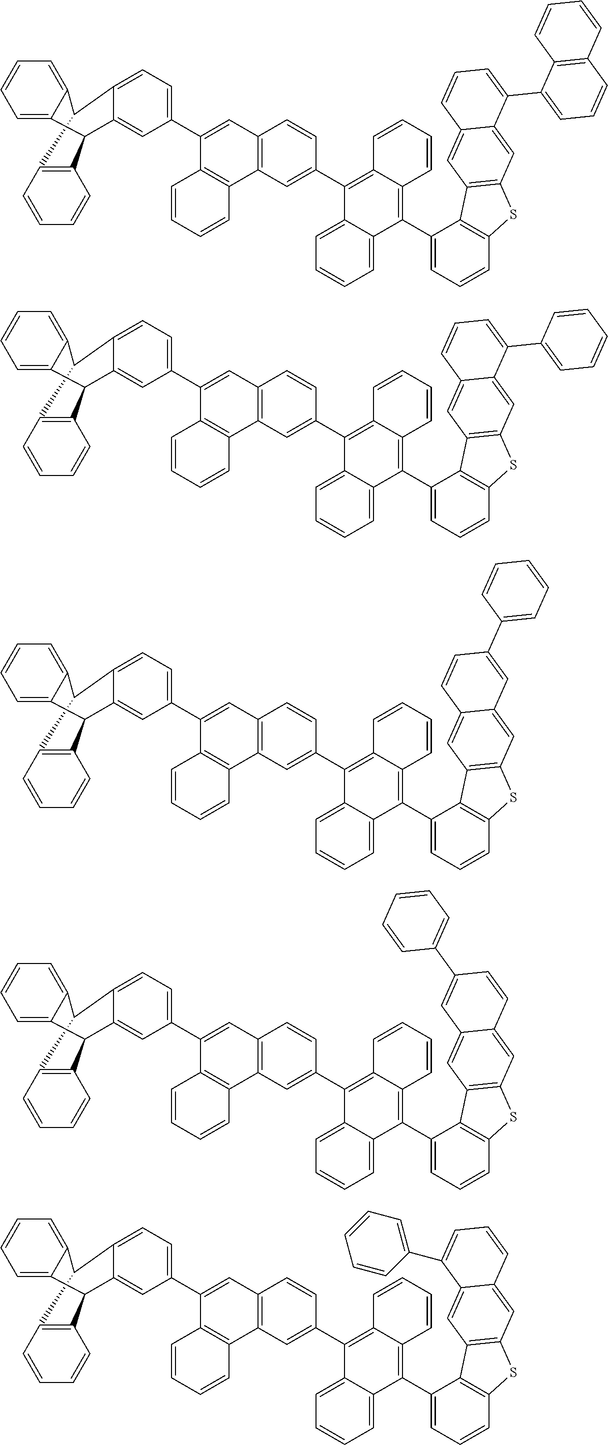

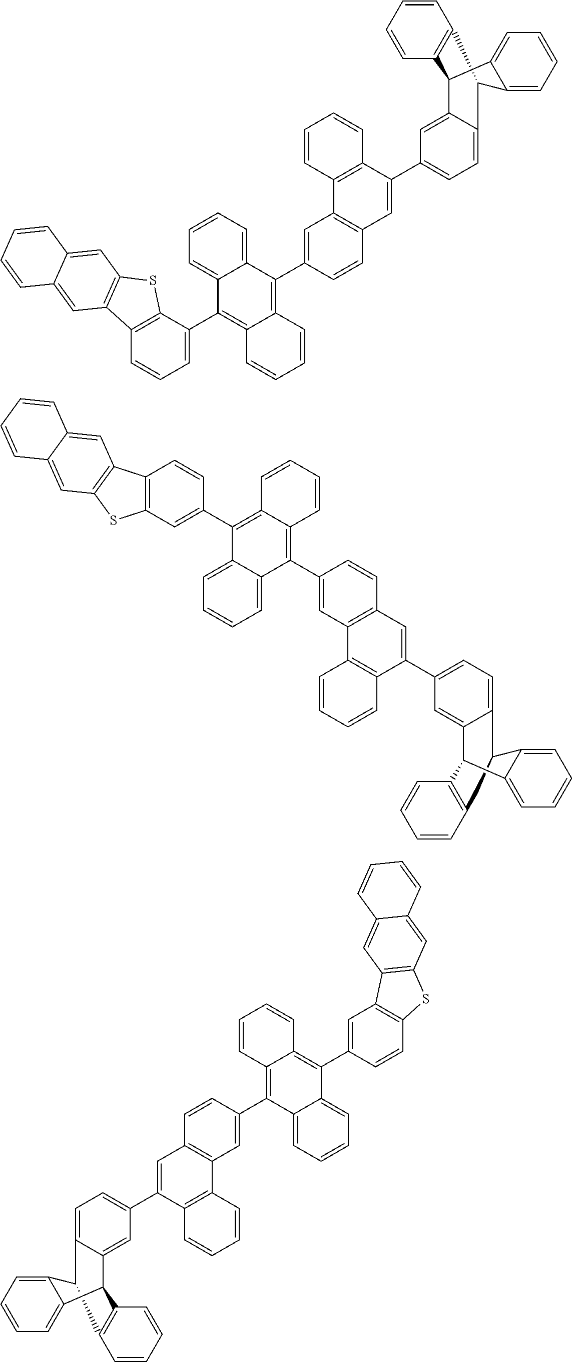

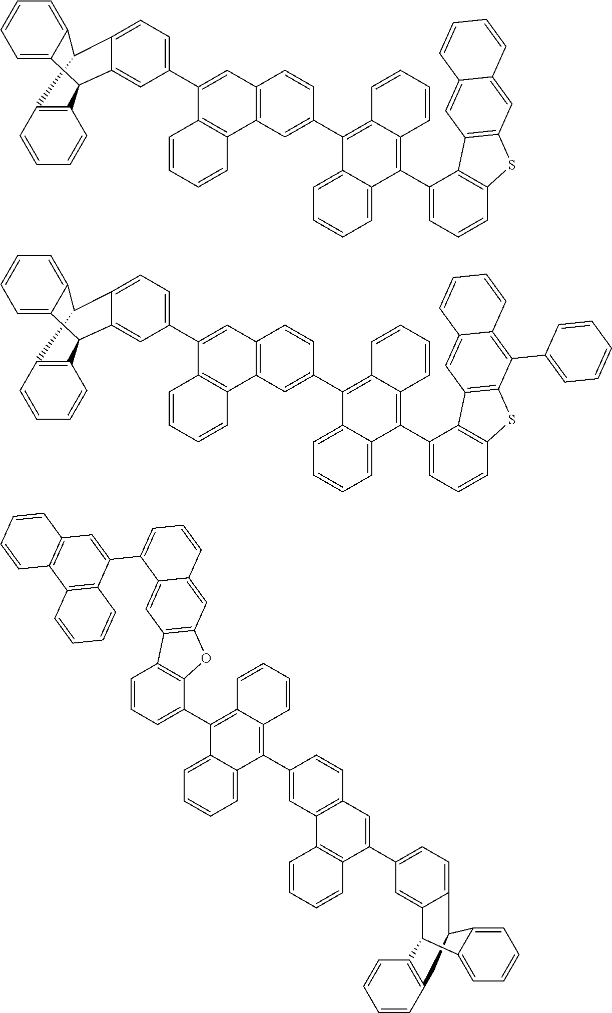

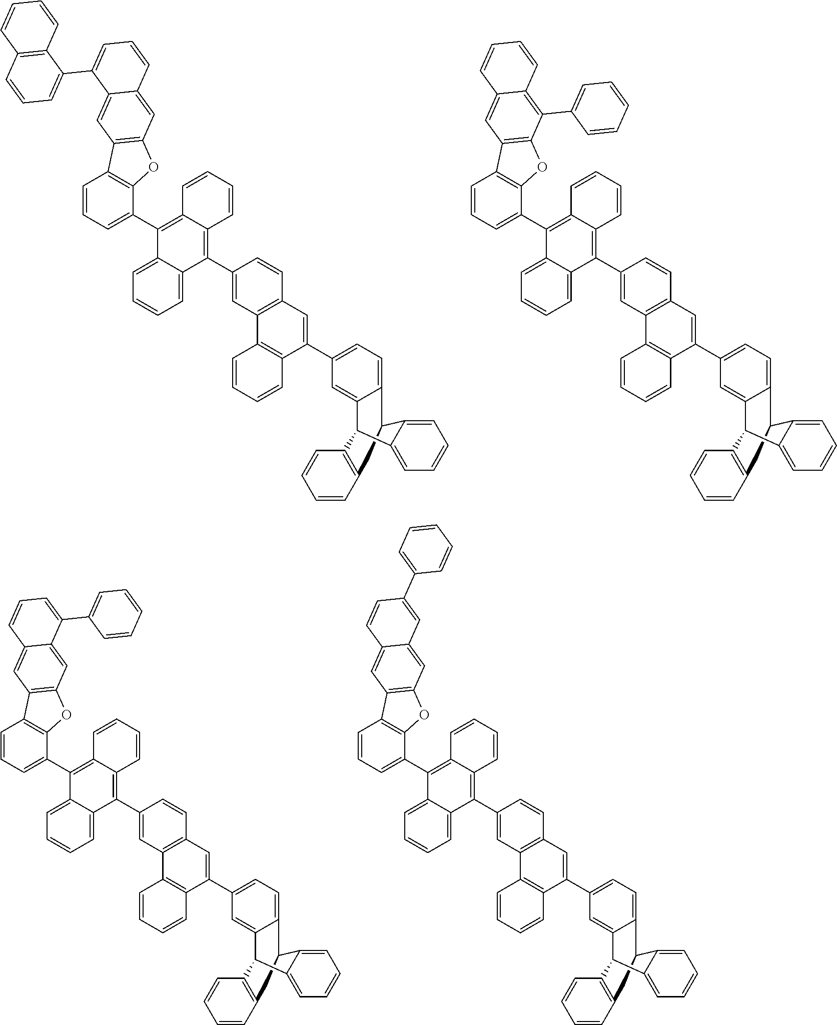

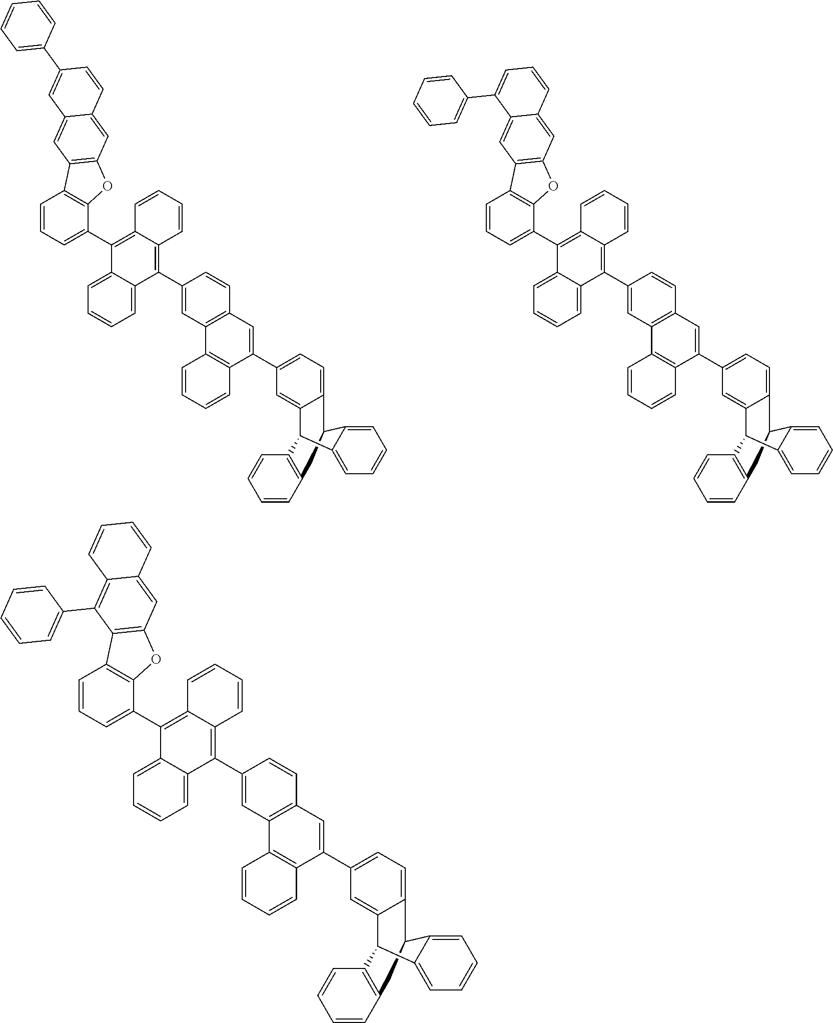 [SMILES: c1ccc(-c2ccc3cc4oc5c(-c6c7ccccc7c(-c7ccc8cc(-c9ccc%10c(c9)[C@H]9c%11ccccc%11[C@@H]%10c%10ccccc%109)c9ccccc9c8c7)c7ccccc67)cccc5c4cc3c2)cc1 ; c1ccc(-c2cccc3cc4oc5c(-c6c7ccccc7c(-c7ccc8cc(-c9ccc%10c(c9)[C@H]9c%11ccccc%11[C@@H]%10c%10ccccc%109)c9ccccc9c8c7)c7ccccc67)cccc5c4cc23)cc1 ; c1ccc(-c2c3ccccc3cc3oc4c(-c5c6ccccc6c(-c6ccc7cc(-c8ccc9c(c8)[C@H]8c%10ccccc%10[C@@H]9c9ccccc98)c8ccccc8c7c6)c6ccccc56)cccc4c23)cc1]

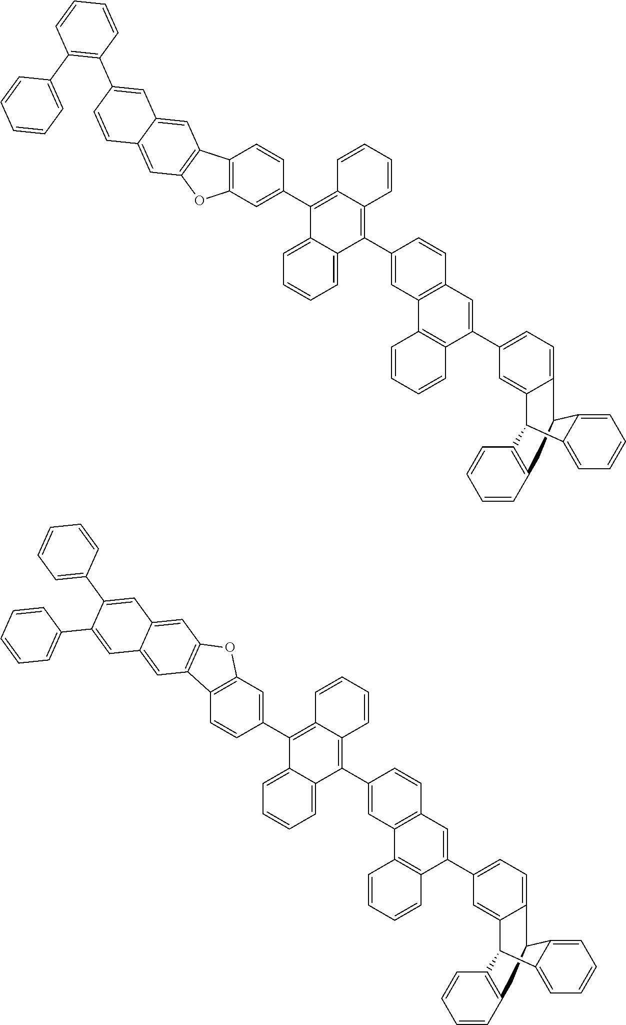 [SMILES: c1ccc(-c2ccccc2-c2ccc3cc4oc5cc(-c6c7ccccc7c(-c7ccc8cc(-c9ccc%10c(c9)[C@H]9c%11ccccc%11[C@@H]%10c%10ccccc%109)c9ccccc9c8c7)c7ccccc67)ccc5c4cc3c2)cc1 ; c1ccc(-c2cc3cc4oc5cc(-c6c7ccccc7c(-c7ccc8cc(-c9ccc%10c(c9)[C@H]9c%11ccccc%11[C@@H]%10c%10ccccc%109)c9ccccc9c8c7)c7ccccc67)ccc5c4cc3cc2-c2ccccc2)cc1]

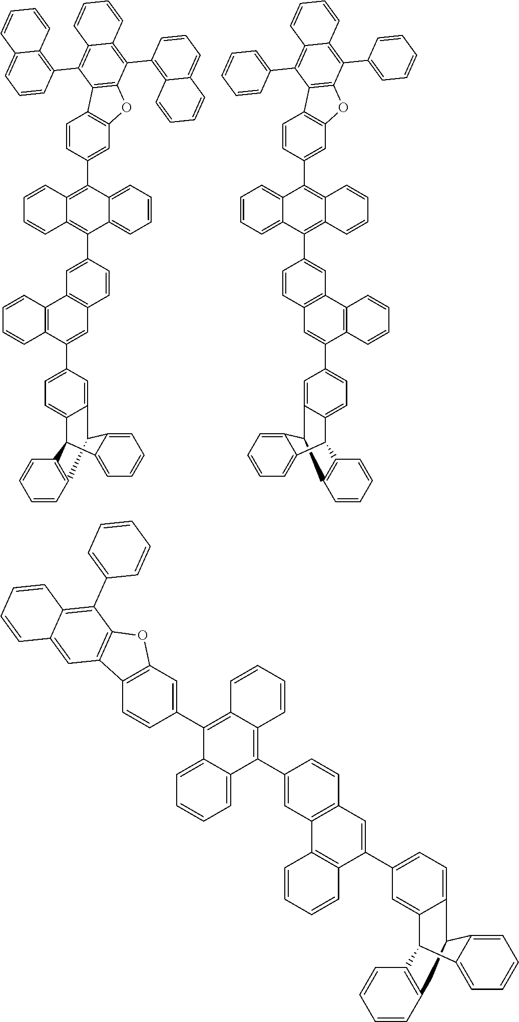

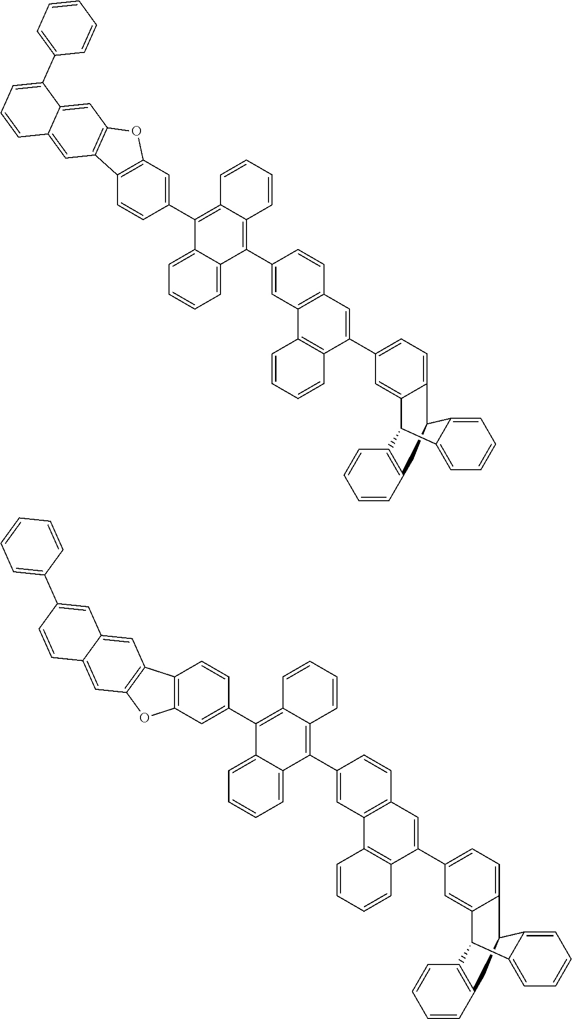 [SMILES: c1ccc(-c2cccc3cc4c(cc23)oc2cc(-c3c5ccccc5c(-c5ccc6cc(-c7ccc8c(c7)[C@H]7c9ccccc9[C@@H]8c8ccccc87)c7ccccc7c6c5)c5ccccc35)ccc24)cc1 ; c1ccc(-c2ccc3cc4oc5cc(-c6c7ccccc7c(-c7ccc8cc(-c9ccc%10c(c9)[C@H]9c%11ccccc%11[C@@H]%10c%10ccccc%109)c9ccccc9c8c7)c7ccccc67)ccc5c4cc3c2)cc1]

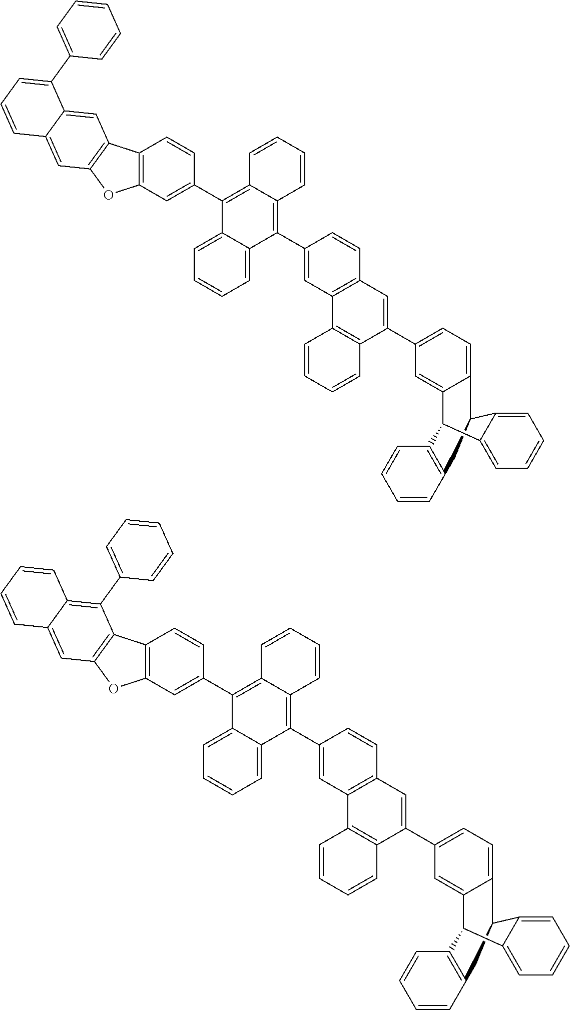 [SMILES: c1ccc(-c2cccc3cc4oc5cc(-c6c7ccccc7c(-c7ccc8cc(-c9ccc%10c(c9)[C@H]9c%11ccccc%11[C@@H]%10c%10ccccc%109)c9ccccc9c8c7)c7ccccc67)ccc5c4cc23)cc1 ; c1ccc(-c2c3ccccc3cc3oc4cc(-c5c6ccccc6c(-c6ccc7cc(-c8ccc9c(c8)[C@H]8c%10ccccc%10[C@@H]9c9ccccc98)c8ccccc8c7c6)c6ccccc56)ccc4c23)cc1]

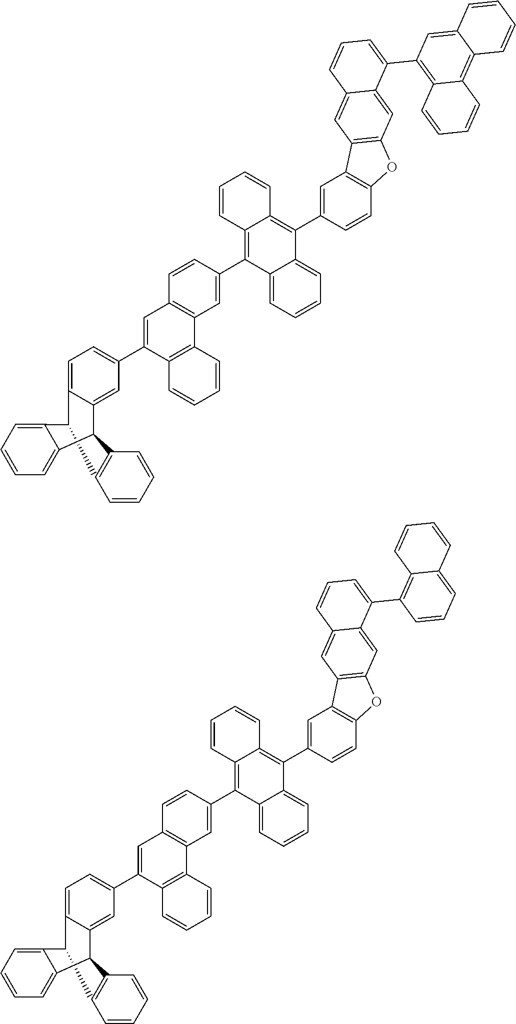 [SMILES: c1cc(-c2cc3ccccc3c3ccccc23)c2cc3oc4ccc(-c5c6ccccc6c(-c6ccc7cc(-c8ccc9c(c8)[C@H]8c%10ccccc%10[C@@H]9c9ccccc98)c8ccccc8c7c6)c6ccccc56)cc4c3cc2c1 ; c1ccc2c(-c3cccc4cc5c(cc34)oc3ccc(-c4c6ccccc6c(-c6ccc7cc(-c8ccc9c(c8)[C@H]8c%10ccccc%10[C@@H]9c9ccccc98)c8ccccc8c7c6)c6ccccc46)cc35)cccc2c1]

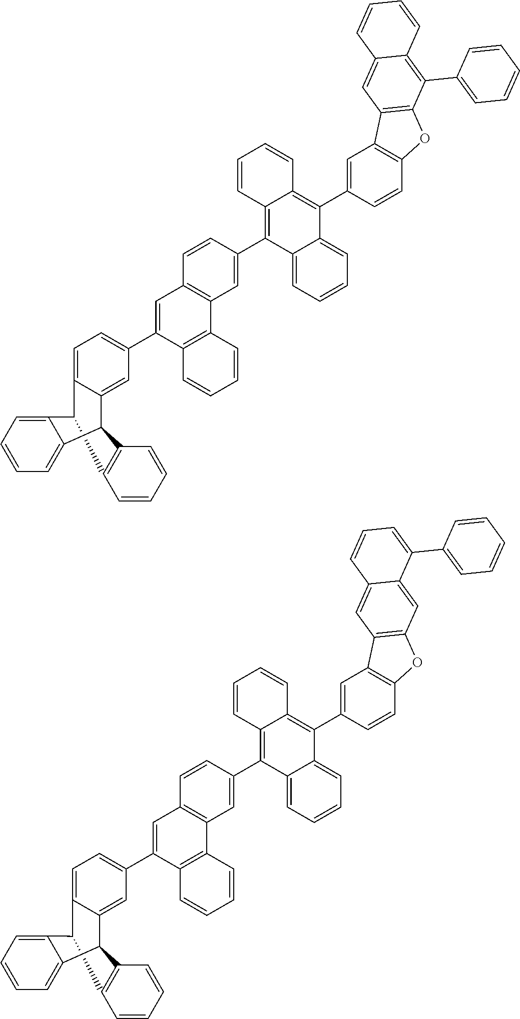 [SMILES: c1ccc(-c2c3ccccc3cc3c2oc2ccc(-c4c5ccccc5c(-c5ccc6cc(-c7ccc8c(c7)[C@H]7c9ccccc9[C@@H]8c8ccccc87)c7ccccc7c6c5)c5ccccc45)cc23)cc1 ; c1ccc(-c2cccc3cc4c(cc23)oc2ccc(-c3c5ccccc5c(-c5ccc6cc(-c7ccc8c(c7)[C@H]7c9ccccc9[C@@H]8c8ccccc87)c7ccccc7c6c5)c5ccccc35)cc24)cc1]

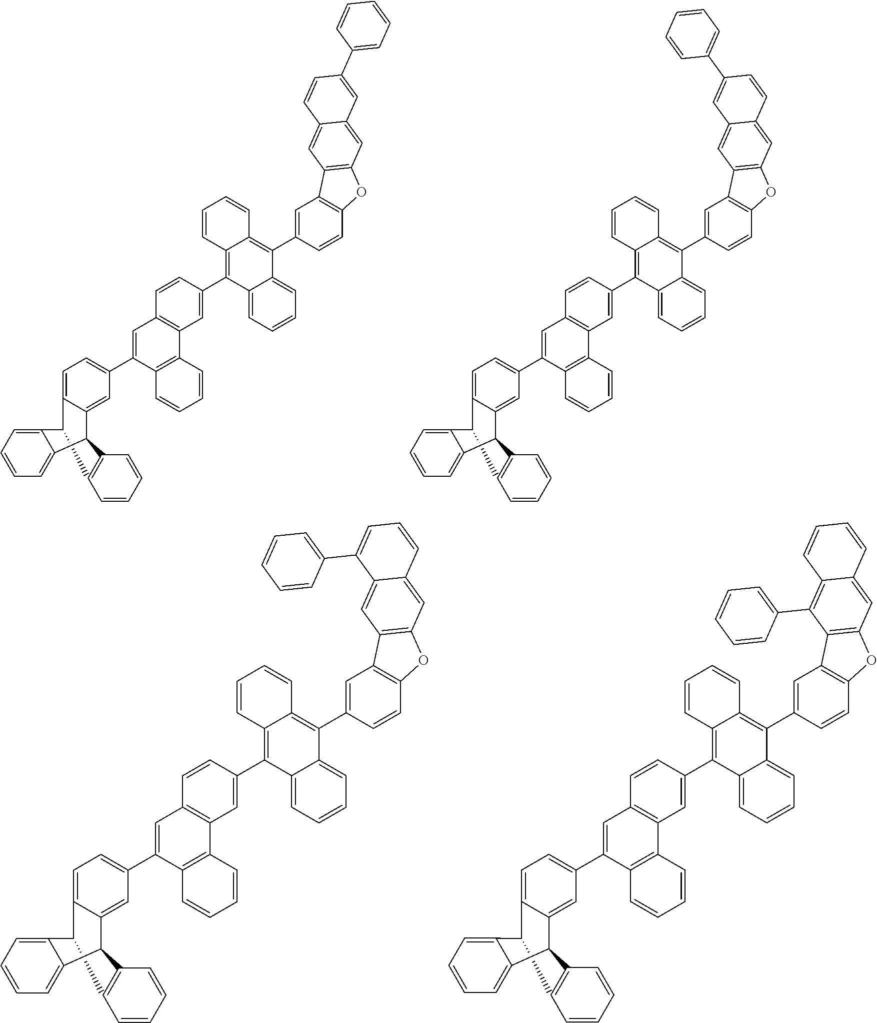

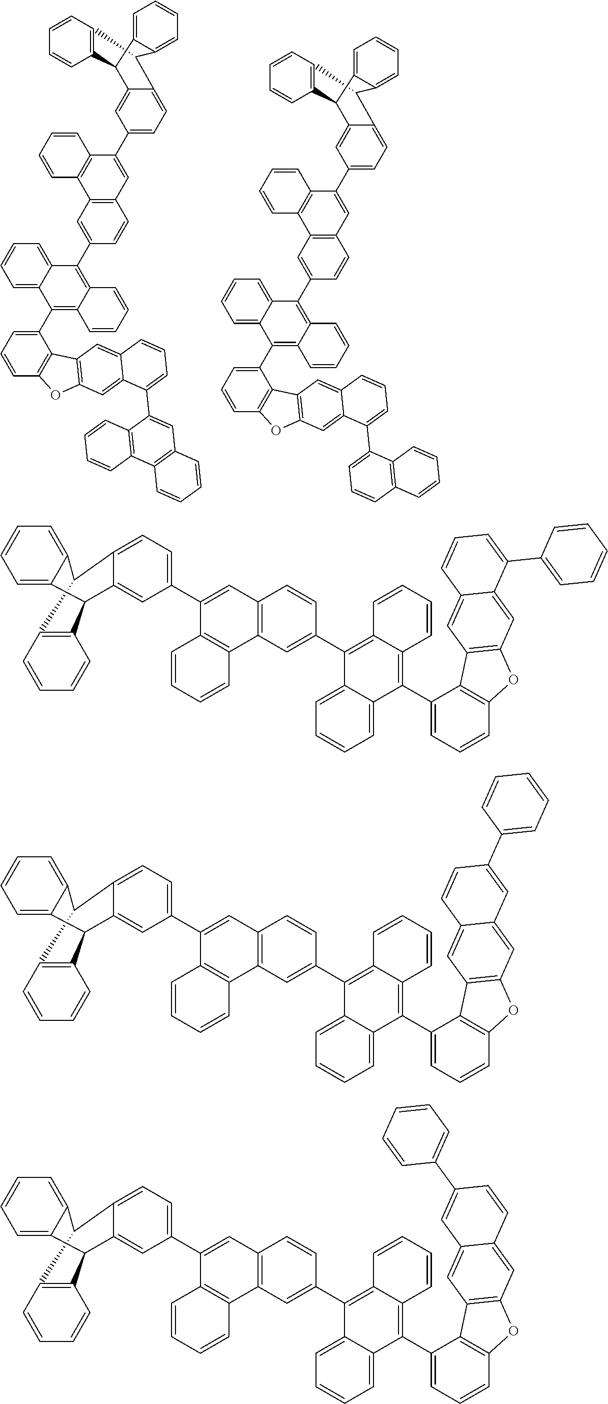

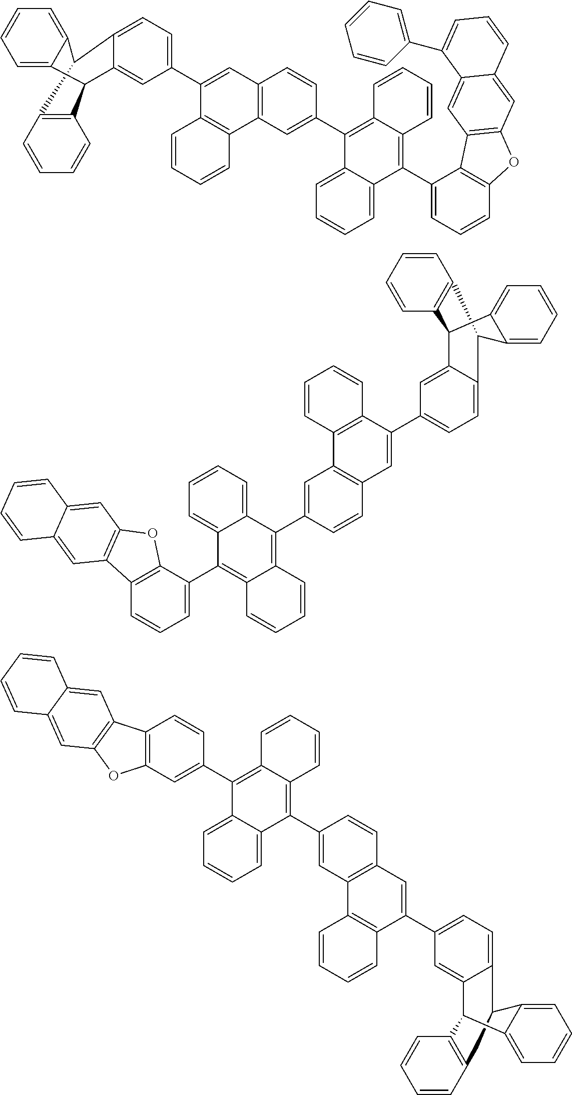

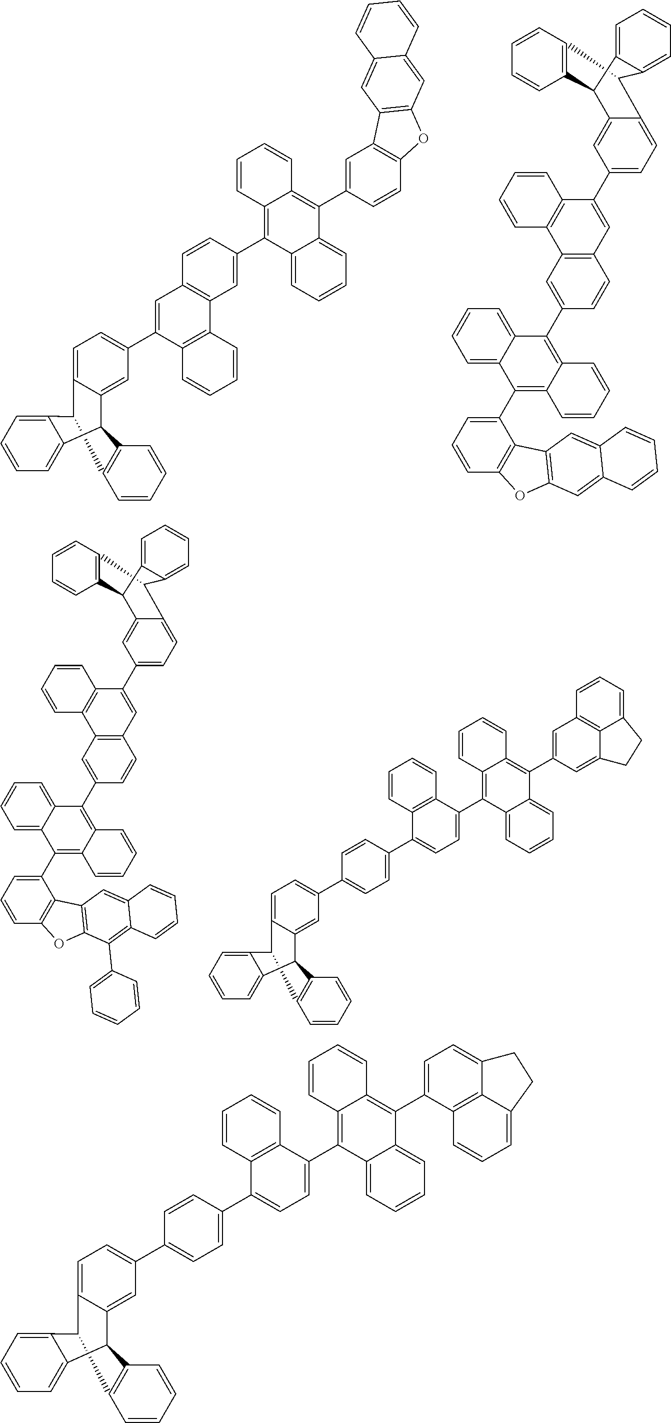

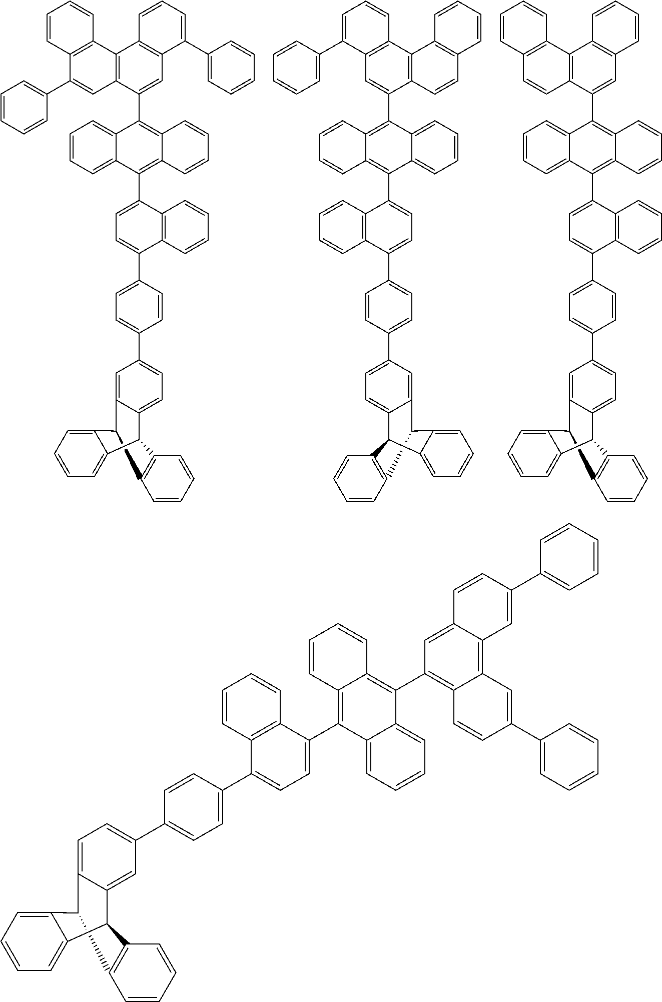

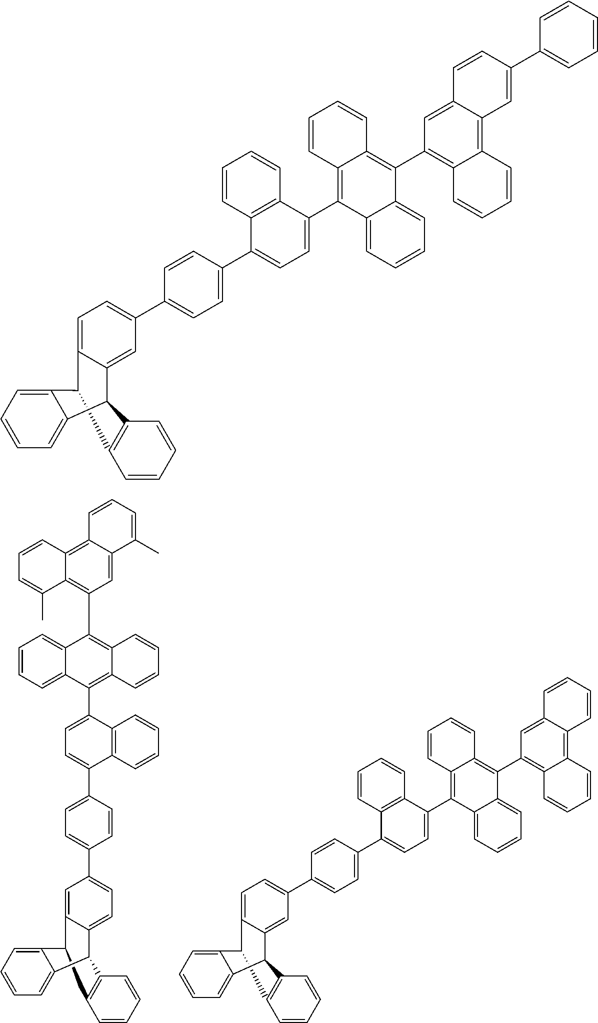

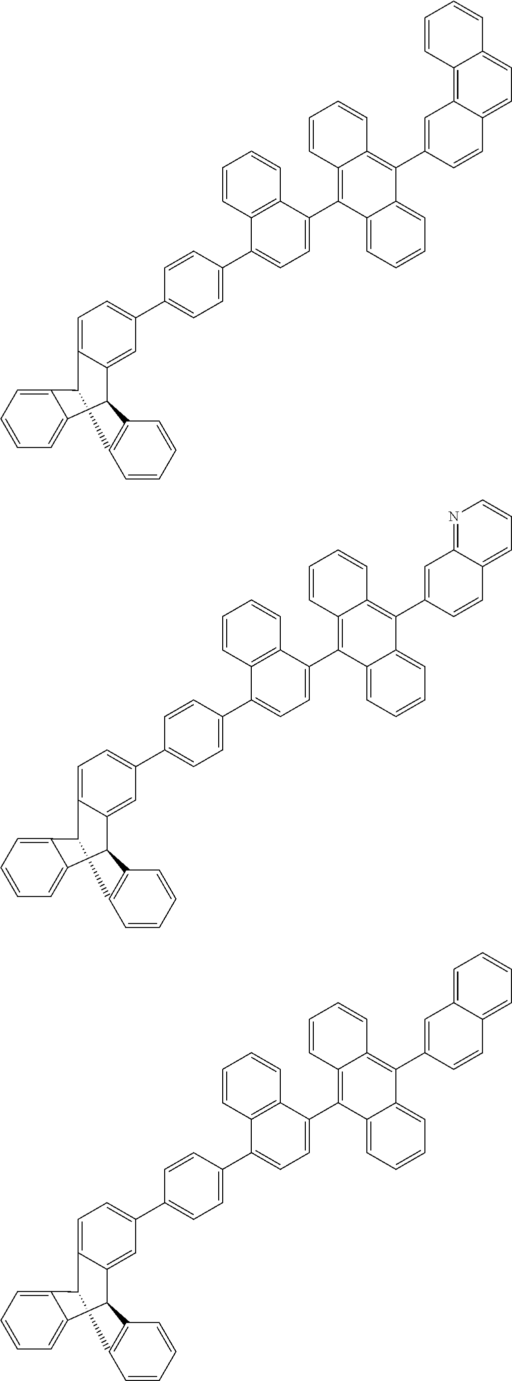

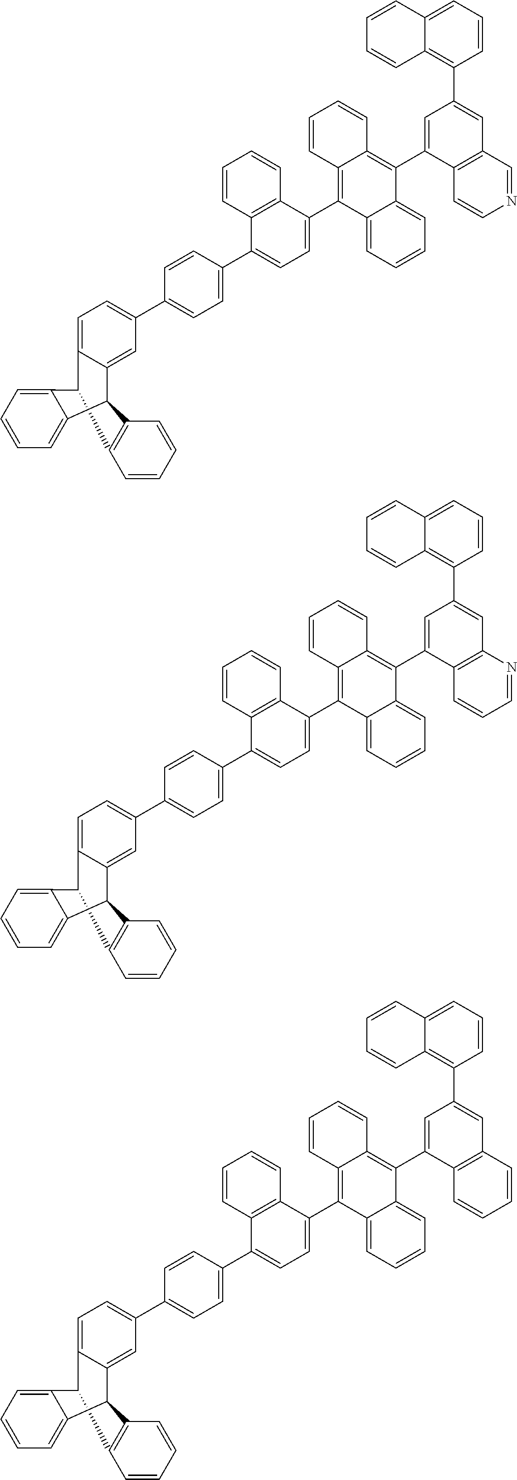

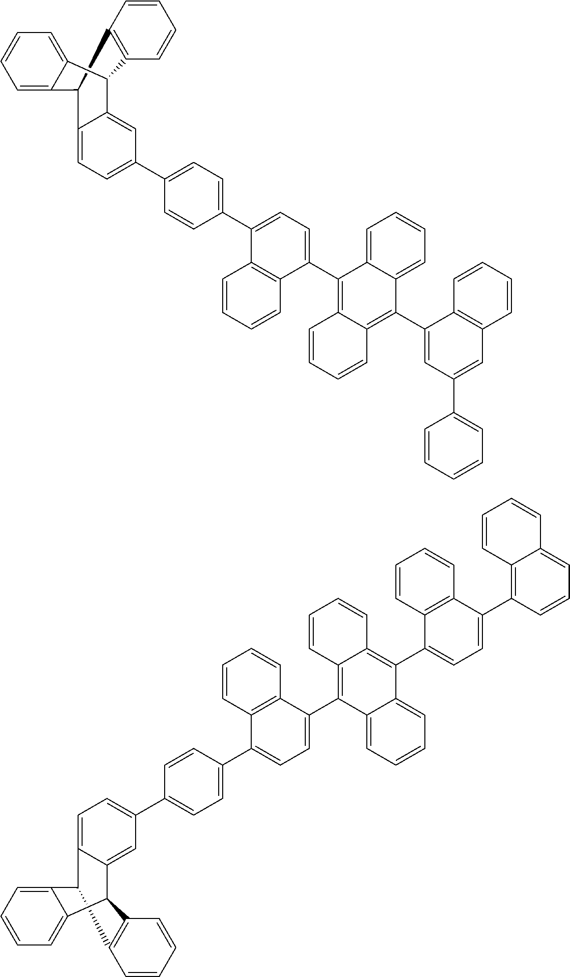

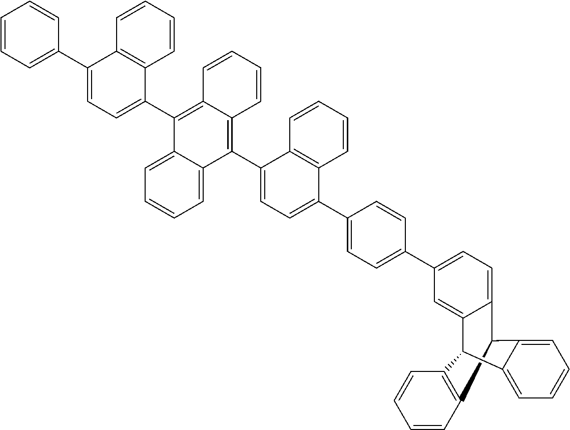 [SMILES: c1ccc(-c2ccc(-c3c4ccccc4c(-c4ccc(-c5ccc(-c6ccc7c(c6)[C@H]6c8ccccc8[C@@H]7c7ccccc76)cc5)c5ccccc45)c4ccccc34)c3ccccc23)cc1]

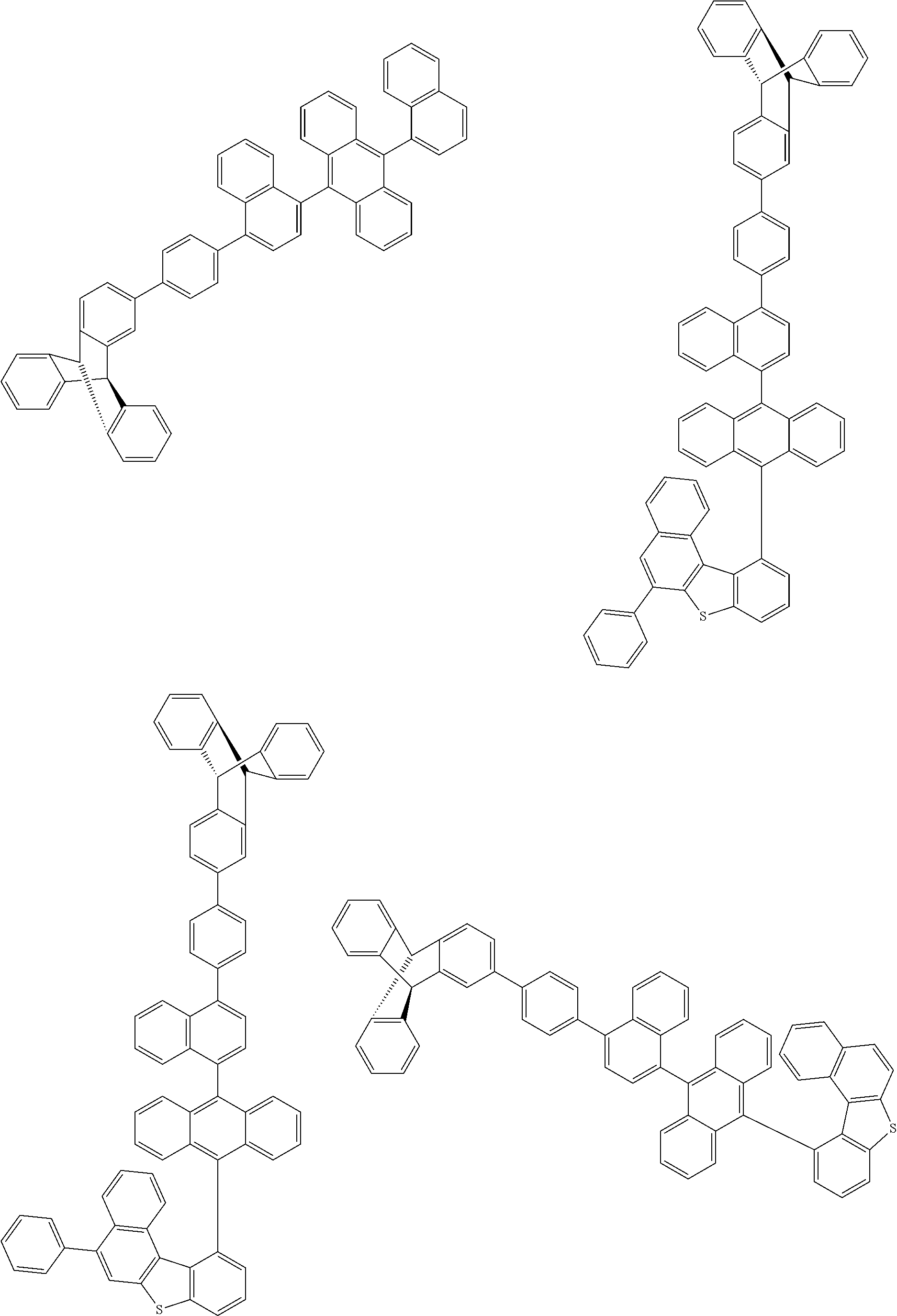

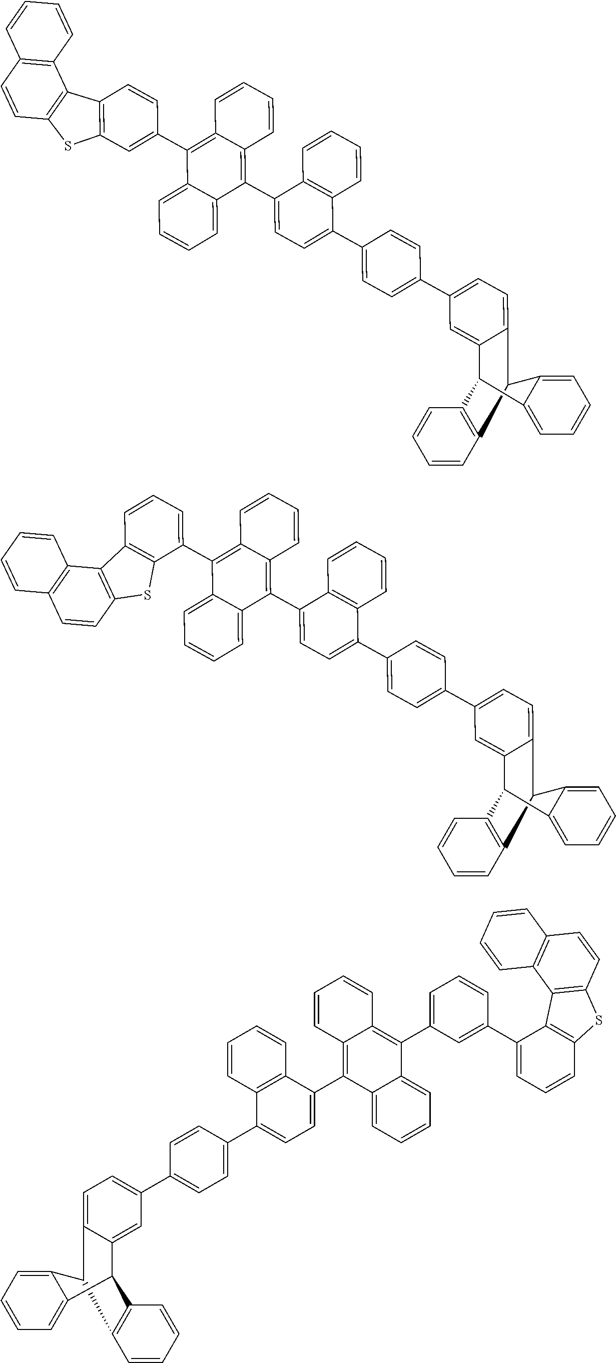

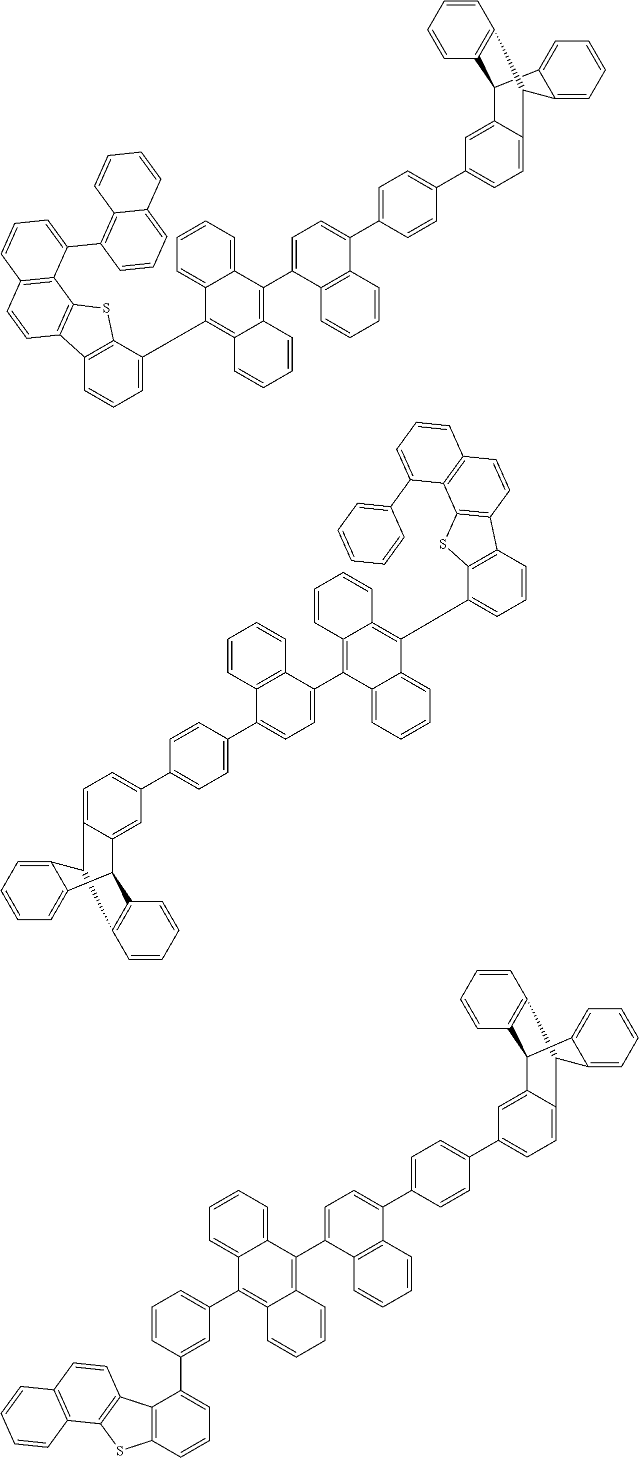

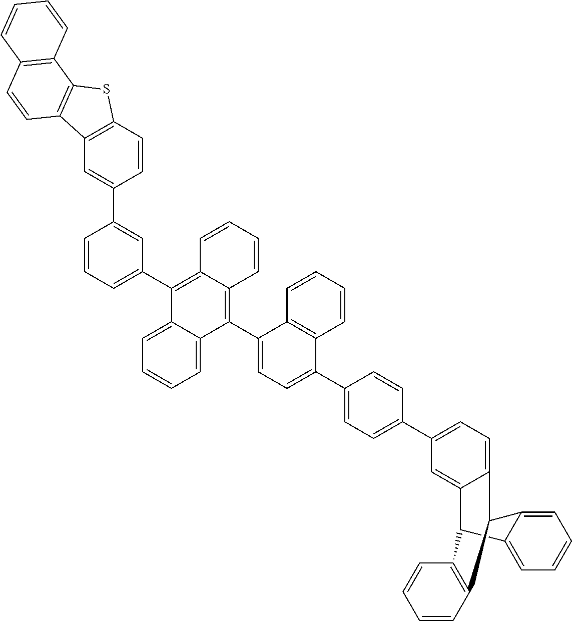

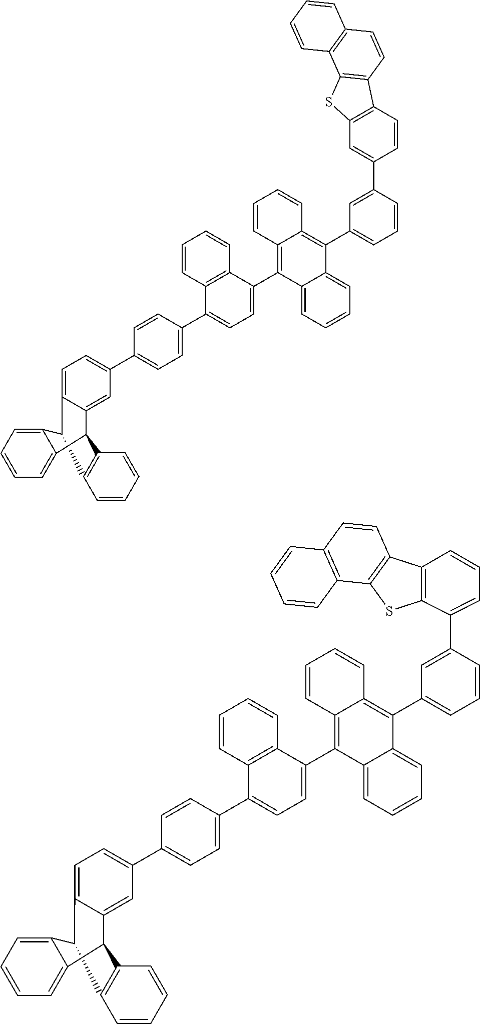 [SMILES: c1cc(-c2ccc3c(c2)sc2c4ccccc4ccc32)cc(-c2c3ccccc3c(-c3ccc(-c4ccc(-c5ccc6c(c5)[C@H]5c7ccccc7[C@@H]6c6ccccc65)cc4)c4ccccc34)c3ccccc23)c1 ; c1cc(-c2c3ccccc3c(-c3ccc(-c4ccc(-c5ccc6c(c5)[C@H]5c7ccccc7[C@@H]6c6ccccc65)cc4)c4ccccc34)c3ccccc23)cc(-c2cccc3c2sc2c4ccccc4ccc32)c1]

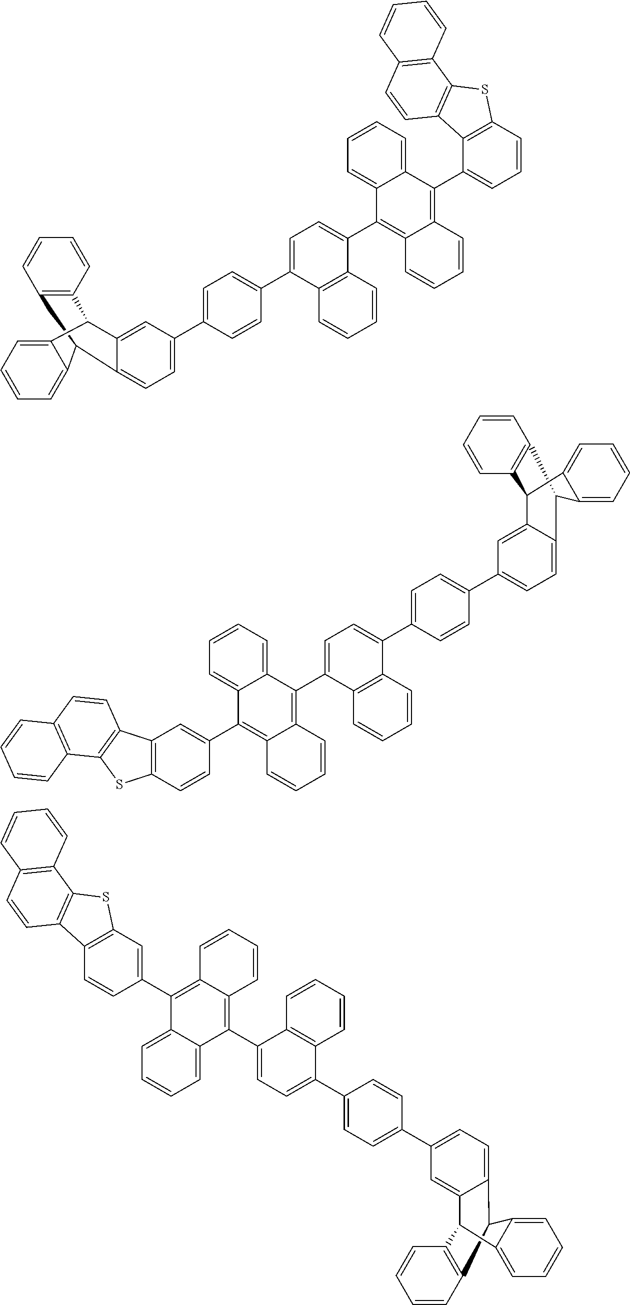

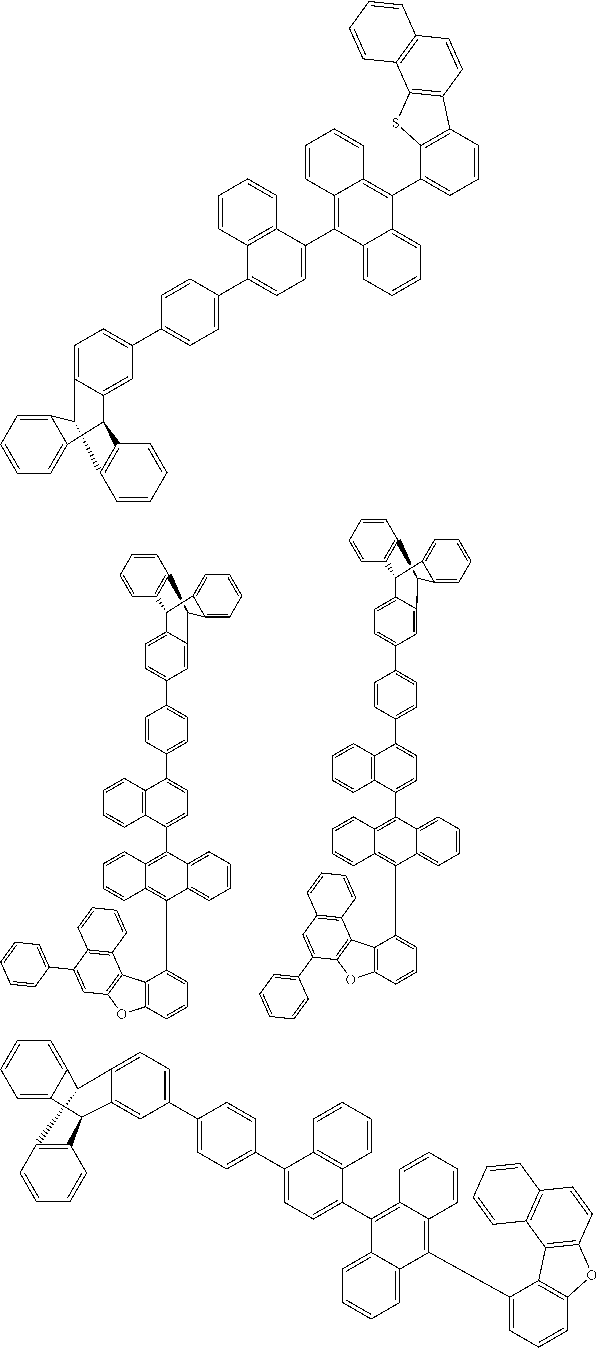

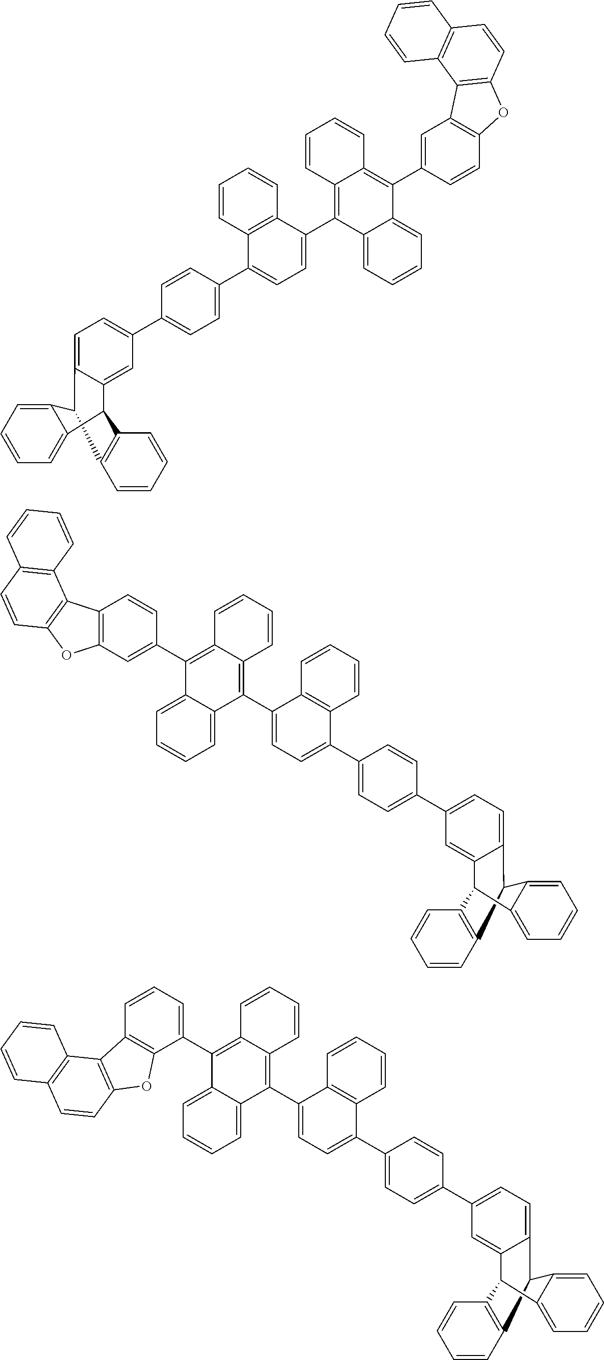 [SMILES: c1ccc2c(c1)ccc1oc3ccc(-c4c5ccccc5c(-c5ccc(-c6ccc(-c7ccc8c(c7)[C@H]7c9ccccc9[C@@H]8c8ccccc87)cc6)c6ccccc56)c5ccccc45)cc3c12 ; c1ccc2c(c1)ccc1oc3cc(-c4c5ccccc5c(-c5ccc(-c6ccc(-c7ccc8c(c7)[C@H]7c9ccccc9[C@@H]8c8ccccc87)cc6)c6ccccc56)c5ccccc45)ccc3c12 ; c1ccc2c(c1)ccc1oc3c(-c4c5ccccc5c(-c5ccc(-c6ccc(-c7ccc8c(c7)[C@H]7c9ccccc9[C@@H]8c8ccccc87)cc6)c6ccccc56)c5ccccc45)cccc3c12]

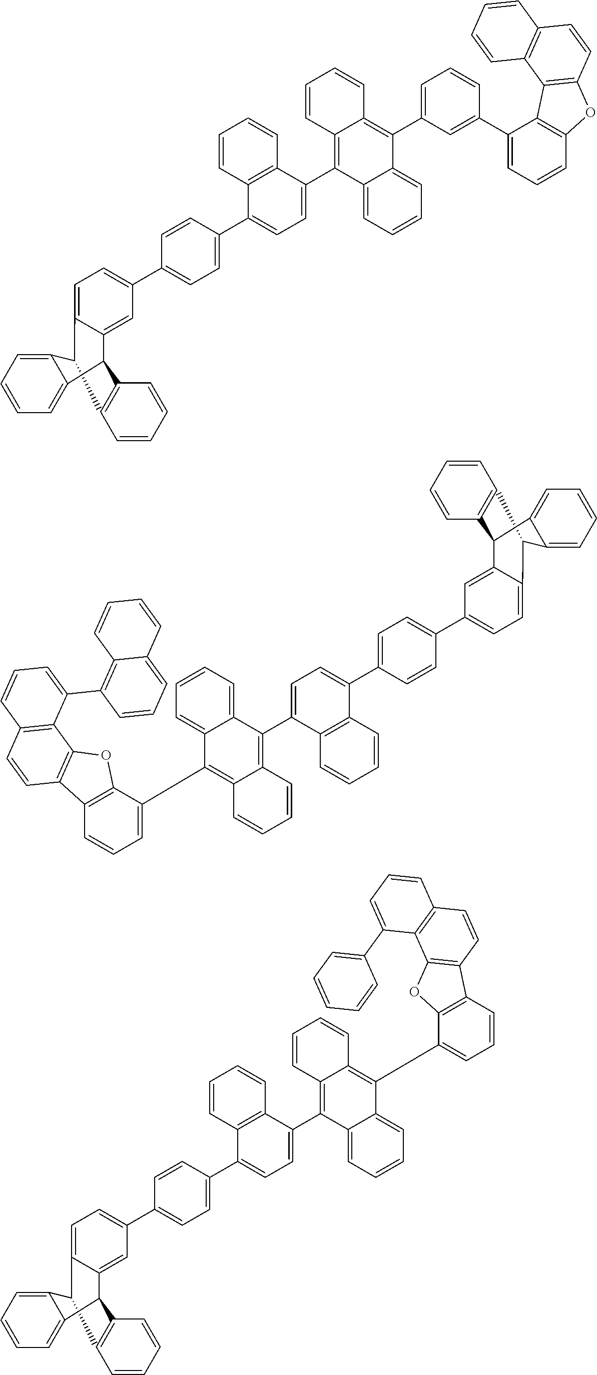 [SMILES: c1cc(-c2c3ccccc3c(-c3ccc(-c4ccc(-c5ccc6c(c5)[C@H]5c7ccccc7[C@@H]6c6ccccc65)cc4)c4ccccc34)c3ccccc23)cc(-c2cccc3oc4ccc5ccccc5c4c23)c1 ; c1ccc2c(-c3cccc4ccc5c6cccc(-c7c8ccccc8c(-c8ccc(-c9ccc(-c%10ccc%11c(c%10)[C@H]%10c%12ccccc%12[C@@H]%11c%11ccccc%11%10)cc9)c9ccccc89)c8ccccc78)c6oc5c34)cccc2c1 ; c1ccc(-c2cccc3ccc4c5cccc(-c6c7ccccc7c(-c7ccc(-c8ccc(-c9ccc%10c(c9)[C@H]9c%11ccccc%11[C@@H]%10c%10ccccc%109)cc8)c8ccccc78)c7ccccc67)c5oc4c23)cc1]

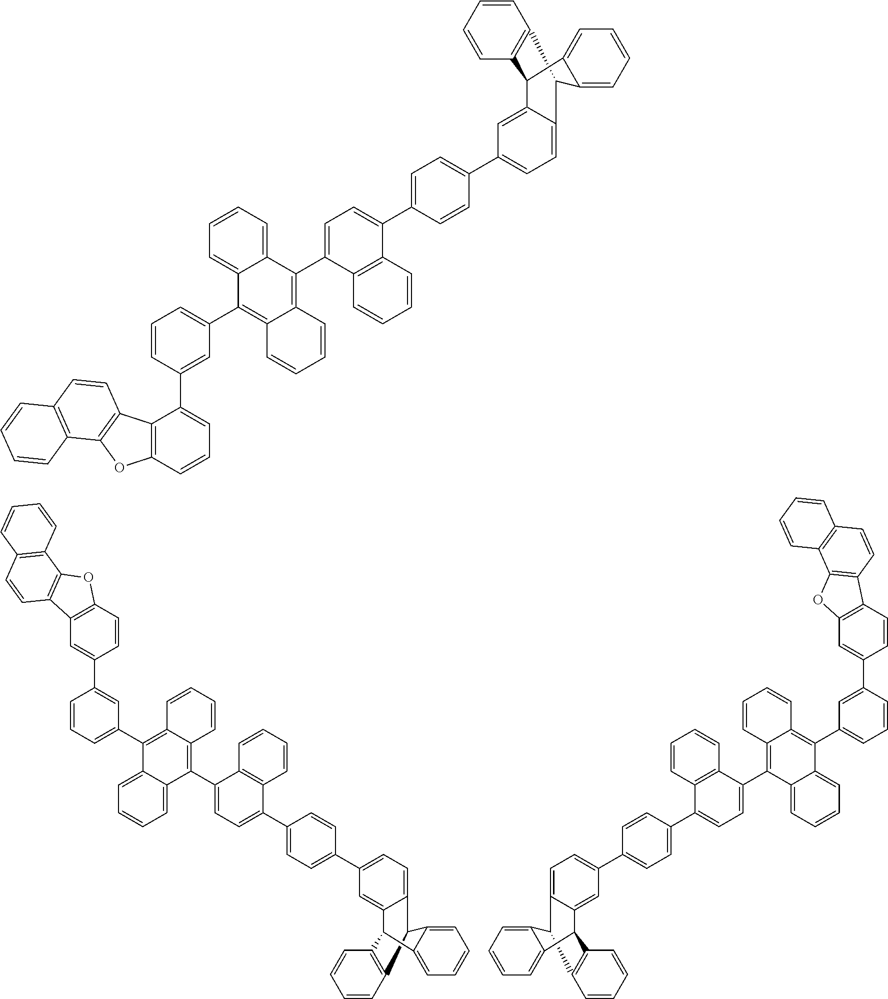

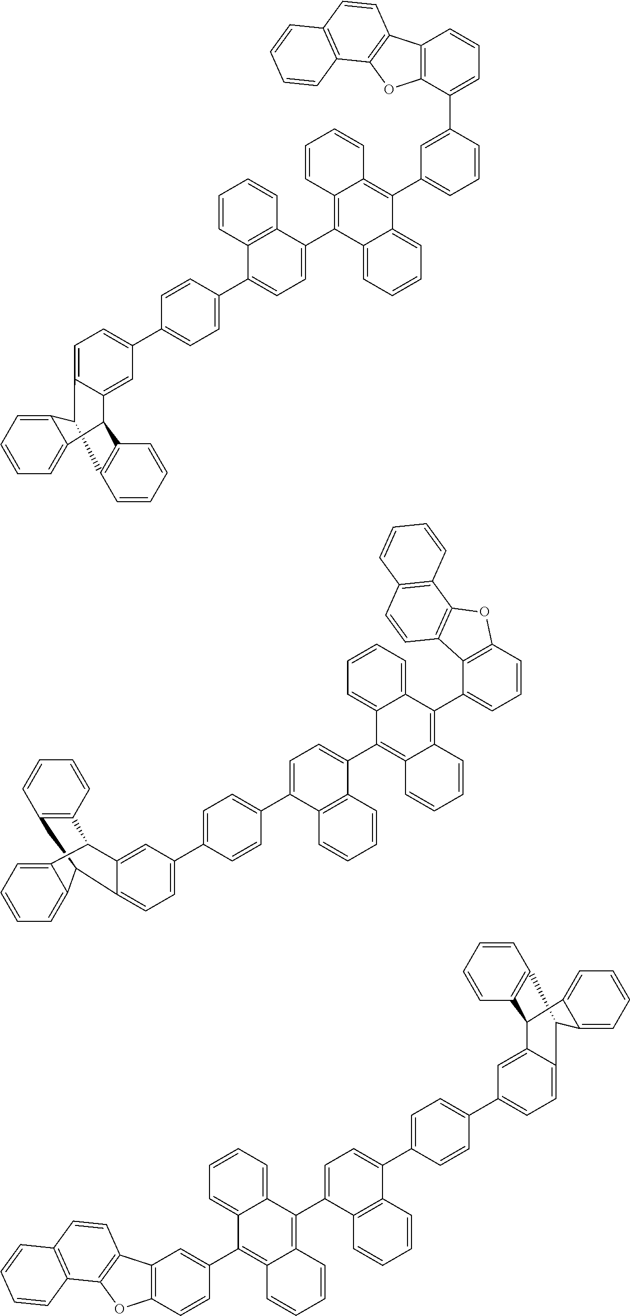 [SMILES: c1cc(-c2c3ccccc3c(-c3ccc(-c4ccc(-c5ccc6c(c5)[C@H]5c7ccccc7[C@@H]6c6ccccc65)cc4)c4ccccc34)c3ccccc23)cc(-c2cccc3c2oc2c4ccccc4ccc32)c1 ; c1ccc2c(c1)ccc1c2oc2cccc(-c3c4ccccc4c(-c4ccc(-c5ccc(-c6ccc7c(c6)[C@H]6c8ccccc8[C@@H]7c7ccccc76)cc5)c5ccccc45)c4ccccc34)c21 ; c1ccc2c(c1)ccc1c3cc(-c4c5ccccc5c(-c5ccc(-c6ccc(-c7ccc8c(c7)[C@H]7c9ccccc9[C@@H]8c8ccccc87)cc6)c6ccccc56)c5ccccc45)ccc3oc21]

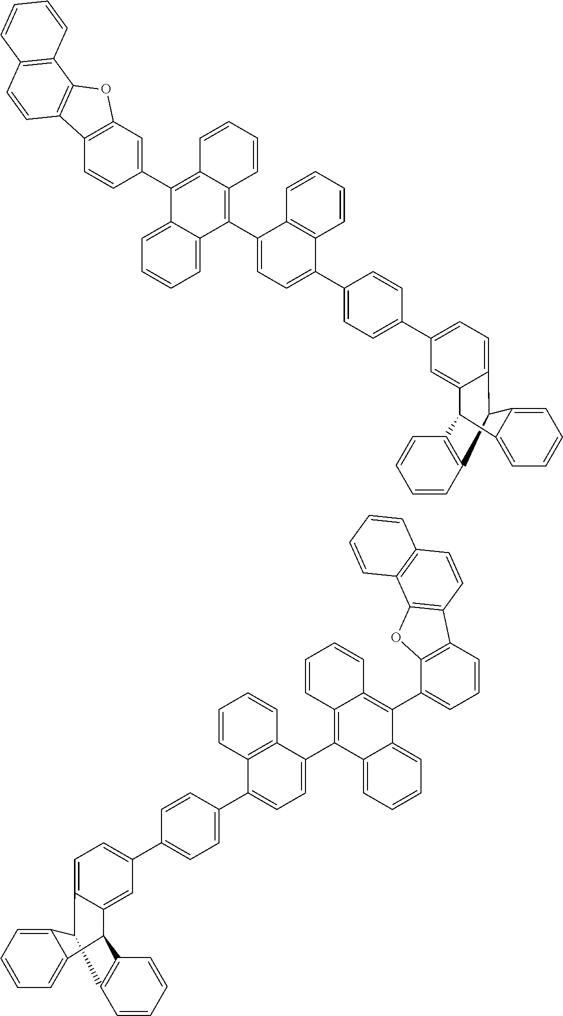

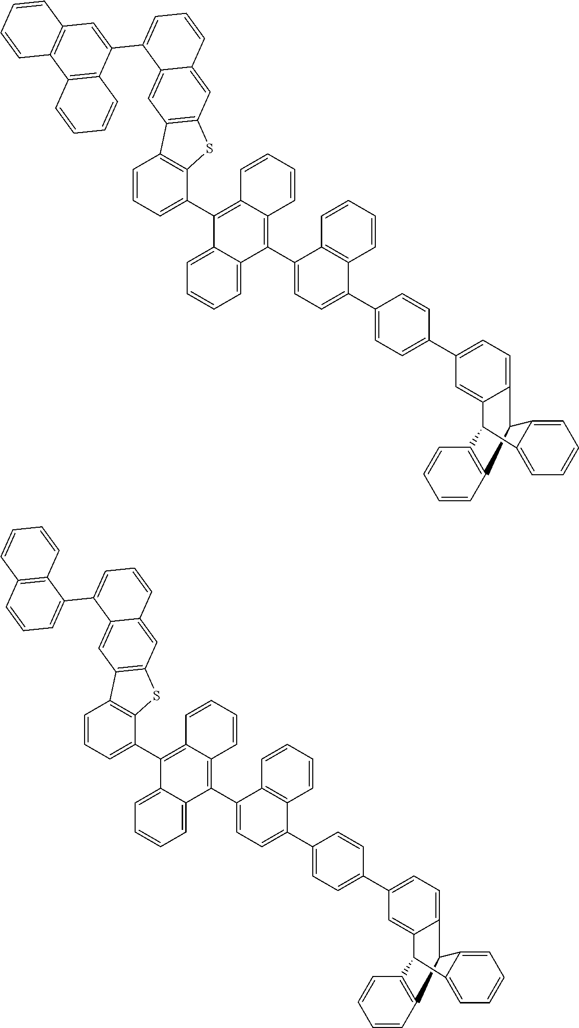 [SMILES: c1cc(-c2cc3ccccc3c3ccccc23)c2cc3c(cc2c1)sc1c(-c2c4ccccc4c(-c4ccc(-c5ccc(-c6ccc7c(c6)[C@H]6c8ccccc8[C@@H]7c7ccccc76)cc5)c5ccccc45)c4ccccc24)cccc13 ; c1ccc2c(-c3cccc4cc5sc6c(-c7c8ccccc8c(-c8ccc(-c9ccc(-c%10ccc%11c(c%10)[C@H]%10c%12ccccc%12[C@@H]%11c%11ccccc%11%10)cc9)c9ccccc89)c8ccccc78)cccc6c5cc34)cccc2c1]

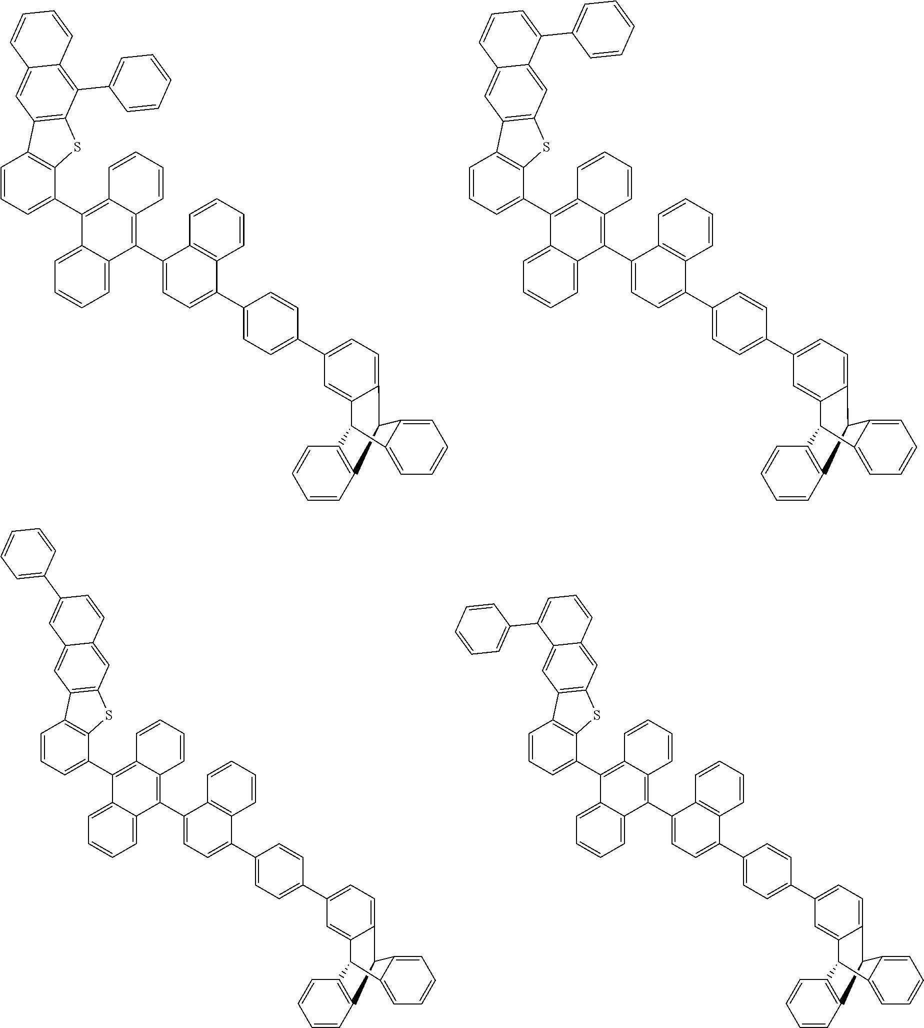

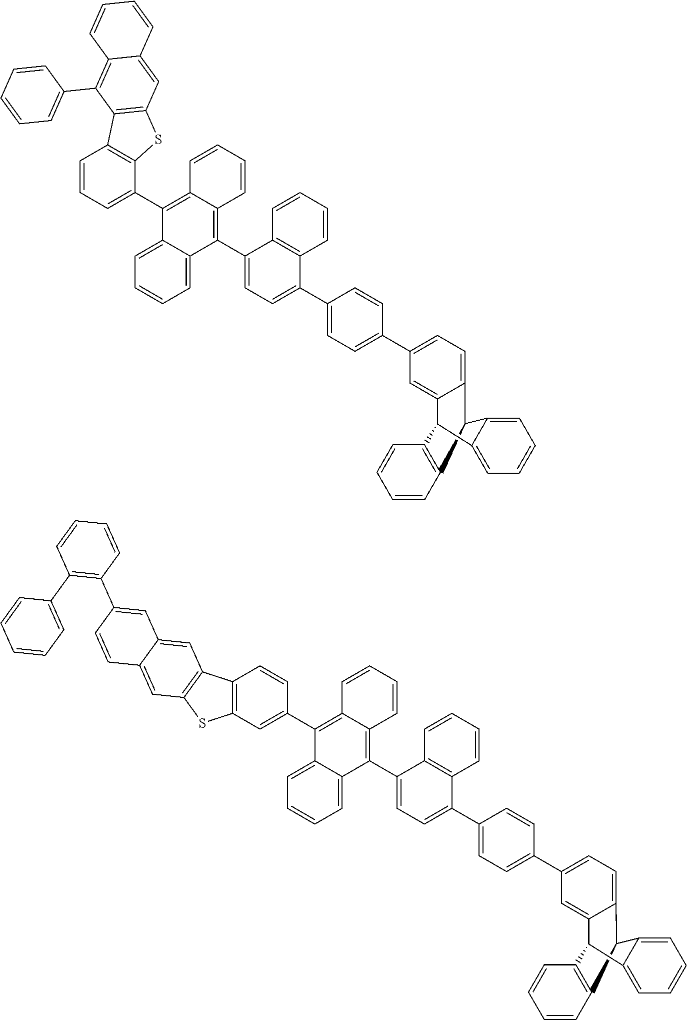 [SMILES: c1ccc(-c2c3ccccc3cc3sc4c(-c5c6ccccc6c(-c6ccc(-c7ccc(-c8ccc9c(c8)[C@H]8c%10ccccc%10[C@@H]9c9ccccc98)cc7)c7ccccc67)c6ccccc56)cccc4c23)cc1 ; c1ccc(-c2ccccc2-c2ccc3cc4sc5cc(-c6c7ccccc7c(-c7ccc(-c8ccc(-c9ccc%10c(c9)[C@H]9c%11ccccc%11[C@@H]%10c%10ccccc%109)cc8)c8ccccc78)c7ccccc67)ccc5c4cc3c2)cc1]

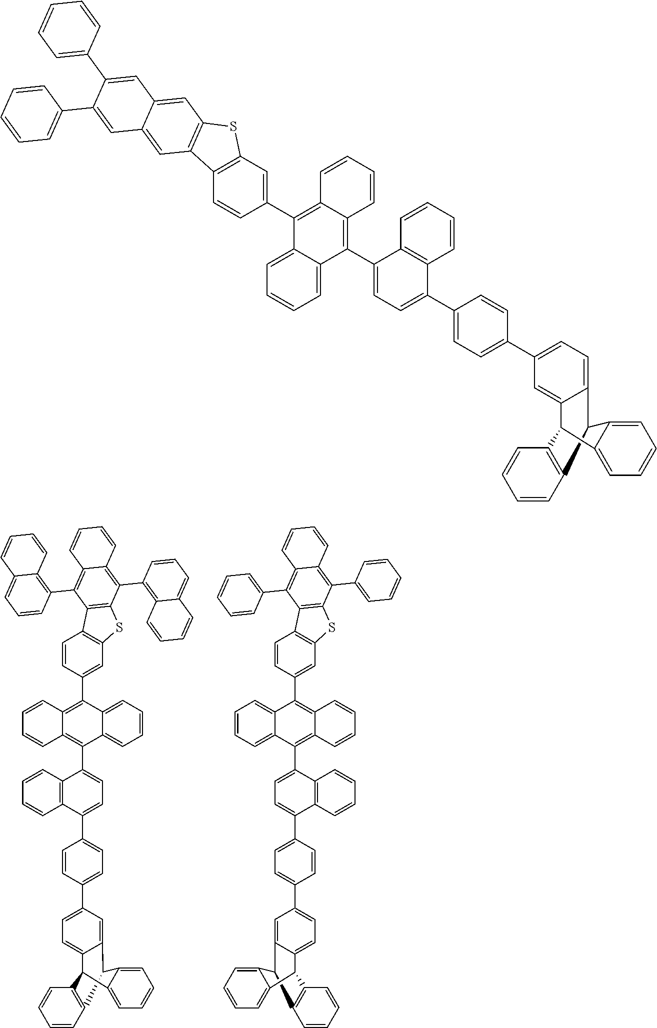

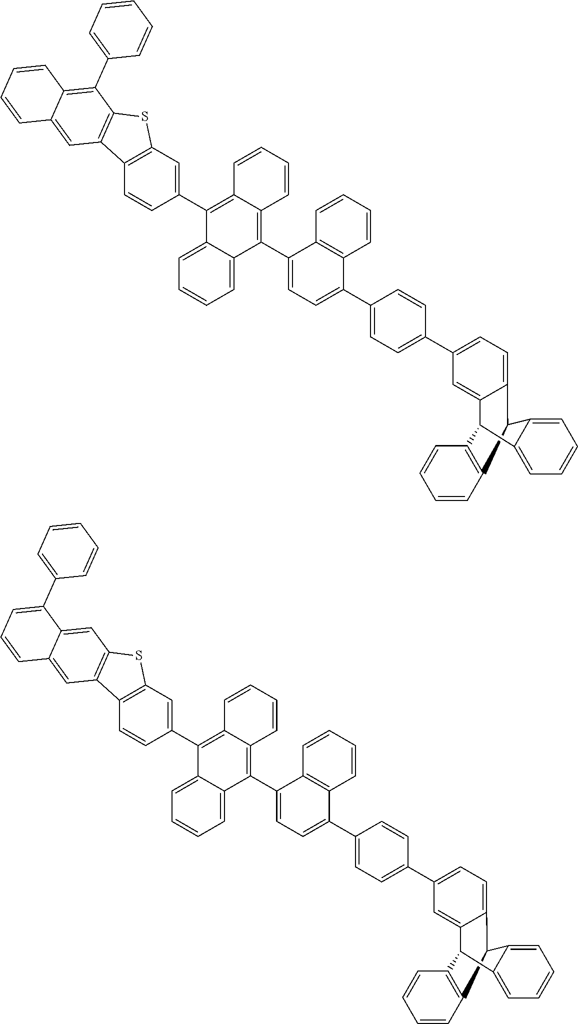 [SMILES: c1ccc(-c2c3ccccc3cc3c2sc2cc(-c4c5ccccc5c(-c5ccc(-c6ccc(-c7ccc8c(c7)[C@H]7c9ccccc9[C@@H]8c8ccccc87)cc6)c6ccccc56)c5ccccc45)ccc23)cc1 ; c1ccc(-c2cccc3cc4c(cc23)sc2cc(-c3c5ccccc5c(-c5ccc(-c6ccc(-c7ccc8c(c7)[C@H]7c9ccccc9[C@@H]8c8ccccc87)cc6)c6ccccc56)c5ccccc35)ccc24)cc1]

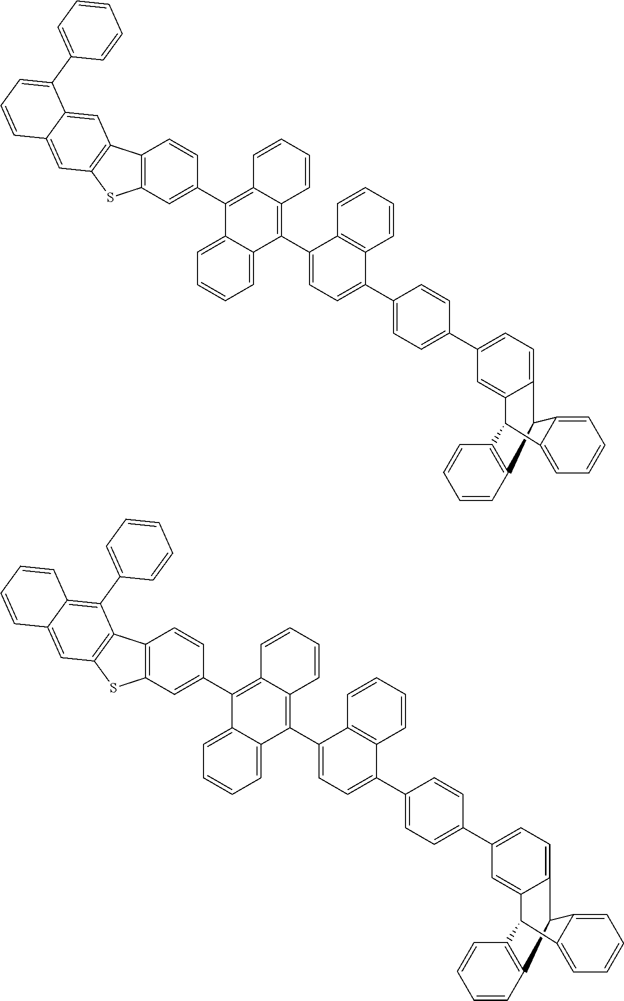 [SMILES: c1ccc(-c2cccc3cc4sc5cc(-c6c7ccccc7c(-c7ccc(-c8ccc(-c9ccc%10c(c9)[C@H]9c%11ccccc%11[C@@H]%10c%10ccccc%109)cc8)c8ccccc78)c7ccccc67)ccc5c4cc23)cc1 ; c1ccc(-c2c3ccccc3cc3sc4cc(-c5c6ccccc6c(-c6ccc(-c7ccc(-c8ccc9c(c8)[C@H]8c%10ccccc%10[C@@H]9c9ccccc98)cc7)c7ccccc67)c6ccccc56)ccc4c23)cc1]

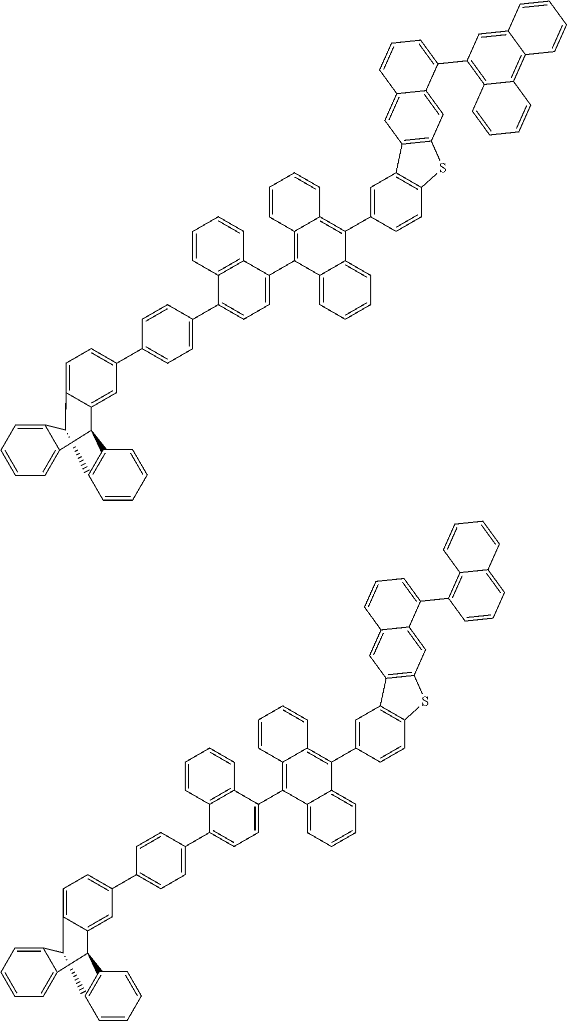 [SMILES: c1cc(-c2cc3ccccc3c3ccccc23)c2cc3sc4ccc(-c5c6ccccc6c(-c6ccc(-c7ccc(-c8ccc9c(c8)[C@H]8c%10ccccc%10[C@@H]9c9ccccc98)cc7)c7ccccc67)c6ccccc56)cc4c3cc2c1 ; c1ccc2c(-c3cccc4cc5c(cc34)sc3ccc(-c4c6ccccc6c(-c6ccc(-c7ccc(-c8ccc9c(c8)[C@H]8c%10ccccc%10[C@@H]9c9ccccc98)cc7)c7ccccc67)c6ccccc46)cc35)cccc2c1]

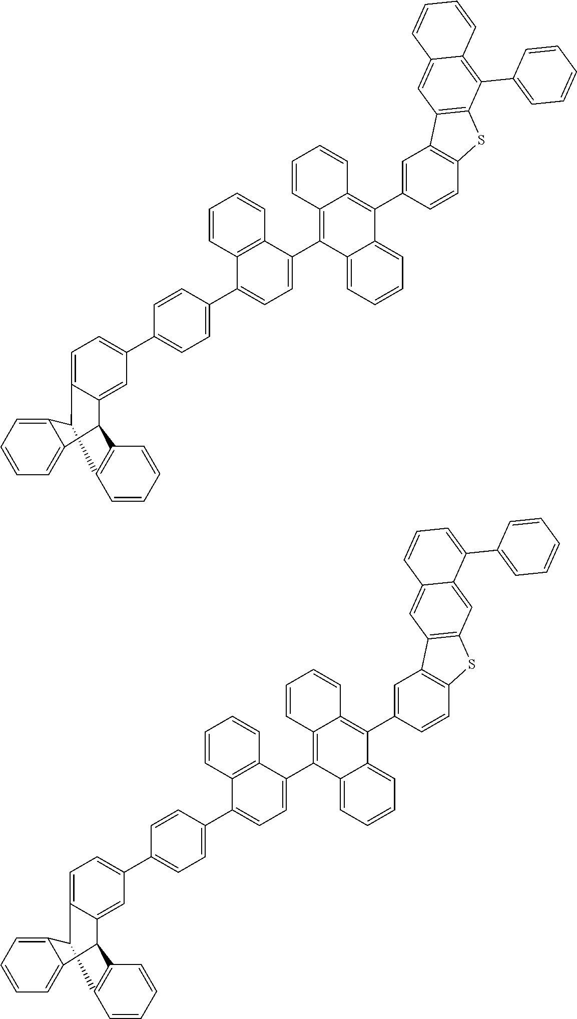 [SMILES: c1ccc(-c2c3ccccc3cc3c2sc2ccc(-c4c5ccccc5c(-c5ccc(-c6ccc(-c7ccc8c(c7)[C@H]7c9ccccc9[C@@H]8c8ccccc87)cc6)c6ccccc56)c5ccccc45)cc23)cc1 ; c1ccc(-c2cccc3cc4c(cc23)sc2ccc(-c3c5ccccc5c(-c5ccc(-c6ccc(-c7ccc8c(c7)[C@H]7c9ccccc9[C@@H]8c8ccccc87)cc6)c6ccccc56)c5ccccc35)cc24)cc1]

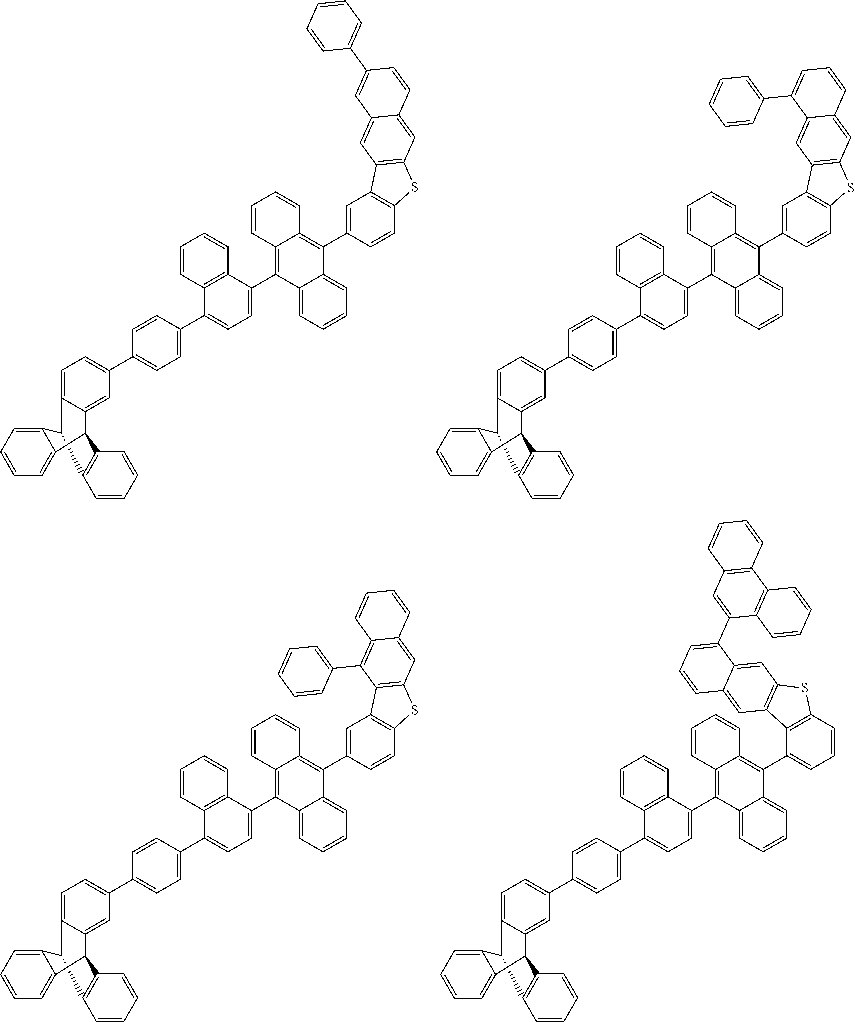

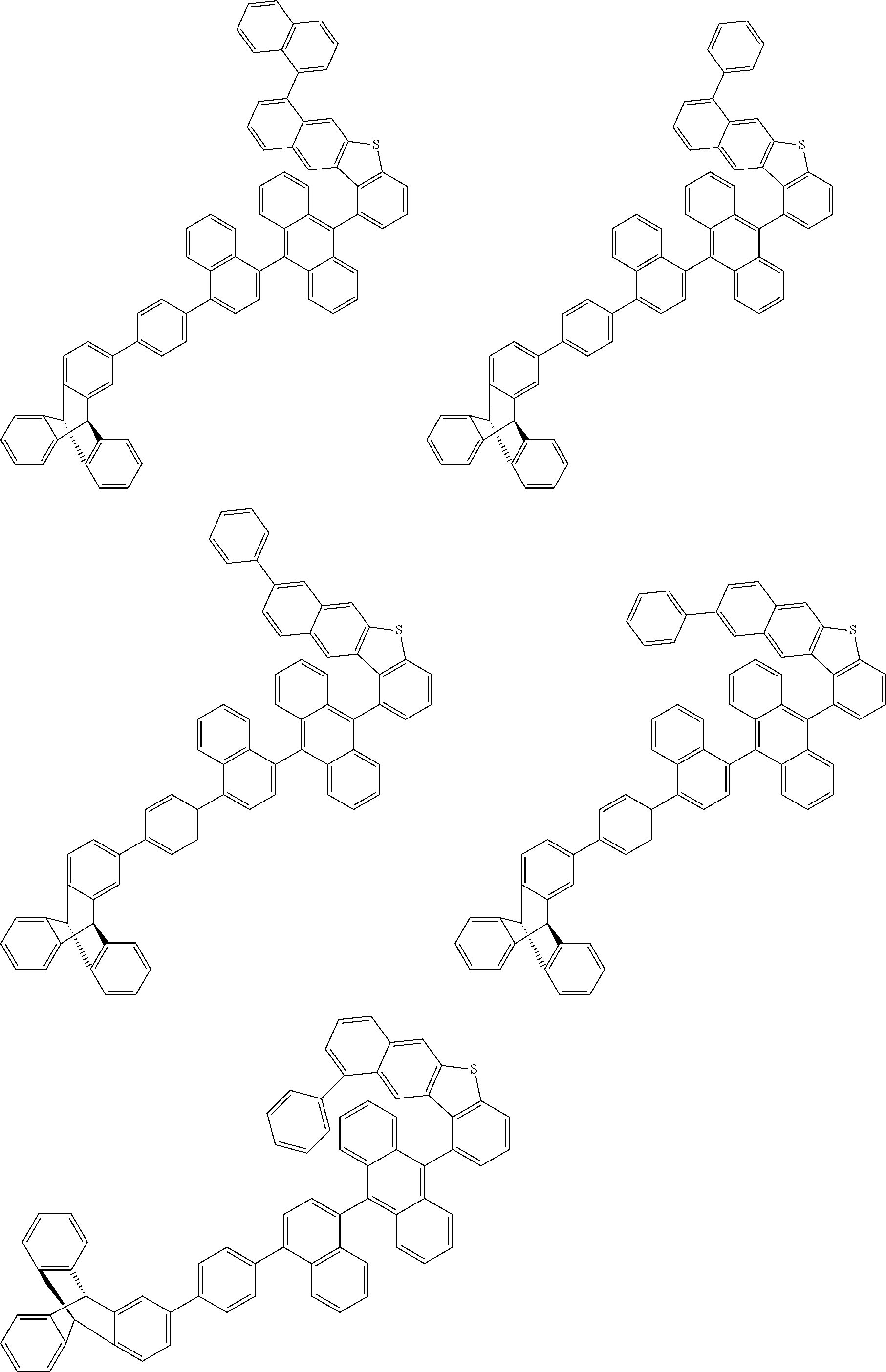

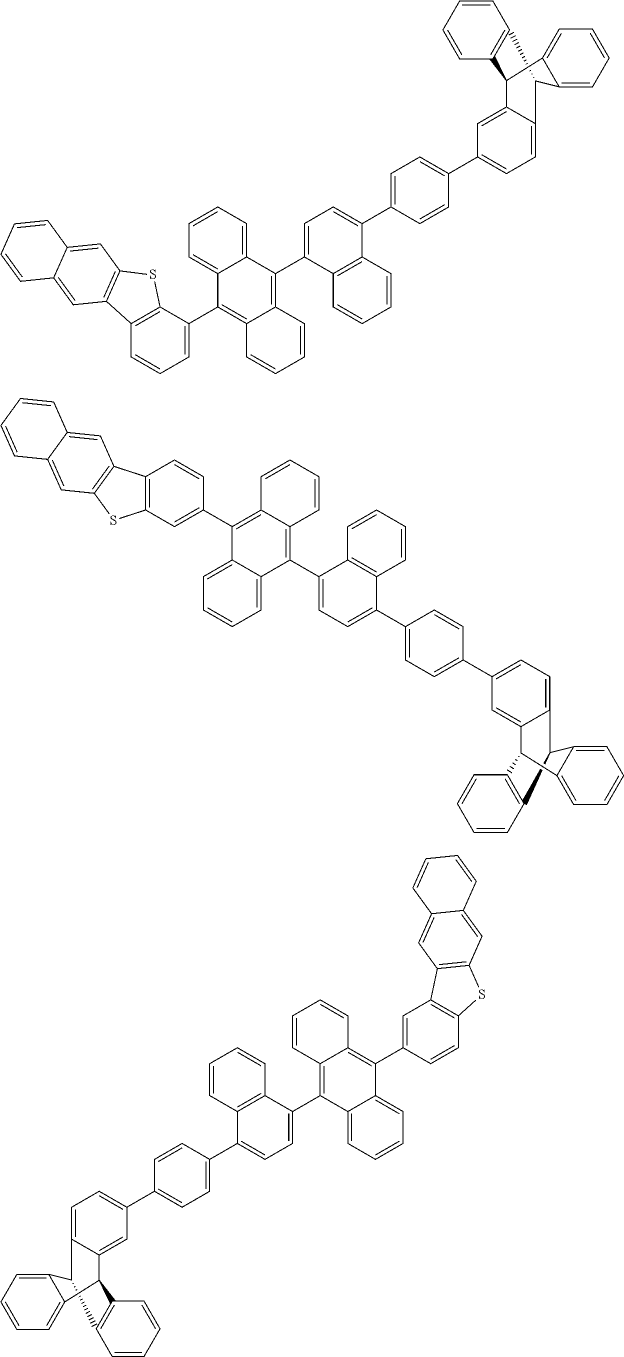

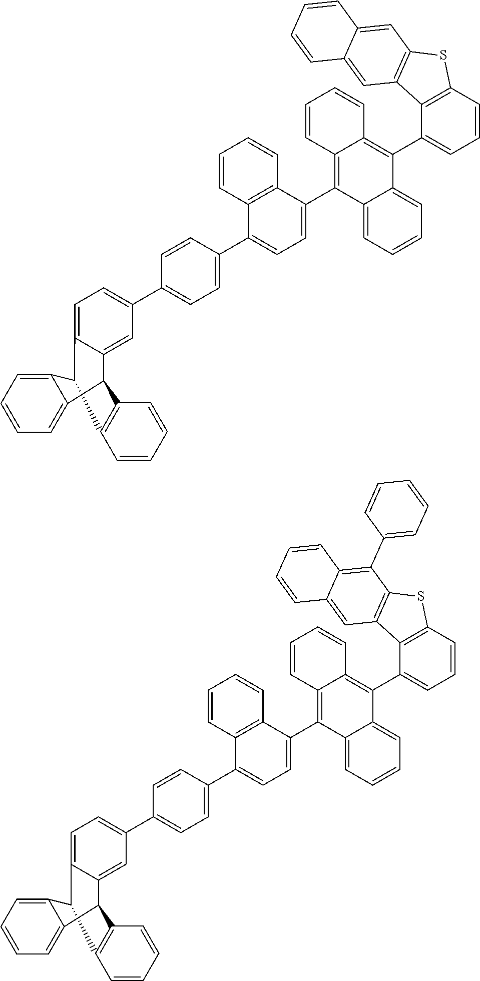 [SMILES: c1ccc2cc3c(cc2c1)sc1cccc(-c2c4ccccc4c(-c4ccc(-c5ccc(-c6ccc7c(c6)[C@H]6c8ccccc8[C@@H]7c7ccccc76)cc5)c5ccccc45)c4ccccc24)c13 ; c1ccc(-c2c3ccccc3cc3c2sc2cccc(-c4c5ccccc5c(-c5ccc(-c6ccc(-c7ccc8c(c7)[C@H]7c9ccccc9[C@@H]8c8ccccc87)cc6)c6ccccc56)c5ccccc45)c23)cc1]

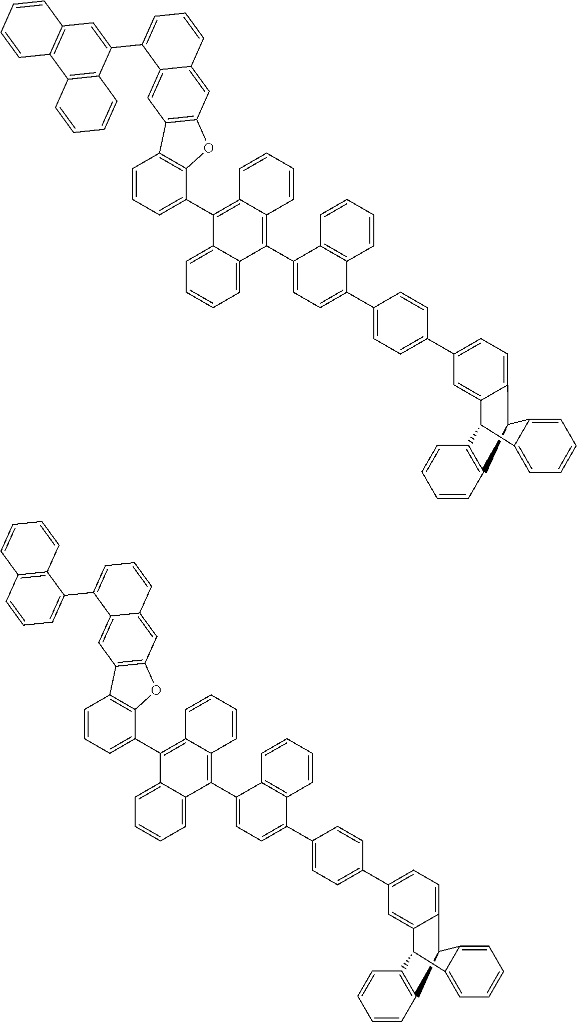 [SMILES: c1cc(-c2cc3ccccc3c3ccccc23)c2cc3c(cc2c1)oc1c(-c2c4ccccc4c(-c4ccc(-c5ccc(-c6ccc7c(c6)[C@H]6c8ccccc8[C@@H]7c7ccccc76)cc5)c5ccccc45)c4ccccc24)cccc13 ; c1ccc2c(-c3cccc4cc5oc6c(-c7c8ccccc8c(-c8ccc(-c9ccc(-c%10ccc%11c(c%10)[C@H]%10c%12ccccc%12[C@@H]%11c%11ccccc%11%10)cc9)c9ccccc89)c8ccccc78)cccc6c5cc34)cccc2c1]

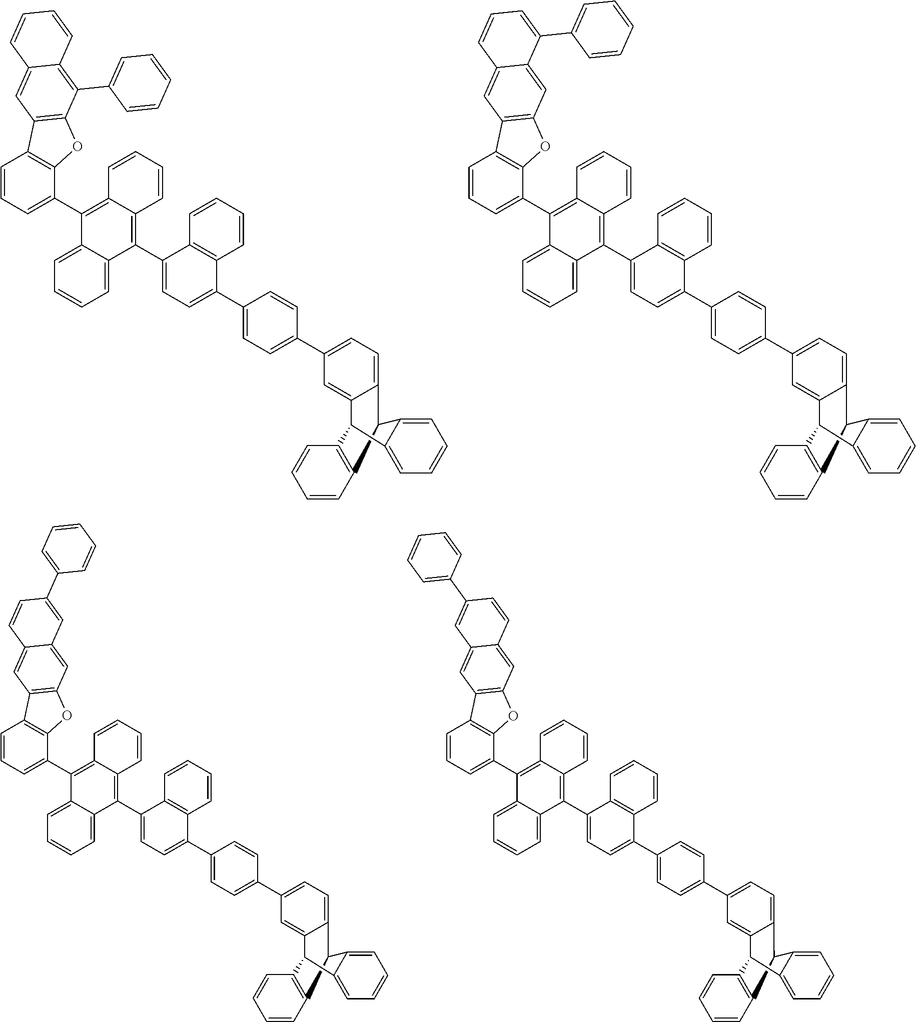

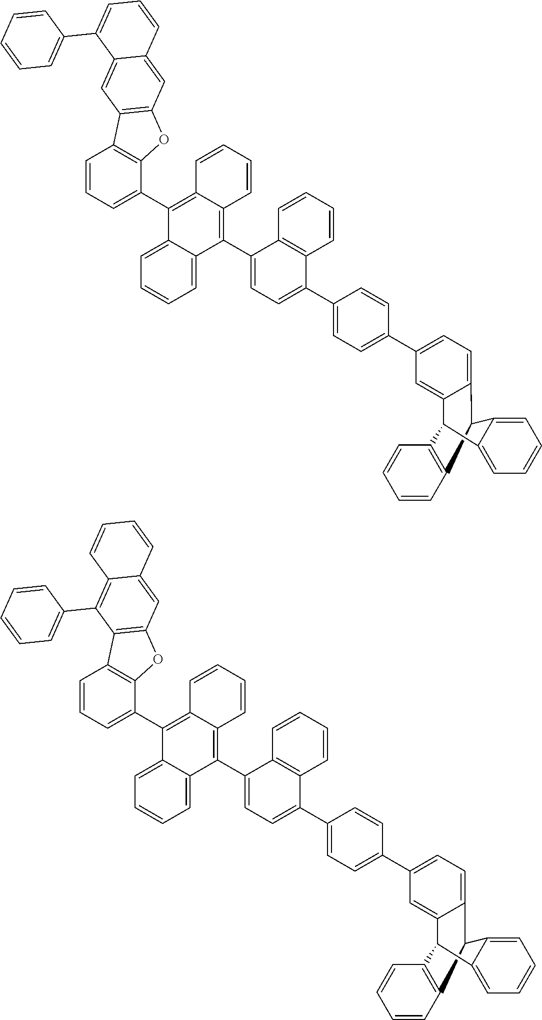 [SMILES: c1ccc(-c2cccc3cc4oc5c(-c6c7ccccc7c(-c7ccc(-c8ccc(-c9ccc%10c(c9)[C@H]9c%11ccccc%11[C@@H]%10c%10ccccc%109)cc8)c8ccccc78)c7ccccc67)cccc5c4cc23)cc1 ; c1ccc(-c2c3ccccc3cc3oc4c(-c5c6ccccc6c(-c6ccc(-c7ccc(-c8ccc9c(c8)[C@H]8c%10ccccc%10[C@@H]9c9ccccc98)cc7)c7ccccc67)c6ccccc56)cccc4c23)cc1]

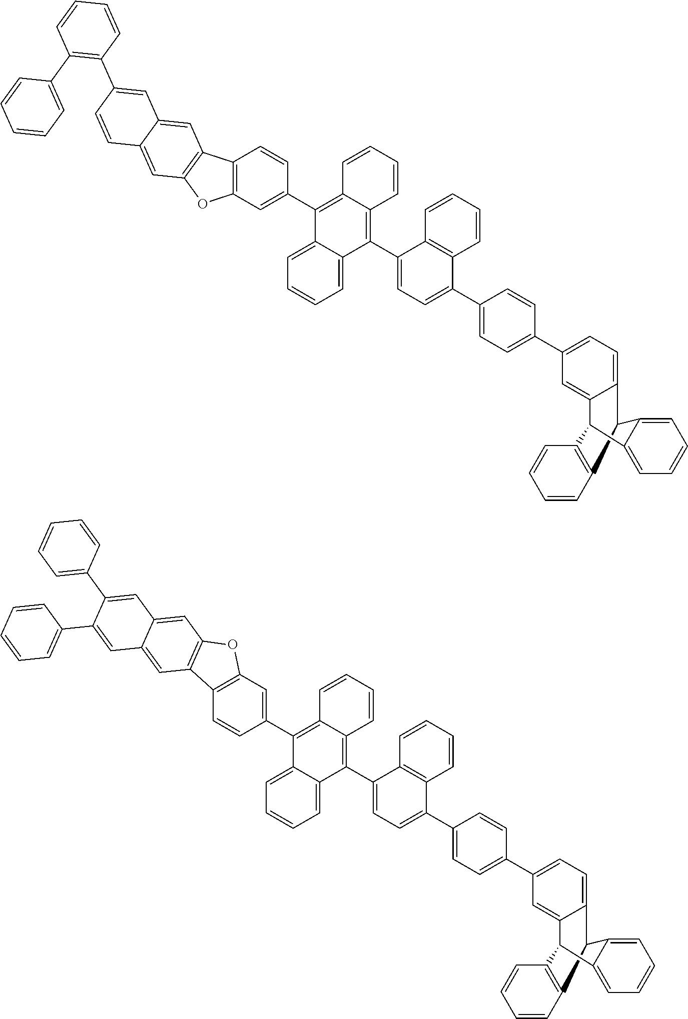 [SMILES: c1ccc(-c2ccccc2-c2ccc3cc4oc5cc(-c6c7ccccc7c(-c7ccc(-c8ccc(-c9ccc%10c(c9)[C@H]9c%11ccccc%11[C@@H]%10c%10ccccc%109)cc8)c8ccccc78)c7ccccc67)ccc5c4cc3c2)cc1 ; c1ccc(-c2cc3cc4oc5cc(-c6c7ccccc7c(-c7ccc(-c8ccc(-c9ccc%10c(c9)[C@H]9c%11ccccc%11[C@@H]%10c%10ccccc%109)cc8)c8ccccc78)c7ccccc67)ccc5c4cc3cc2-c2ccccc2)cc1]

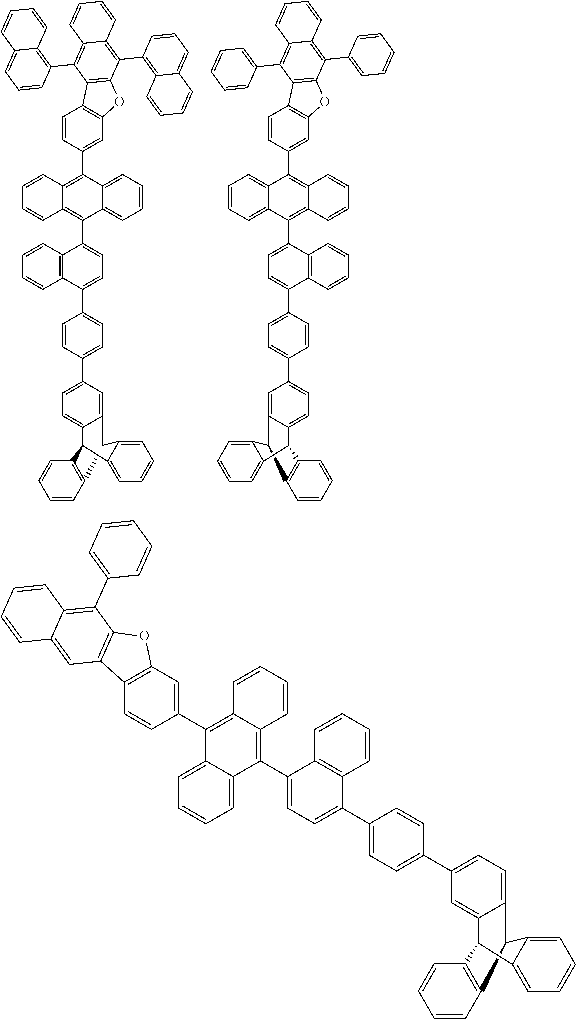

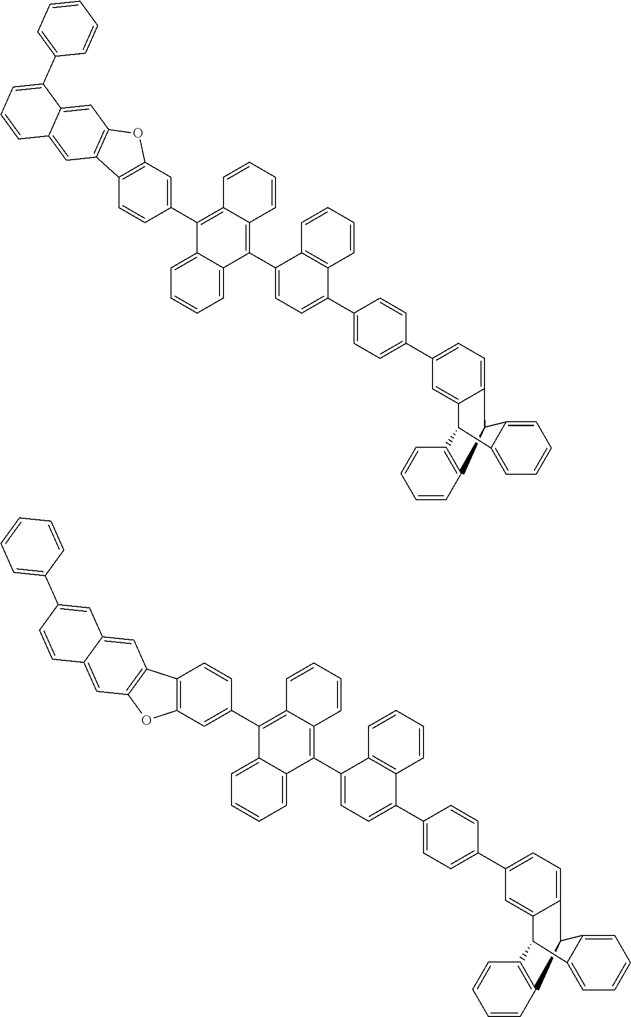 [SMILES: c1ccc(-c2cccc3cc4c(cc23)oc2cc(-c3c5ccccc5c(-c5ccc(-c6ccc(-c7ccc8c(c7)[C@H]7c9ccccc9[C@@H]8c8ccccc87)cc6)c6ccccc56)c5ccccc35)ccc24)cc1 ; c1ccc(-c2ccc3cc4oc5cc(-c6c7ccccc7c(-c7ccc(-c8ccc(-c9ccc%10c(c9)[C@H]9c%11ccccc%11[C@@H]%10c%10ccccc%109)cc8)c8ccccc78)c7ccccc67)ccc5c4cc3c2)cc1]

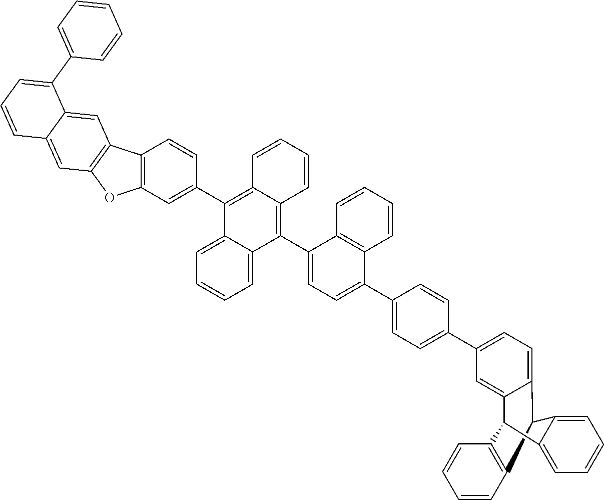 [SMILES: c1ccc(-c2cccc3cc4oc5cc(-c6c7ccccc7c(-c7ccc(-c8ccc(-c9ccc%10c(c9)[C@H]9c%11ccccc%11[C@@H]%10c%10ccccc%109)cc8)c8ccccc78)c7ccccc67)ccc5c4cc23)cc1]

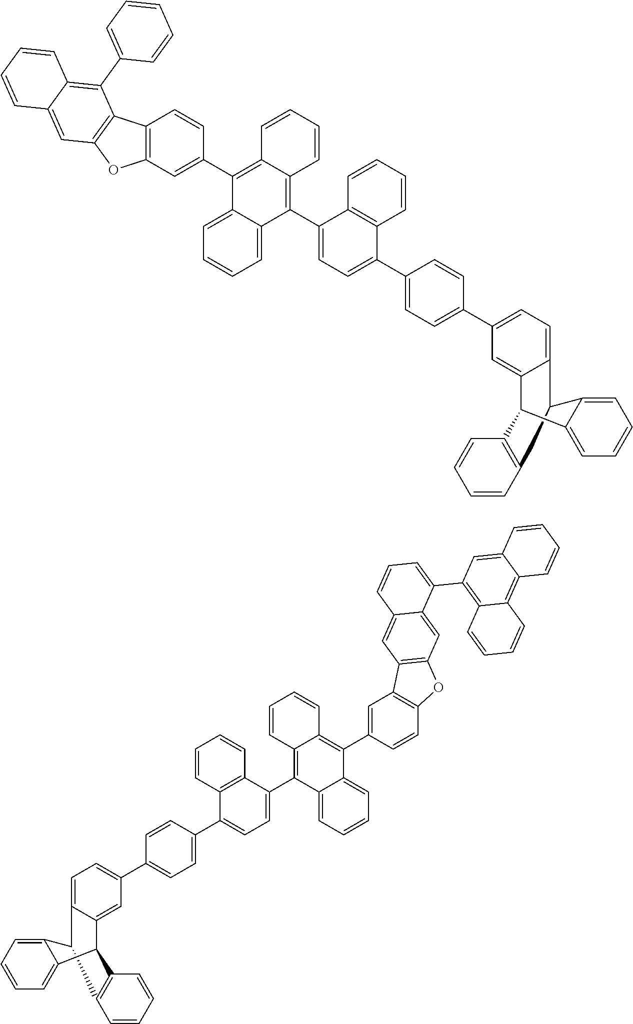

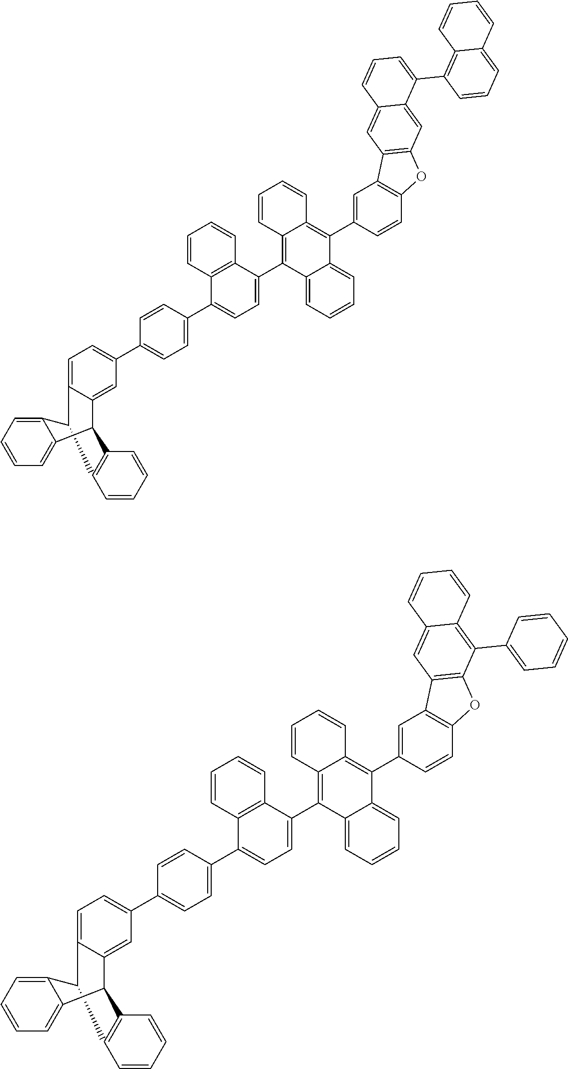 [SMILES: c1ccc2c(-c3cccc4cc5c(cc34)oc3ccc(-c4c6ccccc6c(-c6ccc(-c7ccc(-c8ccc9c(c8)[C@H]8c%10ccccc%10[C@@H]9c9ccccc98)cc7)c7ccccc67)c6ccccc46)cc35)cccc2c1 ; c1ccc(-c2c3ccccc3cc3c2oc2ccc(-c4c5ccccc5c(-c5ccc(-c6ccc(-c7ccc8c(c7)[C@H]7c9ccccc9[C@@H]8c8ccccc87)cc6)c6ccccc56)c5ccccc45)cc23)cc1]

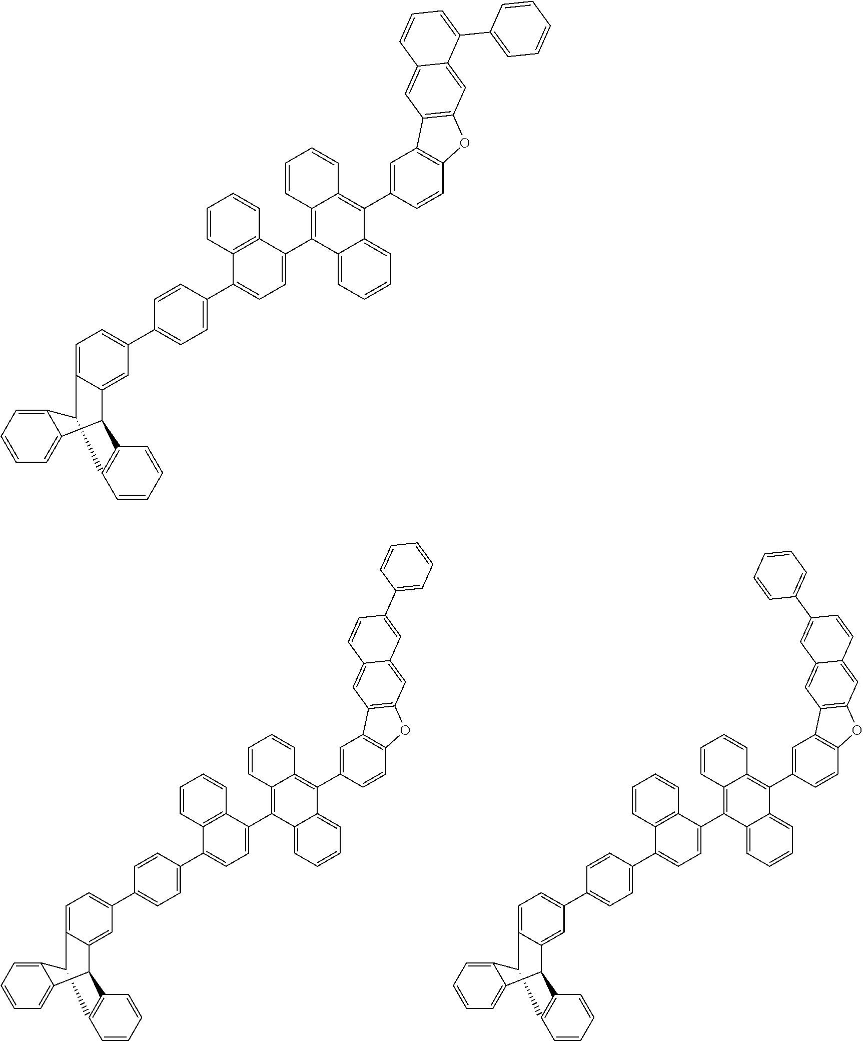 [SMILES: c1ccc(-c2cccc3cc4c(cc23)oc2ccc(-c3c5ccccc5c(-c5ccc(-c6ccc(-c7ccc8c(c7)[C@H]7c9ccccc9[C@@H]8c8ccccc87)cc6)c6ccccc56)c5ccccc35)cc24)cc1 ; c1ccc(-c2ccc3cc4c(cc3c2)oc2ccc(-c3c5ccccc5c(-c5ccc(-c6ccc(-c7ccc8c(c7)[C@H]7c9ccccc9[C@@H]8c8ccccc87)cc6)c6ccccc56)c5ccccc35)cc24)cc1 ; c1ccc(-c2ccc3cc4oc5ccc(-c6c7ccccc7c(-c7ccc(-c8ccc(-c9ccc%10c(c9)[C@H]9c%11ccccc%11[C@@H]%10c%10ccccc%109)cc8)c8ccccc78)c7ccccc67)cc5c4cc3c2)cc1]

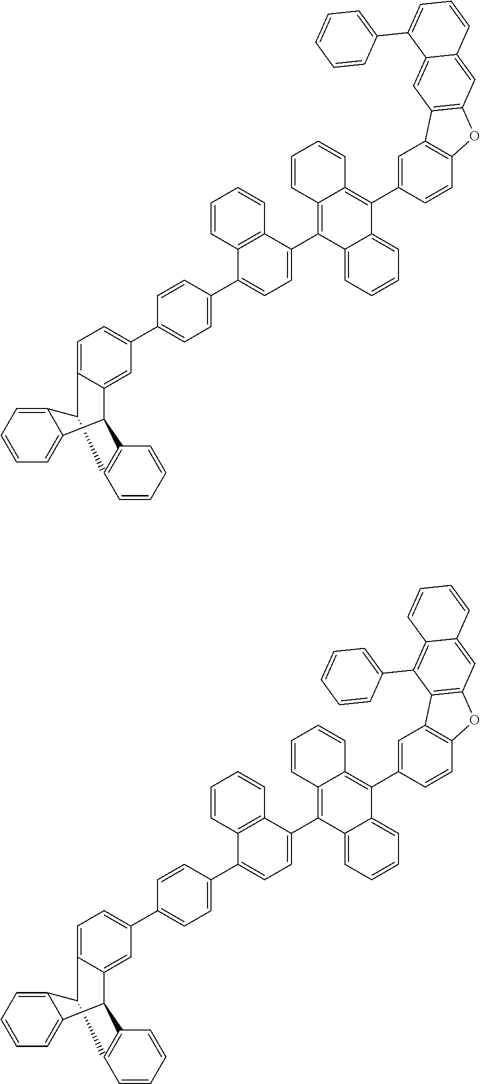 [SMILES: c1ccc(-c2cccc3cc4oc5ccc(-c6c7ccccc7c(-c7ccc(-c8ccc(-c9ccc%10c(c9)[C@H]9c%11ccccc%11[C@@H]%10c%10ccccc%109)cc8)c8ccccc78)c7ccccc67)cc5c4cc23)cc1 ; c1ccc(-c2c3ccccc3cc3oc4ccc(-c5c6ccccc6c(-c6ccc(-c7ccc(-c8ccc9c(c8)[C@H]8c%10ccccc%10[C@@H]9c9ccccc98)cc7)c7ccccc67)c6ccccc56)cc4c23)cc1]

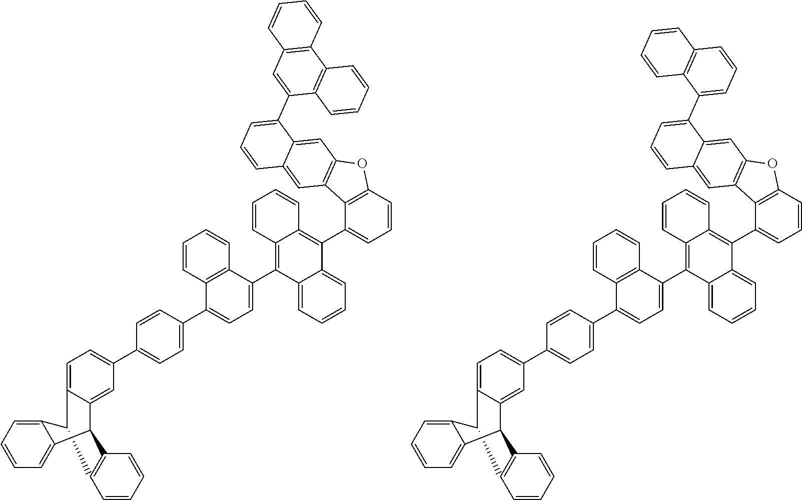 [SMILES: c1cc(-c2cc3ccccc3c3ccccc23)c2cc3oc4cccc(-c5c6ccccc6c(-c6ccc(-c7ccc(-c8ccc9c(c8)[C@H]8c%10ccccc%10[C@@H]9c9ccccc98)cc7)c7ccccc67)c6ccccc56)c4c3cc2c1 ; c1ccc2c(-c3cccc4cc5c(cc34)oc3cccc(-c4c6ccccc6c(-c6ccc(-c7ccc(-c8ccc9c(c8)[C@H]8c%10ccccc%10[C@@H]9c9ccccc98)cc7)c7ccccc67)c6ccccc46)c35)cccc2c1]

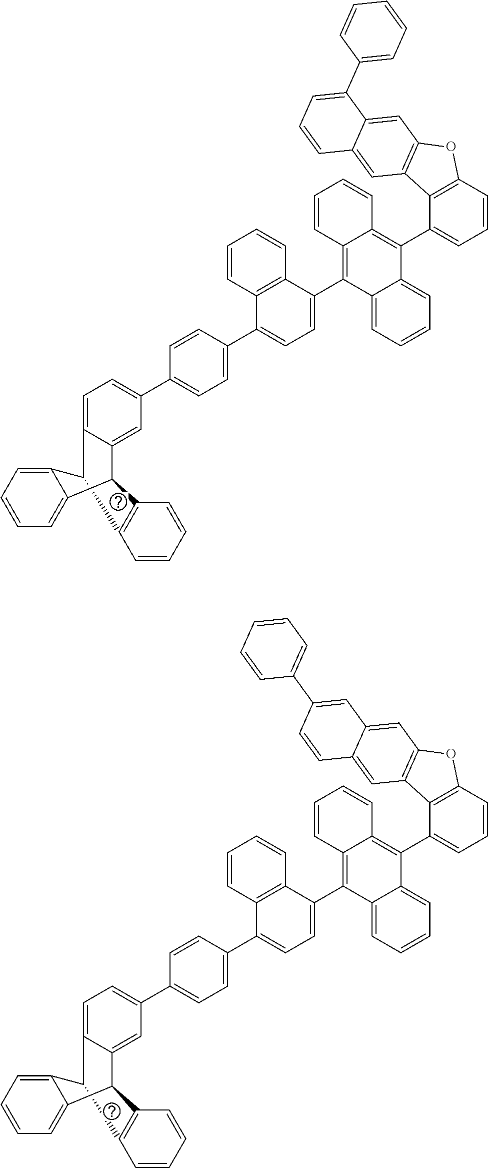 [SMILES: c1ccc(-c2cccc3cc4c(cc23)oc2cccc(-c3c5ccccc5c(-c5ccc(-c6ccc(-c7ccc8c(c7)[C@H]7c9ccccc9[C@@H]8c8ccccc87)cc6)c6ccccc56)c5ccccc35)c24)cc1 ; c1ccc(-c2ccc3cc4c(cc3c2)oc2cccc(-c3c5ccccc5c(-c5ccc(-c6ccc(-c7ccc8c(c7)[C@H]7c9ccccc9[C@@H]8c8ccccc87)cc6)c6ccccc56)c5ccccc35)c24)cc1]

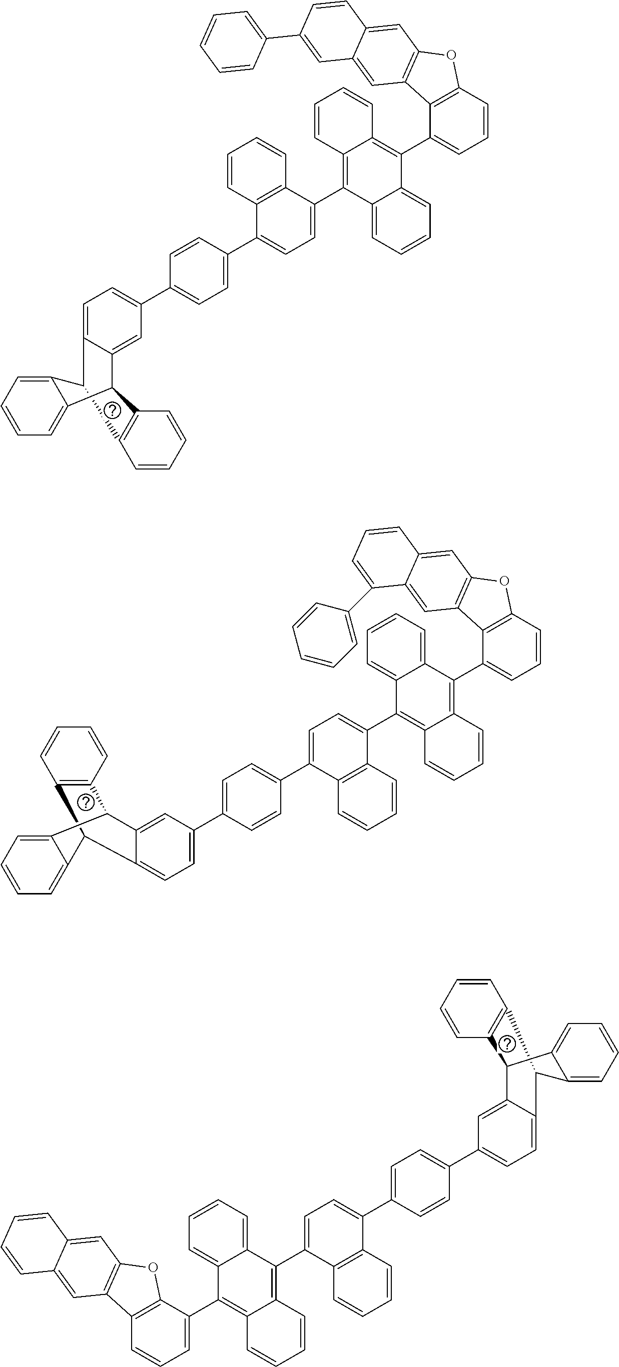 [SMILES: c1ccc(-c2ccc3cc4oc5cccc(-c6c7ccccc7c(-c7ccc(-c8ccc(-c9ccc%10c(c9)[C@H]9c%11ccccc%11[C@@H]%10c%10ccccc%109)cc8)c8ccccc78)c7ccccc67)c5c4cc3c2)cc1 ; c1ccc(-c2cccc3cc4oc5cccc(-c6c7ccccc7c(-c7ccc(-c8ccc(-c9ccc%10c(c9)[C@H]9c%11ccccc%11[C@@H]%10c%10ccccc%109)cc8)c8ccccc78)c7ccccc67)c5c4cc23)cc1 ; c1ccc2cc3c(cc2c1)oc1c(-c2c4ccccc4c(-c4ccc(-c5ccc(-c6ccc7c(c6)[C@H]6c8ccccc8[C@@H]7c7ccccc76)cc5)c5ccccc45)c4ccccc24)cccc13]

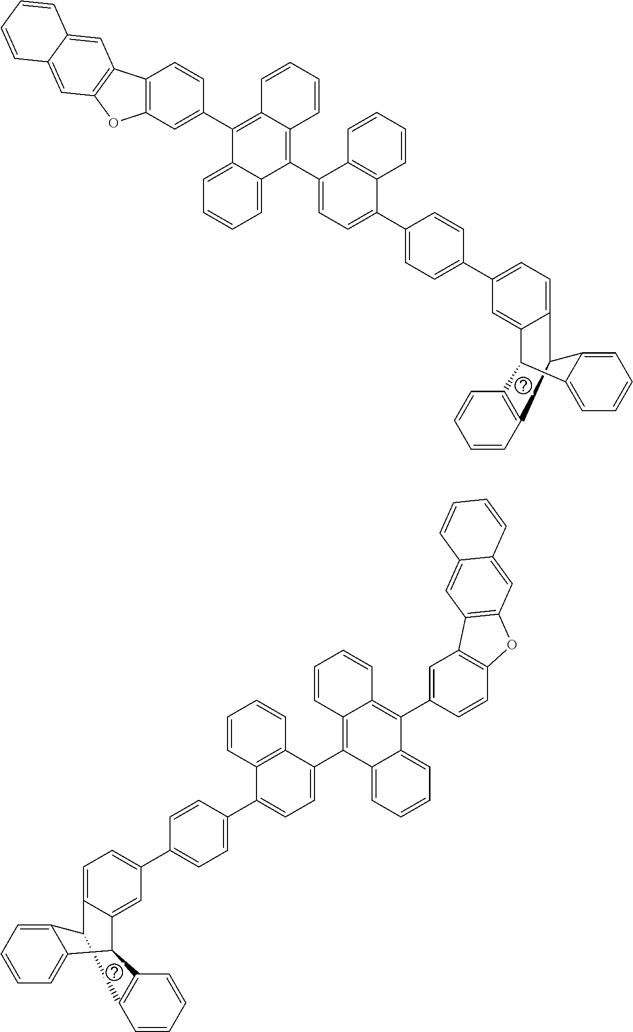 [SMILES: c1ccc2cc3c(cc2c1)oc1cc(-c2c4ccccc4c(-c4ccc(-c5ccc(-c6ccc7c(c6)[C@H]6c8ccccc8[C@@H]7c7ccccc76)cc5)c5ccccc45)c4ccccc24)ccc13 ; c1ccc2cc3c(cc2c1)oc1ccc(-c2c4ccccc4c(-c4ccc(-c5ccc(-c6ccc7c(c6)[C@H]6c8ccccc8[C@@H]7c7ccccc76)cc5)c5ccccc45)c4ccccc24)cc13]

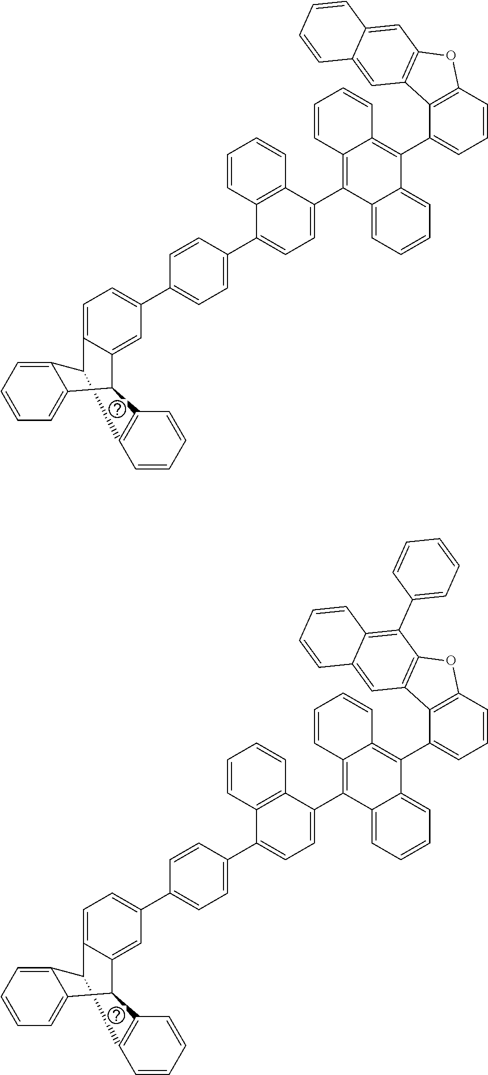 [SMILES: c1ccc2cc3c(cc2c1)oc1cccc(-c2c4ccccc4c(-c4ccc(-c5ccc(-c6ccc7c(c6)[C@H]6c8ccccc8[C@@H]7c7ccccc76)cc5)c5ccccc45)c4ccccc24)c13 ; c1ccc(-c2c3ccccc3cc3c2oc2cccc(-c4c5ccccc5c(-c5ccc(-c6ccc(-c7ccc8c(c7)[C@H]7c9ccccc9[C@@H]8c8ccccc87)cc6)c6ccccc56)c5ccccc45)c23)cc1]

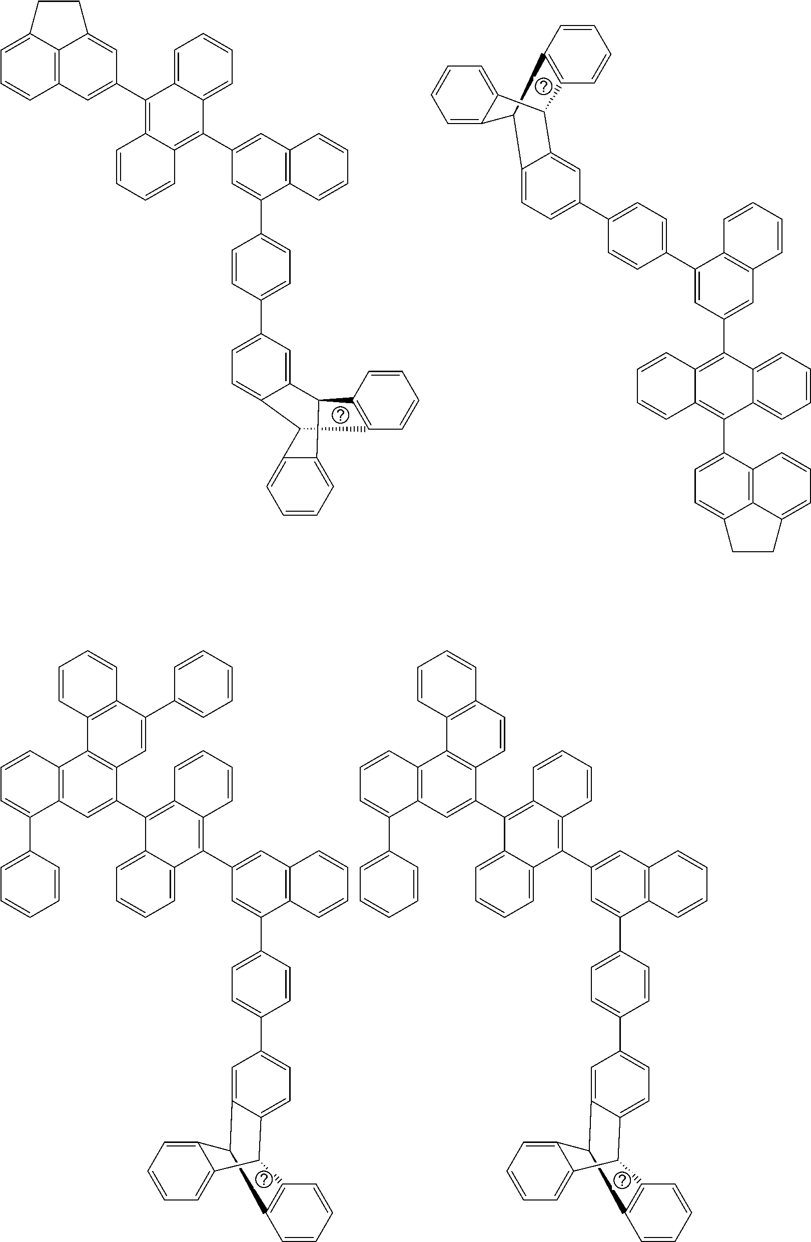

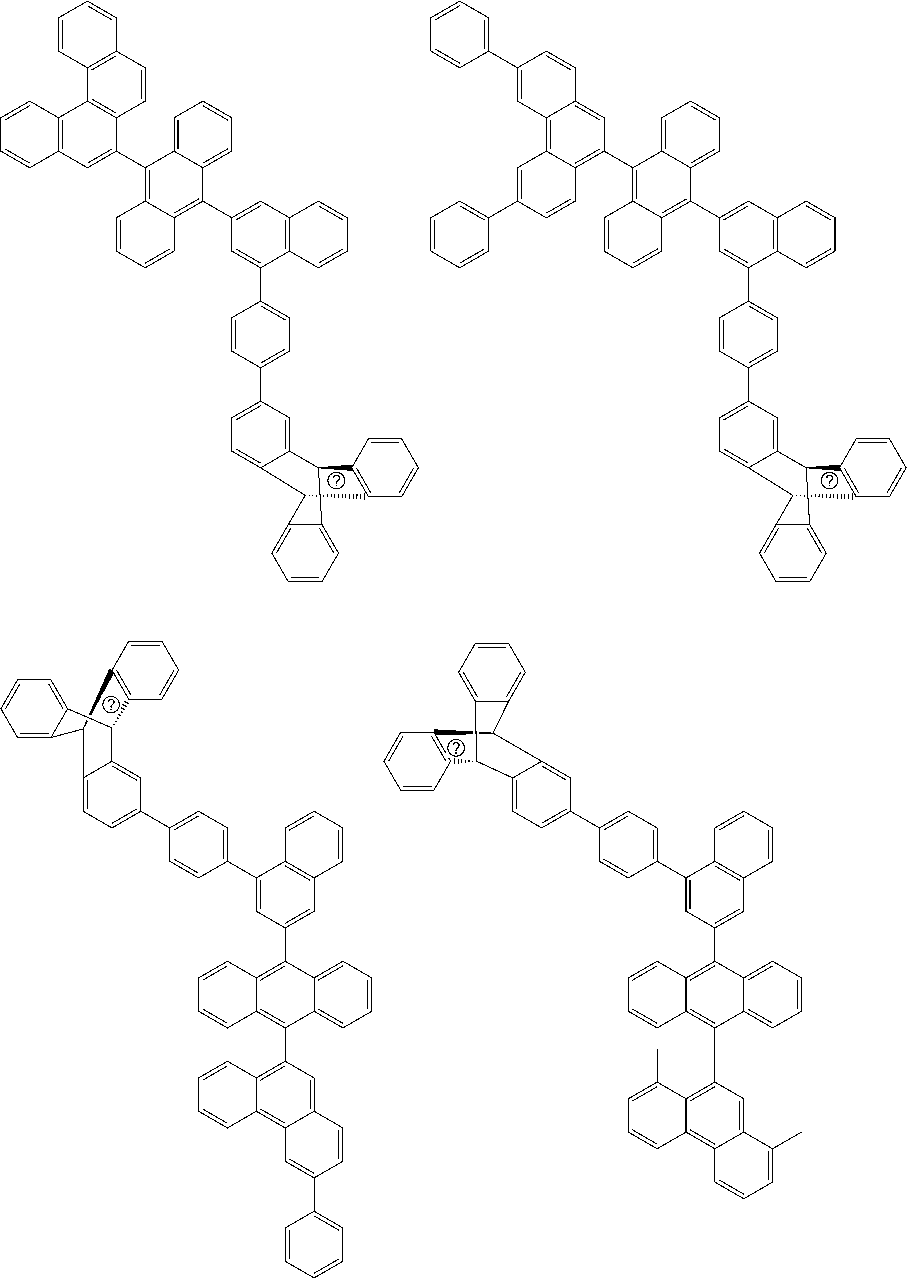

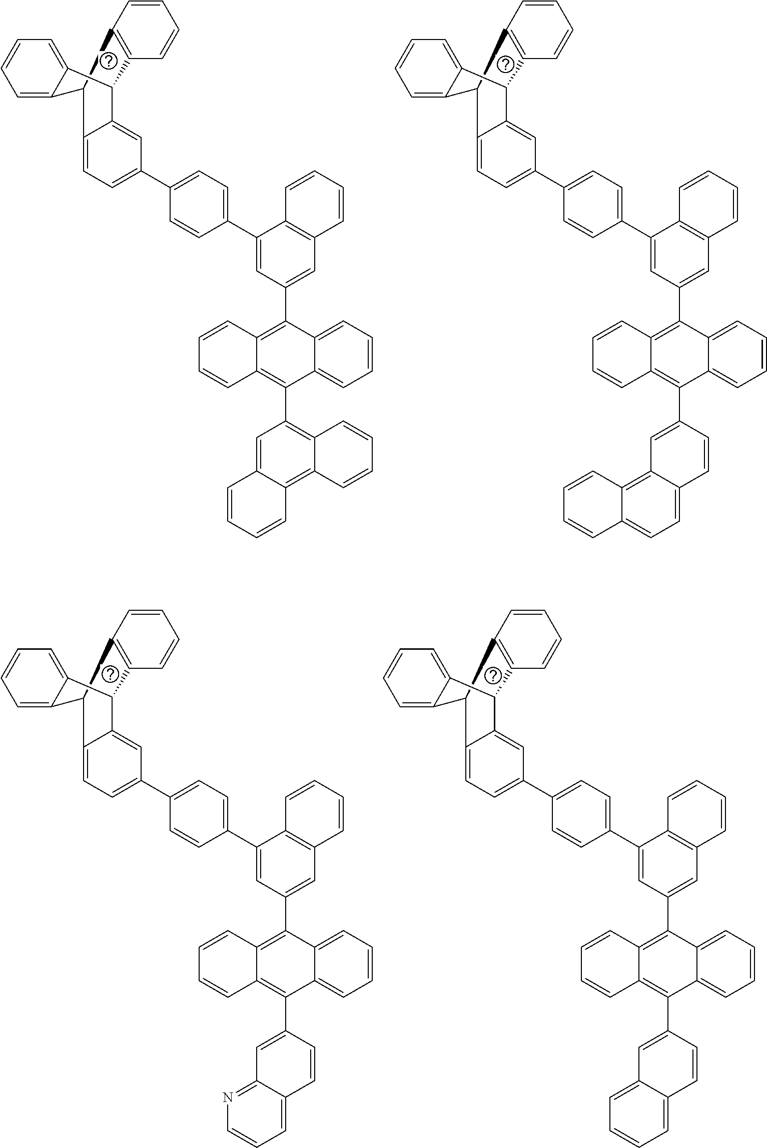

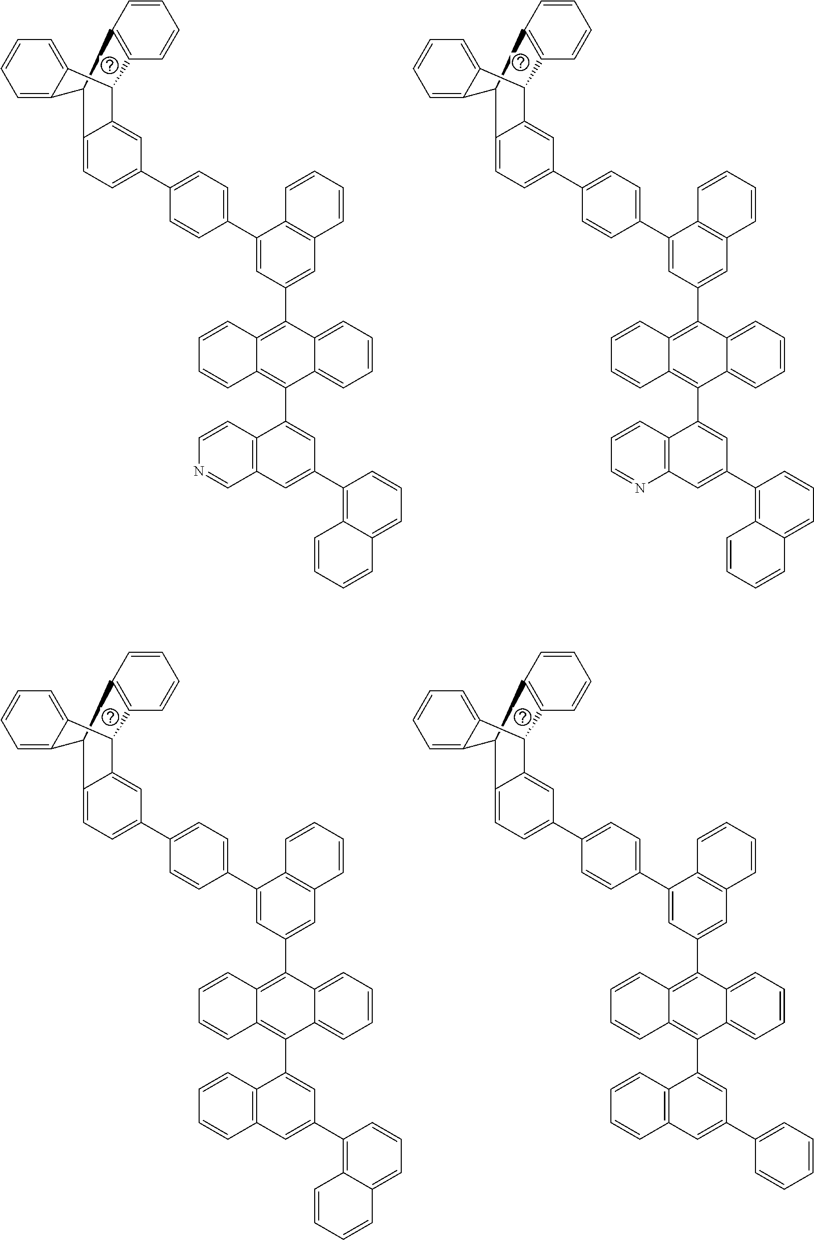

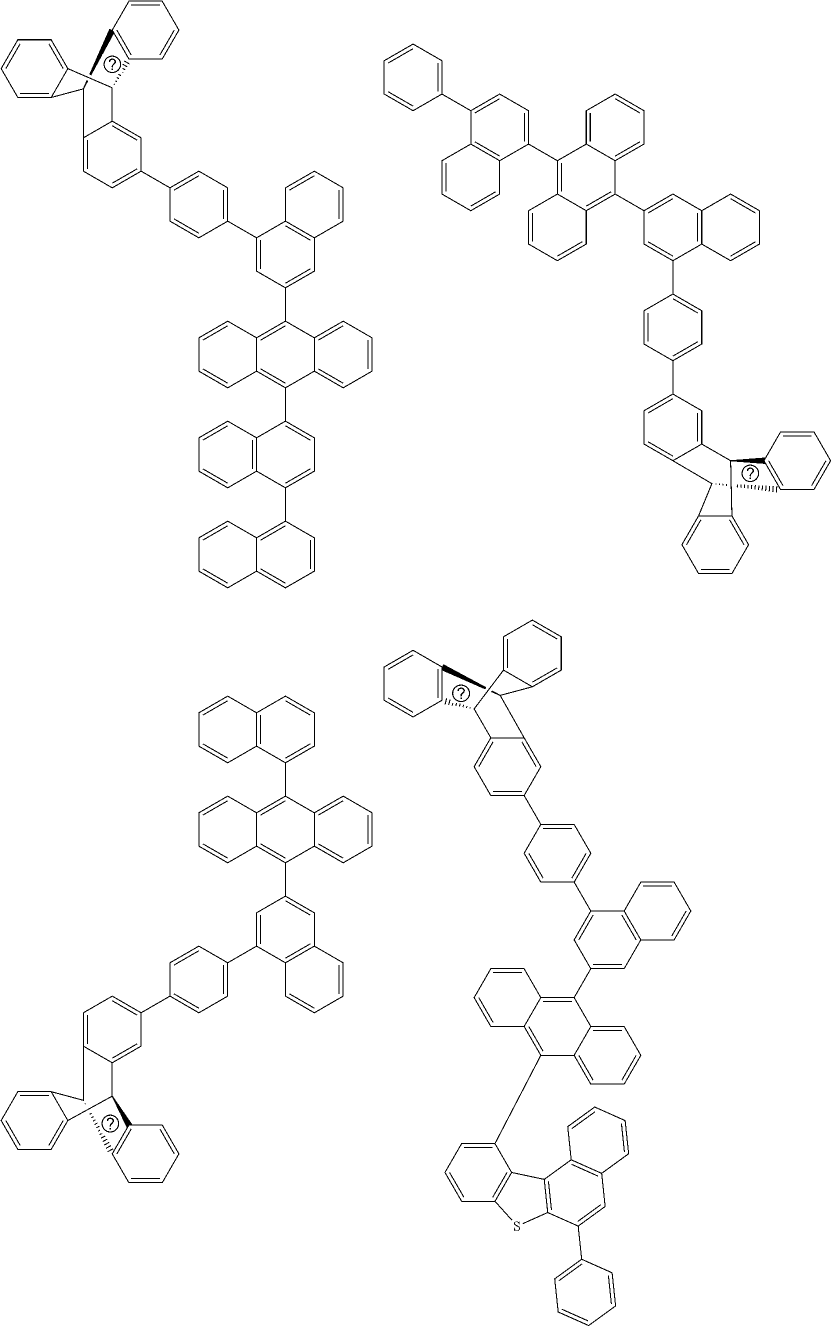

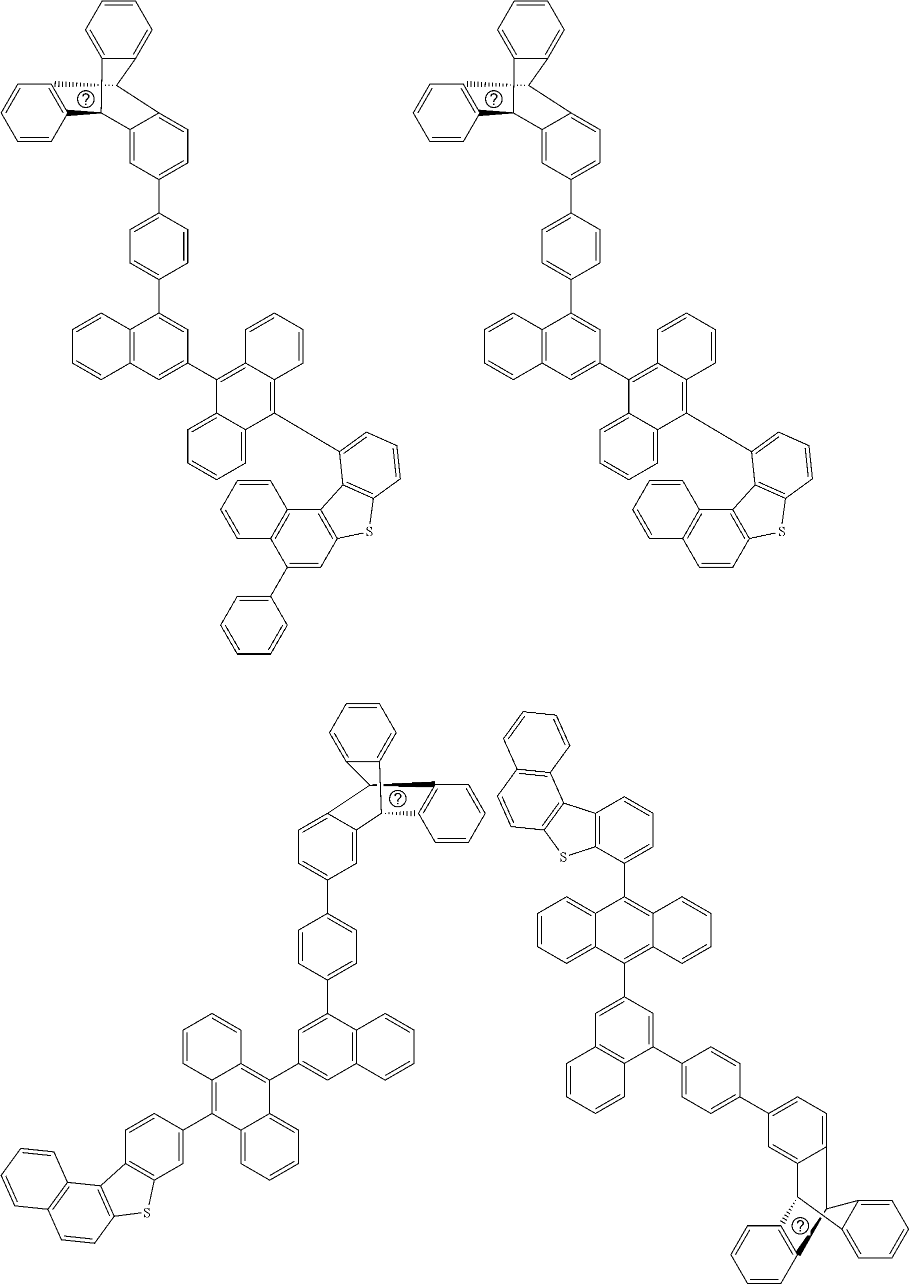

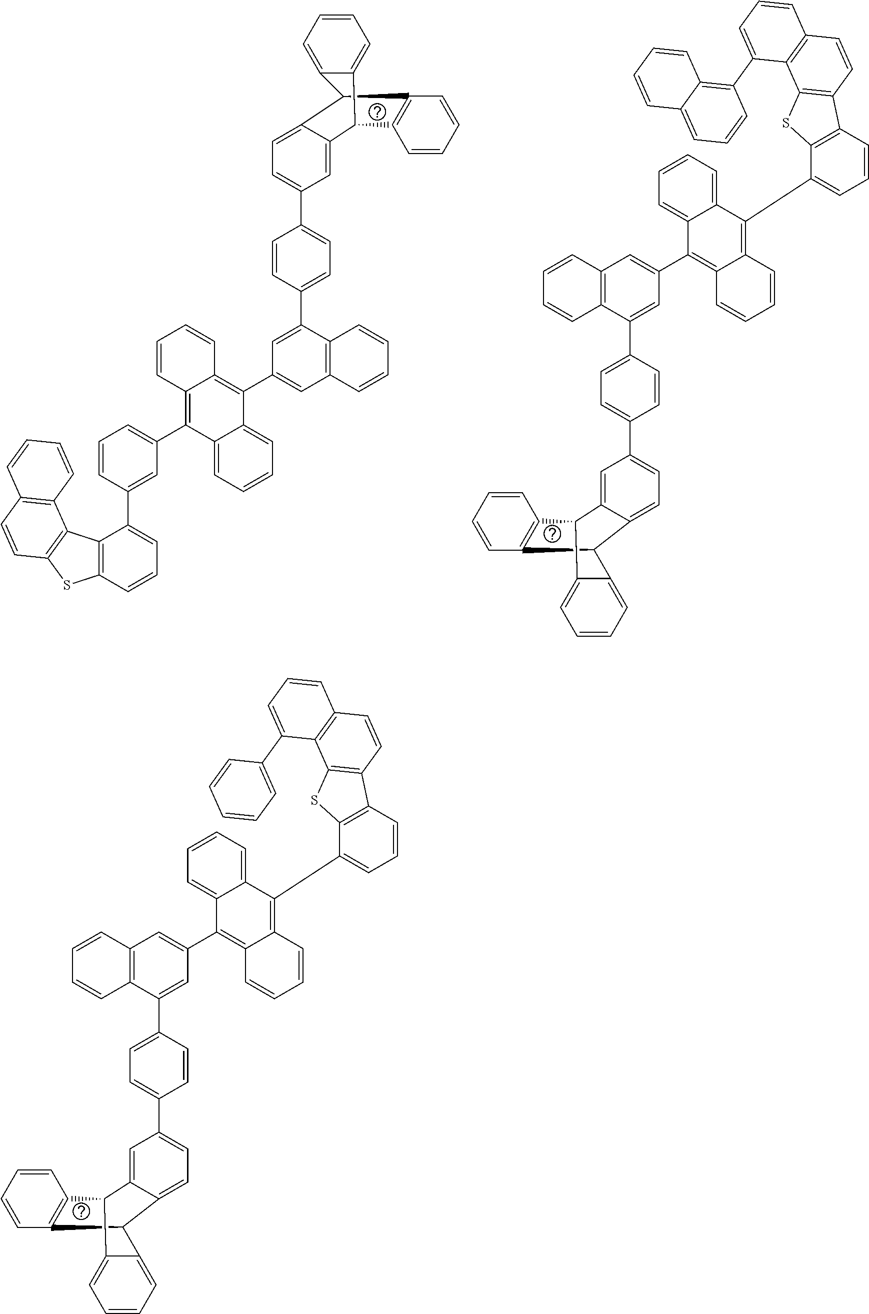 [SMILES: c1cc(-c2c3ccccc3c(-c3cc(-c4ccc(-c5ccc6c(c5)[C@H]5c7ccccc7[C@@H]6c6ccccc65)cc4)c4ccccc4c3)c3ccccc23)cc(-c2cccc3sc4ccc5ccccc5c4c23)c1 ; c1ccc2c(-c3ccc(-c4ccc5c(c4)[C@H]4c6ccccc6[C@@H]5c5ccccc54)cc3)cc(-c3c4ccccc4c(-c4cccc5c4sc4c5ccc5cccc(-c6cccc7ccccc67)c54)c4ccccc34)cc2c1 ; c1ccc(-c2cccc3ccc4c5cccc(-c6c7ccccc7c(-c7cc(-c8ccc(-c9ccc%10c(c9)[C@H]9c%11ccccc%11[C@@H]%10c%10ccccc%109)cc8)c8ccccc8c7)c7ccccc67)c5sc4c23)cc1]

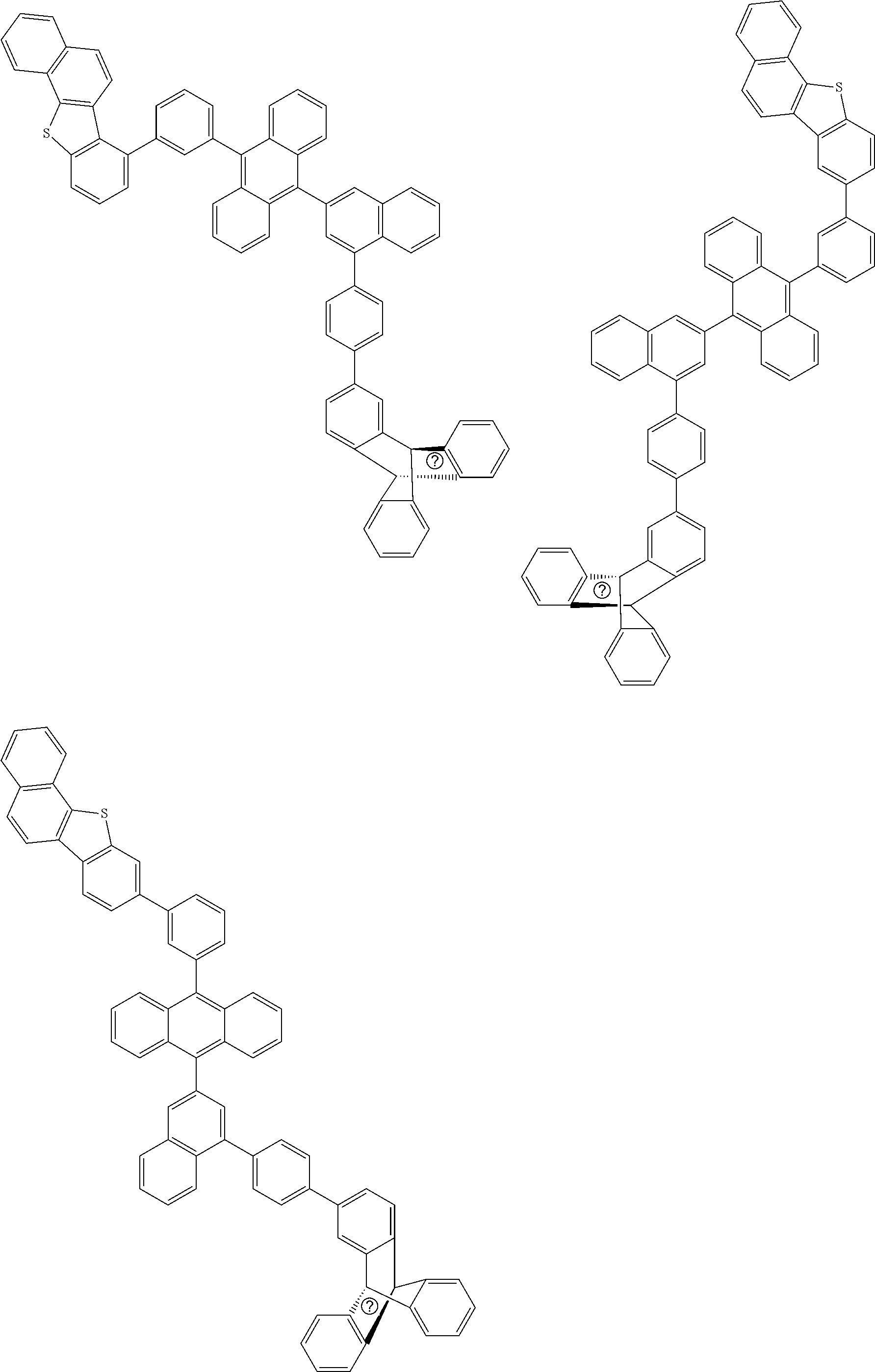 [SMILES: c1cc(-c2c3ccccc3c(-c3cc(-c4ccc(-c5ccc6c(c5)[C@H]5c7ccccc7[C@@H]6c6ccccc65)cc4)c4ccccc4c3)c3ccccc23)cc(-c2cccc3sc4c5ccccc5ccc4c23)c1 ; c1cc(-c2ccc3sc4c5ccccc5ccc4c3c2)cc(-c2c3ccccc3c(-c3cc(-c4ccc(-c5ccc6c(c5)[C@H]5c7ccccc7[C@@H]6c6ccccc65)cc4)c4ccccc4c3)c3ccccc23)c1 ; c1cc(-c2ccc3c(c2)sc2c4ccccc4ccc32)cc(-c2c3ccccc3c(-c3cc(-c4ccc(-c5ccc6c(c5)[C@H]5c7ccccc7[C@@H]6c6ccccc65)cc4)c4ccccc4c3)c3ccccc23)c1]

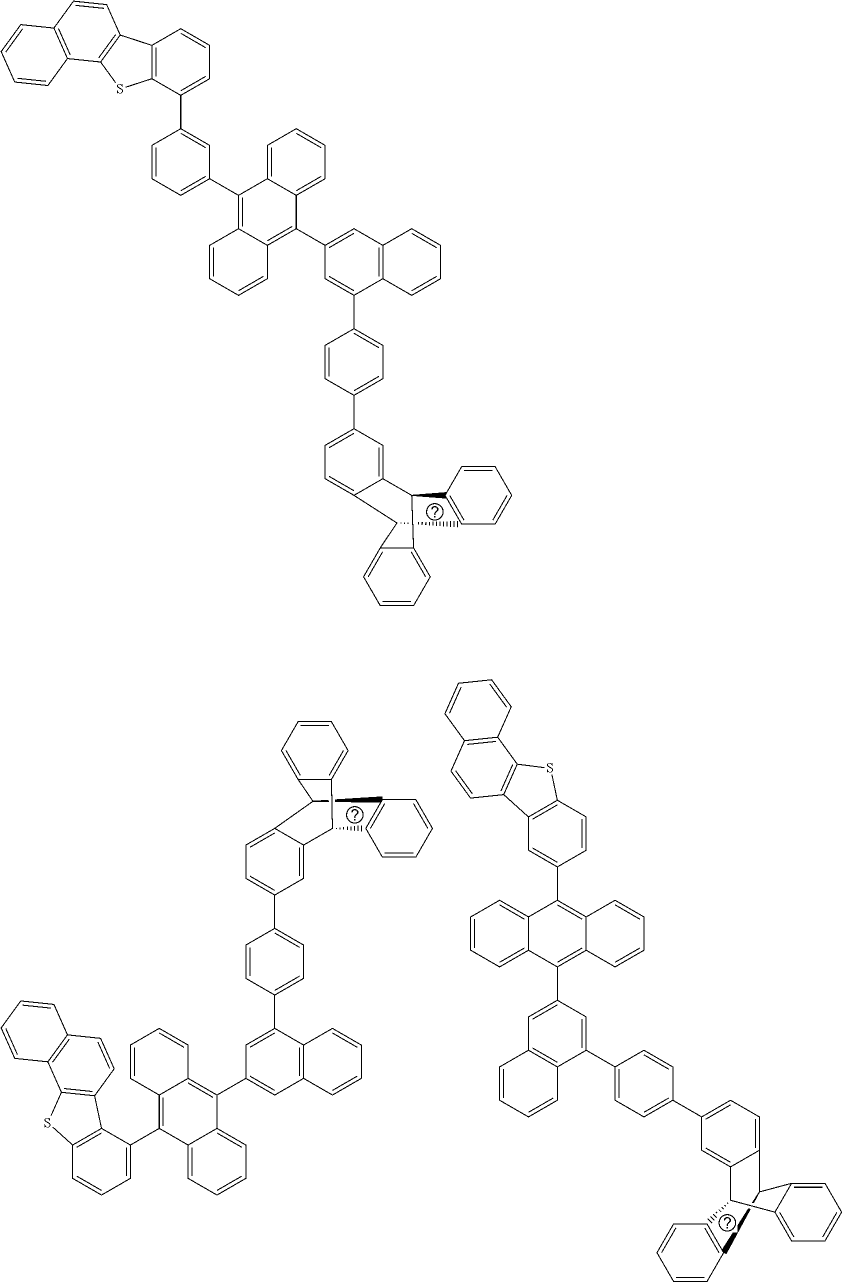

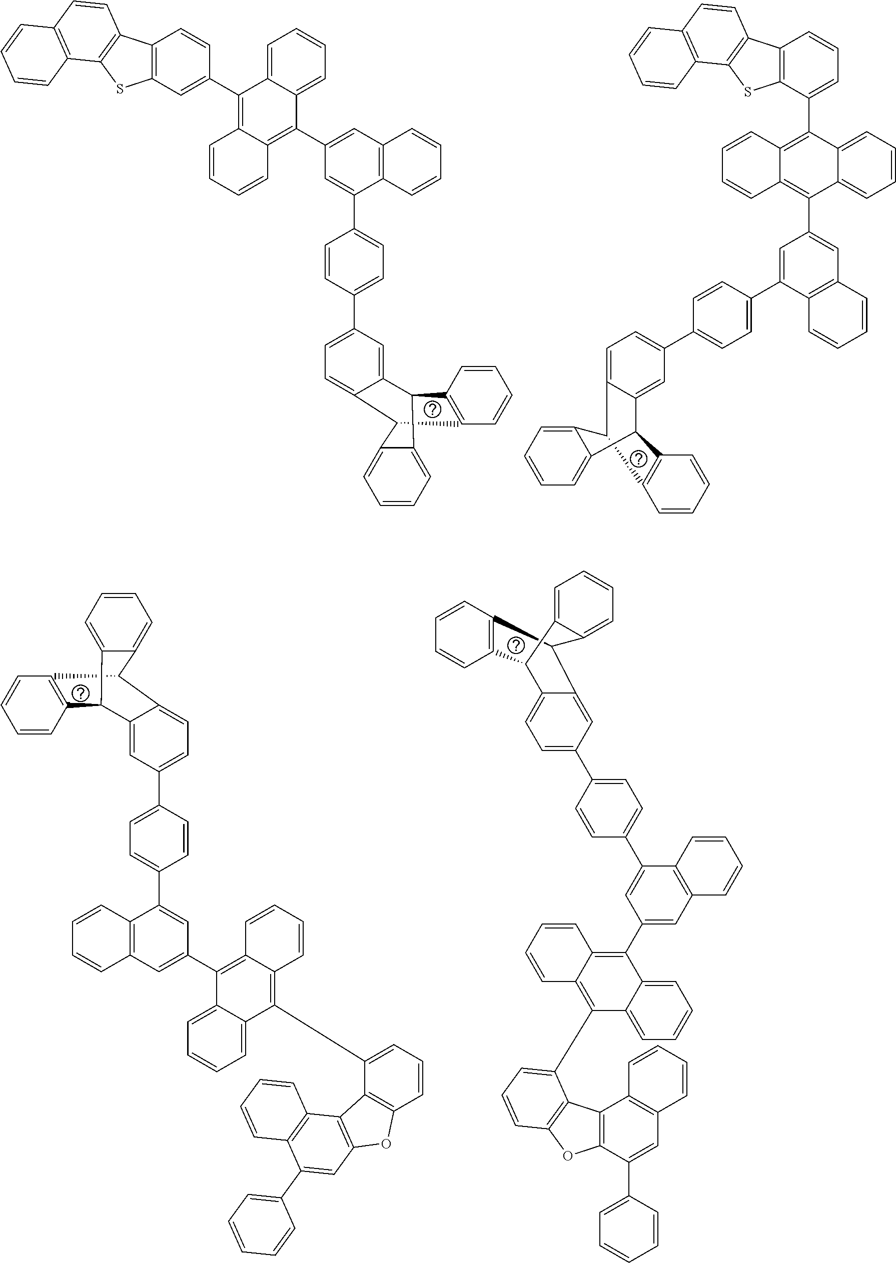

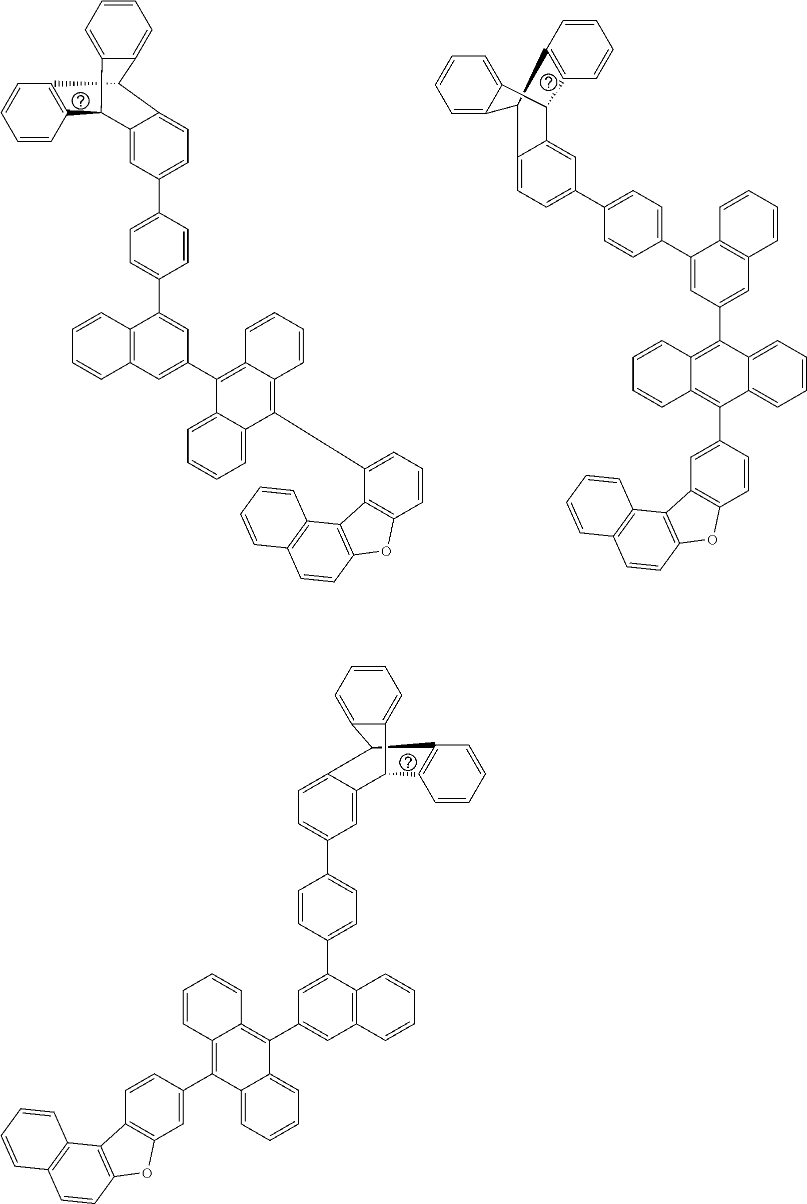 [SMILES: c1ccc2c(-c3ccc(-c4ccc5c(c4)[C@H]4c6ccccc6[C@@H]5c5ccccc54)cc3)cc(-c3c4ccccc4c(-c4cccc5oc6ccc7ccccc7c6c45)c4ccccc34)cc2c1 ; c1ccc2c(-c3ccc(-c4ccc5c(c4)[C@H]4c6ccccc6[C@@H]5c5ccccc54)cc3)cc(-c3c4ccccc4c(-c4ccc5oc6ccc7ccccc7c6c5c4)c4ccccc34)cc2c1 ; c1ccc2c(-c3ccc(-c4ccc5c(c4)[C@H]4c6ccccc6[C@@H]5c5ccccc54)cc3)cc(-c3c4ccccc4c(-c4ccc5c(c4)oc4ccc6ccccc6c45)c4ccccc34)cc2c1]

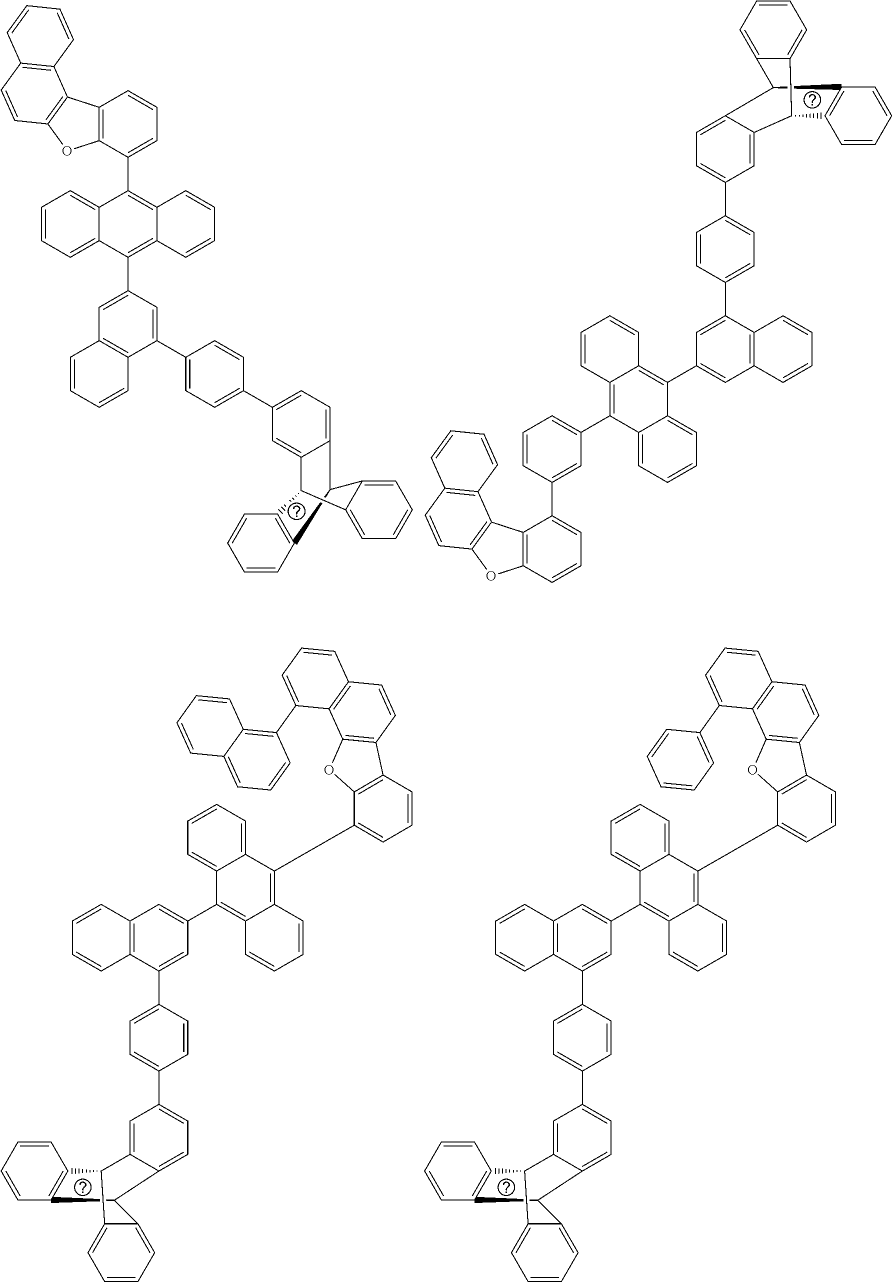

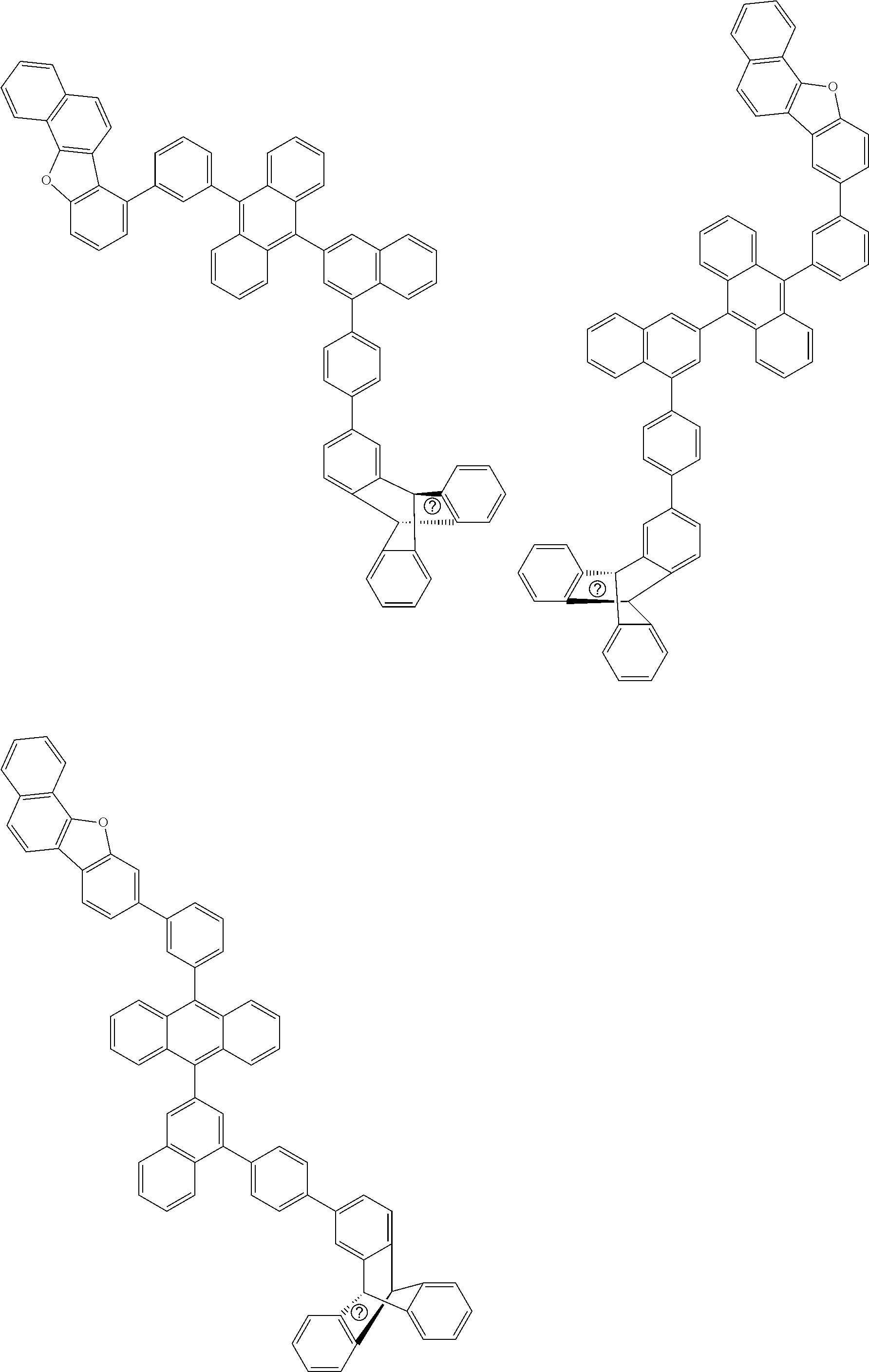 [SMILES: c1cc(-c2c3ccccc3c(-c3cc(-c4ccc(-c5ccc6c(c5)[C@H]5c7ccccc7[C@@H]6c6ccccc65)cc4)c4ccccc4c3)c3ccccc23)cc(-c2cccc3oc4c5ccccc5ccc4c23)c1 ; c1cc(-c2ccc3oc4c5ccccc5ccc4c3c2)cc(-c2c3ccccc3c(-c3cc(-c4ccc(-c5ccc6c(c5)[C@H]5c7ccccc7[C@@H]6c6ccccc65)cc4)c4ccccc4c3)c3ccccc23)c1 ; c1cc(-c2ccc3c(c2)oc2c4ccccc4ccc32)cc(-c2c3ccccc3c(-c3cc(-c4ccc(-c5ccc6c(c5)[C@H]5c7ccccc7[C@@H]6c6ccccc65)cc4)c4ccccc4c3)c3ccccc23)c1]

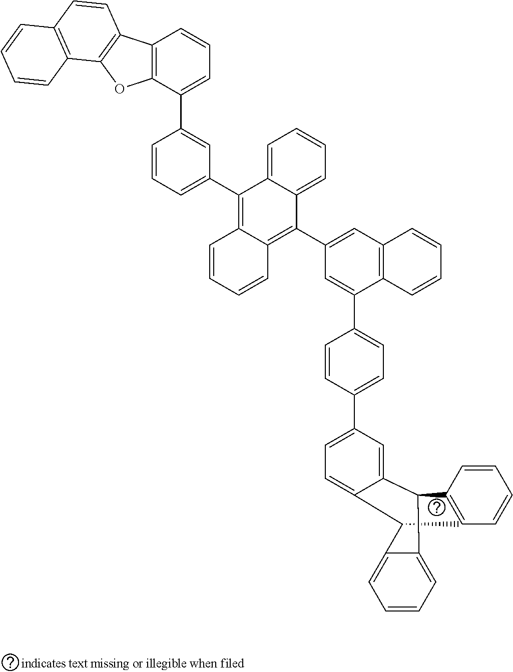 [SMILES: c1cc(-c2c3ccccc3c(-c3cc(-c4ccc(-c5ccc6c(c5)[C@H]5c7ccccc7[C@@H]6c6ccccc65)cc4)c4ccccc4c3)c3ccccc23)cc(-c2cccc3c2oc2c4ccccc4ccc32)c1]

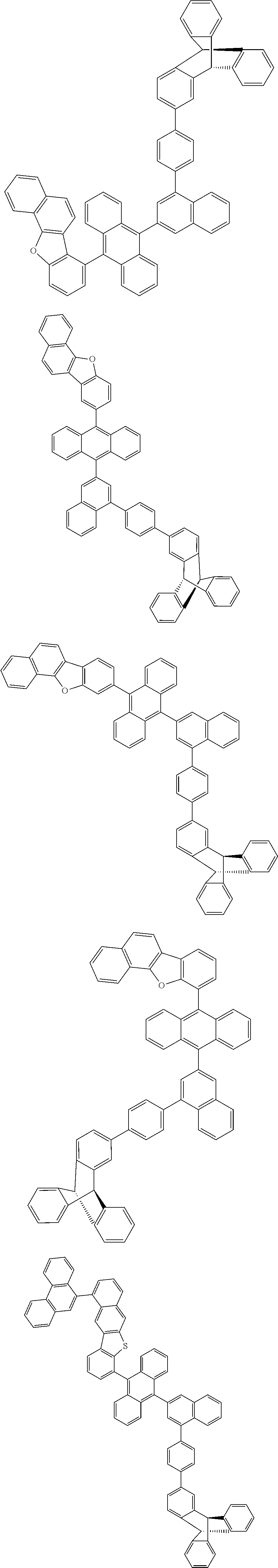

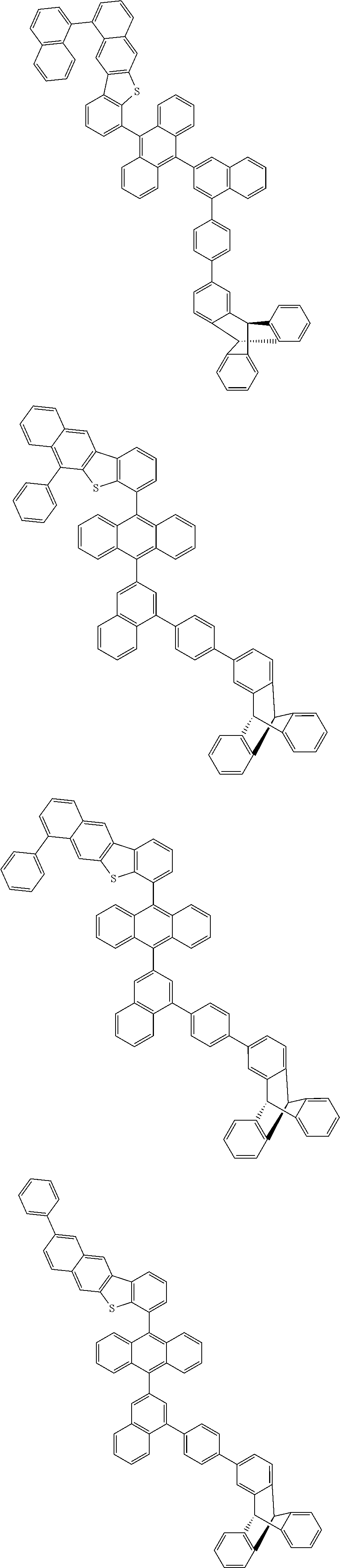

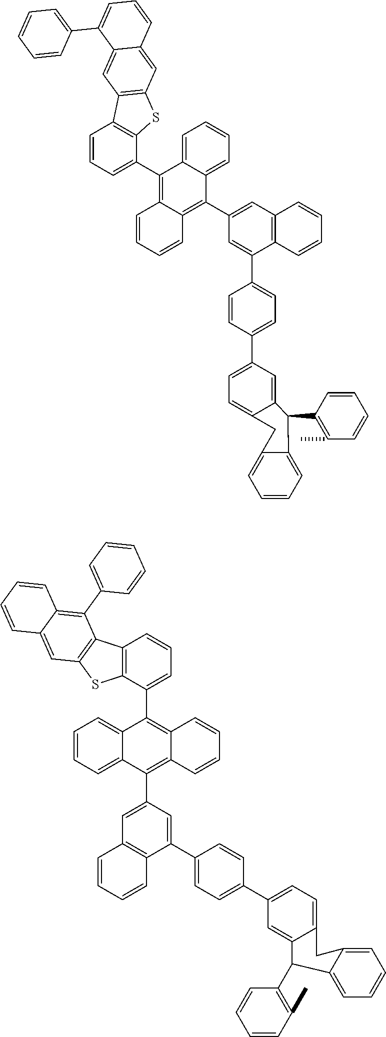

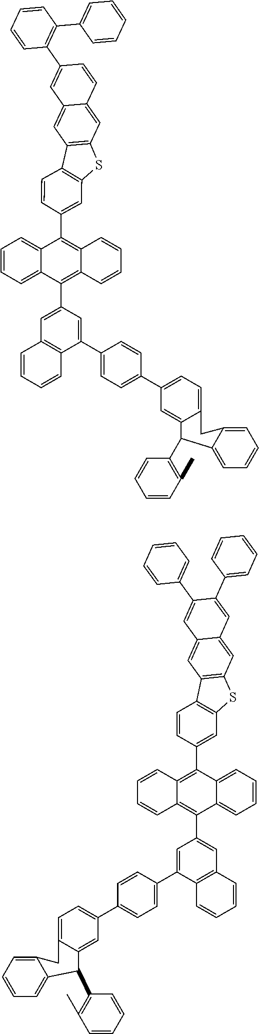

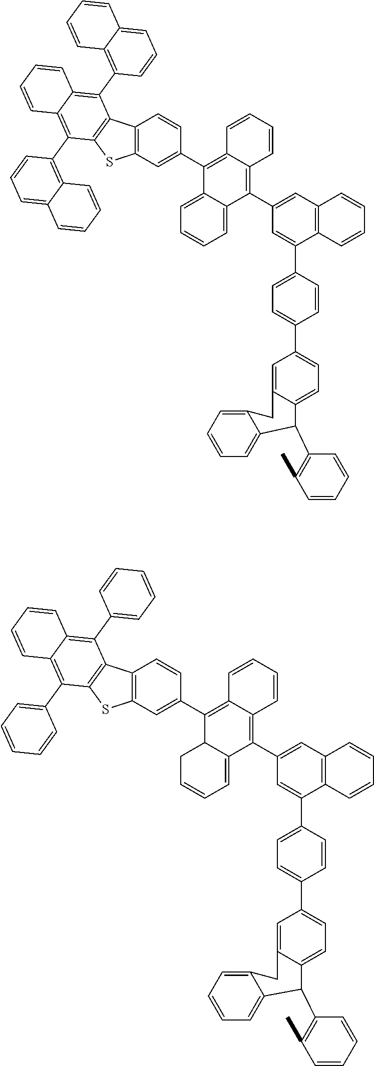

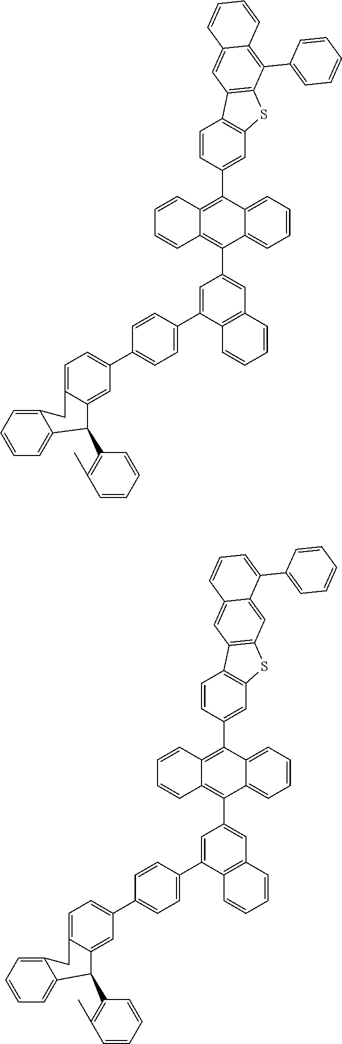

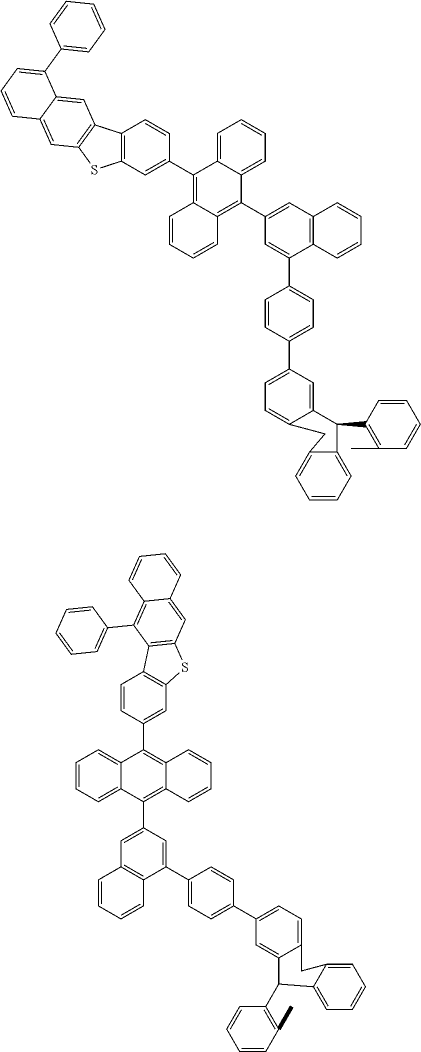

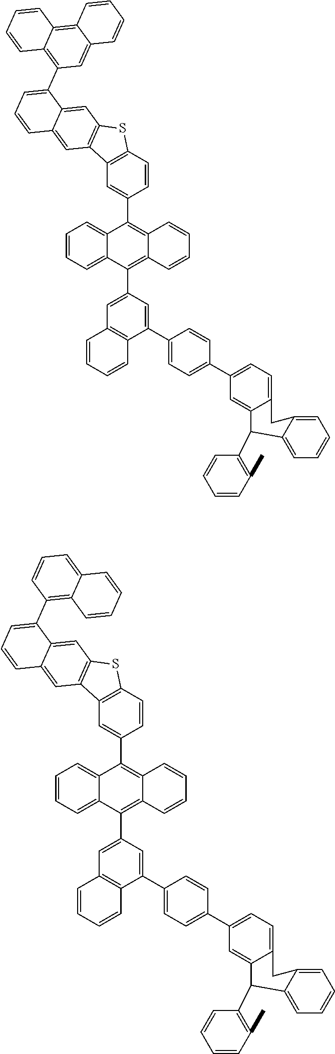

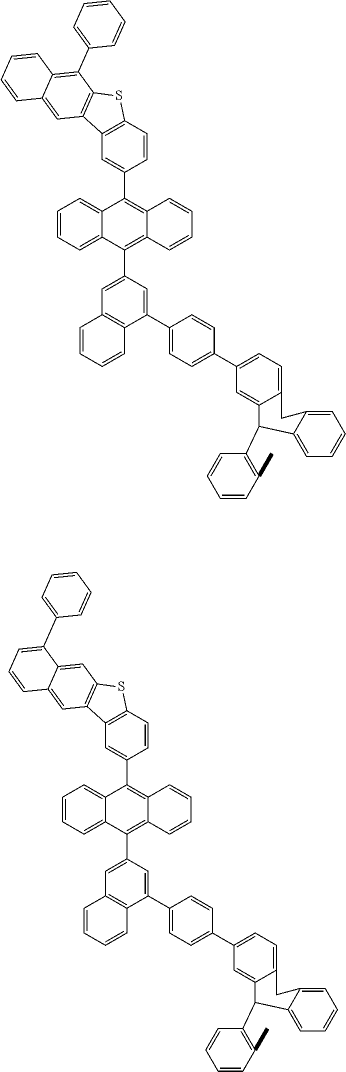

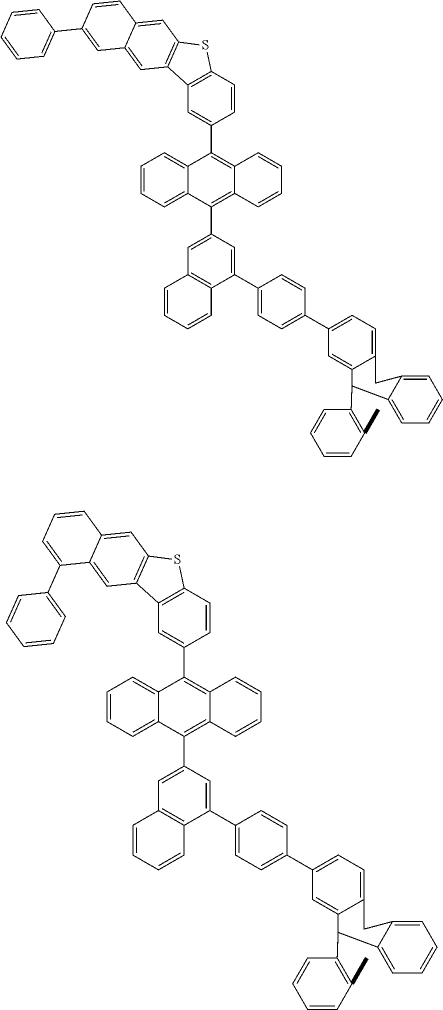

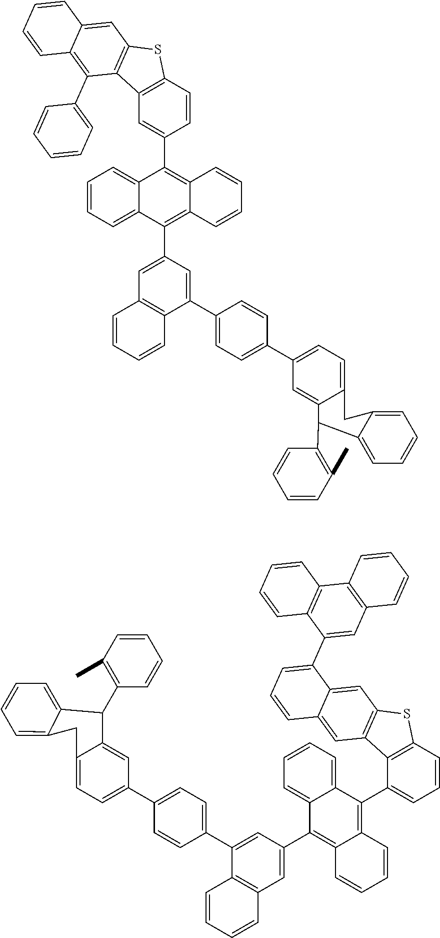

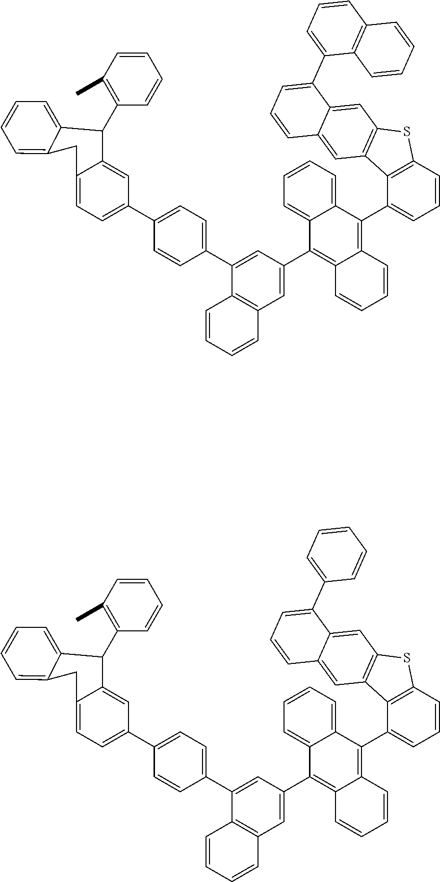

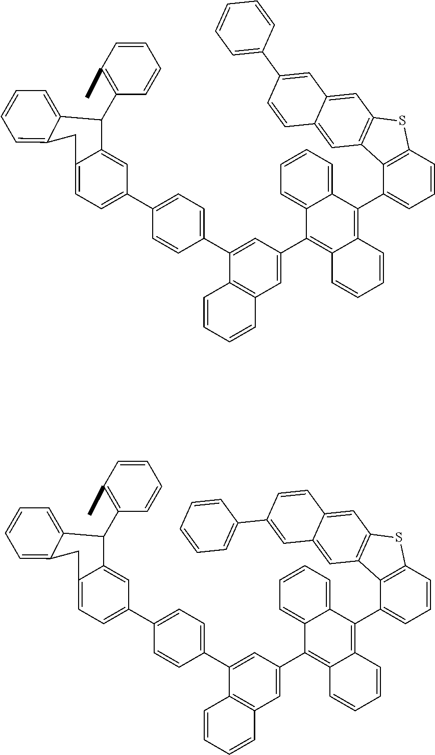

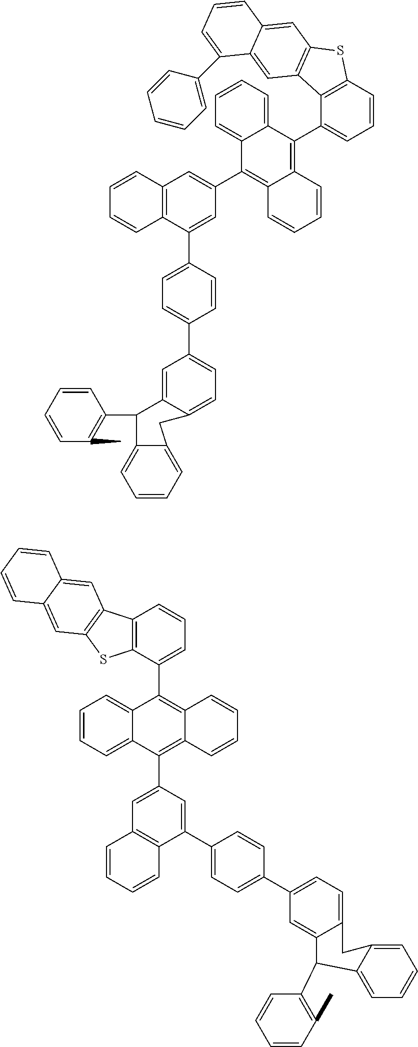

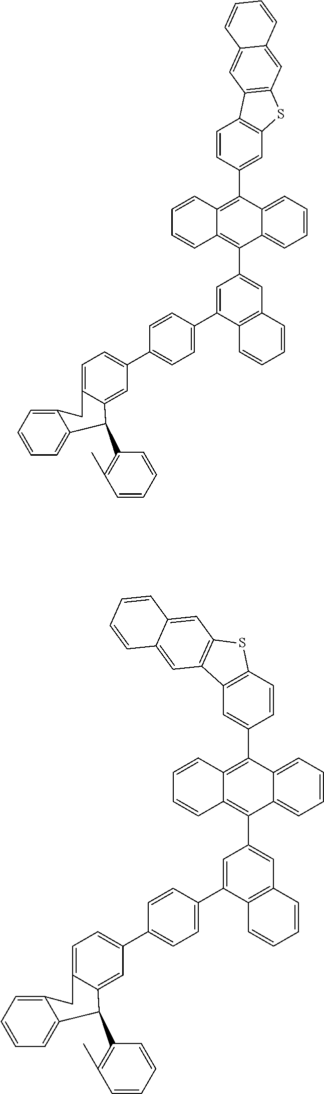

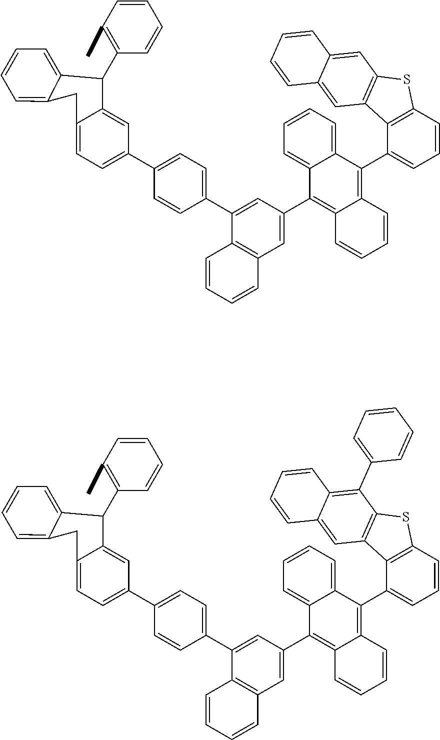

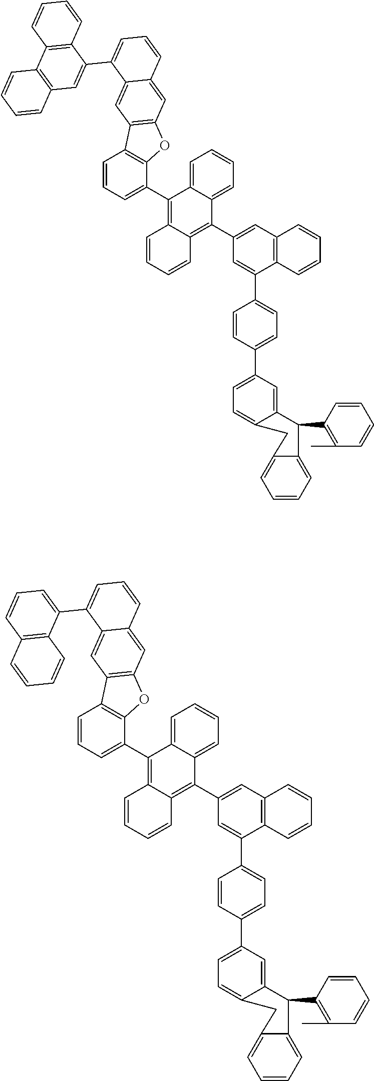

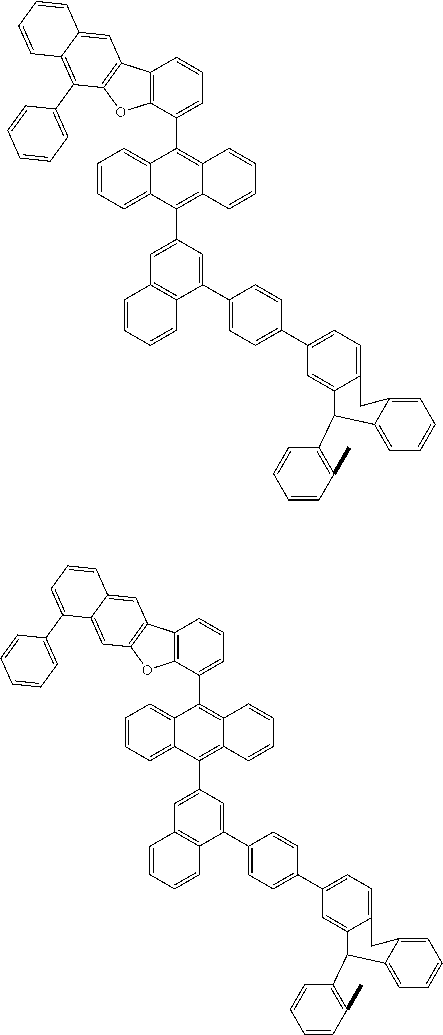

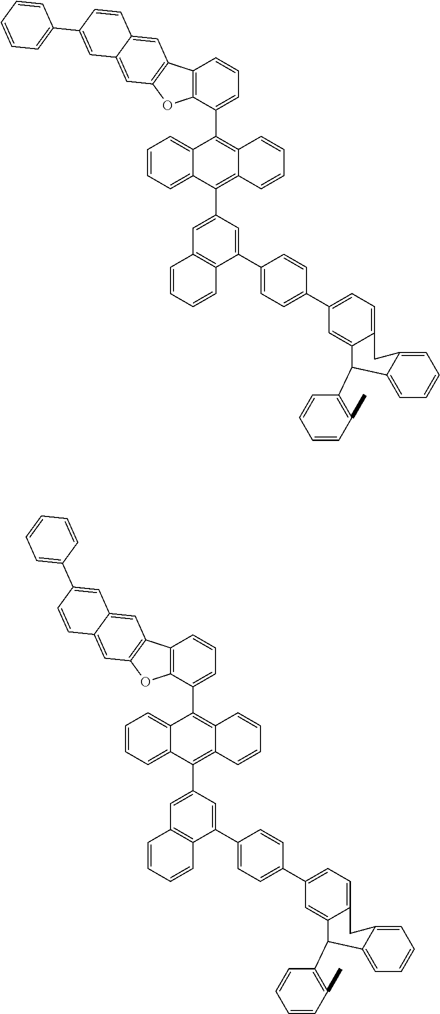

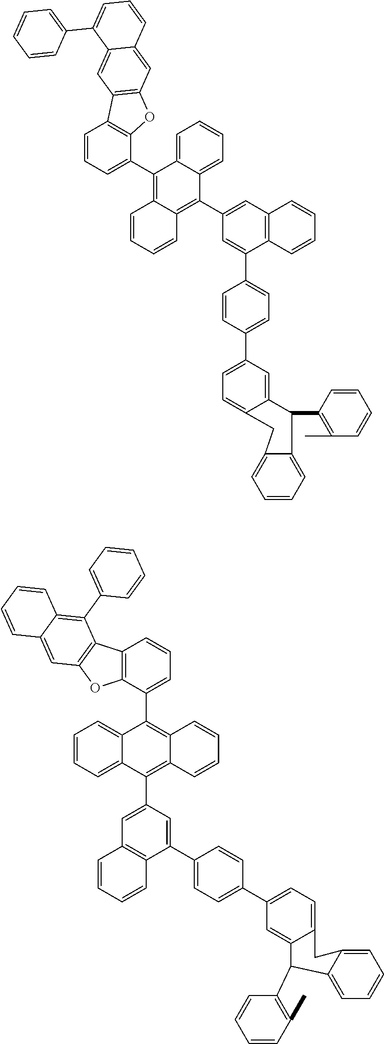

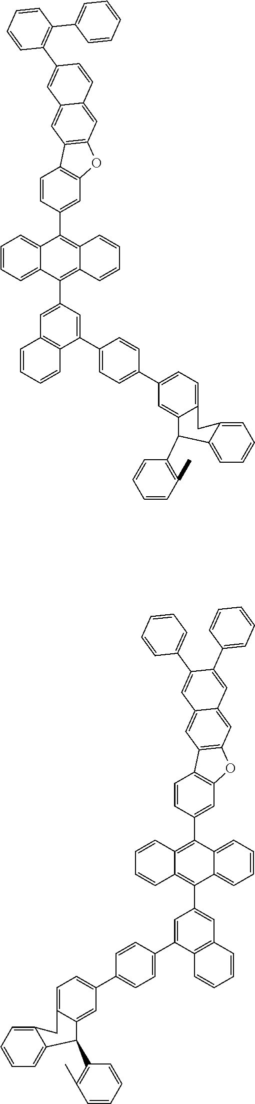

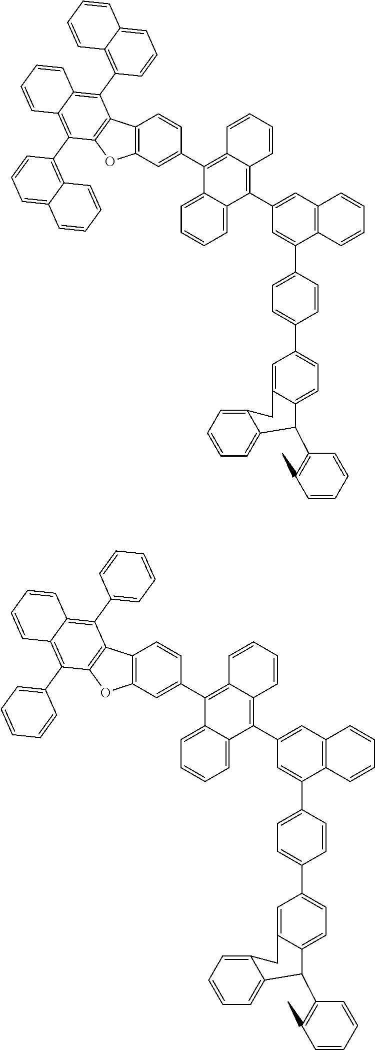

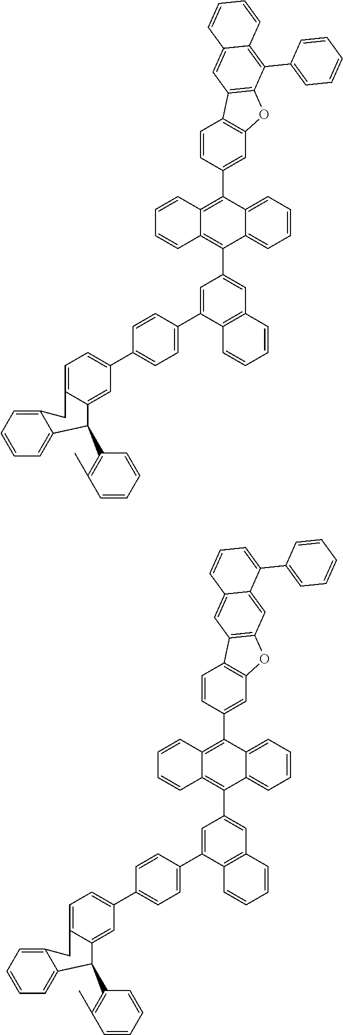

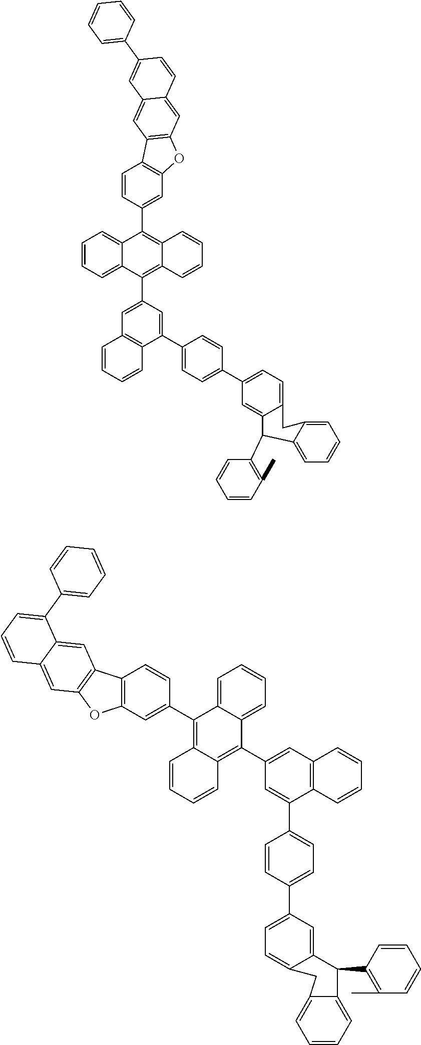

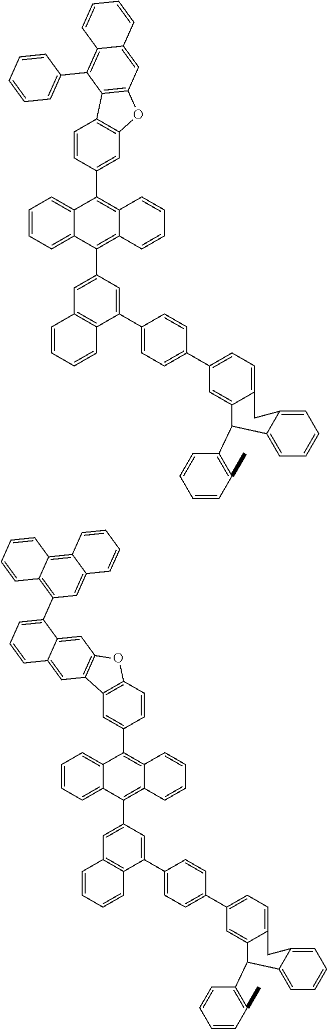

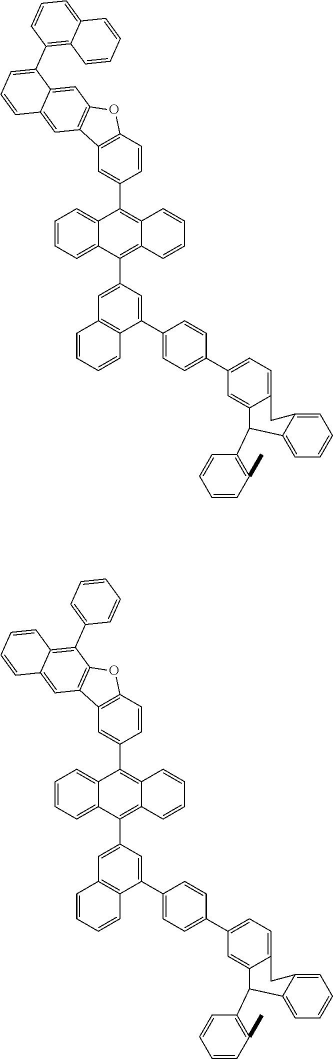

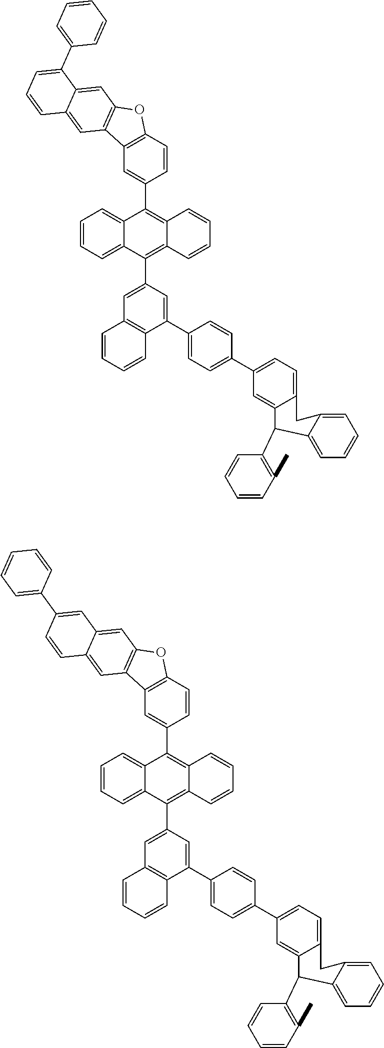

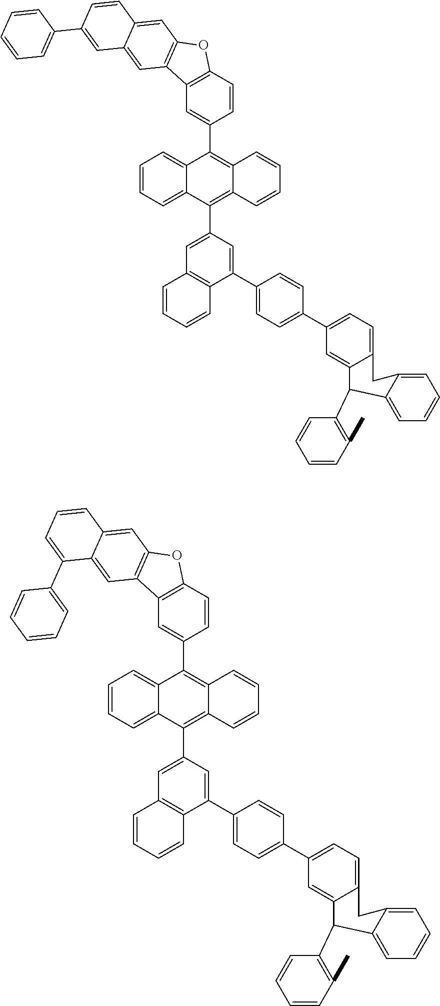

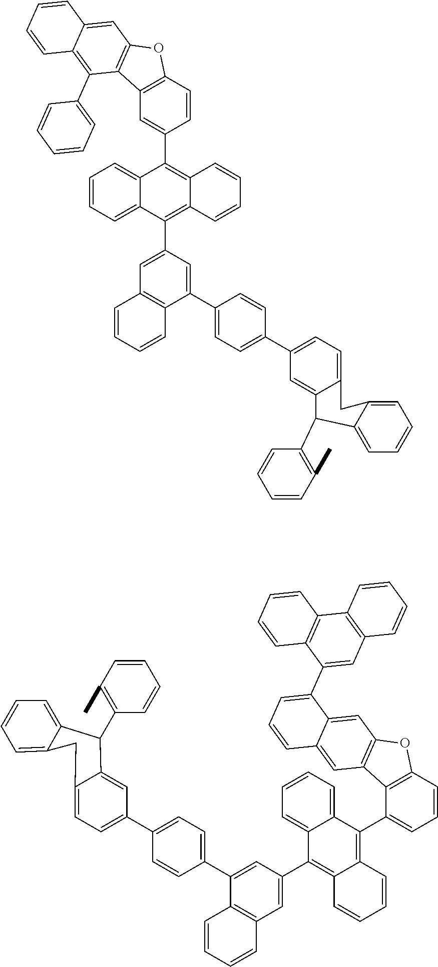

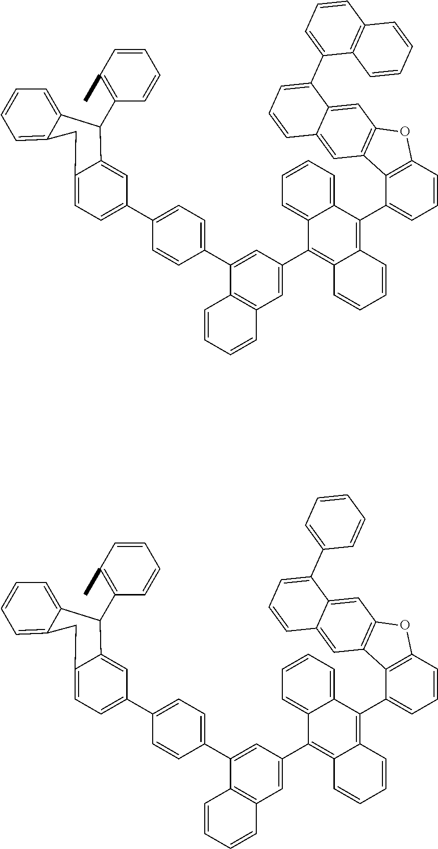

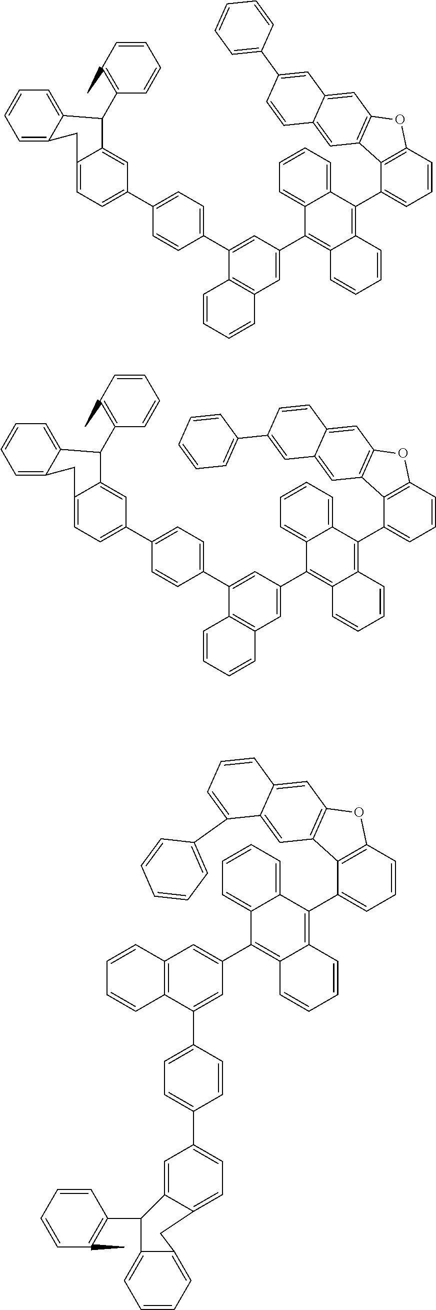

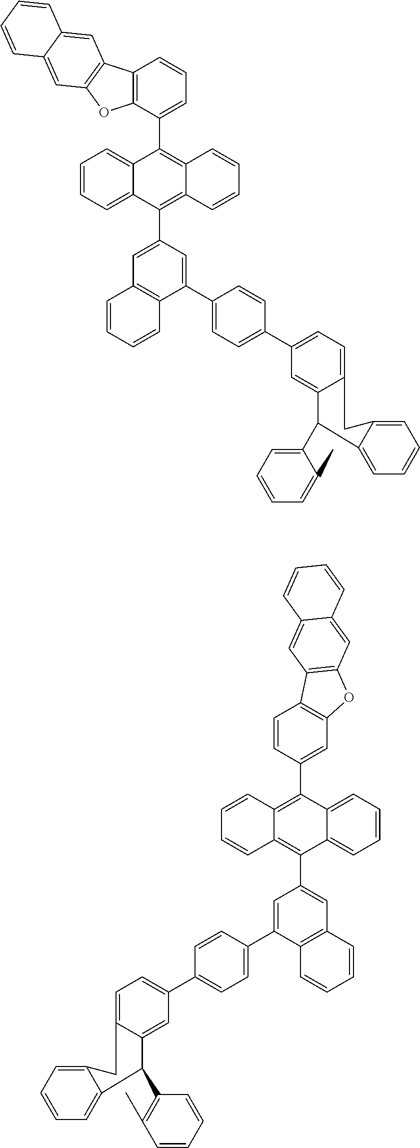

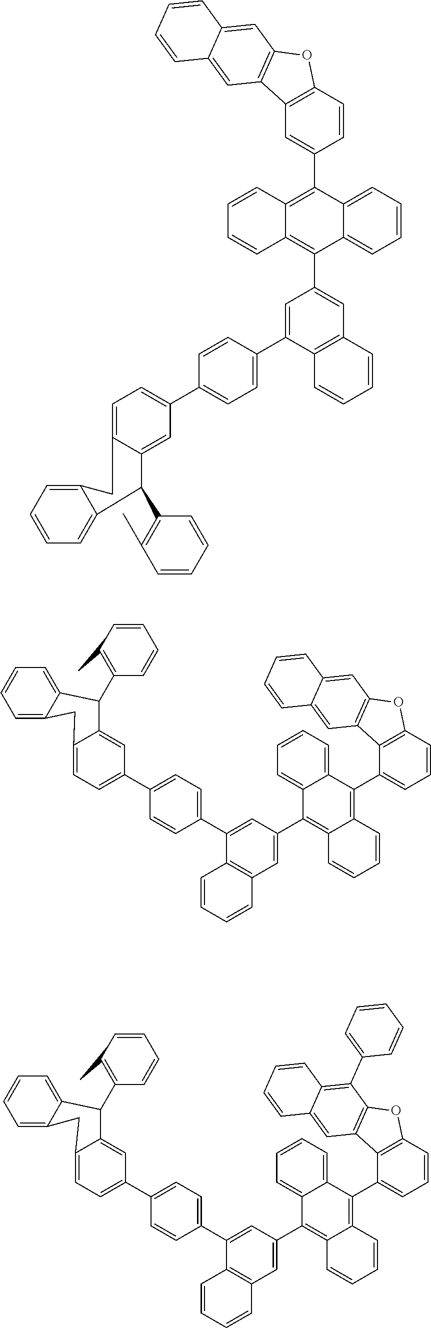

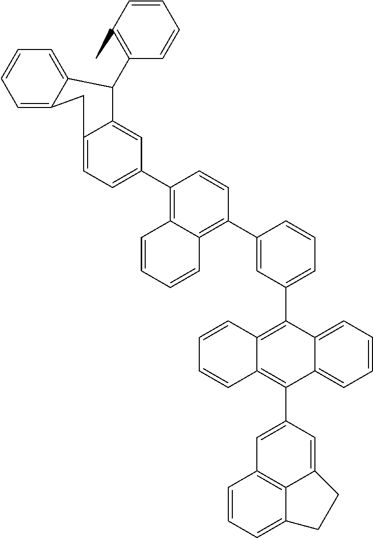

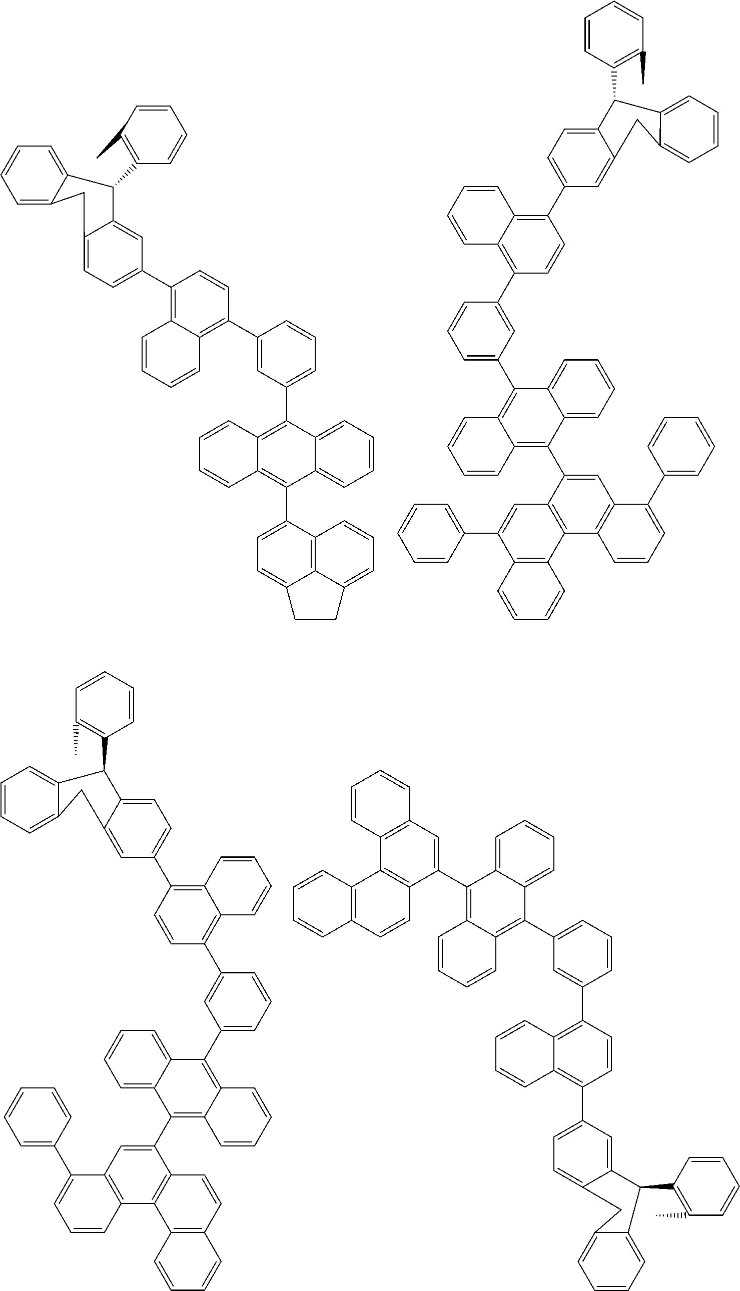

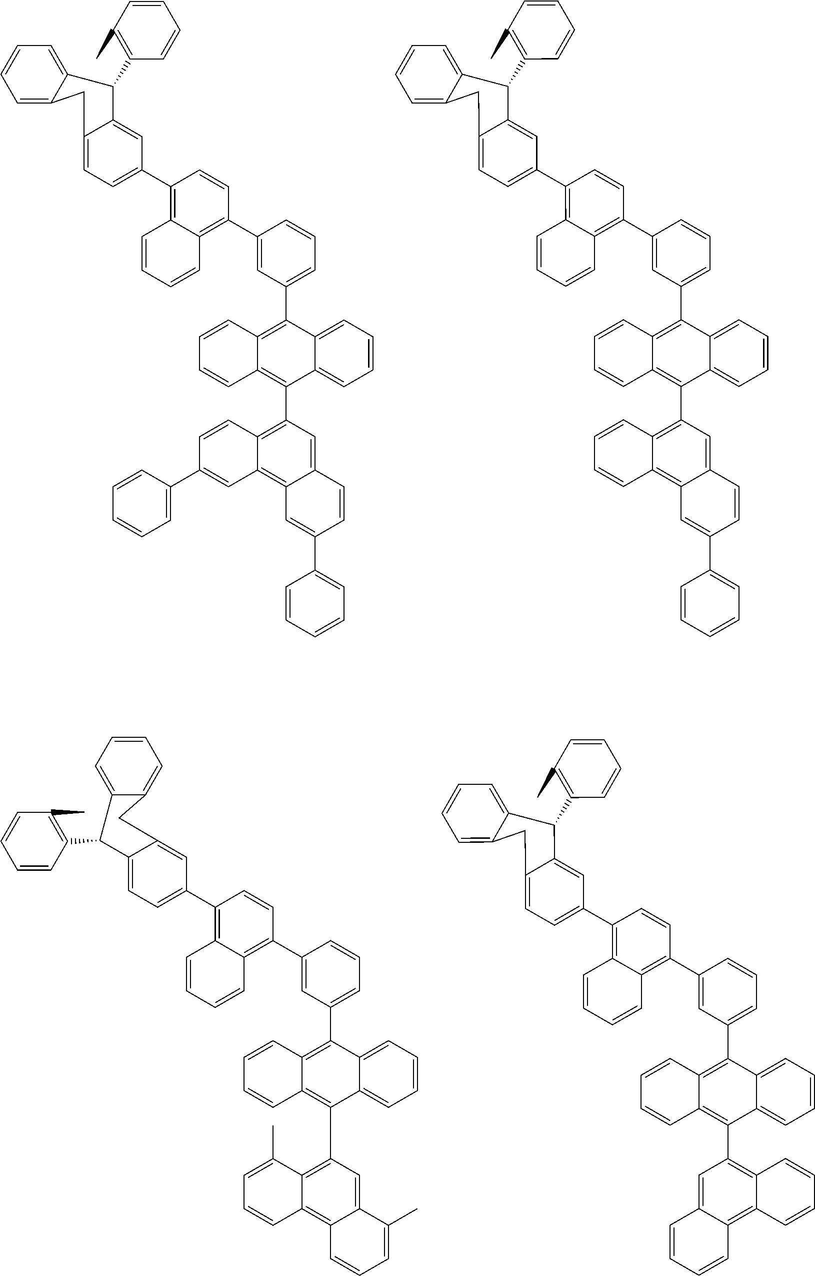

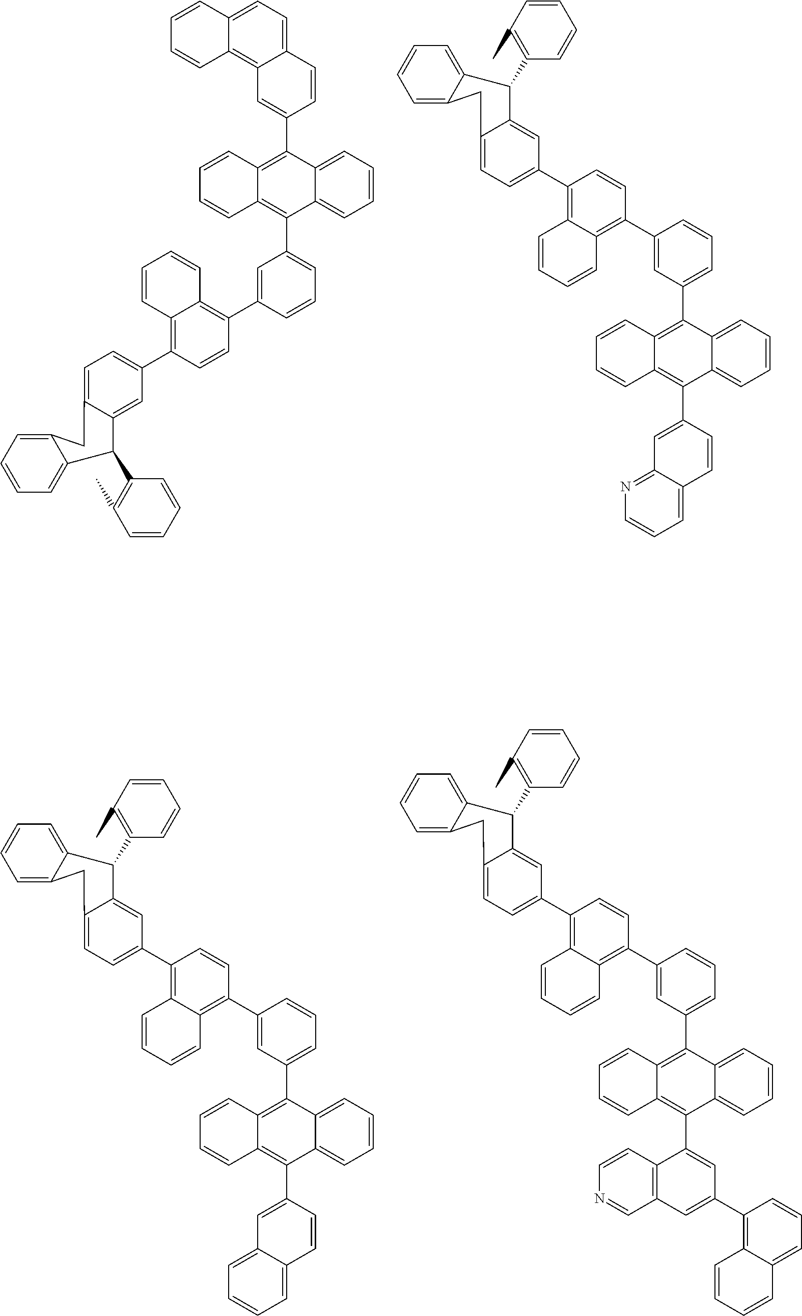

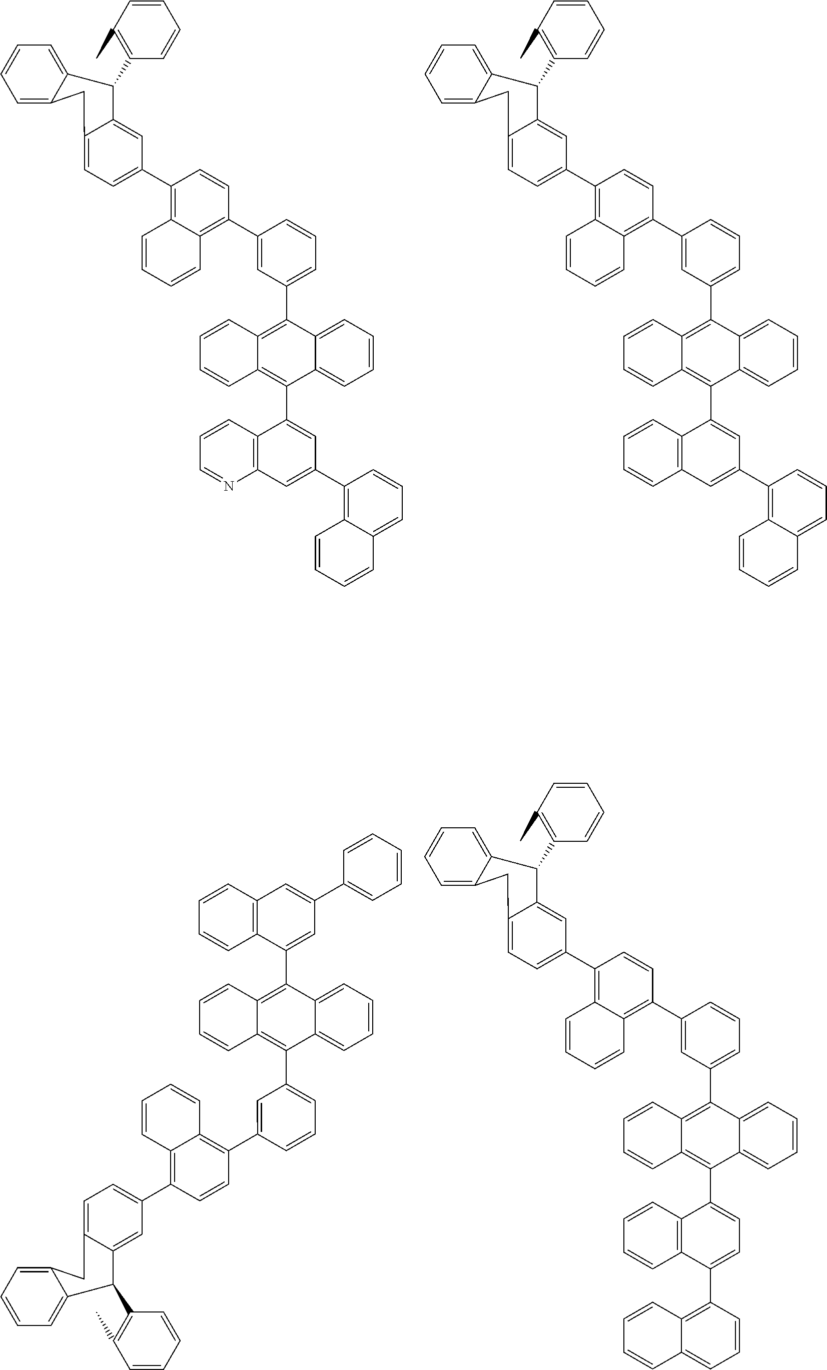

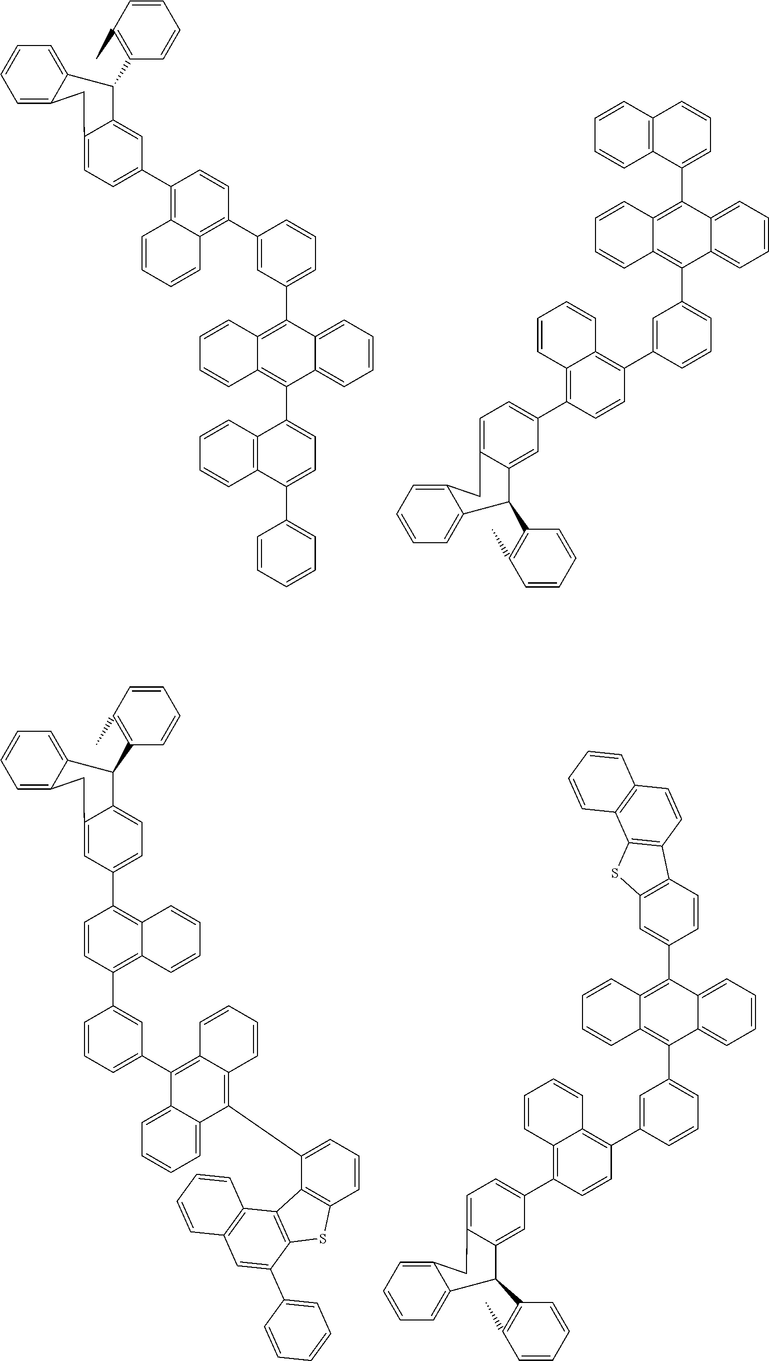

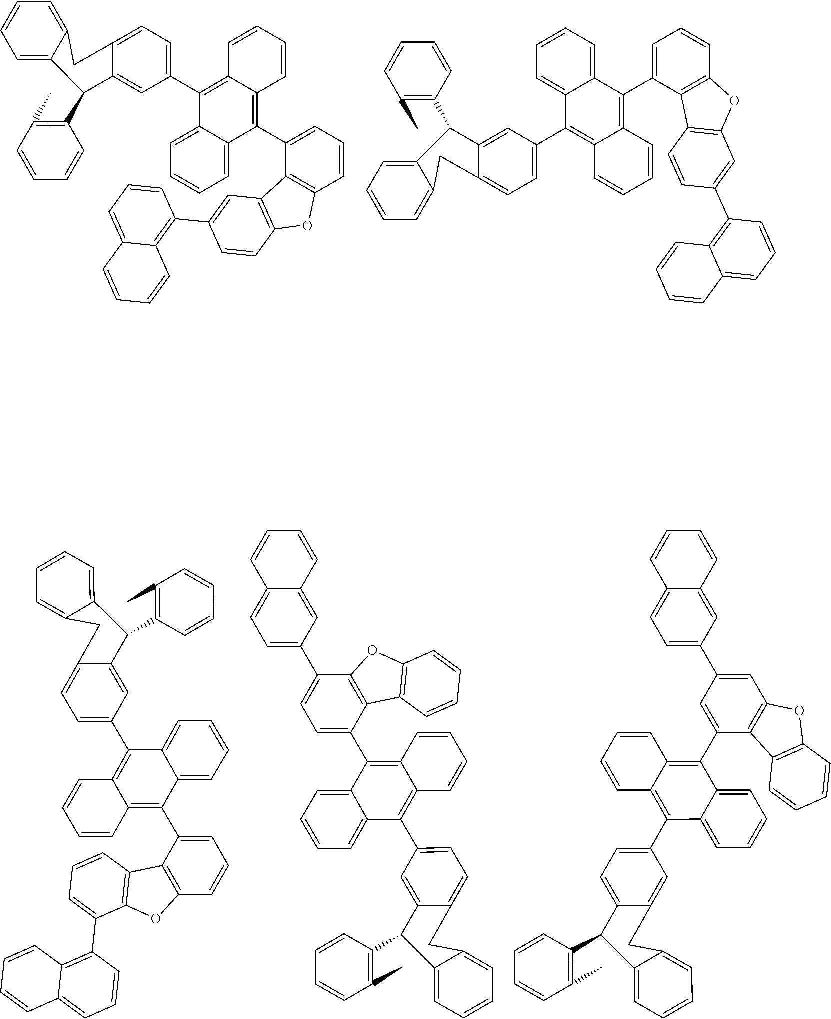

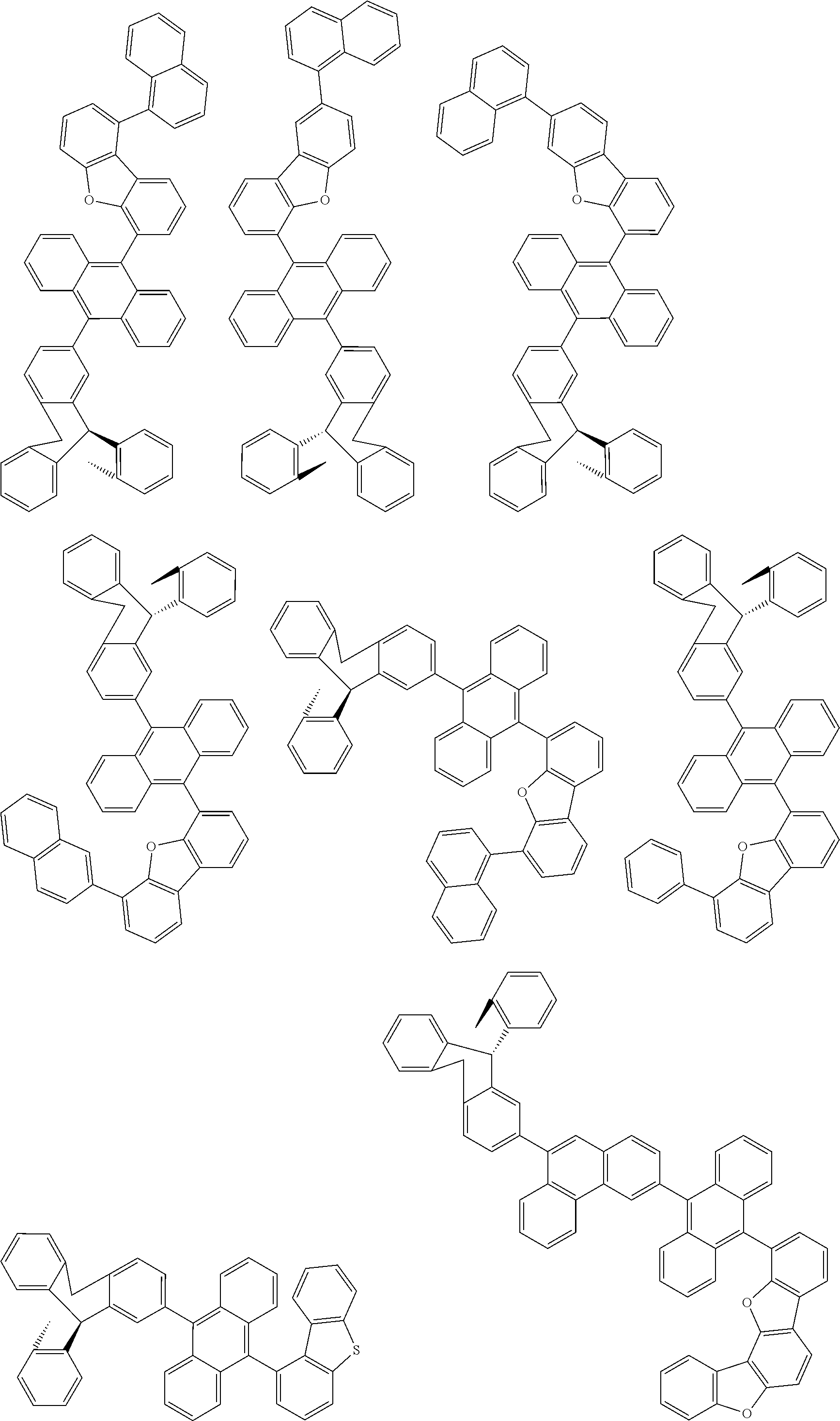

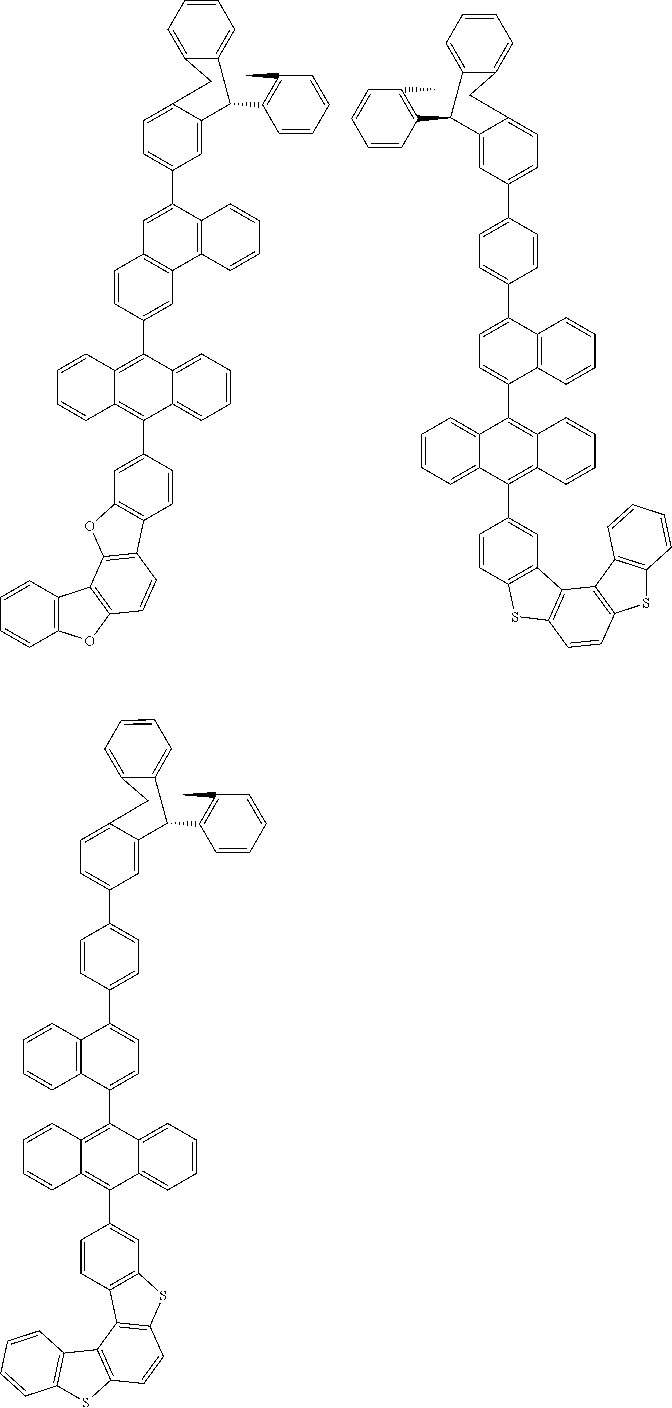

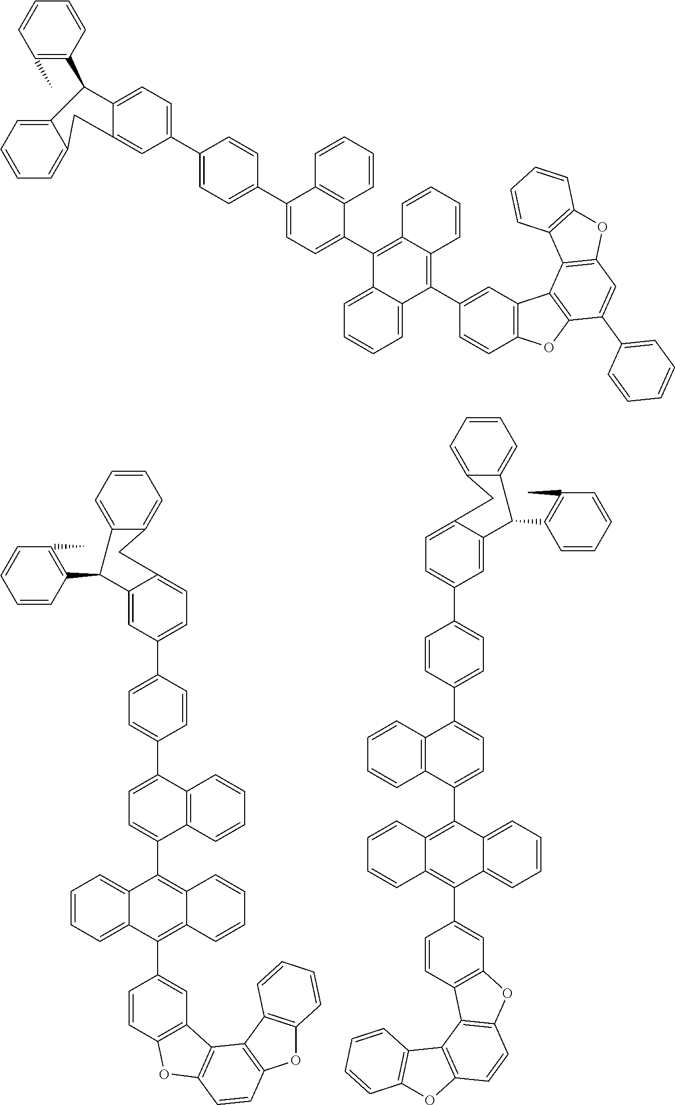

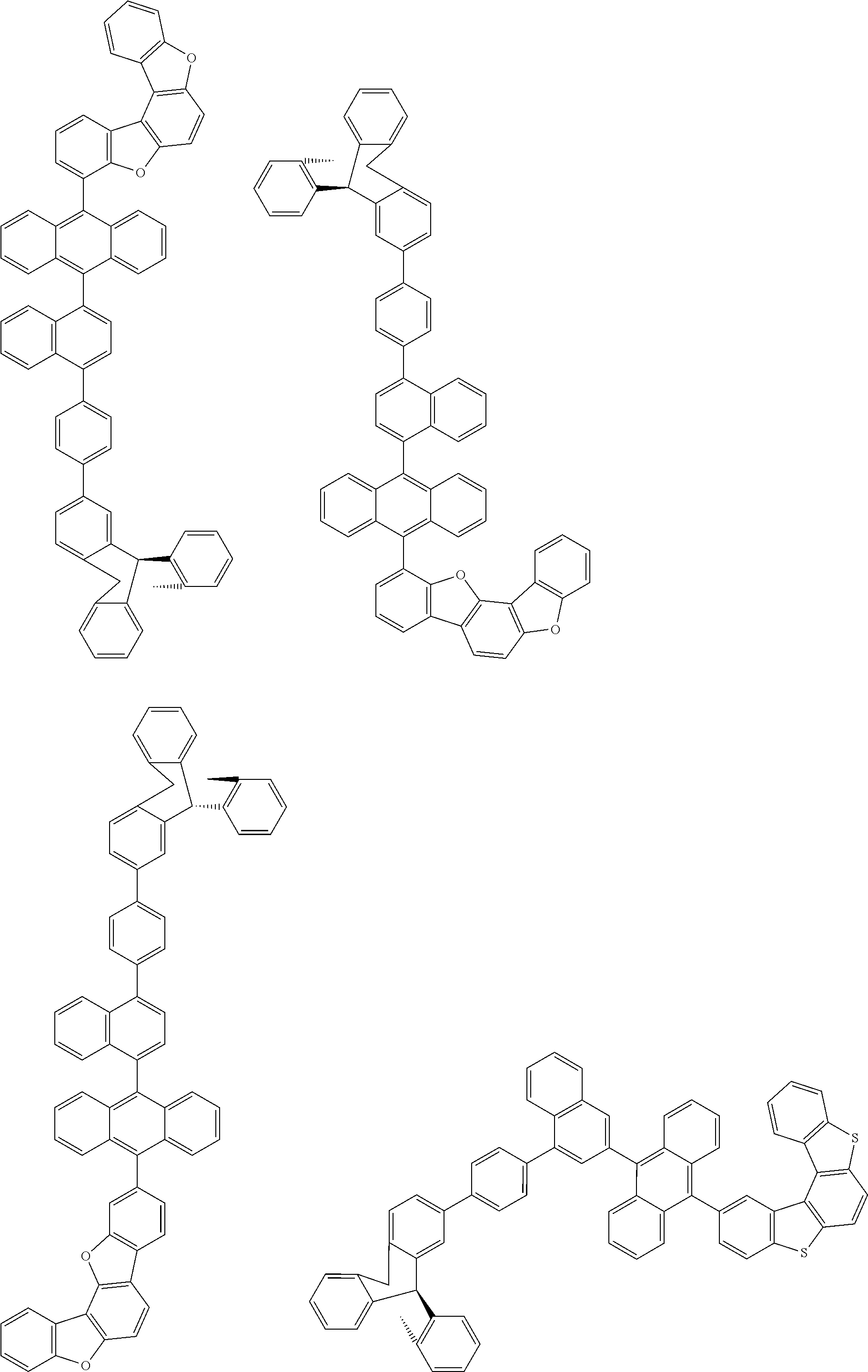

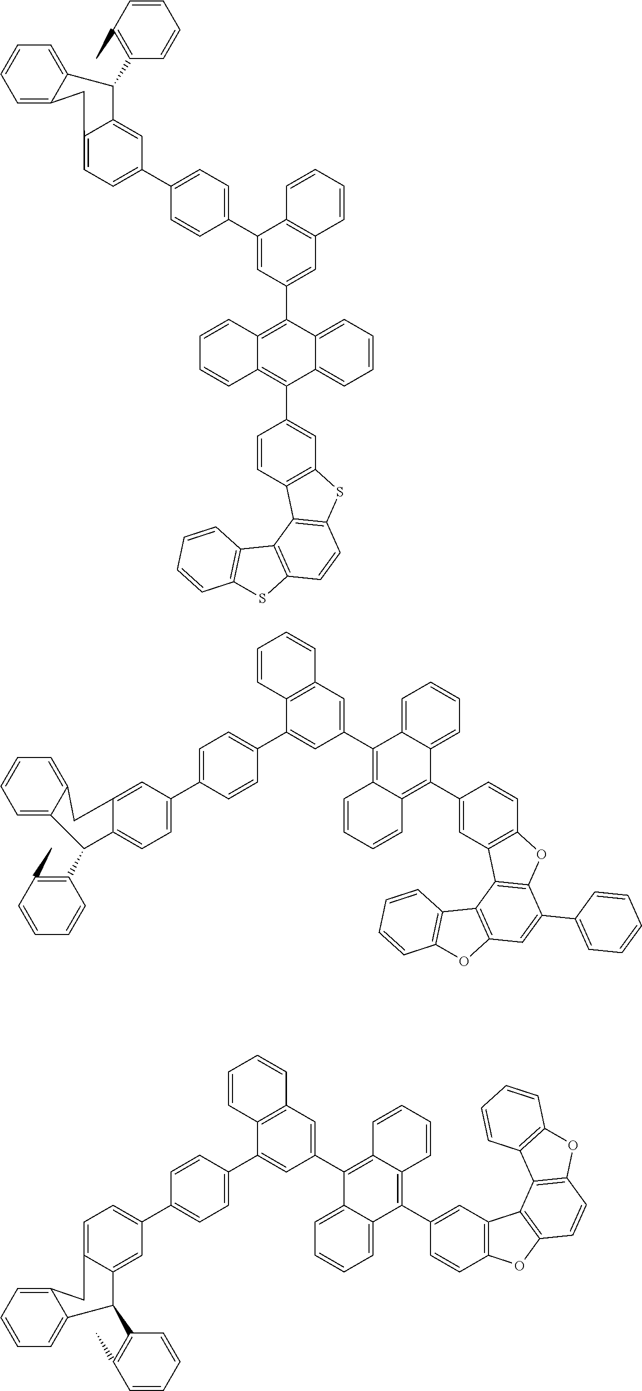

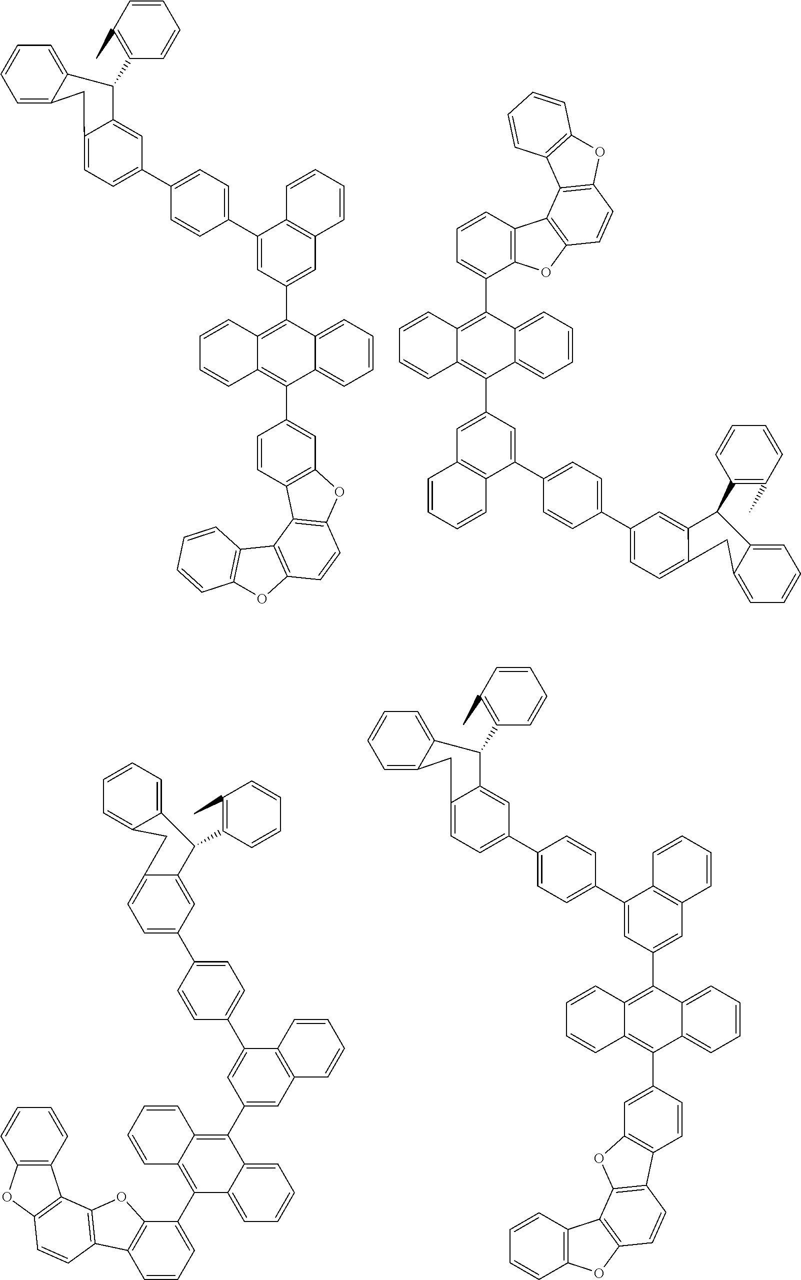

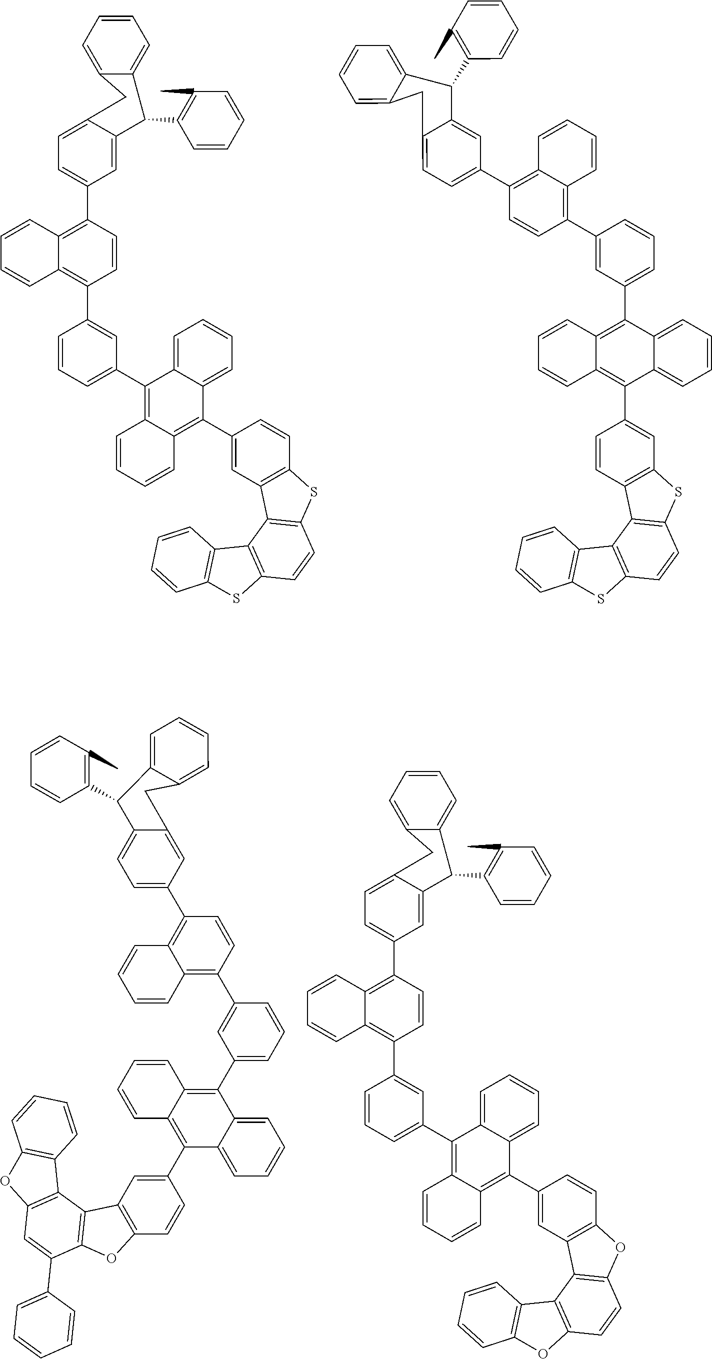

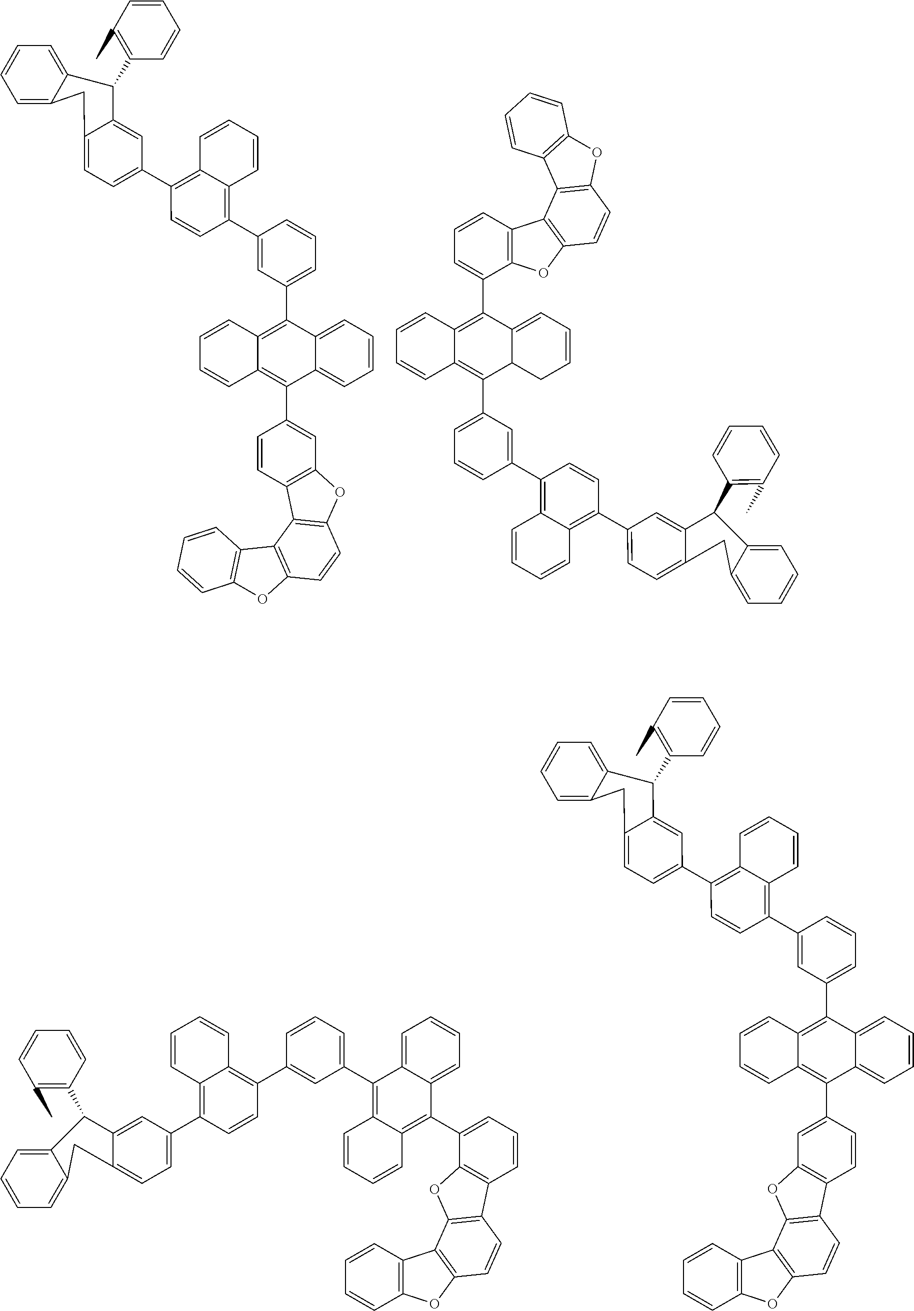

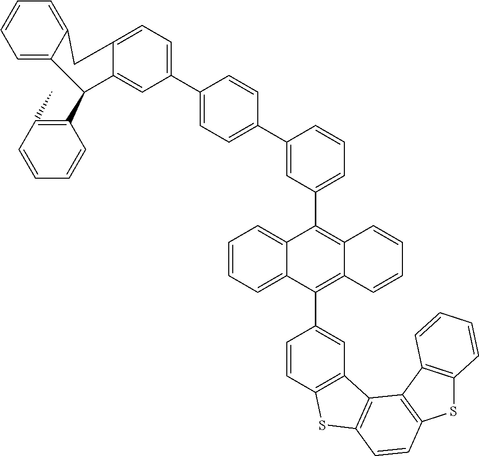

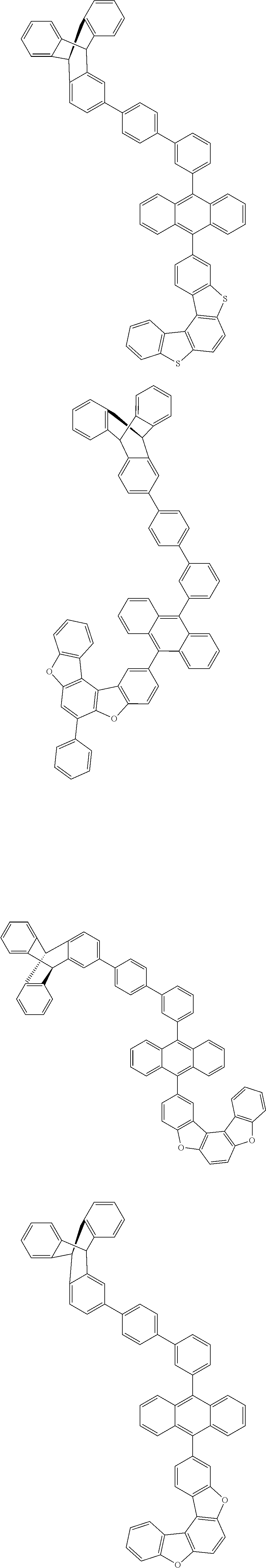

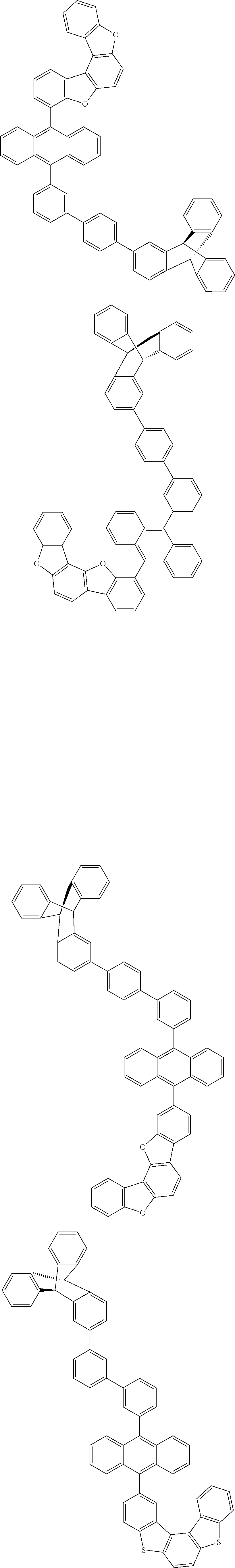

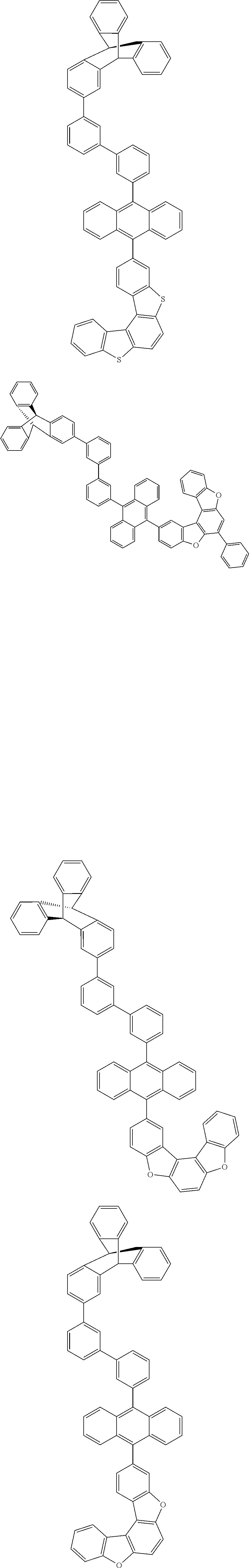

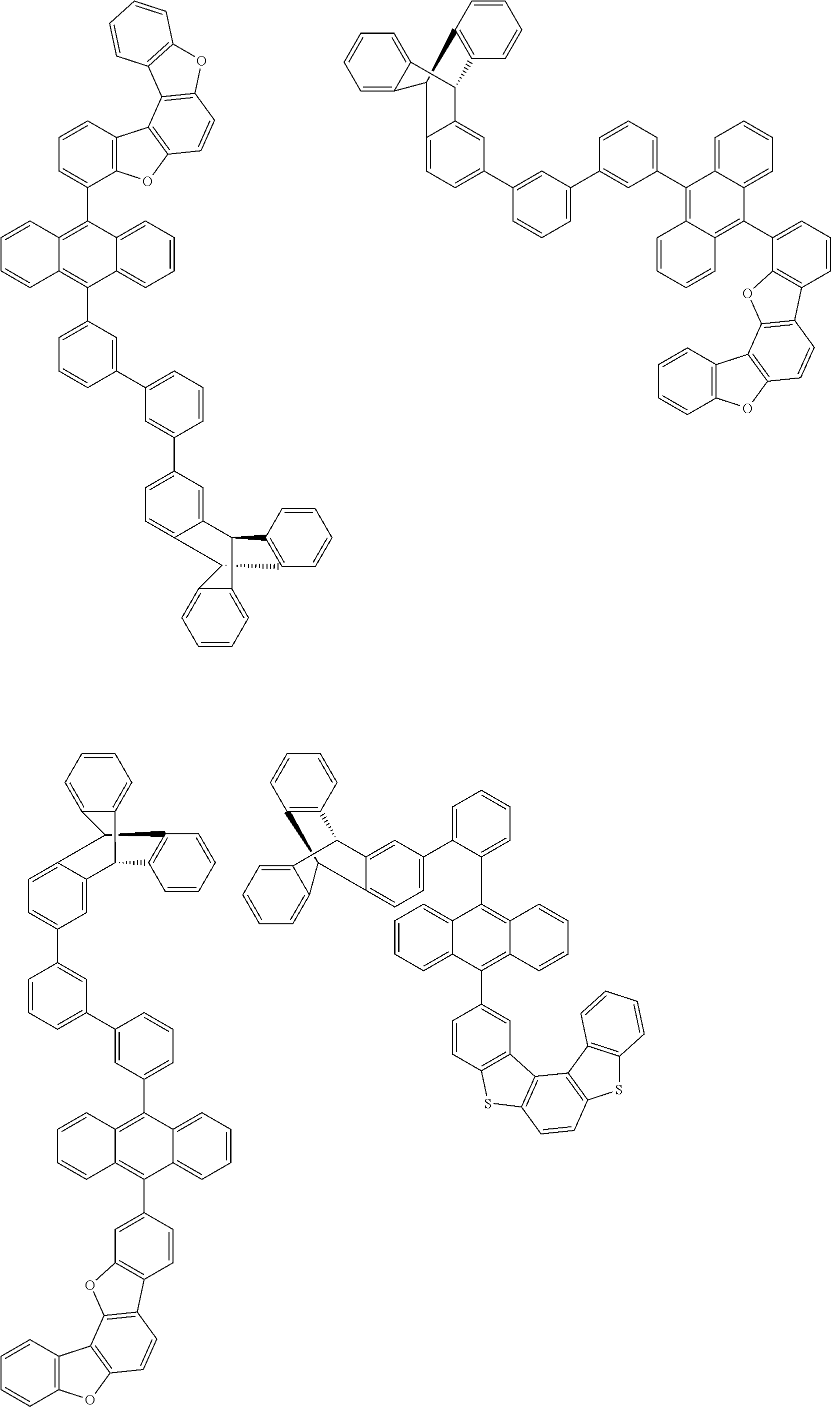

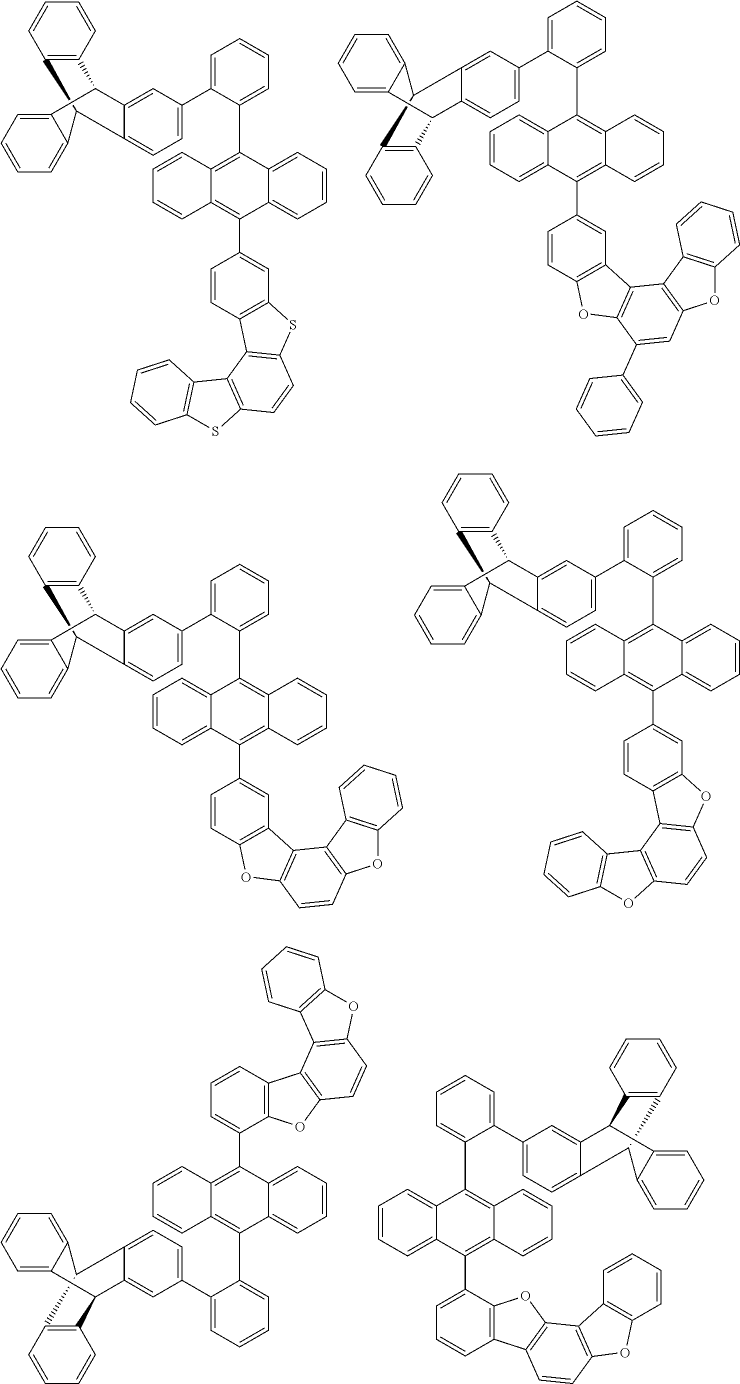

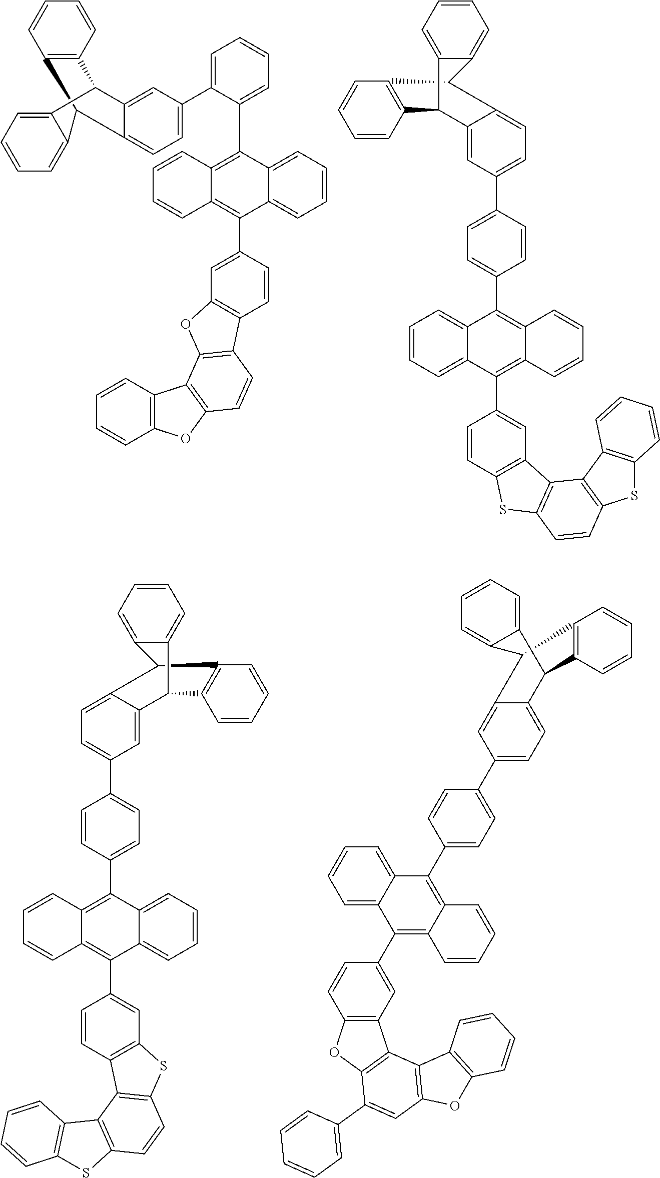

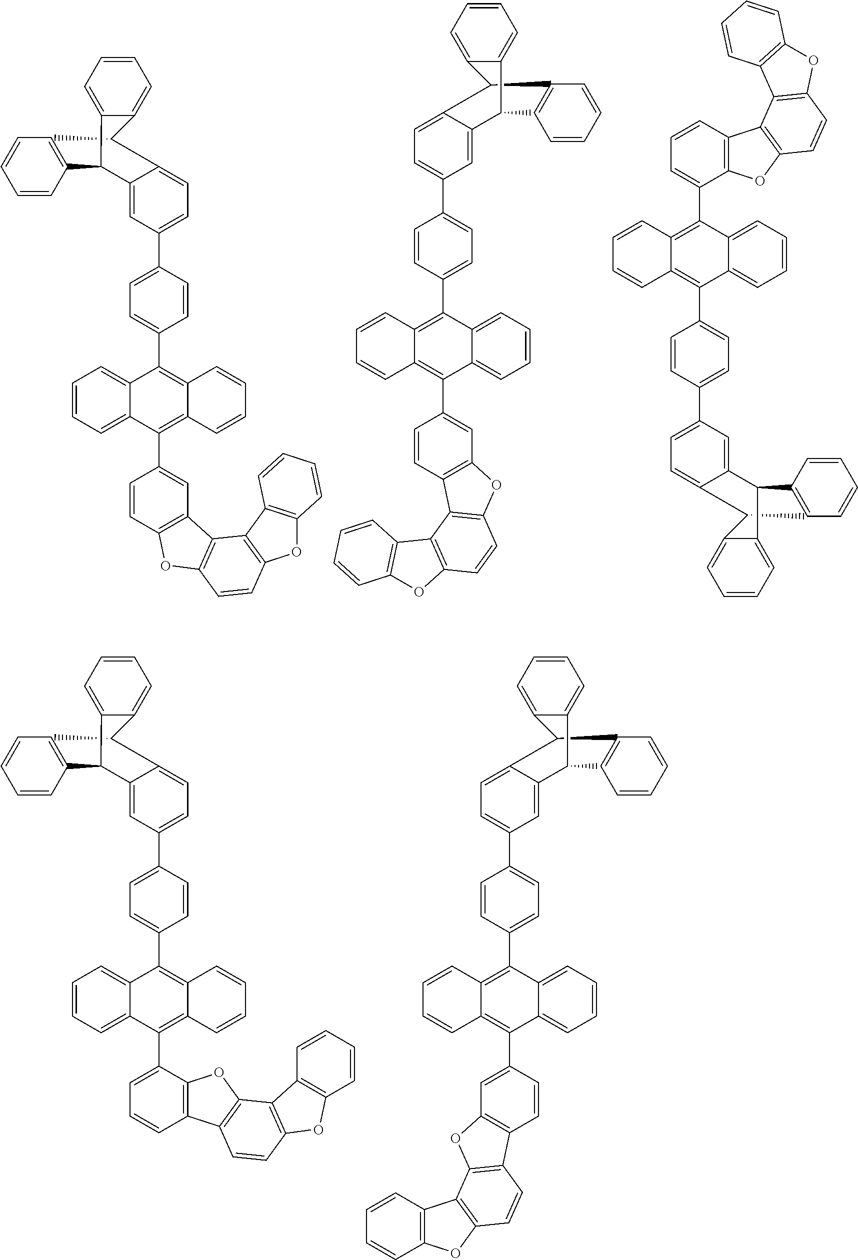

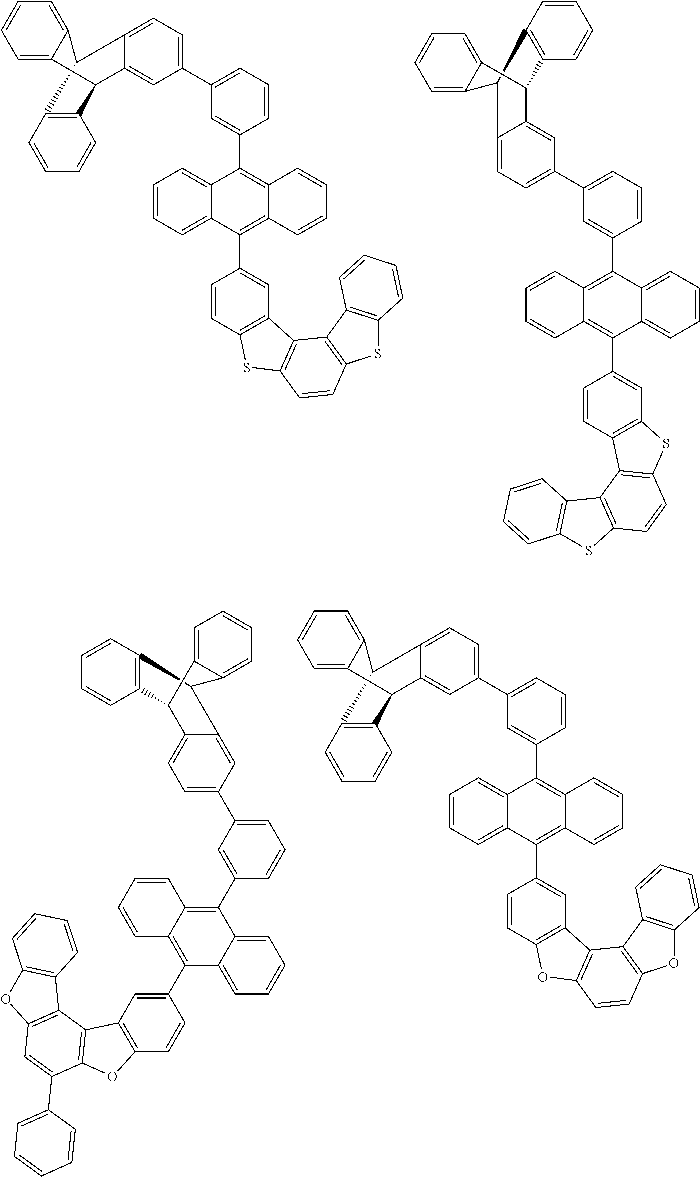 [SMILES: c1cc(-c2ccc3c(c2)[C@H]2c4ccccc4[C@@H]3c3ccccc32)cc(-c2c3ccccc3c(-c3ccc4sc5ccc6sc7ccccc7c6c5c4c3)c3ccccc23)c1 ; c1cc(-c2ccc3c(c2)[C@H]2c4ccccc4[C@@H]3c3ccccc32)cc(-c2c3ccccc3c(-c3ccc4c(c3)sc3ccc5sc6ccccc6c5c34)c3ccccc23)c1 ; c1ccc(-c2cc3oc4ccccc4c3c3c2oc2ccc(-c4c5ccccc5c(-c5cccc(-c6ccc7c(c6)[C@H]6c8ccccc8[C@@H]7c7ccccc76)c5)c5ccccc45)cc23)cc1 ; c1cc(-c2ccc3c(c2)[C@H]2c4ccccc4[C@@H]3c3ccccc32)cc(-c2c3ccccc3c(-c3ccc4oc5ccc6oc7ccccc7c6c5c4c3)c3ccccc23)c1]

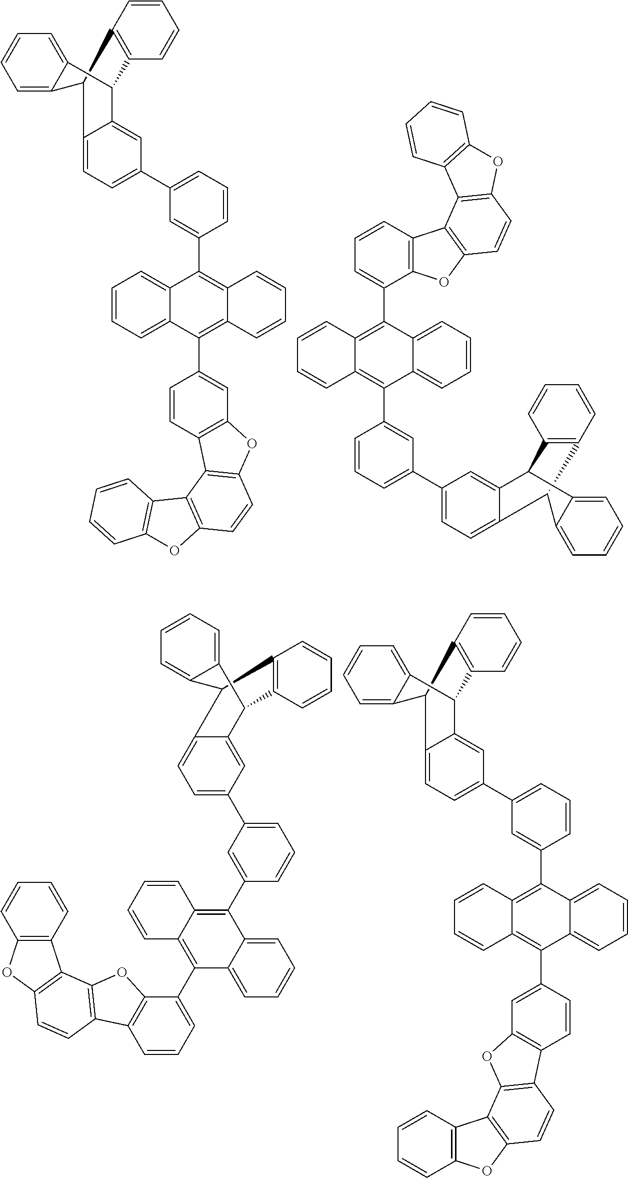

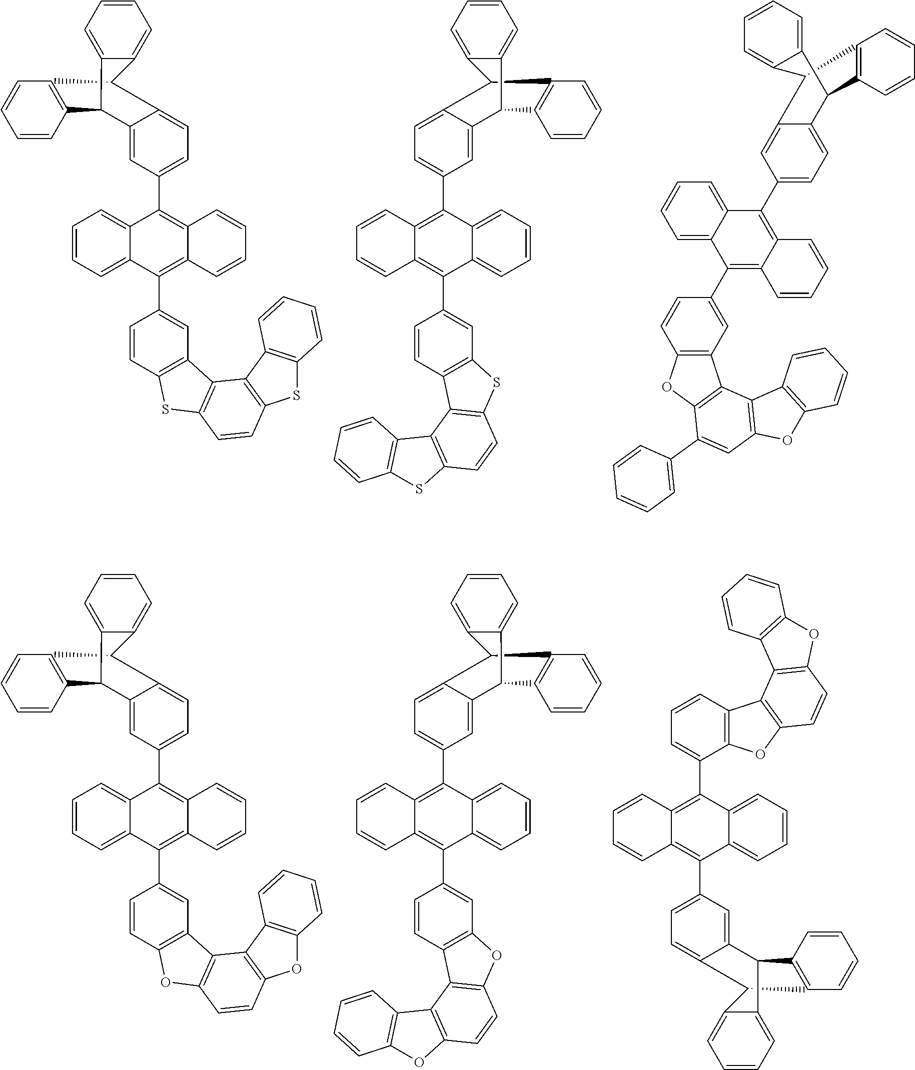

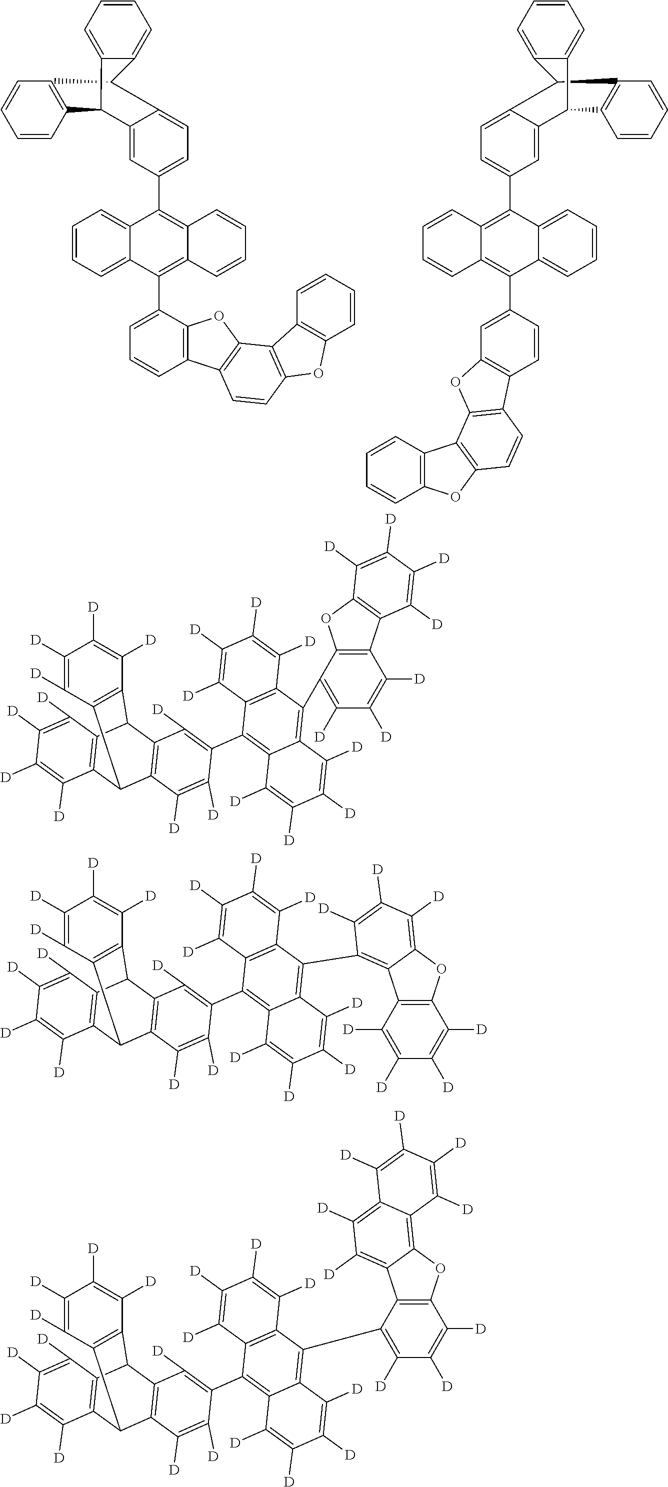

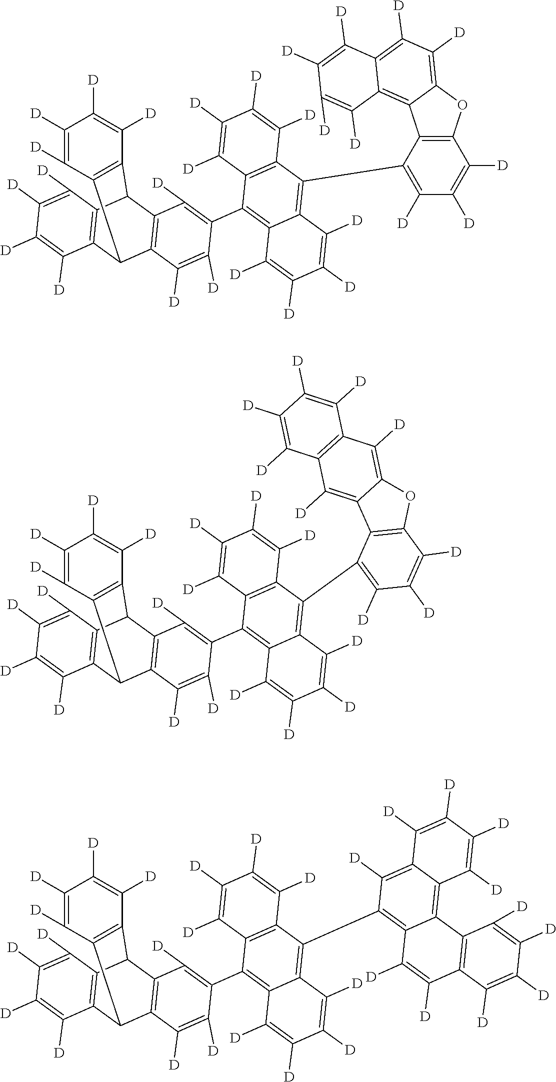

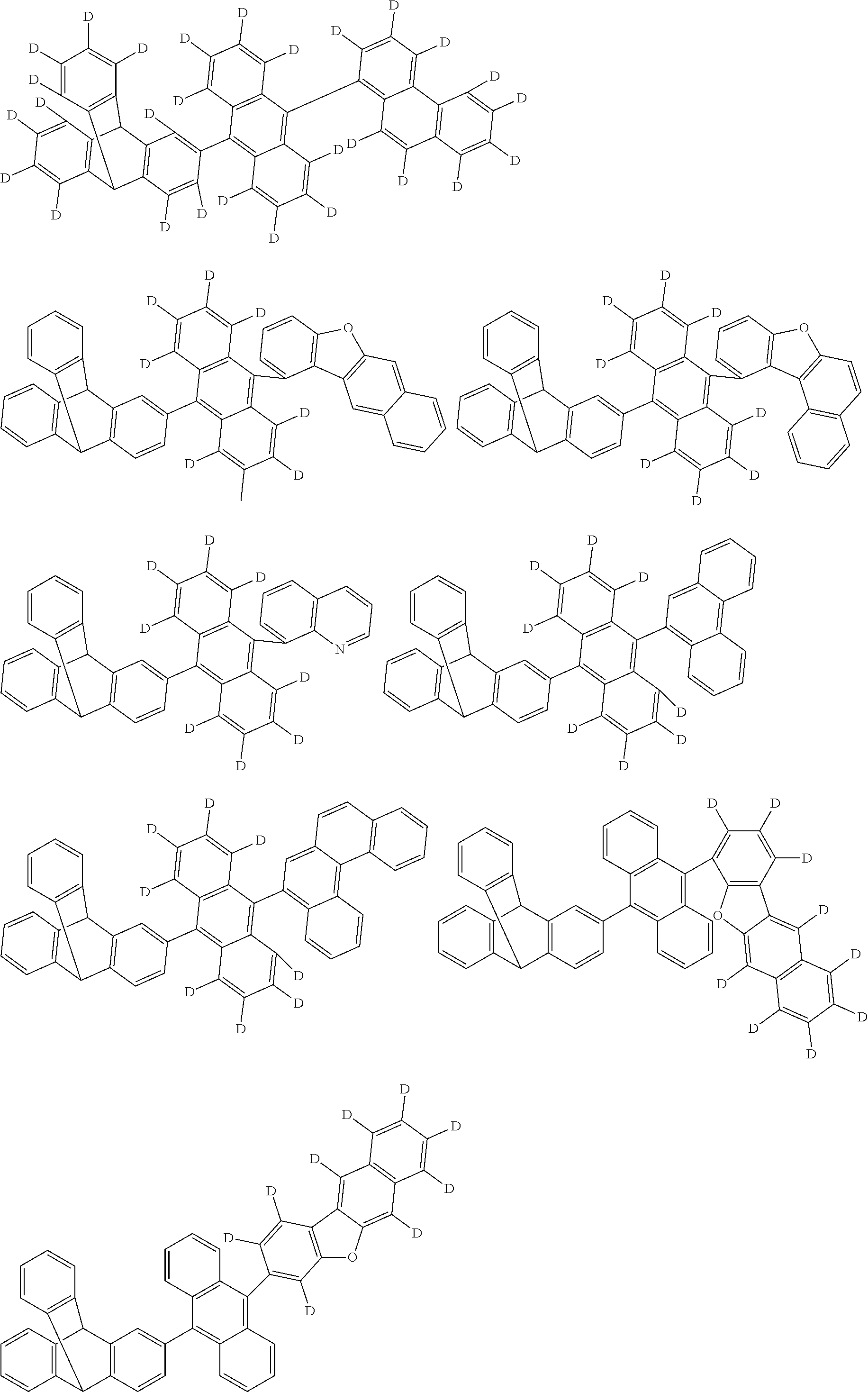

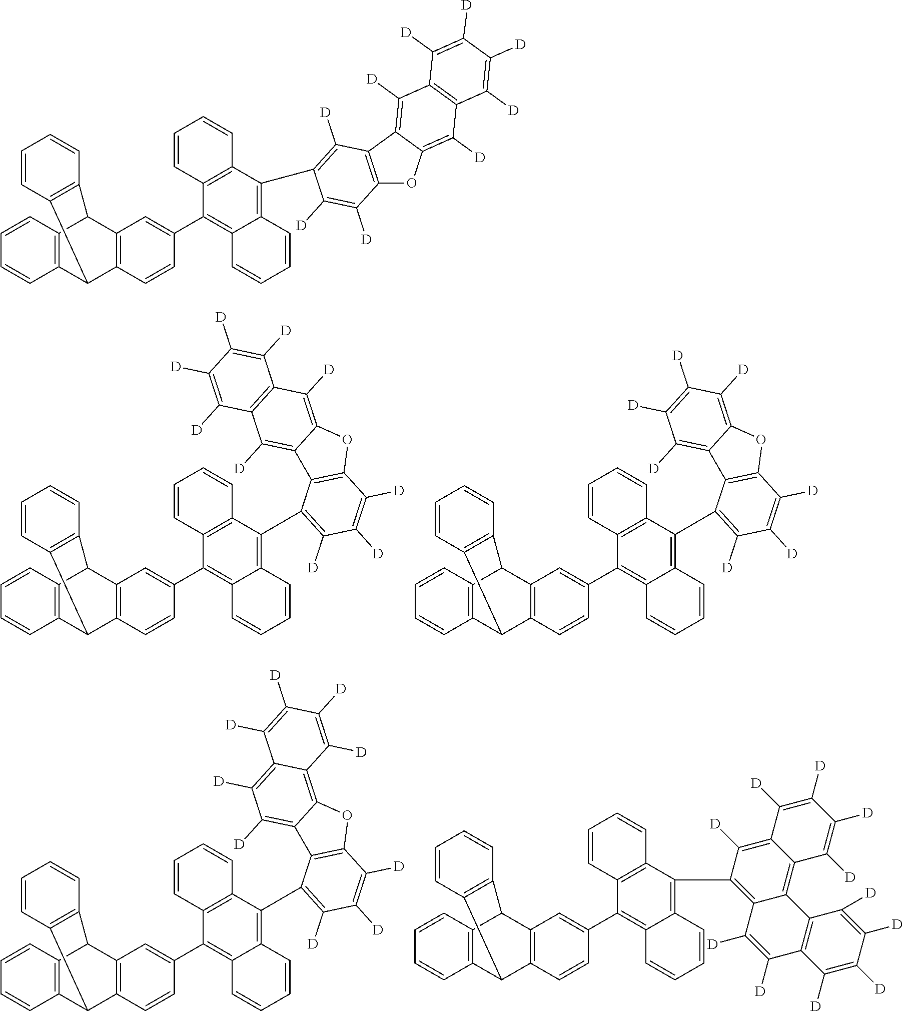

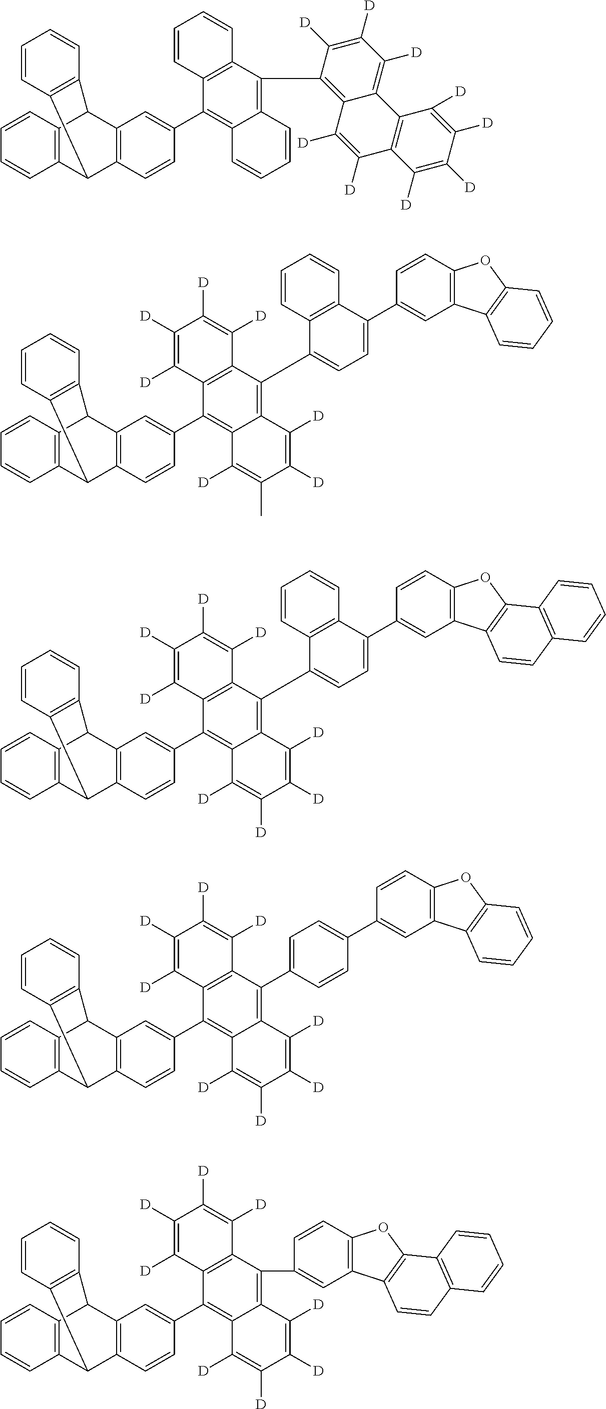

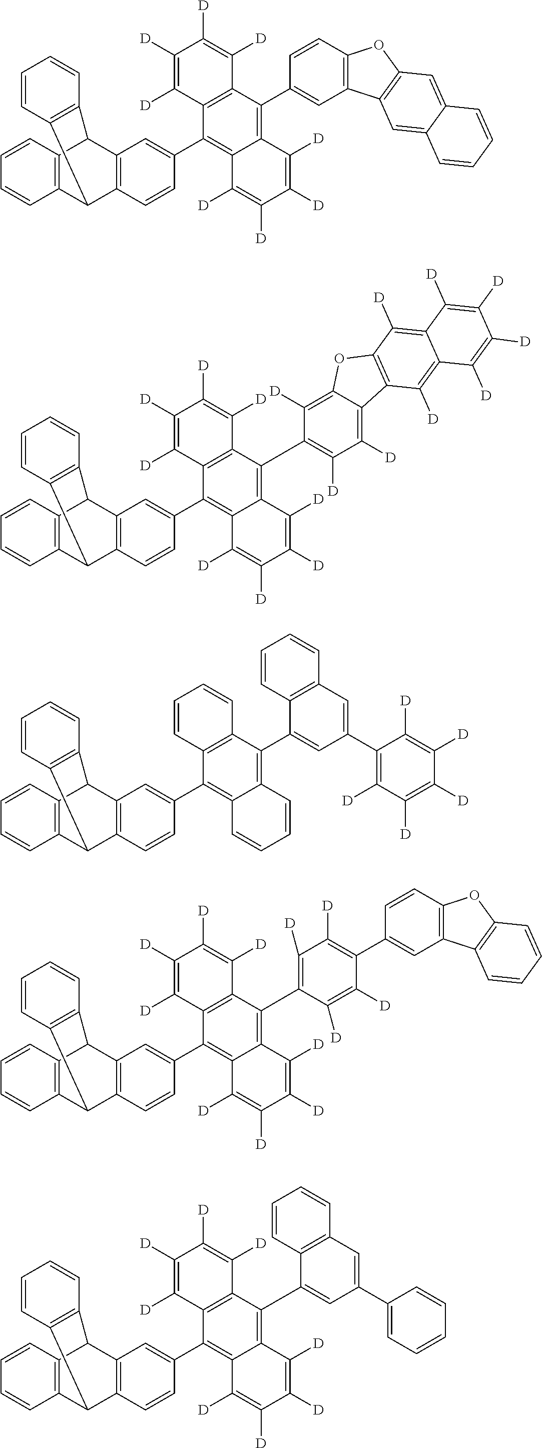

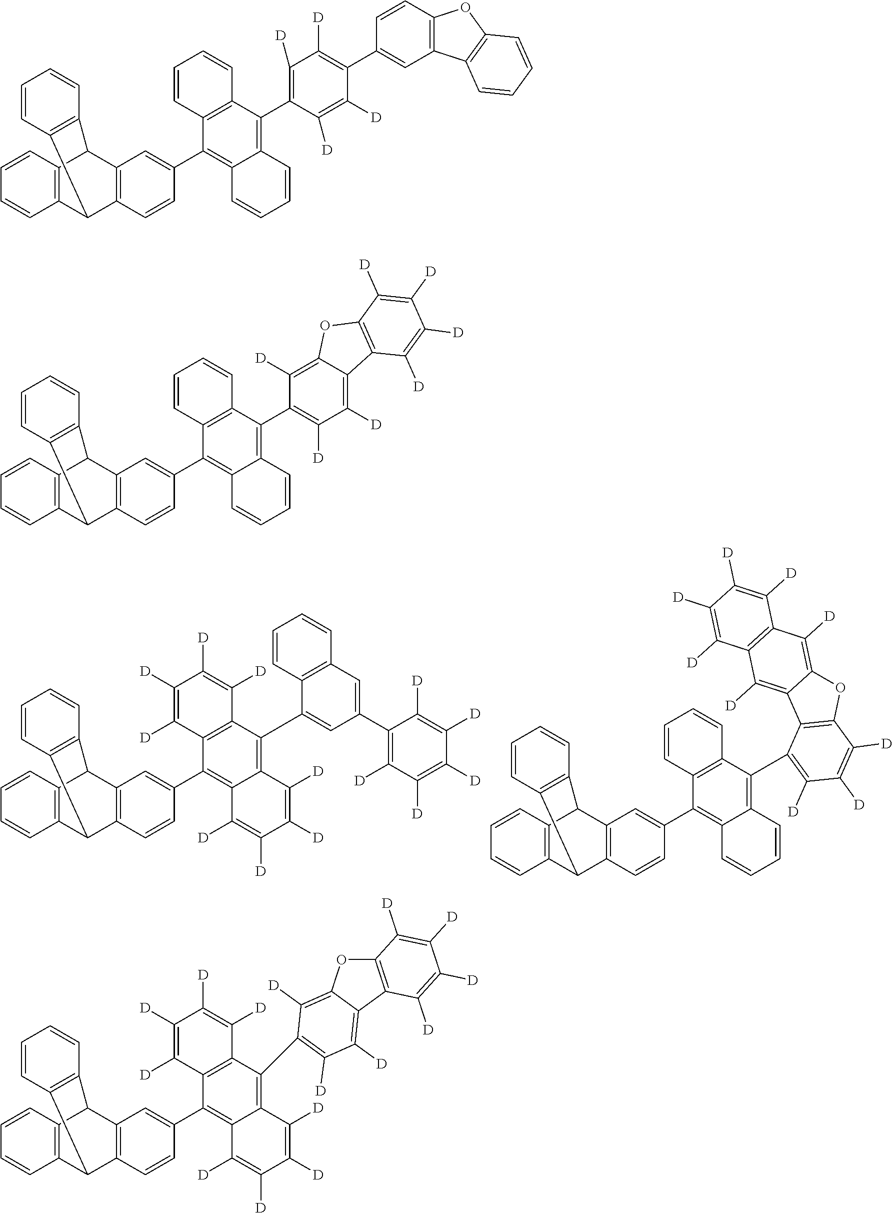

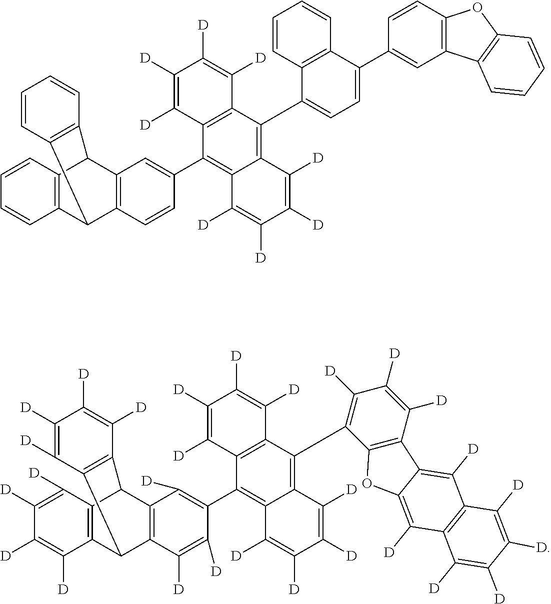

11. An organic electroluminescent device, comprising an anode, an organic light-emitting layer, and a cathode sequentially stacked;
wherein the organic light-emitting layer contains a compound represented by Chemical Formula 1: 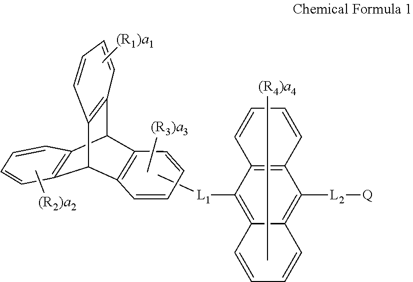
wherein, L₁ and L₂ are each independently selected from a single bond or a linking group, the linking group being selected from divalent arylene or divalent heteroarylene;
Q is a substituted or unsubstituted 2 to 5 ring-fused aryl group, or a substituted or unsubstituted 2 to 5 ring-fused heteroaryl group;
R₁ to R₃ are the same or different from each other and are each independently selected from a group consisting of deuterium, fluorine, chlorine, substituted or unsubstituted alkyl having 1 to 12 carbon atoms, substituted or unsubstituted haloalkyl having 1 to 12 carbon atoms, substituted or unsubstituted alkoxy having 1 to 12 carbon atoms, substituted or unsubstituted cycloalkyl having 3 to 10 carbon atoms, substituted or unsubstituted aryl having 6 to 60 carbon atoms, and substituted or unsubstituted heteroaryl having 3 to 60 carbon atoms;
R₄ is selected from deuterium, halogen, cyano, substituted or unsubstituted alkylamino having 1 to 20 carbon atoms, substituted or unsubstituted alkyl having 1 to 20 carbon atoms, substituted or unsubstituted alkoxy having 1 to 20 carbon atoms, substituted or unsubstituted aryloxy having 6 to 30 ring carbon atoms, substituted or unsubstituted arylthio having 6 to 30 ring carbon atoms, substituted or unsubstituted aryl having 6 to 30 ring carbon atoms, and substituted or unsubstituted heterocyclic group having 5 to 30 ring carbon atoms;
a₁ is an integer from 0 to 4; when a₁ is greater than 1, any two R₁ are the same or different;
a₂ is an integer from 0 to 4; when a₂ is greater than 1, any two R₂ are the same or different;
a₃ is an integer from 0 to 3; when a₃ is greater than 1, any two R₃ are the same or different;
a₄ is an integer from 0 to 8; when a₄ is greater than 1, any two R₄ are the same or different.

12. The organic electroluminescent device according to claim 11, wherein Q is selected from a group consisting of the groups represented by Chemical Formulas 3-1 to 3-13: 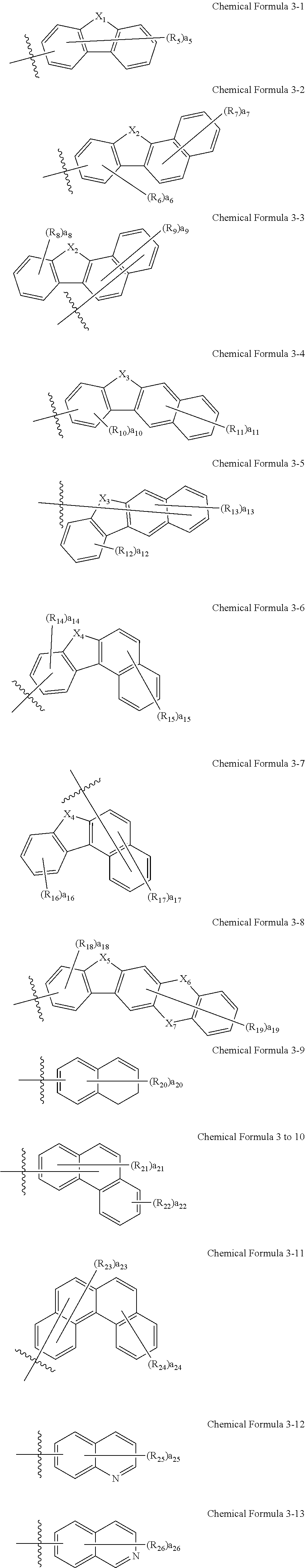
wherein, X to X₅ are each independently selected from, S, C(R)₂, N(R), or Si(R)₂;
X₆ to X₇ are each independently selected from a single bond, O, S, C(R)₂, N(R), or Si(R)₂, with the proviso that X₆ and X₇ are not simultaneously a single bond;
R is selected from the group consisting of hydrogen, deuterium, fluorine, chlorine, substituted or unsubstituted alkyl having 1 to 12 carbon atoms, substituted or unsubstituted haloalkyl having 1 to 12 carbon atoms, substituted or unsubstituted alkoxy having 1 to 12 carbon atoms, substituted or unsubstituted cycloalkyl having 3 to 10 carbon atoms, substituted or unsubstituted aryl having 6 to 60 carbon atoms, and substituted or unsubstituted heteroaryl having 3 to 60 carbon atoms;
R₅ to R₂₆ are the same or different from each other and are each independently selected from the group consisting of deuterium, fluorine, chlorine, substituted or unsubstituted alkyl having 1 to 12 carbon atoms, substituted or unsubstituted haloalkyl having 1 to 12 carbon atoms, substituted or unsubstituted alkoxy having 1 to 12 carbon atoms, substituted or unsubstituted cycloalkyl having 3 to 10 carbon atoms, substituted or unsubstituted aryl having 6 to 60 carbon atoms, and substituted or unsubstituted heteroaryl having 3 to 60 carbon atoms;
a₅ is an integer from 0 to 7; when a₅ is greater than 1, any two R₅ may be the same or different;
a₆ is an integer from 0 to 3; when a₆ is greater than 1, any two R₆ may be the same or different;
a₇ is an integer from 0 to 6; when a₇ is greater than 1, any two R₇ may be the same or different;
a₈ is an integer from 0 to 4; when a₈ is greater than 1, any two R₈ may be the same or different;
a₉ is an integer from 0 to 5; when a₉ is greater than 1, any two R₉ may be the same or different;
a₁₀ is an integer from 0 to 3; when a₁₀ is greater than 1, any two R₁₀ may be the same or different;
a₁₁ is an integer from 0 to 6; when a₁₁ is greater than 1, any two R₁₁ may be the same or different;
a₁₂ is an integer from 0 to 4; when a₁₂ is greater than 1, any two R₁₂ may be the same or different;
a₁₃ is an integer from 0 to 5; when a₁₃ is greater than 1, any two R₁₃ may be the same or different;
a₁₄ is an integer from 0 to 3; when a₁₄ is greater than 1, any two R₁₄ may be the same or different;
a₁₅ is an integer from 0 to 6; when a₁₅ is greater than 1, any two R₁₅ may be the same or different;
a₁₆ is an integer from 0 to 4; when a₁₆ is greater than 1, any two R₁₆ may be the same or different;
a₁₇ is an integer from 0 to 5; when a₁₇ is greater than 1, any two R₁₇ may be the same or different;
a₁₈ is an integer from 0 to 3; when a₁₈ is greater than 1, any two R₁₈ may be the same or different;
a₁₉ is an integer from 0 to 6; when a₁₉ is greater than 1, any two R₁₉ may be the same or different;
a₂₀ is an integer from 0 to 7; when a₂₀ is greater than 1, any two R₂₀ may be the same or different;
a₂₁ is an integer from 0 to 5; when a₂₁ is greater than 1, any two R₂₁ may be the same or different;
a₂₂ is an integer from 0 to 4; when a₂₂ is greater than 1, any two R₂₂ may be the same or different;
a₂₃ is an integer from 0 to 5; when a₂₃ is greater than 1, any two R₂₃ may be the same or different;
a₂₄ is an integer from 0 to 6; when a₂₄ is greater than 1, any two R₂₄ may be the same or different;
a₂₅ is an integer from 0 to 6; when a₂₅ is greater than 1, any two R₂₅ may be the same or different;
a₂₆ is an integer from 0 to 6; when a₂₆ is greater than 1, any two R₂₆ may be the same or different.

13. The compound according to claim 12, wherein Q is selected from the group consisting of a group represented by Chemical Formula 3-1, a group represented by Chemical Formula 3-2, a group represented by Chemical Formula 3-4, a group represented by Chemical Formula 3-6, a group represented by Chemical Formula 3-8a, a group represented by Chemical Formula 3-8b, a group represented by Chemical Formula 3-9, a group represented by Chemical Formula 3-10, a group represented by Chemical Formula 3-11a, a group represented by Chemical Formula 3-12a, and a group represented by Chemical Formula 3-13;

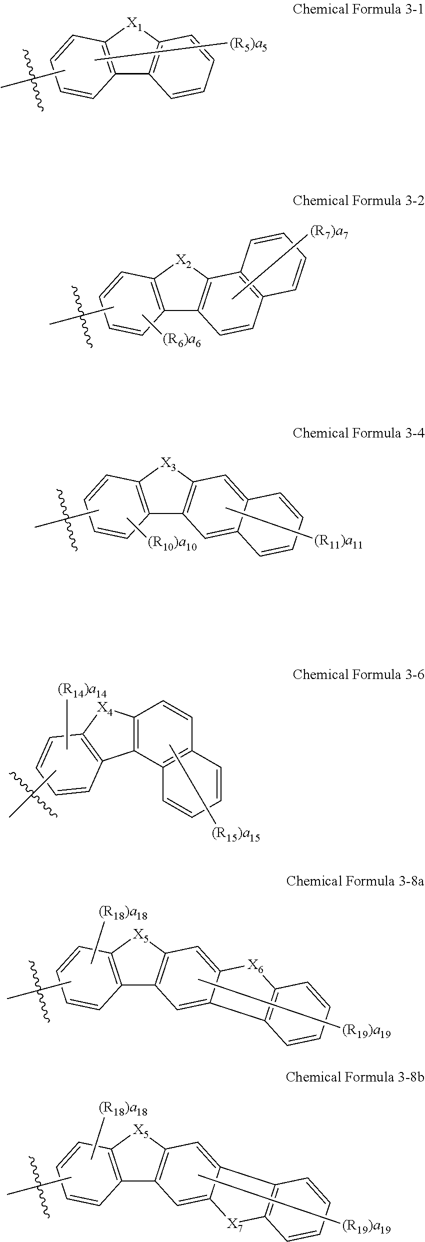

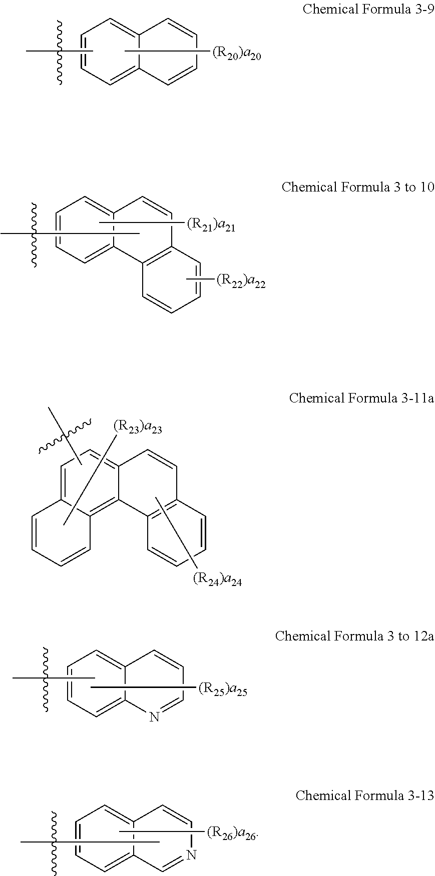

14. The organic electroluminescent device according to claim 12, wherein X₁ to X₅ are each independently selected from O or S; X₆ to X₇ are each independently selected from a single bond, O, or S, with the proviso that X₆ and X₇ are not simultaneously a single bond.

15. The organic electroluminescent device according to claim 13, wherein X₁ to X₅ are each independently selected from O or S; X₆ to X₇ are each independently selected from a single bond, O, or S, with the proviso that X₆ and X₇ are not simultaneously a single bond.

16. The organic electroluminescent device according to claim 12, wherein R₅ to R₂₆ are each independently selected from the group consisting of deuterium, fully deuterated or non-deuterated methyl, and aryl groups, wherein the aryl groups are selected from fully deuterated or non-deuterated phenyl, fully deuterated or non-deuterated biphenyl, fully deuterated or non-deuterated naphthyl, fully deuterated or non-deuterated anthryl, fully deuterated or non-deuterated phenanthryl, and groups formed by connecting any two or three of the above via single bonds.

17. The organic electroluminescent device according to claim 13, wherein R₅ to R₂₆ are each independently selected from the group consisting of deuterium, fully deuterated or non-deuterated methyl, and aryl groups, wherein the aryl groups are selected from fully deuterated or non-deuterated phenyl, fully deuterated or non-deuterated biphenyl, fully deuterated or non-deuterated naphthyl, fully deuterated or non-deuterated anthryl, fully deuterated or non-deuterated phenanthryl, and groups formed by connecting any two or three of the above via single bonds.

18. The organic electroluminescent device according to claim 11, wherein L₁ and L₂ are each independently selected from a single bond, fully deuterated or non-deuterated phenylene, fully deuterated or non-deuterated biphenylene, fully deuterated or non-deuterated naphthylene, fully deuterated or non-deuterated anthrylene, fully deuterated or non-deuterated phenanthrylene, and groups formed by connecting any two or three of the above via single bonds.

19. The organic electroluminescent device according to claim 11, wherein R₁ to R₄ are all deuterium; a₁ is 0 or 4; a₂ is 0 or 4; a₃ is 0 or 3; a₄ is 0 or 8; and
a₁ to a₃ are simultaneously 0, or simultaneously non-zero.

20. A display apparatus, comprising an organic electroluminescent device;
wherein the electroluminescent device comprises an anode, an organic light-emitting layer, and a cathode sequentially stacked;
the organic light-emitting layer contains a compound represented by Chemical Formula 1: 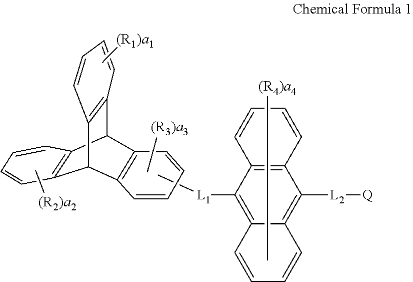
wherein, L₁ and L₂ are each independently selected from a single bond or a linking group, the linking group being selected from divalent arylene or divalent heteroarylene;
Q is a substituted or unsubstituted 2 to 5 ring-fused aryl group, or a substituted or unsubstituted 2 to 5 ring-fused heteroaryl group;
R₁ to R₃ are the same or different from each other and are each independently selected from a group consisting of deuterium, fluorine, chlorine, substituted or unsubstituted alkyl having 1 to 12 carbon atoms, substituted or unsubstituted haloalkyl having 1 to 12 carbon atoms, substituted or unsubstituted alkoxy having 1 to 12 carbon atoms, substituted or unsubstituted cycloalkyl having 3 to 10 carbon atoms, substituted or unsubstituted aryl having 6 to 60 carbon atoms, and substituted or unsubstituted heteroaryl having 3 to 60 carbon atoms;
R₄ is selected from deuterium, halogen, cyano, substituted or unsubstituted alkylamino having 1 to 20 carbon atoms, substituted or unsubstituted alkyl having 1 to 20 carbon atoms, substituted or unsubstituted alkoxy having 1 to 20 carbon atoms, substituted or unsubstituted aryloxy having 6 to 30 ring carbon atoms, substituted or unsubstituted arylthio having 6 to 30 ring carbon atoms, substituted or unsubstituted aryl having 6 to 30 ring carbon atoms, and substituted or unsubstituted heterocyclic group having 5 to 30 ring carbon atoms;
a₁ is an integer from 0 to 4; when a₁ is greater than 1, any two R₁ are the same or different;
a₂ is an integer from 0 to 4; when a₂ is greater than 1, any two R₂ are the same or different;
a₃ is an integer from 0 to 3; when a₃ is greater than 1, any two R₃ are the same or different;
a₄ is an integer from 0 to 8; when a₄ is greater than 1, any two R₄ are the same or different.
